SEQUENCE LISTING (12 sequences)

SEQ ID NO: 1 - DNA, 18 bp, Artificial Sequence
  PCR PRIMER
ccggctacctgcccattc

SEQ ID NO: 2 - DNA, 22 bp, Artificial Sequence
  PCR PRIMERS
ccagatcatcctgatcgacaag

SEQ ID NO: 3 - DNA, 24 bp, Artificial Sequence
  PCR PRIMERS
acatcgcatcgagcgagcacgtac

SEQ ID NO: 4 - DNA, 24 bp, Artificial Sequence
  PCR PRIMERS
cgcgtctcctttgagctgtttgca

SEQ ID NO: 5 - DNA, 15 bp, Artificial Sequence
  PCR PRIMERS
acggcgagcccttgg

SEQ ID NO: 6 - DNA, 21 bp, Artificial Sequence
  PCR PRIMERS
tttctgctgtctttgggacct

SEQ ID NO: 7 - DNA, 17 bp, Artificial Sequence
  PCT PRIMERS
atggacaggcgccctga

SEQ ID NO: 8 - DNA, 20 bp, Artificial Sequence
  PCT PRIMERS
ttgatgggcagcttggtttc

SEQ ID NO: 9 - DNA, 17 bp, Artificial Sequence
  PCR PRIMERS
cacgccatgcgctgcgg

SEQ ID NO: 10 - DNA, 19 bp, Artificial Sequence
  PCR PRIMERS
tgcggaaccggtgagtaca

SEQ ID NO: 11 - DNA, 21 bp, Artificial Sequence
  PCR PRIMERS
gcgggtttatccaagaaagga

SEQ ID NO: 12 - DNA, 21 bp, Artificial Sequence
  PCR PRIMERS
cggaattgccaggacgaccgg

(19) United States
(12) Patent Application Publication
(10) Pub. No.: US 2016/0257697 A1
(43) Pub. Date: Sep. 8, 2016

(54) THIOPHENE-SUBSTITUTED TETRACYCLIC COMPOUNDS AND METHODS OFUSE THEREOF FOR THE TREATMENT OF VIRAL DISEASES

(71) Applicant: MERCK SHARP & DOHME CORP., Rahway, NJ (US)

(72) Inventors: Ling Tong, Warren, NJ (US); Wensheng Yu, Edison, NJ (US); Joseph A. Kozlowski, Princeton, NJ (US); Lei Chen, Roselle Park, NJ (US); Oleg Selyutin, West Windsor, NJ (US); Seong Heon Kim, Rahway, NJ (US); Michael Dwyer, Scotch Plains, NJ (US); Bin Hu, Shanghai (CN); Bin Zhong, Shanghai (CN); Dahai Wang, Shanghai (CN); Jinglai Hao, Shanghai (CN); Changmao Shen, Shanghai (CN); Zhixin Lei, Shanghai (CN); Weijun Wang, Shanghai (CN)

(73) Assignee: MERCK SHARP & DOHME CORP., Rahway, NJ (US)

(21) Appl. No.: 14/761,259

(22) PCT Filed: Dec. 31, 2013
(86) PCT No.: PCT/CN2013/001677

§ 371 Date: Jul. 15, 2015

(30) Foreign Application Priority Data
Jan. 16, 2013 (CN) PCT/CN2013/000039

(51) Int. Cl. C07D 498/04; C07D 519/00; A61K 45/06; A61K 31/7056; A61K 31/7072; A61K 38/00; A61K 31/5365; A61K 38/21

(52) U.S. Cl. CPC C07D 498/04; A61K 31/5365; C07D 519/00; A61K 38/212; A61K 31/7056; A61K 31/7072; A61K 38/005; A61K 45/06

(57) ABSTRACT

Thiophene-substituted tetracyclic compounds of formula (I) and pharmaceutically acceptable salts thereof, wherein A, A′, R², R³, R⁴ and R⁵ are as defined herein. The compositions comprising at least one thiophene-substituted tetracyclic compound, and methods of using the thiophene-substituted tetracyclic compounds for treating or preventing HCV infection in a patient are also provided.

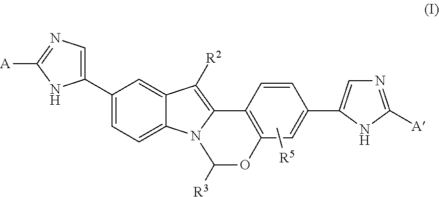

### FIELD OF THE INVENTION

The present invention relates to novel Thiophene-Substituted Tetracyclic Compounds, compositions comprising at least one Thiophene-Substituted Tetracyclic Compound, and methods of using the Thiophene-Substituted Tetracyclic Compounds for treating or preventing HCV infection in a patient.

### BACKGROUND OF THE INVENTION

Hepatitis C virus (HCV) is a major human pathogen. A substantial fraction of these HCV-infected individuals develop serious progressive liver disease, including cirrhosis and hepatocellular carcinoma, which are often fatal.

Recent attention has been focused toward the identification of inhibitors of HCV NS5A. HCV NS5A is a 447 amino acid phosphoprotein which lacks a defined enzymatic function. It runs as 56kd and 58kd bands on gels depending on phosphorylation state (Tanji, et al. J. Virol. 69:3980-3986 (1995)). HCV NS5A resides in replication complex and may be responsible for the switch from replication of RNA to production of infectious virus (Huang, Y, et al., Virology 364:1-9 (2007)).

Multicyclic HCV NS5A inhibitors have been reported. See U.S. Patent Publication Nos. US20080311075, US20080044379, US20080050336, US20080044380, US20090202483 and US2009020478. HCV NS5A inhibitors having fused tricyclic moieties are disclosed in International Patent Publication Nos. WO 10/065681, WO 10/065668, and WO 10/065674.

Other HCV NS5A inhibitors and their use for reducing viral load in HCV infected humans have been described in U.S. Patent Publication No. US20060276511.

### SUMMARY OF THE INVENTION

In one aspect, the present invention provides Compounds of Formula

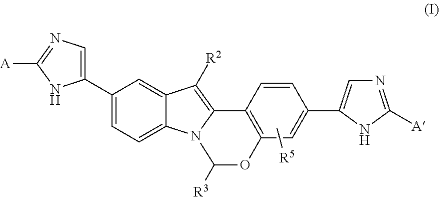

or a pharmaceutically acceptable salt thereof, wherein:

A is:

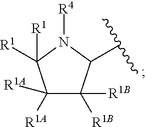

A′ is:

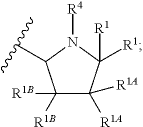

each occurrence of R¹ is independently selected from H, C₁-C₆ alkyl, C₃-C₇ cycloalkyl, C₁-C₆ haloalkyl and halo, or two R¹ groups that are attached to the same carbon atom, and the common carbon atom to which they are attached, can combine to form a spirocyclic C₃-C₇ cycloalkyl group;

each occurrence of R^{1A} is independently selected from H, C₁-C₆ alkyl, C₃-C₇ cycloalkyl, C₁-C₆ haloalkyl and halo, or one R^{1A} group and an R¹ group that are attached to same ring, together with the ring carbon atoms to which they are attached, can combine to form a fused C₃-C₇ cycloalkyl group, or two R^{1A} groups that are attached to the same carbon atom, and the common carbon atom to which they are attached, can combine to form a spirocyclic C₃-C₇ cycloalkyl group;

each occurrence of R^{1B} is independently H, C₁-C₆ alkyl, C₃-C₇ cycloalkyl, C₁-C₆ haloalkyl or halo, or an R^{1B} group and an R^{1A} group that are attached to the same ring, together with the carbon atoms to which they are attached, can combine to form a fused C₃-C₇ cycloalkyl group, or an R^{1B} group and an R¹ group that are attached to the same ring, can combine to form a bridging group having the formula —CH₂— or —CH₂CH₂—, or or two R^{1B} groups that are attached to the same carbon atom, and the common carbon atom to which they are attached, can combine to form a spirocyclic C₃-C₇ cycloalkyl group

R² is H, C₁-C₆ alkyl, C₃-C₇ cycloalkyl, phenyl or halo;

R³ is thiophenyl, wherein said thiophenyl group can be optionally substituted on one or more ring carbon atoms with R⁶;

each occurrence of R⁴ is independently selected from —C(O)O—(C₁-C₆ alkyl), —C(O)—C(R⁷)₂NHC(O)O—R⁸, —C(O)—CH(R⁷)(R⁸) and —C(O)—CH(R⁷)N(R⁹)₂;

R⁵ represents up to 2 substituents, each independently selected from H, halo, —CN, C₁-C₆ alkyl, C₁-C₆ haloalkyl, —(C₁-C₆ alkylene)ₘ-C₃-C₇ cycloalkyl, 4 to 6-membered monocyclic heterocycloalkyl, 5 or 6-membered monocyclic heteroaryl, C₆-C₁₀ aryl, benzyl and —O—(C₁-C₆ alkyl), wherein said C₃-C₇ cycloalkyl group, said 4 to 6-membered monocyclic heterocycloalkyl group, said 5 or 6-membered monocyclic heteroaryl group, said C₆-C₁₀ aryl group, or the phenyl moiety of said benzyl group can be optionally substituted with up to 3 groups, which can be the same or different, and are selected from halo, —CN, C₁-C₆ alkyl, C₁-C₆ haloalkyl, —O—C₁-C₆ alkyl, —(C₁-C₆ alkylene)-O—C₁-C₆ alkyl and —O—(C₁-C₆ haloalkyl);

R⁶ represents up to 2 substituents, each independently selected from halo, —CN, C₁-C₆ alkyl, —C(O)OH, C₁-C₆ haloalkyl, —O—(C₁-C₆ haloalkyl), C₂-C₆ alkynyl, C₁-C₆ hydroxyalkyl, —O—C₁-C₆ alkyl, —(C₁-C₆ alkylene)-O—(C₁-C₆ alkyl), —N(R⁶)₂, —C(O)N(R⁶)₂, optionally substituted C₆-C₁₀ aryl, —(C₁-C₆ alkylene)ₘ-(C₃-C₇ cycloalkyl), —O—(C₆-C₁₀ aryl), —(C₂-C₆ alkynyl)-(C₃-C₇ cycloalkyl), 4 to 7-membered monocyclic heterocycloalkyl, 5 or 6-membered monocyclic heteroaryl, —O-(5 or 6-membered monocyclic heteroaryl), 8 to 10-membered bicyclic heteroaryl and —O-(8 to 10-membered bicyclic heteroaryl), wherein said C₆-C₁₀ aryl group, said C₃-C₇ cycloalkyl group, said 4 to 7-membered monocyclic heterocycloalkyl group, said 5 or 6-membered monocyclic heteroaryl group and said 8 to 10-membered bicyclic heteroaryl group, can be optionally substituted with up to 3 groups, each independently selected from halo, hydroxy, C₁-C₆ alkyl, C₁-C₆ haloalkyl and —O—C₁-C₆ alkyl, and wherein said C₆-C₁₀ aryl group, said 5 or 6-membered monocyclic heteroaryl group and said 9- or 10-membered bicyclic heteroaryl group, can be optionally fused with a 3 to 6 membered cycloalkyl group; and wherein said thiophenyl group can be optionally fused to a benzene ring, a 5 or 6-membered monocyclic heterocycloalkyl group, a 5 or 6-membered monocyclic heteroaryl group or a C₅-C₆ cycloalkyl group, wherein said 5 or 6-membered monocyclic heterocycloalkyl group, said 5 or 6-membered monocyclic heteroaryl group and said C₅-C₆ cycloalkyl group can form a spirocycle with a C₃-C₇ cycloalkyl group or a 4 to 7-membered monocyclic heterocycloalkyl group each occurrence of R⁷ is independently selected from H, C₁-C₆ alkyl, C₁-C₆ haloalkyl, —(C₁-C₆ alkylene)-O—C₁-C₆ alkyl, phenyl, 4 to 8-membered monocyclic heterocycloalkyl, 6 to 10-membered bicyclic heterocycloalkyl and —(C₁-C₆ alkylene)ₘ-C₃-C₇ cycloalkyl, wherein said 4 to 8-membered monocyclic heterocycloalkyl group, said 6 to 10-membered bicyclic heterocycloalkyl group and said C₃-C₇ cycloalkyl group can be optionally substituted with up to 5 groups, each independently selected from halo, —CN, C₁-C₆ alkyl, C₃-C₇ cycloalkyl, C₁-C₆ haloalkyl, —O—C₁-C₆ alkyl, —N(R⁶)₂ and —O—(C₁-C₆ haloalkyl), and wherein said C₃-C₇ cycloalkyl group can be optionally fused to a 4 to 6-membered monocyclic heterocycloalkyl group, and wherein said 4 to 8-membered monocyclic heterocycloalkyl group and said C₃-C₇ cycloalkyl group can be substituted on a ring carbon atom with a spirocyclic C₃-C₆ cycloalkyl group; and wherein said C₃-C₇ cycloalkyl group can be substituted on a ring carbon atom with a spirocyclic 3 to 6-membered monocyclic heterocycloalkyl group, and wherein two R⁷ groups, that are attached to a common carbon atom, together with the common carbon atom to which they are attached, join to form a C₃-C₇ cycloalkyl group;

each occurrence of R⁸ is independently selected from C₁-C₆ alkyl, C₃-C₇ cycloalkyl and C₆-C₁₀ aryl;

each occurrence of R⁹ is independently selected from H, C₁-C₆ alkyl, C₃-C₇ cycloalkyl and C₆-C₁₀ aryl; and

each occurrence of m is independently 0 or 1.

The Compounds of Formula (I) (also referred to herein as the “Thiophene-Substituted Tetracyclic Compounds”) and pharmaceutically acceptable salts thereof can be useful, for example, for inhibiting HCV viral replication or replicon activity, and for treating or preventing HCV infection in a patient. Without being bound by any specific theory, it is believed that the Thiophene-Substituted Tetracyclic Compounds inhibit HCV viral replication by inhibiting HCV NS5A.

Accordingly, the present invention provides methods for treating or preventing HCV infection in a patient, comprising administering to the patient an effective amount of at least one Thiophene-Substituted Tetracyclic Compound.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### DETAILED DESCRIPTION OF THE INVENTION

The present invention relates to novel Thiophene-Substituted Tetracyclic Compounds, compositions comprising at least one Thiophene-Substituted Tetracyclic Compound, and methods of using the Thiophene-Substituted Tetracyclic Compounds for treating or preventing HCV infection in a patient.

### DEFINITIONS AND ABBREVIATIONS

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc. . . . .

As used herein, and throughout this disclosure, the following terms, unless otherwise indicated, shall be understood to have the following meanings:

A “patient” is a human or non-human mammal. In one embodiment, a patient is a human. In another embodiment, a patient is a chimpanzee.

The term “effective amount” as used herein, refers to an amount of Thiophene-Substituted Tetracyclic Compound and/or an additional therapeutic agent, or a composition thereof that is effective in producing the desired therapeutic, ameliorative, inhibitory or preventative effect when administered to a patient suffering from a viral infection or virus-related disorder. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an HCV viral infection or HCV-virus related disorder, refers to reducing the likelihood of HCV infection.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)— cycloalkyl, —C(O)OH and —C(O)O-alkyl. The term “C₂-C₆ alkenyl” refers to an alkenyl group having from 2 to 6 carbon atoms. Unless otherwise indicated, an alkenyl group is unsubstituted.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkynyl” refers to an alkynyl group having from 2 to 6 carbon atoms. Unless otherwise indicated, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. In one embodiment, an aryl group is phenyl. Unless otherwise indicated, an aryl group is unsubstituted.

The term “arylene,” as used herein, refers to a bivalent group derived from an aryl group, as defined above, by removal of a hydrogen atom from a ring carbon of an aryl group. An arylene group can be derived from a monocyclic or multicyclic ring system comprising from about 6 to about 14 carbon atoms. In one embodiment, an arylene group contains from about 6 to about 10 carbon atoms. In another embodiment, an arylene group is a naphthylene group. In another embodiment, an arylene group is a phenylene group. An arylene group can be optionally substituted with one or more “ring system substituents” which may be the same or different, and are as defined herein below. An arylene group is divalent and either available bond on an arylene group can connect to either group flanking the arylene group. For example, the group “A-arylene-B,” wherein the arylene group is:

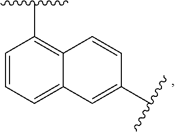 [SMILES: *c1ccc2c(*)cccc2c1]

is understood to represent both:

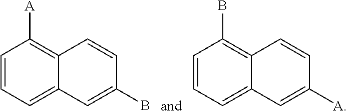

In one embodiment, an arylene group can be optionally fused to a cycloalkyl or cycloalkanoyl group. Non-limiting examples of arylene groups include phenylene and naphthalene. In one embodiment, an arylene group is unsubstituted. In another embodiment, an arylene group is:

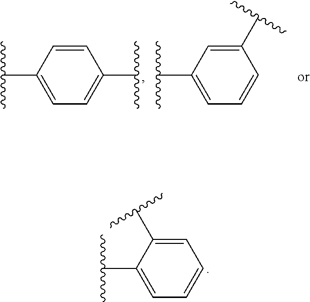

Unless otherwise indicated, an arylene group is unsubstituted.

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 3 to about 7 ring atoms. In another embodiment, a cycloalkyl contains from about 5 to about 6 ring atoms. The term “cycloalkyl” also encompasses a cycloalkyl group, as defined above, which is fused to an aryl (e.g., benzene) or heteroaryl ring. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted. The term “3 to 6-membered cycloalkyl” refers to a cycloalkyl group having from 3 to 6 ring carbon atoms. Unless otherwise indicated, a cycloalkyl group is unsubstituted. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

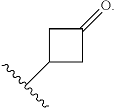 [SMILES: *C1CC(=O)C1]

The term “cycloalkenyl,” as used herein, refers to a non-aromatic mono- or multicyclic ring system comprising from about 4 to about 10 ring carbon atoms and containing at least one endocyclic double bond. In one embodiment, a cycloalkenyl contains from about 4 to about 7 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. A ring carbon atom of a cycloalkyl group may be functionalized as a carbonyl group. In one embodiment, a cycloalkenyl group is cyclopentenyl. In another embodiment, a cycloalkenyl group is cyclohexenyl. The term “4 to 6-membered cycloalkenyl” refers to a cycloalkenyl group having from 4 to 6 ring carbon atoms. Unless otherwise indicated, a cycloalkenyl group is unsubstituted.

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic and had 9 or 10 ring atoms. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered heteroaryl. In another embodiment, a heteroaryl group comprises a 5- to 6-membered heteroaryl group fused to a benzene ring. Unless otherwise indicated, a heteroaryl group is unsubstituted.

The term “heteroarylene,” as used herein, refers to a bivalent group derived from an heteroaryl group, as defined above, by removal of a hydrogen atom from a ring carbon or ring heteroatom of a heteroaryl group. A heteroarylene group can be derived from a monocyclic or multicyclic ring system comprising about 5 to about 14 ring atoms, wherein from 1 to 4 of the ring atoms are each independently 0, N or S and the remaining ring atoms are carbon atoms. A heteroarylene group can be optionally substituted by one or more “ring system substituents” which may be the same or different, and are as defined herein below. A heteroarylene group is joined via a ring carbon atom or by a nitrogen atom with an open valence, and any nitrogen atom of a heteroarylene can be optionally oxidized to the corresponding N-oxide. The term “heteroarylene” also encompasses a heteroarylene group, as defined above, which is fused to a benzene ring. Non-limiting examples of heteroarylenes include pyridylene, pyrazinylene, furanylene, thienylene, pyrimidinylene, pyridonylene (including those derived from N-substituted pyridonyls), isoxazolylene, isothiazolylene, oxazolylene, oxadiazolylene, thiazolylene, pyrazolylene, thiophenylene, furazanylene, pyrrolylene, triazolylene, 1,2,4-thiadiazolylene, pyrazinylene, pyridazinylene, quinoxalinylene, phthalazinylene, oxindolylene, imidazo[1,2-a]pyridinylene, imidazo[2,1-b]thiazolylene, benzofurazanylene, indolylene, azaindolylene, benzimidazolylene, benzothienylene, quinolinylene, imidazolylene, benzimidazolylene, thienopyridylene, quinazolinylene, thienopyrimidylene, pyrrolopyridylene, imidazopyridylene, isoquinolinylene, benzoazaindolylene, 1,2,4-triazinylene, benzothiazolylene and the like, and all isomeric forms thereof. The term “heteroarylene” also refers to partially saturated heteroarylene moieties such as, for example, tetrahydroisoquinolylene, tetrahydroquinolylene, and the like. A heteroarylene group is divalent and either available bond on a heteroarylene ring can connect to either group flanking the heteroarylene group. For example, the group “A-heteroarylene-B,” wherein the heteroarylene group is:

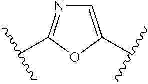 [SMILES: *c1cnc(*)o1]

is understood to represent both:

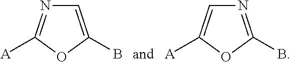

In one embodiment, a heteroarylene group is a monocyclic heteroarylene group or a bicyclic heteroarylene group. In another embodiment, a heteroarylene group is a monocyclic heteroarylene group. In another embodiment, a heteroarylene group is a bicyclic heteroarylene group. In still another embodiment, a heteroarylene group has from about 5 to about 10 ring atoms. In another embodiment, a heteroarylene group is monocyclic and has 5 or 6 ring atoms. In another embodiment, a heteroarylene group is bicyclic and has 9 or 10 ring atoms. In another embodiment, a heteroarylene group is a 5-membered monocyclic heteroarylene. In another embodiment, a heteroarylene group is a 6-membered monocyclic heteroarylene. In another embodiment, a bicyclic heteroarylene group comprises a 5 or 6-membered monocyclic heteroarylene group fused to a benzene ring. Unless otherwise indicated, a heteroarylene group is unsubstituted.

The term “heterocycloalkyl,” as used herein, refers to a non-aromatic saturated monocyclic or multicyclic ring system comprising 3 to about 11 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic and has from about 3 to about 7 ring atoms. In another embodiment, a heterocycloalkyl group is monocyclic has from about 4 to about 7 ring atoms. In another embodiment, a heterocycloalkyl group is bicyclic and has from about 7 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, which is fused to an aryl (e.g., benzene) or heteroaryl ring. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone, silacyclopentane, silapyrrolidine and the like, and all isomers thereof. Non-limiting illustrative examples of a silyl-containing heterocycloalkyl group include:

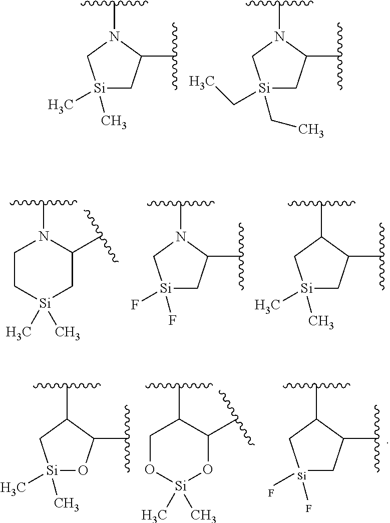

A ring carbon atom of a heterocycloalkyl group may be functionalized as a carbonyl group. An illustrative example of such a heterocycloalkyl group is:

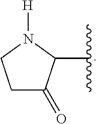 [SMILES: *C1NCCC1=O]

In one embodiment, a heterocycloalkyl group is a 5-membered monocyclic heterocycloalkyl. In another embodiment, a heterocycloalkyl group is a 6-membered monocyclic heterocycloalkyl. The term “3 to 6-membered monocyclic cycloalkyl” refers to a monocyclic heterocycloalkyl group having from 3 to 6 ring atoms. The term “4 to 6-membered monocyclic cycloalkyl” refers to a monocyclic heterocycloalkyl group having from 4 to 6 ring atoms. The term “7 to 11-membered bicyclic heterocycloalkyl” refers to a bicyclic heterocycloalkyl group having from 7 to 11 ring atoms. Unless otherwise indicated, an heterocycloalkyl group is unsubstituted.

The term “heterocycloalkenyl,” as used herein, refers to a heterocycloalkyl group, as defined above, wherein the heterocycloalkyl group contains from 4 to 10 ring atoms, and at least one endocyclic carbon-carbon or carbon-nitrogen double bond. A heterocycloalkenyl group can be joined via a ring carbon or ring nitrogen atom. In one embodiment, a heterocycloalkenyl group has from 4 to 6 ring atoms. In another embodiment, a heterocycloalkenyl group is monocyclic and has 5 or 6 ring atoms. In another embodiment, a heterocycloalkenyl group is bicyclic. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like and the like. A ring carbon atom of a heterocycloalkenyl group may be functionalized as a carbonyl group. In one embodiment, a heterocycloalkenyl group is a 5-membered heterocycloalkenyl. In another embodiment, a heterocycloalkenyl group is a 6-membered heterocycloalkenyl. The term “4 to 6-membered heterocycloalkenyl” refers to a heterocycloalkenyl group having from 4 to 6 ring atoms. Unless otherwise indicated, a heterocycloalkenyl group is unsubstituted.

Examples of “ring system substituents” include, but are not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O— alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, —Si(alkyl)₂, —Si(aryl)₂, —Si(heteroaryl)₂, —Si(alkyl)(aryl), —Si(alkyl)(cycloalkyl), —Si(alkyl)(heteroaryl), cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

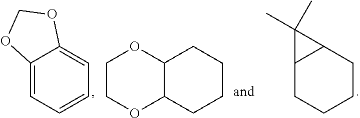

The term “silylalkyl,” as used herein, refers to an alkyl group as defined above, wherein one or more of the alkyl group's hydrogen atoms has been replaced with a —Si(R^{x})₃ group, wherein each occurrence of R^{x} is independently C₁-C₆ alkyl, phenyl or a 3 to 6-membered cycloalkyl group. In one embodiment, a silylalkyl group has from 1 to 6 carbon atoms. In another embodiment, a silyl alkyl group contains a —Si(CH₃)₃ moiety. Non-limiting examples of silylalkyl groups include —CH₂—Si(CH₃)₃ and —CH₂CH₂—Si(CH₃)₃.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any substituent or variable (e.g., alkyl, R⁶, R^{a}, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Thiophene-Substituted Tetracyclic Compound or a pharmaceutically acceptable salt or solvate of the compound. The transformation may occur by various mechanisms (e.g., by metabolic or chemical processes), such as, for example, through hydrolysis in blood.

For example, if a Thiophene-Substituted Tetracyclic Compound or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 6 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Thiophene-Substituted Tetracyclic Compound contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

If a Thiophene-Substituted Tetracyclic Compound incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N- or di-N,N—(C₁-C₆)alkylaminoalkyl; —C(Y⁴)Y⁵ wherein Y⁴ is H or methyl and Y⁵ is mono-N- or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours, 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Thiophene-Substituted Tetracyclic Compounds can form salts which are also within the scope of this invention. Reference to a Thiophene-Substituted Tetracyclic Compound herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Thiophene-Substituted Tetracyclic Compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Thiophene-Substituted Tetracyclic Compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Thiophene-Substituted Tetracyclic Compounds may be atropisomers (e.g., substituted biaryls) and are considered as part of this invention. Enantiomers can also be directly separated using chiral chromatographic techniques.

It is also possible that the Thiophene-Substituted Tetracyclic Compounds may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Thiophene-Substituted Tetracyclic Compound incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the Thiophene-Substituted Tetracyclic Compounds, and of the salts, solvates, hydrates, esters and prodrugs of the Thiophene-Substituted Tetracyclic Compounds, are intended to be included in the present invention.

The following abbreviations are used below and have the following meanings: Ac is acyl; AcCl is acetyl chloride; AcOH or HOAc is acetic acid; Amphos is (4-(N,N)-dimethylaminophenyl)-di-tertbutylphosphine; Aq is aqueous; BF₃.OEt₂ is boron trifluoride etherate; BOC or Boc is tert-butyloxycarbonyl; Boc₂O is Boc anhydride; Boc-Pro-OH is Boc protected proline; L-Boc-Val-OH is Boc protected L-valine; BOP is Benzotriazole-1-yl-oxy-tris-(dimethylamino)-phosphonium hexafluorophosphate; n-BuLi is n-butyllithium; CBZ or Cbz is carbobenzoxy; DCM is dichloromethane; DDQ is 2,3-dichloro-5,6-dicyano-1,4-benzoquinone; Dess-Martin reagent is 1,1-Triacetoxy-1,1-dihydro-1,2-benziodoxol-3(1H)-one; DIPEA is diisopropylethylamine; DME is dimethoxyethane; DMF is N,N-dimethylformamide; dppf is diphenylphosphinoferrocene; DMSO is dimethylsulfoxide; EtMgBr is ethylmagnesium bromide; EtOAc is ethyl acetate; Et₂O is diethyl ether; Et₃N or NEt₃ is triethylamine; HATU is O-(7-azabenzotriazol-1-yl)-N,N,N′,N′-tetramethyluronium hexafluorophosphate; HPLC is high performance liquid chromatography; HRMS is high resolution mass spectrometry; KOAc is potassium acetate; LCMS is liquid chromatography/mass spectrometry; LiHMDS is lithium hexamethyldisilazide; LRMS is low resolution mass spectrometry; MeI is iodomethane; MeOH is methanol; NBS is N-bromosuccinimide; NH₄OAc is ammonium acetate; NMM is N-methylmorpholine; Pd/C is palladium on carbon; Pd(PPh₃)₄ is tetrakis (triphenylphosphine)palladium(0); PdCl₂(dppf)₂ is [1,1′-Bis(diphenylphosphino) ferrocene]dichloro palladium(II); PdCl₂(dppf)₂.CH₂Cl₂ is [1,1′-Bis(diphenylphosphino)ferrocene] dichloro palladium(II) complex with dichloromethane; pinacol₂B₂ is bis(pinacolato)diboron; PPTS is pyridinium p-toluene sulfonate; RPLC is reverse-phase liquid chromatography; Select-F is 1-Chloromethyl-4-Fluoro-1, 4-Diazoniabicyclo[2.2.2]Octane Bis-(Tetrafluoroborate); SEM-Cl is 2-(trimethylsilyl)ethoxymethyl chloride; TBAF is tetrabutylammonium fluoride; TBDMSCk is tert-butyldimethylsilyl chloride; TFA is trifluoroacetic acid; Tf₂O is triflic anhydride; THF is tetrahydrofuran; TLC is thin-layer chromatography; and TosCl is p-toluenesulfonyl chloride.

### The Compounds of Formula (I)

The present invention provides Thiophene-Substituted Tetracyclic Compounds of Formula (I):

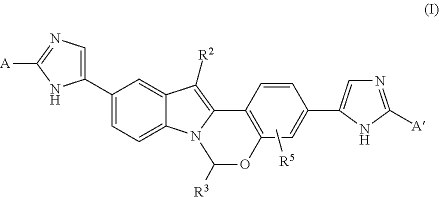

and pharmaceutically acceptable salts thereof, wherein A, A′, R², R³, R⁴ and R⁵ are defined above for the Compounds of Formula (I).

In one embodiment, R² is H

In another embodiment, R² is halo.

In another embodiment, R² is C₁-C₆ alkyl.

In one embodiment, R³ is:

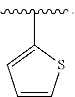 [SMILES: *c1cccs1]

In another embodiment, R³ is:

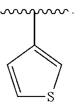 [SMILES: *c1ccsc1]

In one embodiment, R⁵ is H.

In another embodiment, R⁵ is F.

In one embodiment, A and A′ are each a 5-membered heterocycloalkyl group.

In another embodiment, A and A′ are each a 6-membered heterocycloalkyl group.

In another embodiment, A and A′ are each independently selected from:

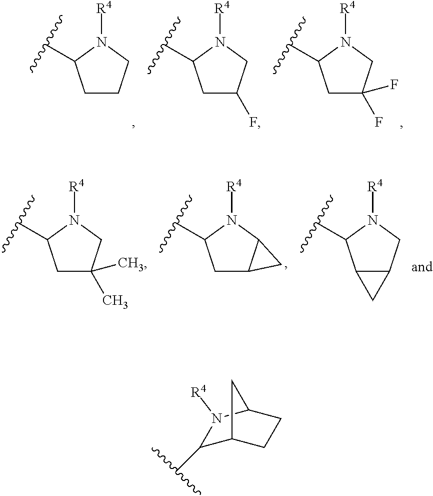

In still another embodiment, A and A′ are each independently selected from:

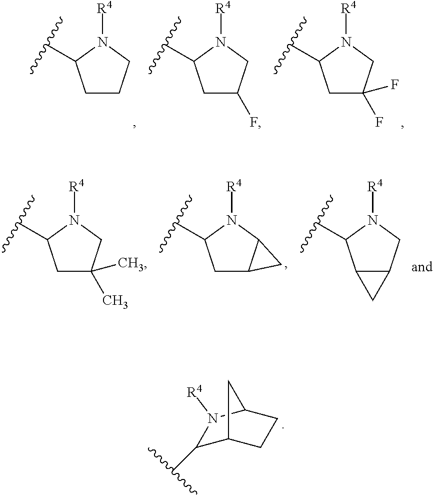

In another embodiment, A and A′ are each independently selected from:

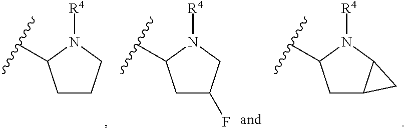

In another embodiment, A and A′ are each independently:

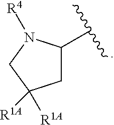

In another embodiment, A and A′ are each independently:

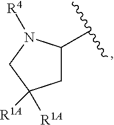

wherein each occurrence of R¹³ is independently H, CH₃, or F.

In one embodiment, each occurrence of R⁴ is independently:

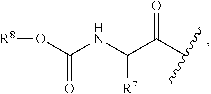

wherein R⁷ is selected from C₁-C₆ alkyl, C₁-C₆ haloalkyl and 4 to 6-membered monocyclic heterocycloalkyl, wherein said 4 to 6-membered monocyclic heterocycloalkyl group can be optionally substituted with up to five groups, each independently selected from halo, C₁-C₆ alkyl and C₃-C₇ cycloalkyl, and wherein said 4 to 6-membered monocyclic heterocycloalkyl group can be optionally substituted on a ring carbon atom with a spirocyclic C₃-C₆ cycloalkyl group; and R⁸ is C₁-C₆ alkyl.

In one embodiment, each occurrence of R⁴ is independently:

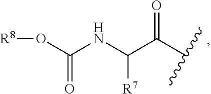

wherein R⁷ is selected from isopropyl, —CF(CH₃)₂,

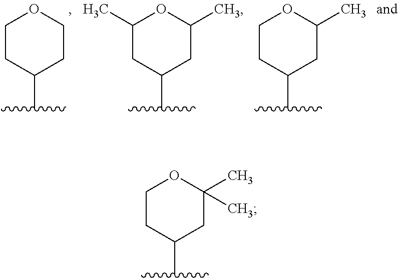

and R⁸ is C₁-C₆ alkyl.

In another embodiment, each occurrence of R⁴ is independently selected from:

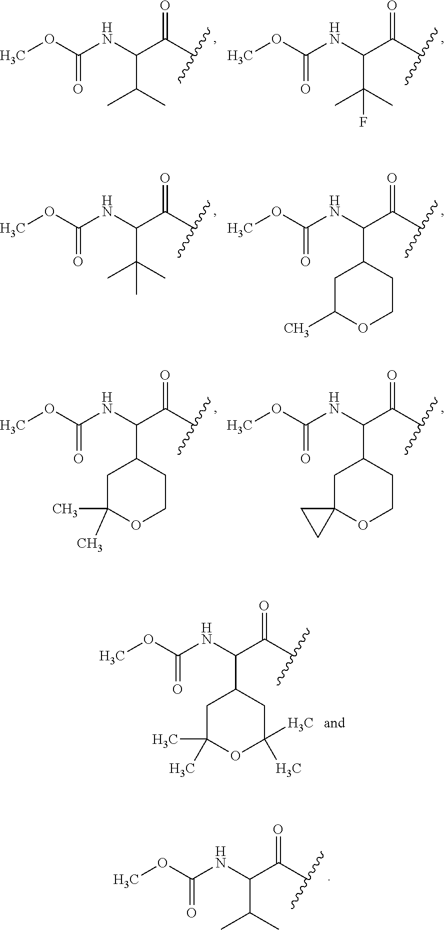

In one embodiment, A and A′ are each independently selected from:

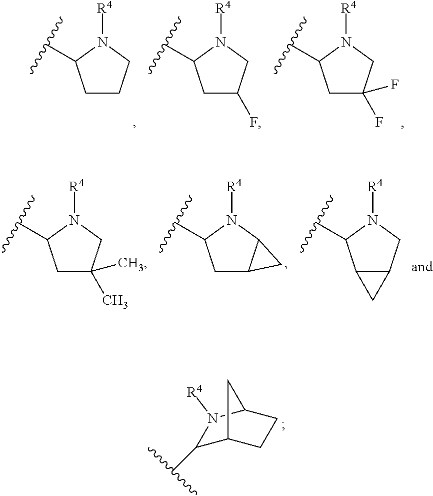

each occurrence of R⁴ is independently:

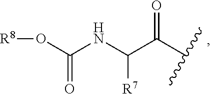

wherein R⁷ is selected from isopropyl, —CF(CH₃)₂,

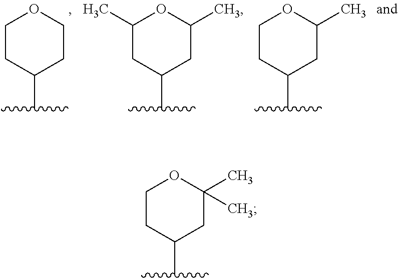

and R⁸ is C₁-C₆ alkyl.

In another embodiment, A and A′ are each independently selected from:

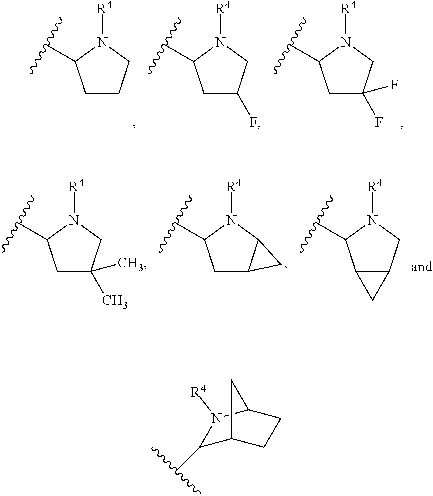

and R⁴ is:

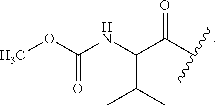 [SMILES: *C(=O)C(NC(=O)OC)C(C)C]

In yet another embodiment, A and A′ are each:

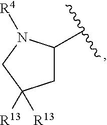

wherein each occurrence of R¹³ is independently H, CH₃, or F; each occurrence of R⁴ is independently:

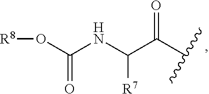

wherein R⁷ is selected from isopropyl, —CF(CH₃)₂,

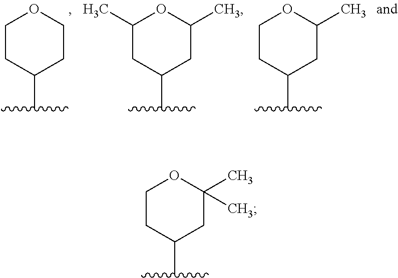

and R⁸ is C₁-C₆ alkyl.

In another embodiment, A and A′ are each independently selected from:

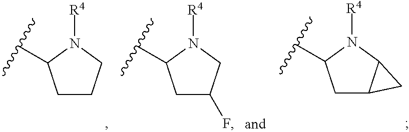

each occurrence of R⁴ is independently:

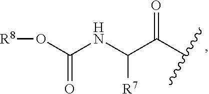

wherein R⁷ is selected from isopropyl, —CF(CH₃)₂,

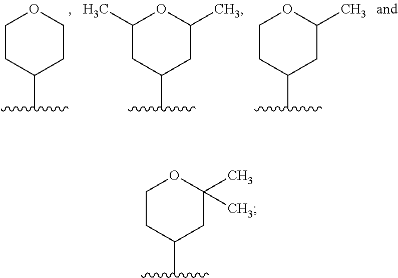

and R⁸ is methyl.

In one embodiment, variables A, A′, R², R³, R⁴ and R⁵ for the Compounds of Formula (I) are selected independently of each other.

In another embodiment, the Compounds of Formula (I) are in substantially purified form.

In one embodiment, the Compounds of Formula (I) have the formula (Ia):

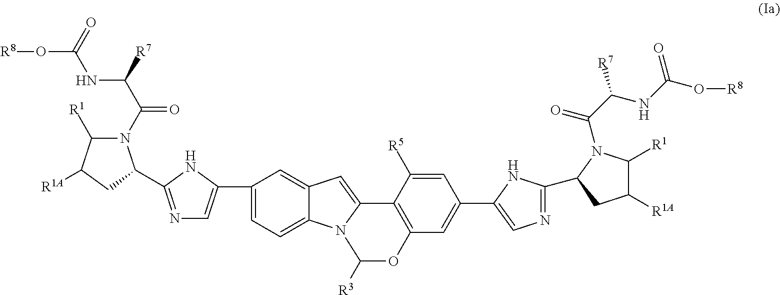

or a pharmaceutically acceptable salt thereof,
wherein:

each R¹ is H;

each R^{1A} is H, or an R^{1A} groups and an R¹ group that are attached to same ring, together with the ring carbon atoms to which they are attached, can combine to form a fused cyclopropyl group;

R³ is:

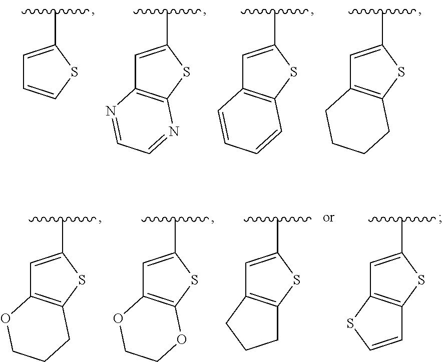

wherein R³ can be optionally substituted on one or more ring carbon atoms with a group selected from methyl, ethyl, n-propyl, isopropyl, t-butyl, F, —CHF₂, —CH₂CF₃, —CH₂F, —CF₃, cyclopropyl, cyclobutyl, cyclopentyl, —CH₂-cyclopropyl, methoxy, —O-(halo-substituted phenyl), —OCF₃, —C(CH₃)₂OH, —CH₂CH₂OCH₃, halo-substituted phenyl and —CN;

each occurrence of R⁵ is independently selected from H, methyl and F;

each occurrence of R⁷ is independently selected from C₁-C₆ alkyl, C₁-C₆ haloalkyl and 4 to 6-membered monocyclic heterocycloalkyl, wherein said 4 to 6-membered monocyclic heterocycloalkyl group can be optionally substituted with up to 5 groups, each independently selected from halo, C₁-C₆ alkyl and C₃-C₇ cycloalkyl, and wherein said 4 to 6-membered monocyclic heterocycloalkyl group can be optionally substituted on a ring carbon atom with a spirocyclic C₃-C₆ cycloalkyl group; each occurrence of R⁸ is independently C₁-C₆ alkyl.

In one embodiment, variables R′, R^{1A}, R³, R⁵, R⁷ and R⁸ for the Compounds of Formula (Ia) are selected independently of each other.

In another embodiment, the Compounds of Formula (Ia) are in substantially purified form.

In another embodiment, the Compounds of Formula (I) have the formula (Ib) or (Ic):

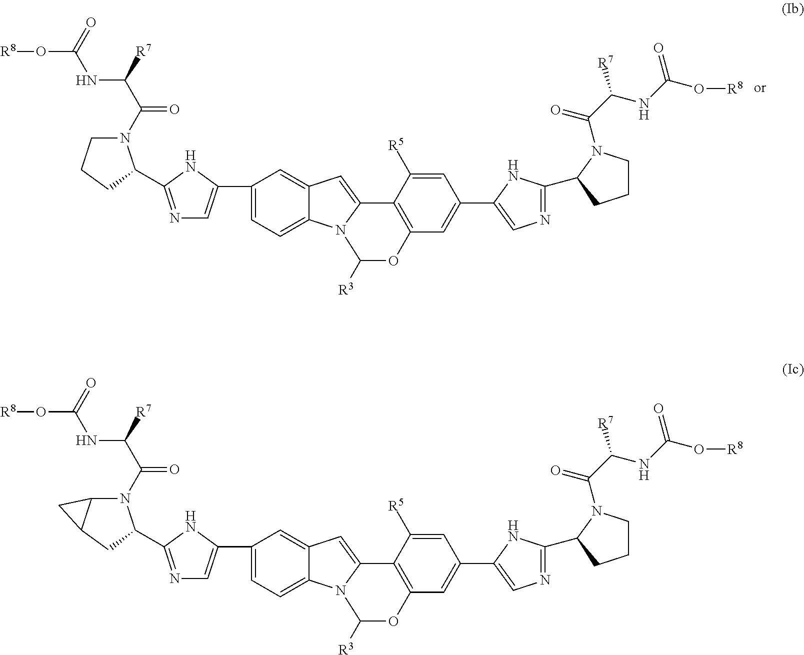

or a pharmaceutically acceptable salt thereof,
wherein:

R³ is:

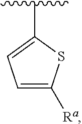

R^{a} is C₁-C₆ alkyl or C₃-C₇ cycloalkyl;

R⁵ is H or F;

each occurrence of R⁷ is independently selected from C₁-C₆ alkyl or tetrahydropyranyl, wherein said tetrahydropyranyl group can be optionally substituted with up to 5 groups, each independently selected from halo, C₃-C₇ cycloalkyl or C₁-C₆ alkyl, and wherein said tetrahydropyran group can be optionally substituted on a ring carbon atom with a spirocyclic cyclopropyl group.

each occurrence of R⁸ is methyl.

In one embodiment, for the compounds of formula (Ia), (Ib) or (Ic), each occurrence of R⁷ is selected from isopropyl, —CF(CH₃)₂,

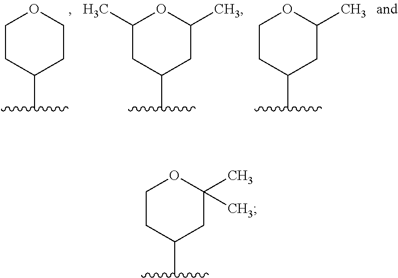

and each occurrence of R⁸ is methyl.

In one embodiment, for the compounds of formula (Ia), (Ib) or (Ic), R^{a} is cyclopropyl, ethyl, cyclopentyl, n-propyl, isopropyl or isobutyl

In another embodiment, for the compounds of formula (Ia), (Ib) or (Ic), R^{a} is cyclopropyl, ethyl, cyclopentyl, n-propyl, isopropyl, t-butyl or isobutyl; and R⁵ is F.

In another embodiment, for the compounds of formula (Ia), (Ib) or (Ic), R^{a} is cyclopropyl, ethyl, cyclopentyl, n-propyl, isopropyl, t-butyl or isobutyl; R⁵ is F; and each occurrence of R⁷ is independently isopropyl or —CF(CH₃)₂.

In still another embodiment, for the compounds of formula (Ia), (Ib) or (Ic), R^{a} is cyclopropyl or cyclobutyl; R⁵ is F; and each occurrence of R⁷ is independently isopropyl or —CF(CH₃)₂.

In one embodiment, variables R³, R⁵, R⁷ and R⁸ for the Compounds of Formula (Ib) are selected independently of each other.

In another embodiment, the Compounds of Formula (Ib) are in substantially purified form.

In one embodiment, variables R³, R⁵, R⁷ and R⁸ for the Compounds of Formula (Ic) are selected independently of each other.

In another embodiment, the Compounds of Formula (Ic) are in substantially purified form.

Other embodiments of the present invention include the following:

(a) A pharmaceutical composition comprising an effective amount of a Compound of Formula (I) or a pharmaceutically acceptable salt thereof, and a pharmaceutically acceptable carrier.

(b) The pharmaceutical composition of (a), further comprising a second therapeutic agent selected from the group consisting of HCV antiviral agents, immunomodulators, and anti-infective agents.

(c) The pharmaceutical composition of (b), wherein the HCV antiviral agent is an antiviral selected from the group consisting of HCV protease inhibitors and HCV NS5B polymerase inhibitors.

(d) A pharmaceutical combination that is (i) a Compound of Formula (I) and (ii) a second therapeutic agent selected from the group consisting of HCV antiviral agents, immunomodulators, and anti-infective agents; wherein the Compound of Formula (I) and the second therapeutic agent are each employed in an amount that renders the combination effective for inhibiting HCV replication, or for treating HCV infection and/or reducing the likelihood or severity of symptoms of HCV infection.

(e) The combination of (d), wherein the HCV antiviral agent is an antiviral selected from the group consisting of HCV protease inhibitors and HCV NS5B polymerase inhibitors.

(f) A method of inhibiting HCV replication in a subject in need thereof which comprises administering to the subject an effective amount of a Compound of Formula (I).

(g) A method of treating HCV infection and/or reducing the likelihood or severity of symptoms of HCV infection in a subject in need thereof which comprises administering to the subject an effective amount of a Compound of Formula (I).

(h) The method of (g), wherein the Compound of Formula (I) is administered in combination with an effective amount of at least one second therapeutic agent selected from the group consisting of HCV antiviral agents, immunomodulators, and anti-infective agents.

(i) The method of (h), wherein the HCV antiviral agent is an antiviral selected from the group consisting of HCV protease inhibitors and HCV NS5B polymerase inhibitors.

(j) A method of inhibiting HCV replication in a subject in need thereof which comprises administering to the subject the pharmaceutical composition of (a), (b) or (c) or the combination of (d) or (e).

(k) A method of treating HCV infection and/or reducing the likelihood or severity of symptoms of HCV infection in a subject in need thereof which comprises administering to the subject the pharmaceutical composition of (a), (b) or (c) or the combination of (d) or (e).

The present invention also includes a compound of the present invention for use (i) in, (ii) as a medicament for, or (iii) in the preparation of a medicament for: (a) medicine; (b) inhibiting HCV replication or (c) treating HCV infection and/or reducing the likelihood or severity of symptoms of HCV infection. In these uses, the compounds of the present invention can optionally be employed in combination with one or more second therapeutic agents selected from HCV antiviral agents, anti-infective agents, and immunomodulators.

Additional embodiments of the invention include the pharmaceutical compositions, combinations and methods set forth in (a)-(k) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate.

It is further to be understood that the embodiments of compositions and methods provided as (a) through (k) above are understood to include all embodiments of the compounds, including such embodiments as result from combinations of embodiments.

Non-limiting examples of the Compounds of Formula (I) include compounds 1-851, as set forth in the Examples below, and pharmaceutically acceptable salts thereof.

### Uses of the Thiophene-Substituted Tetr Acyclic Compounds

The Thiophene-Substituted Tetracyclic Compounds are useful in human and veterinary medicine for treating or preventing a viral infection in a patient. In one embodiment, the Thiophene-Substituted Tetracyclic Compounds can be inhibitors of viral replication. In another embodiment, the Thiophene-Substituted Tetracyclic Compounds can be inhibitors of HCV replication. Accordingly, the Thiophene-Substituted Tetracyclic Compounds are useful for treating viral infections, such as HCV. In accordance with the invention, the Thiophene-Substituted Tetracyclic Compounds can be administered to a patient in need of treatment or prevention of a viral infection.

Accordingly, in one embodiment, the invention provides methods for treating a viral infection in a patient comprising administering to the patient an effective amount of at least one Thiophene-Substituted Tetracyclic Compound or a pharmaceutically acceptable salt thereof.

### Treatment or Prevention of a Flaviviridae Virus

The Thiophene-Substituted Tetracyclic Compounds can be useful for treating or preventing a viral infection caused by the Flaviviridae family of viruses.

Examples of Flaviviridae infections that can be treated or prevented using the present methods include but are not limited to, dengue fever, Japanese encephalitis, Kyasanur Forest disease, Murray Valley encephalitis, St. Louis encephalitis, Tick-borne encephalitis, West Nile encephalitis, yellow fever and Hepatitis C Virus (HCV) infection.

In one embodiment, the Flaviviridae infection being treated is hepatitis C virus infection.

### Treatment or Prevention of HCV Infection

The Thiophene-Substituted Tetracyclic Compounds are useful in the inhibition of HCV replication, the treatment of HCV infection and/or reduction of the likelihood or severity of symptoms of HCV infection and the inhibition of HCV viral replication and/or HCV viral production in a cell-based system. For example, the Thiophene-Substituted Tetracyclic Compounds are useful in treating infection by HCV after suspected past exposure to HCV by such means as blood transfusion, exchange of body fluids, bites, accidental needle stick, or exposure to patient blood during surgery or other medical procedures.

In one embodiment, the hepatitis C infection is acute hepatitis C. In another embodiment, the hepatitis C infection is chronic hepatitis C.

Accordingly, in one embodiment, the invention provides methods for treating HCV infection in a patient, the methods comprising administering to the patient an effective amount of at least one Thiophene-Substituted Tetracyclic Compound or a pharmaceutically acceptable salt thereof. In a specific embodiment, the amount administered is effective to treat or prevent infection by HCV in the patient. In another specific embodiment, the amount administered is effective to inhibit HCV viral replication and/or viral production in the patient.

The Thiophene-Substituted Tetracyclic Compounds are also useful in the preparation and execution of screening assays for antiviral compounds. For example the Thiophene-Substituted Tetracyclic Compounds are useful for identifying resistant HCV replicon cell lines harboring mutations within NS5A, which are excellent screening tools for more powerful antiviral compounds. Furthermore, the Thiophene-Substituted Tetracyclic Compounds are useful in establishing or determining the binding site of other antivirals to the HCV replicase.

The compositions and combinations of the present invention can be useful for inhibiting different HCV genotypes. HCV types and subtypes may differ in their antigenicity, level of viremia, severity of disease produced, and response to interferon therapy as described in Holland et al., Pathology, 30(2):192-195 (1998). The nomenclature set forth in Simmonds et al., J Gen Virol, 74(Pt11):2391-2399 (1993) is widely used and classifies isolates into six major genotypes, 1 through 6, with two or more related subtypes, e.g., 1a and 1b. Additional genotypes 7-10 and 11 have been proposed, however the phylogenetic basis on which this classification is based has been questioned, and thus types 7, 8, 9 and 11 isolates have been reassigned as type 6, and type 10 isolates as type 3 (see Lamballerie et al., J Gen Virol, 78(Pt1):45-51 (1997)). The major genotypes have been defined as having sequence similarities of between 55 and 72% (mean 64.5%), and subtypes within types as having 75%-86% similarity (mean 80%) when sequenced in the NS-5 region (see Simmonds et al., J Gen Virol, 75(Pt 5):1053-1061 (1994)).

### Combination Therapy

In another embodiment, the present methods for treating or preventing HCV infection can further comprise the administration of one or more additional therapeutic agents which are not Thiophene-Substituted Tetracyclic Compounds.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a patient, the method comprising administering to the patient: (i) at least one Thiophene-Substituted Tetracyclic Compound, or a pharmaceutically acceptable salt thereof, and (ii) at least one additional therapeutic agent that is other than a Thiophene-Substituted Tetracyclic Compound, wherein the amounts administered are together effective to treat or prevent a viral infection.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Thiophene-Substituted Tetracyclic Compound and an additional therapeutic agent may be present in fixed amounts (dosage amounts) in a single dosage unit (e.g., a capsule, a tablet and the like).

In one embodiment, the at least one Thiophene-Substituted Tetracyclic Compound is administered during a time when the additional therapeutic agent(s) exert their prophylactic or therapeutic effect, or vice versa.

In another embodiment, the at least one Thiophene-Substituted Tetracyclic Compound and the additional therapeutic agent(s) are administered in doses commonly employed when such agents are used as monotherapy for treating a viral infection.

In another embodiment, the at least one Thiophene-Substituted Tetracyclic Compound and the additional therapeutic agent(s) are administered in doses lower than the doses commonly employed when such agents are used as monotherapy for treating a viral infection.

In still another embodiment, the at least one Thiophene-Substituted Tetracyclic Compound and the additional therapeutic agent(s) act synergistically and are administered in doses lower than the doses commonly employed when such agents are used as monotherapy for treating a viral infection.

In one embodiment, the at least one Thiophene-Substituted Tetracyclic Compound and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

In one embodiment, the viral infection is HCV infection.

The at least one Thiophene-Substituted Tetracyclic Compound and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Thiophene-Substituted Tetracyclic Compound and the additional therapeutic agent(s) may inhibit the resistance of a viral infection to these agents.

Non-limiting examples of additional therapeutic agents useful in the present compositions and methods include an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.

In one embodiment, the additional therapeutic agent is a viral protease inhibitor.

In another embodiment, the additional therapeutic agent is a viral replication inhibitor.

In another embodiment, the additional therapeutic agent is an HCV NS3 protease inhibitor.

In still another embodiment, the additional therapeutic agent is an HCV NS5B polymerase inhibitor.

In another embodiment, the additional therapeutic agent is a nucleoside inhibitor.

In another embodiment, the additional therapeutic agent is an interferon.

In yet another embodiment, the additional therapeutic agent is an HCV replicase inhibitor.

In another embodiment, the additional therapeutic agent is an antisense agent.

In another embodiment, the additional therapeutic agent is a therapeutic vaccine.

In a further embodiment, the additional therapeutic agent is a virion production inhibitor.

In another embodiment, the additional therapeutic agent is an antibody therapy.

In another embodiment, the additional therapeutic agent is an HCV NS2 inhibitor.

In still another embodiment, the additional therapeutic agent is an HCV NS4A inhibitor.

In another embodiment, the additional therapeutic agent is an HCV NS4B inhibitor.

In another embodiment, the additional therapeutic agent is an HCV NS5A inhibitor

In yet another embodiment, the additional therapeutic agent is an HCV NS3 helicase inhibitor.

In another embodiment, the additional therapeutic agent is an HCV IRES inhibitor.

In another embodiment, the additional therapeutic agent is an HCV p7 inhibitor.

In a further embodiment, the additional therapeutic agent is an HCV entry inhibitor.

In another embodiment, the additional therapeutic agent is an HCV assembly inhibitor.

In one embodiment, the additional therapeutic agents comprise a viral protease inhibitor and a viral polymerase inhibitor.

In still another embodiment, the additional therapeutic agents comprise a viral protease inhibitor and an immunomodulatory agent.

In yet another embodiment, the additional therapeutic agents comprise a polymerase inhibitor and an immunomodulatory agent.

In another embodiment, the additional therapeutic agents comprise a viral protease inhibitor and a nucleoside.

In another embodiment, the additional therapeutic agents comprise an immunomodulatory agent and a nucleoside.

In one embodiment, the additional therapeutic agents comprise an HCV protease inhibitor and an HCV polymerase inhibitor.

In another embodiment, the additional therapeutic agents comprise a nucleoside and an HCV NS5A inhibitor.

In another embodiment, the additional therapeutic agents comprise a viral protease inhibitor, an immunomodulatory agent and a nucleoside.

In a further embodiment, the additional therapeutic agents comprise a viral protease inhibitor, a viral polymerase inhibitor and an immunomodulatory agent.

In another embodiment, the additional therapeutic agent is ribavirin.

HCV polymerase inhibitors useful in the present compositions and methods include, but are not limited to, VP-19744 (Wyeth/ViroPharma), PSI-7851 (Pharmasset), GS-7977 (sofosbuvir, Gilead), R7128 (Roche/Pharmasset), PF-868554/filibuvir (Pfizer), VCH-759 (ViroChem Pharma), HCV-796 (Wyeth/ViroPharma), IDX-184 (Idenix), IDX-375 (Idenix), NM-283 (Idenix/Novartis), R-1626 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3281 (Merck), VCH222 (ViroChem), VCH916 (ViroChem), VCH716(ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004).

Other HCV polymerase inhibitors useful in the present compositions and methods include, but are not limited to, those disclosed in International Publication Nos. WO 08/082484, WO 08/082488, WO 08/083351, WO 08/136815, WO 09/032116, WO 09/032123, WO 09/032124 and WO 09/032125.

Interferons useful in the present compositions and methods include, but are not limited to, interferon alfa-2a, interferon alfa-2b, interferon alfacon-1 and PEG-interferon alpha conjugates. “PEG-interferon alpha conjugates” are interferon alpha molecules covalently attached to a PEG molecule. Illustrative PEG-interferon alpha conjugates include interferon alpha-2a (Roferon™, Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-Intron™ from Schering-Plough Corporation), interferon alpha-2b-XL (e.g., as sold under the trade name PEG-Intron™), interferon alpha-2c (Berofor Alpha™, Boehringer Ingelheim, Ingelheim, Germany), PEG-interferon lambda (Bristol-Myers Squibb and ZymoGenetics), interferon alfa-2b alpha fusion polypeptides, interferon fused with the human blood protein albumin (Albuferon™, Human Genome Sciences), Omega Interferon (Intarcia), Locteron controlled release interferon (Biolex/OctoPlus), Biomed-510 (omega interferon), Peg-IL-29 (ZymoGenetics), Locteron CR (Octoplus), IFN-α-2b-XL (Flamel Technologies), and consensus interferon as defined by determination of a consensus sequence of naturally occurring interferon alphas (Infergen™, Amgen, Thousand Oaks, Calif.).

Antibody therapy agents useful in the present compositions and methods include, but are not limited to, antibodies specific to IL-10 (such as those disclosed in US Patent Publication No. US2005/0101770, humanized 12G8, a humanized monoclonal antibody against human IL-10, plasmids containing the nucleic acids encoding the humanized 12G8 light and heavy chains were deposited with the American Type Culture Collection (ATCC) as deposit numbers PTA-5923 and PTA-5922, respectively), and the like).

Examples of viral protease inhibitors useful in the present compositions and methods include, but are not limited to, an HCV protease inhibitor.

HCV protease inhibitors useful in the present compositions and methods include, but are not limited to, those disclosed in U.S. Pat. Nos. 7,494,988, 7,485,625, 7,449,447, 7,442,695, 7,425,576, 7,342,041, 7,253,160, 7,244,721, 7,205,330, 7,192,957, 7,186,747, 7,173,057, 7,169,760, 7,012,066, 6,914,122, 6,911,428, 6,894,072, 6,846,802, 6,838,475, 6,800,434, 6,767,991, 5,017,380, 4,933,443, 4,812,561 and 4,634,697; U.S. Patent Publication Nos. US20020068702, US20020160962, US20050119168, US20050176648, US20050209164, US20050249702 and US20070042968; and International Publication Nos. WO 03/006490, WO 03/087092, WO 04/092161 and WO 08/124148.

Additional HCV protease inhibitors useful in the present compositions and methods include, but are not limited to, SCH503034 (Boceprevir, Schering-Plough), SCH900518 (Schering-Plough), VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott), TMC-435350 (Medivir), ITMN-191/R7227 (InterMune/Roche), EA-058 (Abbott/Enanta), EA-063 (Abbott/Enanta), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex) and PHX1766 (Phenomix).

Further examples of HCV protease inhibitors useful in the present compositions and methods include, but are not limited to, those disclosed in Landro et al., Biochemistry, 36(31):9340-9348 (1997); Ingallinella et al., Biochemistry, 37(25):8906-8914 (1998); Llinás-Brunet et al., Bioorg Med Chem Lett, 8(13):1713-1718 (1998); Martin et al., Biochemistry, 37(33):11459-11468 (1998); Dimasi et al., J Virol, 71(10):7461-7469 (1997); Martin et al., Protein Eng, 10(5):607-614 (1997); Elzouki et al., J Hepat, 27(1):42-48 (1997); BioWorld Today, 9(217):4 (Nov. 10, 1998); U.S. Patent Publication Nos. US2005/0249702 and US 2007/0274951; and International Publication Nos. WO 98/14181, WO 98/17679, WO 98/17679, WO 98/22496 and WO 99/07734 and WO 05/087731.

Further examples of HCV protease inhibitors useful in the present compositions and methods include, but are not limited to, MK-5172 (Merck) and the following compounds:

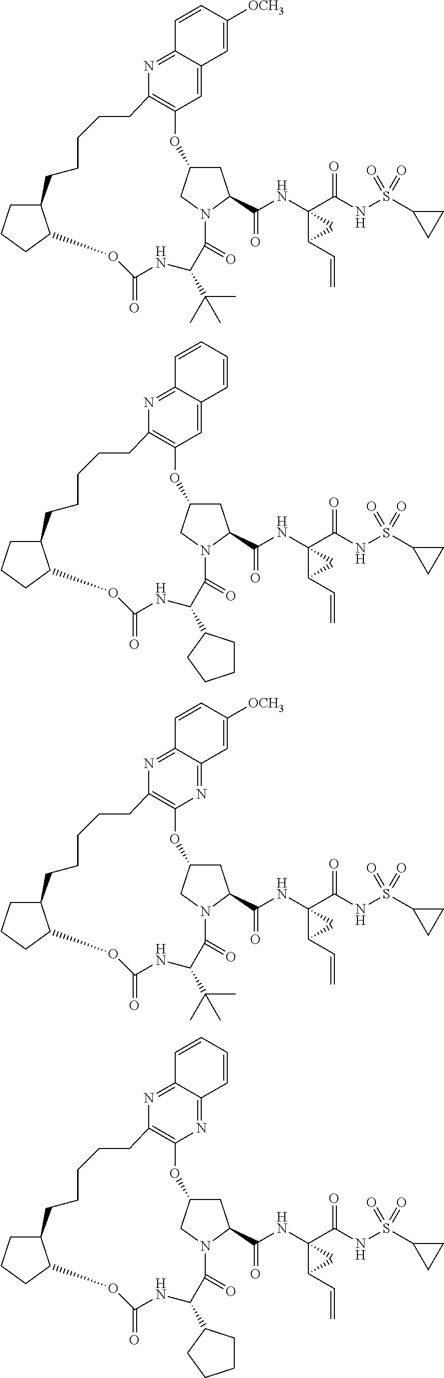

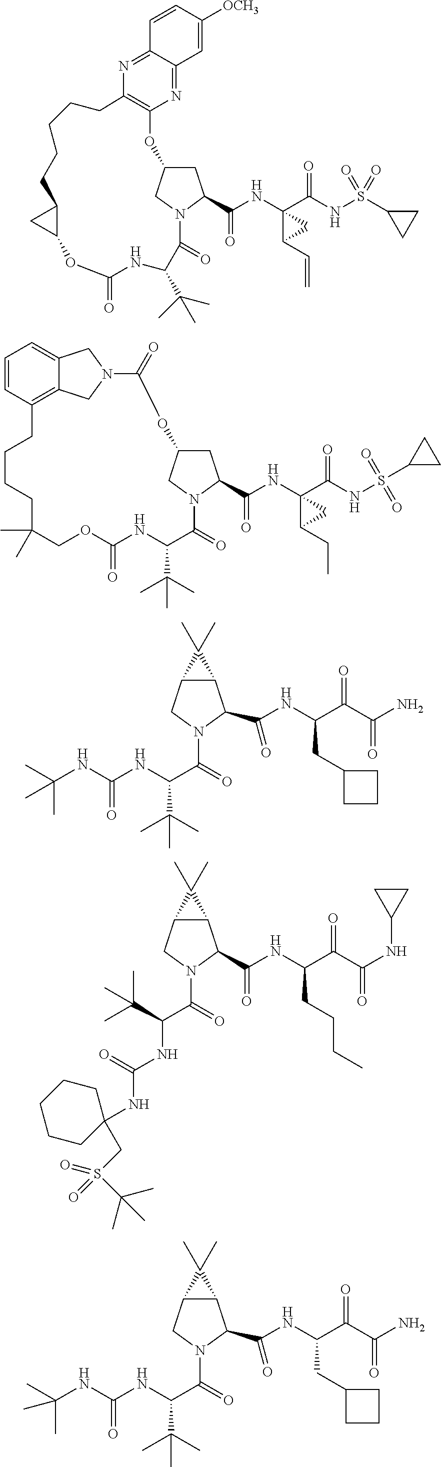

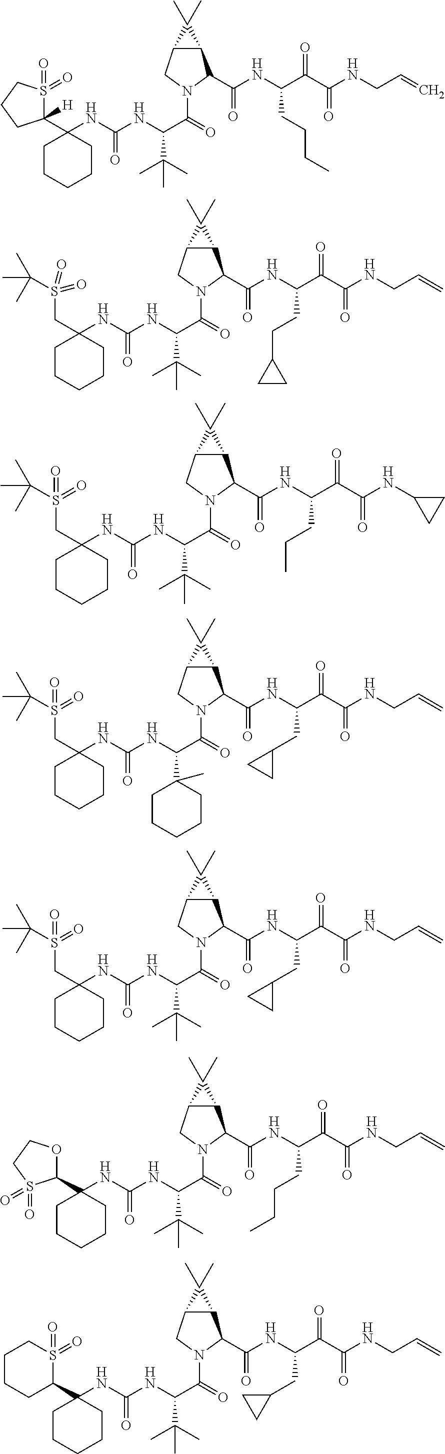

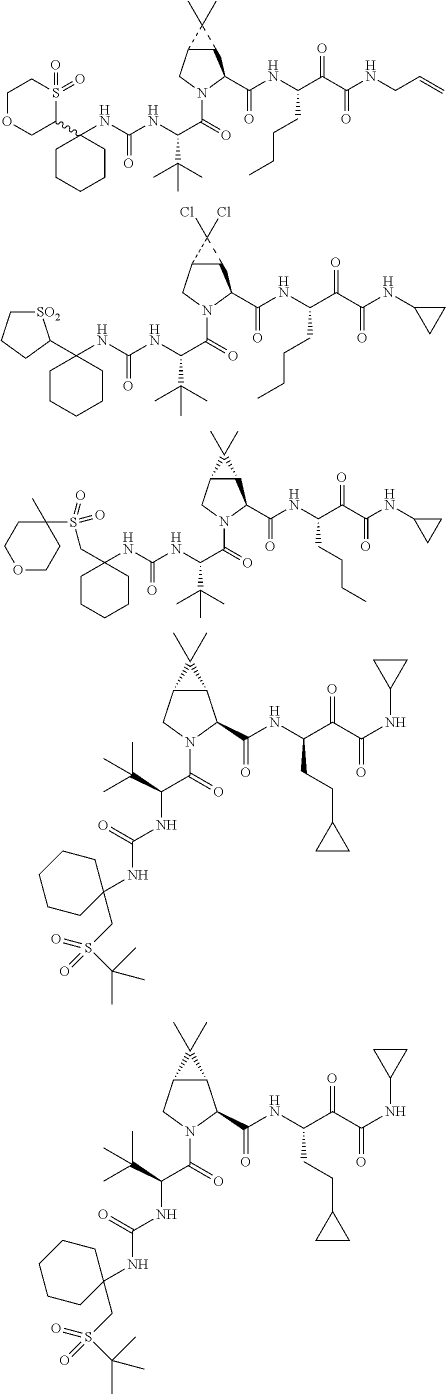

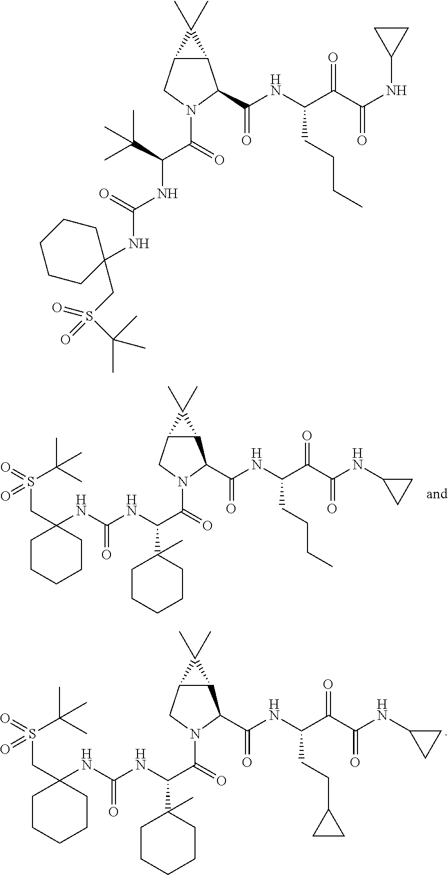

HCV viral replication inhibitors useful in the present compositions and methods include, but are not limited to, HCV replicase inhibitors, IRES inhibitors, NS4A inhibitors, NS3 helicase inhibitors, NS3 protease inhibitors, NS5A inhibitors, NS5B inhibitors, ribavirin, AZD-2836 (Astra Zeneca), BMS-790052 (Bristol-Myers Squibb, see Gao et al., Nature, 465:96-100 (2010)), viramidine, A-831 (Arrow Therapeutics); an antisense agent or a therapeutic vaccine.

HCV NS4A inhibitors useful in the useful in the present compositions and methods include, but are not limited to, those disclosed in U.S. Pat. Nos. 7,476,686 and 7,273,885; U.S. Patent Publication No. US20090022688; and International Publication Nos. WO 2006/019831 and WO 2006/019832. Additional HCV NS4A inhibitors useful in the useful in the present compositions and methods include, but are not limited to, AZD2836 (Astra Zeneca) and ACH-806 (Achillon Pharmaceuticals, New Haven, Conn.).

HCV replicase inhibitors useful in the useful in the present compositions and methods include, but are not limited to, those disclosed in U.S. Patent Publication No. US20090081636.

Therapeutic vaccines useful in the present compositions and methods include, but are not limited to, IC41 (Intercell Novartis), CSL123 (Chiron/CSL), GI 5005 (Globeimmune), TG-4040 (Transgene), GNI-103 (GENimmune), Hepavaxx C (ViRex Medical), ChronVac-C (Inovio/Tripep), PeviPRO™ (Pevion Biotect), HCV/MF59 (Chiron/Novartis) and Civacir (NABI).

Examples of further additional therapeutic agents useful in the present compositions and methods include, but are not limited to, Ritonavir (Abbott), TT033 (Benitec/Tacere Bio/Pfizer), Sirna-034 (Sirna Therapeutics), GNI-104 (GENimmune), GI-5005 (GlobeImmune), IDX-102 (Idenix), Levovirin™ (ICN Pharmaceuticals, Costa Mesa, Calif.); Humax (Genmab), ITX-2155 (Ithrex/Novartis), PRO 206 (Progenics), HepaCide-I (NanoVirocides), MX3235 (Migenix), SCY-635 (Scynexis); KPE02003002 (Kemin Pharma), Lenocta (VioQuest Pharmaceuticals), IET—Interferon Enhancing Therapy (Transition Therapeutics), Zadaxin (SciClone Pharma), VP 50406™ (Viropharma, Incorporated, Exton, Pa.); Taribavirin (Valeant Pharmaceuticals); Nitazoxanide (Romark); Debio 025 (Debiopharm); GS-9450 (Gilead); PF-4878691 (Pfizer); ANA773 (Anadys); SCV-07 (SciClone Pharmaceuticals); NIM-881 (Novartis); ISIS 14803™ (ISIS Pharmaceuticals, Carlsbad, Calif.); Heptazyme™ (Ribozyme Pharmaceuticals, Boulder, Colo.); Thymosin™ (SciClone Pharmaceuticals, San Mateo, Calif.); Maxamine™ (Maxim Pharmaceuticals, San Diego, Calif.); NKB-122 (JenKen Bioscience Inc., North Carolina); Alinia (Romark Laboratories), INFORM-1 (a combination of R7128 and ITMN-191); and mycophenolate mofetil (Hoffman-LaRoche, Nutley, N.J.)

The doses and dosage regimen of the other agents used in the combination therapies of the present invention for the treatment or prevention of HCV infection can be determined by the attending clinician, taking into consideration the approved doses and dosage regimen in the package insert; the age, sex and general health of the patient; and the type and severity of the viral infection or related disease or disorder. When administered in combination, the Thiophene-Substituted Tetracyclic Compound(s) and the other agent(s) can be administered simultaneously (i.e., in the same composition or in separate compositions one right after the other) or sequentially. This particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms is therefore advantageous.

In a further embodiment, when the additional therapeutic agent is Ribavirin (commercially available as REBETOL ribavirin from Schering-Plough or COPEGUS ribavirin from Hoffmann-La Roche), this agent is administered at a daily dosage of from about 600 to about 1400 mg/day for at least 24 weeks.

In one embodiment, one or more compounds of the present invention are administered with one or more additional therapeutic agents selected from: an immunomodulator, a viral replication inhibitor, an antisense agent, a therapeutic vaccine, a viral polymerase inhibitor, a nucleoside inhibitor, a viral protease inhibitor, a viral helicase inhibitor, a viral polymerase inhibitor a virion production inhibitor, a viral entry inhibitor, a viral assembly inhibitor, an antibody therapy (monoclonal or polyclonal), and any agent useful for treating an RNA-dependent polymerase-related disorder.

In another embodiment, one or more compounds of the present invention are administered with one or more additional therapeutic agents selected from an HCV protease inhibitor, an HCV polymerase inhibitor, an HCV replication inhibitor, a nucleoside and ribavirin. The combination therapies can include any combination of these additional therapeutic agents.

In another embodiment, one or more compounds of the present invention are administered with one additional therapeutic agent selected from an HCV protease inhibitor and ribavirin.

In still another embodiment, one or more compounds of the present invention are administered with two additional therapeutic agents selected from an HCV protease inhibitor, an HCV replication inhibitor, a nucleoside and ribavirin.

In another embodiment, one or more compounds of the present invention are administered with an HCV protease inhibitor and ribavirin. In another specific embodiment, one or more compounds of the present invention are administered with ribavirin.

In another embodiment, one or more compounds of the present invention are administered with three additional therapeutic agents selected from an HCV protease inhibitor, an HCV replication inhibitor, a nucleoside, a pegylated interferon and ribavirin.

In one embodiment, one or more compounds of the present invention are administered with one or more additional therapeutic agents selected from an HCV polymerase inhibitor, a viral protease inhibitor, and a viral replication inhibitor. In another embodiment, one or more compounds of the present invention are administered with one or more additional therapeutic agents selected from an HCV polymerase inhibitor, a viral protease inhibitor, and a viral replication inhibitor. In another embodiment, one or more compounds of the present invention are administered with one or more additional therapeutic agents selected from an HCV polymerase inhibitor, a viral protease inhibitor, and ribavirin.

In one embodiment, one or more compounds of the present invention are administered with one additional therapeutic agent selected from an HCV polymerase inhibitor, a viral protease inhibitor, and a viral replication inhibitor. In another embodiment, one or more compounds of the present invention are administered with ribavirin.

In one embodiment, one or more compounds of the present invention are administered with two additional therapeutic agents selected from an HCV polymerase inhibitor, a viral protease inhibitor, and a viral replication inhibitor.

In another embodiment, one or more compounds of the present invention are administered with ribavirin and another therapeutic agent.

In another embodiment, one or more compounds of the present invention are administered with ribavirin and another therapeutic agent, wherein the additional therapeutic agent is selected from an HCV polymerase inhibitor, a viral protease inhibitor, and a viral replication inhibitor.

In still another embodiment, one or more compounds of the present invention are administered with ribavirin and a viral protease inhibitor.

In another embodiment, one or more compounds of the present invention are administered with ribavirin and an HCV protease inhibitor.

In another embodiment, one or more compounds of the present invention are administered with ribavirin and either boceprevir or telaprevir.

In a further embodiment, one or more compounds of the present invention are administered with ribavirin and an HCV polymerase inhibitor.

In another embodiment, one or more compounds of the present invention are administered with ribavirin.

In one embodiment, one or more compounds of the present invention are administered with MK-5172.

In one embodiment, one or more compounds of the present invention are administered with sofosbuvir.

### Compositions and Administration

Due to their activity, the Thiophene-Substituted Tetracyclic Compounds are useful in veterinary and human medicine. As described above, the Thiophene-Substituted Tetracyclic Compounds are useful for treating or preventing HCV infection in a patient in need thereof.

When administered to a patient, the Thiophene-Substituted Tetracyclic Compounds can be administered as a component of a composition that comprises a pharmaceutically acceptable carrier or vehicle. The present invention provides pharmaceutical compositions comprising an effective amount of at least one Thiophene-Substituted Tetracyclic Compound and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Thiophene-Substituted Tetracyclic Compounds are administered orally.

In another embodiment, the one or more Thiophene-Substituted Tetracyclic Compounds are administered intravenously.

In still another embodiment, the one or more Thiophene-Substituted Tetracyclic Compounds are administered sublingually.

In one embodiment, a pharmaceutical preparation comprising at least one Thiophene-Substituted Tetracyclic Compound is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the Thiophene-Substituted Tetracyclic Compound(s) by weight or volume. In various embodiments, the present compositions can contain, in one embodiment, from about 1% to about 70% or from about 5% to about 60% of the Thiophene-Substituted Tetracyclic Compound(s) by weight or volume.

The amount and frequency of administration of the Thiophene-Substituted Tetracyclic Compounds will be regulated according to the judgment of the attending clinician considering such factors as age, condition and size of the patient as well as severity of the symptoms being treated. Generally, a total daily dosage of the at least one Thiophene-Substituted Tetracyclic Compound(s) alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) at least one Thiophene-Substituted Tetracyclic Compound or a pharmaceutically acceptable salt thereof; (ii) one or more additional therapeutic agents that are not a Thiophene-Substituted Tetracyclic Compound; and (iii) a pharmaceutically acceptable carrier, wherein the amounts in the composition are together effective to treat HCV infection.

In one embodiment, the present invention provides compositions comprising a Compound of Formula (I) and a pharmaceutically acceptable carrier.

In another embodiment, the present invention provides compositions comprising a Compound of Formula (I), a pharmaceutically acceptable carrier, and a second therapeutic agent selected from the group consisting of HCV antiviral agents, immunomodulators, and anti-infective agents.

In another embodiment, the present invention provides compositions comprising a Compound of Formula (I), a pharmaceutically acceptable carrier, and to additional therapeutic agents, each of which are independently selected from the group consisting of HCV antiviral agents, immunomodulators, and anti-infective agents.

### Kits

In one aspect, the present invention provides a kit comprising a therapeutically effective amount of at least one Thiophene-Substituted Tetracyclic Compound, or a pharmaceutically acceptable salt, solvate, ester or prodrug of said compound and a pharmaceutically acceptable carrier, vehicle or diluent.

In another aspect the present invention provides a kit comprising an amount of at least one Thiophene-Substituted Tetracyclic Compound, or a pharmaceutically acceptable salt, solvate, ester or prodrug of said compound and an amount of at least one additional therapeutic agent listed above, wherein the amounts of the two or more active ingredients result in a desired therapeutic effect. In one embodiment, the one or more Thiophene-Substituted Tetracyclic Compounds and the one or more additional therapeutic agents are provided in the same container. In one embodiment, the one or more Thiophene-Substituted Tetracyclic Compounds and the one or more additional therapeutic agents are provided in separate containers.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized in Schemes 1-4 below. Alternative synthetic pathways and analogous structures will be apparent to those skilled in the art of organic synthesis.

Scheme 1 shows methods useful for making the compounds of formula G3, which are useful intermediates for making the Compounds of Formula (I).

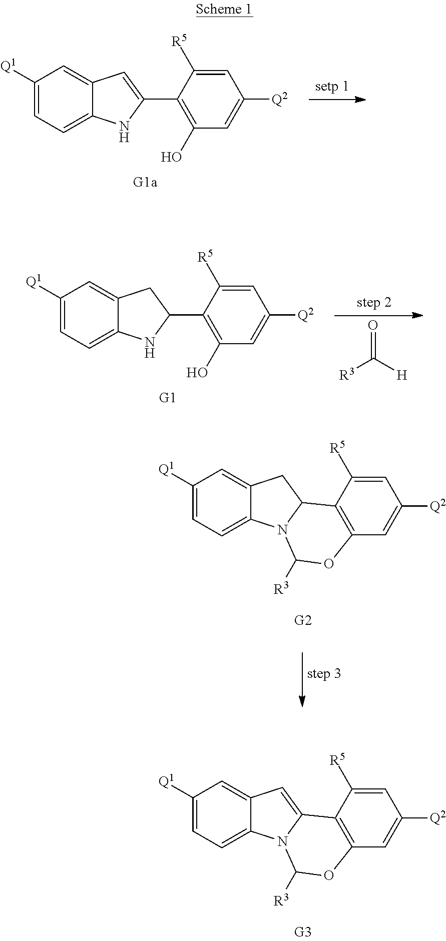

Wherein R³ and R⁵ are defined above for the Compounds of Formula (I) and Q¹ and Q² are each independently halo, hydroxyl, or a protected hydroxyl group, such as a methoxy or benzyloxy group.

An indole compound of formula G1a (which can be prepared as described in International Publication No. WO 2012/040923) can be treated with tin in conc.HCl/EtOH solution to provide compounds of formula G1. A compound of formula G1 can be reacted with an aldehyde of formula R³CHO in the presence of an acid to provide tetracyclic compounds of formula G2. Compounds of formula G2 can then be oxidized to provide the tetracyclic compounds of formula G3.

Scheme 2 shows methods useful for making the compounds of formula G5, which are useful intermediates for making the Compounds of Formula (I).

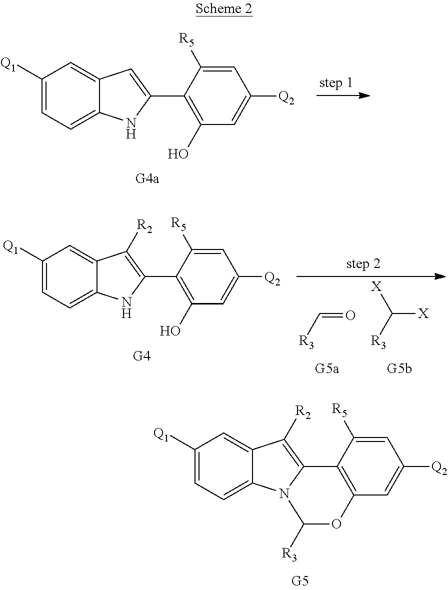

Wherein R², R³ and R⁵ are defined above for the Compounds of Formula (I), X is halo, and Q¹ and Q² are each independently halo, hydroxyl, or a protected hydroxyl group, such as a methoxy or benzyloxy group.

A compound of formula G4a (which can be prepared as described in International Publication No. WO 2012/040923) can be halogenated to provide the compounds of formula G4. A compounds of formula G4 can then be converted to the compounds of formula G5 via reaction with an aldehyde of formula G5a in the presence of an acid, or alternatively, by reaction with a dihalo compound of formula G5b in the presence of a base.

Scheme 3 shows methods useful for making the compounds of formula G12, which are useful intermediates for making the Compounds of Formula (I).

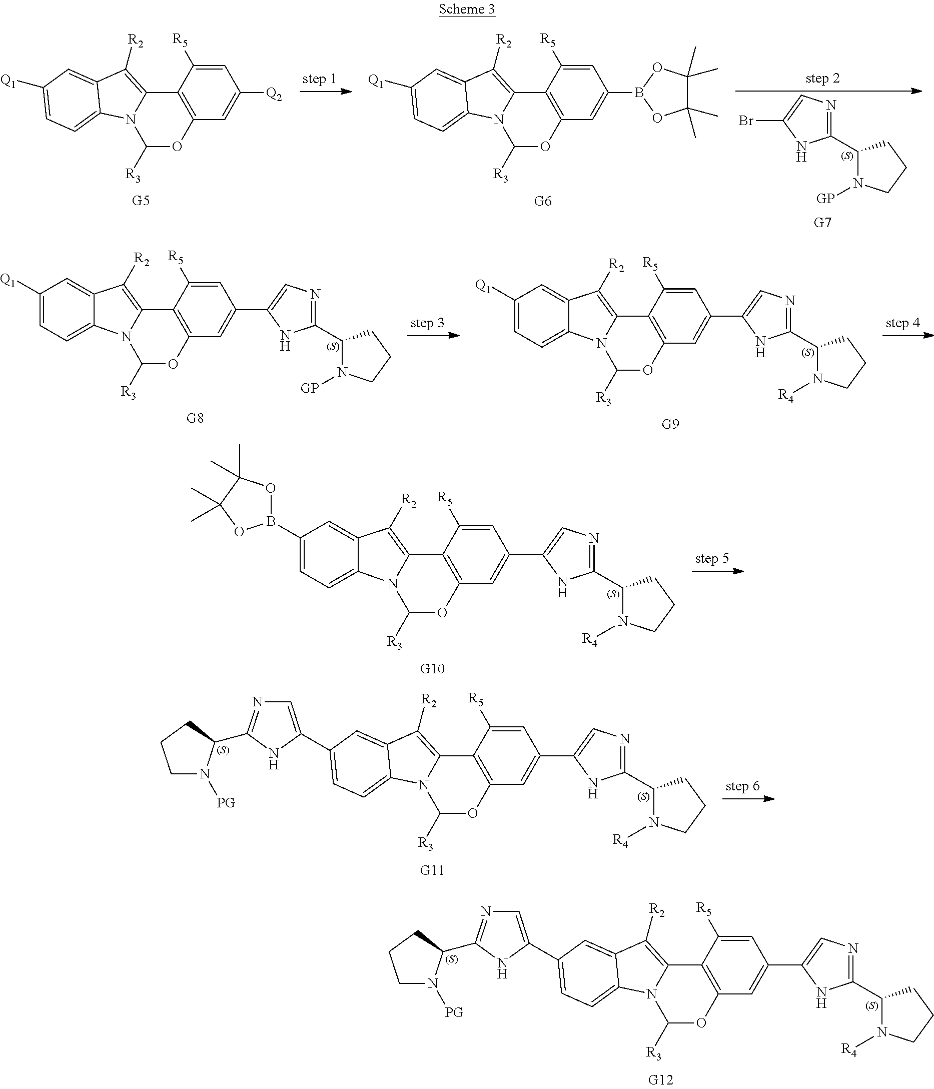

Wherein R², R³, R⁴ and R⁵ are defined above for the Compounds of Formula (I), PG is a secondary amino protecting group, and Q¹ and Q² are each independently halo, hydroxyl, or a protected hydroxyl group, such as a methoxy or benzyloxy group.

A compound of formula G5 can be reacted with bis(pinacolato)diboron to provide the compounds of formula G6. A compound of formula G6 can then undergo a Pd-mediated coupling with a bromo compound of formula G7 (prepared as described in International Publication No. WO 2012/040923) to provide the compounds of formula G8. Compounds of formula G8 can then be deprotected and subjected to an amide coupling with a desired cap compound to provide a compound of formula G9. A compound of formula G9 is then subjected to a Pd-mediated coupling with bis(pinacolato)diboron to provide the boronic ester compounds of formula G10. A compound of formula G10 can then undergo a Pd-mediated coupling with a bromo compound of formula G7 (prepared as described in International Publication No. WO 2012/040923) to provide the compounds of formula G11. Compounds of formula G11 can then be deprotected and subjected to an amide coupling with a desired cap compound to provide a compound of formula G12. Distereoisomers of the synthetic intermediates and final products can be separated using SFC or HPLC with chiral columns.

Scheme 4 shows methods useful for making the compounds of formula G18, which correspond to the Compounds of Formula (I).

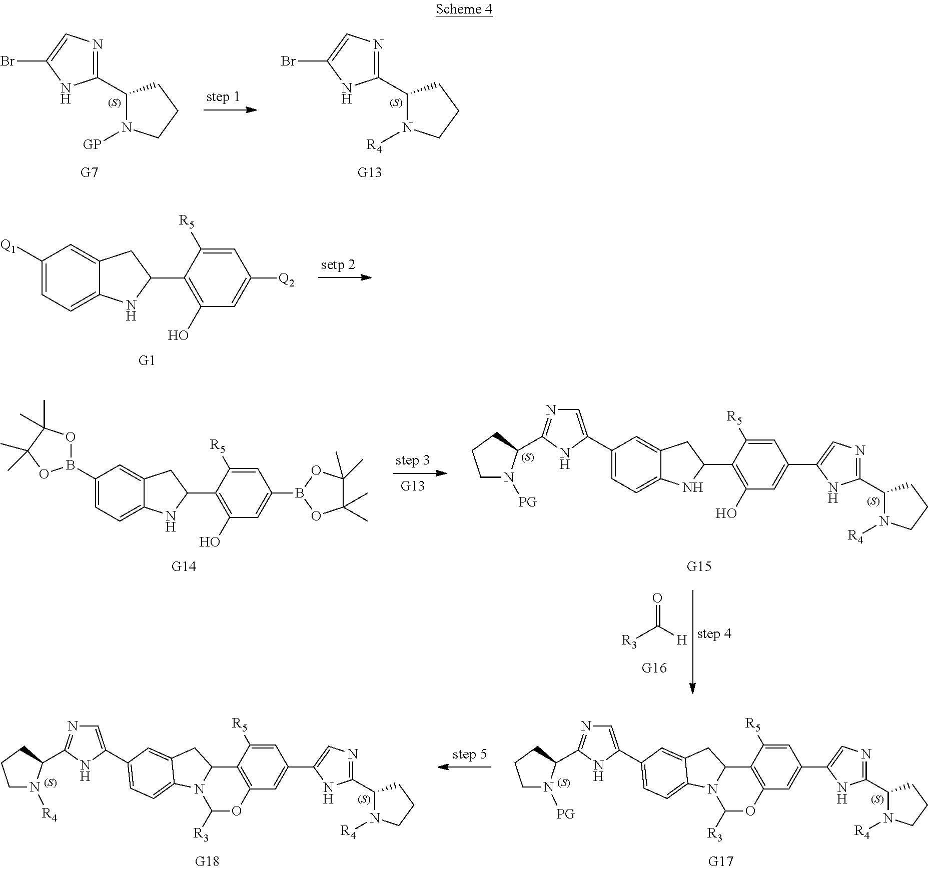

Wherein R³, R⁴ and R⁵ are defined above for the Compounds of Formula (I), PG is a secondary amino protecting group, and Q¹ and Q² are each independently halo, hydroxyl, or a protected hydroxyl group, such as a methoxy or benzyloxy group.

A compounds of formula G7 can then be deprotected and subjected to an amide coupling with a desired cap compound to provide a compound of formula G12. A compound of formula G1 can be converted to compound of formula G14 via a Pd mediated coupling reaction with bis(pinacolato)diboron. A compound of formula G14 can then be subjected to a Pd-mediated coupling with 2 equivalents of G13 to provide the compounds of formula G15. A compound of formula G15 can then be converted to the compounds of formula G17 via reaction with an aldehyde of formula G16 in the presence of an acid. Compounds of formula G17 can then be oxidized to provide the tetracyclic compounds of formula G18. Distereoisomers of G18 can be reparated by SFC using chiral columns.

In some of the Compounds of Formula (I) contemplated in Schemes 1-4, amino acids (such as, but not limited to proline, 4-(R)-fluoroproline, 4-(S)-fluoroproline, 4,4-difluoroproline, 4,4-dimethylsilylproline, aza-bicyclo[2.2.1]heptane carboxylic acid, aza-bicyclo[2.2.2]octane carboxylic acid, (S)-2-piperidine carboxylic acid, valine, alanine, norvaline, etc. . . . ) are incorporated as part of the structures. Methods have been described in the organic chemistry literature as well as in Banchard US 2009/0068140 (Published Mar. 9 2009) for the preparation of such amino acid-derived intermediates.

One skilled in the art of organic synthesis will recognize that the synthesis of fused tetracyclic cores contained in Compounds of Formula (I) may require protection of certain functional groups (i.e., derivatization for the purpose of chemical compatibility with a particular reaction condition). Suitable protecting groups for the various functional groups of these Compounds and methods for their installation and removal are well known in the art of organic chemistry. A summary of many of these methods can be found in Greene et al., Protective Groups in Organic Synthesis, Wiley-Interscience, New York, (1999).

One skilled in the art of organic synthesis will also recognize that one route for the synthesis of the fused tetracyclic cores of the Compounds of Formula (I) may be more desirable depending on the choice of appendage substituents. Additionally, one skilled in the art will recognize that in some cases the order of reactions may differ from that presented herein to avoid functional group incompatibilities and thus adjust the synthetic route accordingly.

One skilled in the art of organic synthesis will recognize that the synthesis of certain fused tetracyclic cores of the Compounds of Formula (I) require the construction of an amide bond. Methods useful for making such amide bonds, include but are not limited to, the use of a reactive carboxy derivative (e.g., an acid halide, or ester at elevated temperatures) or the use of an acid with a coupling reagent (e.g., HOBt, EDCI, DCC, HATU, PyBrop) with an amine.

The preparation of multicyclic intermediates useful for making the fused tetracyclic ring systems of the Compounds of Formula (I) have been described in the literature and in compendia such as “Comprehensive Heterocyclic Chemistry” editions I, II and III, published by Elsevier and edited by A. R. Katritzky & R. JK Taylor. Manipulation of the required substitution patterns have also been described in the available chemical literature as summarized in compendia such as “Comprehensive Organic Chemistry” published by Elsevier and edited by DH R. Barton and W. D. Ollis; “Comprehensive Organic Functional Group Transformations” edited by edited by A. R. Katritzky & R. JK Taylor and “Comprehensive Organic Transformation” published by Wily-CVH and edited by R. C. Larock.

The Compounds Formula (I) may contain one or more silicon atoms. The Compounds contemplated in this invention in general can be prepared using the carba-analog methodology unless otherwise noted. A recent review of the synthesis of silicon containing Compounds can be found in “Silicon Chemistry: from Atom to Extended Systems”, Ed P. Jutzi & U. Schubet; ISBN 978-3-527-30647-3. Preparation of silyl containing amino acids has been described. See Bolm et al., Angew. Chem. Int Ed., 39:2289 (2000). Descriptions of improved cellular update (Giralt, J. Am. Chem. Soc., 128:8479 (2006)) and reduced metabolic processing of silyl containing Compounds have been described (Johansson et al., Drug Metabolism & Disposition, 38:73 (2009)).

The starting materials used and the intermediates prepared using the methods set forth in Schemes 1-5 may be isolated and purified if desired using conventional techniques, including but not limited to filtration, distillation, crystallization, chromatography and alike. Such materials can be characterized using conventional means, including physical constants and spectral data.

One skilled in the art will be aware of standard formulation techniques as set forth in the open literature as well as in textbooks such as Zheng, “Formulation and Analaytical Development for Low-dose Oral Drug Products”, Wiley, 2009, ISBN.

### EXAMPLES

### Example 1

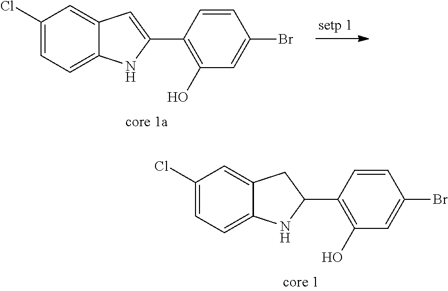 [SMILES: Oc1cc(Br)ccc1-c1cc2cc(Cl)ccc2[nH]1 ; Oc1cc(Br)ccc1C1Cc2cc(Cl)ccc2N1]

Compound core 1a was prepared as described in Example 19 of International Publication No. 2012/040923 A1. A solution of compound core 1a (5 g, 15.5 mmol) and Sn (9.2 g, 77.5 mmol) in conc.HCl/EtOH (50 mL) (conc.HCl:EtOH=1:1) was allowed to stir at 80° C. for 45 minutes. The mixture was filtered and the solid was dissolves in ethyl acetate, washed with NaHCO₃ aqueous, KF aqueous, brine, dried over Na₂SO₄, concentrated in vacuo. The solid was dissolved in 10 mL EtOAc, added 20 mL Petroleum Ether and stirred at 25° C. for 30 minutes. The mixture was filtered and the collected solid was washed with petroleum ether:EtOAc (2:1) to provide core 1 (2.5 g, 50% yield). ¹H NMR (CDCl₃) δ: 10.03 (br. s., 1H), 7.19 (d, J=8.2 Hz, 1H), 6.87-7.00 (m, 4H), 6.51 (d, J=8.2 Hz, 1H), 6.11 (br. s., 1H), 4.97 (t, J=8.2 Hz, 1H), 3.39 (dd, J=16.4, 9.4 Hz, 1H), 2.59 (dd, J=16.0, 7.8 Hz, 1H).

### Example 2

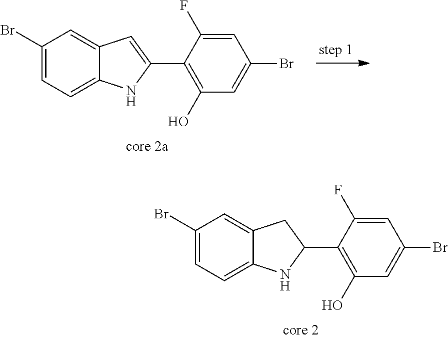 [SMILES: Oc1cc(Br)cc(F)c1-c1cc2cc(Br)ccc2[nH]1 ; Oc1cc(Br)cc(F)c1C1Cc2cc(Br)ccc2N1]

Compound core 2a was prepared as described in International Publication No. WO 2012/040923. Zn (80.0 g, 1.23 mol) was added to a solution of core 2a (40.0 g, 0.104 mol) in TFA (400 mL) at 76° C. The mixture was allowed to stir for 17 hours, then was cooled to room temperature and concentrated in vacuo. The residue obtained was washed with water (300 mL) and extracted with ethyl acetate (500 mL), washed with brine and dried over anhydrous sodium sulfate. After concentration in vacuo, the crude product was purified using SiO₂ chromatography (Hexane/EtOAc 10/1-5/1) to provide core 2 (18.0 g, 44.8% yield). LC/MS: Anal. Calcd. For [M+H]⁺ C14H10Br2FNO; 387.91. found 388.0.

### Example 3

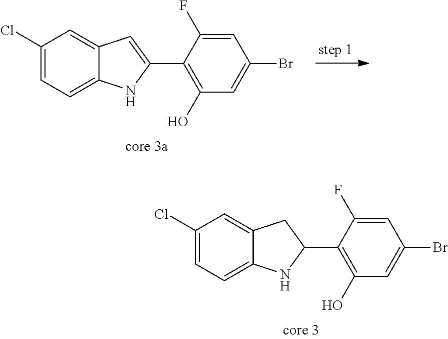 [SMILES: Oc1cc(Br)cc(F)c1-c1cc2cc(Cl)ccc2[nH]1 ; Oc1cc(Br)cc(F)c1C1Cc2cc(Cl)ccc2N1]

Compound core 3a was prepared as described in International Publication No. 2012/040923. To a mixture of core 3a (10 g, 0.029 mol), Zn (20 g, 0.31 mol) in TFA (120 mL) was added and the reaction was allowed to stir at 70° C. under N₂ atmosphere for about 15 hours. After cooling down, the mixture was filtered and concentrated in vacuo, extracted with EtOAc. Then NaHCO₃ was slowly added until the solution was at pH 8. The mixture was filtered and concentrated in vacuo. The residue obtained was purified using SiO₂ chromatography (Hexane/EtOAc 10:1-5:1) to provide core 3 (5 g, 50% yield). LC/MS: Anal. Calcd. For [M+H]⁺ C14H10BrClFNO; 343.59. found 343.9.

### Example 4

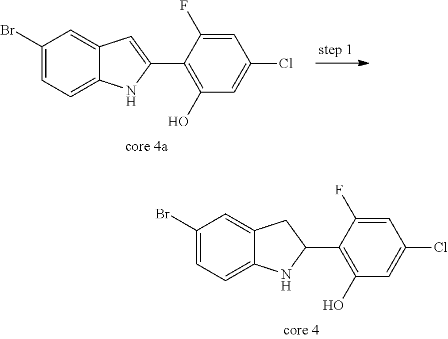 [SMILES: Oc1cc(Cl)cc(F)c1-c1cc2cc(Br)ccc2[nH]1 ; Oc1cc(Cl)cc(F)c1C1Cc2cc(Br)ccc2N1]

Compound core 4a was prepared as described in Example 19 of International Publication No. 2012/040923 A1. To a 500 mL flask was added compound core 4a (30 g, 88.06 mmol), Zinc (60 g, 923 mmol), and TFA (300 mL). The solution was allowed to stir at 75° C. for 24 hours. After cooling down, EtOAc (800 mL) and water (450 mL) was added. The organic layer was separated and washed with water two more time, Saturated NaHCO₃ twice, brine and dried over anhydrous Na₂SO₄. The solution was filtered and concentrated in vacuo. Crude Crude product was purified using SiO₂ chromatography (Hexane/EtOAc 0% to 30%) obtained compound core 4 (16 g, 53.3% yield). LC/MS: Anal. Calcd. For [M+H]⁺ C14H10BrClFNO; 343.59. found 343.9.

### Example 5

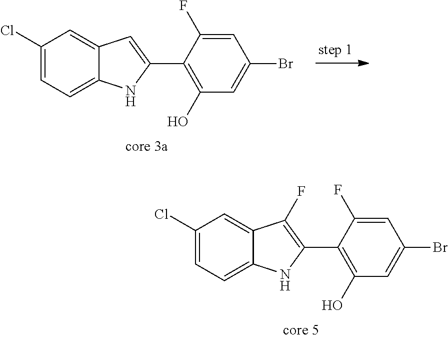 [SMILES: Oc1cc(Br)cc(F)c1-c1cc2cc(Cl)ccc2[nH]1 ; Oc1cc(Br)cc(F)c1-c1[nH]c2ccc(Cl)cc2c1F]

Compound core 3a was prepared as described in Example 19 of International Publication No. 2012/040923 A1. To a solution of core 3a (50 g, 0.147 mol) in 200 mL of MeCN and DMSO (V₁:V₂=1:1) at 0° C. in an ice bath. Selectfluor® (41.4 g, 0.117 mol) was added into the solution in portions and the resulting mixture was allowed to stir at 0° C. for 30 min. LC-MS was complete detected the reaction. The mixture was washed with water and extracted with DCM, dried over anhydrous Na₂SO₄, removed the DCM under reduced pressure and gained the crude product. Then the crude product was transferred to Pre-HPLC separation team to do purification and give core 5 as a black-red solid (24 g, yield 45%). LC/MS: Anal. Calcd. For [M+H]⁺ C14H7BrClF2NO; 357.94. found 358.1.

### Example 6

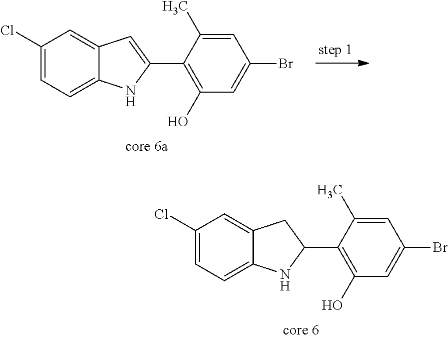 [SMILES: Cc1cc(Br)cc(O)c1-c1cc2cc(Cl)ccc2[nH]1 ; Cc1cc(Br)cc(O)c1C1Cc2cc(Cl)ccc2N1]

Compound core 6a was prepared as described in Example 19 of International Publication No. 2012/040923 A1. To a 100 mL flask was added core 6a (4 g, 11.88 mmol), zinc (7.77 g, 119 mmol), and TFA (59.4 mL). The solution was allowed to stir at 65° C. for 16 hours. After cooling down, EtOAc (200 mL) and water (150 mL) was added. The organic layer was separated and washed with water two more time, Saturated NaHCO₃ twice, brine and dried over anhydrous Na₂SO₄. The solution was filtered and concentrated in vacuo. Product was purified using SiO₂ chromatography (120 g, Hexane/EtOAc 0% to 30%) to provide core 6 (2.8 g, 69.6%).

### Example 7

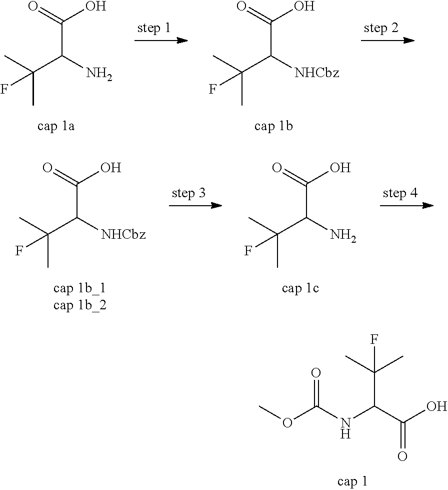

### Step 1

Cap 1a was prepared as described in J. Fluorine. Chem. 1984, 24, 137-151. To a solution of cap 1a (9 g, 66.7 mmol) in MeOH (100 mL) at 0° C. was added Et₃N (14.8 g, 146.7 mmol) and CbzCl (11.3 g, 66.7 mmol). The solution was allowed to stir at 25° C. for about 15 hours. After completion of the reaction, the reaction solution was adjusted pH=3 with HCl (1N in water), extracted with EtOAc, the organic phase was separated, washed with brine, dried over Na₂SO₄ and concentrated in vacuo to provide cap 1b (15 g, yield 89%). LC/MS: Anal. Calcd. For [M+H]⁺ C13H16FNO4; 270.11. found 270.1.

### Step 2

Cap 1b was separated by Supercritical Fluid Chromatogarphy (SFC) using the following conditions to provide the isomeric compounds cap 1b_1 (7 g, yield 40%) and cap 1b_2 (7 g, yield 40%). LC/MS: Anal. Calcd. For [M+H]⁺ C13H16FNO4; 270.11. found 270.12.

Column: chiralpak AD-3 150×4.6 mm I.D.

Mobile phase: iso-propanol (0.05% DEA) in CO₂ from 5% to 40%

Flow rate: 2.5 mL/min

Wavelength: 220 nm

### Step 3

To a solution of cap 1b_1 (3.5 g, 13 mmol) in MeOH (50 mL) was added Pd/C (10%, 0.1 g) carefully. Then the reaction mixture was allowed to stir at 25° C. under H₂ (15 psi) for 6 hours. After completion of the reaction, Pd/C was filtered and the solvent was removed in vacuo. The desired compound cap 1c was obtained as a white solid (1.1 g, 67% yield).

### Step 4

To a solution of cap 1c (0.87 g, 6.5 mmol) in DCM (20 mL) was added Et₃N (1.44 g, 14.3 mmol) at 0° C. After stirring for 10 minutes, methyl chloroformate (0.66 mg, 7.1 mmol) was added in dropwise at 0° C.; then the reaction solution was allowed to stir at 25° C. for 3 hours. After completion of the reaction, the reaction solution was adjusted pH with HCl (1N in water) to 3, extracted with ethyl acetate; the organic phase was separated, washed with brine, dried over Na₂SO₄ and concentrated in vacuo. The residue obtained was purified using Pre-HPLC to provide the desired compound cap 1 (1.2 g, 67% yield). LC/MS: Anal. Calcd. For [M+H]⁺ C5H8FO2; 120.05. found 120.25.

### Example 8

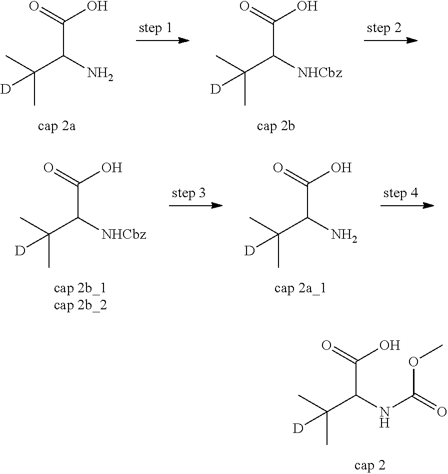

### Step 1

To a solution of cap2a (0.5 g, 4.2 mmol) in MeOH (50 mL) at 0° C. was added Et₃N (0.929 g, 9.2 mmol) and CbzCl (0.785 g, 4.62 mmol). The solution was allowed to stir at 25° C. for about 15 hours. After completion of the reaction, the reaction solution was adjusted to pH 3 with HCl (1N in water), extracted with EtOAc, the organic phase was separated, washed with brine, dried over Na₂SO₄ and concentrated in vacuo to provide compound cap 2b (0.8 g, yield 82%). LC/MS: Anal. Calcd. For [M+H]⁺ C13H16DNO4; 253.11. found 253.12.

### Step 2

Cap 2b was separated by SFC using the following conditions to provide two chiral compounds cap 2b_1 and cap 2b_2.

Column: Chiralpak IC

Mobile phase: iso-propanol (0.05% DEA) in CO₂ from 5% to 40%

Flow rate: 2.5 mL/min

Wavelength: 220 nm

Cap 2b_1 (0.2 g, yield 50%). LC/MS: Anal. Calcd. For [M+H]⁺ C13H16DNO4: 253.11. found 253.1.

### Step 3

To a solution of cap 2b_1 (0.2 g, 0.79 mmol) in MeOH (20 mL) was added Pd on carbon (10%, 0.1 g) carefully. Then the reaction mixture was allowed to stir at 25° C. under H₂ (15 psi) for 6 hours. After completion of the reaction, Pd/C was filtered and the solvent was removed in vacuo. Cap 2a_1 was obtained as a white solid (0.1 g, yield 98%).

### Step 4

To a solution of cap 2a_1 (0.1 g, 0.8 mmol) in DCM (10 mL) was added Et₃N (0.171 g, 1.7 mmol) at 0° C. After stirring for 10 minutes, methyl chloroformate (82 mg, 0.88 mmol) was added dropwise at 0° C.; then the reaction solution was allowed to stir at 25° C. for 3 hours. After completion of the reaction, the reaction solution was adjusted to pH 3 with HCl (1N in water), extracted with EtOAc; the organic phase was separated, washed with brine, dried over Na₂SO₄ and concentrated in vacuo. The residue obtained was purified using Pre-HPLC to provide cap 2 (0.08 g, yield 57%). LC/MS: Anal. Calcd. For [M+H]⁺ C7H12DNO4; 177.09. found 177.12.

### Example 9

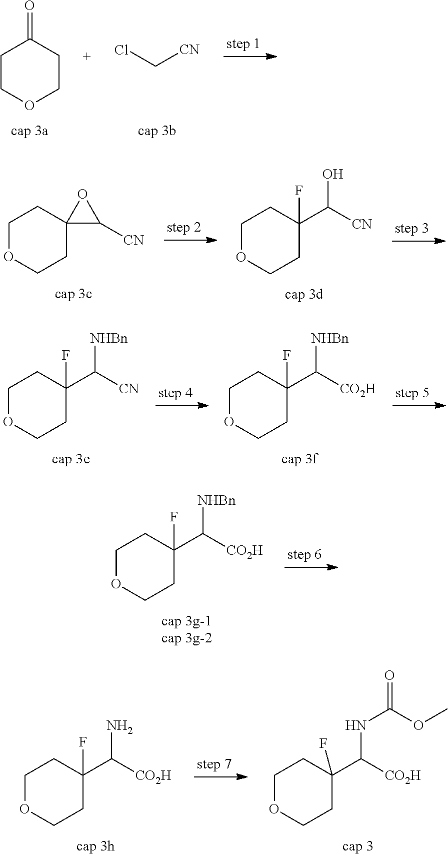

### Step 1

To a solution of cap 3a (20 g, 0.2 mol) in tBuOH (500 mL) was added cap 3b (15 g, 0.2 mol). Then a solution of KOtBu (22.4 g, 0.2 mol) in t-BuOH (500 mL) was added dropwise. The reaction solution was allowed to stir at 25° C. for 1 hour. The mixture was poured into water and extracted with EtOAc. The organic layers were washed with brine and dried over Na₂SO₄, then concentrated in vacuo to provide crude cap 3c (19 g, 71% yield).

### Step 2

To a solution of cap 3c (7 g, 50 mmol) in DCM (20 mL) was added HF.Py (20 mL). The reaction mixture was allowed to stir at 25° C. for 12 hours. The mixture was poured into ice and extracted with DCM. The organic layers were washed with brine and dried over Na₂SO₄, then concentrated in vacuo and purified using column chromatography (100-200 mesh, Petroleum Ether/EtOAc=5/1 to 2/1) to provide cap 3d (4 g, 50% yield). ¹H NMR (CDCl₃): δ 6.96 (d, J=6.8 Hz, 1H), 4.68 (dd, J=6.8 Hz, 16.8 Hz, 1H), 3.78 (dd, J=4.4 Hz, 12 Hz, 2H), 3.40-3.55 (m, 2H), 1.60-1.90 (m, 4H).

### Step 3

To a solution of cap 3d (1.59 g, 10 mmol) in MeOH (20 mL) was added benzyl amine (1.07 g, 10 mmol) and MgSO₄ (2.4 g, 20 mmol). The reaction mixture was allowed to stir at 25° C. for 12 hours. The solid was filtered and the filtrate was concentrated in vacuo. The crude product was purified using column chromatography (100-200 mesh, Petroleum Ether/EtOAc=3/1 to 1/1) to provide cap 3e (2 g, 80% yield). LC/MS: Anal. Calcd. For [M+H]⁺ C14H17FN2O; 249.13. found 249.1.

### Step 4

To HCl (6 N, 20 mL) was added cap 3e (2 g, 8 mmol). The reaction mixture was allowed to stir at reflux for 3 days. The solvent was removed in vacuo and residual was purified using Pre-HPLC to provide cap 3f (0.2 g, 10% yield). LC/MS: Anal. Calcd. For [M+H]⁺ C14H18FNO3; 268.13. found 268.2.

### Step 5

Compound cap 3f (0.18 g, 0.67 mmol) was separated by SFC under the following condition to provide cap 3g-1 and cap 3g-2.

Instrument: Thar SFC

Column: AS-H, 150×4.6 mm, 5 um

Mobile phase: A for CO₂ and B for EtOH (0.05% DEA)

Gradient: B 5% to 40 for A

Flow rate: 2.35 mL/min

Back pressure: 100 bar

Column temperature: 35° C.

Wavelength: 230 nm

Cap 3g-1 (60 mg, 67% yield). LC/MS: Anal. Calcd. For [M+H]⁺ C14H18FNO3: 268.13. found 268.2.

Cap 3g-2 (60 mg, 67% yield). LC/MS: Anal. Calcd. For [M+H]⁺ C14H18FNO3: 268.13. found 268.2.

### Step 6

To a solution of cap 3g-2 (53 mg, 0.05 mmol) in MeOH (5 mL) was added Pd/C (10%, 20 mg). The reaction mixture was allowed to stir at 40° C. under H₂ (50 Psi) for 12 hours. The solid was filtered and the filtrate was concentrated in vacuo to provide the crude product cap 3h (30 mg, 88% yield).

### Step 7

To a solution of cap 3h (38 mg, 0.22 mmol) in MeOH (5 mL) was added Et₃N (50 mg, 0.5 mmol), methyl carbonochloridate (28.5 mg, 0.3 mmol) dropwise at 0° C. The reaction was allowed to stir at 25° C. for 12 hours. The solvent was removed in vacuo and the residual was washed with water and extracted with EtOAc. The solvent was removed to provide cap 3 (22 mg, 48% yield). ¹H NMR (MeOD): δ 4.27 (d, J=19.6 Hz, 1H), 3.65 (s, 3H), 3.59-3.85 (m, 4H), 1.78-2.02 (m, 4H).

### Example 10

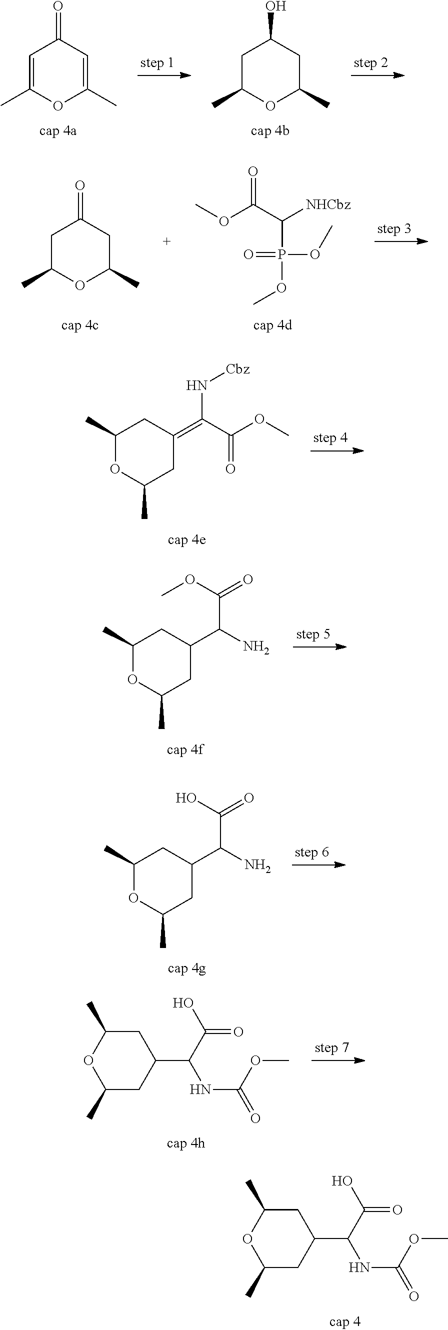

### Step 1

The solution of compound cap 4a (73 g, 0.59 mol) in ethanol was added Pd/C (10%, 4 g) and the reaction was allowed to stir at 35° C. under H₂ (50 Psi) for 17 hours. The reaction was filtered through CELITE and the volatiles were removed in vacuo to provide cap 4b (76 g, 99% yield). ¹H NMR: (CDCl₃) δ: 3.75 (s, 1H), 3.44-3.40 (m, 2H), 1.88 (d, J=16 Hz, 2H), 1.19 (d, J=8 Hz, 6H), 1.14-1.08 (m, 2H).

### Step 2

To a solution of cap 4b (74.7 g, 0.57 mol) in DCM (750 mL) was added a solution of NaHCO₃ (4.83 g, 57 mmol) and KBr (6.84 g, 57 mmol) in water (200 mL). Then TEMPO (0.9 g, 5.7 mmol) was added. The mixture was treated at 0° C. under vigorous stirring with NaClO aqueous (47.1 g, 0.63 mol, 5%˜7%) over 1 hour. Then the whole system was allowed to stir at 25° C. for 5 hours and the aqueous layer was extracted with DCM. The organic phase was washed with brine, dried over Na₂SO₄ and the solvent evaporated to provide compound cap 4c as pale yellow oil (72.2 g, 99% yield). ¹H NMR: (CDCl₃) δ: 3.75-3.70 (m, 2H), 2.33 (d, J=16 Hz, 2H), 2.19 (t, J=24 Hz, 2H), 1.31 (d, J=6 Hz, 6H).

### Step 3

To a solution of cap 4d (124 g, 0.38 mol) in dry DCM (160 mL) was added DBU (57.2 g, 0.038 mol) dropwise at 0° C. Then a solution of compound cap 4c (72.2 g, 0.56 mol) in dry DCM (160 mL) was added dropwise at 0° C. The reaction mixture was allowed to stir at 25° C. for 20 hours. After removal of the solvent, the residue obtained was purified using SiO₂ chromatography to provide cap 4e (90 g, 71% yield). ¹H NMR: (MeOD) δ: 7.35-7.31 (m, 5H), 5.10 (s, 2H), 3.68 (s, 3H), 3.48-3.44 (m, 3H), 2.63-2.60 (m, 1H), 1.82-1.68 (m, 2H), 1.25 (s, 6H).

### Step 4

To a solution of cap 4e (45 g, 0.135 mol) in MeOH (450 mL) was added Pd/C (10%, 3 g) carefully. Then the reaction mixture was allowed to stir at 25° C. under H₂ (35 psi) for 8 hours. After completion of the reaction, Pd/C was filtered and the solvent was removed in vacuo. Cap 4f was obtained as colorless oil (27.8 g, 100% yield). ¹H NMR: (MeOD) δ: 3.71 (s, 3H), 3.49-3.44 (m, 2H), 3.25 (d, J=6 Hz, 1H), 1.93-1.88 (m, 1H), 1.62 (d, J=4 Hz, 1H), 1.58 (d, J=4 Hz, 1H), 1.15 (d, J=4 Hz, 6H), 1.05-0.91 (m, 2H).

### Step 5

To a solution of cap 4f (27.8 g, 0.14 mol) in MeOH (300 mL) was added a solution of NaOH (11.05 g, 0.28 mol) in water (100 mL) and the reaction solution was refluxed for 35 hours. After completion of the reaction, the solvent was removed in vacuo and the crude cap 4g was used next step directly. LC/MS: Anal. Calcd. For [M+H]⁺ C9H17NFO3; 188.12. found: 188.1.

### Step 6

To a solution of cap 4g (26.2 g, 0.14 mol) in H₂O (260 mL) was added NaOH (2.8 g, 0.07 mol) at 0° C. After stirring for 10 min, methyl chloroformate (14.4 g, 0.15 mol) was added in dropwise at 0° C.; then the reaction solution was allowed to stir at 25° C. for 3 hours. After completion of the reaction, the reaction solution was adjusted pH=3 with HCl (1N), extracted with EtOAc, the organic phase was separated, washed with brine, dried over Na₂SO₄ and concentrated in vacuo. The residue obtained was purified using PRE-HPLC to provide the cap 4h (16 g, 53% yield). LC/MS: Anal. Calcd. For [M+H]⁺ C11H19NFO5; 246.13. found: 246.1.

### Step 7

The compound cap 4h (16 g) was separated by SFC to provide compounds cap 4 by the following method:

Instrument: Thar SFC

Column: AY-5, 150×4.6 mm, 5 um

Mobile phase: A for CO₂ and B for EtOH (0.05% DEA)

Gradient: B 5% to 40 for A

Flow rate: 2.5 mL/min

Back pressure: 100 bar

Column temperature: 35° C.

Wavelength: 230 nm

Compounds cap 4 (5.4 g, 34% yield). ¹H NMR (MeOD) δ: 4.01 (d, J=6 Hz, 1H), 3.62 (s, 3H), 3.46-3.43 (m, 2H), 2.12-2.07 (m, 1H), 1.61-1.52 (m, 2H), 1.13 (d, J=6 Hz, 6H), 1.03-0.94 (m, 2H).

### Example 11

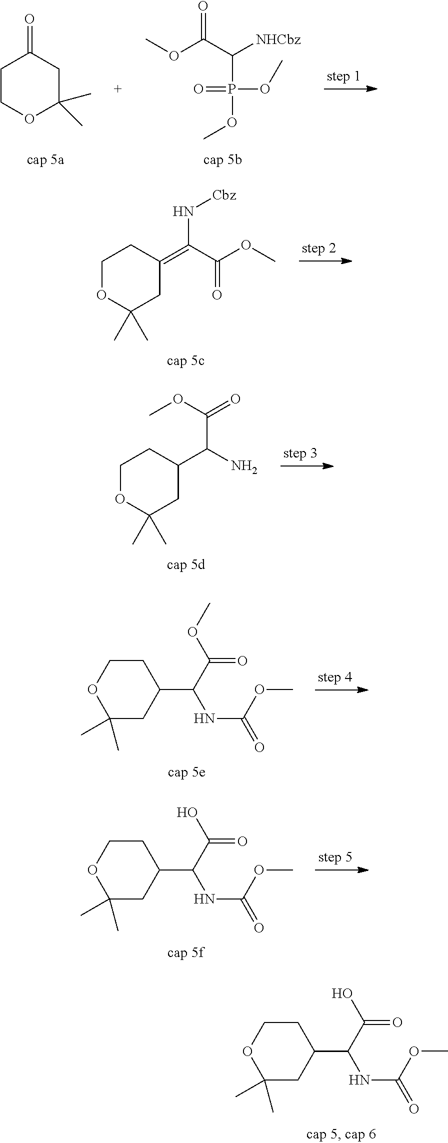

### Step 1

To a solution of cap 5b (1.163 g, 3.52 mmol) in dry DCM (20 mL) was added DBU (0.534 g, 3.52 mmol) dropwise at 0° C. Then a solution of cap 5a (1.8 g, 14.08 mmol) in dry DCM (20 mL) was added dropwise at 0° C. The reaction mixture was allowed to stir at 25° C. for 3 days. After removal of the solvent, the residue obtained was purified using SiO₂ chromatography (eluting with petroleum ether/ethyl acetate=5:1 to 3:1) to provide cap 5c as a white solid (0.15 g, 13% yield). ¹H NMR (CDCl₃): δ 7.30-7.35 (m, 5H), 5.11 (s, 2H), 3.82-3.88 (m, 3H), 3.09-3.16 (m, 2H), 1.84 (s, 2H), 1.48 (s, 2H).

### Step 2

To a solution of cap 5c (3 g, 9.01 mmol) in MeOH (100 mL) was added Pd/C (0.6 g) carefully under N₂. Then the reaction mixture was allowed to stir at 25° C. under H₂ (45 psi) for 3 hours. After completion of the reaction, Pd/C was filtered and the solvent was removed in vacuo. Cap 5d was obtained as colorless oil (1.8 g, 99% yield). LC/MS: Anal. Calcd. For [M+H]⁺ C10H19NFO3; 202.14. found: 202.1.

### Step 3

To a solution of compound cap 5d (1.7 g, 8.46 mmol) in dry DCM (40 mL) was added DIPEA (1.65 g, 12.69 mmol) and methyl chloroformate (0.964 g, 10.15 mmol) dropwise at 0° C. The reaction solution was allowed to stir at 25° C. for 2 hours. After completion of the reaction, water and DCM was added. The organic phase was separated, washed with brine, dried over Na₂SO₄ and concentrated in vacuo. Cap 5e was obtained as colorless oil (1.9 g, 87% yield). LC/MS: Anal. Calcd. For [M+H]⁺ C12H21NFO5; 260.14. found: 260.2.

### Step 4

To a solution of cap 5e (1.9 g, 7.34 mmol) in THF/H₂O (20 mL/4 mL) was added LiOH (0.264 g, 11.00 mL) at 25° C. for 4 hours. After completion of the reaction, 1 N HCl was added to adjust the pH value to 6. Then the organics were extracted with DCM. The organic phase was washed with brine, dried over Na₂SO₄. After removal of the solvent, the crude was purified using Pre-HPLC to provide compound cap 5f as a white solid (1 g, 56% yield). LC/MS: Anal. Calcd. For [M+H]⁺ C11H19NFO5; 246.13. found: 246.1.

### Step 5

Compound cap 5f (1 g) was separated by SFC under the following condition to provide Cap 5 and Cap 6.

Instrument: Thar SFC

Column: AS-H, 150×4.6 mm, 5 um

Mobile phase: A for CO₂ and B for EtOH (0.05% DEA)

Gradient: B 5% to 40 for A

Flow rate: 2.5 mL/min

Back pressure: 100 bar

Column temperature: 35° C.

Wavelength: 230 nm

Cap 5 (170 mg, 17% yield). LC/MS: Anal. Calcd. For [M+H]⁺ C11H19NFO5: 245.13. found: 246.1.

Cap 6 (230 mg, 23% yield). LC/MS: Anal. Calcd. For [M+H]⁺ C11H19NFO5: 245.13. found: 246.1.

### Example 12

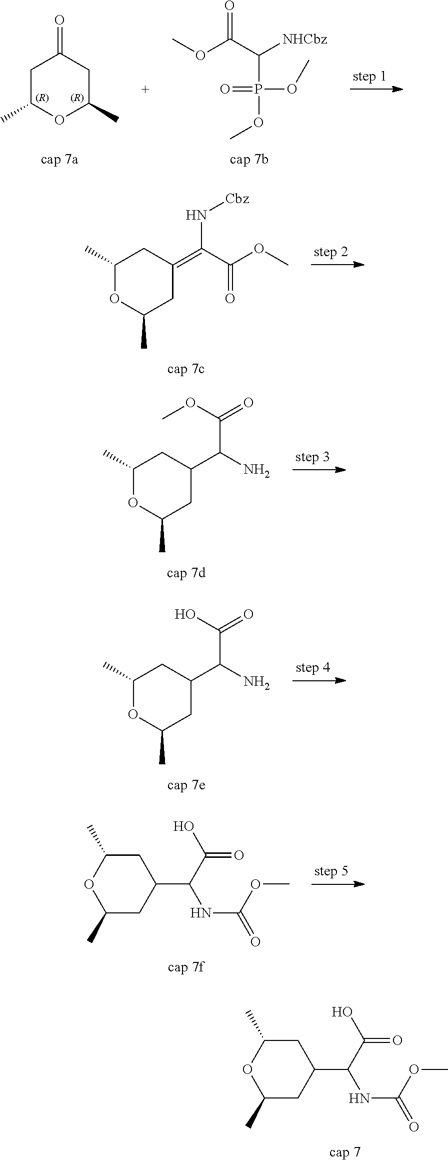

### Step 1

To a solution of cap 7b (3.3 g, 10 mmol) in dry DCM (50 mL) was added DBU (1.52 g, 10 mmol) dropwise at 0° C. Then a solution of compound cap 7a (1.9 g, 14.7 mmol) in dry DCM (50 mL) was added dropwise at 0° C. The reaction mixture was allowed to stir at 25° C. for 20 hours. After removal of the solvent, the residue obtained was purified using SiO₂ chromatography to provide compound cap 7c (3.6 g, 35% yield). LC/MS: Anal. Calcd. For [M+H]⁺ C18H23NO5; 334.16. found 334.52.

### Step 2

To a solution of compound cap 7c (3.6 g, 10.8 mmol) in MeOH (50 mL) was added Pd/C (10%, 0.2 g) carefully. Then the reaction mixture was allowed to stir at 25° C. under H₂ (35 psi) for 8 hours. After completion of the reaction, Pd/C was filtered and the solvent was removed in vacuo. The desired compound cap 7d was obtained as colorless oil (2 g, 99% yield). LC/MS: Anal. Calcd. For [M+H]⁺ C10H19NO3; 202.14. found 202.24.

### Step 3

To a solution of cap 7d (2 g, 10 mmol) in MeOH (21 mL) was added a solution of LiOH.H₂O (840 mg, 20 mmol) in water (7 mL) and the reaction solution was allowed to stir for 8 hours. After completion of the reaction, the solvent was removed in vacuo and the crude compound cap 7e was used in the next step directly.

### Step 4

To a solution of cap 7e in H₂O was added LiOH.H₂O (0.42 g, 10 mmol) and Na₂CO₃ (3.2 g, 30 mmol) at 0° C. After stirring for 10 minutes, methyl chloroformate (1.1 g, 12 mmol) was added in dropwise at 0° C.; then the reaction solution was allowed to stir at 25° C. for 3 hours. After completion of the reaction, the reaction solution was adjusted pH=3 with HCl (1N), extracted with ethyl acetate, the organic phase was separated, washed with brine, dried over Na₂SO₄ and concentrated in vacuo. The residue obtained was purified using Pre-HPLC to provide cap 7f (1.4 g, 52% yield). LC/MS: Anal. Calcd. For [M+H]⁺ C11H19NO5; 246.13. found 246.54.

### Step 5

Compound cap 7f (1.4 g) was separated by SFC using the following conditions to provide cap 7.

Column: Chiralpak AS-H 250×4.6 mm I.D.

Mobile phase: ethanol (0.05% DEA) in CO₂ from 5% to 40%

Flow rate: 2.35 mL/min

Wavelength: 220 nm

Cap 7 (0.5 g, 35% yield). LC/MS: Anal. Calcd. For [M+H]⁺ C11H19NO5; 246.13. found 246.53.

### Example 13

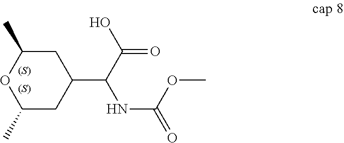

Cap 8 was made according to the same method described in Example 7.

### Example 14

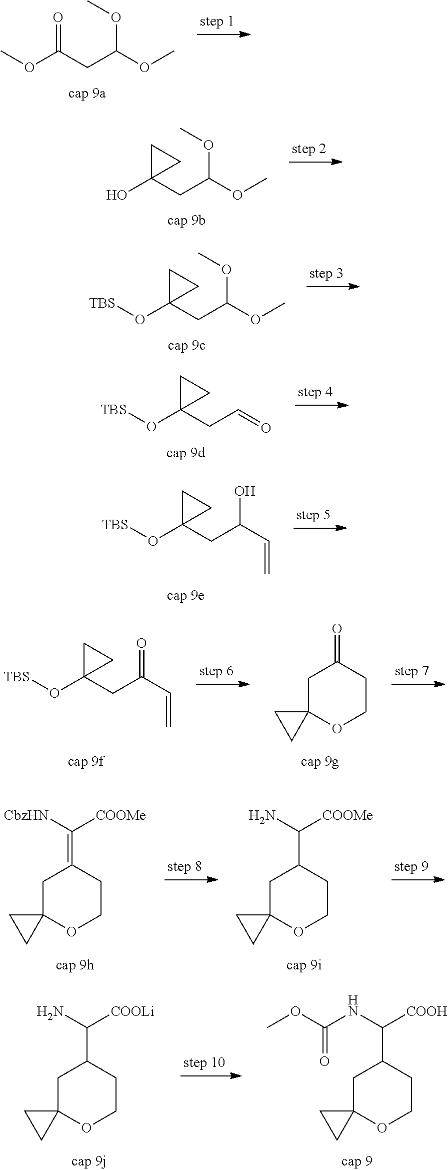

### Step 1

To a solution of cap 9a (100 g, 0.52 mol) in THF/Et₂O (600/600 mL) was added Titanium isopropoxide (29 g, 0.1 mol) at 0° C. with stirring. After stirring for 10 minutes, ethylmagnesium bromide (434 mL, 3M in Et₂O, 1.3 mol) was added dropwise at 10˜15° C. After addition complete, the solution was allowed to stir at 25° C. for 30 minutes. The reaction was quenched with 6 mL of water. The mixture was filtered. The filtrate was concentrated in vacuo. The residue obtained was purified using column chromatography (Petroleum Ether/EtOAc=20:1-4:1) to provide cap 9b (80 g, 89% yield).

### Steps 2&3

To a cap 9b (80 g, 0.46 mmol) in dichloromethane (600 mL) was added 2, 6-lutidine (246 g, 2.3 mol), TBSOTf (121 g, 0.46 mmol) at 10° C. After stirring at 10° C. for 30 minutes, to the reaction mixture was added TMSOTf (153 g, 0.7 mol). After stirring for 1 hour, the reaction mixture was poured into water, extracted with ethyl acetate (3 times). The combined organic phases were dried. After concentration, the residue obtained was purified using column (Petroleum Ether/EtOAc=100:1-20:1) to provide cap 9d (40 g, 41% yield).

### Step 4

To a solution of cap 9d (40 g, 180 mmol) in THF (400 mL) was added villylmagnesium bromide (175 mL, 1.6 M in THF, 280 mmol) at −78° C. with stirring. After stirring for 30 minutes, the reaction mixture was quenched with aqueous NH₄Cl, and extracted with ethyl acetate (3 times). The combined organic phases were dried. After concentration, the residue obtained was purified using column (Petroleum Ether/EtOAc=10:1-5:1) to provide cap 9e (20 g, 41% yield).

### Step 5

To a solution of cap 9e (20 g, 82 mmol) in acetonitrile (200 mL) was added IBX (46 g, 165 mmol) at 25° C. Then the mixture was allowed to stir at 80° C. for 2 hours. The mixture was filtered. The filtrate was concentrated in vacuo to the crude product. It was purified using column chromatography give cap 9f (10 g, 50% yield).

### Step 6

To a solution of cap 9f (4.8 g, 20 mmol) in DCM (20 mL) was added Amberlyst 15 (2 g). The mixture was refluxed for 4 hours. The mixture was filtered. The filtrate was concentrated in vacuo to provide the crude cap 9g (2.5 g, 100% yield).

### Step 7

To a solution of cap 9g (2.5 g, 20 mmol) in DCM (10 mL) was added DBU (6 g, 40 mmol) and benzyloxycarbonyl-α-phosphonoglycine trimethyl ester (6.6 g, 20 mmol). The mixture was allowed to stir at 25° C. for 4 hours. The mixture was poured into water, and extracted with ethyl acetate (3 times). The combined organic phases were dried. After concentration, the residue obtained was purified using column (Petroleum Ether/EtOAc=40:1-10:1) to provide cap 9h (0.66 g, 10% yield).

### Step 8

To a solution of cap 9h (1.65 g, 5 mmol) in methanol (50 mL) was added Pd/C (0.4 g) at 25° C. Then the mixture was allowed to stir at 45° C. under 50 psi H₂ for about 15 hours. After the mixture was filtered, the filtration was concentrated in vacuo to provide cap 9i (1 g, 100% yield).

### Step 9&10

To a solution of cap 9i (1 g, 5 mmol) in methanol (15 mL) was added LiOH (1 g, 25 mmol). The mixture was allowed to stir at 25° C. for 1 hour. Then to the mixture was added MocCl (940 mg, 10 mmol). The mixture was allowed to stir at 25° C. for about 15 hours the mixture was poured into water, neutralized by aqueous NaHCO₃ and extracted. The organic layers were dried over Na₂SO₄ and concentrated in vacuo to provide the cap 9 (200 mg, 16.7% yield).

### Example 15

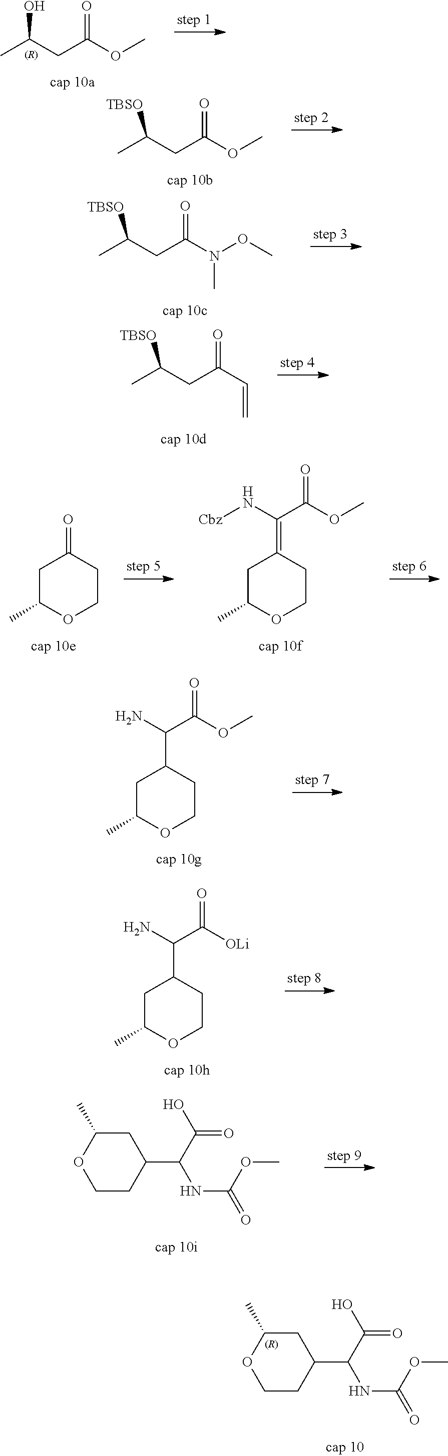

### Step 1

Compound cap 10a (174 g, 1475 mmol) and imidazole (109 g, 1622 mmol) was dissolved in ethyl acetate (1 L). After cooling to 0° C., tertbutyl chlorodimethylsilane (242 g, 1662 mmol) in ethyl acetate (300 mL) was added dropwise and kept the internal temperature below 10° C. After stirring the thick suspension at 25° C. for 16 hours, water (800 mL) was added, the layers were separated and the aqueous layer was extracted with ethyl acetate (450 mL). The combined organic layers were washed with brine (450 mL), dried over Na₂SO₄ and the solvent evaporated under reduced pressure to provide compound cap 10b as colorless oil (322 g, 95% yield). ¹H NMR (CDCl₃) δ: 4.28-4.13 (m, 1H), 3.60 (s, 3H), 2.47-2.36 (m, 1H), 2.35-2.28 (m, 1H), 1.13 (d, J=6.0 Hz, 3H), 0.80 (s, 9H), 0.00 (d, J=9.5 Hz, 6H). ° C.

### Step 2

Compound cap 10b (322 g, 1.4 mol) was dissolved in THF (250 mL) and N, O-dimethylhydroxylamine hydrochloride salt (155 g, 1.6 mol) was added. After cooling the slurry to −20° C., a solution of isopropylmagnesium chloride in THF (2.0 M, 1.6 L, 3.2 mol) was added dropwise over 1 hour, maintaining the reaction temperature at below 0° C. After keeping at −20° C. for further 2 hours, the TLC show the reaction was completely done, the reaction was quenched with a saturated aqueous of ammonium chloride (2 L) and diluted with ethyl acetate (500 mL). The layers were separated, the aqueous layer was extracted with ethyl acetate (400 mL), the combined organic layers were washed with brine (500 mL) and dried (Na₂SO₄), and the solvent was evaporated under reduced pressure to leave crude product which purified using SiO₂ chromatography elution with petroleum ether/ethyl acetate=20:1 to provide cap 10c as colorless oil (260 g, 72% yield). ¹H NMR (CDCl3) δ: 4.39-4.24 (m, 1H), 3.66 (s, 3H), 3.13 (s, 3H), 2.73 (m, 1H), 2.31 (dd, J=4, 12 Hz, 1H), 1.17 (d, J=8 Hz, 3H), 0.82 (s, 9H), 0.01 (d, J=12 Hz, 6H)

### Step 3

To a solution of vinylmagnesium bromide in THF (1.0 M, 1.6 L, 1.6 mol) at 0° C. under nitrogen atmosphere cap 10c (260 g, 1 mol) was added dropwise over 30 minutes, maintaining the reaction temperature below 5° C. After keeping for a further 1 hour at between 0 to 5° C., the solution was quenched into a stirred mixture of saturated aqueous citric acid (1 L) and ammonium chloride (1000 mL) (with some ice keep the temperature below 5° C.). The layers were separated, the aqueous layer was extracted with ethyl acetate (1 L), the combined organic layers were washed with (500 mL) sodium carbonate aqueous and brine, dried over Na₂SO₄, and the solvent was evaporated under reduced pressure. The residue obtained was purified using SiO₂ chromatography, eluting with a 1:100 mixture of ethyl acetate and petroleum ether to provide cap 10d as colorless oil (180 g, 79% yield). ¹H NMR (CDCl₃) 6.36 (dd, J₁=17.7, J₂=10.5 Hz, 1H), 6.22 (dd, J₁=17.7, J₂=1.3 Hz, 1H), 5.85 (dd, J₁=10.5, J₂=1.3 Hz, 1H), 4.37-4.33 (m, 1H), 2.85 (dd, J₁=14.9, J₂=7.2 Hz, 1H), 2.54 (dd, J₁=14.9, J₂=5.4 Hz, 1H), 1.20 (d, J=6.2 Hz, 3H), 0.86 (s, 9H), 0.06 (s, 3H), 0.02 (s, 3H).

### Step 4

To a solution of compound cap 10d (180 g, 789 mmol) in chloroform (500 mL) at 25° C. Amberlyst 15 resin (100 g) was added. The mixture was allowed to stir at 25° C. for about 15 hours and TLC shown the starting material was completely consumed. The mixture was filtered and the filtrate was crude cap 10e which was directly used for the next step without further work up.

### Step 5

To a solution of crude cap 10e (789 mmol) and methyl 2-(((benzyloxy) carbonyl) amino)-2-(dimethoxyphosphoryl) acetate (277 g, 789 mmol) in chloroform (800 mL) was added DBU (120 g, 789 mmol) dropwise at 0° C. Then the reaction mixture was allowed to stir at 25° C. for 5 hours. The resulting mixture was diluted with saturate ammonium chloride aqueous. The organic layer was separated and washed by brine, dried over Na₂SO₄, and the solvent was evaporated under reduced pressure. The residue obtained was purified using SiO₂ chromatography, eluting with a 1:1:1 mixture of ethyl acetate and petroleum ether and dichloromethane to provide cap 10f as a white solid (175 g, 70% yield).

### Step 6

To a solution of cap 10f (27 g, 84.6 mmol) in MeOH (250 mL) was added Pd/C (10%, 3 g) carefully. Then the reaction mixture was allowed to stir at 25° C. under H₂ (35 psi) for 8 hours. After completion of the reaction, Pd/C was filtered and the solvent was removed in vacuo. Compound cap 10g was obtained as colorless oil (13 g, 82% yield).

### Step 7

To a solution of cap 10g (13 g, 69.5 mmol) in MeOH (40 mL) was added a solution of LiOH (4.38 g, 104 mmol) in water (140 mL). The mixture was allowed to stir at 25° C. for about 15 hours. After completion of the reaction, the solvent was removed in vacuo and the residue obtained was crude cap 10h which was used for next step directly.

### Step 8

To a solution of the crude cap 10h (11.9 g, 69 mmol) in H₂O (200 mL) was added LiOH (4.2 g, 69 mmol) at 0° C. After stirring for 10 minutes, methyl chloroformate (9.4 g, 100 mmol) was added in dropwise at 0° C.; then the reaction solution was allowed to stir at 25° C. for 3 hours. After completion of the reaction, the reaction solution was extracted with METB (200 mL×2) and the aqueous layer was adjusted pH with HCl (1N) to 3, extracted with ethyl acetate, the organic phase was separated, washed with brine, dried over Na₂SO₄ and concentrated in vacuo to provide cap 10i (10 g, 63% yield).

### Step 9

Compound cap 10i (10 g) was separated by SFC by the following conditions to provide cap 10.

Column: AS-H 250×4.0 mm

Solvent: 35% MeOH (0.05% DEA)/CO₂

Flow rate: 2.35 mL/min

Wavelength: 220 nm

Compound cap 10 (2 g, 20% yield). LC/MS: Anal. Calcd. For [M+H]⁺ C10H17NO5: 232.11. found 232.1.

### Example 16

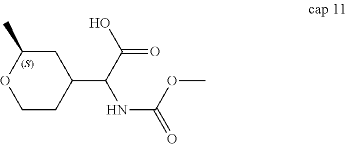

Cap 11 was prepared as described in International Publication No. WO2011/075439.

### Example 17

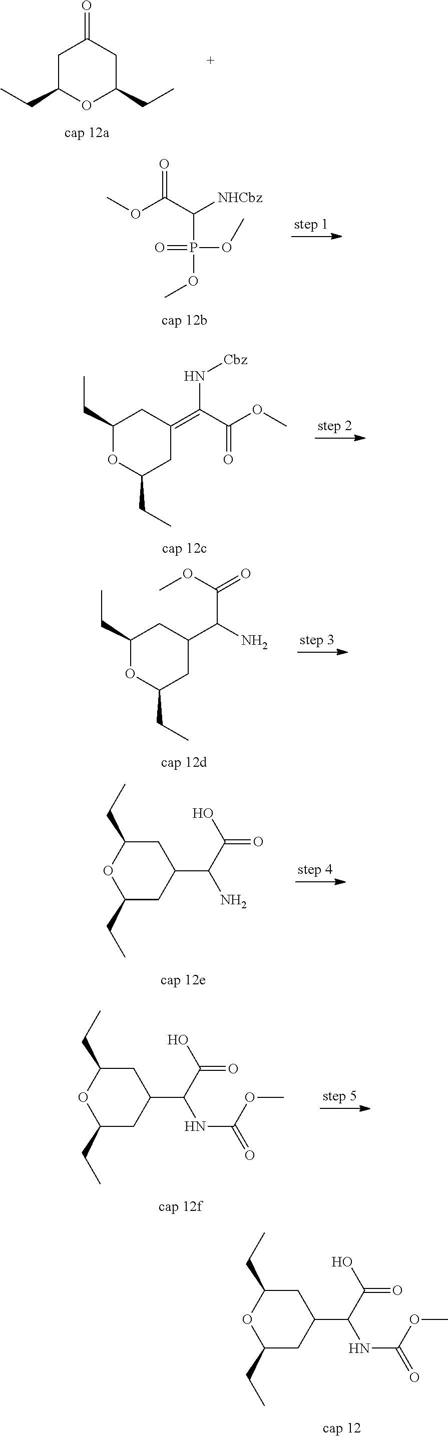

### Step 1

Cap 12a was prepared according to the literature Monatsh. Chem., 2005, 136, 1197-1203. To a solution of cap 12b (1.41 g, 4.27 mmol) in DCM was added DBU (0.65 g, 4.27 mmol) at 0° C. Then a solution of cap 12a (1 g, 6.41 mmol) in DCM was added. The reaction solution was allowed to stir at 25° C. for about 15 hours. After removal of the solvent, the crude was purified using column chromatography (100-200 mesh, Petroleum Ether/EtOAc=25/1 to 10/1) to provide cap 12c as a white solid (1 g, 65% yield). ¹H NMR (CDCl₃): δ 7.35 (s, 5H), 5.13 (s, 2H), 3.52-3.73 (m, 4H), 3.24 (br, 2H), 2.55-2.58 (m, 1H), 1.76-1.89 (m, 2H), 1.58-1.67 (m, 2H), 0.93-0.99 (m, 6H).

### Step 2

To a solution of cap 12c (1 g, 2.77 mmol) in MeOH was added Pd/C (0.2 g) carefully under H₂ (45 psi). Then the reaction mixture was allowed to stir at 25° C. for 3 hours. After completion of the reaction, the Pd/C was filtered and the solvent was removed to provide cap 12d as colorless oil (0.6 g, 95% yield). ¹H NMR (CDCl₃): δ 3.73 (s, 3H), 3.29 (s, 1H), 3.14-3.20 (m, 2H), 1.86-1.95 (m, 1H), 1.47-1.64 (m, 6H), 1.00-1.10 (m, 2H), 0.85-1.00 (m, 6H).

### Step 3

A mixture of cap 12d (6.8 g, 29.69 mmol) and NaOH (1.188 g, 29.69 mmol) in MeOH/H₂O (100 mil 20 mL) was allowed to stir at 25° C. for 1 hour. After completion of the reaction, the solvent was removed in vacuo and the remaining water solution was used directly for the next step (7.04 g, 100% yield). LC/MS: Anal. Calcd. For [M+H]⁺ C11H21NO3; 216.15. found 216.12.

### Step 4

To a solution of cap 12e (7.04 g, 29.69 mmol) in water (50 mL) was added sodium carbonate (3.148 g, 29.69 mmol) and methyl carbonochloridate (3.385 g, 35.63 mmol) dropwise at 0° C. Then the reaction mixture was allowed to stir at 25° C. for about 15 hours. MTBE was added. The organic phase was separated and discarded. The aqueous phase was added 1N HCl at 0° C. until pH=3. EtOAc was used to extract the product three times. The organic layer was then dried over Na₂SO₄ and concentrated in vacuo to provide cap 12f as a white solid (6 g, 74% yield). LC/MS: Anal. Calcd. For [M+H]⁺ C13H23NO5; 274.16. found 274.12.

### Step 5

Cap 12 was separated by SFC from cap 12f (2.5 g, 9.16 mmol) using the following condition as a white solid:

Column: Chiralpak AD-H 150×4.6 mm I.D.

Mobile phase: ethanol (0.05% DEA) in CO₂ from 5% to 40%

Flow rate: 2.5 mL/min

Wavelength: 220 nm

Cap 12 (0.9 g, 72% yield)¹H NMR (MeOD): δ 3.96 (d, T=4.4 Hz, 1H), 3.61 (s, 3H), 3.13-3.21 (m, 2H), 2.02-2.05 (m, 1H), 1.38-1.64 (m, 6H), 0.88-1.02 (m, 8H).

### Example 18

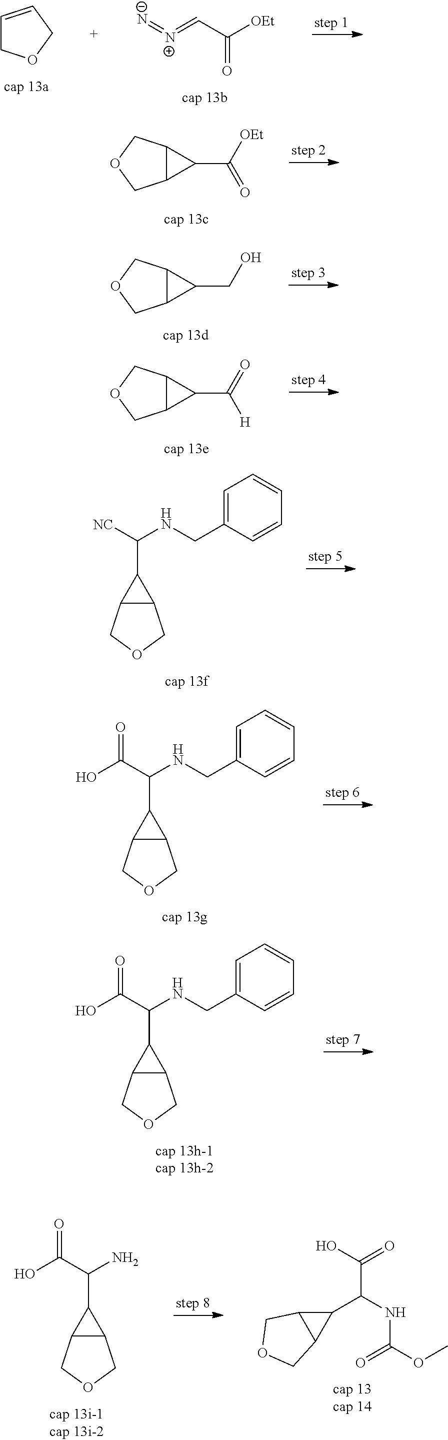

### Step 1

A solution of cap 13a (155 g, 2.22 mol) and Rh₂(OAc)₄ (1 g, 2.3 mmol) was added cap 13b (230 g, 2 mol) dropwise at 30-40° C. The solution was allowed to stir at 25° C. for 16 hours, and purified using chromatography (Petroleum Ether: EtOAc=5:1) to provide compound cap 13c (250 g, 79% yield). ¹H NMR (CDCl₃): δ 4.16-4.25 (m, 2H), 4.06-4.14 (m, 4H), 3.90 (d, J=9.0 Hz, 2H), 3.67-3.75 (m, 3H), 2.13 (br. s., 2H), 1.57 (t, J=2.9 Hz, 1H), 1.19-1.27 (m, 9H).

### Step 2

To a mixture of LiAlH₄ (91 g, 2.4 mol) in 1.2 L THF was added cap 13c (250 g, 1.6 mol) dropwise at 0° C. The mixture was allowed to stir at 25° C. for 30 minutes, and cooled to 0° C. 91 mL H₂O was added to the mixture at 0° C. dropwise. 91 mL 15% NaOH was added to the solution at 0° C. 273 mL H₂O and 600 g Na₂SO₄ was added to the mixture, and filtrated. The solid was washed with EtOAc. The combined organic extracts were concentrated in vacuo, purified using chromatography (Petroleum Ether:EtOAc=1:1) to provide cap 13d (90 g, 49% yield).

### Step 3

To a solution of oxalyl dichloride (1.4 mL, 13 mmol) in 20 mL DCM was added DMSO (1 mL, 1.9 mmol) at −78° C. under N₂, and stirred at −78° C. for 1 hour. The solution was added cap 13d (1.5 g, 13 mmol) at −78° C. and stirred at −78° C. for 1 hour. The solution was added Et₃N (4 g, 40 mmol) at −78° C. and stirred at −78° C. for 1 hour. The mixture was washed with 0.1N HCl, NaHCO₃, NaCl, dried over Na₂SO₄, concentrated in vacuo to provide the cap 13e (1.4 g, 100% yield).

### Step 4

A solution of cap 13e (1 g, 8.9 mmol), phenylmethanamine (0.87 g, 0.82 mmol) and TMSCN (0.80 g, 8.2 mmol) in 10 mL H₂O was allowed to stir at 25° C. for 16 hours. The solution was extracted with EtOAc. The combined organic extracts were dried over Na₂SO₄, concentrated in vacuo and purified using chromatography (Petroleum Ether:EtOAc=3:1) to provide the cap 13f (260 mg, 13% yield). ¹H NMR (CDCl₃): δ 7.15-7.43 (m, 5H), 4.02-4.17 (m, 1H), 3.79-3.92 (m, 3H), 3.62-3.71 (m, 2H), 3.32-3.50 (m, 1H), 1.60-1.78 (m, 3H), 1.18 (dt, J=6.6, 3.2 Hz, 1H). LC/MS: Anal. Calcd. For [M+H]⁺ C14H16N2O; 228.29. found: 229.

### Step 5

A solution of cap 13f (260 mg, 1.1 mmol) in 10 mL HCl (60 mmol) was allowed to stir at 90-100° C. for 72 hours. The solution was purified using Pre-HPLC to provide cap 13g (100 mg, 35% yield). LC/MS: Anal. Calcd. For [M+H]⁺ C14H17NO3; 247.29. found: 248.

### Step 6

Compound cap 13h-1 & compound cap 13h-2 was separated from cap 13g by SFC using the following method:

Column: Chiralpak AD-H 250×4.6 mm I.D., Sum

Mobile phase: methanol (0.05% DEA) in CO₂ from 5% to 40%

Flow rate: 2.35 mL/min

Wavelength: 220 nm

### Step 7

A solution of compound cap 13h-1 (300 mg, 1.2 mol) and 3 mL 12 N HCl in 50 mL MeOH was added 30 mg Pd/C and stirred at 25° C. for 16 hours under H₂ (50 psi). The mixture was filtered and the filtrate was concentrated in vacuo to provide the compound cap 13i-1 (180 mg, 95% yield). LC/MS: Anal. Calcd. For [M+H]⁺ C7H11NO3; 157.17. found: 158.

Cap 13i-2 was prepared using the same method (180 mg, 95% yield). LC/MS: Anal. Calcd. For [M+H]⁺ C7H11NO3; 157.17. found: 158.

### Step 8

A solution of cap 13i-1 (189 mg 1.2 mmol), LiOH (76 mg, 1.8 mmol) and Na₂CO₃ (128 mg, 1.2 mmol) in 10 mL H₂O was allowed to stir at 25° C. for 2 hours and added methyl chloroformate (120 mg, 1.3 mmol) at 25° C. and stirred for 4 hours. The solution was extracted with DCM. The water layer was added 0.1 N HCl to pH=2. The solution was extracted with EtOAc. The combined organic extracts were dried over Na₂SO₄, concentrated in vacuo to provide the compound cap 13 (120 mg, 46% yield). ¹H NMR (CDCl₃): δ 5.35 (d, J=6.26 Hz, 1H), 3.88 (t, J=8.41 Hz, 2 H), 3.68-3.74 (m, 5H), 1.82 (d, J=16.43 Hz, 2H), 1.08 (d, J=8.61 Hz, 1H). LC/MS: Anal. Calcd. For [M+H]⁺ C9H13NO5; 215.20. found: 216.

Cap 14 was prepared from cap 13i-2 by the same method (120 mg, 46% yield). ¹H NMR (CDCl₃): δ 5.32 (br. s., 1H), 3.88 (t, J=8.4 Hz, 2H), 3.70 (s, 5H), 1.81 (d, J=15.7 Hz, 2H), 1.03-1.12 (m, 1H). LC/MS: Anal. Calcd. For [M+H]⁺ C9H13NO5: 215.20. found: 216.

### Example 19

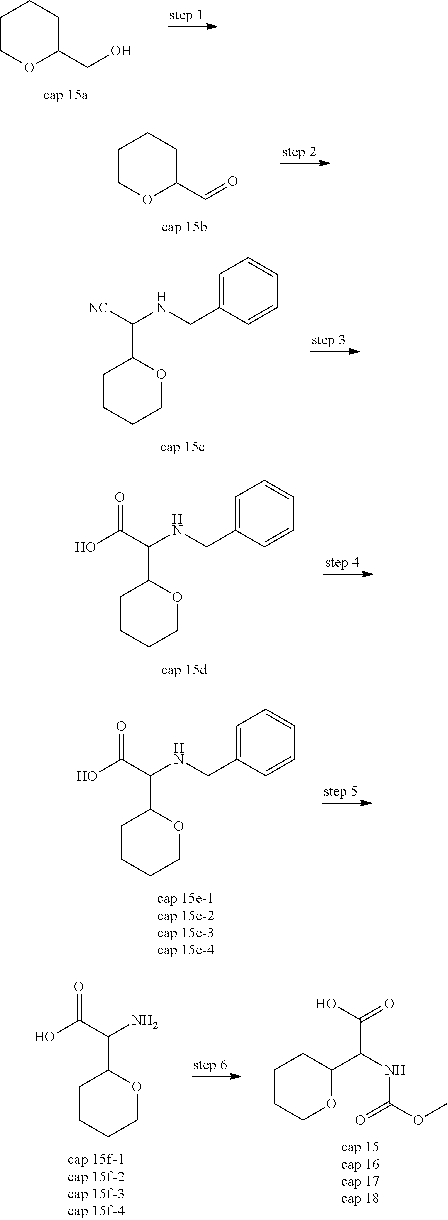

### Step 1

To a solution of oxalyl dichloride (9 mL, 103 mmol) in 20 mL DCM was added DMSO (7 mL, 130 mmol) at −78° C. under N₂ atmosphere, and stirred at −78° C. for 1 hour. The solution was added cap 15a (10 g, 86 mmol) at −78° C. and stirred at −78° C. for 1 hour. The solution was added Et₃N (26 g, 260 mmol) at −78° C. and stirred at −78° C. for 1 hour. The mixture was washed with 0.1N HCl, NaHCO₃ aqueous, brine, dried over Na₂SO₄, concentrated in vacuo to provide the compound cap 15b (9.8 g, 97% yield).

### Step 2

A solution of cap 15b (9.5 g, 83 mmol), phenylmethanamine (8.26 g, 83 mmol) and TMSCN (8.92 g, 83 mmol) in 100 mL H₂O was allowed to stir at 25° C. for 16 hours. The solution was extracted with EtOAc. The combined organic extracts were dried over Na₂SO₄, concentrated in vacuo and purified using chromatography (Petroleum Ether:EtOAc=5:1) to provide cap 15c. (10 g, 52% yield). ¹H NMR (DMSO): δ 7.25-7.36 (m, 5H), 3.80-3.93 (m, 2H), 3.57-3.72 (m, 2H), 3.46 (d, J=5.9 Hz, 1H), 3.32-3.39 (m, 1H), 2.82-3.00 (m, 1H), 1.70-1.82 (m, 1H), 1.56-1.65 (m, 1H), 1.42 (br. s., 5H). LC/MS: Anal. Calcd. For [M+H]⁺ C14H18N2O: 230.31. found: 231.

### Step 3

A solution of cap 15c (1 g, 4.3 mmol) in 15 mL HCl (90 mmol) was allowed to stir at 90-100° C. for 72 hours. The solution was purified using Pre-HPLC to provide the compound cap 15d (100 mg, 9.2% yield). ¹H NMR (DMSO): δ 7.37-7.50 (m, 5H), 4.16-4.34 (m, 2H), 3.99 (d, J=11.3 Hz, 1H), 3.70-3.84 (m, 2H), 3.38-3.51 (m, 1H), 1.89 (br. s., 1H), 1.43-1.63 (m, 5H). LC/MS: Anal. Calcd. For [M+H]⁺ C14H19NO3; 249.31. found: 250.

### Step 4

Cap 15e-1, cap 15e-2, cap 15e-3 & cap 15e-4 were separated from Compound cap 15d by SFC using the following method.

Column: Chiralpak AS-H 250×4.6 mm I.D., Sum

Mobile phase: methanol (0.05% DEA) in CO₂ from 5% to 40%

Flow rate: 2.35 mL/min

Wavelength: 220 nm

### Step 5

A solution of compound cap 15e-1 (90 mg, 3.6 mmol) in 50 mL MeOH was added 9 mg Pd/C and stirred at 25° C. for 16 hours under H₂ (50 psi). The mixture was filtered and the filtrate was concentrated in vacuo to provide cap 15f-1 (50 mg, 100% yield). LC/MS: Anal. Calcd. For [M+H]⁺ C7H13NO3; 159.18. found: 160.

Cap 15f-2 was prepared using the same method from Cap 15e-2 (570 mg, 100% yield). LC/MS: Anal. Calcd. For [M+H]⁺ C7H13NO3; 159.18. found: 160.

Cap 15f-3 was prepared using the same method from Cap 15e-3 (570 mg, 100% yield). LC/MS: Anal. Calcd. For [M+H]⁺ C7H13NO3; 159.18. found: 160.

Cap 15f-4 was prepared using the same method from Cap 15e-4 (50 mg, 100% yield). LC/MS: Anal. Calcd. For [M+H]⁺ C7H13NO3; 159.18. found: 160.

### Step 6

A solution of compound cap 15f-1 (55 mg 0.35 mmol), LiOH (53 mg, 1.27 mmol) in 5 mL H₂O was allowed to stir at 25° C. for 2 hours and added methyl chloroformate (40 mg, 0.35 mmol) at 25° C. and stirred for 4 hours. The solution was extracted with DCM. The water layer was added 0.1 N HCl to pH=2. The solution was extracted with EtOAc. The combined organic extracts were dried over Na₂SO₄, concentrated in vacuo to provide cap 15 (45 mg, 60% yield). ¹H NMR (DMSO) δ: 7.39 (d, J=9.0 Hz, 1H), 3.98 (d, J=8.2 Hz, 1H), 3.83 (d, J=10.6 Hz, 1H), 3.51 (s, 5H), 1.74 (br. s., 1H), 1.31-1.57 (m, 2H). LC/MS: Anal. Calcd. For [M+H]⁺ C9H15NO5; 217.22. found: 217.

Cap 16 was prepared using the same method from Cap 15f-2 (300 mg, 36% yield). ¹H NMR (DMSO) δ: 7.00 (d, J=9.0 Hz, 1H), 4.03 (d, J=8.2 Hz, 1H), 3.85 (d, J=10.6 Hz, 1H), 3.72 (s, 5H), 1.74 (br. s., 1H), 1.31-1.57 (m, 2H). LC/MS: Anal. Calcd. For [M+H]⁺ C9H15NO5; 217.22. found: 217.

Cap 17 was prepared using the same method from Cap 15f-3 (300 mg, 36% yield). ¹H NMR (DMSO) δ: 7.00 (d, J=9.0 Hz, 1H), 4.03 (d, J=8.2 Hz, 1H), 3.85 (d, J=10.6 Hz, 1H), 3.72 (s, 5H), 1.74 (br. s., 1H), 1.31-1.57 (m, 2H). LC/MS: Anal. Calcd. For [M+H]⁺ C9H15NO5; 217.22. found: 217.

Cap 18 was prepared using the same method from Cap 15f-4 (45 mg, 60% yield). ¹H NMR (DMSO) δ: 7.39 (d, J=8.6 Hz, 1H), 3.98 (d, J=7.8 Hz, 1H), 3.83 (d, J=11.7 Hz, 1H), 3.51 (s, 4H), 1.75 (br. s., 1H), 1.29-1.55 (m, 5H). LC/MS: Anal. Calcd. For [M+H]⁺ C9H15NO5; 217.22. found: 217.

### Example 20

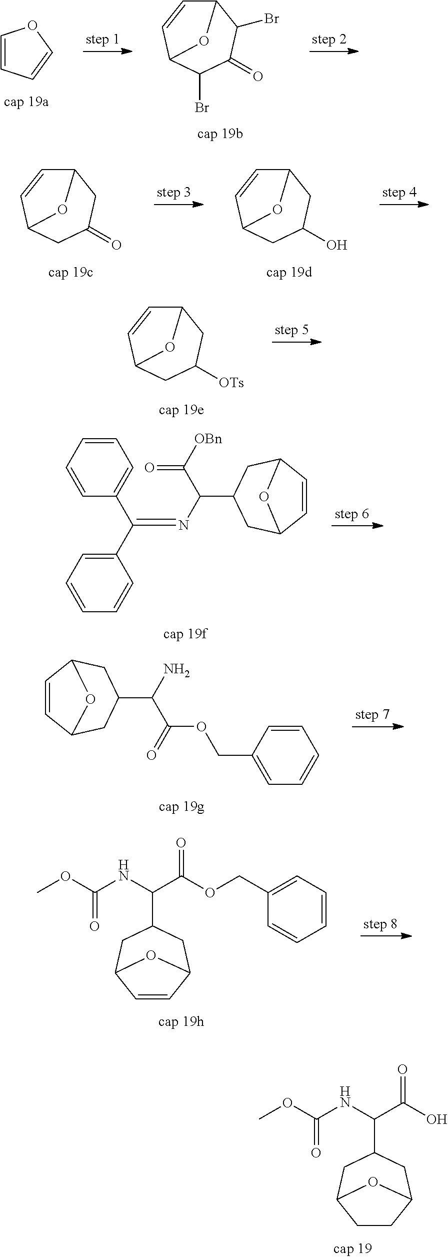

### Step 1

To a solution of cap 19a (1.3 g, 18.77 mmol) and Zn powder (2 g, 31 mmol) in THF (3 mL) was added 1,1,3,3-tetrabromopropan-2-one (10.5 g, 28 mmol) and triethyl borate (5.48 g, 38 mmol) dropwise at 25° C. during 1 hour in the dark. The resulting dark brown mixture was allowed to stir at 25° C. for 17 hours. The mixture was cooled to −15° C., to the mixture was added 30 mL of H₂O and stirred for additional 30 minutes, extracted with EtOAc. The combined organic phases were dried. After filtration, the solvent was removed to provide cap 19b (26.1 g, 100% yield).

### Step 2

To a solution of cap 19b (26.1 g, 93 mmol) in MeOH (30 mL) was added Zn powder (36.3 g, 558 mmol), CuCl (4.6 g, 46.5 mmol) and NH₄Cl (34.5 g, 0.64 mol) in MeOH (80 mL). The reaction mixture was maintained below 15° C. during addition. The mixture was allowed to stir at 25° C. for 19 hours, and then extracted with EtOAc. The combined organic phases were dried. After filtration, the solvent was removed by evaporation to provide cap 19c (3.5 g, 30.4% yield).

### Step 3

To a solution of cap 19c (700 mg, 5.64 mmol) in THF (20 mL) was added L-selectride (11.3 mL, 11.3 mmol) at −78° C. over 100 min. The mixture was allowed to stir under N₂ at −78° C. for 1 h, and then warmed to 25° C. for 12 hours. The mixture was cooled to 0° C., 1N NaOH (5 mL) was added and then 5 mL of H₂O was added. The mixture was allowed to stir at 25° C. for 1 hour, quenched with 3N HCl, the residue obtained was portioned between water and EtOAc. The organic layer was washed with brine, dried over sodium sulfate, filtered and concentrated in vacuo to provide cap 19d (320 mg, 45.4% yield).

### Step 4

To a solution of cap 19d (320 mg, 2.56 mmol) and pyridine (820 mg, 10.24 mmol) in DCM (20 mL) was added TsCl (973 mg, 5.12 mmol) at 0° C. The mixture was allowed to stir under N₂ at 25° C. for 12 hours before it was poured into water and extracted with DCM. The organic layer was washed with brine, dried over sodium sulfate. After filtration and concentration, the residue obtained was purified using column (eluting with Petroleum Ether:EtOAc=5:1) to provide cap 19e (330 mg, 45.9% yield).

### Step 5

A microwave tube was charged with cap 19e (330 mg, 1.18 mmol) and benzyl 2-(diphenylmethyleneamino) acetate (466 mg, 1.4 mmol) in toluene (15 mL) was added LiHMDS (1.5 mL, 1.5 mmol) dropwise under N₂. The mixture was allowed to stir at 100° C. under microwave for 4 hours, before it was poured into water and extracted with EtOAc. The organic layer was washed with brine, dried over sodium sulfate. After filtration and concentration, the residue obtained was purified using column (eluting with Petroleum Ether:EtOAc=5:1) to provide cap 19f (330 mg, 65% yield).

### Step 6

To a solution of cap 19f (330 mg, 0.75 mmol) in THF (20 mL) was added 2N HCl (5 mL) at 0° C. The mixture was allowed to stir under N₂ at 25° C. for 2 hours before it was poured into saturated NaHCO₃ and extracted with DCM. The organic layer was washed with brine, dried over sodium sulfate. After filtration and concentration, it was got cap 19g (150 mg, 72.8% yield).

### Step 7

To a solution of cap 19g (150 mg, 0.55 mmol) and Et₃N (221 mg, 2.2 mmol) in DCM (10 mL) was added MocCl (96 mg, 0.95 mmol) at 0° C. The mixture was allowed to stir under N₂ at 25° C. for 12 hours before it was poured into water and extracted with DCM. The organic layer was washed with brine, dried over sodium sulfate. After filtration and concentration, the residue obtained was purified using column (eluting with Petroleum Ether:EtOAc=1:1) to provide cap 19h (60 mg, 60% yield).

### Step 8

To a solution of cap 19h (60 mg, 0.18 mmol) in MeOH (10 mL) was added Pd/C (10 mg, 0.03 mmol). The mixture was allowed to stir at 25° C. under 40 psi of H₂ for 12 hours before evaporated to remove the solvent to provide compound cap 19 (40 mg, 93% yield). ¹H NMR (MeOD): δ 4.35 (s, 2H), 3.95 (d, J=4.4 Hz, 1H), 3.62 (s, 3H), 1.85-1.95 (m, 2H), 1.70-1.80 (m, 2H), 1.39-1.60 (m, 4H), 1.23-1.35 (m, 1H), 1.16 (t, J=7.2 Hz, 1H).

### Example 21

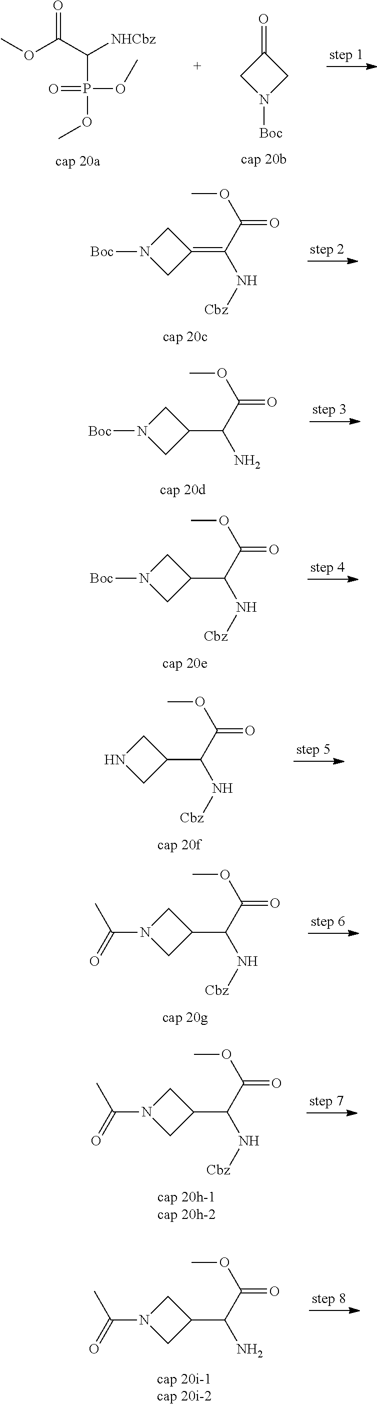

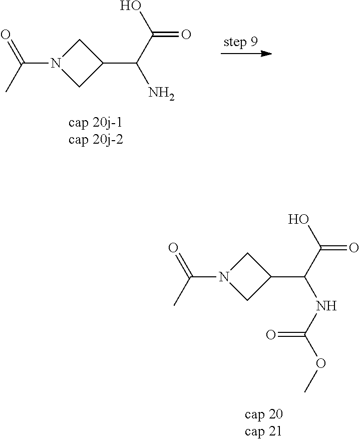 [SMILES: CC(=O)N1CC(C(N)C(=O)O)C1 ; COC(=O)NC(C(=O)O)C1CN(C(C)=O)C1]

### Step 1

A solution of cap 20a (19.8 g, 60 mmol) in THF (100 mL) was added tetramethylguanidine (7.6 g, 66 mmol) at −20° C. dropwise. The solution was allowed to stir at −20° C. for 1 hour. Cap 20b (11.2 g, 66 mmol) was added at −20° C. dropwise and stirred at 20° C. for about 15 hours. The solution was added H₂O (100 mL) and extracted with EtOAc. The combined organic phase was washed with 1 N HCl, Na₂CO₃ and brine, dried over Na₂SO₄, concentrated in vacuo and purified using chromatography to provide cap 20c as a colorless oil (20.5 g, 89% yield). ¹H NMR (CDCl₃): δ 7.34-7.36 (m, 5H), 5.12 (s, 2H), 4.72-4.75 (m, 4H), 3.79 (s, 3H), 1.45 (s, 9H).

### Step 2

To a stirred solution of cap 20c (20.5 g, 54.6 mmol) in methanol (100 mL) was added 10 percent Pd/C (2 g). The mixture was allowed to stir under 45 Psi H₂ pressure at 45° C. for about 15 hours and filtered through celite. The filtrate was concentrated in vacuo to provide cap 20d (12 g, 90% yield). ¹H NMR (CDCl₃): δ 3.90-3.97 (m, 2H), 3.73-3.801 (m, 2H), 3.68 (s, 3H), 3.51 (d, J=8.8 Hz, 2H), 2.62-2.68 (m, 1H), 1.39 (s, 9H).

### Step 3

To a solution of cap 20d (12 g, 50 mmol) and NaHCO₃ (8.4 g, 55 mmol) in 150 mL THF/H₂O (1:2) was added CbzCl (9.4 g, 55 mmol) at 0° C. and stirred at 25° C. for 10 hours. The mixture was extracted with EtOAc. The combined organic phase was dried over Na₂SO₄, and concentrated in vacuo, which used in the next step directly (18 g crude).

### Step 4

Cap 20e (10 g crude) was treated with 4 N methanolic HCl (100 mL), and stirred at 25° C. for 1 hour. The mixture was concentrated in vacuo, which used in the next step directly (7.0 g crude). MS (ESI) m/e (M+H⁺): 279.

### Step 5

To a solution of cap 20f (7.0 g crude) and Na₂CO₃ (6.4 g, 60 mmol) in 150 mL THF/H₂O (½) was added AcCl (2.5 g, 31 mmol) at 0° C. and stirred at 25° C. for 10 hours. The mixture was extracted with EtOAc. The combined organic phase was dried over Na₂SO₄ and concentrated in vacuo. The residue obtained was purified using chromatography to provide cap 20g (6.0 g, 69% yield for 3 steps). ¹H NMR (CDCl₃): δ 7.32 (bs, 5H), 5.56-5.61 (m, 1H), 5.05-5.12 (m, 2H), 4.45-4.57 (m, 1H), 3.77-4.15 (m, 4H), 3.71 (s, 3H), 2.88-2.95 (m, 1H), 1.77-1.79 (m, 3H).

### Step 6

Cap 20g (6.0 g, 18.7 mmol) was separated by SFC to provide two enantiomers cap 20h-1 (2.4 g, 80% yield) and cap 20h-2 (2.6 g, 86% yield).

Column: Chiralpak AD-H 150×4.6 mm I.D.

Mobile phase: ethanol (0.05% DEA) in CO₂ from 5% to 40%

Flow rate: 2.35 mL/min

Wavelength: 220 nm

Cap 20h-1 ¹H NMR (CDCl3): δ 7.32 (bs, 5H), 5.63-5.66 (m, 1H), 5.05-5.13 (m, 2H), 4.52-4.57 (m, 1H), 3.67-4.14 (m, 7H), 2.87-2.97 (m, 1H), 1.77-1.79 (m, 3H).

Cap 20h-2 ¹H NMR (CDCl3): δ 7.32 (bs, 5H), 5.60-5.66 (m, 1H), 5.05-5.12 (m, 2H), 4.54-4.57 (m, 1H), 3.67-4.12 (m, 7H), 2.87-2.97 (m, 1H), 1.77-1.79 (m, 3H).

### Step 6

To a solution of cap 20h-1 (2.6 g, 8.27 mmol) in methanol (10 mL) was added 10 percent Pd/C (10%, 0.2 g). The mixture was allowed to stir under 45 Psi H₂ pressure at 45° C. for about 15 hours and filtered through celite. The filtrate was concentrated in vacuo to provide cap 20i-1 (1.4 g, 91% yield). ¹H NMR (MeOD): δ 4.20-4.25 (m, 1H), 4.08-4.10 (m, 1H), 3.97-4.05 (m, 1H), 3.82-3.86 (m, 1H), 3.70 (s, 3H), 3.59-3.62 (m, 1H), 2.78-2.88 (m, 1H), 1.82-1.83 (m, 3H). Compound cap 20i-2 was prepared using the same method from cap 20h-2 (1.7 g, 100% yield). ¹H NMR (MeOD): δ 4.20-4.25 (m, 1H), 4.07-4.10 (m, 1H), 3.98-4.05 (m, 1H), 3.82-3.86 (m, 1H), 3.70 (s, 3H), 3.60-3.62 (m, 1H), 2.79-2.88 (m, 1H), 1.82-1.83 (m, 3H).

### Step 7

A mixture of cap 20i-1 (558 mg, 3 mmol) and LiOH (190 mg, 4.5 mmol) in 25 mL of THF/H₂O (5:1) was allowed to stir at 25° C. for 4 hours. The mixture was used in the next step directly.

Compound cap 20j-2 was prepared using the same method from cap 20i-2.

### Step 8

Methyl chloroformate (302 mg, 3.2 mmol) was added to crude cap 20j-1 in CH₂Cl₂ at 0° C., and stirred at 25° C. for 2 hours. The mixture was adjusted pH to 6 with 1 N HCl, and concentrated in vacuo and purified using Pre-HPLC to provide cap 20 (210 mg, 30% yield over 2 steps). ¹H NMR (MeOD): δ 4.36-4.38 (m, 1H), 4.20-4.32 (m, 1H), 3.97-4.15 (m, 2H), 3.86-3.90 (m, 0.5H), 3.75-3.79 (m, 0.5 H), 3.65 (s, 3H), 3.00-3.07 (m, 1H), 1.83-1.84 (m, 3H).

Cap 21 was prepared using the same method from cap 20j-2 (150 mg, 22% yield over 2 steps). ¹H NMR (MeOD): δ 4.36-4.38 (m, 1H), 4.20-4.32 (m, 1H), 3.97-4.15 (m, 2H), 3.86-3.90 (m, 0.5H), 3.75-3.79 (m, 0.5H), 3.65 (s, 3H), 3.00-3.07 (m, 1H), 1.84-1.85 (m, 3H).

### Example 22

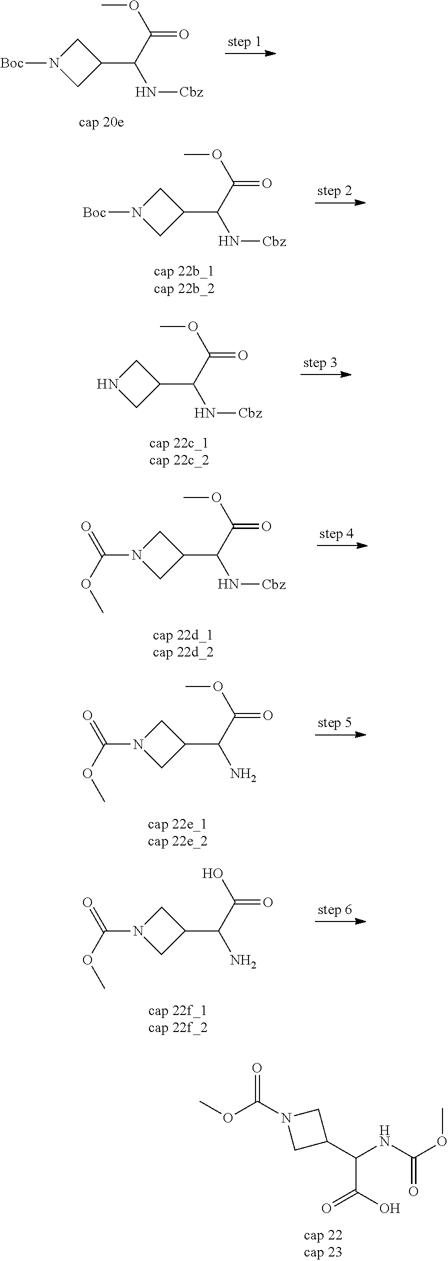

### Step 1

Compound cap 20e was prepared in Example 21

Compound cap 20e (6.0 g, 18.7 mmol) was separated by SFC using the following method to provide two enantiomers cap 22b-1 (1.6 g, 59.3% yield) and cap 22b-2 (1.4 g, 51.8% yield).

Column: Chiralpak AD-H 250×4.6 mm I.D., Sum

Mobile phase: methanol (0.05% DEA) in CO2 from 5% to 40%

Flow rate: 2.35 mL/min

Wavelength: 220 nm

### Step 2

Cap 22b-1 (1.6 g, 4 mmol) was treated with 4 N methanolic HCl (100 mL), and stirred at 25° C. for 1 hour. The mixture was concentrated in vacuo, which used in the next step directly (1.2 g, 100% yield).

Cap 22c-2 was prepared with the same method from cap 22b-2 (1.2 g, 100% yield).

### Step 3

To a solution of crude cap 22c-1 (0.5 g) and Na₂CO₃ (0.53 g, 5 mmol) in 50 mL THF/H₂O (1:2) was added methyl chloroformate (0.26 g, 2.8 mmol) at 0° C. and stirred at 25° C. for 10 hours. The mixture was extracted with EtOAc. The combined organic phases were dried over Na₂SO₄ and concentrated in vacuo. The residue obtained was purified using chromatography to provide cap 22d-1 (0.6 g, 100% yield).

Cap 22d-2 was prepared with the same method from cap 22c-2 (0.6 g, 100% yield).

### Step 4

To a stirred solution of cap 22d-1 (0.8 g, 2.37 mmol) in methanol (10 mL) was added Pd/C (10%, 0.2 g). The mixture was allowed to stir under 45 Psi H₂ pressure at 45° C. for about 15 hours and filtered through celite. The filtrate was concentrated in vacuo to provide cap 22e-1 (0.4 g, 83.3% yield).

Cap 22e-2 was prepared with the same method from cap 22d-2 (0.4 g, 83.3% yield).

### Step 5

A mixture of cap 22e-1 (400 mg, 2 mmol) and LiOH (120 mg, 3 mmol) in 12 mL of THF/H₂O (5:1) was allowed to stir at 25° C. for 4 hours. The mixture was used in the next step directly.

Cap 22f-2 was prepared with the same method from cap 22e-2.

### Step 6

Methyl chloroformate (220 mg, 2 mmol) was added to cap 22f-1 in an ice-water bath, and stirred at 25° C. for 2 hours. The mixture was adjusted pH to 6 with 1 N HCl, and concentrated in vacuo and purified using Prep-HPLC to provide cap 22 (300 mg, 61.2% yield). LC/MS: Anal. Calcd. For [M+H]⁺ C9H14N2O6; 247.09. found: 247.1.

Cap 23 was prepared with the same method from cap 22f-2 (300 mg, 61.2% yield). LC/MS: Anal. Calcd. For [M+H]⁺ C9H14N2O6; 247.09. found: 247.1.

### Example 23

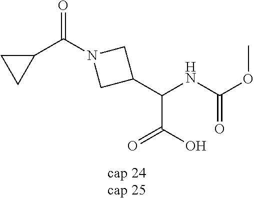 [SMILES: COC(=O)NC(C(=O)O)C1CN(C(=O)C2CC2)C1]

Cap 24 was prepared following the same method of cap 22 from cap 22c_1 (300 mg, 58.8% yield). LC/MS: Anal. Calcd. For [M+H]⁺ C11H16N2O5; 257.10. found: 257.1.

Cap 25 was prepared following the same method of cap 23 from cap 22c_2 (300 mg, 58.8% yield). LC/MS: Anal. Calcd. For [M+H]⁺ C11H16N2O5; 257.10. found: 257.1.

### Example 24

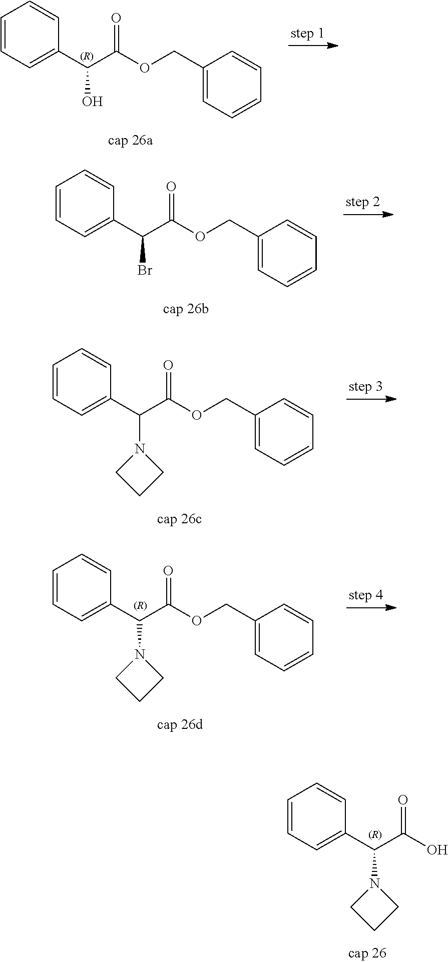

### Step 1

To a solution of compound cap 26a (4 g, 16.5 mmol) in THF (30 mL) was added PBr₃ (4.9 g, 18.2 mmol) dropwise at 0° C. Then it was warmed to 25° C. and the mixture was allowed to stir for 2 hours. The mixture was washed with water, extracted with EtOAc, and the column chromatography was given cap 26b (Petroleum Ether/EtOAc=10:1) (1.5 g, 30% yield).

### Step 2

To a solution of cap 26b (1.5 g, 4.9 mmol) in THF (10 mL) was added TEA (992 mg, 9.8 mmol), followed by TBAI (362 mg, 0.98 mmol). The reaction mixture was allowed to stir at 25° C. for 5 min and then a solution of HCl salt (550 mg, 5.88 mmol) in THF (5 mL) was added. The mixture was allowed to stir for 16 hours. Extracted with EtOAc, washed with water, dried, filtered and concentrated the solvent under reduced pressure. The residue obtained was purified on silica gel column (Petroleum Ether/EtOAc=1:5), given a mixture of diastereomers (1:1) cap 26c (780 mg, yield 56%).

### Step 3

Cap 26d was separated from cap 26c using the following method.

Column: Chiralpak AD-H 250×4.6 mm I.D., Sum

Mobile phase: ethanol (0.05% DEA) in CO₂ from 5% to 40%

Flow rate: 2.35 mL/min

Wavelength: 220 nm

### Step 4

A mixture of cap 26d (240 mg, 0.85 mmol) and dry Pd/C (10%, 25 mg) in MeOH (18 mL) was allowed to stir under 45 psi of H₂ at 45° C. for about 15 hours. Filtered through celite and reduced pressure. Pre-HPLC was given cap 26 (120 mg, yield 74%). LC/MS: Anal. Calcd. For [M+H]⁺ C11H13NO2; 192.09. found: 192.1.

### Example 25

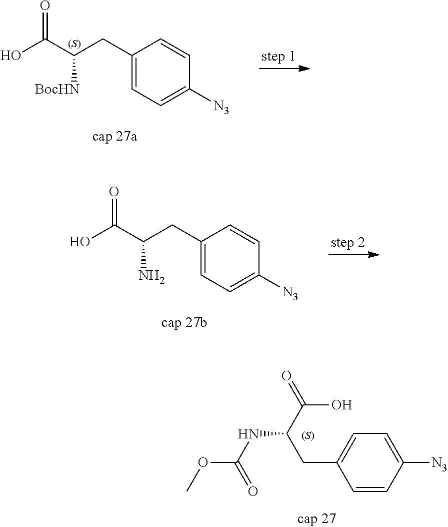

### Step 1

Cap 27a (1 g, 3.27 mmol) was dissolved in 20 mL of DCM, TFA (0.6 mL) was added in dropwised. Then the mixture was allowed to stir at 35° C. for 5 hours. After that, the mixture was concentrated in vacuo to provide cap 27b (0.673 g, 100% yield). LC/MS: Anal. Calcd. For [M+H]⁺ C9H10N4O2; 207.09. found: 207.1.

### Step 2

Cap 27b (0.673 g, 3.27 mmol) and Na₂CO₃ (0.52 g, 4.9 mmol) were dissolved in 16 mL of THF/H₂O (1:1). The mixture was cooled to 0° C., Methyl chloroformate (0.369 g, 3.92 mmol) was added in dropwised, then the mixture was allowed to stir at 25° C. for 2 hours. After that, the mixture was washed with water (20 mL) and extracted with EtOAc (30 mL), washed with brine and dried over anhydrous sodium sulfate. After being concentrated in vacuo, the residue obtained was transferred to purification team to do HPLC purification to provide cap 27 (0.42 g, 48.5% yield). LC/MS: Anal. Calcd. For [M+H]⁺ C11H12N4O4; 265.09. found: 265.1.

### Example 26

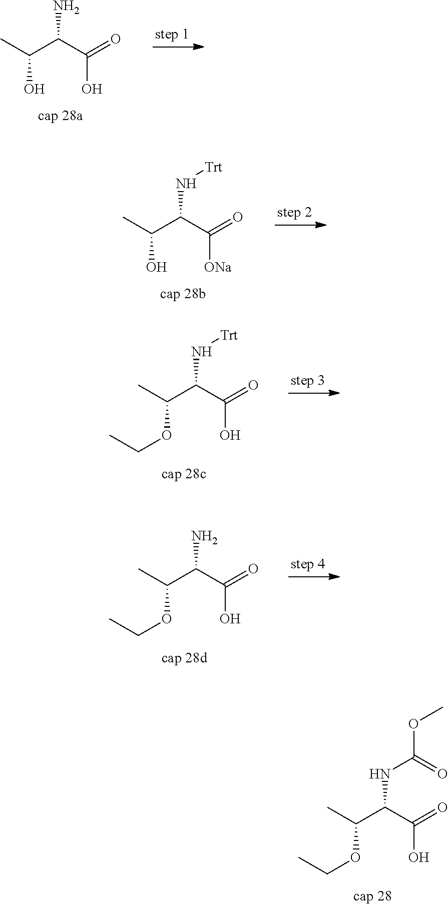

### Step 1

Cap 28a (21.25 g, 0.178 mol) was suspended in dry dichloromethane (315 mL) and trimethylsilyl chloride (79.3 mL) was added and the stirred reaction mixture was heated to reflux for 20 minutes. After cooling at 20° C., a solution of triethylamine (87.1 mL) in dry dichloromethane (180 mL) was added and the mixture heated to reflux for 45 minutes then cooled to 0° C. Anhydrous methanol (10.8 mL) in dry dichloromethane (45 mL) was then added dropwise and the mixture was cooled to 25° C. Triethylamine (24.9 mL) followed by trityl chloride (49.8 g) was added over a total of 15 minutes and the reaction mixture stirred for 24 hours at room temperature. Methanol (36.2 mL) followed by triethylamine (24.9 mL) was added and the mixture stirred for a further hour, then heated to reflux for 30 minutes. Evaporation of the solvent under reduced pressure gave an oily residue which was partitioned between aqueous 5% citric acid (900 mL) and ether (900 mL)/ethylacetate (500 mL). 1N aqueous sodium hydroxide solution (360 mL) was added to the organic layer and the solid which precipitated was filtered off and dried in vacuo to provide N-tritylallothreonine sodium salt (23.2 g).

### Step 2

To a solution of cap 28b (7.22 g, 20.0 mmol) in THF (30 mL) was added NaH (880 mg, 22 mmol) and the mixture was allowed to stir for 15 min 0° C. Then iodoethane (3.00 g, 20.0 mmol) was added and the mixture was allowed to stir for about 15 hours. The mixture was poured into water and acidified by 1N HCl. The mixture was extracted with EtOAc. The organic layer was removed in vacco. The residue obtained was purified using chromatography to provide cap 28c (5.2 g, 67% yield).

### Step 3

To a solution of cap 28c (5.2 g, 13.4 mmol) in DCM (90 mL) was added TFA (40 mL) and the mixture was allowed to stir at 25° C. for 2 hours. Then the mixture was concentrated in vacuo and used in the next reaction directly.

### Step 4

To a solution of cap 28d (1.5 g, 10.0 mmol) in H₂O (30 mL) was added Na₂CO₃ (2.12 g, 20 mmol) and the mixture was allowed to stir for 15 min 0° C. Then methyl carbonochloridate (1.0 g, 10.0 mmol) was added and the mixture was allowed to stir for 2 hours. The mixture acidified by 1N HCl. The mixture was extracted with EtOAc. The organic layer was removed in vacco to provide cap 28 (1.67 g, 81% yield).

### Example 27

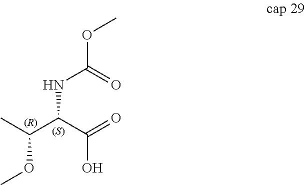

Cap 29 was prepared as described in Example 1 of International Publication No. WO 2012/041014.

### Example 28

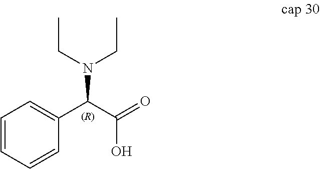

Cap 30 was prepared as described in Example 3 of International Publication No. WO 2012/041014.

### Example 29

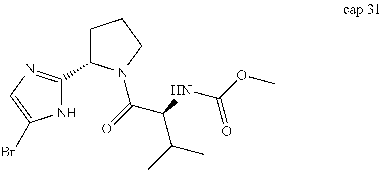 [SMILES: COC(=O)N[C@H](C(=O)N1CCC[C@H]1c1ncc(Br)[nH]1)C(C)C]

Cap 31 was prepared using the methods described in International Publication No. WO2012/040923.

### Example 30

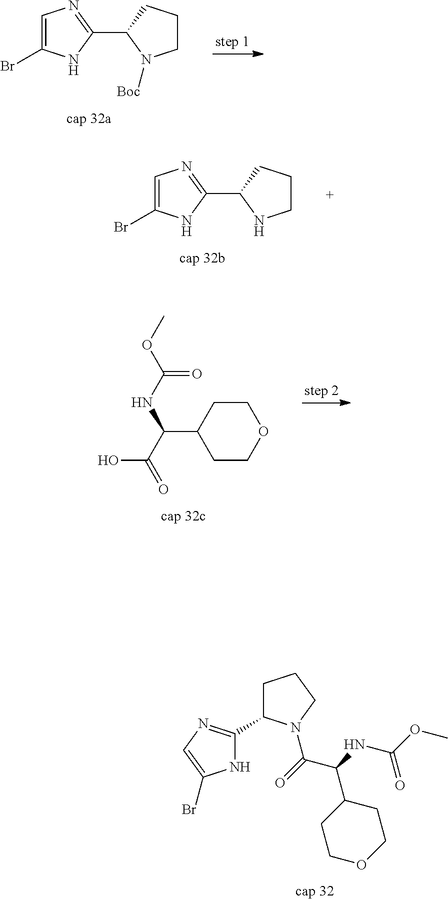 [SMILES: CC(C)(C)OC(=O)N1CCC[C@H]1c1ncc(Br)[nH]1 ; Brc1cnc([C@@H]2CCCN2)[nH]1 ; COC(=O)N[C@H](C(=O)O)C1CCOCC1 ; COC(=O)N[C@H](C(=O)N1CCC[C@H]1c1ncc(Br)[nH]1)C1CCOCC1]

### Step 1

Cap 32a was prepared as described in Example 7 of International Publication No. WO 2012/040923. Cap 32a (50 g, 0.16 mmol) was added into TFA/DCM (1:1, 10 mL). The mixture was allowed to stir at 25° C. for 2 hours; then concentrated in vacuo and dried under high vacuum to provide to desired product cap 32b (34.4 g, 100% yield). LC/MS: Anal. Calcd. For [M+H]⁺ C7H10BrN3; 216.01. found 216.1.

### Step 2

Cap 32c was prepared as described in International Publication No. WO 2012/040923. To a mixture of cap 32b (1.9 g, 9 mmol), cap 32c (1.9 g, 9 mmol) and DIPEA (4 mL) in CH₂Cl₂ (5 mL) was added HATU (3.5 g, 9 mmol). The resulting reaction was allowed to stir at 25° C. for 2 hours. The reaction mixture was concentrated in vacuo, then purified using Flash column chromatography on silica gel (eluent:petroleum ether/ethyl acetate=5:1 to 1:2) to provide cap 32 (2 g, 54.1% yield). LC/MS: Anal. Calcd. For [M+H]⁺ C16H23BrN4O4; 415.09, 417.09. found 415.1, 417.1.

### Example 31

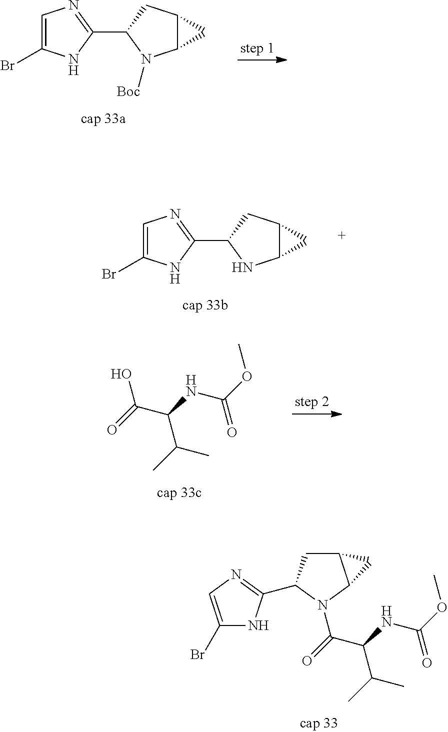 [SMILES: CC(C)(C)OC(=O)N1[C@H](c2ncc(Br)[nH]2)C[C@@H]2C[C@@H]21 ; Brc1cnc([C@@H]2C[C@@H]3C[C@@H]3N2)[nH]1 ; COC(=O)N[C@H](C(=O)O)C(C)C ; COC(=O)N[C@H](C(=O)N1[C@H](c2ncc(Br)[nH]2)C[C@@H]2C[C@@H]21)C(C)C]

### Step 1

Cap 33a was prepared as described in Example 12A of International Publication No. WO 2012/041014.

Cap 33a (2 g, 6 mmol) was added into TFA/DCM (1:1, 10 mL). Then the mixture was allowed to stir at 25° C. for 2 hours; then concentrated in vacuo and dried under high vacuum to provide to desired product cap 33b (1.4 g, 100% yield). LC/MS: Anal. Calcd. For [M+H]⁺ C8H10BrN3; 227.01. found 228.1, 230.1.

### Step 2

Compound cap 33c was prepared as described in Example 1 of International Publication No. WO 2012/041014. To a mixture of cap 33b (1.4 g, 6 mmol), cap 33c (1.085 g, 6.2 mmol) and DIPEA (4 mL) in CH₂Cl₂ (30 mL) was added HATU (2.36 mg, 6.2 mmol). The resulting mixture was allowed to stir at 25° C. for 2 hours. The reaction mixture was concentrated in vacuo, then purified using Flash column chromatography on silica gel (eluent:petroleum ether/ethyl acetate=5:1 to 1:2) to provide the product cap 33 (2 g, 86.9%). LC/MS: Anal. Calcd. For [M+H]⁺ C15H21BrN4O3; 385.08, 387.08. found 385.1, 387.1.

### Example 32

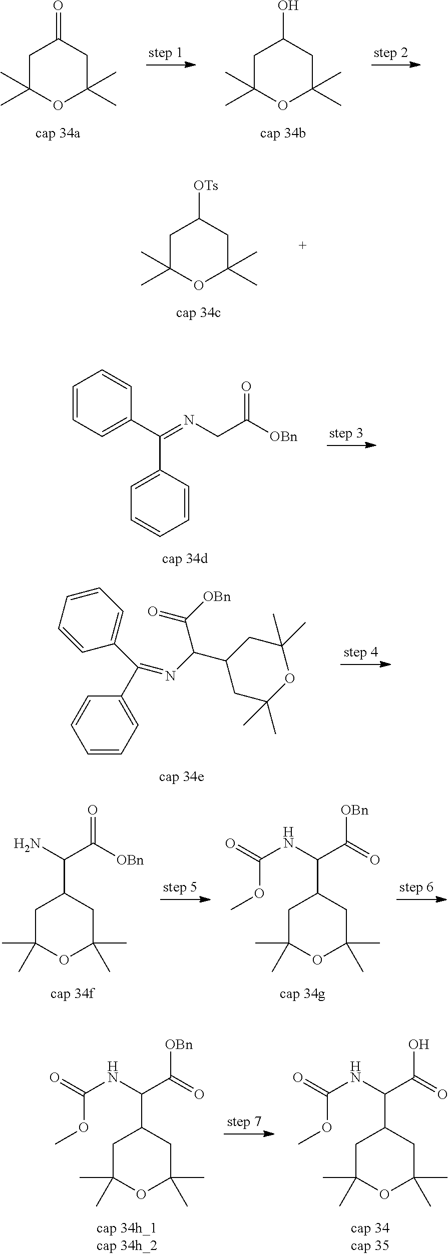

### Step 1

To a solution of cap 34a (0.2 g, 1.28 mmol) in EtOH (10 mL) was added NaBH₄ (0.073 g, 1.92 mmol) at 0° C. The reaction solution was allowed to stir at 25° C. for about 15 hours. The reaction was then quenched using water and extracted with EtOAc. The organic layer was then washed with brine, dried over Na₂SO₄ and concentrated in vacuo. The desired product cap 34b was obtained as a white solid and used directly for the next step (0.18 g, 89% yield). ¹H NMR (CDCl₃): δ 4.10 (s, 1H), 1.88-1.91 (m, 2H), 1.50-1.59 (m, 1H), 1.32-1.34 (m, 1H), 1.00-1.26 (m, 16H).

### Step 2

To a solution of cap 34b (0.16 g, 1.01 mmol) in DCM (10 mL) was added TsCl (0.387 g, 2.03 mmol) and DMAP (0.867 g, 4.05 mmol) at 0° C. Then the reaction mixture was allowed to stir at 25° C. for about 15 hours. Then the organic layer was washed with 1N HCl, brine, dried over Na₂SO₄ and concentrated in vacuo. The crude was purified using pre-TLC (Petroleum Ether/EtOAc=10/1) to provide cap 34c as a white solid (0.15 g, 47% yield). ¹H NMR (CDCl₃): δ 7.78-7.80 (d, J=8.0 Hz, 2H), 7.32-7.34 (d, J=8.0 Hz, 2H), 4.87-4.95 (m, 1H), 2.41 (s, 3H), 1.82-1.87 (m, 2H), 1.43-1.49 (m, 2H), 1.18 (s, 12H).

### Step 3

To a solution of cap 34c (0.15 g, 0.48 mmol) and cap 34d (0.158 g, 0.48 mmol) in toluene (5 mL) was added LiHMDS (0.58 mL, 0.58 mmol) under N₂. Then the reaction mixture was allowed to stir at 100° C. under microwave for 4 hours. The solution was washed with brine, dried over Na₂SO₄ and concentrated in vacuo. The crude cap 34e was used for the next step without purification (40 mg, 18% yield). LC/MS: Anal. Calcd. For [M+H]⁺ C31H33NO3; 470.26. found 470.1.

### Step 4

To a solution of cap 34e (40 mg crude) in THF was added HCl (aq) 5 mL. Then the mixture was allowed to stir at 20° C. for 2 hours. The solution was separated and the organic phase was discarded. The aqueous layer was extracted with EtOAc twice, and the organic layer was discarded too. Then the aqueous layer was then adjusted to 8 by adding NaHCO₃ (aq). Then the resulting solution was extracted with EtOAc three times. The EtOAc was removed in vacuo to provide cap 34f as colorless oil (10 mg, 38% yield). LC/MS: Anal. Calcd. For [M+H]⁺ C18H27NO3: 306.20. found 306.12.

### Step 5

To a solution of cap 34f (10 mg, 0.033 mmol) in DCM (10 mL) was added methyl carbonochloridate (5 mg, 0.049 mmol) and DIPEA (9 mg, 0.066 mmol). After stirring at 20° C. for 2 hours, the solution was washed with brine, dried over Na₂SO₄ and concentrated in vacuo to provide cap 34g (10 mg, 83% yield). LC/MS: Anal. Calcd. For [M+H]⁺ C20H29NO5; 364.20. found 364.1.

### Step 6

Cap 34h_1 and cap 34h_2 were separated by SFC from cap 34g (0.25 g, 0.69 mmol) using the following condition as a white solid:

Column: Chiralpak AS-H 250×4.6 mm I.D.

Mobile phase: ethanol (0.05% DEA) in CO₂ from 5% to 40%

Flow rate: 2.35 mL/min

Wavelength: 220 nm

Cap 34h_1 (80 mg, 64% yield). LC/MS: Anal. Calcd. For [M+H]⁺ C20H29NO5: 364.20. found 364.1.

Cap 34h_2 (70 mg, 56% yield). LC/MS: Anal. Calcd. For [M+H]⁺ C20H29NO5: 364.20. found 364.1.

### Step 7

A solution of cap 34h_1 (80 mg, 0.22 mmol) in 20 mL MeOH was added 10 mg Pd/C and stirred at 25° C. for 16 hours under H₂ (50 psi). The mixture was filtered and the filtrate was concentrated in vacuo to provide the compound cap 34 (61 mg, 100% yield). LC/MS: Anal. Calcd. For [M+H]⁺ C13H23NO5; 274.16. found: 274.2.

Cap 35 was prepared using the same method from cap 34h_2 (52 mg, 99% yield). LC/MS: Anal. Calcd. For [M+H]⁺ C13H23NO5; 274.16. found: 274.2.

### Example 33

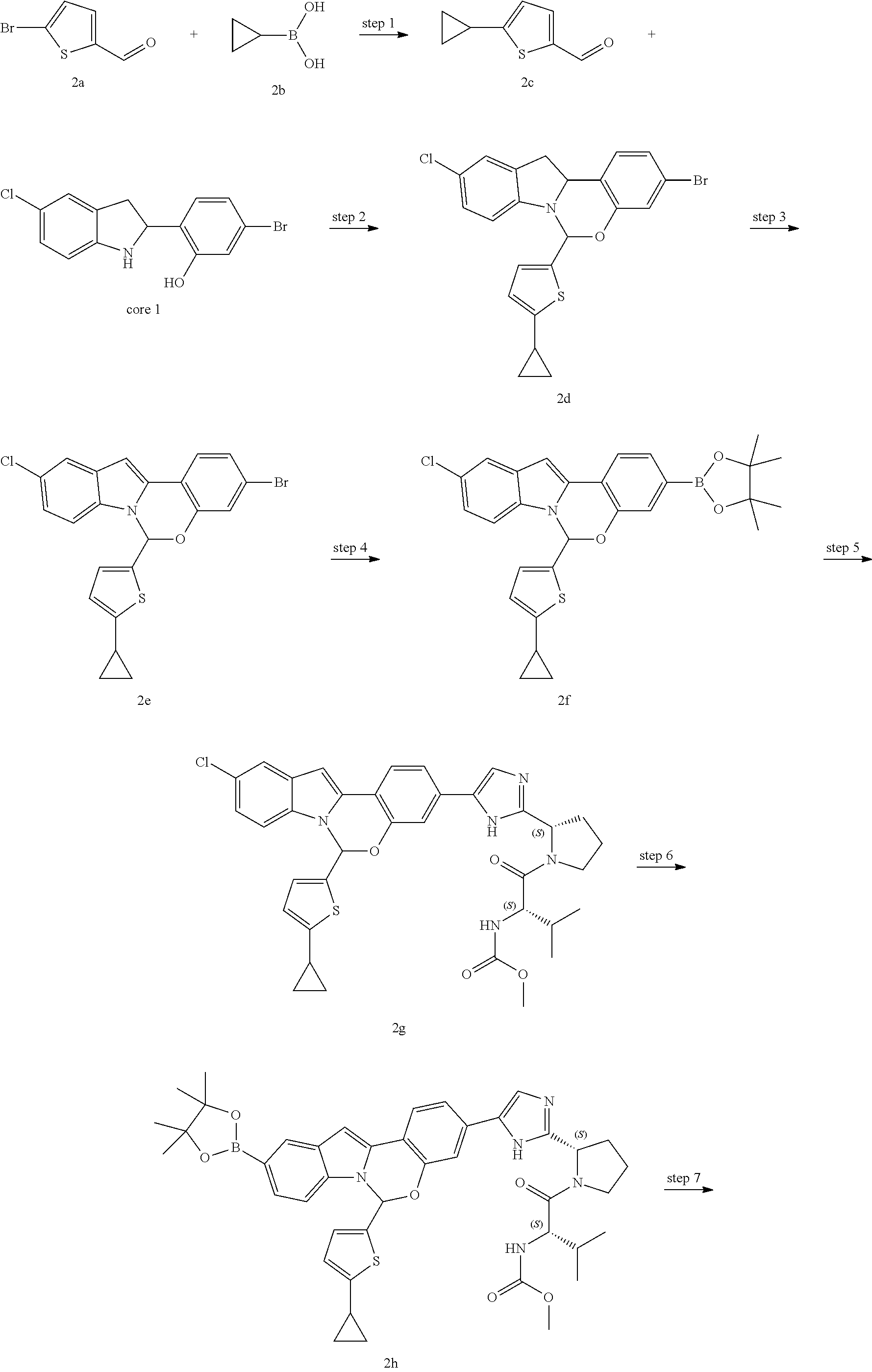

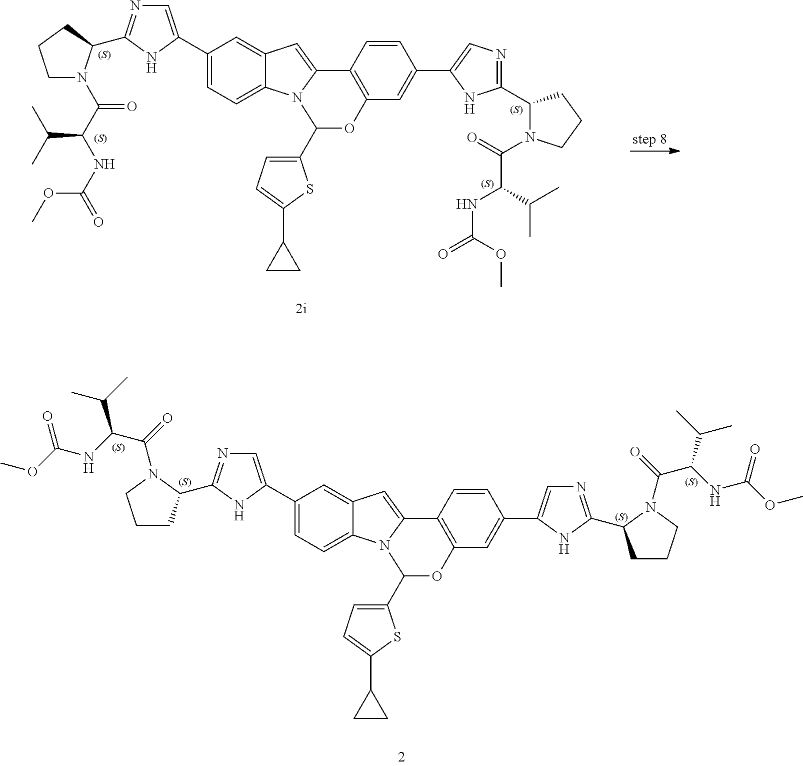

### Step 1

A solution of 2a (25 g, 130 mmol), 2b (34 g, 390 mmol), Pd(OAc)₂ (1.7 g, 7.6 mmol), cataCXium® A (4.6 g, 13 mmol) and Cs₂CO₃ (127 g, 390 mmol) in toluene/H₂O (660 mL, toluene/H₂O=10/1) was allowed to stir at 100° C. for 16 hours under N₂ atmosphere. The solution was extracted with EtOAc. The combined organic extracts were dried over Na₂SO₄, purified using Flash column chromatography on silica gel, eluting with petroleum ether:ethyl acetate (50:1) to provide 2c (18.5 g, 95% yield). ¹H NMR (CDCl₃) δ: 9.78 (s, 1H), 7.58 (d, J=3.91 Hz, 1H), 6.86 (d, J=3.52 Hz, 1H), 2.12-2.22 (m, 1H), 1.10-1.19 (m, 2H), 0.78-0.90 (m, 2H).

### Step 2

To a mixture of 2c (3.5 g, 23.1 mmol) and core 1 (5 g, 15.4 mmol) in anhydrous CH₃CN (40 mL) was added TFA (1 mL). The mixture was allowed to stir at 25° C. for 6 hours. The reaction mixture became a clear solution and then a solid appeared. The solid was collected by filtration and washed with CH₃CN to provide 2d (5.5 g, 78% yield).

### Step 3

To a solution of 2d (5.5 g, 12 mmol) in dry toluene (50 mL) was added DDQ (4.1 g, 18 mmol). After refluxing for 2 hours, the solvent was removed in vacuo and diluted with EtOAc. The organic layer was washed with saturated Na₂S₂O₃ aqueous and brine, dried over Na₂SO₄, filtered and concentrated in vacuo. The residue obtained was washed with MeOH (20 mL), filtered and the collected solid was the product 2e (4.4 g, 82% yield).

### Step 4

A suspension of 2e (4.3 g, 9.43 mmol), bis(pinacolato)diboron (2.9 g, 11.32 mmol), KOAc (2.3 g, 23.58 mmol) and Pd(dppf)Cl₂ (345 mg, 0.5 mmol) in dioxane (60 mL) was allowed to stir at 100° C. for 2 hours under N₂ atmosphere. The reaction mixture was cooled and concentrated in vacuo, the residue obtained was purified using Flash column chromatography on silica gel, eluting with petroleum ether:ethyl acetate (30:1) to provide 2f (4 g, 84% yield). LC/MS: Anal. Calcd. For [M+H]⁺ C28H27BClNO3S; 504.15. found 504.3.

### Step 5

A suspension of 2f (4 g, 7.94 mmol), cap 31 (3.3 g, 8.73 mmol), Na₂CO₃ (2.1 g, 19.85 mmol) and Pd(dppf)Cl₂ (292 mg, 0.4 mmol) in THF/H₂O (v/v=5/1, 100 mL) was allowed to stir at 80° C. for about 15 hours under N₂ atmosphere. After that, the mixture was washed with water and extracted with ethyl acetate, washed with brine and dried over anhydrous sodium sulfate. After being concentrated in vacuo, the residue obtained was purified using Flash column chromatography on silica gel, eluting with petroleum ether:ethyl acetate (3:1 to 1:1) to provide 2g (4 g, 75% yield). LC/MS: Anal. Calcd. For [M+H]⁺ C36H36ClN5O4S: 670.22. found 670.3.

### Step 6

To a mixture of 2g (5.1 g, 7.61 mmol), bis(pinacolato)diboron (2.3 g, 9.13 mmol), KOAc (1.9 g, 19.03 mmol), Pd₂(dba)₃ (551 mg, 0.53 mmol), X-Phos (508 mg 1.06 mmol) degassed and sealed under N₂ was added dry dioxane. The mixture was allowed to stir at 100° C. for about 15 hours. After that, cooling to 25° C., concentrated in vacuo and the residue obtained was purified using Flash column chromatography on silica gel, eluting with petroleum ether:ethyl acetate (3:1 to 1:1) to provide 2 h (4.8 g, 83% yield). LC/MS: Anal. Calcd. For [M+H]⁺ C42H48BN5O6S: 762.34. found 762.2.

### Step 7

A mixture of 2 h (2.4 g, 3.15 mol), cap 31 (1.3 g, 3.46 mmol), Na₂CO₃ (0.83 g, 7.88 mol) and Pd(dppf)Cl₂ (162 mg, 0.22 mmol) in THF/H₂O (v/v=5/1, 50 mL) was allowed to stir at 80° C. under N₂ atmosphere for about 15 hours. After that, the mixture was washed with water and extracted with ethyl acetate, washed with brine and dried over anhydrous sodium sulfate. After being concentrated in vacuo, the residue obtained was purified using Flash column chromatography on silica gel, eluting with petroleum ether:ethyl acetate (3:1 to 1:2) to provide 2i (1.8 g, 62% yield). LC/MS: Anal. Calcd. For [M+H]⁺ C50H57N9O7S; 928.41. found 928.7.

### Step 8

Compound 2 was got from compound 2i (1.8 g) by SFC using the following conditions:

Instrument: Thar SFC

Column: OD-3, 150×4.6 mm, 5 um

Mobile phase: A for CO₂ and B for MeOH (0.05% DEA)

Gradient: B 5% to 40 for A

Flow rate: 2.5 mL/min

Back pressure: 100 bar

Column temperature: 35° C.

Wavelength: 230 nm

Compound 2 (530 mg, 30% yield). ¹H NMR (MeOD) δ: 7.98 (s, 1H), 7.92-7.94 (d, J=7.6 Hz, 1H), 7.88 (s, 1H), 7.73 (s, 1H), 7.71 (s, 1H), 7.41-7.50 (m, 4H), 7.15 (s, 1H), 6.45-6.48 (m, 2H), 5.18-5.25 (m, 2H), 4.08-4.22 (m, 4H), 3.83-3.89 (m, 2H), 3.63 (s, 6H), 2.55 (m, 2H), 1.88-2.26 (m, 9H), 0.86-0.93 (m, 14H), 0.49-0.53 (m, 2H). LC/MS: Anal. Calcd. For [M+H]⁺ C50H57N9O7S; 928.41. found 928.3.

The following compounds of the present invention were made using the methods described in the Example above and substituting the appropriate reactants or reagents.

| | | | Ob- |
|---|---|---|---|
| | | | served |
| | | | [M + |
| ID | Structure | Isomer | H]+ |
| 504 | 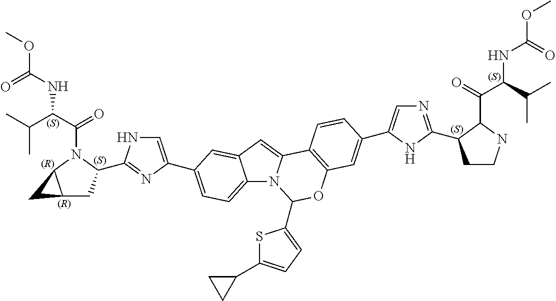 | racemic | 940.74 |
| 523 524 525 | 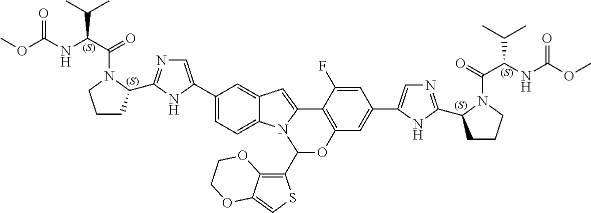 | Isomer 1 Isomer 2 racemic | 964.95 964.95 964.89 |
| 526 527 | 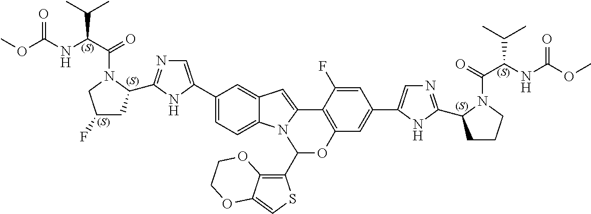 | Isomer 1 Racemic | 984.00 982.90 |
| 607 | 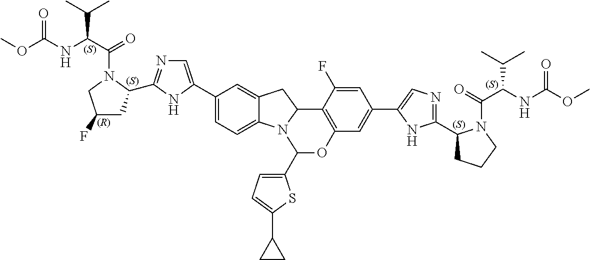 | racemic | 947.11 |
| 608 609 610 | 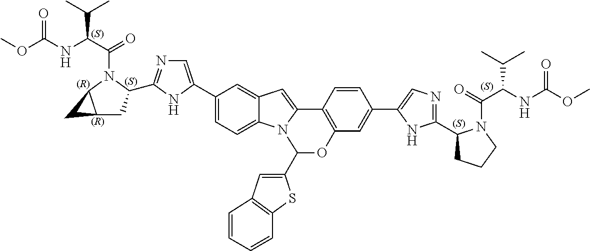 | Isomer 2 Isomer 1 racemic | 950.50 950.40 950.70 |
| 617 618 619 | 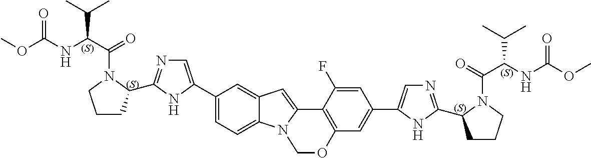 | Isomer 1 Isomer 2 racemic | 975.23 975.26 975.17 |
| 623 624 625 | 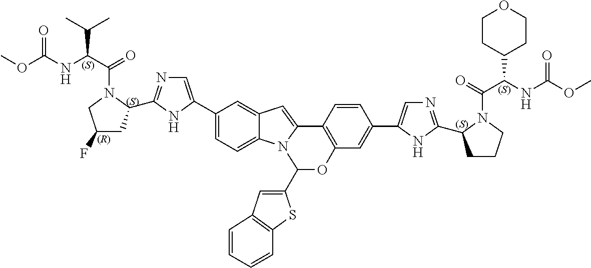 | racemic Isomer 1 Isomer 2 | 998.93 998.97 998.93 |

### Example 34

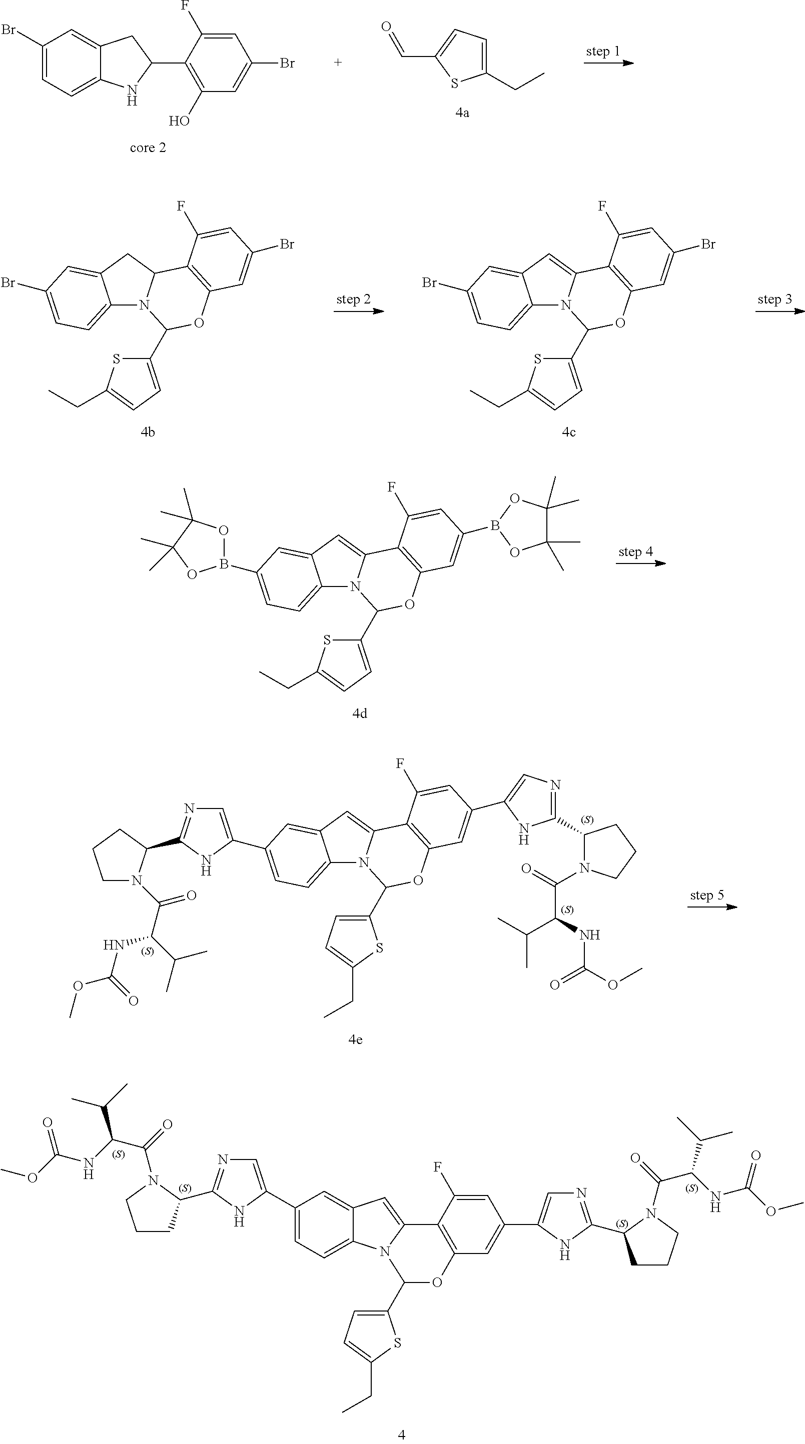

### Step 1

To a mixture of core 2 (20 g, 51.7 mmol) and 4a (21.7 g, 155.0 mmol) in anhydrous CH₃CN (150 mL) was added TFA (2 mL). The mixture was allowed to stir at 25° C. for 6 hours. The reaction mixture became a clear solution and then a solid appeared. The solid was collected by filtration and washed with CH₃CN to provide 4b (22.3 g, 85% yield). ¹H NMR (CDCl₃) δ: 7.26 (s, 1H), 7.23 (s, 1H), 6.75-6.89 (m, 5H), 6.63 (d, J=3.2 Hz, 1H), 5.13 (d, J=9.2 Hz, 1H), 3.57 (dd, J=16.4, 9.2 Hz, 1H), 3.27 (d, J=16.8 Hz, 1H), 2.80 (q, J=7.2 Hz, 2H), 1.29 (t, J=7.6 Hz, 3H).

### Step 2

The solution of 4b (22.3 g, 43.8 mmol) in dry toluene (200 mL) was added DDQ (14.9 g, 65.7 mmol). After refluxing for 2 hours, the solvent was removed in vacuo and diluted with EtOAc. The organic layer was washed with saturated Na₂S₂O₃ aqueous and brine, dried over Na₂SO₄, filtered and concentrated in vacuo. The residue obtained was washed with MeOH (50 mL), filtered and the solid was 4c (18.6 g, 84% yield).

### Step 3

A suspension of 4c (18.7 g, 36.9 mmol), bis(pinacolato)diboron (20.6 g, 81.1 mmol), KOAc (18.1 g, 184.4 mmol) and Pd(dppf)Cl₂ (2.7 g, 3.7 mmol) in dioxane (300 mL) was allowed to stir at 100° C. for 2 hours under N₂ atmosphere. The reaction mixture was cooled and concentrated in vacuo, the residue obtained was purified using Flash column chromatography on silica gel, eluting with petroleum ether:ethyl acetate (30:1) to provide 4d (19 g, 85% yield). LC/MS: Anal. Calcd. For [M+H]⁺ C33H38B2FNO5S; 602.26. found 602.4.

### Step 4

A suspension of 4d (10 g, 16.6 mmol), cap 31 (13.6 g, 36.6 mmol), Na₂CO₃ (8.8 g, 83.2 mmol) and Pd(dppf)Cl₂ (1.2 g, 1.67 mmol) in THF/H₂O (v/v=5/1, 250 mL) was allowed to stir at 80° C. for about 15 hours under N₂ atmosphere. After that, the mixture was washed with water and extracted with ethyl acetate, washed with brine and dried over anhydrous sodium sulfate. After being concentrated in vacuo, the residue obtained was purified using Flash column chromatography on silica gel, eluting with petroleum ether:ethyl acetate (3:1 to 1:2) to provide 4e (8 g, 51% yield). LC/MS: Anal. Calcd. For [M+H]⁺ C49H56FN9O7S; 935.41. found 934.5.

### Step 5

Compound 4 was obtained via purification of compound 43e using SFC under the following conditions:

Instrument: Thar SFC

Column: AS-H, 150×4.6 mm, 5 um

Mobile phase: A for CO₂ and B for IPA (0.05% DEA)

Gradient: B 5% to 40 for A

Flow rate: 2.5 mL/min

Back pressure: 100 bar

Column temperature: 35° C.

Wavelength: 230 nm

Compound 4 (6 g, 29% yield). ¹H NMR (MeOD) δ: 8.01 (s, 1H), 7.95 (s, 1H), 7.81 (s, 1H), 7.75 (s, 1H), 7.51-7.57 (m, 1H), 7.46-7.51 (m, 1H), 7.39 (d, J=10.8 Hz, 1H), 7.30 (s, 1H), 7.17 (d, J=2.4 Hz, 1H), 6.47-6.56 (m, 2H), 5.22 (dt, J=15.2, 7.2 Hz, 2H), 4.21 (t, J=7.2 Hz, 2H), 4.08 (m, 2H), 3.83-3.94 (m, 2H), 3.64 (s, 6H), 2.67 (q, J=7.2 Hz, 2H), 2.54 (d, J=5.6 Hz, 2H), 1.99-2.32 (m, 8H), 1.13 (t, J=7.2 Hz, 3H), 0.85-1.00 (m, 12H). LC/MS: Anal. Calcd. For [M+H]⁺ C49H56FN9O7S; 935.41. found 935.3.

The following compounds of the present invention were made using the methods described in the Example above and substituting the appropriate reactants or reagents.

| | | | Ob- |
|---|---|---|---|
| | | | served |
| | | | [M + |
| ID | Structure | Isomer | H]+ |
| 1 703 704 | 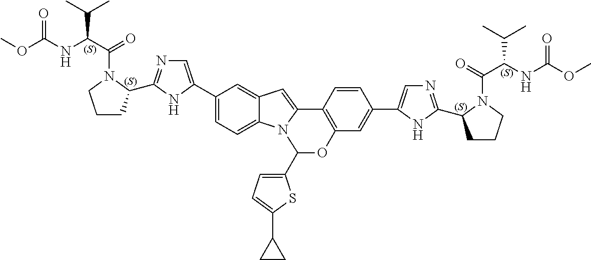 | Isomer 1 racemic Isomer 1 | 928.1 928.1 929.5 |
| 3 | 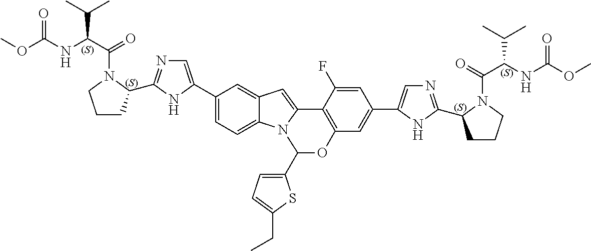 | Isomer 1 | 934.8 |
| 5 | 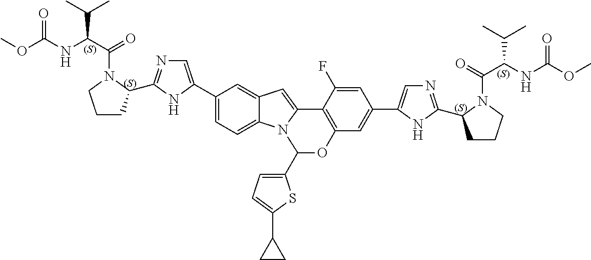 | Isomer 1 | 946.5 |
| 13 14 | 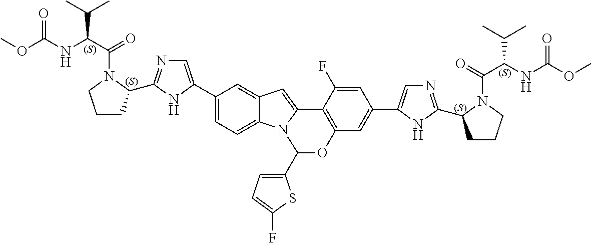 | Isomer 1 Isomer 2 | 924.4 924.6 |
| 15 16 | 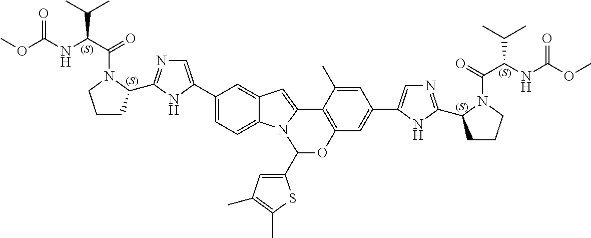 | Isomer 1 Isomer 2 | 930.6 930.6 |
| 17 18 | 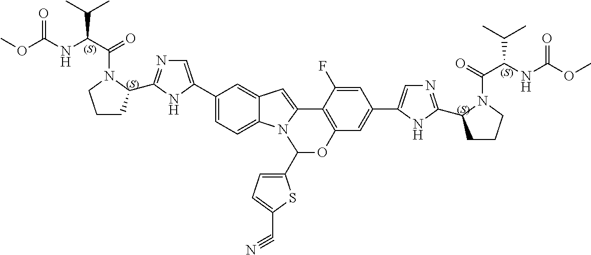 | Isomer 1 Isomer 2 | 931.4 931.6 |
| 19 20 | 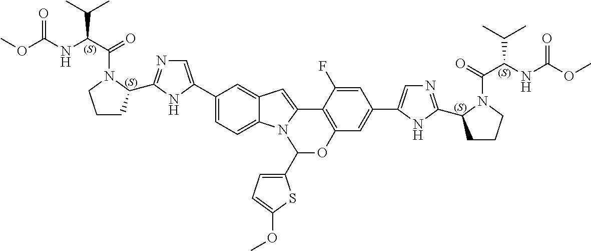 | Isomer 1 Isomer 2 | 936.3 936.3 |
| 21 22 | 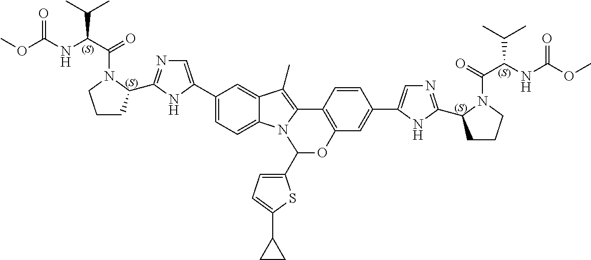 | Isomer 1 Isomer 2 | 942.6 942.6 |
| 23 24 | 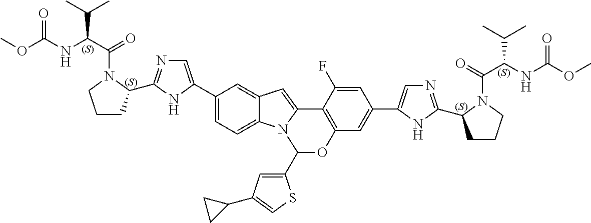 | Isomer 1 Isomer 2 | 946.4 946.6 |
| 25 26 | 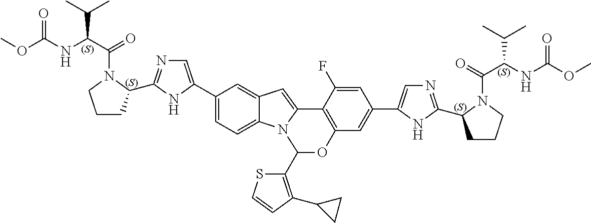 | Isomer 1 Isomer 2 | 947.0 947.0 |
| 27 | 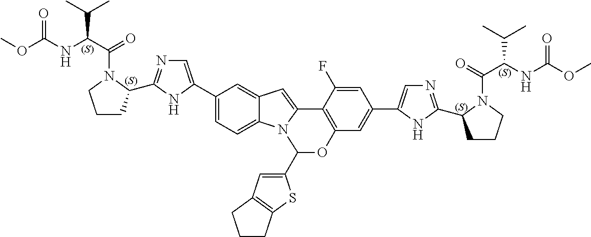 | Isomer 1 | 946.8 |
| 33 | 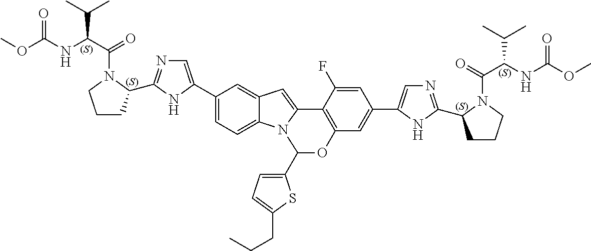 | Isomer 1 | 948.8 |
| 35 | 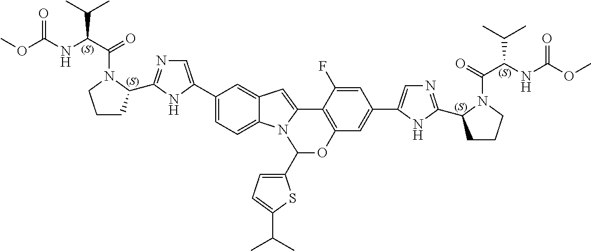 | Isomer 1 | 948.6 |
| 37 38 | 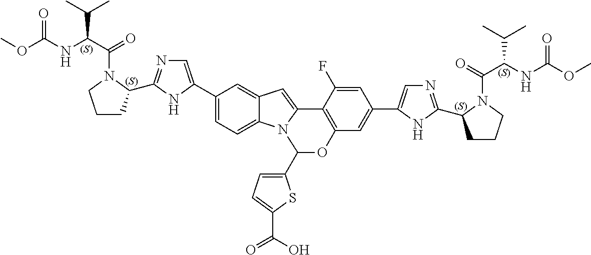 | Isomer 1 Isomer 2 | 950.4 950.4 |
| 43 44 | 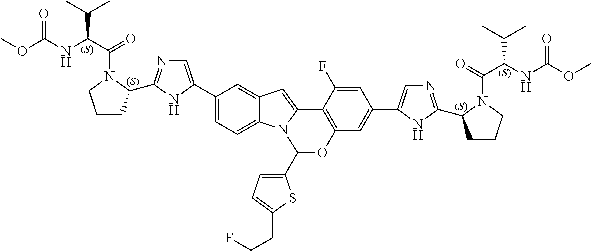 | Isomer 1 Isomer 2 | 952.4 952.4 |
| 46 | 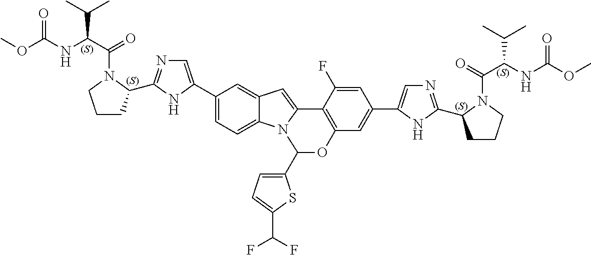 | Isomer 2 | 956.4 |
| 47 48 | 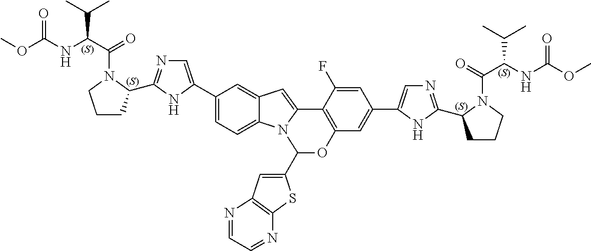 | Isomer 1 Isomer 2 | 958.6 958.6 |
| 50 | 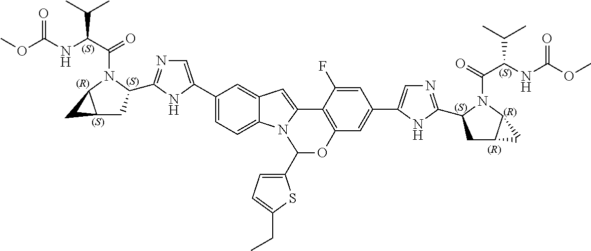 | Isomer 2 | 958.5 |
| 51 52 | 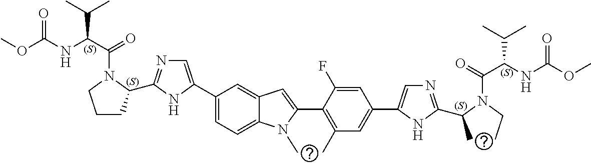 | Isomer 1 Isomer 2 | 960.6 960.5 |
| 53 | 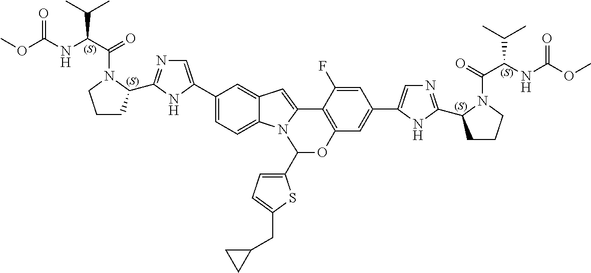 | Isomer 1 | 960.5 |
| 55 | 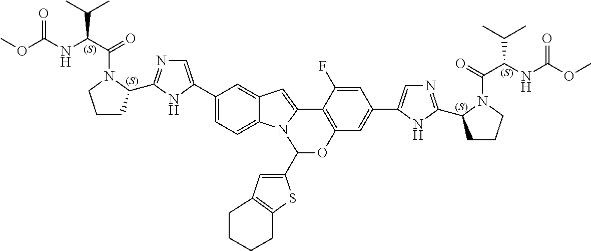 | Isomer 1 | 960.6 |
| 57 | 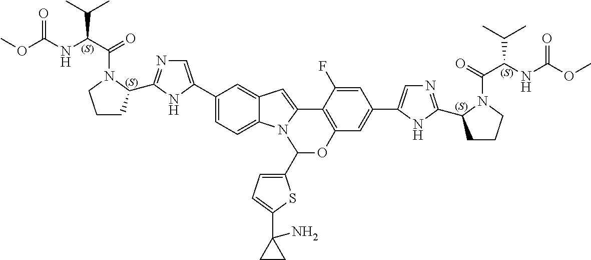 | racemic | 961.6 |
| 60 | 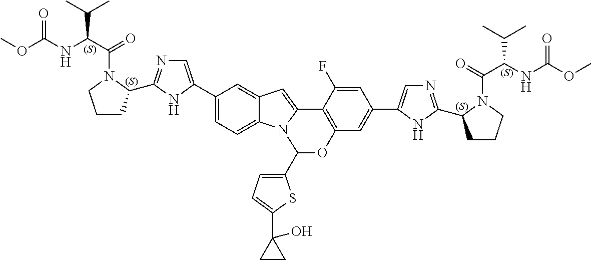 | Isomer 1 | 962.4 |
| 62 | 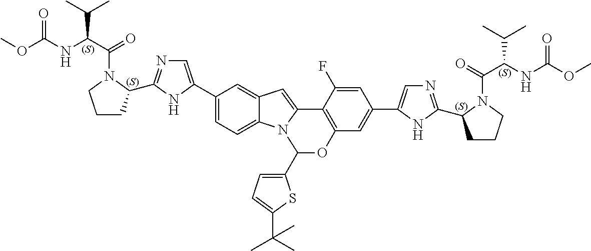 | Isomer 1 | 962.6 |
| 64 | 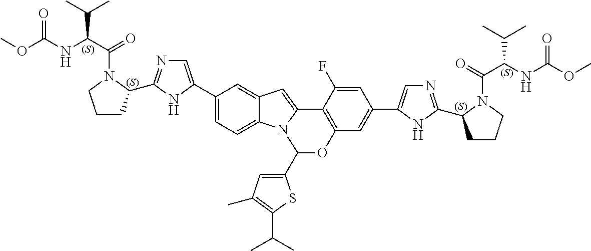 | racemic | 962.6 |
| 65 66 | 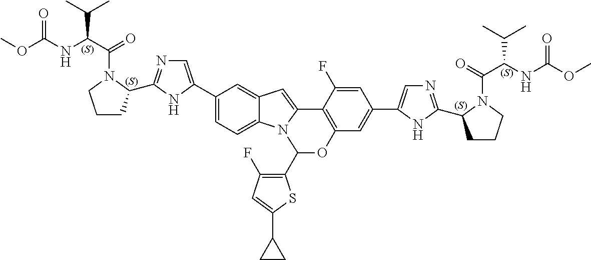 | Isomer 1 Isomer 2 | 964.6 964.6 |
| 71 72 | 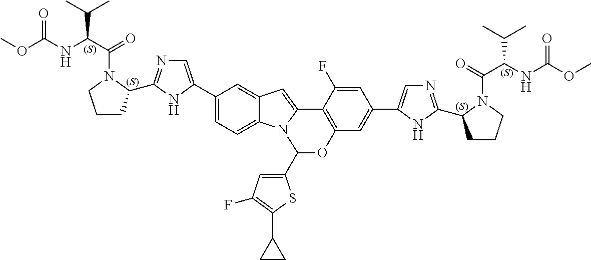 | Isomer 1 Isomer 2 | 964.6 964.4 |
| 73 | 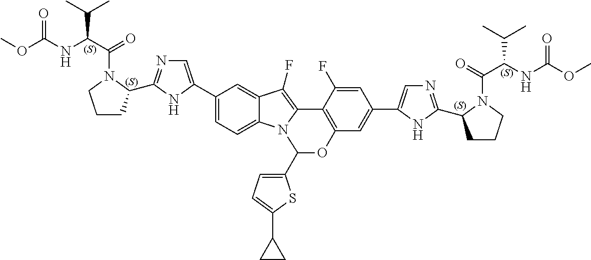 | Isomer 1 | 964.8 |
| 81 | 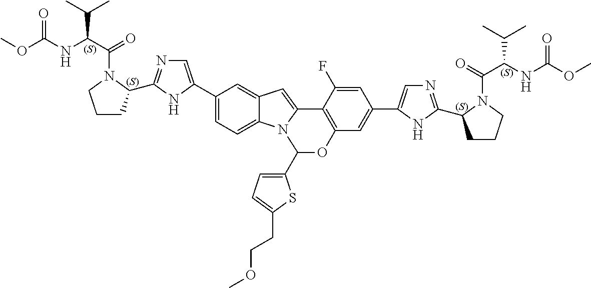 | Isomer 1 | 964.6 |
| 83 84 | 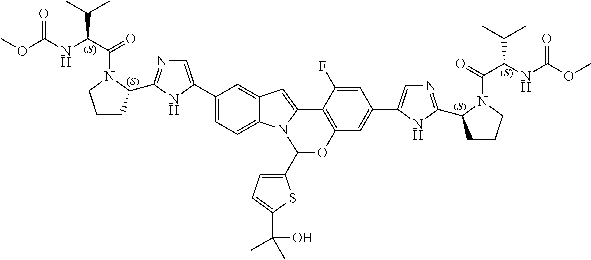 | Isomer 1 Isomer 2 | 964.6 964.6 |
| 85 86 | 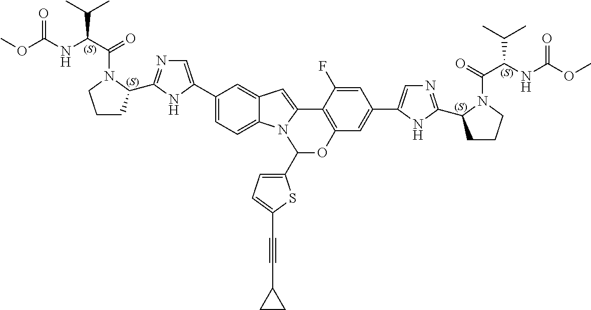 | Isomer 1 Isomer 2 | 970.4 970.6 |
| 87 88 | 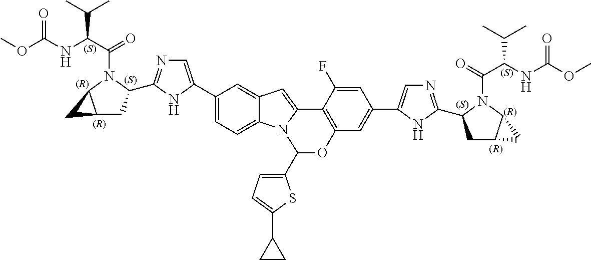 | Isomer 1 Isomer 2 | 970.8 970.4 |
| 91 | 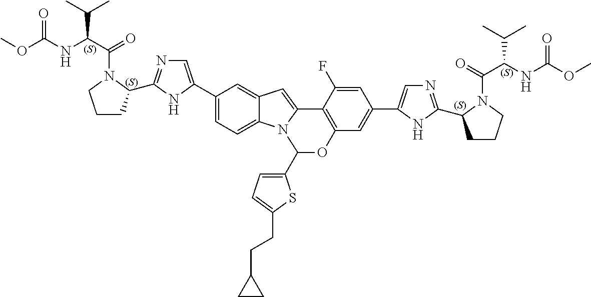 | Isomer 1 | 974.5 |
| 103 104 | 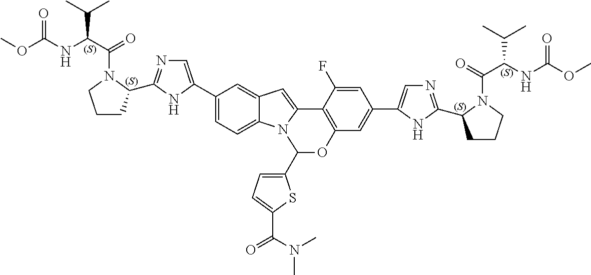 | Isomer 1 Isomer 2 | 977.6 977.6 |
| 110 111 | 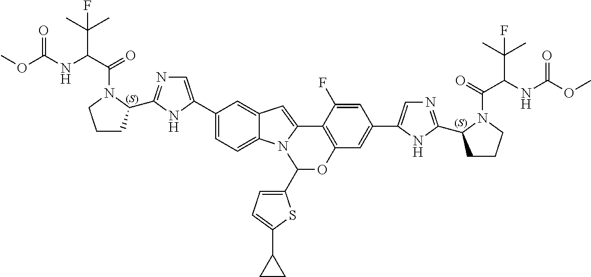 | Isomer 1 Isomer 2 | 982.6 982.4 |
| 116 117 118 119 | 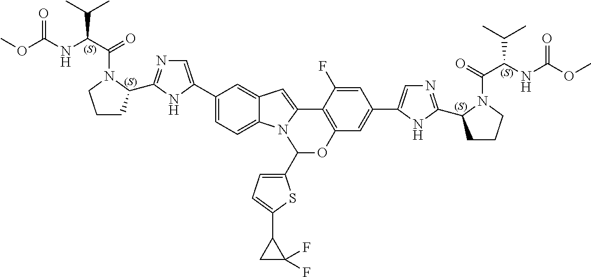 | Isomer 1 Isomer 2 Isomer 3 Isomer 4 | 982.6 982.6 982.6 982.6 |
| 120 121 | 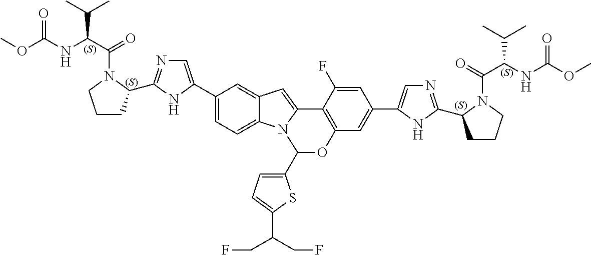 | Isomer 1 Isomer 2 | 984.4 984.6 |
| 124 | 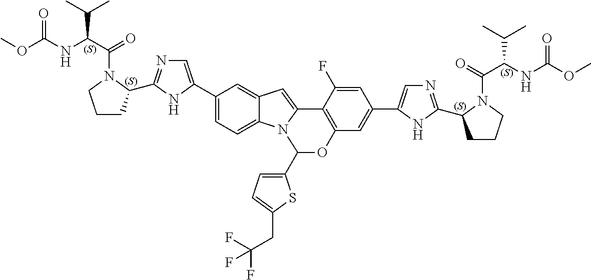 | Isomer 1 | 988.4 |
| 132 133 | 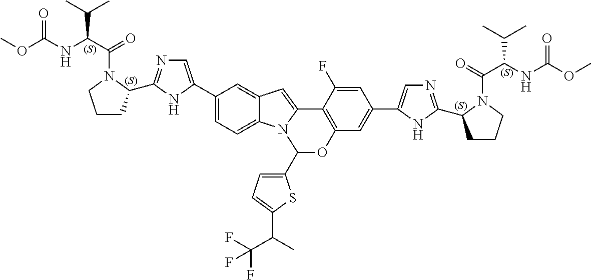 | Isomer 1 Isomer 2 | 1002.6 1002.6 |
| 262 263 | 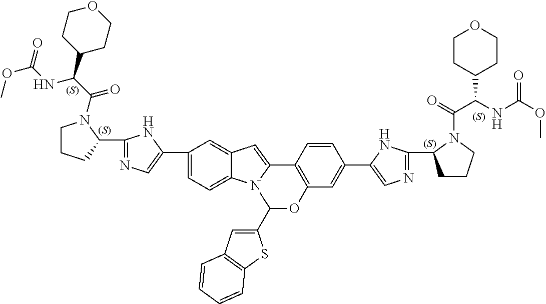 | Isomer 1 Isomer 2 | 1022.4 1022.6 |
| 264 | 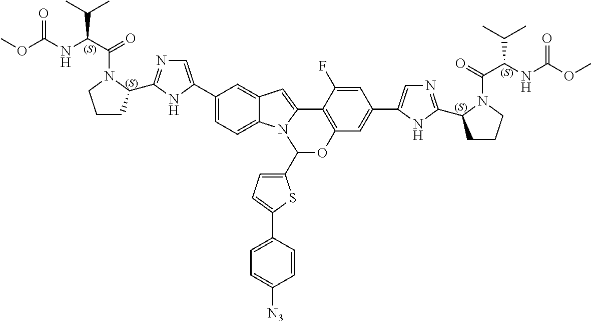 | racemic | 1023.8 |
| 279 280 | 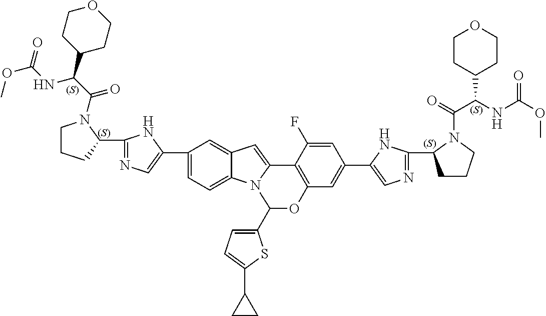 | Isomer 1 Isomer 2 | 1030.8 1030.5 |
| 291 292 293 294 295 296 297 298 | 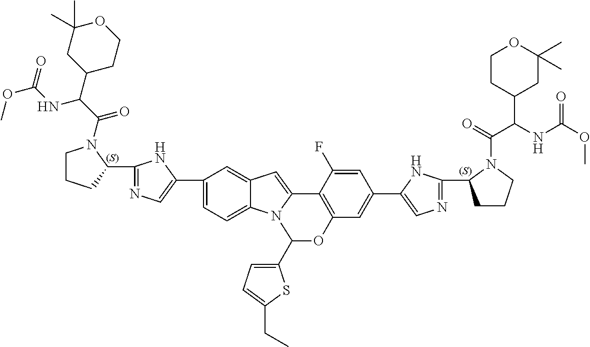 | Isomer 1 Isomer 2 Isomer 3 Isomer 4 Isomer 5 Isomer 6 Isomer 7 Isomer 8 | 1074.6 1074.6 1074.8 1074.8 1074.6 1074.6 1074.6 1074.2 |
| 299 300 | 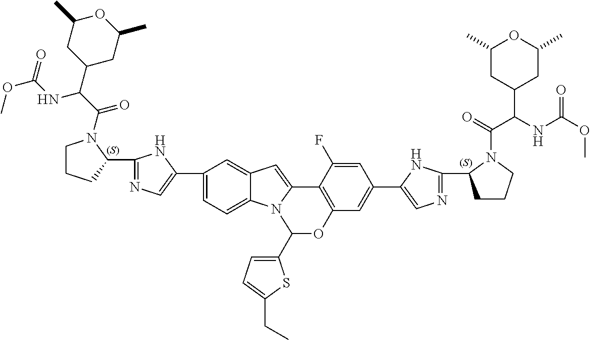 | Isomer 1 Isomer 2 | 1074.2 1074.6 |
| 301 302 304 | 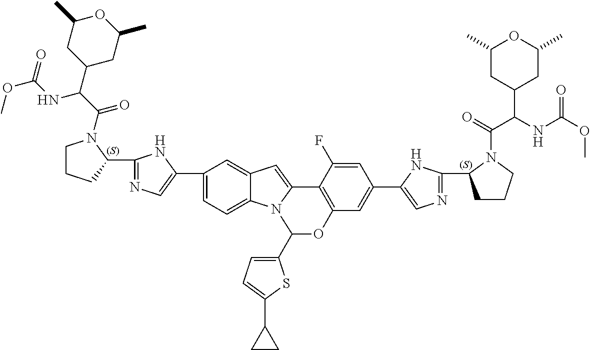 | Isomer 1 Isomer 2 Isomer 4 | 1086.7 1086.7 1086.9 |
| 305 306 307 308 309 310 | 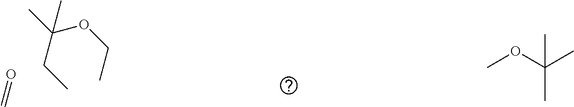 | Isomer 1 Isomer 2 Isomer 3 Isomer 4 Isomer 5 Isomer 6 | 1086.6 1086.6 1086.6 1086.8 1086.8 1086.8 |
| 311 312 | 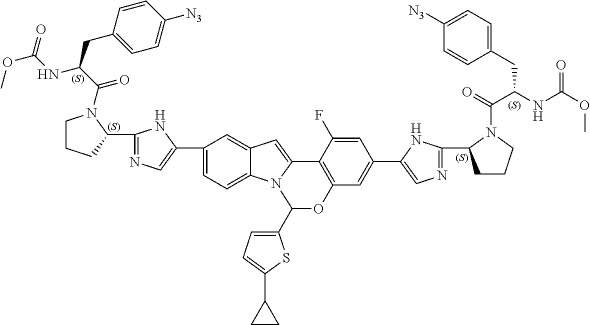 | Isomer 1 Isomer 2 | 1106.4 1106.4 |
| 313 | 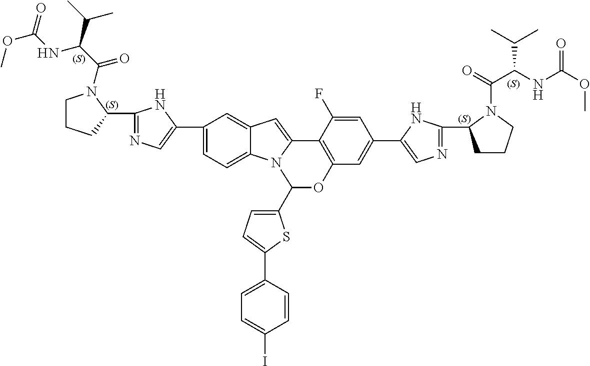 | Racemic | 1108.4 |
| 315 316 | 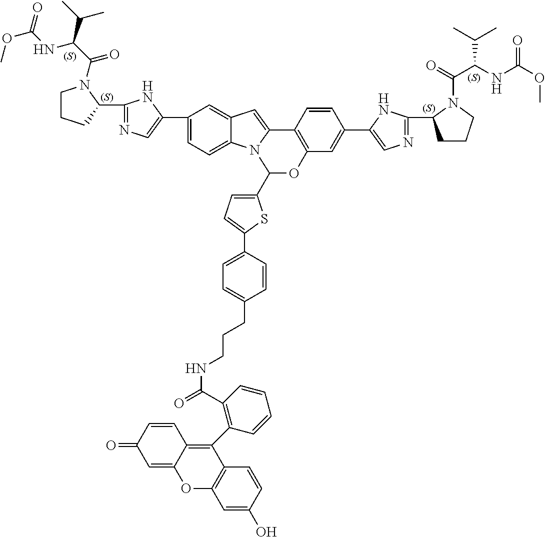 | Isomer 1 Isomer 2 | 1135.8 1135.8 |
| 317 318 | 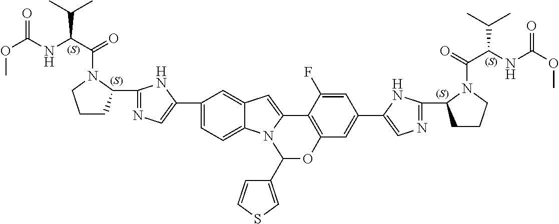 | Isomer 1 Isomer 2 | 906.6 906.6 |
| 319 320 | 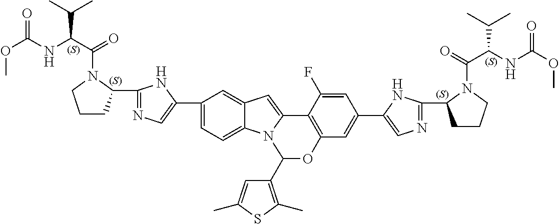 | Isomer 1 Isomer 2 | 934.4 934.4 |
| 321 322 | 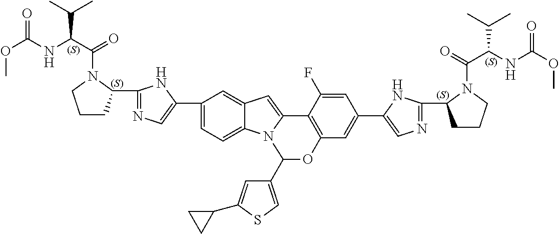 | Isomer 1 Isomer 2 | 946.6 946.6 |
| 323 324 | 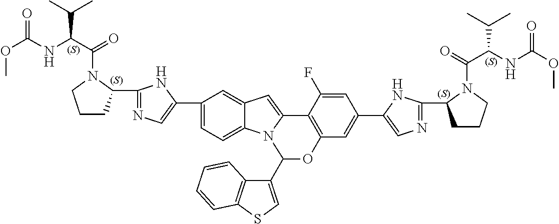 | Isomer 1 Isomer 2 | 956.6 956.6 |
| 326 327 | 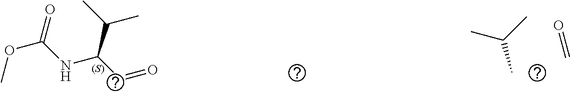 | Isomer 1 Isomer 2 | 930.4 930.4 |
| 328 329 | 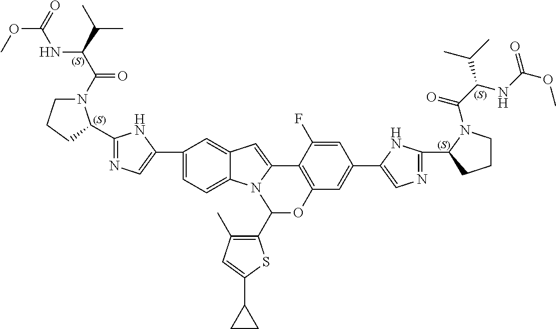 | Isomer 1 Isomer 2 | 960.4 960.4 |
| 330 331 | 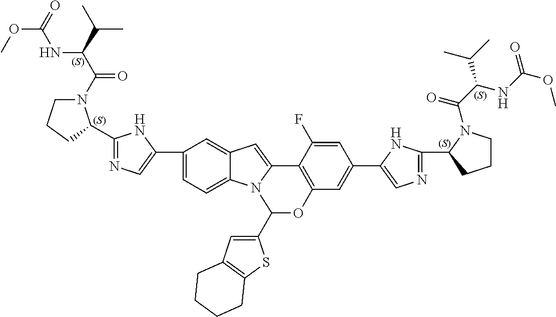 | Isomer 1 Isomer 2 | 962.4 962.4 |
| 332 333 | 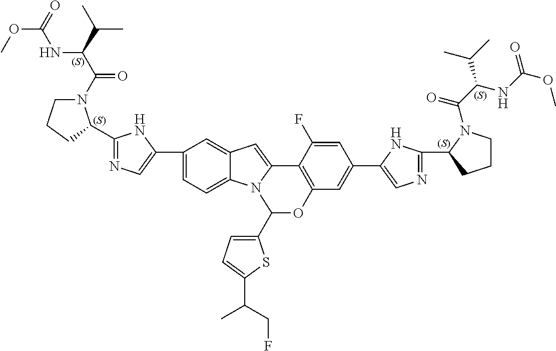 | Isomer 1 Isomer 2 | 966.4 966.4 |
| 383 384 385 | 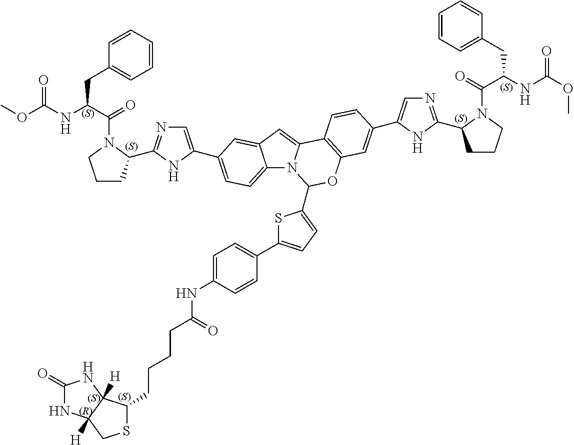 | racemic Isomer 1 Isomer 2 | 1301.5 1301.5 1301.5 |
| 386 387 388 | 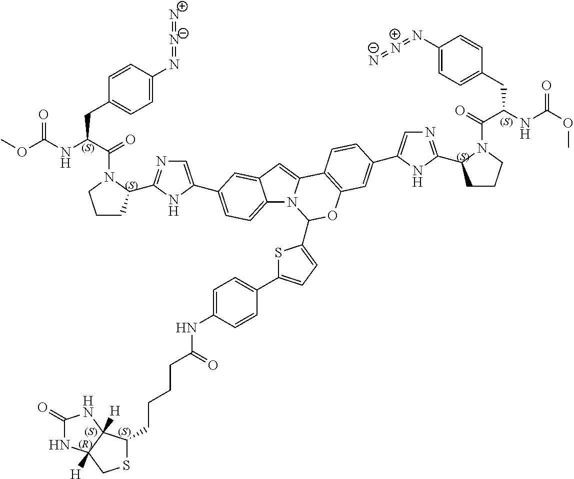 | racemic Isomer 1 Isomer 2 | 1383.5 1383.5 1383.5 |
| 391 392 | 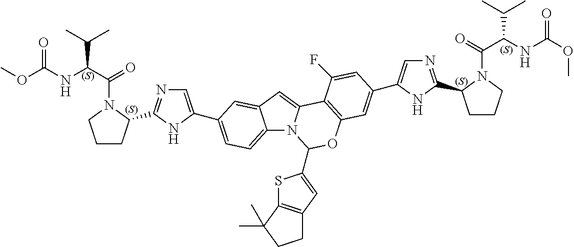 | Isomer 1 Isomer 2 | 974.1 974.1 |
| 393 394 395 396 | 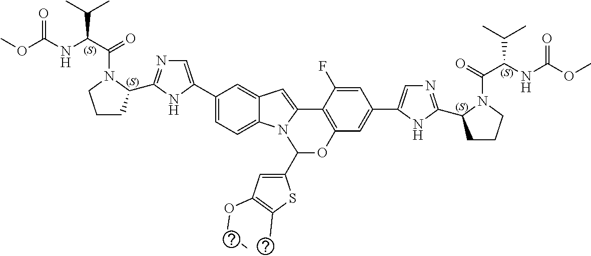 | Isomer 1 Isomer 2 Isomer 3 Isomer 4 | 976.1 976.1 976.1 976.1 |
| 397 398 | 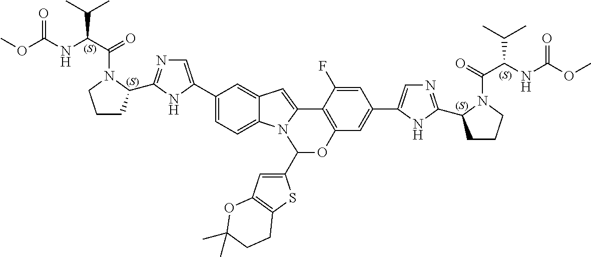 | Isomer 1 Isomer 2 | 990.1 990.1 |
| 399 400 | 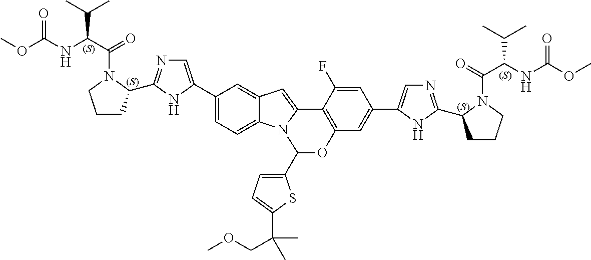 | Isomer 1 Isomer 2 | 992.1 992.1 |
| 600 | 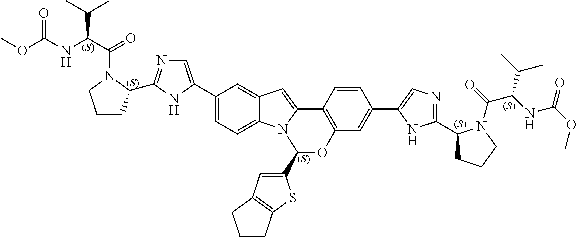 | Isomer 1 | 928.91 |
| 758 | 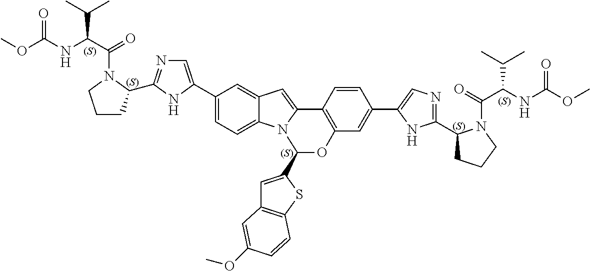 | | 969.77 |
| 805 806 | 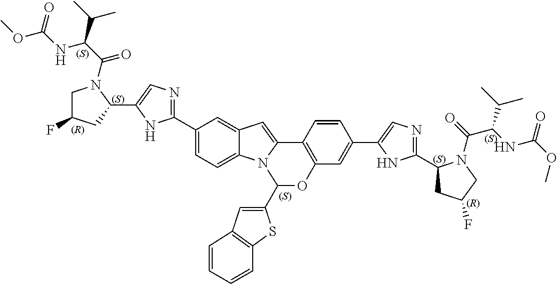 | isomer 2 isomer 1 | 974.90 974.90 |
| 809 810 811 | 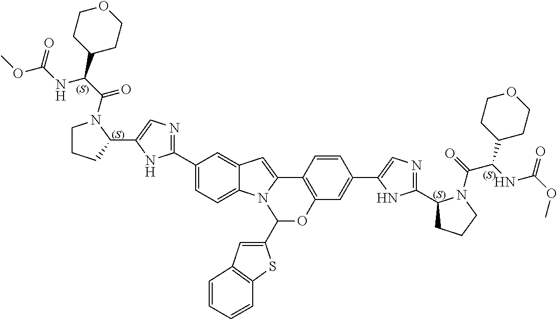 | isomer 1 racemic isomer 2 | 1023.40 1023.40 1023.40 |
| 814 | 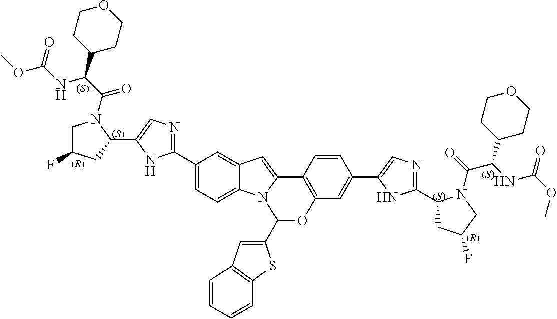 | racemic | 1059.40 |

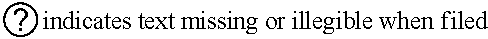

### Example 35

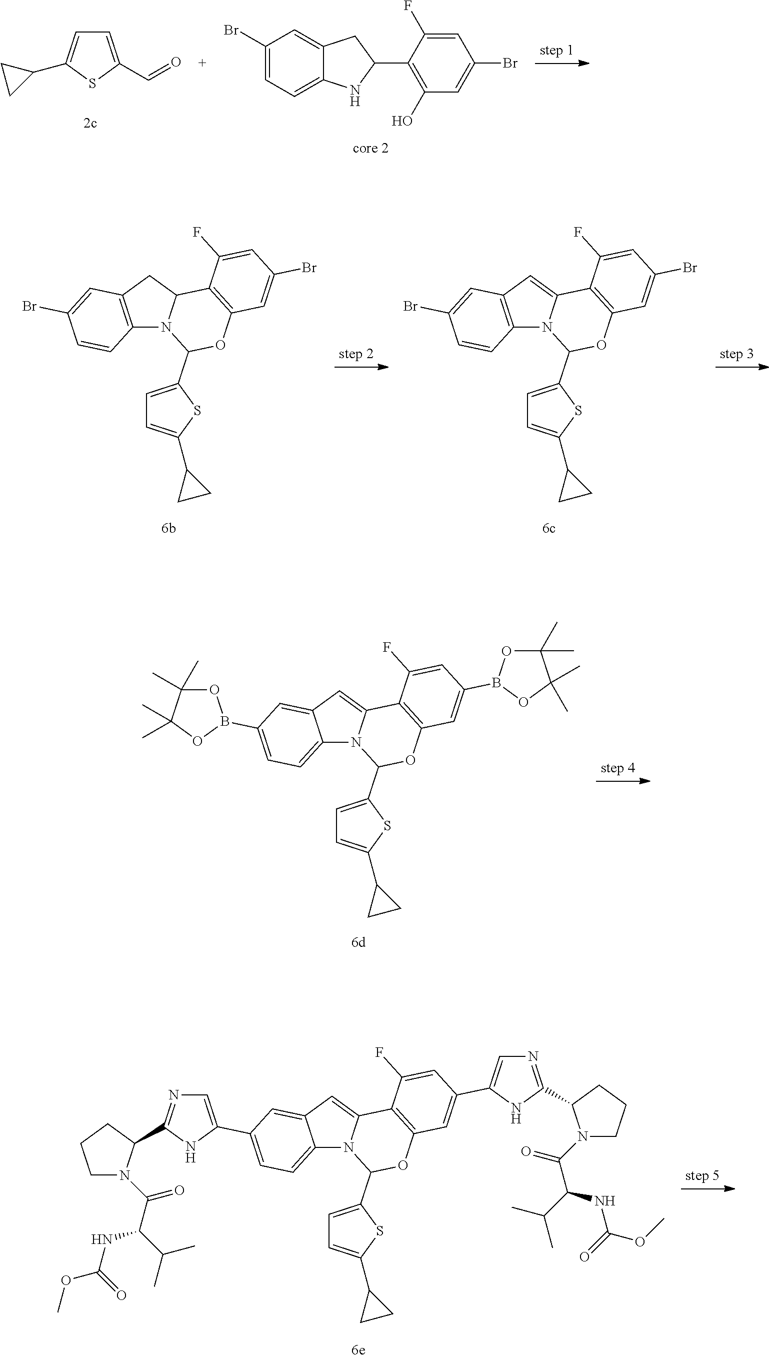

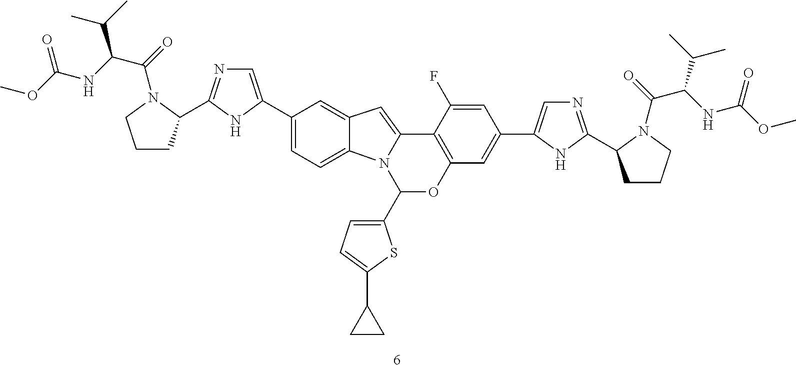

### Step 1

Compound 2c was made from Example 33. To a mixture of 2c (15.7 g, 103.4 mmol) and core 2 (20 g, 51.7 mmol) in anhydrous CH₃CN (200 mL) was added TFA (2 mL). The mixture was allowed to stir at 25° C. for 6 hours. The reaction mixture became a clear solution and then a solid appeared. The solid was collected by filtration and washed with CH₃CN to provide compound 6b (23.1 g, 85% yield). ¹H NMR (CDCl₃) δ: 7.28 (s, 1H), 7.23 (s, 1H), 6.76-6.86 (m, 5H), 6.60 (s, 1H), 5.15 (d, J=9.2 Hz, 1H), 3.54-3.61 (m, 1H), 3.27 (d, J=8.4 Hz, 1H), 1.99-2.05 (m, 1H), 0.96-1.01 (m, 2H), 0.69-0.73 (m, 2H).

### Step 2

To a solution of 6b (23.1 g, 44.3 mmol) in dry toluene (200 mL) was added DDQ (15.1 g, 66.5 mmol). After refluxing for 2 hours, the solvent was removed in vacuo and diluted with EtOAc. The organic layer was washed with saturated Na₂S₂O₃ aqueous and brine, dried over Na₂SO₄, filtered and concentrated in vacuo. The residue obtained was washed with MeOH (50 mL), filtered and the solid just was 6c (20 g, 86% yield). ¹H NMR (CDCl₃) δ: 7.78 (s, 1H), 7.28 (s, 2H), 6.94-7.04 (m, 4H), 6.43 (d, J=3.6 Hz, 1H), 6.30 (d, J=3.2 Hz, 1H), 1.90-1.96 (m, 1H), 0.90-0.93 (m, 2H), 0.61-0.65 (m, 2H).

### Step 3

A suspension of 6c (21.3 g, 41.0 mmol), bis(pinacolato)diboron (12.5 g, 49.3 mmol), KOAc (10.0 g, 102.6 mmol) and Pd(dppf)Cl₂ (3 g, 4.1 mmol) in dioxane (250 mL) was allowed to stir at 100° C. for 2 hours under N₂ atmosphere. The reaction mixture was cooled and concentrated in vacuo, the residue obtained was purified using Flash column chromatography on silica gel, eluting with petroleum ether:ethyl acetate (30:1) to provide 6d (20 g, 79% yield). LC/MS: Anal. Calcd. For [M+H]⁺ C34H38B2FNO5S; 614.26. found 614.4.

### Step 4

A suspension of 6d (8.3 g, 13.5 mmol), cap 31 (11.1 g, 29.8 mmol), Na₂CO₃ (7.2 g, 67.7 mmol) and Pd(dppf)Cl₂ (0.99 g, 1.35 mmol) in THF/H₂O (v/v=5/1, 180 mL) was allowed to stir at 80° C. for about 15 hours under N₂ atmosphere. After that, the mixture was washed with water and extracted with ethyl acetate, washed with brine and dried over anhydrous sodium sulfate. After being concentrated in vacuo, the residue obtained was purified using Flash column chromatography on silica gel, eluting with petroleum ether:ethyl acetate (3:1 to 1:2) to provide 6e (5.6 g, 45% yield). LC/MS: Anal. Calcd. For [M+H]⁺ C50H56FN9O7S: 946.40. found 946.5.

### Step 5

Compound 6 was got from compound 6e (16.7 g) by SFC by using the following conditions:

Instrument: Thar SFC

Column: AS-H, 150×4.6 mm, 5 um

Mobile phase: A for CO₂ and B for IPA (0.05% DEA)

Gradient: B 5% to 40 for A

Flow rate: 2.5 mL/min

Back pressure: 100 bar

Column temperature: 35° C.

Wavelength: 230 nm

Compound 6 (4.8 g, 29% yield). ¹H NMR (MeOD) δ: 8.02 (s, 1H), 7.95 (s, 1H), 7.78 (d, J=19.6 Hz, 2H), 7.46-7.57 (m, 2H), 7.39 (d, J=10.8 Hz, 1H), 7.31 (s, 1H), 7.19 (d, J=2.4 Hz, 1H), 6.42-6.53 (m, 2H), 5.21 (m, 2H), 4.20 (t, J=7.2 Hz, 2H), 4.08 (m, 2H), 3.79-3.92 (m, 2H), 3.64 (s, 6H), 2.55 (m, 2H), 1.97-2.31 (m, 8H), 1.89-1.96 (m, 1H), 0.81-1.01 (m, 14H), 0.53 (d, J=3.52 Hz, 2H). LC/MS: Anal. Calcd. For [M+H]⁺ C50H56FN9O7S; 946.40. found 946.3.

### Example 36

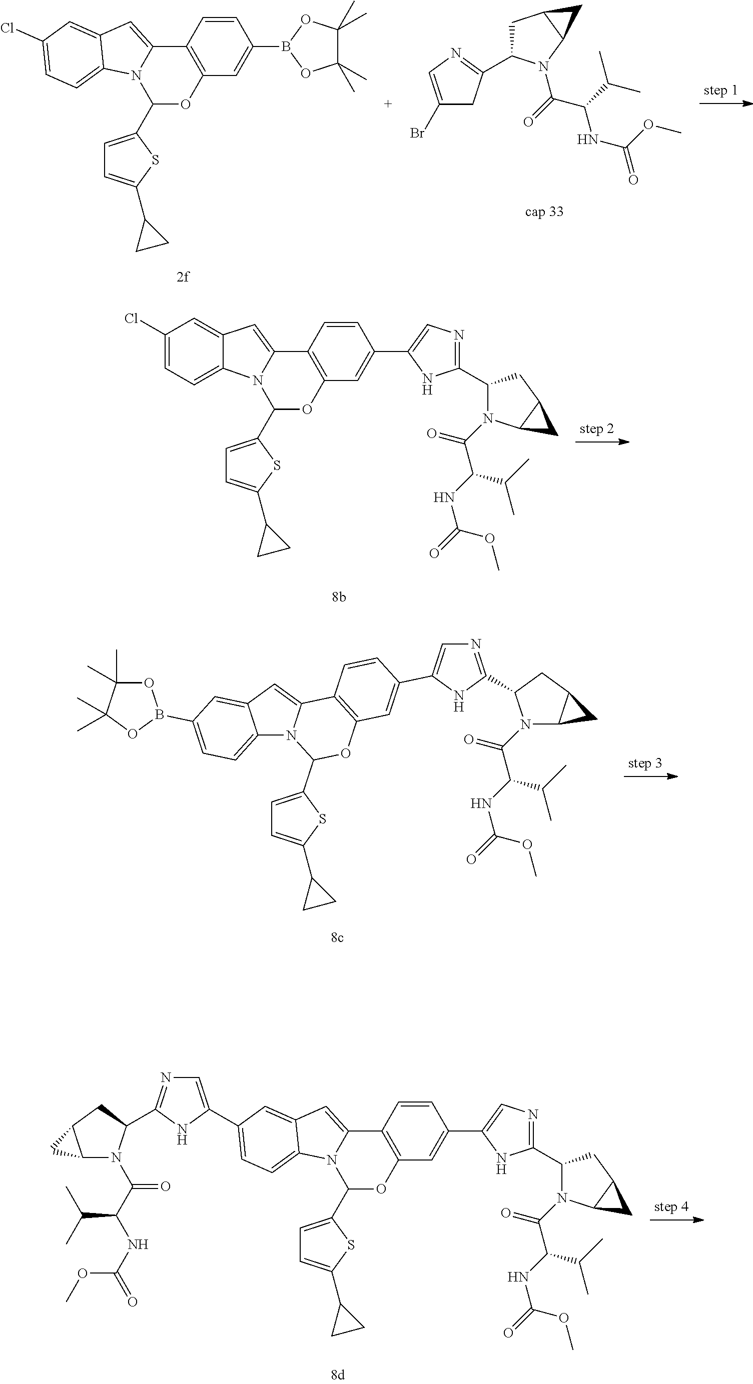

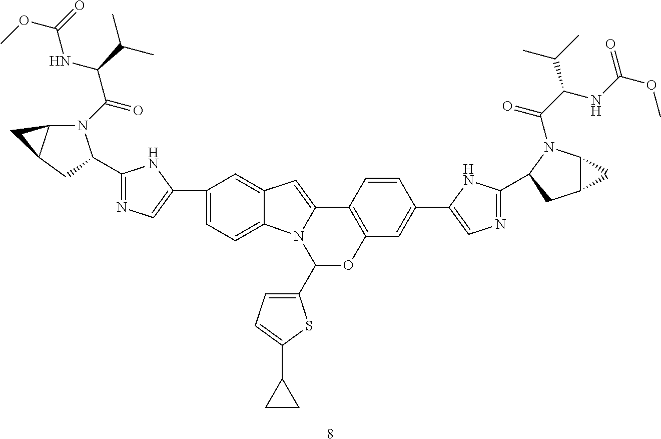

### Step 1

Compound 2f was made from Example 33. A suspension of 2f (5 g, 10 mmol), cap 33 (3.8 g, 10 mmol), Na₂CO₃ (2.7 g, 25 mmol) and Pd(dppf)Cl₂ (366 mg, 0.5 mmol) in THF/H₂O (v/v=5/1, 100 mL) was allowed to stir at 80° C. for about 15 hours under N₂ atmosphere. After that, the mixture was washed with water and extracted with ethyl acetate, washed with brine and dried over anhydrous sodium sulfate. After being concentrated in vacuo, the residue obtained was purified using Flash column chromatography on silica gel, eluting with petroleum ether:ethyl acetate (3:1 to 1:1) to provide 8b (5.6 g, 81% yield). LC/MS Anal. Calcd. For [M+H]⁺ C37H36ClN5O4S; 682.22. found 682.2.

### Step 2

To a mixture of 8b (3 g, 4.40 mmol), bis(pinacolato)diboron (1.34 g, 5.28 mmol), KOAc (1.1 g, 11 mmol), Pd₂(dba)₃ (364 mg, 0.352 mmol), X-Phos (336 mg 0.704 mmol) degassed and sealed under N₂ was added dry dioxane. Following further N₂ purging. The mixture was allowed to stir at 100° C. for about 15 hours. After that, cooling to 25° C., concentrated in vacuo and the residue obtained was purified using column chromatography on silica gel, eluting with petroleum ether: ethyl acetate (3:1 to 1:1) to provide 8c (2.8 g, 82% yield). LC/MS Anal. Calcd. For [M+H]⁺ C43H48BN5O6S; 774.35. found 774.4.

### Step 3

A mixture of 8c (2.8 g, 3.62 mol), cap 33 (1.7 g, 4.42 mmol), Na₂CO₃ (0.96 g, 9.05 mol) and Pd(dppf)Cl₂ (211 mg, 0.29 mmol) in THF/H₂O (v/v=5/1, 50 mL) was allowed to stir at 80° C. under N₂ atmosphere for about 15 hours. After that, the mixture was washed with water and extracted with ethyl acetate, washed with brine and dried over anhydrous sodium sulfate. After being concentrated in vacuo, the residue obtained was purified using Flash column chromatography on silica gel, eluting with petroleum ether:ethyl acetate (3:1 to 1:2) to provide 8d (1.7 g, 50% yield). LC/MS Anal. Calcd. For [M+H]⁺ C52H57N9O7S; 952.41; 952.9.

### Step 4

Compound 8 was got from compound 8d (1.7 g) by SFC by using the following conditions:

Instrument: Thar SFC

Column: AS-H, 150×4.6 mm, 5 um

Mobile phase: A for CO₂ and B for MeOH (0.05% DEA)

Gradient: B 5% to 40 for A

Flow rate: 2.5 mL/min

Back pressure: 100 bar

Column temperature: 35° C.

Wavelength: 230 nm

Compound 8 (460 mg, 27% yield). ¹H NMR (MeOD) δ: 7.95 (s, 1H), 7.83-7.90 (m, 2H), 7.67-7.70 (m, 2H), 7.37-7.47 (m, 4H), 7.10 (s, 1H), 6.46 (s, 2H), 5.10-5.11 (m, 2H), 4.50-4.54 (m, 2H), 3.79-3.80 (m, 2H), 3.64 (s, 6H), 2.61-2.69 (m, 2H), 2.42-2.46 (m, 2H), 1.89-2.18 (m, 6H), 0.89-1.07 (m, 18H), 0.49-0.51 (m, 2H). Anal. Calcd. For [M+H]⁺ C52H57N9O7S; 952.41; 953.4.

The following compounds of the present invention were made using the methods described in the Example above and substituting the appropriate reactants or reagents.

| | | | Observed |
|---|---|---|---|
| ID | Structure | Isomer | [M + H]+ |
| 7 8 | 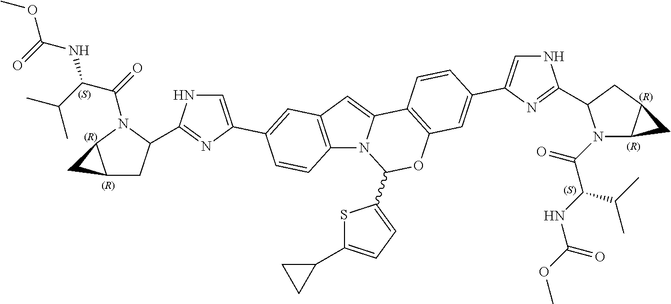 | Isomer 1 Isomer 2 | 952.6 952.8 |

### Example 37

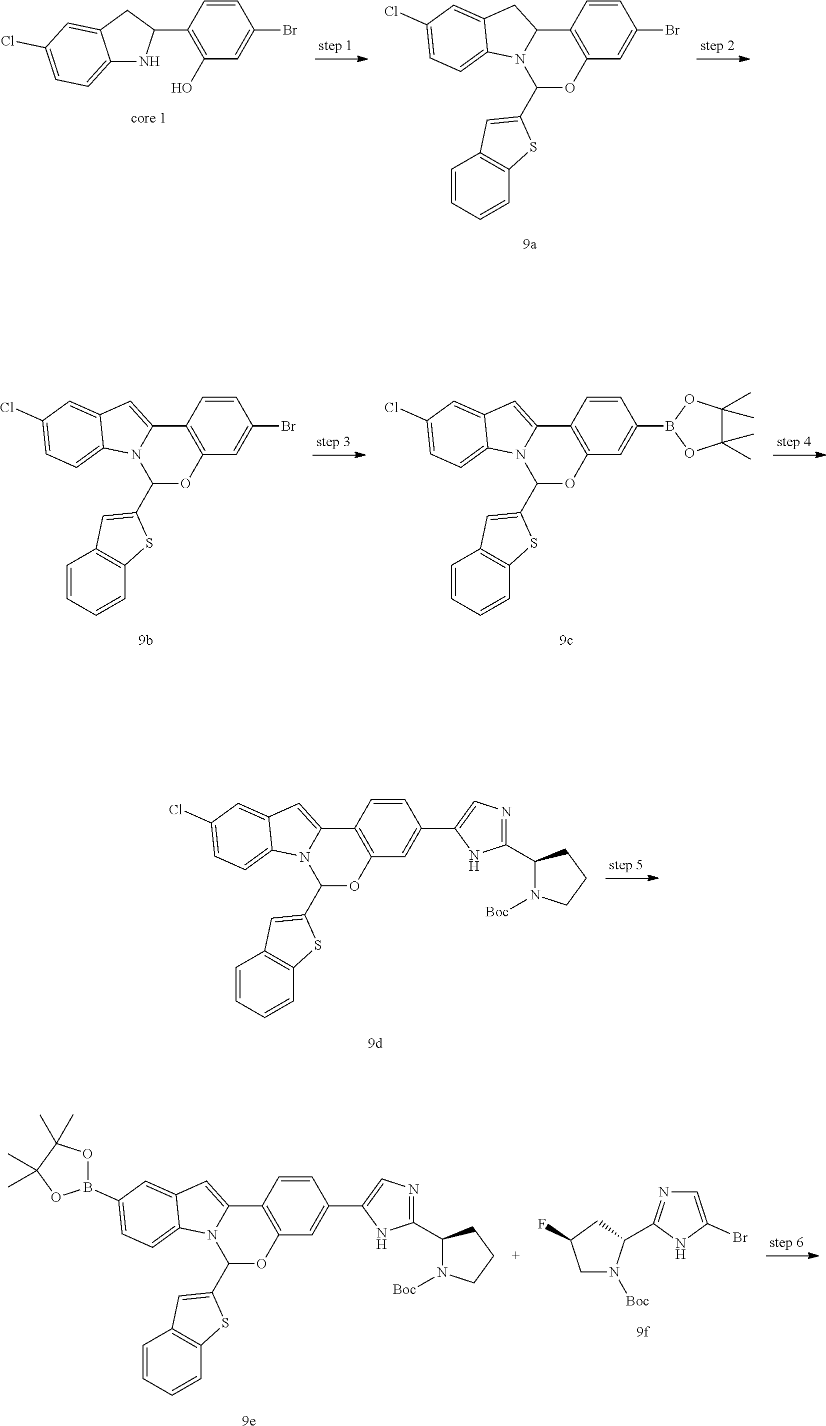

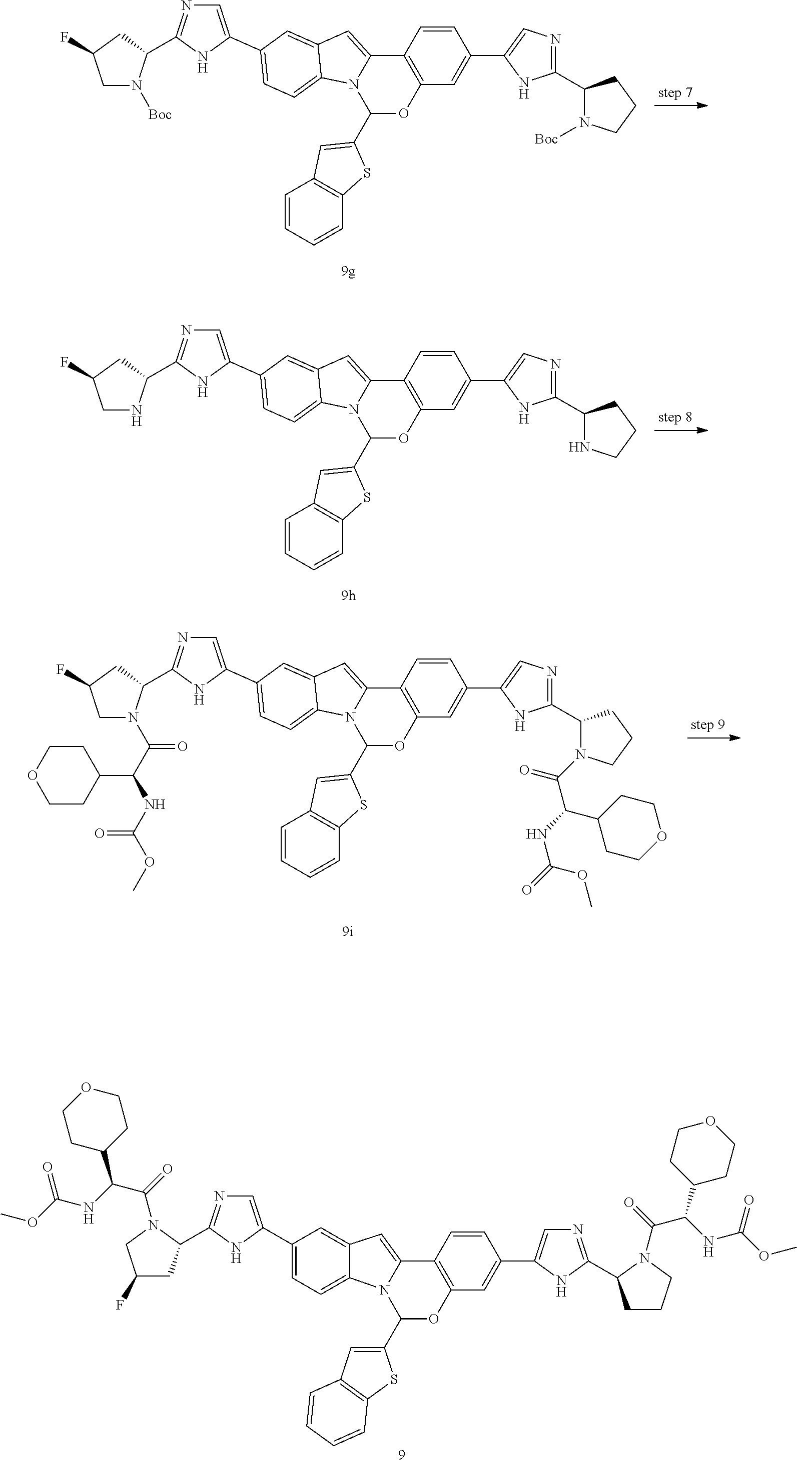

### Step 1

To a mixture of benzo[b]thiophene-2-carbaldehyde (3 g, 18.6 mmol) and core 1 (4 g, 12.4 mmol) in anhydrous CH₃CN (100 mL) was added TFA (424 mg, 3.7 mmol) at 25° C. The mixture was agitated for 6 hours. The reaction mixture became a clear solution and then a solid appeared. The solid was collected by filtration and washed with CH₃CN to provide 9a (5.4 g, 69% yield).

### Step 2

The solution of 9a (5.4 g, 11.7 mmol) in dry toluene (100 mL) was added DDQ (3.9 g, 17.2 mmol). After refluxing for 2 hours, the solvent was removed in vacuo and diluted with EtOAc. The organic layer was washed with saturated Na₂S₂O₃ aqueous and brine, dried over Na₂SO₄, filtered and concentrated in vacuo. The residue obtained was washed with MeOH (20 mL), filtered and the solid was 9b (4.8 g, 88% yield). ¹H NMR (CDCl₃): δ: 7.64-7.71 (m, 2H), 7.48-7.55 (m, 3H), 6.61-7.29 (m, 6H), 6.85 (s, 1H), 6.61 (s, 1H).

### Step 3

To a solution of 9b (4.8 g, 10.2 mmol) in dioxane (100 mL) was added bis pinacol borate (3.1 g, 12.3 mmol) and Pd(dppf)Cl₂ (372 mg, 0.51 mmol) and KOAc (2.9 g, 30.6 mmol). The reaction mixture was allowed to stir under N₂ and heated to 100° C. for about 15 hours. After that, the solvent was removed in vacuo, and the residue obtained was purified using Flash column chromatography on silica gel to provide 9c (4 g, 76% yield).

### Step 4

A suspension of 9c (3.4 g, 6.6 mmol), cap 32a (2.5 g, 7.9 mmol), Pd(dppf)Cl₂ (241 mg, 0.33 mmol) and Na₂CO₃ (2 g, 19.8 mmol) in THF/H₂O (5:1, 36 mL) was refluxed at 75° C. for about 15 hours under N₂ atmosphere. After that, the mixture was filtered, the filtrate was washed with water (50 mL) and extracted with EtOAc (100 mL), washed with brine and dried over anhydrous sodium sulfate. After being concentrated in vacuo, the residue obtained was purified using Flash column chromatography on silica gel (petroleum ether/ethyl acetate=8:1 to 3:1) to provide 9d (3.4 g, 82% yield).

### Step 5

To a mixture of 9d (3.1 g, 4.9 mmol), bis(pinacolato)diboron (1.5 g, 6.0 mmol), KOAc (1.4 g, 14.7 mmol), Pd₂(dba)₃ (228 mg, 0.25 mmol), X-Phos (238 g, 0.5 mmol) degassed and sealed under N₂ was added dry dioxane. Following further N₂ purging. The mixture was allowed to stir at 120° C. for about 15 hours. Under standard work-up to provide the residue which was purified using Flash column chromatography on silica gel to provide 9e (3 g, 85% yield).

### Step 6

Compound 9f was from Example 10 of International Publication No. 2012041014.

A suspension of 9e (3.5 g, 4.9 mmol), 9f (1.95 g, 5.9 mmol), Pd(dppf)Cl₂ (179 mg, 0.24 mmol) and Na₂CO₃ (1.56 g, 14.7 mmol) in THF/H₂O (5:1, 36 mL) was refluxed at 75° C. for about 15 hours under N₂ atmosphere. After that, the mixture was filtered; the filtrate was washed with water (50 mL) and extracted with EtOAc (100 mL), washed with brine and dried over anhydrous sodium sulfate. After being concentrated in vacuo, the residue obtained was purified using Flash column chromatography on silica gel (petroleum ether/ethyl acetate=5:1 to 1:1) to provide 9g (2.5 g, 60% yield).

### Step 7

Compound 9g (2.3 g, 2.7 mmol) was added into HCl/CH₃OH (20 mL, 3 mol/L). Then the mixture was allowed to stir at 25° C. for 2-3 hours. When the reaction completed, the mixture was concentrated in vacuo to provide 9 h used in next step directly.

### Step 8

To a mixture of 9 h (2.1 g, 3.3 mmol), acid (1.42 g, 6.5 mmol) and DIPEA (2 mL) in DMF (30 mL) was added BOP reagent (2.89 g, 6.5 mmol). The resulting mixture was allowed to stir at 25° C. for 16 hours before the solution was subjected directly to Pre-HPLC to provide 9i (1.5 g, 44% yield).

### Step 9

Compound 9 was separated from compound 9i (1.5 g) by SFC by using the following conditions:

Column: AS-H

Solvent: MeOH (0.05% DEA)/CO₂

Total flow: 2

Wavelength: 220 nm

Compound 9 (600 mg, 40% yield). ¹H NMR (MeOD) δ: 8.05 (s, 1H), 7.97-7.99 (m, 2H), 7.86 (s, 1H), 7.79 (s, 1H), 7.62-7.70 (m, 1H), 7.57-7.59 (m, 2H), 7.46-7.53 (m, 3H), 7.23-7.27 (m, 3H), 6.83 (m, 1H), 5.36-5.56 (m, 1H), 5.32-5.34 (m, 1H), 5.15-5.19 (m, 1H), 4.45-4.48 (m, 1H), 4.16-4.23 (m, 2H), 4.06-4.09 (m, 2H), 3.84-3.91 (m, 5H), 3.64 (m, 6H), 3.32 (m, 3H), 3.24 (m, 1H), 2.86-2.88 (m, 1H), 2.51 (m, 2H), 2.13-2.23 (m, 3H), 1.90-1.93 (m, 2H), 1.56-1.59 (m, 2H), 1.23-1.45 (m, 6H). LC/MS: Anal. Calcd. For [M+H]⁺ C55H58FN9O9S; 1040.17. found: 1040.1.

The following compounds of the present invention were made using the methods described in the Example above and substituting the appropriate reactants or reagents.

| | | | Observed |
|---|---|---|---|
| ID | Structure | Isomer | [M + H]+ |
| 757 | 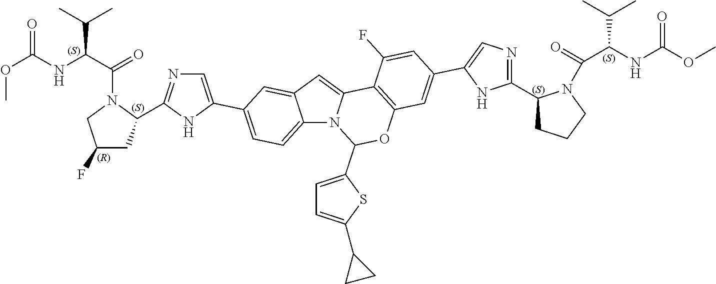 | racemic | 964.67 |

### Example 38

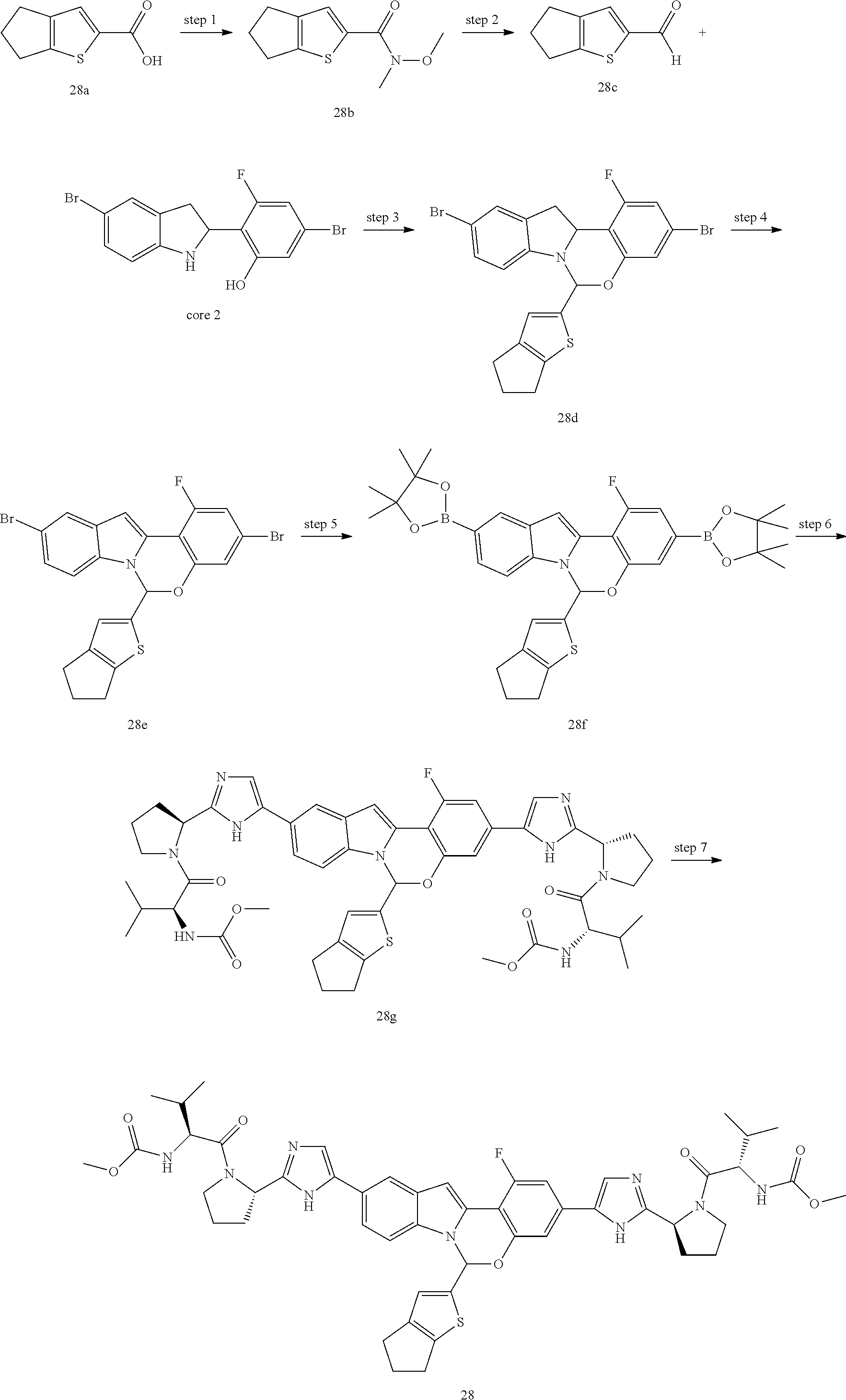

### Step 1

To a mixture of 28a (3.0 g, 20.0 mmol), compound N,O-dimethyldroxylamine hydrochloride (2.0 g, 20.6 mmol) and DIPEA (20.6 mmoL) in DMF (15 mL) was added HATU (7.6 g, 20.0 mmol). The resulting mixture was allowed to stir at 25° C. LCMS judged the starting material was consumed up. The crude product was purified using Flash column chromatography on silica gel (petroleum ether:ethyl acetate=8:1 to 5:1) to provide compound 28b (3.0 g, 80.2% yield).

### Step 2

A suspension of the compound 28b (3.0 g, 14.2 mmol) in THF (150 mL) was added LiAlH₄ (0.89 g, 22 mmol) under −75° C. After then, the temperature was rebound to 25° C. for 1 hour. The mixture was pouring to water, extracted with ethyl acetate. The organic layer was washed with water, brine and dried over Na₂SO₄, concentrated in vacuo to provide compound 28c (2.0 g, 92.1% yield).

### Step 3

A suspension of the 28c (2.0 g, 13.1 mmol) and core 2 (2.32 g, 6.0 mmol) in anhydrous CH₃CN (20 mL) was added TFA (0.3 mmol) at 25° C. The mixture was agitated for 6 hours at 25° C. The reaction mixture became a clear solution and then a solid appeared. The desired compound 28d was collected as a solid by filtration and washed with CH₃CN (2.0 g, 63.9% yield).

### Step 4

A suspension of the 28d (1.036 g, 2.0 mmol) in dry toluene (20 mL) was added DDQ (681 mg, 3.0 mmol). After refluxing for 2 hours, the solvent was removed in vacuo and diluted with EtOAc. The organic layer was washed with saturated Na₂S₂O₃ aqueous and brine, dried over Na₂SO₄, filtered and concentrated in vacuo. The residue obtained was washed with MeOH (10 mL), filtered and the solid just was the desired compound 28e (700 mg, 69.1% yield).

### Step 5

A suspension of the 28e (700 mg, 1.35 mmol) in dioxane was added bis pinacol borate (374 mg, 1.48 mmol) and Pd(dppf)Cl₂ (0.02 mmol), KOAc (2.7 mmol). The reaction mixture was allowed to stir under N₂ and heated to 110° C. for 2 hours. After that, the solvent was removed in vacuo, and the residue obtained was purified using Flash column chromatography on silica gel (petroleum ether:ethyl acetate=0 to 5:1) to provide the compound 28f (712 mg, 86% yield).

### Step 6

A suspension of the 28f (712 mg, 1.1 mmol), cap 31 (855 mg, 2.3 mmol), Na₂CO₃ (699 mg, 6.6 mmol) and Pd(dppf)Cl₂ (110 mg, 0.15 mmol) in THF/H₂O (5:1, 36 mL) was allowed to stir at 100° C. under N₂ atmosphere for about 15 hours. The reaction mixture was concentrated in vacuo and purified using Flash column chromatography on silica gel (petroleum ether:ethyl acetate=1:1 to 1:10) to provide compound 28g (220 mg, 20% yield).

### Step 7

Compound 28 was got from compound 28g (220 mg) by SFC separation using the following conditions:

Instrument: Thar SFC

Column: Chiralcel OD-3 150×4.6 mm I.D

Solvent: 40% of methanol (0.05% DEA) in CO₂

Flow rate: 2.5 mL/min

Wavelength: 340 nm

Compound 28 (91 mg, 41.3% yield). ¹H NMR (MeOD) δ: 7.78-7.91 (m, 1H), 7.17-7.46 (m, 7H), 6.84-6.99 (m, 1H), 6.24 (s, 1H), 5.12-5.19 (m, 2H), 4.19-4.31 (m, 2H), 3.87-4.05 (m, 4H), 3.59-3.64 (m, 6H), 2.03-2.65 (m, 17H), 0.90-0.95 (m, 12H). LC/MS: Anal. Calcd. For [M+H]⁺ C50H56FN9O7S; 946.40. found 946.5.

### Example 39

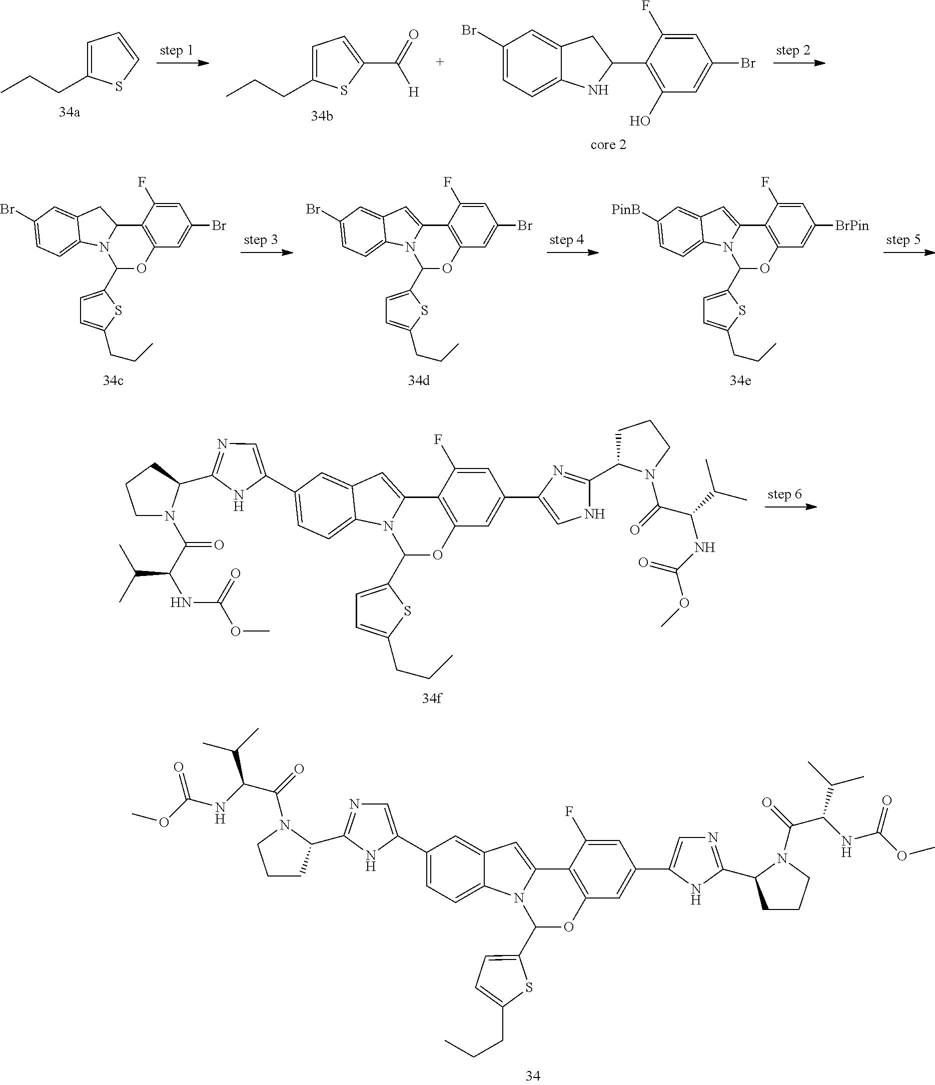

### Step 1

To a mixture of 34a (2.5 g, 20 mmol) in THF (30 mL) was added a 2.5 M solution of n-BuLi (8.8 mL, 22 mmol) at −78° C. under N₂. The mixture was agitated for 1 hour at this temperature then DMF (2.2 g, 30 mmol) was added. The mixture was allowed to stir at 25° C. for 3 hours. Quenched with NH₄Cl saturate solution and extracted with EtOAc. The organic layer was dried over sodium sulfate, and concentrated in vacuo and purified using Flash column chromatography on silica gel to provide the desired compound 34b (3 g, 82% yield). LC/MS: Anal. Calcd. For [M+H]⁺ C8H100S; 155.05. found 155.5.

### Step 2

To a mixture of 34b (0.516 g, 3.4 mmol) and core 2 (1 g, 2.6 mmol) in anhydrous CH₃CN (20 mL) was added TFA (0.089 g, 0.78 mmol) at 25° C. The mixture was agitated for 6 hours at 25° C. The reaction mixture became a clear solution and then a solid appeared. The solid was collected by filtration and washed with CH₃CN to provide compound 34c (1 g, 61% yield). LC/MS: Anal. Calcd. For [M+H]⁺ C22H18Br2FNOS; 523.94. found 524.01.

### Step 3

The solution of 34c (0.8 g, 1.5 mmol) in dry toluene (20 mL) was added DDQ (0.521 g, 2.3 mmol). After refluxing for 2 hours, the solvent was removed in vacuo and diluted with EtOAc. The organic layer was washed with saturated Na₂SO₃ aqueous and brine, dried over Na₂SO₄, filtered and concentrated in vacuo. The residue obtained was washed with MeOH (10 mL), filtered and the solid just was the product 34d (0.9 g, 89% yield). LC/MS: Anal. Calcd. For [M+H]⁺ C22H16Br2FNOS; 521.93. found 522.04.

### Step 4

To a solution of 34d (0.7 g, 1.3 mmol) in dioxane was added bis pinacol borate (0.69 g, 2.7 mmol) and Pd(dppf)Cl₂ (0.095 g, 0.13 mmol) and KOAc (0.510 g, 5.2 mmol). The reaction mixture was allowed to stir under N₂ and heated to 110° C. for about 15 hours. After that, the solvent was removed in vacuo, and the residue obtained was purified using Flash column chromatography on silica gel to provide the product 34e (0.7 g, 91% yield). LC/MS: Anal. Calcd. For [M+H]⁺ C34H40B2FNO5S; 616.28. found 616.51.

### Step 5

A suspension of 34e (800 mg, 1.3 mmol), cap 31 (0.970 g, 2.6 mmol), Pd(dppf)Cl₂ (95 mg, 0.13 mmol), Na₂CO₃ (0.551 g, 5.2 mmol) and in THF/H₂O (10:1, 34 mL) was refluxed at 95° C. for about 15 hours under N₂ atmosphere. After that, the mixture was filtered; the filtrate was washed with water (50 mL) and extracted with EtOAc (100 mL), washed with brine and dried over anhydrous sodium sulfate. After being concentrated in vacuo, the residue obtained was transferred to purification team to do HPLC purification to provide the desired compound 34f (500 mg, 40.5% yield).

### Step 6

Compound 34 was got from compound 34f (500 mg) by SFC by using the following conditions.

Column: Chiralpak AS-H 250×4.6 mm I.D.

Mobile phase: 40% of ethanol (0.05% DEA) in CO₂

Flow rate: 2.4 mL/min

Wavelength: 220 nm

Compound 34 (90 mg, 45%). ¹H NMR (MeOD) δ: 7.90 (s, 1H), 7.88 (s, 1H), 7.80 (s, 1H), 7.74 (s, 1H), 7.49-7.46 (m, 2H), 7.40-7.29 (m, 2H), 7.21 (s, 1H), 6.54-6.49 (m, 2H), 5.25-5.15 (m, 2H), 4.26-4.16 (m, 2H), 4.16-4.01 (m, 2H), 3.90-3.75 (m, 2H), 3.63 (m, 6H), 2.65-2.58 (m, 2H), 2.60-2.45 (m, 2H), 2.30-2.21 (m, 2H), 2.19-2.11 (m, 4H), 2.09-1.98 (m, 2H), 1.60-1.45 (q, 2H), 1.22 (s, 9H), 0.95-0.85 (m, 12H). LC/MS: Anal. Calcd. For [M+H]⁺ C50H58FN9O7S; 948.42. found 948.7.

### Example 40

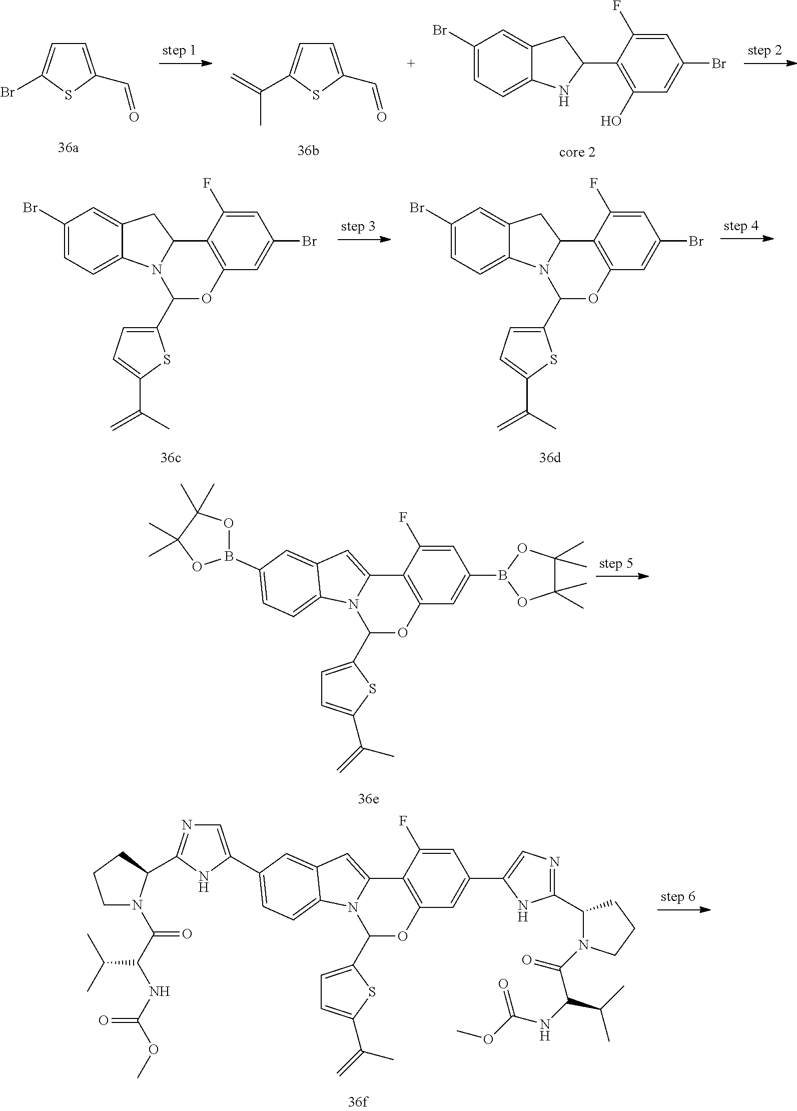

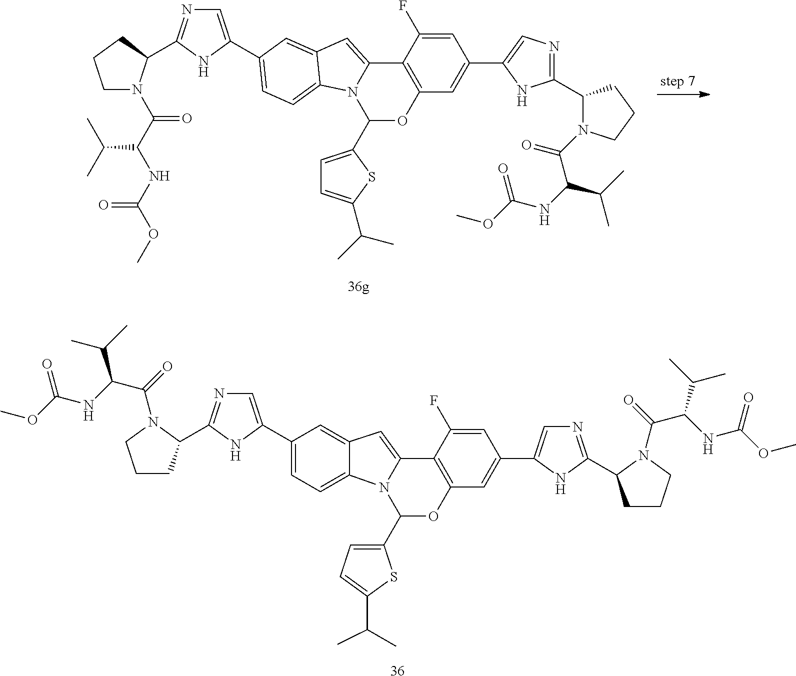

### Step 1

A mixture of 36a (20.4 g, 100 mmol), Isopropenylboronic acid pinacol ester (25 g, 149 mmol), Pd(PPh₃)₄ (0.51 g, 0.5 mmol), Na₂CO₃ (21 g, 198 mmol) in THF/H₂O (5:1, 480 mL) was refluxed at 75° C. for about 15 hours under N₂ atmosphere. After concentration to remove the solvent, the residue obtained was re-dissolved into water and ethyl acetate. The organic layer was dried over Na₂SO₄. After filtration and concentration, the residue obtained was purified using Flash column chromatography on silica gel (200 g, EtOAc/Hexane 0% to 5%) to provide 36b (10 g, 67% yield).

### Step 2

To the solution of core 2 (10.0 g, 26 mmol), 36b (7.9 g, 52 mmol) in anhydrous CH₃CN (55 mL) was added TFA (250 mg, 2.2 mmol) at 25° C. The mixture was allowed to stir for 20 hours at 25° C. The reaction mixture became a clear solution and then white solid appeared. The product 36c was collected by filtration and washed with CH₃OH (30 mL) (12.1 g, 89.3% yield). LC/MS: Anal. Calcd. For [M+H]⁺ C22H16Br2FNOS; 521.93. found 522.0.

### Step 3

A solution of 36c (12.4 g, 24 mmol) in dry toluene (100 mL) was added DDQ (6.78 g, 30 mmol). After refluxing for 2 hours, the solvent was removed in vacuo and diluted with EtOAc. The organic layer was washed with saturated Na₂SO₃ aqueous and brine, dried over Na₂SO₄, filtered and concentrated in vacuo. The residue obtained was washed with MeOH (50 mL), filtered and the solid just was the product 36d (6.74 g, 54.3% yield). LC/MS: Anal. Calcd. For [M+H]⁺ C22H14Br2FNOS; 519.91. found 520.0.

### Step 4

A suspension of 36d (4.0 g, 7.74 mmol), bis(pinacolato)diboron (5.85 g, 23.2 mmol), KOAc (4.6 g, 46.44 mmol) and Pd(dppf)Cl₂ (0.57 g, 0.774 mmol) in dioxane (150 mL) was allowed to stir at 115° C. under N₂ atmosphere 2 hours. The reaction mixture was cooled and concentrated in vacuo. The residue obtained was washed with water (70 mL) and extracted with EtOAc (100 mL), washed with brine and dried over anhydrous sodium sulfate. After being concentrated in vacuo, the crude product was SiO₂ chromatographied (petroleum ether/ethyl acetate=50/1 to 20/1) to provide compound 36e (3.5 g, 73.8% yield). LC/MS: Anal. Calcd. For [M+H]⁺ C34H38B2FNO5S; 614.26. found 614.30.

### Step 5

A suspension of 36e (5.0 g, 8.16 mmol), cap 31 (6.1 g, 16.32 mmol), Pd(dppf)Cl₂ (0.6 g, 0.816 mmol), Na₂CO₃ (5.2 g, 49 mmol) and in THF/H₂O (5:1, 240 mL) was refluxed at 75° C. for about 15 hours under N₂ atmosphere. After that, the mixture was concentrated in vacuo. The residue obtained was washed with water (80 mL) and extracted with EtOAc (200 mL), washed with brine and dried over anhydrous sodium sulfate. After being concentrated in vacuo, the residue obtained was purified using Flash column chromatography on silica gel (petroleum ether:ethyl acetate=5:1 to 1:1) to provide the product 36f (3.8 g, 49.3% yield). LC/MS: Anal. Calcd. For [M+H]⁺ C50H56FN9O7S; 946.40. found 946.70.

### Step 6

A suspension of 36f (1.7 g, 1.8 mmol) was dissolved in 200 mL of methanol. After addition of 10% Pd/C (170 mg), the mixture is hydrogenated by hydrogen balloon at 50° C. for 14 hours. The catalyst is filtered off through celite, the filtrate was concentrated in vacuo to provide the product 36g (1.69 g, 99% yield). LC/MS: Anal. Calcd. For [M+H]⁺ C50H58FN9O7S; 948.42. found 948.60.

### Step 6

Compound 36 was separated from compound 36g (2.2 g) by SFC by using the following conditions:

Column: Chiralpak AS-H 250×4.6 mm

Solvent: 40% of iso-propanol (0.05% DEA) in CO₂

Flow rate: 2.5 mL/min

Wavelength: 220 nm

Compound 36 (1.12 g, 50.9% yield). ¹H NMR (MeOD) δ: 8.0-7.97 (m, 1H), 7.83-7.74 (m, 3H), 7.51-7.26 (m, 4H), 7.16 (m, 1H), 6.58 (m, 1H), 6.52-6.50 (m, 1H), 5.24-5.19 (m, 2H), 4.24-4.22 (m, 2H), 4.09 (s, 2H), 3.87 (m, 2H), 3.65 (s, 6H), 3.01-2.99 (m, 1H), 2.56-2.50 (m, 2H), 2.56-2.50 (m, 8H), 1.18-1.17 (d, J=4 Hz, 6H), 0.94-0.89 (m, 12H). LC/MS: Anal. Calcd. For [M+H]⁺ C50H58FN9O7S; 948.42. found 948.60.

### Example 41

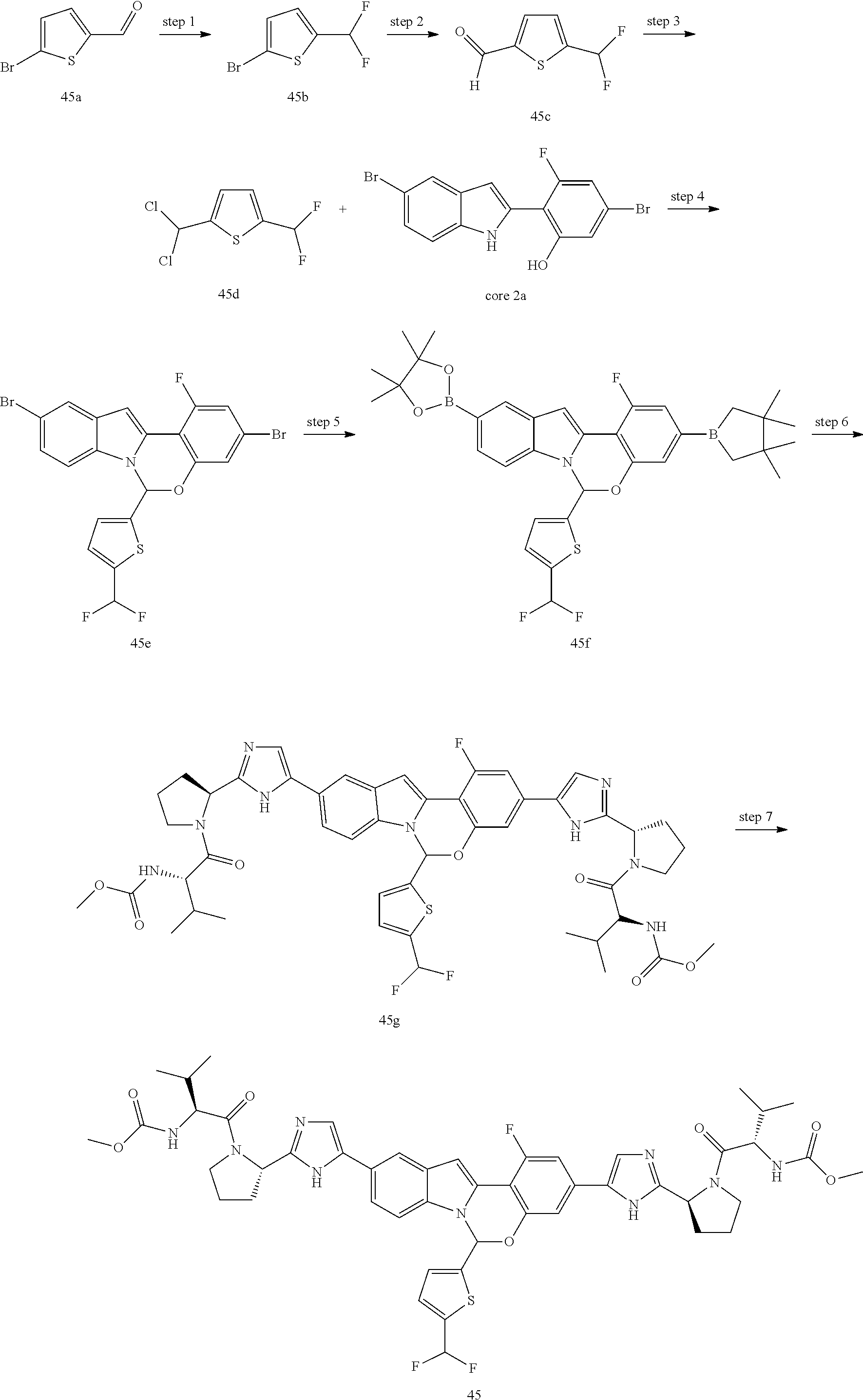

### Step 1

To a solution of 45a (4.9 g, 25 mmol) in CH₂Cl₂ (50 mL) at 0° C. DAST (8.84 g, 40 mmol) was added dropwise. Then the reaction mixture was allowed to stir at 25° C. for about 15 hours. After that, the mixture was quenched with saturated aqueous NaHCO₃. The organic layer was separated and dried over Na₂SO₄, the solvent was removed in vacuo and the residue obtained was purified using Flash column chromatography on silica gel elution with petroleum ether:ethyl acetate=20:1 to provide 45b as colorless oil (2.5 g, 50% yield). ¹H NMR (CDCl₃) δ: 7.03-6.99 (m, 2H), 6.86-6.58 (t, 1H).

### Step 2

To a solution of 45b (1.06 g, 5 mmol) in THF 25 mL at −78° C. under N₂ atmosphere, was added dropwise n-BuLi (2.4 mL, 6 mmol) at −78° C. within 5 minutes and the mixture was allowed to stir at the same temperature for half hour and DMF (730 mg) was added and the resulting mixture was allowed to stir at the same temperature for half hour. The reaction mixture was quenched with aqueous NH₄Cl and extracted with extracted with EtOAc (50 mL). The solvent was removed in vacuo and the residue obtained was purified using Flash column chromatography on silica gel elution with petroleum ether:ethyl acetate=30:1 to provide 45c as brown oil (760 mg, 80% yield).

### Step 3

Pyridine (158 mg, 2 mmol) was added to a solution of 45c (2.08 g, 10 mmol) in DCM (50 mL) at −10° C. followed by addition of PCl₅ (2.08 g, 10 mmol) all at once, stirred at −10° C. for another half hour. Checked by TLC, NaHCO₃ (2.52 g, 30 mmol) was added as solid to the reaction mixture. Stirred another 30 minutes; then filtered through celite, washed with more DCM. The filtrate was concentrated in vacuo and purified using a short SiO₂ column to provide 45d as clear oil.

### Step 4

Compound core 2a was made using the methods described in International Publication No. 2012041014.

A mixture of core 2a (3.85 g, 10 mmol), 45d (2.8 g, 10 mmol) and Cs₂CO₃ (9.75 g, 30 mmol) in 50 mL of DMSO was heated to 100° C. for 4 hours. The mixture was concentrated in vacuo and dissolved with DCM and water. The aqueous phase was extracted with DCM. The combined organic phase was washed with brine, dried over Na₂SO₄ and concentrated in vauco. The resulting residue was purified using Flash column chromatography on silica gel eluted with petroleum ether:ethyl acetate=20/1 to 10/1 to provide 45e (1 g, 20% yield).

### Step 5

To a solution of 45e (1.6 g, 3 mmol) in 1,4-dioxane was added bis pinacol borate (1.7 g, 6.6 mmol), Pd(dppf)Cl₂ (219 mg, 0.3 mmol) and KOAc (1.18 g, 12 mmol). The reaction mixture was allowed to stir under N₂ and heated to 110° C. for about 15 hours. The solvent was removed in vacuo, and the residue obtained was purified using Flash column chromatography on silica gel to provide 45f (0.7 g, 38.8% yield).

### Step 6

A suspension of 45f (626 mg, 1 mmol), methyl ((S)-1-((S)-2-(5-bromo-1H-imidazol-2-yl)pyrrolidin-1-yl)-3-methyl-1-oxobutan-2-yl) carbamate (746 mg, 2 mmol), Na₂CO₃ (424 mg, 4 mmol) and Pd(dppf)Cl₂ (73 mg, 0.1 mmol) in THF/H₂O (5/1, 12 mL) was allowed to stir at 100° C. under N₂ atmosphere for about 15 hours. The reaction mixture was concentrated in vacuo and purified using Flash column chromatography on silica gel to provide 45g (156 mg, 16% yield).

### Step 7

Compound 45 was separated by SFC from compound 45g (156 mg) by using the following conditions:

Column: Chiralpak AS-H 250×4.6 mm

Solvent: 40% of iso-propanol (0.05% DEA) in CO₂

Flow rate: 2.5 mL/min

Wavelength: 220 nm

Compound 45 (50 mg, 32% yield). ¹H NMR (MeOD): δ: 8.05 (s, 1H), 8.03 (s, 1H), 7.92 (s, 1H), 7.78 (s, 1H), 7.70 (d, J=8 Hz, 1H), 7.62-7.56 (m, 2H), 7.46-7.37 (m, 2H), 7.24 (s, 1H), 7.11 (s, 1H), 6.99-6.72 (t, 1H), 6.66 (s, 1H), 5.26-5.18 (m, 2H), 4.23-4.19 (m, 2H), 4.09 (m, 2H), 3.87-3.85 (m, 2H), 3.65 (s, 6H), 2.59-2.00 (m, 10H), 0.94-0.87 (m, 12H). LC/MS: Anal. Calcd. For [M+H]⁺ C48H52F3N9O7S: 956.04. found 956.

### Example 42

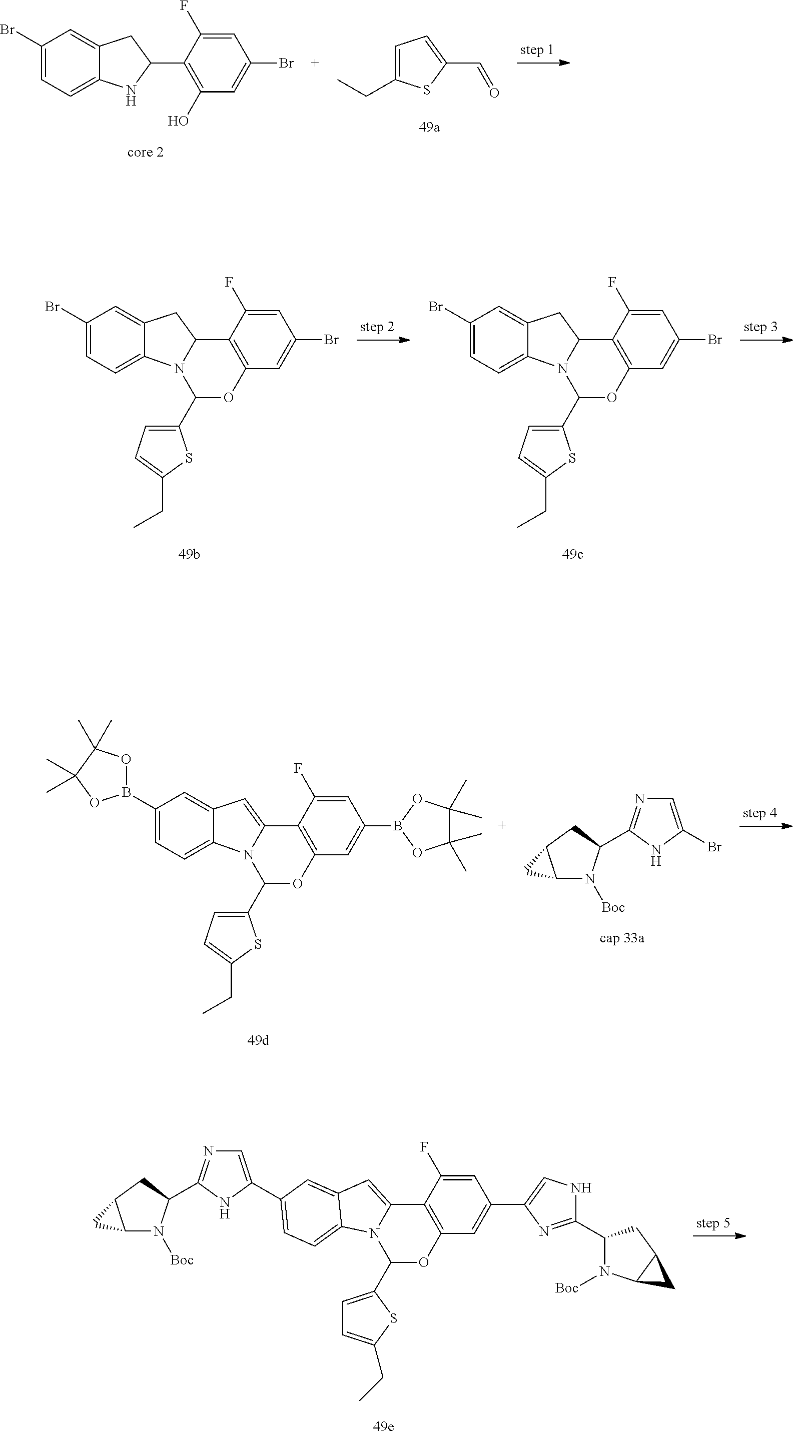

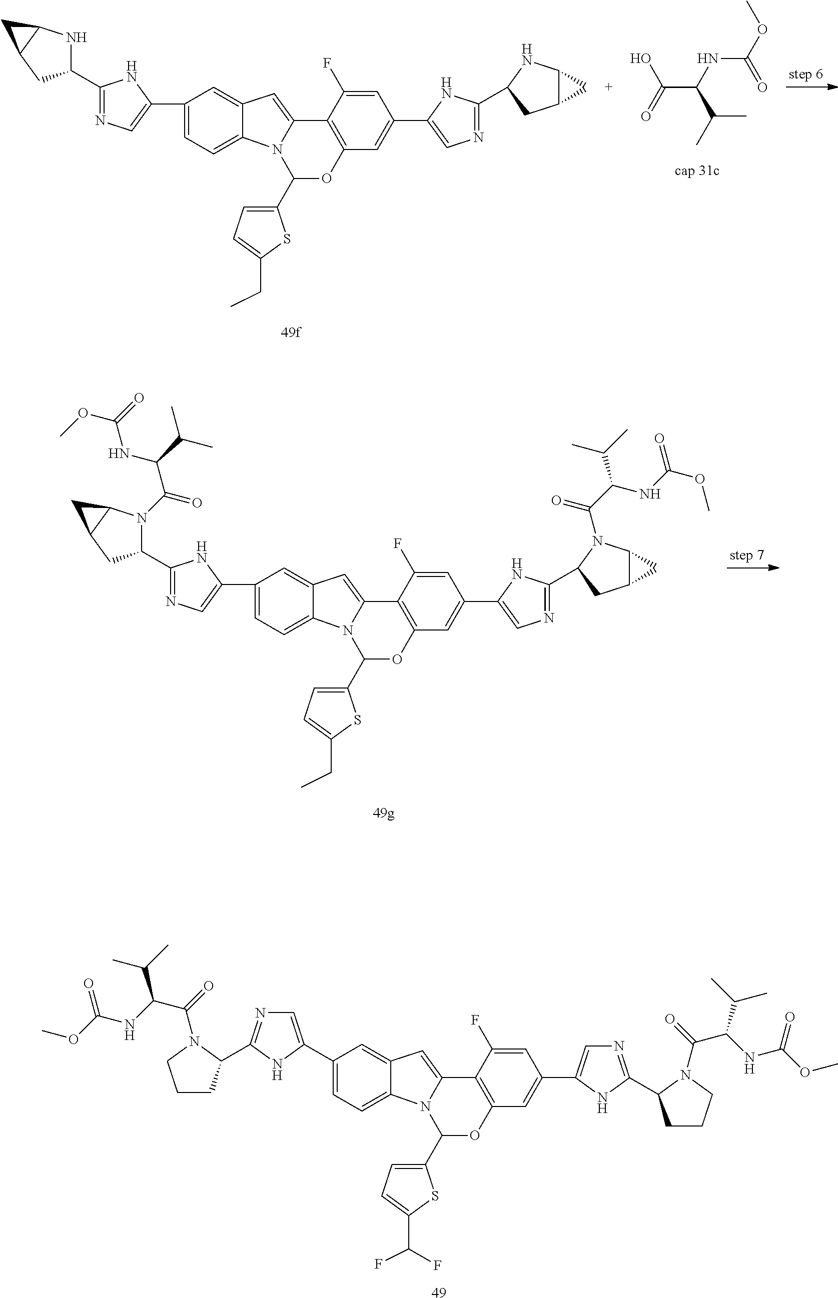

### Step 1

TFA (60 mg, 0.5 mmol) was added to a solution of core 2 (2.0 g, 5.1 mmol) and compound 49a (1.1 g, 8 mmol) in anhydrous CH₃CN (40 mL) at 25° C. The mixture was allowed to stir 2 hours at 25° C., during which time, the reaction mixture became a clear solution and then a solid appeared. The solid was collected by filtration, washed with CH₃CN and dried in vacuo to provide compound 49b (2.1 g, 80% yield).

### Step 2

A solution of compound 49b (2.0 g, 4 mmol) in dry toluene (30 mL) was added DDQ (1.35 g, 6 mmol). After refluxing for 2 hours, the solvent was removed in vacuo and diluted with EtOAc. The organic layer was washed with saturated Na₂SO₃ aqueous and brine, dried over Na₂SO₄, filtered and concentrated in vacuo. The residue obtained was washed with MeOH (10 mL), filtered and the collected solid was dried to provide compound 49c (1.6 g, 74% yield).

### Step 3

A suspension of the compound 49c (1.6 g, 3.2 mmol), bis(pinacolato)diboron (2.3 g, 8 mmol), KOAc (1.6 g, 16 mmol) and Pd(dppf)Cl₂ (0.3 g, 0.4 mmol) in dioxane (25 mL) was allowed to stir at 100° C. under N₂ atmosphere for 2 hours. The reaction mixture was cooled and concentrated in vacuo, and the crude product was purified using Flash column chromatography on silica gel (petroleum ether:ethyl acetate=95:5 to 80:20) to provide the compound 49d (1.25 g, 66% yield).

### Step 4

Compound cap 33a was prepared in Example 12A of 2012041014. A suspension of the compound 49d (2 g, 3.32 mmol), cap 33a (2.4 g, 7.31 mmol), Na₂CO₃ (1.4 g, 13.28 mmol) and Pd(dppf)Cl₂ (243 mg, 0.33 mmol) in THF/H₂O (5:1, 48 mL) was allowed to stir at 100° C. under N₂ atmosphere for about 15 hours. LCMS and TLC were detected the reaction. Separated the water phase through the reparatory funnel, and the organic phase was concentrated in vacuo and purified using Flash column chromatography on silica gel (eluent:petroleum ether:ethyl acetate=2:1 to 1:1) to provide the desired compound 49e (1.23 g, 44%). LC/MS: Anal. Calcd. For [M+H]⁺ C47H50FN7O5S; 844.36. found 844.5.

### Step 5

To a solution of compound 49e (1.22 g, 1.44 mmol) in dry dioxane (3 mL) was added HCl-dioxane (6 mL) through syringe and stirred at 25° C. for 2 hours, then concentrated in vacuo and dried under high vacuum to provide HCl salt of the desired product compound 49f (0.93 g, 99%). LC/MS: Anal. Calcd. For [M+H]⁺ C37H34FN7OS; 643.25. found 644.4.

### Step 6

Compound cap 31c was prepared in Example 1 of International Publication No. 2012041014.

To a mixture of the compound 49f (930 mg, 1.44 mmol), cap 8c (505.4 mg, 2.88 mmol) and DIPEA (371.5 mg, 2.88 mmol) in DMF (6 mL) was added HATU (1.09 g, 2.88 mmol). The resulting mixture was allowed to stir at 25° C. for 2 hours before the solution was transferred to purification team to do HPLC purification to provide the desired compound 49g (750 mg, 54%). LC/MS: Anal. Calcd. For [M+H]⁺ C51H52FN9O7S; 958.37. found 958.5.

### Step 7

The compound 49 was got from compound 49g (750 mg) by SFC separation by using the following conditions:

Column: Chiralpak AS-H 250×4.6 mm I.D., 5 um

Mobile phase: 50% of iso-propanol (0.05% DEA) in CO₂

Flow rate: 2.0 mL/min

Wavelength: 340 nm

Compound 49 (200 mg, 26% yield). ¹H NMR (MeOD) δ: 8.02 (s, 1H), 7.95 (s, 1H), 7.84 (s, 1H), 7.75 (s, 1H), 7.45-7.57 (m, 2H), 7.38 (d, J=11.0 Hz, 1H), 7.31 (s, 1H), 7.20 (d, J=2.7 Hz, 1H), 6.52 (dd, J=18.8, 3.1 Hz, 2H), 5.01-5.17 (m, 2H), 4.51 (dd, J=12.1, 6.7 Hz, 2H), 3.64 (s, 6H), 2.61-2.76 (m, 4H), 2.44 (dt, J=14.2, 6.8 Hz, 2H), 1.98-2.23 (m, 4H), 0.86-1.16 (m, 21H). MS (ESI) m/e (M+H+): 954. LC/MS: Anal. Calcd. For [M+H]+ C51H52FN9O7S; 958.37. found 958.5.

### Example 43

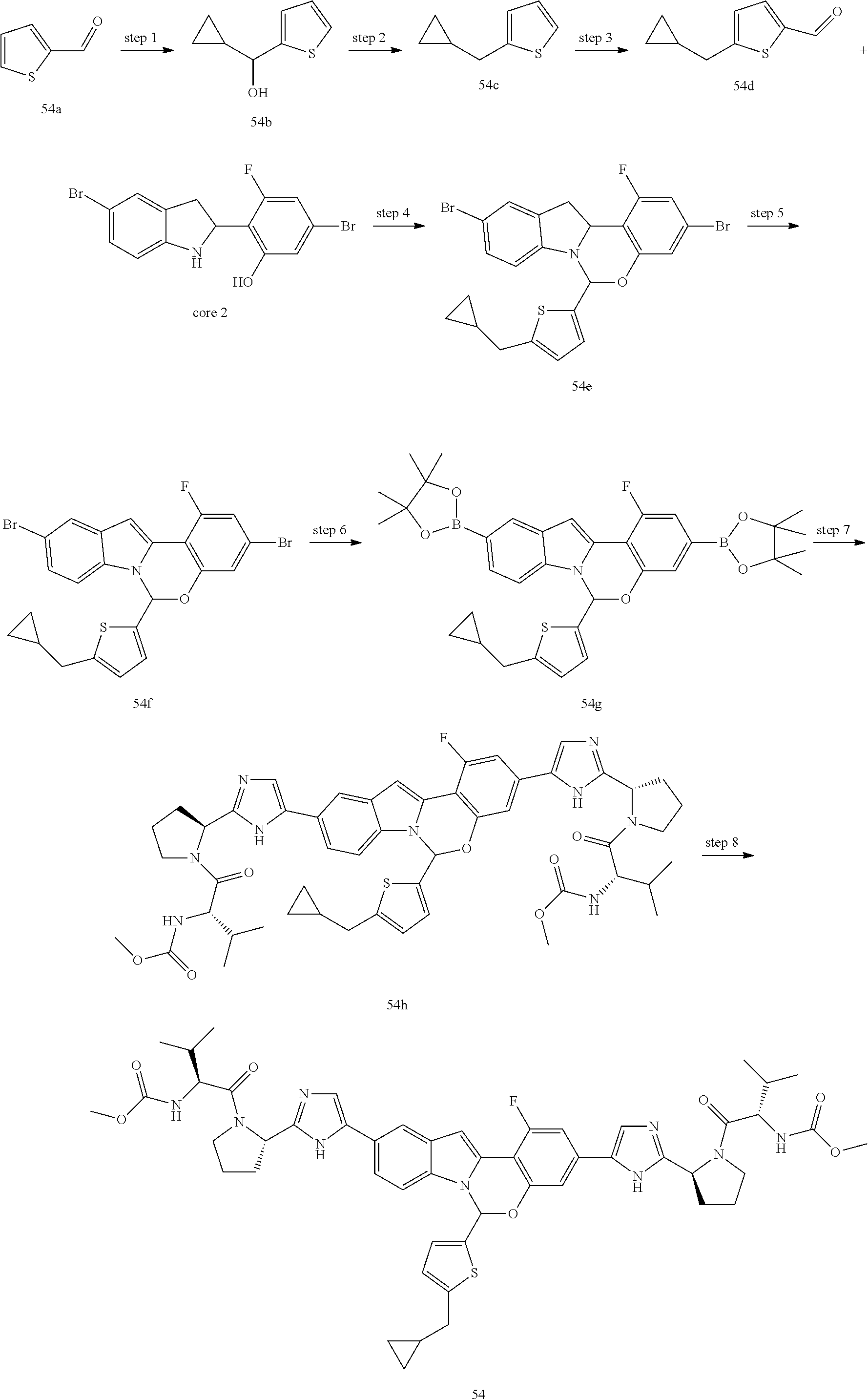

### Step 1

To a solution of compound 54a (2.8 g, 25 mmol) in anhydrous THF(100 mL) was added cyclopropylmagnesium bromide (60 mL, 0.5 mmol/mL) dropwise at −78° C. under nitrogen atmosphere. After addition, the reaction was allowed to stir at −78° C. for 3 hours. Detected by TLC, the starting material was consumed up. Quenched by saturated ammonium chloride solution, extracted with ethyl acetate, dried over anhydrous sodium sulfate, concentrated in vacuo and purified using Flash column chromatography on silica gel eluting with petroleum/ethyl acetate (100/1-10/1) to provide 54b (2.9 g, 75% yield). ¹H NMR (CDCl₃): 7.28 (m, 1H), 7.06, 7.05 (d, 1H, J=3.2 Hz), 6.99-6.97 (m, 1H), 4.27-4.25 (m, 1H), 2.03-2.02 (m, 1H), 1.36-1.25 (m, 1H), 0.68-0.63 (m, 2H), 0.55-0.45 (m, 2H).

### Step 2

To a solution of compound 54b (4.6 g, 30 mmol) in dichloromethane (90 mL) was added TFA (17.1 g, 150 mmol) and Et₃SiH (8.7 g, 75 mmol) at −20° C. Then the mixture was allowed to stir at 25° C. for 1 hour, the starting material was consumed up by checking TLC. Diluted with dichloromethane, washed with water and brine, dried over anhydrous sodium sulfate, concentrated in vacuo and purified using Pre-HPLC to provide compound 54c (0.8 g, 20% yield). ¹H NMR (CDCl₃): 7.14-7.12 (m, 1H), 6.94-6.92 (m, 1H), 6.86-6.85 (m, 1H), 4.27-4.25 (m, 1H), 2.75-2.73 (m, 2H), 1.08-1.04 (m, 1H), 0.59-0.55 (m, 2H), 0.27-0.23 (m, 2H).

### Step 3

To a solution of compound 54c (0.8 g, 5.8 mmol) in anhydrous THF (30 mL) was added n-BuLi (2.8 mL, 6.9 mmol) dropwise under nitrogen atmosphere at −78° C. When the addition was finished, the mixture was allowed to stir for an additional 1 hour at −78° C. DMF (2 mL, 23.2 mmol) was added below −70° C. The mixture was allowed to stir at −78° C. for 2 hours. Citric acid was added to the reaction mixture. The mixture was poured into water. The aqueous was extracted with ethyl acetate. The product was dried over anhydrous sodium sulfate and concentrated in vacuo. The reaction mixture was purified using Flash column chromatography on silica gel to 54d as oil (0.45 g, 50% yield). ¹H NMR (CDCl₃): 9.83 (s, 1H), 7.63, 7.62 (d, 1H, J=3.6 Hz), 6.98, 6.97 (d, 1H, J=4.0 Hz), 2.79, 2.77 (d, 1H, J=2.8 Hz), 1.09-1.05 (m, 1H), 0.63-0.61 (m, 2H), 0.29-0.27 (m, 2H).

### Step 4

To a mixture of 54d (350 mg, 2.1 mmol) and core 2 (625 mg, 1.6 mmol) in anhydrous CH₃CN (8 mL) was added TFA (55 mg, 0.48 mmol) at 25° C. The mixture was agitated for 6 hours at 25° C. The reaction mixture became a clear solution and then a solid appeared. The solid was collected by filtration and washed with CH₃CN to provide compound 54e (0.6 g, 70% yield). LC/MS: Anal. Calcd. For [M+H]⁺ C23H19Br2 FNOS; 535.95. found 536.2.

### Step 5

The solution of 54e (580 mg, 1.08 mmol) in dry toluene (20 mL) was added DDQ (368 mg, 1.62 mmol). After refluxing for 2 hours, the solvent was removed in vacuo and diluted with EtOAc. The organic layer was washed with saturated Na₂S₂O₃ aqueous and brine, dried over Na₂SO₄, filtered and concentrated in vacuo. The residue obtained was washed with MeOH (10 mL), filtered to provide compound 54f (0.5 g, 83%). LC/MS: Anal. Calcd. For [M+H]+ C23H17 Br2FNOS: 533.94. found 534.26.

### Step 6

To a solution of compound 54f (480 mg, 0.9 mmol) in dioxane was added bis pinacol borate (548 mg, 2.16 mmol) and Pd(dppf)Cl₂ (66 mg, 0.09 mmol) and KOAc (353 mg, 3.6 mmol). The reaction mixture was allowed to stir under N₂ and heated to 110° C. for about 15 hours. After that, the solvent was removed in vacuo, and the residue obtained was purified using Flash column chromatography on silica gel to provide compound 54g (0.5 g, 90%). LC/MS: Anal. Calcd. For [M+H]+ C35H41B2FNO5S; 628.29. found 628.4.

### Step 7

A suspension of 54g (500 mg, 0.8 mmol), cap 31a (597 mg, 1.6 mmol), Pd(dppf)Cl₂ (59 mg, 0.08 mmol), Na₂CO₃ (339 mg, 3.2 mmol) and in THF/H₂O (10:1, 33 mL) was refluxed at 75° C. for about 15 hours under N₂ atmosphere. After that, the mixture was filtered; the filtrate was washed with water (50 mL) and extracted with EtOAc (100 mL), washed with brine and dried over anhydrous sodium sulfate. After being concentrated in vacuo, the residue obtained was purified using Pre-HPLC to provide compound 54h (300 mg, 50% yield).

### Step 8

Compound 54 was got from compound 54h (220 mg) by SFC separation by using the following conditions:

Column: Chiralpak AS-H 250×4.6 mm I.D., 5 um

Solvent: 40% of ethanol (0.05% DEA) in CO₂

Flow rate: 2.4 mL/min

Wavelength: 220 nm

Compound 54 (100 mg, 45.4% yield). ¹H NMR (MeOD) δ:8.00 (s, 1H), 7.87 (s, 1H), 7.82 (s, 1H), 7.75 (s, 1H), 7.54-7.30 (m, 4H), 7.18 (s, 1H), 6.60, 6.59 (d, 1H, J=3.6 Hz), 6.50, 6.49 (d, 1H, J=3.6 Hz), 5.26-5.16 (m, 2H), 4.23-4.19 (m, 2H), 4.08-4.06 (m, 2H), 3.86-3.83 (m, 2H), 3.64 (s, 6H), 2.57-2.48 (m, 4H), 2.26-2.02 (m, 8H), 0.93-0.87 (m, 13H), 0.45-0.43 (m, 2H), 0.11-0.10 (m, 2H). LC/MS: Anal. Calcd. For [M+H]+ C₅₁H₅₈FN₉O₇S; 960.42. found 960.12.

### Example 44

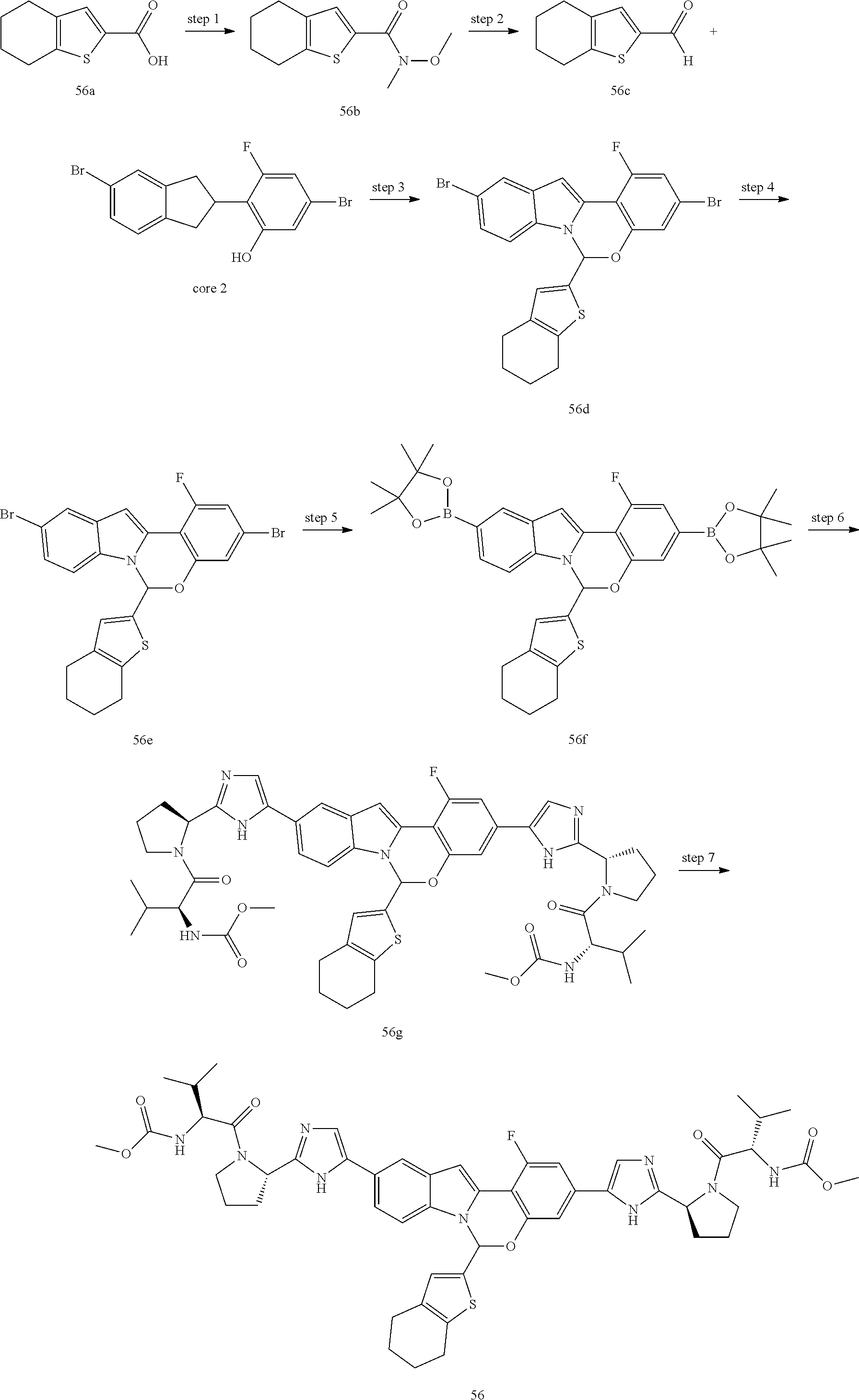

### Step 1

A solution of compound 56a (5 g, 27.5 mmol) and N,O-trimethylhydroxylamine (3.25 g, 33 mmol) in dichloromethane (100 mL) and triethylamine (6.6 g, 66 mmol) was added HATU(12.7 g, 33 mmol). The reaction was allowed to stir at 25° C. for about 15 hours. The solution was extracted with water and dichloromethane. The organic layer was washed with water, brine and dried over sodium sulfate, concentrated in vacuo to provide compound 56b (5.56 g, 90% yield). LC/MS: Anal. Calcd. For [M+H]⁺ C11H15NO2S; 226.08. found 226.0.

### Step 2

A suspension of compound 56b (5 g, 23.6 mmol) in THF (150 mL) was added LiAlH₄ (0.89 g, 22 mmol) under −55° C. After that the temperature was rebound to 25° C. for 3 hours. The mixture was pouring to water, extracted with ethyl acetate. The organic layer was washed with water, brine and dried over sodium sulfate, concentrated in vacuo to provide compound 56c (2 g, 54% yield). LC/MS: Anal. Calcd. For [M+H]⁺ C9H100S; 167.05. found 167.0.

### Step 3

To a mixture of the compound 56c (6 g, 36 mmol) and core 2 (6.8 g, 18 mmol) in anhydrous CH₃CN (100 mL) was added TFA (0.1 mmol) at 25° C. The mixture was agitated for 6 hours at 25° C. The reaction mixture became a clear solution and then a solid appeared. The compound 56d was collected by filtration as a solid and washed with CH₃CN (5 g, 54% yield). LC/MS: Anal. Calcd. For [M+H]⁺ C23H18Br2FNOS; 535.94. found 536.1.

### Step 4

The solution of compound 56d (5 g, 9.5 mmol) in dry toluene (100 mL) was added DDQ (4.3 g, 19 mmol). After refluxing for 2 hours, the solvent was removed in vacuo and diluted with EtOAc. The organic layer was washed with saturated Na₂S₂O₃ aqueous and brine, dried over Na₂SO₄, filtered and concentrated in vacuo. The residue obtained was washed with MeOH (10 mL), filtered and the solid just was the compound 56e (4.5 g, 90% yield). LC/MS: Anal. Calcd. For [M+H]⁺ C23H16Br2FNOS; 539.94. found 534.2.

### Step 5

A suspension of compound 56e (4.5 g, 8.4 mmol), bis(pinacolato)diboron (4.68 g, 18 mmol), KOAc (4.95 g, 0.54 mmol) and Pd(dppf)Cl₂ (0.612 g, 0.84 mmol) in dioxane (80 mL) was allowed to stir at 100° C. under N₂ atmosphere for about 15 hours. The reaction mixture was cooled, concentrated in vacuo and chromatographed on silica gel (petroleum ether/ethyl acetate=10/1) gave the compound 56f (4.49 g, 84.9% yield). LC/MS: Anal. Calcd. For [M+H]⁺ C35H40B2FNO5S; 628.28. found 628.1.

### Step 6

A suspension of compound 56f (1.5 g, 2.4 mmol), cap 31 (1.96 g, 5.3 mmol), Pd(dppf)Cl₂ (0.17 g, 0.24 mmol), Na₂CO₃ (1.53 g, 14.4 mmol) and in THF/H₂O (5:1, 36 mL) was refluxed at 80° C. for about 15 hours under N₂ atmosphere. After that, the mixture was filtered; the filtrate was washed with water (50 mL) and extracted with ethyl acetate (100 mL), washed with brine and dried over anhydrous sodium sulfate. After being concentrated in vacuo, the residue obtained was purified using Flash column chromatography on silica gel (petroleum ether:ethyl acetate=10:1) to provide compound 56g (1.5 g, 65.2% yield). LC/MS: Anal. Calcd. For [M+H]⁺ C51H58FN9O7S; 960.42. found 960.4.

### Step 7

Compound 56 was got from compound 56g (1.5 g) by SFC separation by using the following conditions:

Column: Chiralpak AS-H 250×4.6 mm I.D.,

Solvent: 50% of iso-propanol (0.05% DEA) in CO2

Flow rate: 2.0 mL/min

Wavelength: 220 nm

Compound 56 (0.5 g, 33% yield). ¹H NMR (MeOD) δ: 8.06 (s, 1H), 7.98-8.02 (m, 1H), 7.86 (s, 1H), 7.80 (s, 1H), 7.58 (s, 2H), 7.42 (d, J=11.0 Hz, 1H), 7.36 (s, 1H), 7.24 (br. s., 1H), 6.31 (s, 1H), 5.25 (dd, J=15.1, 7.0 Hz, 2H), 4.25 (t, J=7.5 Hz, 2H), 4.13 (br. s., 2H), 3.86-3.93 (m, 2H), 3.68 (s, 6H), 3.45 (d, J=12.0 Hz, 1H), 2.60 (br. s., 4H), 2.41 (br. s., 2H), 2.01-2.34 (m, 8H), 1.72 (d, J=17.1 Hz, 4H), 0.88-1.04 ppm (m, 12H).

### Example 45

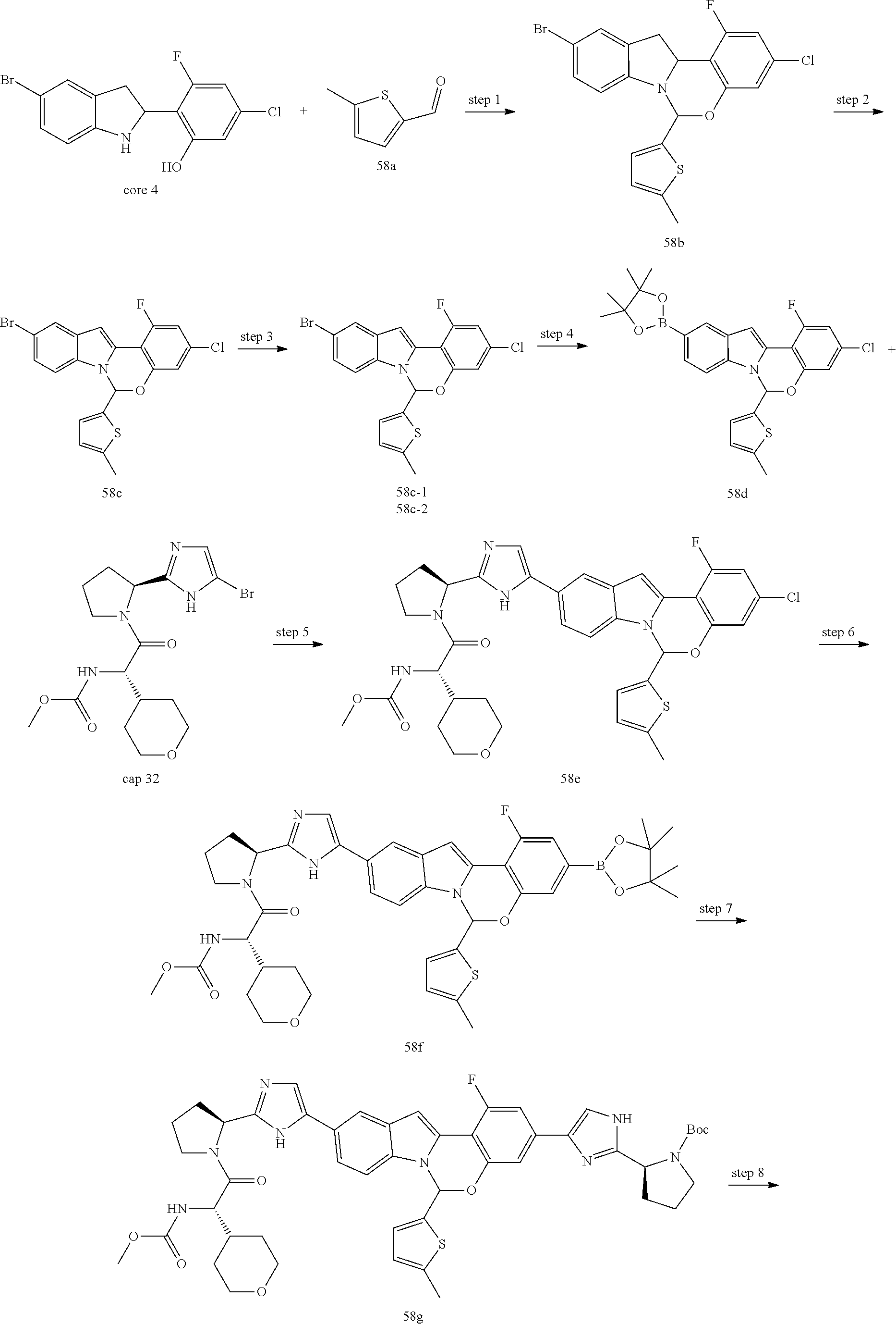

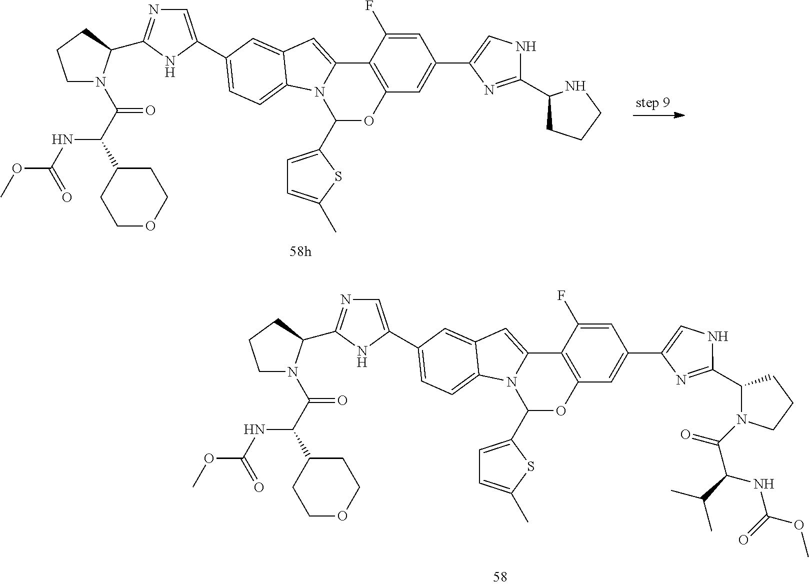

### Step 1

To a solution of core 4 (10 g, 29 mmol) and compound 58a (4.4 g, 35 mmol) in MeCN (50 mL) was added TFA (990 mg, 8.7 mmol), and the mixture was allowed to stir at 25° C. for 1 h, appearing mass of solid. Filtered the solid, washed with MeCN, dried it and gained the desired compound 58b as a white solid (11 g, 85% yield).

### Step 2

To the solution of compound 58b (11 g, 24.4 mmol) in dry toluene (100 mL) was added DDQ (8.3 g, 36.6 mmol). After refluxing for 2 hours under N₂ atmosphere, the solvent was removed in vacuo and diluted with EtOAc. The organic layer was washed with saturated Na₂S₂O₃ aqueous and brine, dried over Na₂SO₄, filtered and concentrated in vacuo. The residue obtained was washed with MeOH (20 mL), filtered and the solid just was the desired compound 58c (10 g, 92% yield). LC/MS: Anal. Calcd. For [M+H]⁺ C20H12BrClFNOS; 449.95. found 450.

### Step 3

Compound 58c-1 was got from compound 58c (5.9 g) by SFC separation by using the following conditions.

Column: Chiralpak AS-H 150*4.6 mm I.D., 5 um

Mobile phase: methanol (0.05% DEA) in CO₂ from 5% to 40%

Flow rate: 3 mL/min

Wavelength: 220 nm

Compound 58c-1 (2.9 g, 49% yield). LC/MS: Anal. Calcd. For [M+H]⁺ C20H12BrClF NOS; 449.95. found 450.

### Step 4

A suspension of the compound 58c-1 (5.9 g, 13.1 mmol), bis(pinacolato)diboron (4 g, 15.8 mmol), KOAc (2.6 g, 26.2 mmol) and Pd(dppf)Cl₂ (0.48 g, 0.655 mmol) in dioxane (120 mL) was allowed to stir at 100° C. under N₂ atmosphere for 2 hours. The reaction mixture was cooled and concentrated in vacuo, then chromatographed on silica gel (eluent:petroleum ether/EtOAc from 100:1 to 20:1) to provide compound 58d (6 g, 92%). LC/MS: Anal. Calcd. For [M+H]⁺ C26H24BClFNO3S; 496.12. found 496.2.

### Step 5

A suspension of the compound 58d (3.5 g, 7.06 mmol), compound cap 32 (3.2 g, 7.77 mmol), Na₂CO₃ (1.5 g, 14.12 mmol) and Pd(dppf)Cl₂ (258 mg, 0.35 mmol) in THF/H₂O (36 mL, 5:1) was allowed to stir at 100° C. under N₂ atmosphere for about 15 hours. LC/MS and TLC were detected the reaction. Separated the water phase through the reparatory funnel, and the organic phase was concentrated in vacuo and purified using Flash column chromatography on silica gel (eluent:from petroleum ether/ethyl acetate=1/5 to DCM/MeOH 50/1) to provide the desired compound 58e (4.5 g, 90.6% yield). LC/MS Anal. Calcd. For [M+H]⁺ C36H35ClFN5O5S; 704.20. found 704.2.

### Step 6

To a mixture of compound 58e (1 g, 1.42 mmol), bis(pinacolato)diboron (397 mg, 1.56 mmol), KOAc (278 mg, 2.84 mmol), Pd₂(dba)₃ (130 mg, 0.14 mmol), X-Phos (135 mg, 0.284 mmol) degassed and sealed under N₂ was added dry dioxane (12 mL), following further N₂ purging. The mixture was allowed to stir at 100° C. for about 15 hours. Under standard work-up to provide the residue which was purified using Flash column chromatography on silica gel (eluent: DCM/MeOH from 100:1 to 50:1) to provide the compound 58f (0.79 g, 70% yield). LC/MS Anal. Calcd. For [M+H]⁺ C42H47BFN5O7S; 796.33. found 796.4.

### Step 7

A suspension of the compound 58f (3.5 g, 4.4 mmol), cap 31a (1.7 g, 5.28 mmol), Na₂CO₃ (933 mg, 8.8 mmol) and Pd(dppf)Cl₂ (161 mg, 0.22 mmol) in THF/H₂O (48 mL, 5:1) was allowed to stir at 100° C. under N₂ atmosphere for about 15 hours. LCMS and TLC were detected the reaction. Separated the water phase through the reparatory funnel, and the organic phase was concentrated in vacuo and purified using Flash column chromatography on silica gel (eluent:DCM/MeOH from 100:1 to 50:1) to provide the compound 58g (2.1 g, 53%). LC/MS Anal. Calcd. For [M+H]⁺ C48H53FN8O7S; 905.37. found 905.5.

### Step 8

To a solution of compound 58g (2.1 g, 2.32 mmol) in dry dioxane (8 mL) was added HCl-dioxane (6 mL) through syringe and stirred at 25° C. for 2 hours, then concentrated in vacuo and dried under high vacuum to provide HCl salt of compound 58h (1.87 g, 98%). LC/MS Anal. Calcd. For [M+H]⁺ C43H45FN8O5S: 805.32. found 805.4.

### Step 9

To a mixture of the compound 58h (500 mg, 0.62 mmol), cap 31c (108.5 mg, 0.62 mmol) and DIPEA (160 mg, 1.24 mmol) in DMF (3 mL) was added HATU (235.6 mg, 0.62 mmol). The resulting mixture was allowed to stir at 25° C. for 2 hours before the solution was transferred to purification team to do HPLC purification to provide the desired compound 58 (340 mg, 57%). ¹H NMR (MeOD) δ: 7.98 (s, 1H), 7.95 (s, 1H), 7.59-7.75 (m, 2H), 7.15-7.45 (m, 4H), 6.94-7.12 (m, 1H), 6.46 (d, J=5.48 Hz, 2H), 5.17-5.34 (m, 2H), 4.32 (s, 1H), 4.22 (s, 1H), 3.82-4.14 (m, 5H), 3.64 (s, 6H), 3.35-3.50 (m, 2H) 2.55 (d, J=5.48 Hz, 2H), 2.02-2.37 (m, 11H), 1.33-1.63 (m, 4H), 0.81-1.01 (m, 6H). LC/MS Anal. Calcd. For [M+H]⁺ C50H56FN9O8S; 962.40. found 962.5.

### Example 46

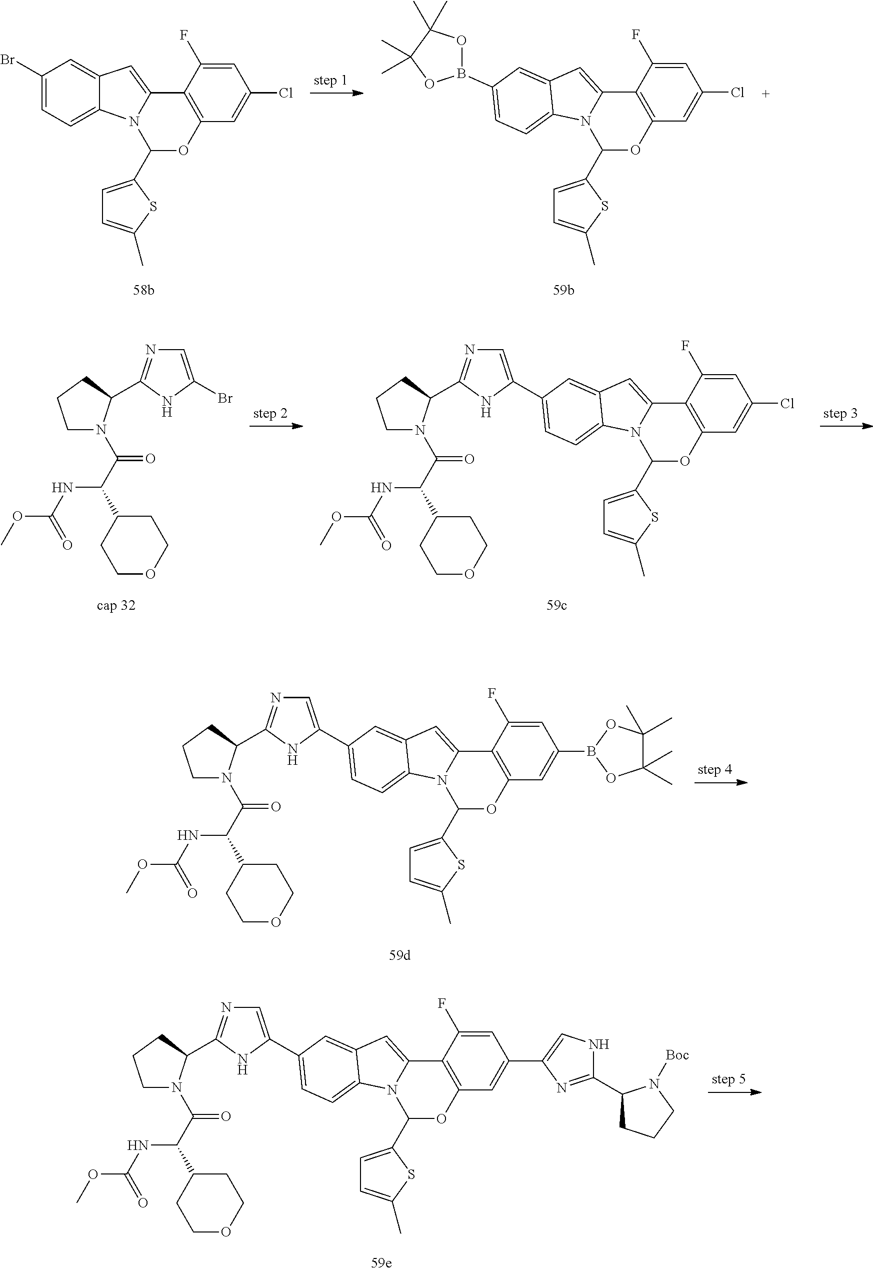

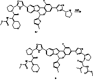

### Step 1

Compound 58b was made from Example 45. A suspension of the compound 58b (6.1 g, 13.6 mmol), bis(pinacolato)diboron (4.15 g, 16.3 mmol), KOAc (2.7 g, 27.2 mmol) and Pd(dppf)Cl₂ (0.49 g, 0.68 mmol) in dioxane (120 mL) was allowed to stir at 100° C. under N₂ atmosphere for 2 hours. The reaction mixture was cooled and concentrated in vacuo, then chromatographed on silica gel (eluent: petroleum ether/ethyl acetate from 100:1 to 20:1) to provide compound 59b (6.2 g, 92%). LC/MS Anal. Calcd. For [M+H]⁺ C26H24BClFNO3S; 496.12. found 496.2.

### Step 2

A suspension of the compound 59b (3.5 g, 7.06 mmol), cap 32 (3.2 g, 7.77 mmol), Na₂CO₃ (1.5 g, 14.12 mmol) and Pd(dppf)Cl₂ (258 mg, 0.35 mmol) in THF/H₂O (36 mL, 5:1) was allowed to stir at 100° C. under N₂ atmosphere for about 15 hours. LCMS and TLC were detected the reaction. Separated the water phase through the separatory funnel, and the organic phase was concentrated in vacuo and purified using Flash column chromatography on silica gel (eluent:from petroleum ether/ethyl acetate=1:5 to DCM/MeOH=50:1) to provide the compound 59c (4.5 g, 90.6% yield). LC/MS Anal. Calcd. For [M+H]⁺ C36H35ClFN5O5S; 704.20. found 704.3.

### Step 3

To a mixture of compound 59c (2 g, 2.84 mmol), bis(pinacolato)diboron (867 mg, 3.4 mmol), KOAc (557 mg, 5.68 mmol), Pd₂(dba)₃ (260 mg, 0.28 mmol), X-Phos (270 mg, 0.56 mmol) degassed and sealed under N₂ was added dry dioxane (12 mL), following further N₂ purging. The mixture was allowed to stir at 100° C. for about 15 hours. Under standard work-up to provide the residue which was purified using Flash column chromatography on silica gel (eluent: from DCM to DCM/MeOH 50:1) to provide the compound 59d (1.78 g, 79% yield). LC/MS Anal. Calcd. For [M+H]⁺ C42H47BFN5O7S; 796.33. found 796.4.

### Step 4

A suspension of the compound 59d (4 g, 5.03 mmol), cap 31a (1.9 g, 6.03 mmol), Na₂CO₃ (1.07 g, 10.1 mmol) and Pd(dppf)Cl₂ (184 mg, 0.25 mmol) in THF/H₂O (48 mL, 5:1) was allowed to stir at 100° C. under N₂ atmosphere for about 15 hours. LCMS and TLC were detected the reaction. Separated the water phase through the separatory funnel, and the organic phase was concentrated in vacuo and purified using Flash column chromatography on silica gel (eluent:DCM/MeOH from 100:1 to 30:1) to provide the compound 59e (2.2 g, 49% yield). LC/MS Anal. Calcd. For [M+H]⁺ C48H53FN8O7S; 905.37. found 905.5.

### Step 5

To a solution of compound 59e (2.2 g, 2.43 mmol) in dry dioxane (8 mL) was added HCl-dioxane (6 mL) through syringe and stirred at 25° C. for 2 hours, then concentrated in vacuo and dried under high vacuum to provide the HCl salt of compound 59f (1.96 g, 98% yield). LC/MS Anal. Calcd. For [M+H]⁺ C43H45FN8O5S; 804.32. found 805.4.

### Step 6

To a mixture of the compound 59f (500 mg, 0.62 mmol), cap 31c (108.5 mg, 0.62 mmol) and DIPEA (160 mg, 1.24 mmol) in DMF (3 mL) was added HATU (235.6 mg, 0.62 mmol). The resulting mixture was allowed to stir at 25° C. for 2 hours before the solution was transferred to purification team to do HPLC purification to provide the desired compound 59 (310 mg, 52% yield). ¹H NMR (MeOD) δ: 7.88 (s, 1H), 7.82 (s, 1H), 7.63-7.65 (m, 2H), 7.15-7.45 (m, 4H), 6.94-7.12 (m, 1H), 6.46 (d, J=5.48 Hz, 2H), 5.17-5.34 (m, 2H), 4.32 (s, 1H), 4.22 (s, 1H), 3.82-4.14 (m, 5H), 3.64 (s, 6H), 3.35-3.50 (m, 2H) 2.55 (d, J=5.48 Hz, 2H), 2.02-2.37 (m, 11H), 1.33-1.63 (m, 4H), 0.79-1.01 (m, 6H). LC/MS Anal. Calcd. For [M+H]⁺ C50H56FN9O8S; 961.40. found 962.5.

### Example 47

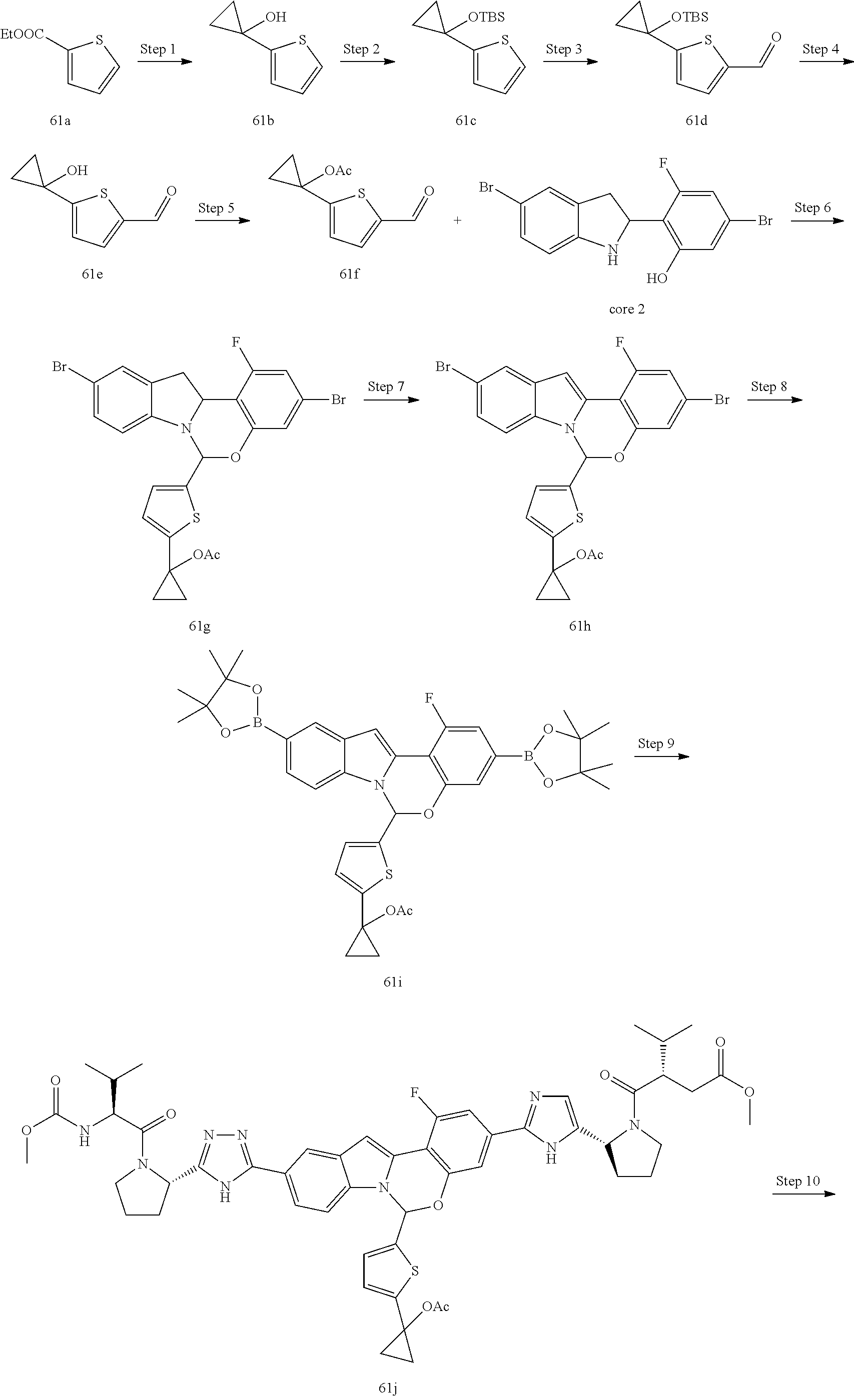

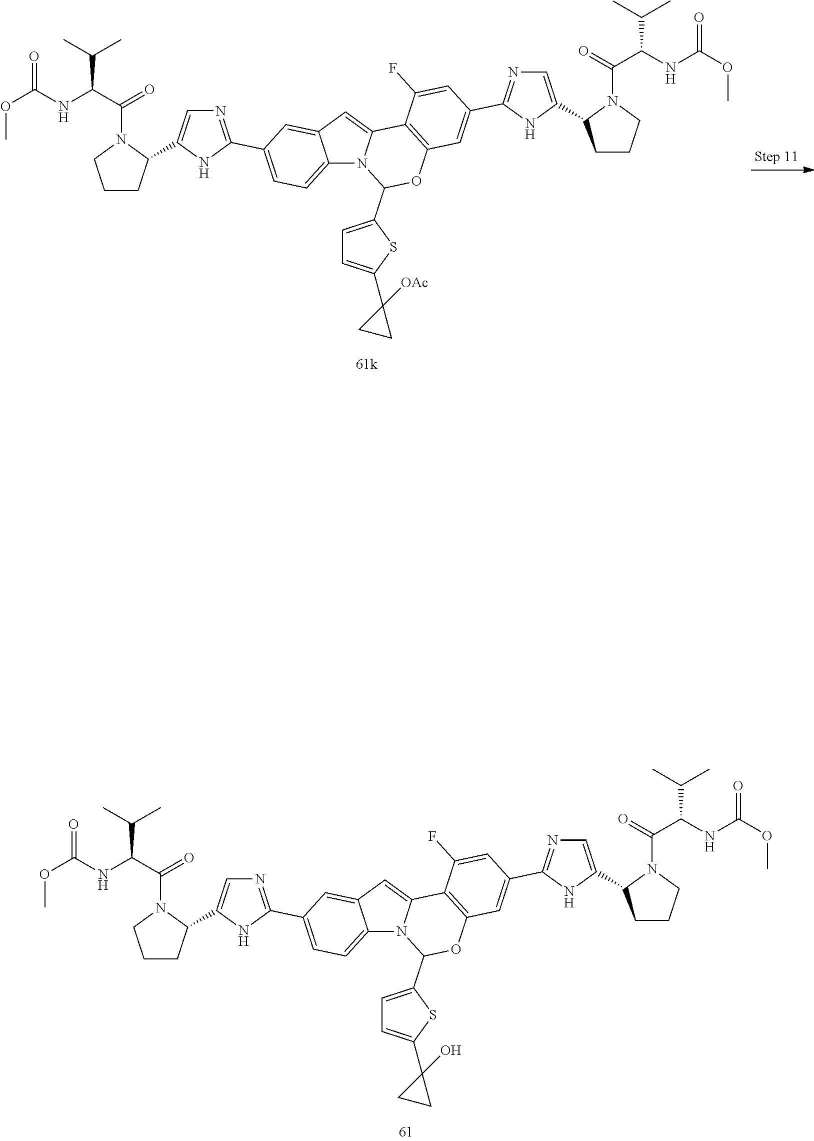

### Step 1

To a solution of 61a (6 g, 40 mmol) in THF (60 mL) was added Titanium isopropoxide (2.3 g, 8 mmol) at 0° C. with stirring. After stirring for 10 minutes, to the mixture was added Ethylmagnesium bromide (30 mL, 3M in Et₂O, 90 mmol) dropwise at 10˜15° C. The solution was allowed to stir at 25° C. for 30 minutes before quenched with 6 mL of water. After filtration and concentration, the residue obtained was purified using Flash column chromatography on silica gel (50 g, Hexane/EtOAc 0% to 20%) to provide 61b (2.2 g, 34% yield).

### Step 2

To a solution of 61b (2.2 g, 15.7 mmol) in dichloromethane (15 mL) was added 2,6-lutidine (2.5 g, 23.6 mmol), TBSOTf (4.2 g, 15.7 mmol) at 25° C. After stirring at 25° C. for 30 minutes, the reaction mixture was poured into water, extracted with ethyl acetate. The combined organic phases were dried over Na₂SO₄. After filtration and concentration, the residue obtained was purified using Flash column chromatography on silica gel (20 g, Hexane/EtOAc 0% to 5%) to provide 61c (2.2 g, 55% yield).

### Step 3

To a solution of 61c (2.2 g, 8.7 mmol) in THF (20 mL) was added LDA (6.5 mL, 2 M in THF, 13 mmol) at −78° C. with stirring. After stirring for 30 minutes, to the mixture was added DMF (1.2 g, 17.4 mmol). The solution was allowed to stir at −78° C. for another 30 minutes, before quenched with aqueous NH₄Cl, and extracted with ethyl acetate. The combined organic layers were washed with brine, dried over Na₂SO₄. After filtration and concentration, the residue obtained was purified using Flash column chromatography on silica gel (20 g, Hexane/EtOAc 0% to 10%) to provide 61d (1.4 g, 58% yield).

### Step 4

To a solution of 61d (1.4 g, 5 mmol) in methanol (5 mL) was added 4 N HCl in methanol (5 mL) at 25° C. After stirring for 30 minutes, the mixture was concentrated in vacuo. The residue obtained was neutralized by 1N sodium bicarbonate and extracted with ethyl acetate. The combined organic layers were washed with brine, dried over Na₂SO₄. After filtration and concentration, the residue obtained was purified using Flash column chromatography on silica gel (20 g, Hexane/EtOAc 0% to 25%) to provide 61e (0.8 g, 95% yield).

### Step 5

To a solution of 61e (840 mg, 5 mmol) in dichloromethane (10 mL) was added triethylamine (1 g, 10 mmol), acetyl chloride (600 mg, 7.5 mmol) at 0° C. After stirring at 25° C. for 30 minutes, the reaction mixture was poured into water, extracted with ethyl acetate. The combined organic layers were dried over Na₂SO₄. After filtration and concentration, the residue obtained was purified using Flash column chromatography on silica gel (10 g, Hexane/EtOAc 0% to 10%) to provide 61f (0.8 g, 95% yield).

### Step 6

To a mixture of 61f (800 mg, 4 mmol) and core 2 (1.2 g, 3 mmol) in anhydrous CH₃CN (25 mL) was added TFA (114 mg, 1 mmol) at 25° C. The mixture was allowed to stir at 25° C. for 6 hours before filtrated. The collected solid was washed with CH₃CN to provide 61g (0.8 g, 47% yield).

### Step 7

To a mixture of 61g (800 mg, 1 mmol) in dry toluene (20 mL) was added DDQ (470 mg, 2.1 mmol). After refluxing for 2 hours, the solvent was removed in vacuo and diluted with EtOAc. The organic layer was washed with saturated aqueous Na₂S₂O₃ and brine, dried over Na₂SO₄. After filtration and concentration, the residue obtained was purified using Flash column chromatography on silica gel (10 g, Hexane/EtOAc 0% to 10%) to provide 61h (0.65 g, 81% yield).

### Step 8

To a solution of 61h (650 mg, 1.1 mmol) in dioxane was added BIS(PINACOLATO)DIBORON (710 mg, 2.8 mmol), Pd(dppf)Cl₂ (160 mg, 0.22 mmol) and KOAc (440 mg, 4.4 mmol). The reaction mixture was allowed to stir under N₂ at 90° C. for 3 hours. Then the reaction mixture was poured into water, and extracted with ethyl acetate. The organic layer was dried over Na₂SO₄. After filtration and concentration, the residue obtained was purified using Flash column chromatography on silica gel (2 g, Hexane/EtOAc 0% to 10%) to provide 61i (0.56 g, 75% yield).

### Step 9

The mixture of 61i (560 mg, 0.83 mmol), cap 31 (740 mg, 2 mmol), Pd(dppf)Cl₂ (140 mg, 0.2 mmol), Na₂CO₃ (400 mg, 4 mmol) in THF/DMF/H₂O (5/1/1, 14 mL) was refluxed for about 15 hours under N₂ atmosphere. After filtration, the filtrate was washed with water (50 mL) and extracted with ethyl acetate (100 mL). The organic layer was washed with brine and dried over Na₂SO₄. After filtration and concentration, the residue obtained was purified using Flash column chromatography on silica gel (2 g, Hexane/EtOAc 20% to 50%) to provide 61j (0.4 g, 48% yield). LC/MS: Anal. Calcd. For [M+H]⁺ C52H58FN9O9S; 1004.41. found 1004.8.

### Step 10

The compound of 61j (0.4 g) was separated by SFC by using the following conditions to provide 61k (0.13 g, 33%).

Column: AS 250 mm×20 mm, 20 um

Solvent: 50% IPA (0.05% NH₃—H₂O)/CO₂

Flow rate: 80 mL/min

Wavelength: 220 nm

### Step 11

To a solution of 61k (130 mg, 0.13 mmol) in methanol (10 mL) was added LiOH (20 mg, 0.5 mmol). The mixture was allowed to stir at 25° C. for about 15 hours, and then neutralized with acetic acid. The solution was subjected directly to Pre-HPLC to provide 61 (52 mg, 41.6%). ¹H NMR (MeOD) δ: 8.02 (m, 2H), 7.88 (s, 1H), 7.76 (s, 1H), 7.68-7.50 (m, 3H), 7.40-7.31 (m, 2H), 7.20 (s, 1H), 6.68-6.60 (m, 1H), 5.25-5.13 (m, 2H), 4.25-4.15 (m, 2H), 4.15-4.05 (m, 2H), 3.90-3.81 (m, 2H), 3.63 (s, 6H), 2.86-2.78 (m, 2H), 2.60-2.40 (m, 2H), 2.31-1.95 (m, 8H), 1.10-1.03 (m, 3H), 1.00-0.81 (m, 12H). LC/MS: Anal. Calcd. For [M+H]⁺ C50H56FN9O8S: 962.40. found 962.6.

### Example 48

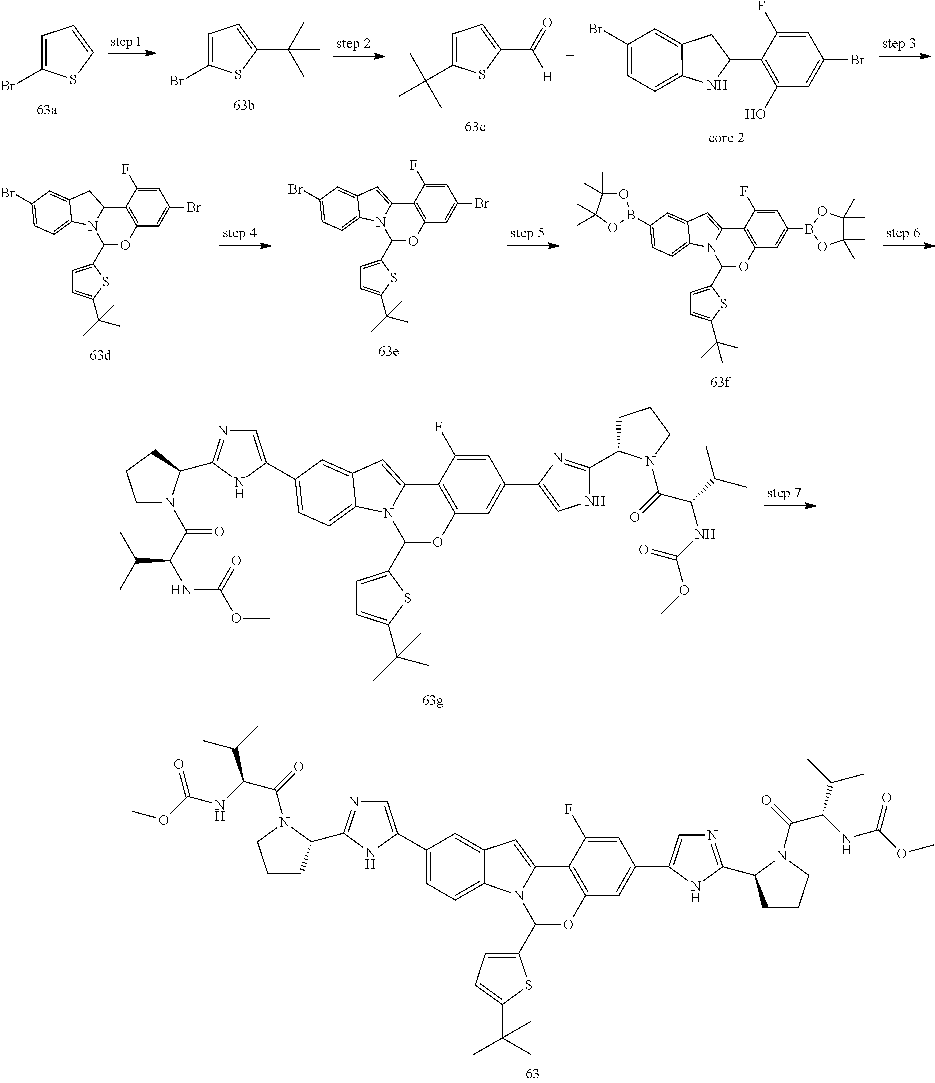

### Step 1

A solution of 63a (1.8 mL, 18 mmol), AlCl₃ (7.35 g, 55 mmol) and t-BuCl (6 mL, 55 mmol) in DCM (20 mL) was allowed to stir at −78° C. for 2 hours under N₂. Then was filtered and the filtrate was concentrated in vacuo and purified using Flash column chromatography on silica gel to provide compound 63b (4 g, yield 82%).

Step 2 To a mixture of 63b (4 g, 18 mmol) in THF (50 mL) was added a 2.5 M solution of n-BuLi (7.2 mL, 18 mmol) at −78° C. under N₂. The mixture was agitated for 1 hour at this temperature then DMF (2.6 g, 36 mmol) was added. The mixture was allowed to stir at 25° C. for 3 hours. Quenched with NH₄Cl saturate solution and extracted with EtOAc. The organic layer was dried over sodium sulfate, and concentrated in vacuo and purified using Flash column chromatography on silica gel to provide compound 63c (2 g, yield 56%). LC/MS: Anal. Calcd. For [M+H]⁺ C9H12OS; 169.06. found 169.10.

### Step 3

To a mixture of 63c (1 g, 3.8 mmol) and core 2 (1 g, 2.6 mmol) in anhydrous CH₃CN (20 mL) was added TFA (0.089 g, 0.78 mmol) at 25° C. The mixture was agitated for 6 hours at 25° C. The reaction mixture became a clear solution and then a solid appeared. The solid was collected by filtration and washed with CH₃CN to provide the desired compound 63d. LC/MS: Anal. Calcd. For [M+H]⁺ C23H20Br2FNOS; 536.96. found 537.60.

### Step 4

The solution of 63d (1 g, 1.9 mmol) in dry toluene (20 mL) was added DDQ (0.634 g, 2.8 mmol). After refluxing for 2 hours, the solvent was removed in vacuo and diluted with EtOAc. The organic layer was washed with saturated Na₂SO₃ aqueous and brine, dried over Na₂SO₄, filtered and concentrated in vacuo. The residue obtained was washed with MeOH (10 mL), filtered and the solid just was the product 63e. LC/MS: Anal. Calcd. For [M+H]⁺ C23H18Br2FNOS; 534.94. found 535.60.

### Step 5

To a solution of 63e (0.8 g, 1.5 mmol) in dioxane was added bis pinacol borate (0.762 g, 3 mmol) and Pd(dppf)Cl₂ (0.11 g, 0.15 mmol) and KOAc (0.764 g, 7.8 mmol). The reaction mixture was allowed to stir under N₂ and heated to 110° C. for about 15 hours. After that, the solvent was removed in vacuo, and the residue obtained was purified using Flash column chromatography on silica gel with silica gel to provide the product 63f. LC/MS: Anal. Calcd. For [M+H]⁺ C35H42B2FNO5S; 630.30. found 630.50.

### Step 6

A suspension of 63f (900 mg, 1.4 mmol), cap 31 (1 g, 2.8 mmol), Pd(dppf)Cl₂ (73 mg, 0.1 mmol), Na₂CO₃ (0.594 g, 5.6 mmol) and in THF/H₂O (10:1, 34 mL) was refluxed at 95° C. for about 15 hours under N₂ atmosphere. After that, the mixture was filtered; the filtrate was washed with water (50 mL) and extracted with EtOAc (100 mL), washed with brine and dried over anhydrous sodium sulfate. After being concentrated in vacuo, the residue obtained was transferred to purification team to do HPLC purification to provide 63g (600 mg. 43.6% yield).

### Step 7

Compound 63 was separated from compound 63g (600 mg) by SFC by using the following condition:

Column: Chiral OZ 150×4.6 mm I.D.

Mobile phase: 50% ethanol (0.05% DEA) in CO₂

Flow rate: 2.0 mL/min

Wavelength: 220 nm

Compound 63 (120 mg, 41%). ¹H NMR (MeOD) δ: 8.0 (s, 1H), 7.8 (s, 1H), 7.8 (s, 1H), 7.7 (s, 1H), 7.5-7.4 (m, 2H), 7.3-7.3 (d, J=11.2 Hz, 1H), 7.3 (s, 1H), 7.1 (m, 1H), 6.6 (s, 1H), 6.5 (s, 1H), 5.2-5.1 (m, 2H), 4.2-4.1 (m, 2H), 4.1-4.0 (m, 2H), 3.9-3.7 (m, 2H), 3.6 (m, 6H), 2.6-2.4 (m, 2H), 2.3-2.2 (m, 2H), 2.1 (m, 4H), 2.0-1.9 (m, 2H), 1.2 (s, 9H), 0.9-0.8 (m, 12H). LC/MS: Anal. Calcd. For [M+H]⁺ C51H60FN9O7S; 962.43. found 962.01.

### Example 49

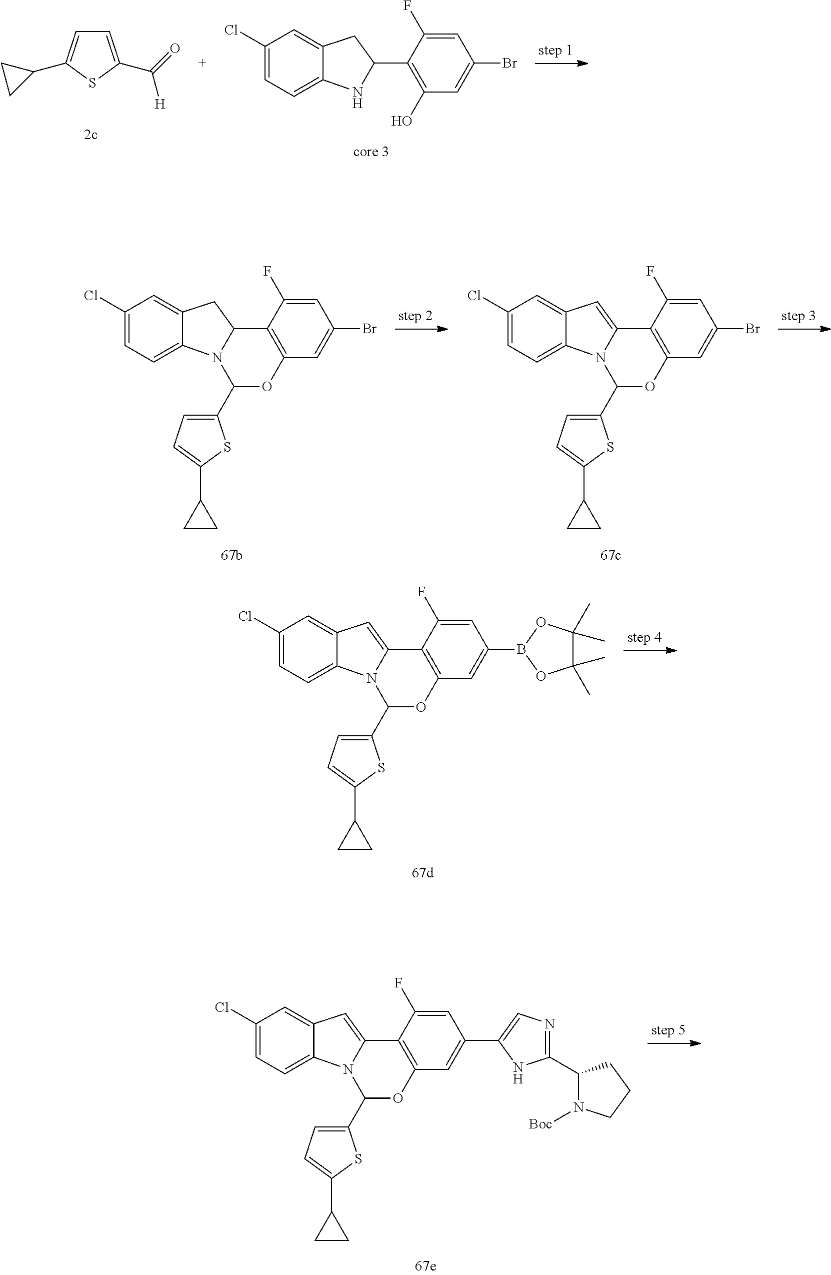 [SMILES: O=Cc1ccc(C2CC2)s1 ; Oc1cc(Br)cc(F)c1C1Cc2cc(Cl)ccc2N1 ; Fc1cc(Br)cc2c1C1Cc3cc(Cl)ccc3N1C(c1ccc(C3CC3)s1)O2 ; Fc1cc(Br)cc2c1-c1cc3cc(Cl)ccc3n1C(c1ccc(C3CC3)s1)O2 ; CC1(C)OB(c2cc(F)c3c(c2)OC(c2ccc(C4CC4)s2)n2c-3cc3cc(Cl)ccc32)OC1(C)C ; CC(C)(C)OC(=O)N1CCC[C@H]1c1ncc(-c2cc(F)c3c(c2)OC(c2ccc(C4CC4)s2)n2c-3cc3cc(Cl)ccc32)[nH]1]

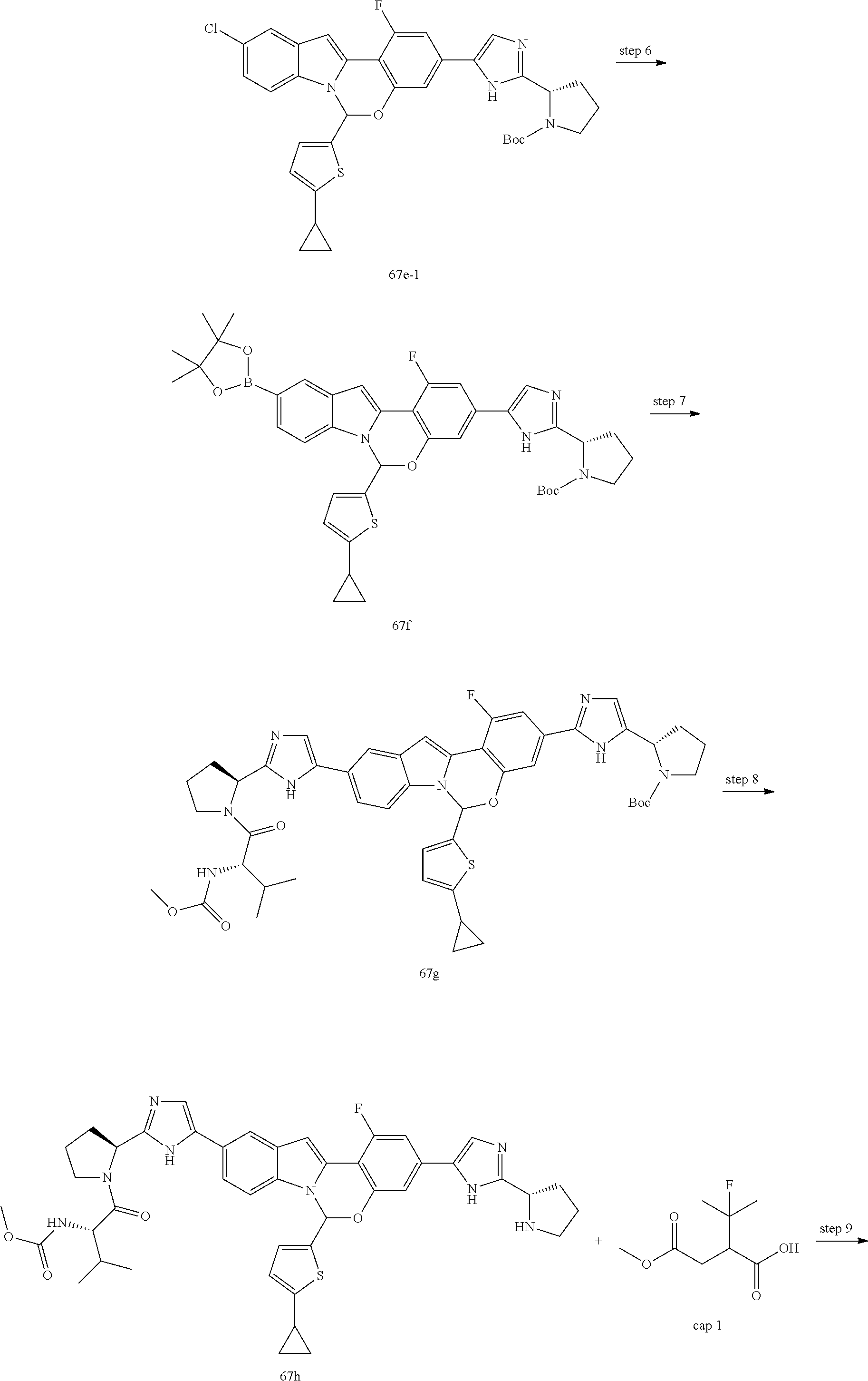

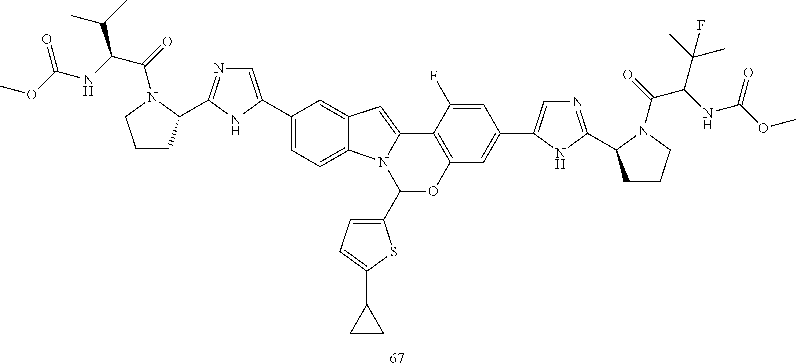

### Step 1

Compound 2c was made from Example 33. To a mixture of core 3 (20 g, 58 mmol) and compound 2c (15 g, 99 mmol) in anhydrous CH₃CN (500 mL) was added TFA (1.98 g, 17.4 mmol) at 25° C. The mixture was agitated for 6 hours at 25° C. The reaction mixture became a clear solution and then a solid appeared. The solid was collected by filtration and washed with CH₃CN to provide compound 67b (25 g, 86.6% yield). LC/MS: Anal. Calcd. For [M+H]⁺ C22H16BrClFNOS; 477.98. found 477.8.

### Step 2

The solution of compound 67b (25 g, 53 mmol) in dry toluene (250 mL) was added DDQ (18 g, 79.5 mmol). After refluxing for 2 hours, the solvent was removed in vacuo and diluted with EtOAc. The organic layer was washed with saturated Na₂S₂O₃ aqueous and brine, dried over Na₂SO₄, filtered and concentrated in vacuo. The residue obtained was washed with MeOH (10 mL), filtered and the solid just was the compound 67c (20 g, 80.3% yield). LC/MS: Anal. Calcd. For [M+H]⁺ C22H14BrClFNOS; 475.96. found 476.0.

### Step 3

A suspension of the compound 67c (20 g, 0.042 mol), bis(pinacolato)diboron (11.76 g, 46 mmol), KOAc (12.3 g, 12.6 mmol) and Pd(dppf)Cl₂ (3.07 g, 42 mmol) in dioxane (400 mL) was allowed to stir at 100° C. under N₂ atmosphere for about 15 hours. The reaction mixture was cooled and concentrated in vacuo, then chromatographed on silica gel (petroleum ether:ethyl acetate=10:1) to provide the compound 67d (14 g, 63.9% yield). LC/MS: Anal. Calcd. For [M+H]⁺ C28H26BClFNO3S; 522.14. found 522.2.

### Step 4

A suspension of the compound 67d (3 g, 5.8 mmol), cap 31a (2 g, 6.4 mmol), Pd(dppf)Cl₂ (0.42 g, 0.58 mmol), Na₂CO₃ (1.84 g, 17.4 mmol) and in THF/H₂O (5:1, 96 mL) was refluxed at 80° C. for about 15 hours under N₂ atmosphere. After that, the mixture was filtered; the filtrate was washed with water (50 mL) and extracted with EtOAc (100 mL), washed with brine and dried over anhydrous sodium sulfate. After being concentrated in vacuo, the residue obtained was purified using Flash column chromatography on silica gel (petroleum ether:ethyl acetate=1:1) to provide compound 67e (3.05 g, 83.3% yield). LC/MS: Anal. Calcd. For [M+H]⁺ C34H32ClFN4O3S; 631.19. found 631.2.

### Step 5

The compound 67e-1 was separated from compound 67e (3.05 g) by SFC using the following condition:

Column: Chiralcel OJ-3 50*4.6 mm I.D.

Mobile phase: 40% methanol (0.05% DEA) in CO₂

Flow rate: 4.0 mL/min

Wavelength: 220 nm

Compound 67e-1 (1.88 g, 60% yield). LC/MS: Anal. Calcd. For [M+H]⁺ C34H32ClFN4O3S; 631.19. found 631.2.

### Step 6

To a mixture of 67e-1 (1.88 g, 2.98 mmol), bis(pinacolato) diboron (0.98 g, 3.87 mmol), KOAc (0.88 g, 8.94 mmol), Pd₂(dba)₃ (0.27 g, 0.30 mmol), X-Phos (0.88 g, 0.298 mmol) degassed and sealed under N₂ was added dry dioxane. Following further N₂ purging. The mixture was allowed to stir at 100° C. for about 15 hours. Under standard work-up to provide the residue which was purified using Flash column chromatography on silica gel on silica (petroleum ether:ethyl acetate=1:2) to provide compound 67f (1.79 g, 83.3% yield). /MS: Anal. Calcd. For [M+H]⁺ C40H44BFN405S; 723.31. found 723.1.

### Step 7

A suspension of 67f (0.4 g, 0.55 mmol), cap 31 (0.23 g, 0.61 mmol), Pd(dppf)Cl₂ (0.04 g, 0.055 mmol), Na₂CO₃ (0.17 g, 1.65 mmol) and in THF/H₂O (5:1, 24 mL) was refluxed at 80° C. for about 15 hours under N₂ atmosphere. After that, the mixture was filtered; the filtrate was washed with water (50 mL) and extracted with EtOAc (100 mL), washed with brine and dried over anhydrous sodium sulfate. After being concentrated in vacuo, the residue obtained was purified using Flash column chromatography on silica gel (petroleum ether:ethyl acetate=10:1) to provide compound 67g (0.35 g, 71.4% yield).

### Step 8

The compound 67g (0.35 g, 0.39 mmol) was added into HCl/CH₃OH (20 mL). Then the mixture was allowed to stir at 25° C. for 2-3 hours. When the reaction completed, the mixture was concentrated in vacuo to provide the crude 67h (0.31 g, 99.7% yield).

### Step 9

To a mixture of the crude 67h (0.12 g, 0.13 mmol), cap 1 (0.028 g, 0.15 mmol) and DIPEA (0.2 mL) in DMF (3 mL) was added HATU (0.058 g, 0.15 mmol). The resulting mixture was allowed to stir at 25° C. The mixture was purified using Pre-HPLC to provide compound 67 (65.3 mg, 52.1% yield). ¹H NMR (MeOD) δ: 7.99 (s, 1H), 7.90 (m, 1H), 7.74-7.77 (m, 2H), 7.54 (m, 1H), 7.52 (m, 1H), 7.46-7.48 (m, 1H), 7.27-7.38 (m, 1H), 7.16-7.17 (s, 1H), 6.45-6.50 (m, 2H), 5.19-5.26 (m, 2H), 4.70-4.72 (m, 1H), 4.22-4.24 (m, 1H), 4.01 (m, 1H), 3.86-3.93 (m, 3H), 3.67 (m, 6H), 3.43-3.46 (m, 1H), 2.54-2.56 (m, 2H), 2.23-2.27 (m, 2H), 2.16-2.17 (m, 4H), 2.05-2.08 (m, 1H), 1.91-1.95 (m, 1H), 1.41-1.47 (m, 4H), 1.24-1.29 (m, 2H), 0.89-0.94 (m, 9H), 0.51-0.55 (m, 2H). LC/MS: Anal. Calcd. For [M+H]⁺ C50H55F2N9O7S; 963.39. found 964.

The following compounds of the present invention were made using the methods described in the Example above and substituting the appropriate reactants or reagents.

| | | | Observed |
|---|---|---|---|
| ID | Structure | Isomer | [M + H]+ |
| 29 30 31 32 | 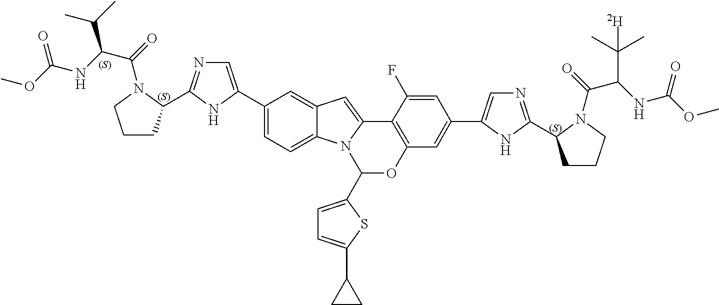 | Isomer 1 Isomer 2 Isomer 3 Isomer 4 | 947.6 947.6 947.8 947.6 |
| 68 69 70 | 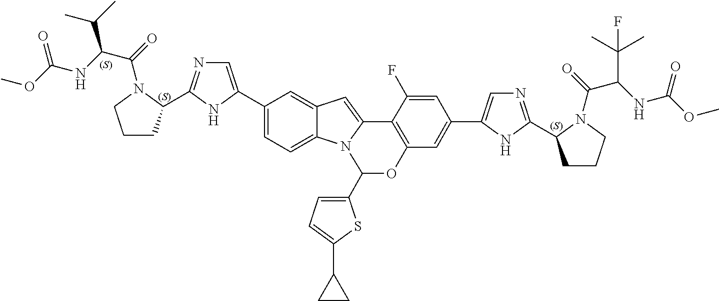 | Isomer 2 Isomer 3 Isomer 4 | 964.8 964.6 964.6 |
| 79 80 | 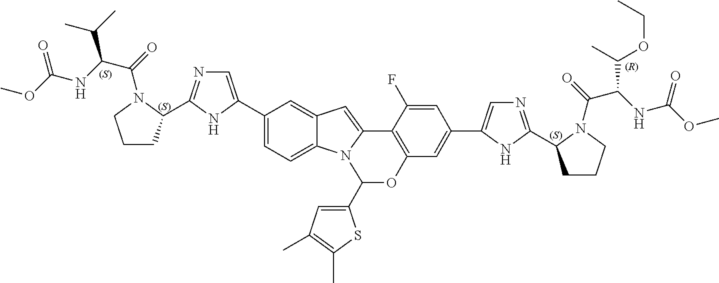 | Isomer 1 Isomer 2 | 964.6 964.4 |
| 98 99 100 | 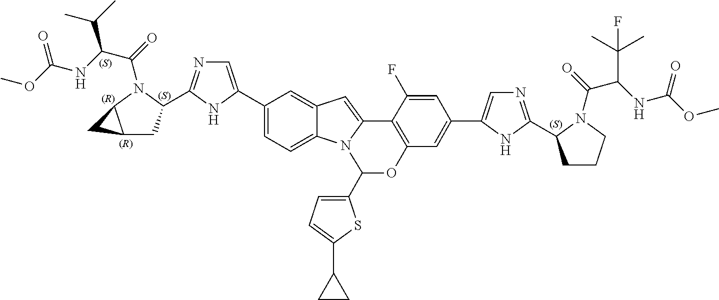 | Isomer 2 Isomer 3 Isomer 4 | 976.4 976.4 976.6 |
| 101 102 | 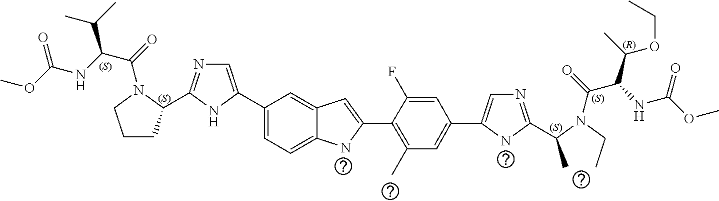 | Isomer 1 Isomer 2 | 976.6 976.5 |
| 106 108 | 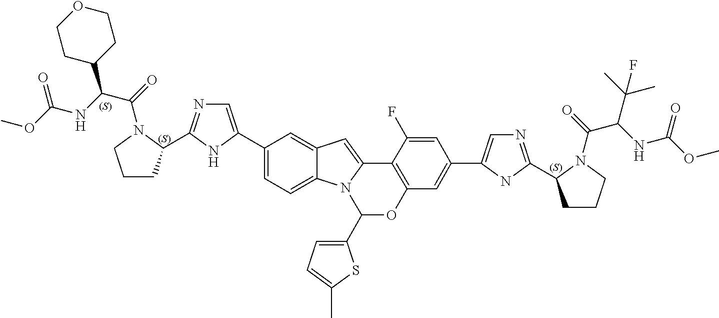 | Isomer 2 Isomer 4 | 980.6 980.4 |
| 122 123 | 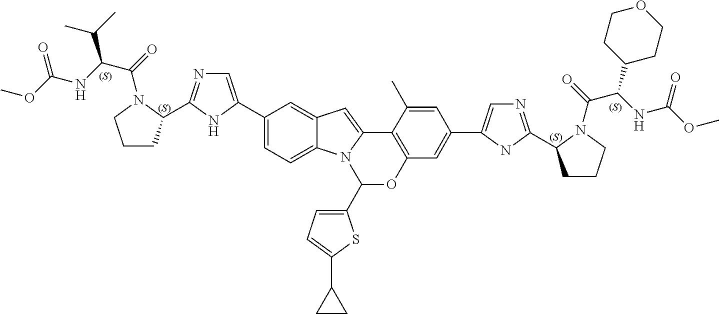 | Isomer 1 Isomer 2 | 984.6 984.6 |
| 154 155 156 157 | 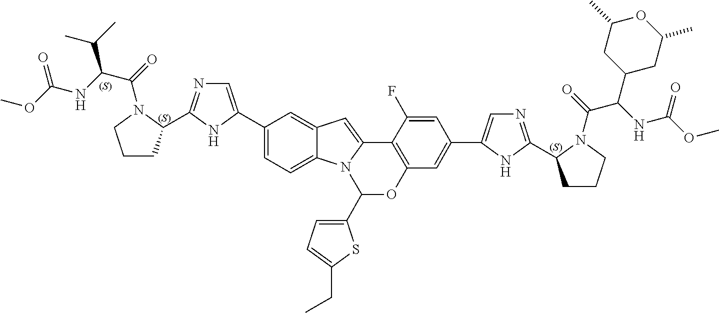 | Isomer 1 Isomer 2 Isomer 3 Isomer 4 | 1004.6 1004.4 1004.6 1004.4 |
| 158 159 160 161 162 163 164 165 | 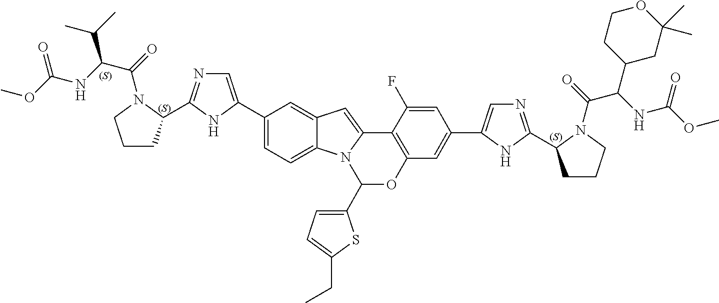 | Isomer 1 Isomer 2 Isomer 3 Isomer 4 Isomer 5 Isomer 6 Isomer 7 Isomer 8 | 1004.4 1004.6 1004.5 1004.4 1004.6 1004.6 1004.4 1004.4 |
| 169 171 | 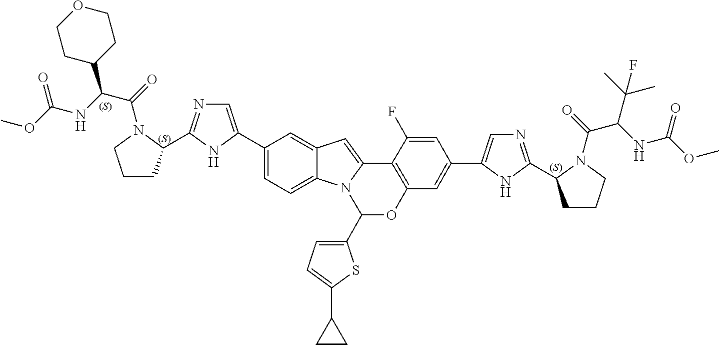 | Isomer 2 Isomer 4 | 1006.6 1006.6 |
| 186 187 189 | 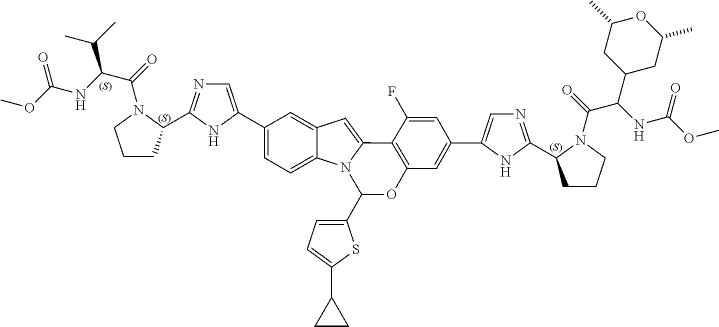 | Isomer 1 Isomer 2 Isomer 4 | 1016.4 1016.6 1016.5 |
| 210 211 212 213 214 215 216 217 | 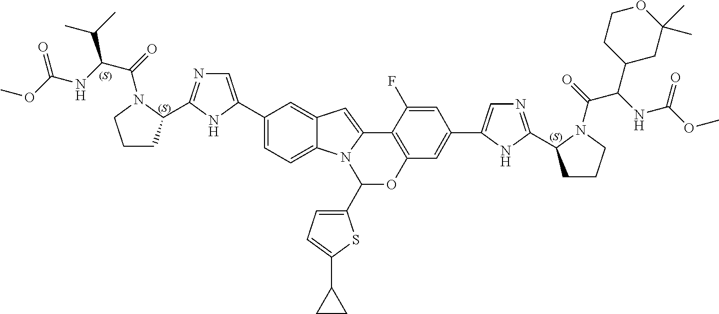 | Isomer 1 Isomer 2 Isomer 3 Isomer 4 Isomer 5 Isomer 6 Isomer 7 Isomer 8 | 1016.6 1016.6 1016.6 1016.4 1016.6 1016.8 1016.6 1016.2 |
| 244 245 | 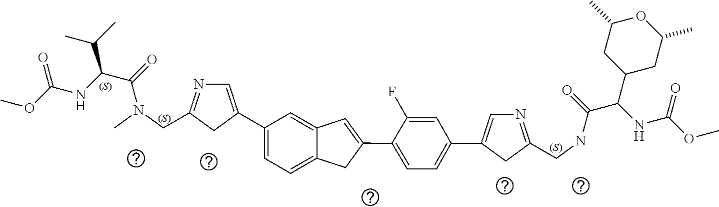 | Isomer 1 Isomer 2 | 1018.6 1018.6 |
| 246 | 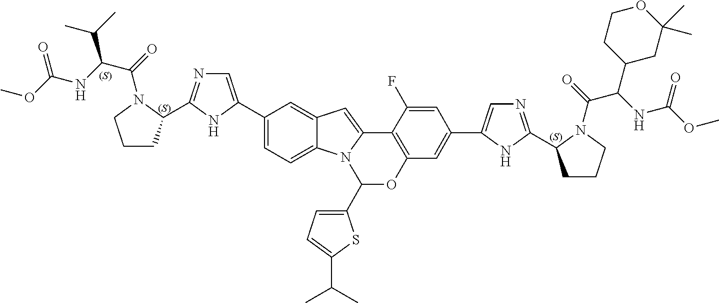 | Isomer 1 | 1018.6 |
| 248 249 251 | 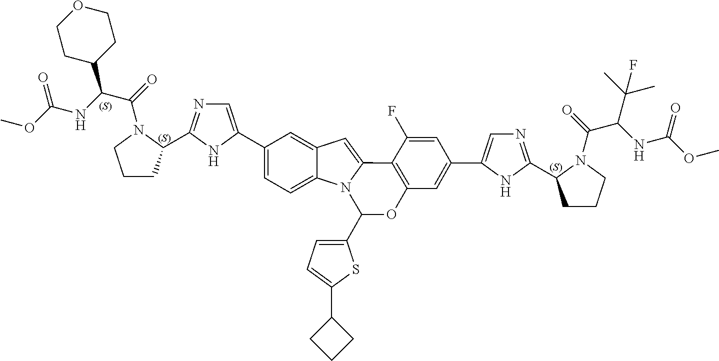 | Isomer 1 Isomer 2 Isomer 4 | 1020.4 1020.6 1020.4 |
| 358 359 | 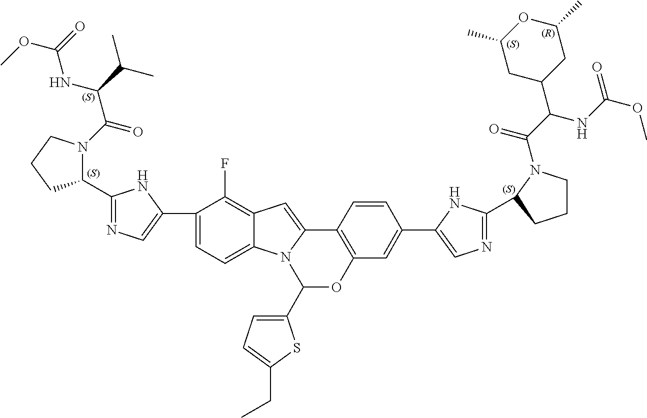 | Isomer 1 Isomer 2 | 1004.4 1004.4 |
| 389 390 | 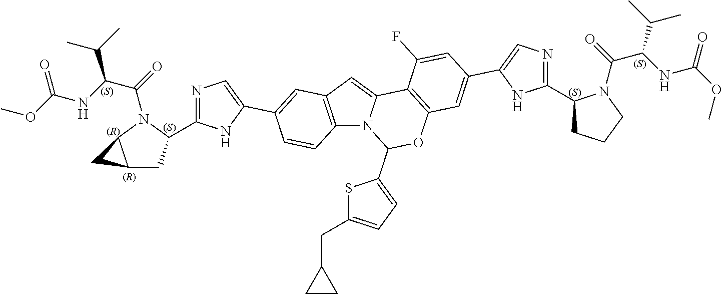 | Isomer 1 Isomer 2 | 972.1 972.1 |
| 709 | 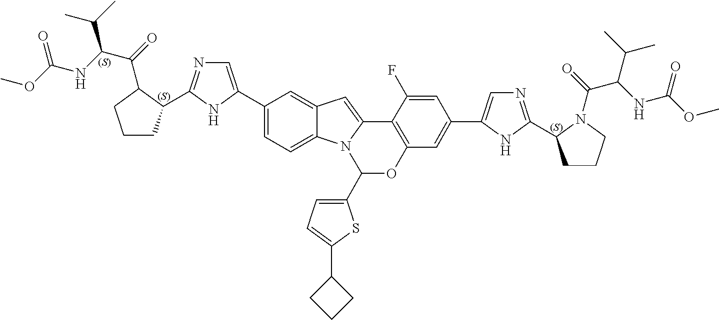 | racemic | 942.4 |

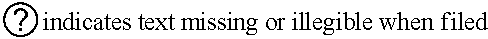

### Example 50

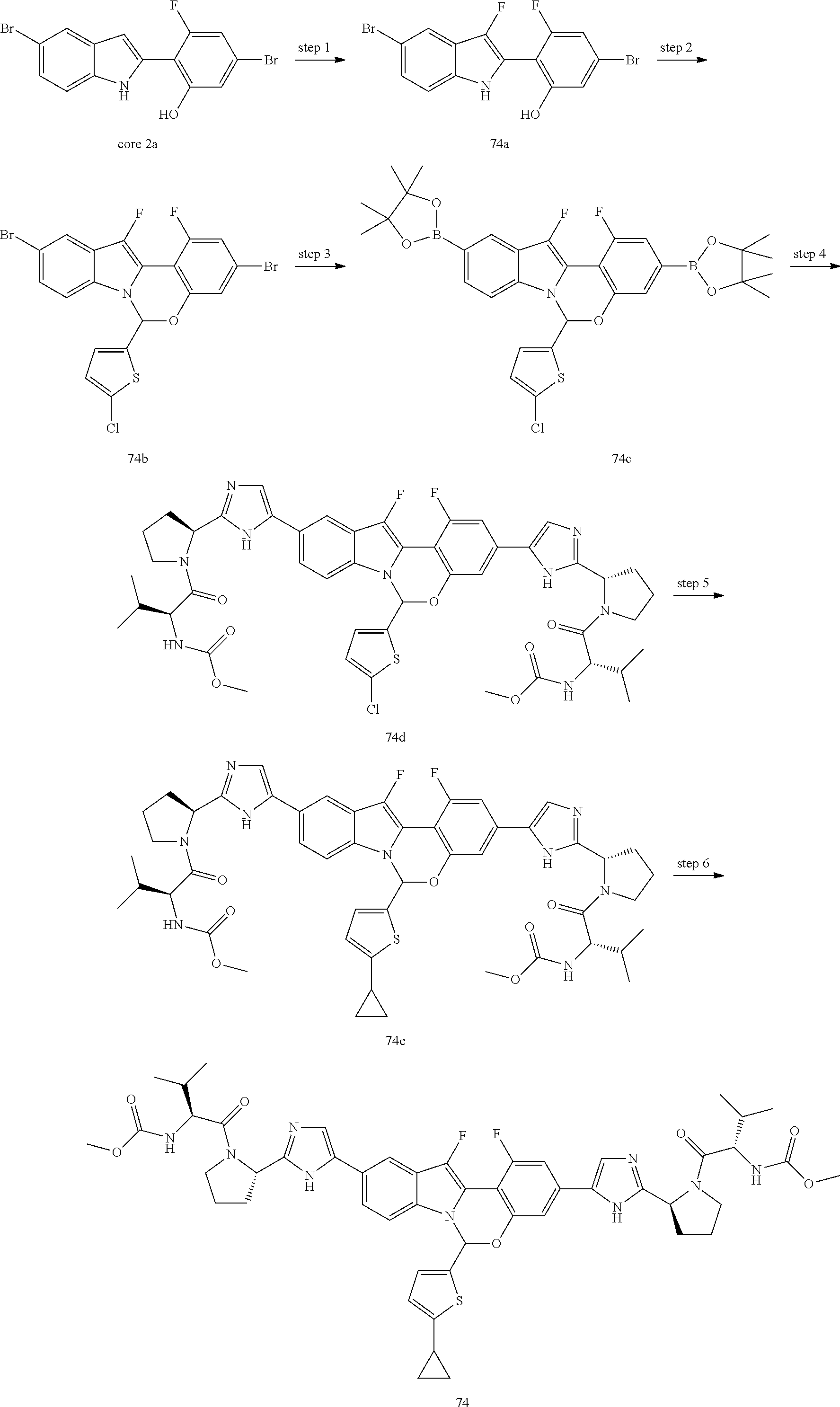

### Step 1

Compound core 2a was prepared in Example 19 of International Publication No. 2012/040923 A1.

To a solution of core 2a (5 g, 13 mmol) in DMSO (30 mL) and MeCN (30 mL) was added selectfluor (3.64 g, 10 mmol) in portions at 0° C. The mixture was allowed to stir at 25° C. for 1 hour. The reaction was diluted with EtOAc, washed with brine, dried over Na₂SO₄ and concentrated under reduced pressure. The crude product was transferred to purification team to do HPLC purification and got 74a (2.5 g, 48%).

### Step 2

One drop of Methanesulfonic acid was added to a solution of 5-chloro-2-thiophenecarboxaldehyde (3.26 g, 22.3 mmol) and compound 74a (3 g, 7.44 mmol) in toluene (15 mL), and the mixture was allowed to stir at 100° C. under N₂ for about 15 hours. The reaction mixture was concentrated in vacuo and purified using Flash column chromatography on silica gel (eluent:petroleum ether:ethyl acetate=10:1) to provide the desired compound 74b (2.5 g, 63% yield).

### Step 3

A suspension of the compound 74b (2.5 g, 4.71 mmol), bis(pinacolato)diboron (2.63 g, 10.3 mmol), KOAc (1.84 g, 18.84 mmol) and Pd(dppf)Cl₂ (0.34 g, 0.47 mmol) in dioxane (25 mL) was allowed to stir at 100° C. under N₂ atmosphere for 2 hours. The reaction mixture was cooled and concentrated in vacuo, then chromatographed on silica gel column (eluent:petroleum ether:ethyl acetate=10:1) to provide compound 74c (2.5 g, 85%). LC/MS Anal. Calcd. For [M+H]⁺ C31H32B2ClF2NO5S; 625.18. found 626.2.

### Step 4

A suspension of the compound 74c (1.5 g, 2.4 mmol), cap 31 (1.89 g, 5.03 mmol), Na₂CO₃ (1.2 g, 9.6 mmol) and Pd(dppf)Cl₂ (175 mg, 0.24 mmol) in THF/H₂O (36 mL, 5:1) was allowed to stir at 100° C. under N₂ atmosphere for about 15 hours. LCMS and TLC were detected the reaction. Separated the water phase through the separatory funnel, and the organic phase was concentrated in vacuo and purified using chromatography on silica gel (eluent:from petroleum ether:ethyl acetate=1:3 to DCM/MeOH=15:1) to provide the desired compound 74d (0.8 g, 35% yield). LC/MS Anal. Calcd. For [M+H]⁺ C47H50ClF2N9O7S; 959.32. found 959.3.

### Step 5

Compound 74d (640 mg, 0.67 mmol), K₂CO₃ (277 mg, 2.01 mmol), Pd₂(dba)₃ (61.3 mg, 0.067 mmol), X-phos (64 mg, 0.134 mmol) and cyclopropylboronicacid (864.3 g, 10.05 mmol) was added to THF/H₂O (12 mL, 5:1), the mixture was allowed to stir at 120° C. in microwave for 2 hours. Cooled the reaction mixture and extracted with EtOAc, filtered the organic phases through a celite pad for several times to remove the residue catalyst, dried over Na₂SO₄, concentrated EtOAc under reduced pressure and transferred to purification team to do HPLC purification to provide 74e (300 mg, 52% yield). LC/MS Anal. Calcd. For [M+H]⁺ C50H55F2N9O7S; 963.39. found 964.5.

### Step 6

The compound 74 was separated from compound 74e (300 mg) by SFC by using the following conditions.

Column: Chiralcel OD-3 150×4.6 mm I.D., 3 um

Mobile phase: 40% of iso-propanol (0.05% DEA) in CO₂

Flow rate: 2.5 mL/min

Wavelength: 220 nm

Compound 74 (120 mg, 40% yield). ¹H NMR (MeOD) δ: 7.99 (s, 1H), 7.91 (s, 1H), 7.71-7.84 (m, 2H), 7.59 (s, 1H), 7.47 (d, T=8.61 Hz, 1H), 7.35-7.42 (m, 1H), 7.27 (s, 1H), 6.52 (s, 2H), 5.24 (s, 2H), 4.19-4.30 (m, 2H), 4.05-4.16 (m, 2H), 3.81-3.93 (m, 2H), 3.65 (s, 6H), 2.48-2.64 (m, 2H), 2.00-2.35 (m, 8H), 1.90-1.98 (m, 1H), 0.85-1.04 (m, 14H), 0.51-0.59 (m, 2H). LC/MS: Anal. Calcd. For [M+H]⁺ C51H59 N9O7S; 963.39. found 964.5.

### Example 51

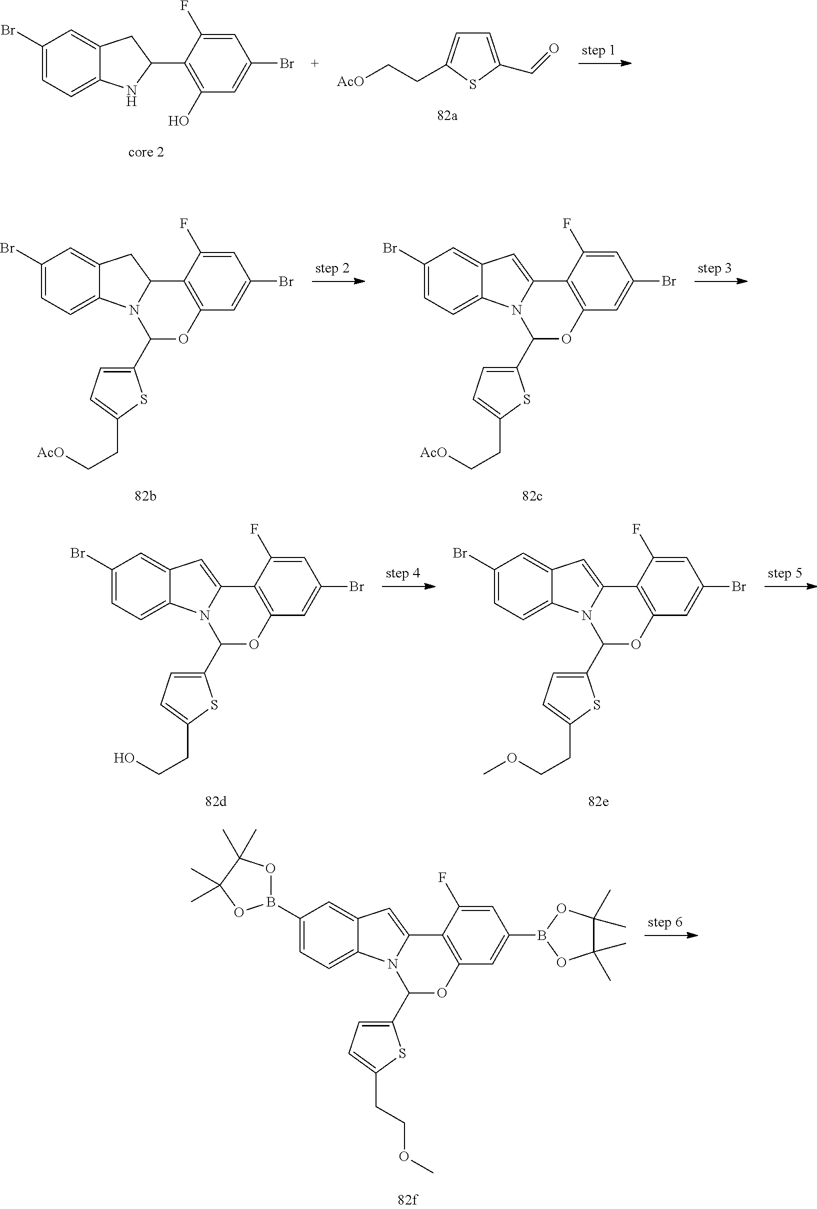

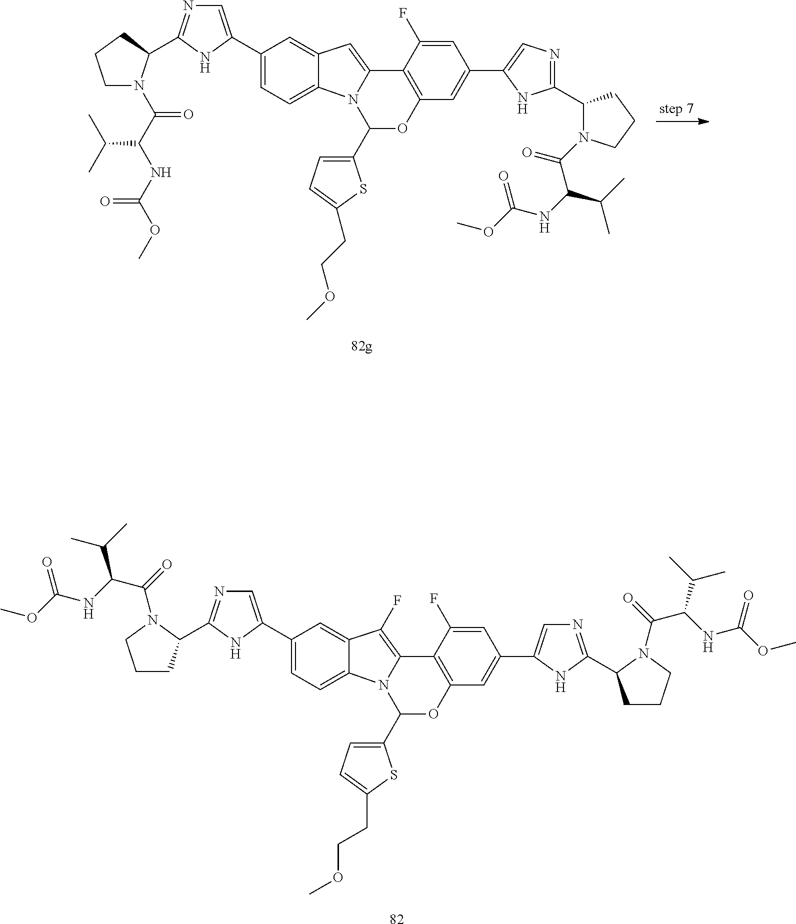

### Step 1

Core 2 (2.0 g, 10 mmol), compound 82a (2.0 g, 5.2 mmol), in anhydrous CH₃CN (20 mL) was added 5 drops of TFA at 25° C. The mixture was allowed to stir for 20 hours at 25° C. The reaction mixture became a clear solution and then a solid appeared. The product 82b was collected by filtration and washed with 20 mL of CH₃OH (2.62 g, 89.3% yield). LC/MS: Anal. Calcd. For [M+H]⁺ C23H18Br2FNO3S; 567.93. found 568.10.

### Step 2

A solution of 82b (5.65 g, 10 mmol) in dry toluene (50 mL) was added DDQ (3.4 g, 15 mmol). After refluxing for 2 hours, the solvent was removed in vacuo and diluted with EtOAc. The organic layer was washed with saturated Na₂SO₃ aqueous and brine, dried over Na₂SO₄, filtered and concentrated in vacuo. The residue obtained was washed with MeOH (300 mL), filtered and the solid just was the product 82c (4.50 g, 79.9% yield). LC/MS: Anal. Calcd. For [M+H]⁺ C23H16Br2FNO3S; 565.92. found 566.30.

### Step 3

A solution of 82c (2.0 g, 3.55 mmol) in THF (30 mL) was added LiOH (0.29 g, 7.1 mmol) at 25° C. The mixture was allowed to stir for 16 hours at 25° C. The solvent was removed in vacuo and diluted with EtOAc. The organic layer was washed with saturated Na₂SO₃ aqueous and brine, dried over Na₂SO₄, filtered and concentrated in vacuo. The solid just was the product 82d (1.76 g, 95.1% yield). LC/MS: Anal. Calcd. For [M+H]⁺ C21H14Br2FNO2S; 523.91. found 524.0.

### Step 4

A solution of 82d (1.2 g, 2.3 mmol) in DMF (10 mL) was added NaH (1.23 g, 30.7 mmol) at 0° C. The solution was allowed to stir for 20 minutes. Then the solution was added CH₃I (0.32 g, 2.3 mmol) from the dropping funnel. Water (50 mL) was added and solid appeared. The solvent was diluted with EtOAc (30 mL). The organic layer was washed with saturated Na₂SO₃ aqueous and brine, dried over Na₂SO₄, filtered and concentrated in vacuo. The residue obtained was purified using Flash column chromatography on silica gel (petroleum ether:ethyl acetate=100:1 to 100:6) to provide the product 82e (0.80 g, 65.0% yield). LC/MS: Anal. Calcd. For [M+H]⁺ C22H16Br2FNO2S; 537.92. found 538.10.

### Step 5

A suspension of 82e (0.8 g, 1.5 mmol), bis(pinacolato)diboron (1.2 g, 4.5 mmol), KOAc (0.9 g, 9 mmol) and Pd(dppf)Cl₂ (0.11 g, 0.15 mmol) in dioxane (25 mL) was allowed to stir at 115° C. under N₂ atmosphere 2 hours. The reaction mixture was cooled and concentrated; the residue obtained was washed with water (20 mL) and extracted with EtOAc (30 mL), washed with brine and dried over Na₂SO₄. After being concentrated in vacuo, the crude product was chromatographied on silica gel (petroleum ether:ethyl acetate=50:1 to 15:1) to provide compound 82f (0.70 g, 73.7% yield). LC/MS: Anal. Calcd. For [M+H]⁺ C34H40B2FNO6S; 632.27. found 632.0.

### Step 6

A suspension of compound 82f (1.13 g, 1.8 mmol), cap 31 (1.34 g, 3.6 mmol), Pd(dppf)Cl₂ (0.13 g, 0.18 mmol), Na₂CO₃ (1.14 g, 10.8 mmol) and in THF/H₂O (5:1, 54 mL) was refluxed at 75° C. for about 15 hours under N₂ atmosphere. After that, the mixture was concentrated in vacuo. The residue obtained was washed with water (20 mL) and extracted with EtOAc (400 mL), washed with brine and dried over anhydrous sodium sulfate. After being concentrated in vacuo, the residue obtained was purified using Flash column chromatography on silica gel (petroleum ether:ethyl acetate=4:1 to 1:100) to provide the product 82g (0.90 g, 52.1% yield). LC/MS: Anal. Calcd. For [M+H]⁺ C50H58FN9O8S; 964.41. found 964.3.

### Step 7

Compound 82 was got from compound 82g (340 mg) by SFC by using the following conditions:

Column: Chiralpak AS-H 250×4.6 mm

Solvent: 40% of iso-propanol (0.05% DEA) in CO₂

Flow rate: 2.5 mL/min

Wavelength: 220 nm

Compound 82 (160 mg, 47.06%). ¹H NMR (MeOD) δ: 7.76-7.91 (m, 1H), 7.57 (m, 1H), 7.18-7.42 (m, 5H), 6.88-7.06 (m, 2H), 6.52-6.53 (m, 1H), 6.40-6.44 (m, 1H), 5.07-5.15 (m, 2H), 4.18-4.20 (m, 2H), 3.97 (m, 2H), 3.84 (m, 2H), 3.61 (s, 6H), 3.38-3.43 (m, 2H), 3.19 (m, 3H), 2.83-2.86 (m, 2H), 1.99-2.30 (m, 12H), 0.86-0.93 (m, 12H). LC/MS: Anal. Calcd. For [M+H]⁺ C50H58FN9O8S; 964.41. found 964.3.

### Example 52

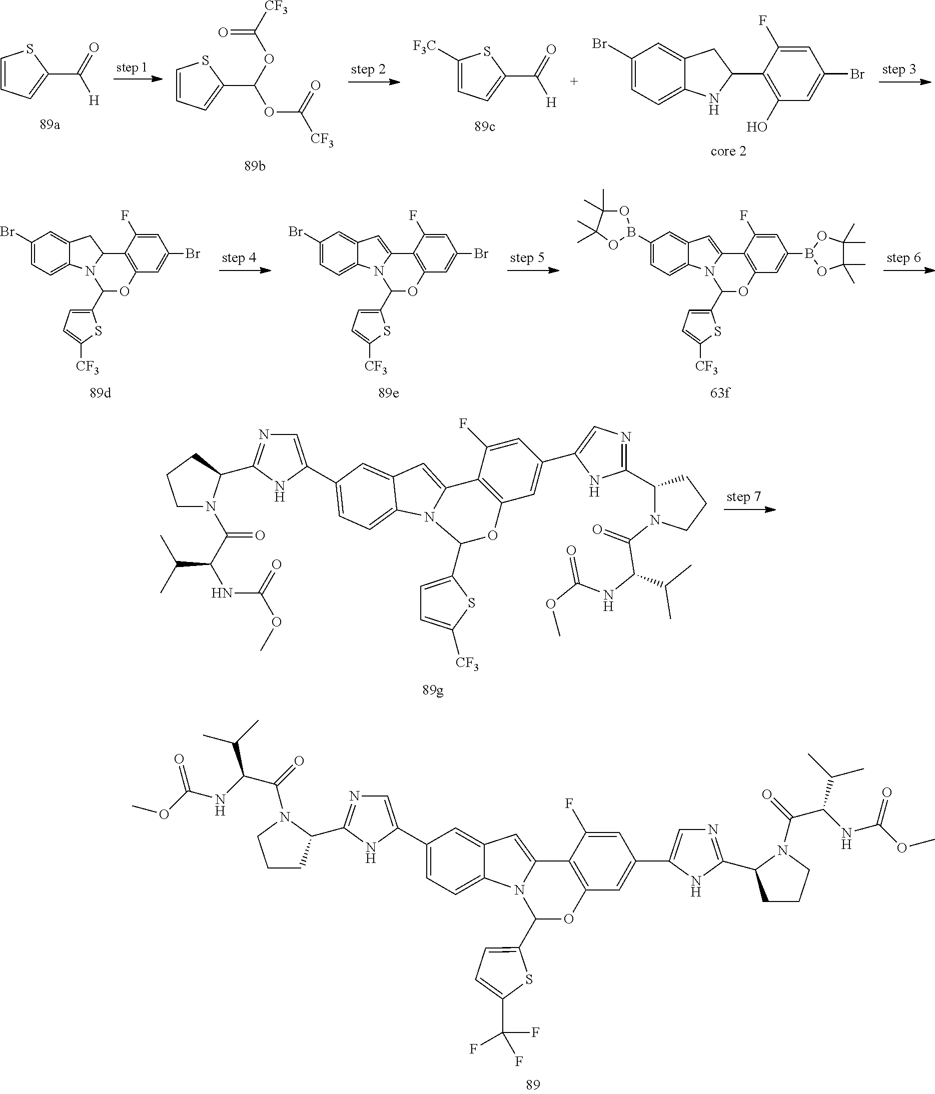

### Step 1

Compound 89a (5 g, 44.64 mmol) was added dropwise at 10° C. to a stirred mixture of TFAA (10.31 g, 49.11 mmol) and TFA (7 mg, 0.06 mmol). Then the reaction mixture was allowed to stir at 25° C. for 1 hour and sodium acetate (6 mg, 0.07 mmol) was added. The crude was distilled in vacuo (60-62° C.) to provide compound 89b (14.4 g, 81% yield). ¹H NMR (CDCl₃): δ 8.03 (s, 1H), 7.53-7.55 (m, 1H), 7.40-7.42 (m, 1H), 7.08-7.10 (m, 1H).

### Step 2

Xenon difluoride (2.624 g, 15.53 mmol) was added portionwise under N₂ at 0° C. to a stirred solution of compound 89b (5 g, 15.33 mmol) and TFA (1.77 g, 15.53 mmol) in DCM (50 mL) in a Teflon reaction bottle. Then the reaction mixture was allowed to stir at 25° C. for about 15 hours. The solution was poured into ice water and stirred for 1 hour. The organic phase was separated and the aqueous phase was extracted with DCM. The combined organic phases were washed with NaHCO₃ aqueous, brine, dried over Na₂SO₄ and concentrated in vacuo. The crude was purified using Pre-HPLC to provide compound 89c as yellow oil (190 mg, 7% yield). ¹H NMR (CDCl₃): δ 9.97 (s, 1H), 7.71-7.72 (m, 1H), 7.51-7.52 (m, 1H).

### Step 3

A mixture of compound 89c (100 mg, 0.26 mmol), core 2 (51 mg, 0.28 mmol) and trifluoroacetic acid (6 uL, 0.08 mmol) in MeCN (10 mL) was allowed to stir at 25° C. for about 15 hours. The solid was collected by filtration and washed with MeOH. The desired product compound 89d was obtained as a white solid (90 mg, 63% yield). ¹H NMR (CDCl₃): δ 7.25-7.30 (m, 3H), 7.07 (s, 1H), 6.79-6.86 (m, 4H), 5.07-5.09 (d, J=8.4 Hz, 1H), 3.57-3.63 (m, 1H), 3.27-3.31 (d, J=16.4 Hz, 1H).

### Step 4

To a solution of compound 89d (0.09 g, 0.16 mmol) in dry toluene (10 mL) was added DDQ (0.056 g, 0.25 mmol). After refluxing for 9 hours, the solvent was removed in vacuo and diluted with EtOAc. The organic layer was washed with saturated Na₂SO₃ aqueous and brine, dried over Na₂SO₄, filtered and concentrated in vacuo. The crude was purified using Pre-TLC to provide the desired product compound 89e as light red solid (88 mg, 99% yield). ¹H NMR (DMSO): δ 8.33 (s, 1H), 7.95 (s, 1H), 7.62-7.64 (d, J=8.4 Hz, 1H), 7.50-7.52 (m, 2H), 7.41-7.44 (m, 2H), 7.12-7.13 (d, J=3.2 Hz, 1H), 6.78-6.79 (d, J=4.0 Hz, 1H).

### Step 5

A suspension of the compound 89e (0.088 g, 0.16 mmol), bis(pinacolato)diboron (0.09 g, 0.35 mmol), KOAc (0.063 g, 0.64 mmol) and Pd(dppf)Cl₂ (0.012 g, 0.02 mmol) in dioxane (10 mL) was allowed to stir at 80° C. for 2 hours under N₂. The reaction mixture was cooled and concentrated in vacuo, and then the crude was purified using SiO₂ chromatography to provide the compound 89f as a white solid (99 mg, 97% yield). LC/MS: Anal. Calcd. For [M+H]⁺ C32H33B2FNO5S; 642.29. found 642.2.

### Step 6

A suspension of the compound 89f (0.5 g, 0.78 mmol), cap 31 (0.64 g, 1.72 mmol), Na₂CO₃ (0.413 g, 3.9 mmol) and Pd(dppf)Cl₂ (57 mg, 0.08 mmol) in

THF/H₂O (60 mL) was allowed to stir at 80° C. under N₂ atmosphere for about 15 hours. The reaction mixture was concentrated in vacuo and purified using Flash column chromatography on silica gel to provide the compound 89g (197 mg, 26% yield).

### Step 7

Compound 89 was separated from the compound 89g (197 mg) by SFC by using the following conditions:

Column: Chiralpak AS-H 250×4.6 mm

Solvent: 40% of iso-propanol (0.05% DEA) in CO₂

Flow rate: 2.5 mL/min

Wavelength: 220 nm

Compound 89 (60 mg, 30% yield). ¹H NMR (MeOH): δ 7.96-8.06 (m, 2H), 7.89 (s, 1H), 7.75 (s, 1H), 7.57-7.65 (m, 2H), 7.40 (d, J=10.56 Hz, 1H), 7.25-7.35 (m, 2H), 7.19 (s, 1H), 6.66 (d, J=3.13 Hz, 1H), 5.21 (dt, J=19.37, 7.34 Hz, 2H), 4.22 (t, J=7.04 Hz, 2H), 4.08 (br. s., 2H), 3.81-3.91 (m, 2H), 3.64 (s, 6H), 2.47-2.59 (m, 2H), 2.12-2.29 (m, 6H), 2.02-2.12 (m, 3H), 0.83-0.95 (m, 12H). LC/MS: Anal. Calcd. For [M+H]⁺ C48H51F4N9O7S; 974.03. found 974.0.

The following compounds of the present invention were made using the methods described in the Example above and substituting the appropriate reactants or reagents.

| | | | Observed |
|---|---|---|---|
| ID | Structure | Isomer | [M + H]+ |
| 89 90 | 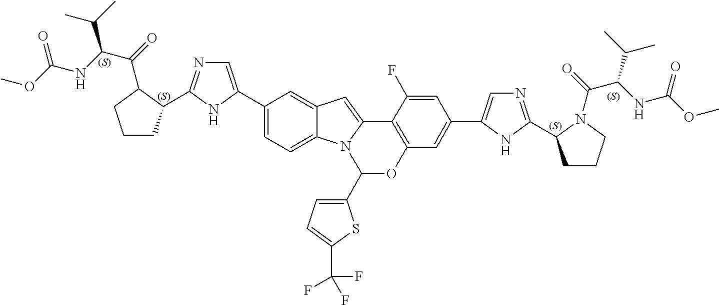 | Isomer 1 Isomer 2 | 974.4 974.4 |
| 722 | 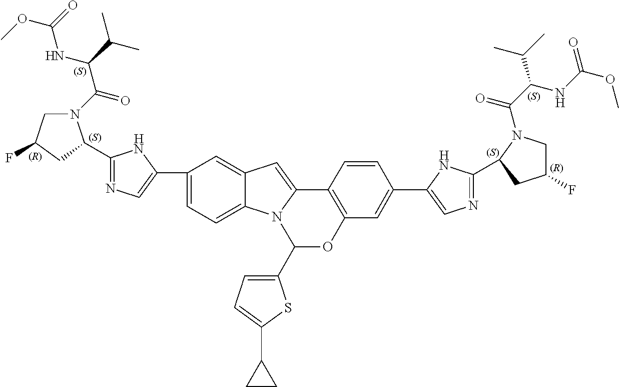 | Isomer 1 | 1006.4 |
| 734 | 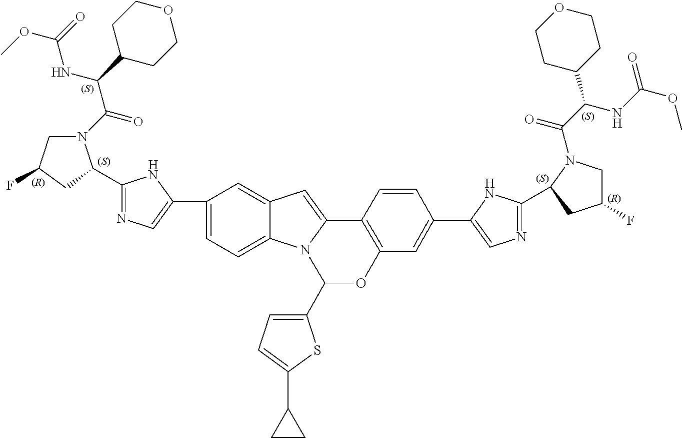 | racemic | 1048.0 |

### Example 53

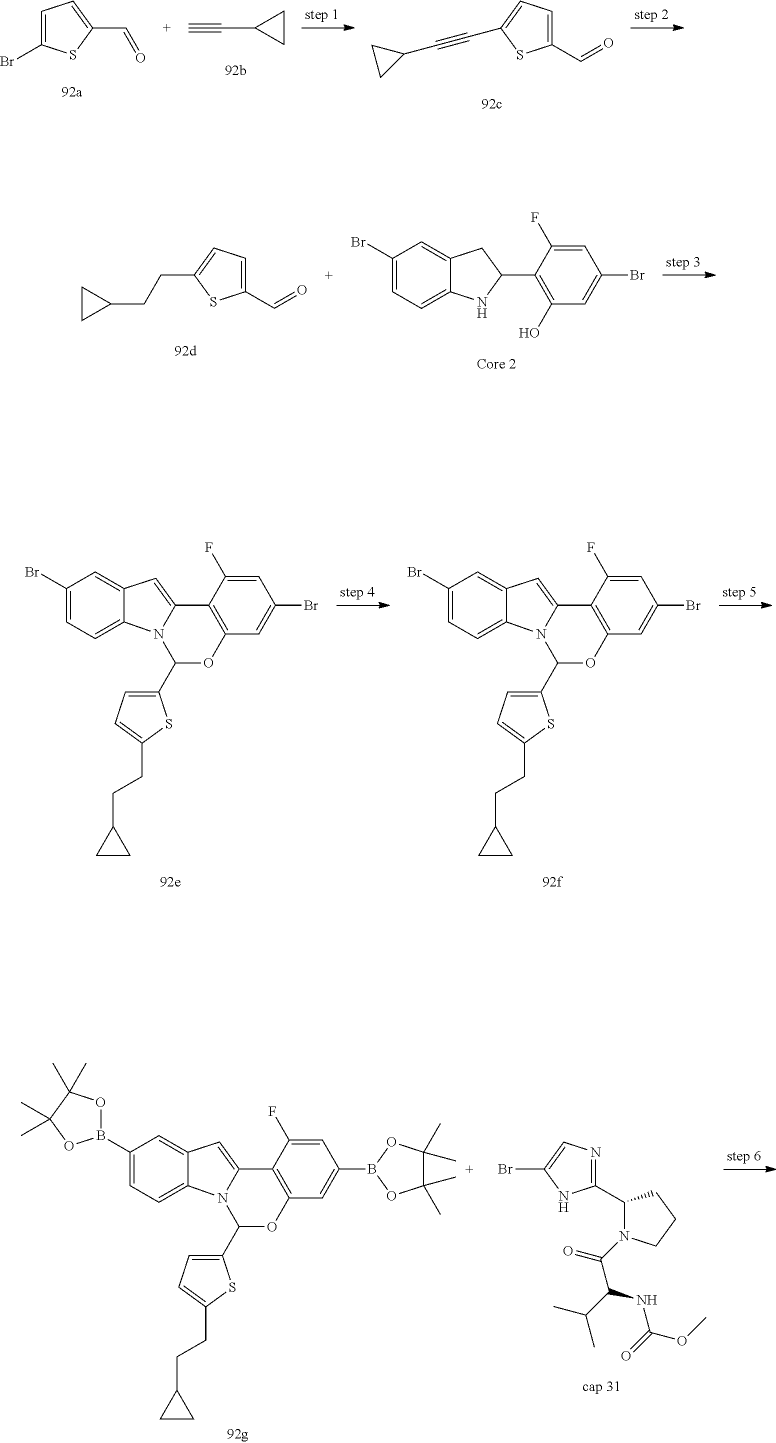

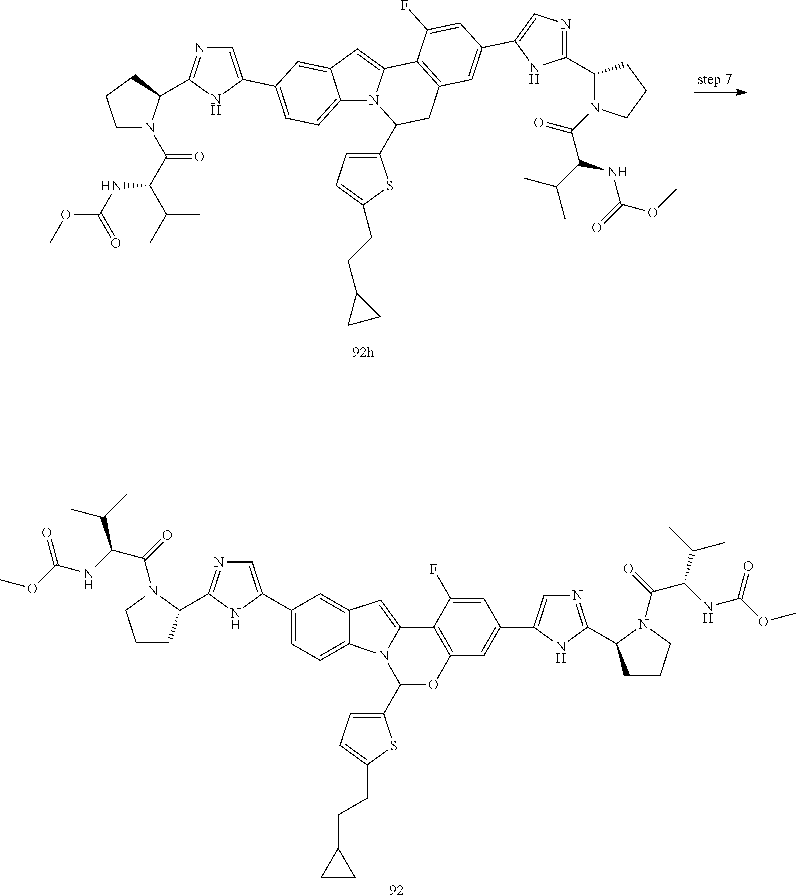

### Step 1

To a 100 mL flask was added 92a (7.6 g, 40 mmol) and 92b (3.3 g, 50 mmol), Pd(PPh₃)₂Cl₂ (0.87 g, 1.5 mmol), CuI (485 mg, 2.6 mmol) and THF/Et₃N (1:1, 30 mL). The solution was allowed to stir at ambient temperature 20 h under N₂ atmosphere. After that, the solution was concentrated under reduced pressure, extracted with EtOAc (100 mL), washed with brine and dried over anhydrous sodium sulfate and concentrated in vacuo. The crude product was purified using SiO₂ chromatographed (100 g, Petroleum Ether/EtOAc 90% to 95%) to provide 92c (5.0 g, 72%). LC/MS: Anal. Calcd. For [M+H]⁺ C10H8OS; 177.0. found 177.1.

### Step 2

To a solution of 92c (1.7 g, 10 mmol) in methanol (20 mL) was added 10% Pd/C (200 mg). The mixture was allowed to stir under H₂ (50 Psi) at 50 deg. for 3 hours, and filtered through celite. The filtrate was concentrated in vacuo to provide 92d (1.0 g, 56%). LC/MS: Anal. Calcd. For [M+H]⁺ C10H12OS; 180.1. found 181.2.

### Step 3

To a solution of 92d (1.35 g, 7.5 mmol), core 2 (1.9 g, 5 mmol) in anhydrous CH₃CN (30 mL) was added TFA (60 mg, 0.5 mmol). The mixture was allowed to stir 60 hours at ambient temperature. The reaction mixture became a clear solution and then a solid appeared. The solid was collected by filtration and washed with CH₃CN as 92e (0.9 g, 37.5%). LC/MS: Anal. Calcd. For [M+H]⁺ C24H20Br2FNOS; 548.0. found 500.0.

### Step 4

To a solution of 92e (950 mg, 1.73 mmol) in dry toluene (20 mL) was added DDQ (600 mg, 2.6 mmol). After refluxed for 2 hours, the solvent was removed in vacuo and diluted with EtOAc. The organic layer was washed with saturated Na₂SO₃ aqueous and brine, dried over Na₂SO₄, filtered and concentrated in vacuo. The residue obtained was washed with MeOH (10 mL), filtered and the solid just was 92f (0.7 g, 74%).

### Step 5

To a 50 mL flask was added 92f (0.75 g, 1.36 mmol), BIS(PINACOLATO)DIBORON (0.865 g, 3.4 mmol), KOAc (0.8 g, 8 mmol), Pd(dppf)Cl₂ (0.15 g, 0.2 mmol) and dioxane (15 mL). The mixture was allowed to stir at 100° C. under N₂ atmosphere for 2 hours. The reaction mixture was cooled and concentrated in vacuo. The crude product was purified using Flash column chromatography on silica gel (10 g, Petroleum Ether/EtOAc=5/95 to 20/80) to provide 92g (0.6 g, 69%). LC/MS: Anal. Calcd. For [M+H]⁺ C36H42B2FNO5S: 642.3. found 642.3.

### Step 6

To a 50 mL flask was added 92g (0.64 g, 1 mmol), cap 31 (1.1 g, 3 mmol), Na₂CO₃ (0.6 g, 6 mmol), Pd(dppf)₂Cl₂ (200 mg, 0.3 mmol) and THF/H₂O (5:1, 18 mL). The mixture was allowed to stir at 90° C. under N₂ atmosphere for 20 hours. After that, the mixture was extracted with EtOAc (30 mL), washed with brine, dried over anhydrous sodium sulfate, filtered. The solution was subjected directly to Pre-HPLC to provide 92h (0.44 g, 49%).

### Step 7

The two diastereo isomers (440 mg) were separated from 92h by SFC by using the following conditions.

Column: AS-H 4.6×250 mm

Solvent: 40% of iso-propanol (0.05% DEA) in CO2

Flow rate: 2.5 mL/min

Wavelength: 340 nm

Compound 92 (0.16 g, 36.3%). ¹H NMR (400 MHz, MeOH) δ: 7.94-7.97 (m, 1H), 7.71-7.84 (m, 3H), 7.12-7.35 (m, 4H), 6.46-6.53 (m, 2H), 5.14-5.23 (m, 2H), 4.19-4.21 (m, 2H), 3.84-4.06 (m, 4H), 3.62 (s, 6H), 2.70-2.74 (m, 2H), 2.47-2.53 (m, 2H), 2.02-2.24 (m, 8H), 1.34-1.39 (m, 2H), 0.86-0.91 (m, 12H), 0.55-0.61 (m, 1H), 0.29-0.31 (m, 2H), 0.06-0.08 (m, 2H). LC/MS: Anal. Calcd. For [M+H]⁺ C52H60FN9O7S; 974.4. found 974.2.

### Example 54

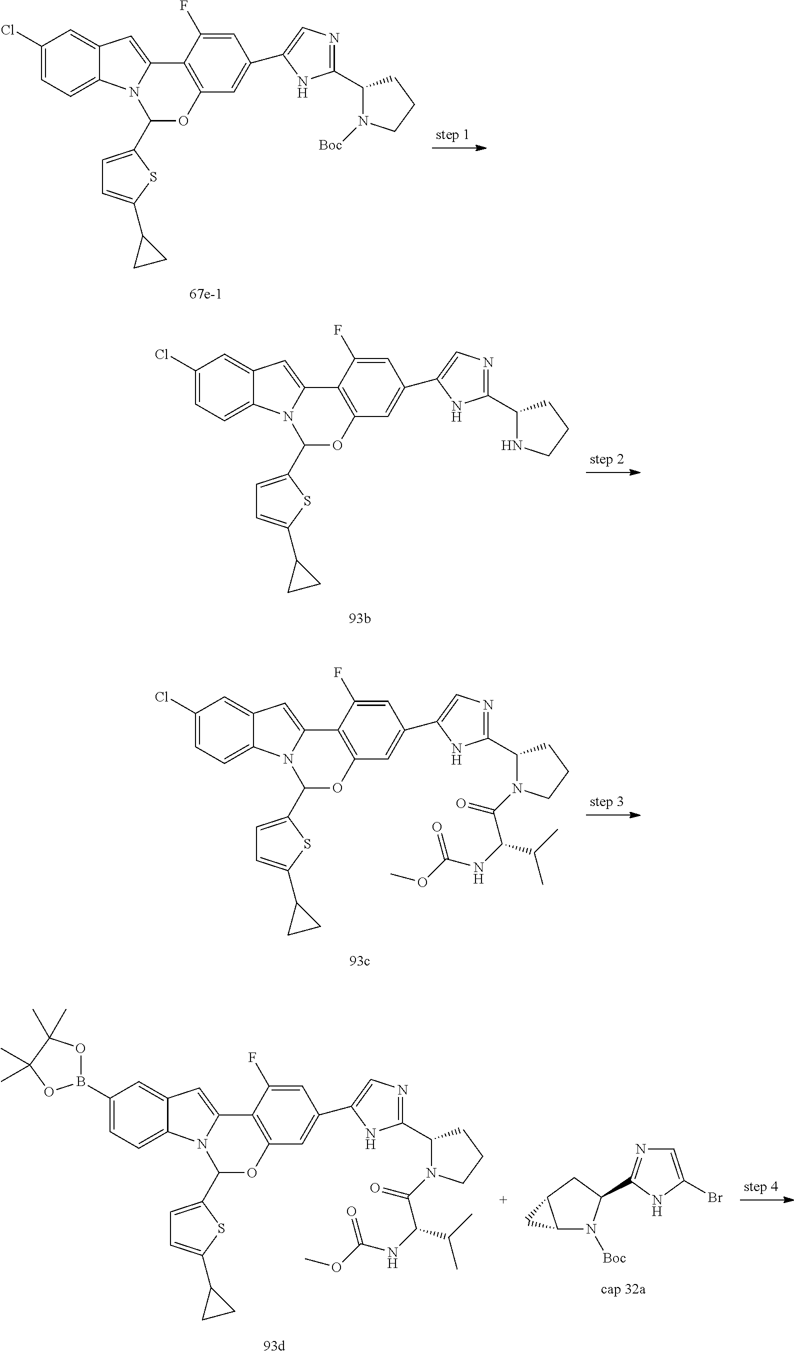 [SMILES: CC(C)(C)OC(=O)N1CCC[C@H]1c1ncc(-c2cc(F)c3c(c2)OC(c2ccc(C4CC4)s2)n2c-3cc3cc(Cl)ccc32)[nH]1 ; Fc1cc(-c2cnc([C@@H]3CCCN3)[nH]2)cc2c1-c1cc3cc(Cl)ccc3n1C(c1ccc(C3CC3)s1)O2 ; COC(=O)N[C@H](C(=O)N1CCC[C@H]1c1ncc(-c2cc(F)c3c(c2)OC(c2ccc(C4CC4)s2)n2c-3cc3cc(Cl)ccc32)[nH]1)C(C)C ; COC(=O)N[C@H](C(=O)N1CCC[C@H]1c1ncc(-c2cc(F)c3c(c2)OC(c2ccc(C4CC4)s2)n2c-3cc3cc(B4OC(C)(C)C(C)(C)O4)ccc32)[nH]1)C(C)C ; CC(C)(C)OC(=O)N1[C@@H]2C[C@@H]2C[C@H]1c1ncc(Br)[nH]1]

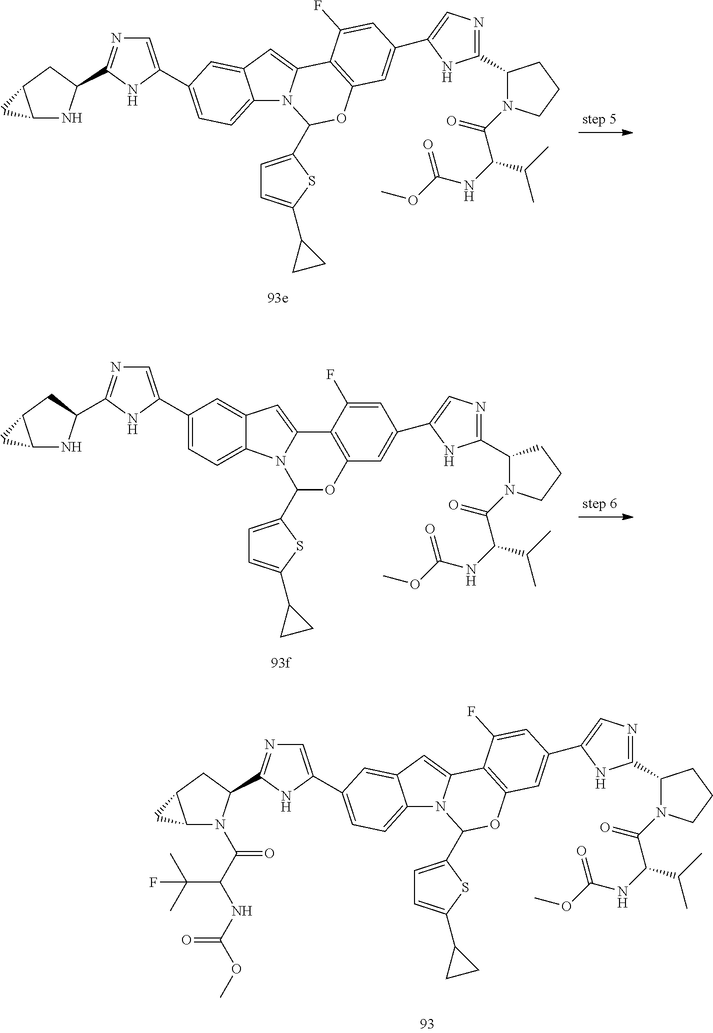 [SMILES: COC(=O)N[C@H](C(=O)N1CCC[C@H]1c1ncc(-c2cc(F)c3c(c2)OC(c2ccc(C4CC4)s2)n2c-3cc3cc(-c4cnc([C@@H]5C[C@H]6C[C@H]6N5)[nH]4)ccc32)[nH]1)C(C)C ; COC(=O)N[C@H](C(=O)N1CCC[C@H]1c1ncc(-c2cc(F)c3c(c2)OC(c2ccc(C4CC4)s2)n2c-3cc3cc(-c4cnc([C@@H]5C[C@H]6C[C@H]6N5)[nH]4)ccc32)[nH]1)C(C)C ; COC(=O)NC(C(=O)N1[C@@H]2C[C@@H]2C[C@H]1c1ncc(-c2ccc3c(c2)cc2n3C(c3ccc(C4CC4)s3)Oc3cc(-c4cnc([C@@H]5CCCN5C(=O)[C@@H](NC(=O)OC)C(C)C)[nH]4)cc(F)c3-2)[nH]1)C(C)(C)F]

### Step 1

Compound 67e-1 was made from Example 49. A suspension of 67e-1 (18 g, 28.5 mmol) in dioxane (100 mL) was added HCl/dioxane (4N, 100 mL) and stirred at 25° C. for 0.5 hour, then concentrated in vacuo to provide 93b (14 g, 96%). LC/MS: Anal. Calcd. For [M+H]⁺ C29H24ClFN4OS; 531.04. found 531.1.

### Step 2

To a mixture of 93b (14 g, 26.36 mol), cap 31c (4.61 g, 26.36 mmol) and HATU (10 g, 26.36 mmol) in DMF (100 mL) was DIPEA (7.0 g, 68.54 mmol). The resulting mixture was allowed to stir at 25° C. for 30 minutes, and LCMS judged the material was consumed up, and the mixture was poured into water (300 mL), extracted with EtOAc (200 mL×3), and the organic layer was washed with brine, dried over Na₂SO₄ and concentrated in vacuo. The crude product was purified using Flash column chromatography on silica gel (petroleum ether/ethyl acetate 10% to 80%) to provide 93c (16 g, 82%). LC/MS: Anal. Calcd. For [M+H]⁺ C36H35ClFN5O4S; 688.21. found 688.2.

### Step 3

To a mixture of 93c (11 g, 15.98 mmol), bis(pinacolato)diboron (4.0 g, 15.98 mmol), KOAc (3.13 g, 32 mmol), Pd₂(dba)₃ (560 mg, 0.8 mmol), X-Phos (380 mg 0.8 mmol) degassed and sealed under N₂ was added dry dioxane. Following further N₂ purging. The mixture was allowed to stir at 100° C. for about 15 hours. After that, cooling to 25° C., concentrated in vacuo and the residue obtained was purified using Flash column chromatography on silica gel o (petroleum ether/ethyl acetate 10% to 80%) to provide 93d (10.5 g, 87%). LC/MS Anal. Calcd. For [M+H]⁺ C42H47BFN5O6S; 779.73. found 780.

### Step 4

Compound cap 32a was prepared as described in Example 12A of 2012041014.

A mixture of 93d (1 g, 1.28 mmol), cap 32a (420 mg, 1.28 mmol), Na₂CO₃ (200 mg, 1.92 mmol) and Pd(dppf)Cl₂ (50 mg, 0.067 mmol) in THF/H₂O (v/v=10/1, 20 mL) was allowed to stir at 100° C. under N₂ atmosphere for about 15 hours. The reaction mixture was concentrated in vacuo and purified using Flash column chromatography on silica gel (10 g, petroleum ether/ethyl acetate 10% to 100%) to provide 93e (750 mg, 63%). LC/MS Anal. Calcd. For [M+H]⁺ C49H53FN8O6S; 901.4. found 901.1.

### Step 5

A suspension of 93e (700 mg, 0.77 mmol) in HCl/dioxane (4N, 2 mL) was allowed to stir at ambient temperature for 0.5 h, then concentrated in vacuo to provide compound 3f (600 mg, 97%). LC/MS Anal. Calcd. For [M+H]⁺ C44H45FN8O4S; 801.3. found 801.2.

### Step 6

To a mixture of 93f (600 mg, 0.75 mmol), Cap 1 (144 mg, 0.75 mmol) and HATU (290 mg, 0.75 mmol) in DMF (5 mL) was added DIPEA (340 mg, 2.63 mmol). The resulting mixture was allowed to stir at 25° C. for 30 minutes, and LCMS judged the material was consumed up, and the mixture was filtered and filtrate was purified using Pre-HPLC to provide 93 (200 mg, 26.6%). ¹H NMR (MeOD) δ: 7.97-7.82 (m, 1H), 7.63-7.65 (m, 2H), 7.45-7.47 (m, 2H), 7.36-7.38 (m, 1H), 7.26-7.28 (m, 1H), 7.15-7.04 (m, 1H), 6.98-7.01 (m, 1H), 6.51-6.36 (m, 2H), 5.21-5.08 (m, 2H), 5.01 (d, J=7.8 Hz, 1H), 4.22 (d, J=7.4 Hz, 1H), 4.02-4.08 (m, 1H), 3.88-3.90 (m, 2H), 3.65-3.66 (m, 6H), 2.72-2.57 (m, 1H), 2.53-2.42 (m, 1H), 2.35-2.36 (m, 1H), 2.29-2.19 (m, 1H), 2.18-1.96 (m, 4H), 1.89-1.91 (m, 1H), 1.56-1.40 (m, 3H), 1.37-1.22 (m, 3H), 1.08-1.10 (m, 1H), 1.02-0.81 (m, 9H), 0.50-0.52 (m, 2H). LC/MS Anal. Calcd. For [M+H]⁺ C51H55F2N9O7S; 976.10. found 976.9.

### Example 55

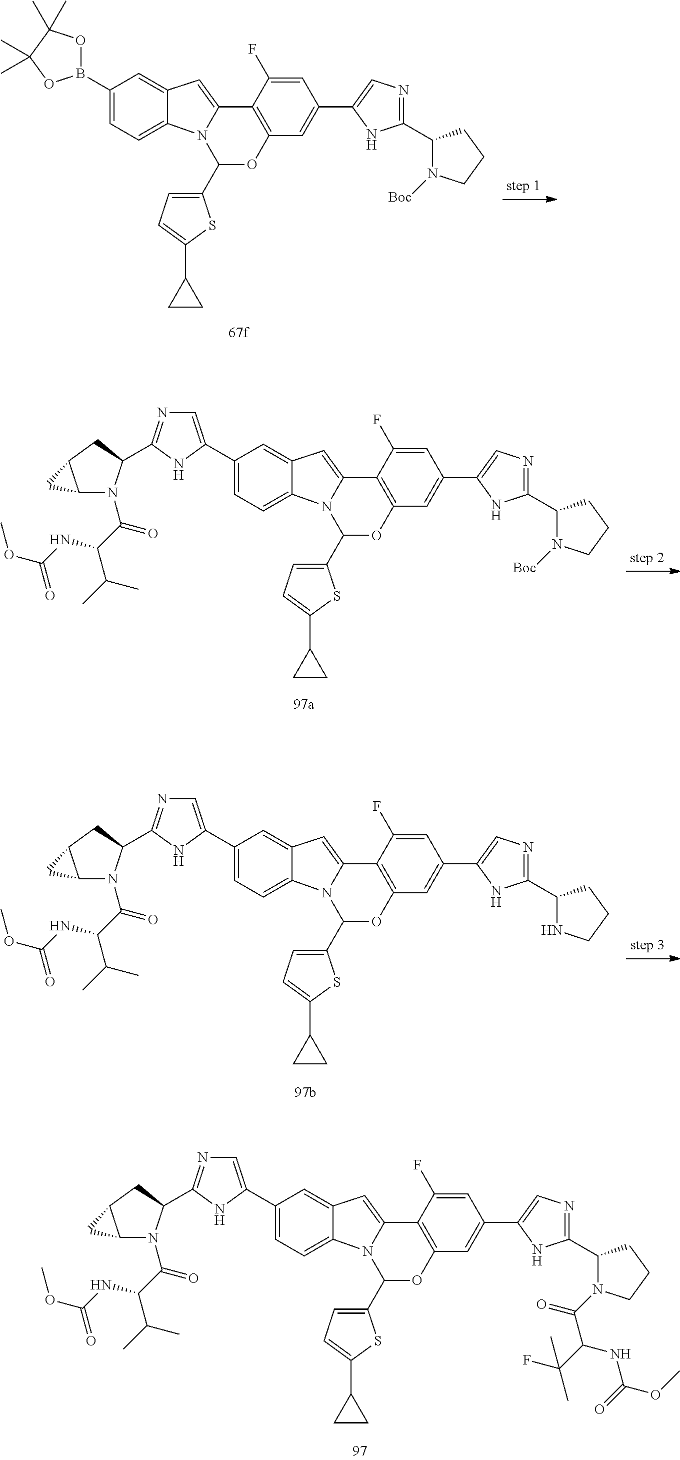

### Step 1

Compound 67f was made in Example 49. A suspension of compound 67f (0.4 g, 0.55 mmol), cap 32 (0.23 g, 0.61 mmol), Na₂CO₃ (0.17 g, 1.65 mmol) and Pd(dppf)Cl₂ (40 mg, 0.055 mmol) in THF/H₂O (10 mL) was allowed to stir at 90° C. under N₂ atmosphere for about 15 hours. The reaction mixture was concentrated in vacuo and purified using Flash column chromatography on silica gel (10 g, 1% to 2%) to provide 97a (0.2 g, 40.8%). LC/MS: Anal. Calcd. For [M+H]⁺ C49H53FN8O6S: 901.4. found 901.6.

### Step 2

To a solution of 97a (0.2 g, 0.2 mmol) in dioxane (5 mL) was added HCl/dioxane (5 mL, 4M) and stirred at ambient temperature for 2 hours, then concentrated in vacuo and dried under high vacuum to provide 97b (0.16 g, 100%). LC/MS: Anal. Calcd. For [M+H]⁺ C44H45FN8O4S; 801.3. found 801.4.

### Step 3

To a mixture of 97b (88 mg, 0.11 mmol), cap 1 (24 mg, 0.12 mmol) and DIPEA (0.5 mL) in DMF (3 mL) was added HATU (46 mg, 0.12 mmol). The resulting mixture was allowed to stir at 25° C. for 2 hours. The solution was directly purified by Pre-HPLC to provide the desired compound 97 (60 mg, 56.1%). ¹H NMR (MeOD) δ: 8.01 (s, 1H), 7.83 (s, 1H), 7.80 (br. s., 1H), 7.74 (s, 1H), 7.45-7.55 (m, 2H), 7.37 (d, J=10.56 Hz, 1H), 7.31 (br. s., 1H), 7.19 (br. s., 1H), 6.50 (br. s., 1H), 6.46 (d, J=3.52 Hz, 1H), 5.21 (t, J=7.04 Hz, 1H), 5.07-5.15 (t, J=7.04 Hz, 1H), 4.71 (d, J=9.39 Hz, 1H), 4.54 (d, J=6.26 Hz, 1H), 4.01 (d, J=4.70 Hz, 1H), 3.88-3.96 (m, 1H), 3.80 (br. s., 1H), 3.67 (d, J=6.65 Hz, 6H), 2.62-2.73 (m, 1H), 2.47 (br. s., 2H), 2.01-2.31 (m, 5H), 1.89-1.99 (m, 1H), 1.47 (s, 1H), 1.41 (s, 1H), 1.30 (s, 1H), 1.24 (s, 1H), 1.09 (d, J=8.22 Hz, 1H), 1.00 (d, J=6.65 Hz, 3H), 0.92 (d, J=6.26 Hz, 6H), 0.50-0.59 (m, 2H). LC/MS: Anal. Calcd. For [M+H]⁺ C51H55F2N9O7S; 976.4. found 976.5.

### Example 56

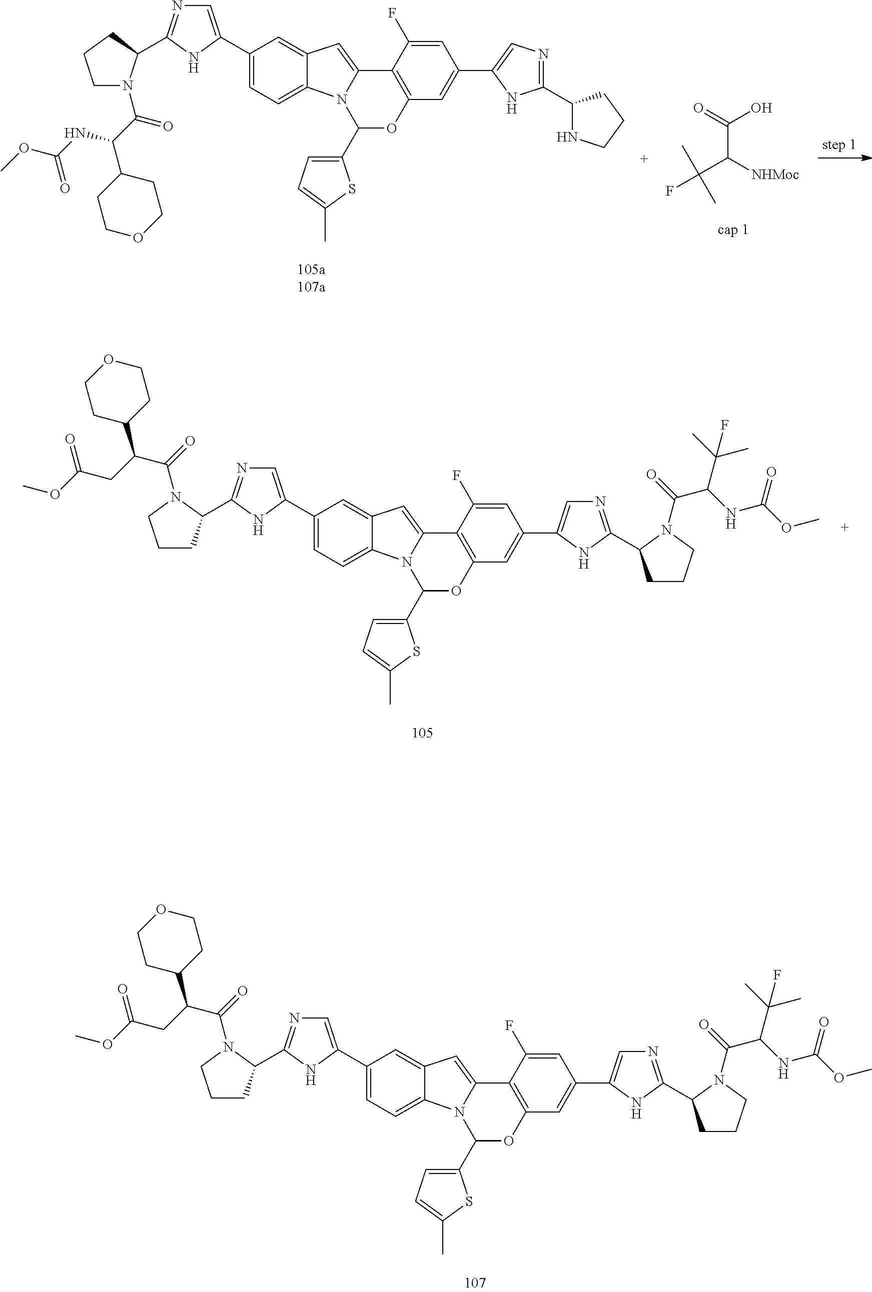

### Step 1

Compound 105a and 107a were made from Example 13. A suspension of 105a (150 mg, 0.165 mmol), cap1 (32.0 mg, 0.166 mmol) and DIPEA (9 drops) in DMF (3 mL) was added HATU (76 mg, 0.19 mmol). The resulting mixture was allowed to stir at 25° C. for 2 hours. LCMS judged the material was consumed up. The crude product purified using Pre-HPLC to provide 105 (67.0 mg, 41.4%). ¹H NMR (MeOD) δ: 7.75-8.01 (m, 4H), 7.32-7.54 (m, 4H), 7.20 (s, 1H), 6.48-6.52 (m, 2H), 5.22-5.24 (m, 2H), 4.70-4.72 (m, 1H), 3.83-4.29 (m, 7H), 3.64-3.66 (m, 6H), 2.56-2.58 (m, 2H), 2.14-2.32 (m, 11H), 1.24-1.62 (m, 10H). LC/MS: Anal. Calcd. For [M+H]⁺ C50H55F2N9O8S; 980.4. found 980.4.

Compound 107 was prepared following the same procedure with 105 (60 mg, 31.7%). ¹H NMR (MeOD) δ: 7.74-8.00 (m, 4H), 7.30-7.50 (m, 4H), 7.18 (s, 1H), 6.46-6.51 (m, 2H), 5.15-5.24 (m, 2H), 4.68-4.71 (m, 1H), 3.87-4.27 (m, 7H), 3.64-3.66 (m, 6H), 2.53-2.56 (m, 2H), 2.13-2.31 (m, 11H), 1.22-1.45 (m, 10H). LC/MS: Anal. Calcd. For [M+H]⁺ C50H55F2N9O8S; 980.4. found 980.4.

### Example 57

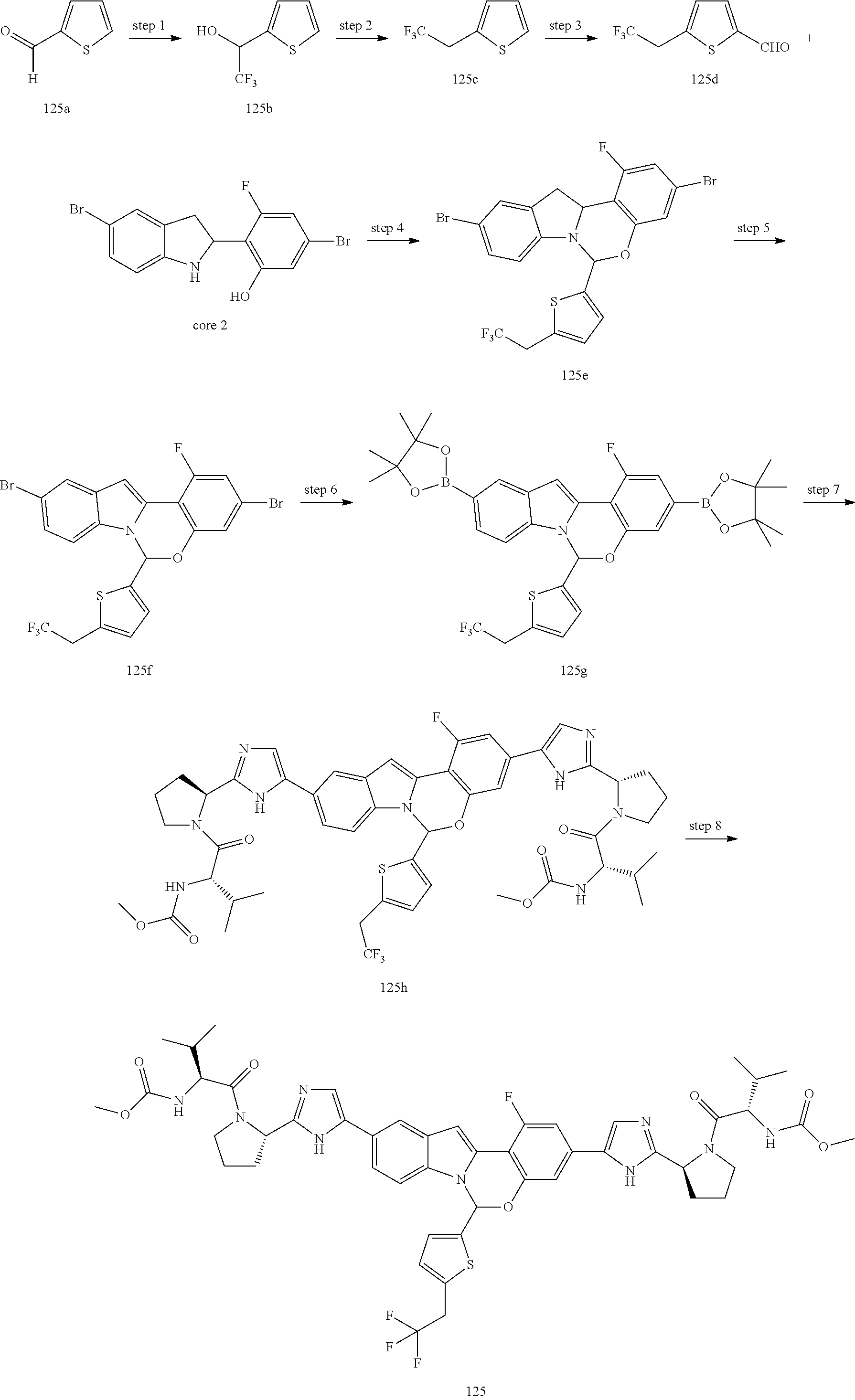

### Step 1

To a suspension of 125a (11.2 g, 0.1 mol) and CsF (1.52 g, 0.01 mol) in DME (70 mL) was added TMSCF₃ (28.4 g, 0.2 mol) dropwise at 0° C. Then the mixture was allowed to stir at 25° C. for 3 hours. The starting material was consumed up. After that, HCl (3N) was added to quench the reaction and it was allowed to stir for another 0.5 hours. The intermediate was used up and the desired product was formed. Extracted with ethyl acetate, dried over anhydrous sodium sulfate, concentrated in vacuo and purified using Flash column chromatography on silica gel (200 g, Petroleum Ether/EtOAc=1% to 2%) to provide 125b as a oil (17.2 g, 97% yield). ¹H NMR (CDCl₃): 7.39-7.38 (m, 1H), 7.19-7.18 (d, 1H, J=3.6 Hz), 7.05-7.03 (m, 1H), 5.29-5.23 (m, 1H).

### Step 2

To a solution of 125b (6 g, 33.71 mmol) in HOAc (50 mL) was added concentrated HCl (25 mL) and SnCl₂.2H₂O (38 g, 168.5 mmol). Then the mixture was allowed to stir at 80° C. for about 15 hours. Checked by LCMS, the starting material was consumed up. Extracted with CH₂Cl₂, washed with water, saturated sodium hydrogen carbonate and brine, concentrated in vacuo at ambient temperature to provide 125c as oil which was used in next step directly (4 g, 69% yield). ¹H NMR (CDCl₃): 7.30-7.26 (m, 1H), 7.01-7.00 (m, 2H), 3.68-3.58 (m, 2H).

### Step 3

To a solution of 125c (3.8 g, 22.89 mmol) in MSA (30 mL) was added urotropine (3.85 g, 27.47 mmol) slowly. After that, the reaction was allowed to stir at 75° C. for 3 hours. Detected by TLC, the starting material was consumed up. Extracted with CH₂Cl₂, washed with water, saturated sodium hydrogen carbonate to pH=8 and brine, concentrated in vacuo and purified using Flash column chromatography on silica gel to provide 125d as oil (200 mg, 5% yield). ¹H NMR (CDCl₃): 9.89 (s, 1H), 7.69-7.68 (m, 1H), 7.15-7.14 (m, 1H), 3.69-3.62 (m, 2H).

### Step 4

To a mixture of 125d (350 mg, 1.8 mmol) and core 2 (636 mg, 1.64 mmol) in anhydrous CH₃CN (8 mL) was added TFA (56 mg, 0.49 mmol) at 25° C. The mixture was agitated for 6 hours at 25° C. The reaction mixture became a clear solution and then a solid appeared. The solid was collected by filtration and washed with CH₃CN to provide 125e (0.85 g, 92% yield). LC/MS: Anal. Calcd. For [M+H]⁺ C₂₁H₁₄Br₂F₄NOS; 563.9. found 563.2.

### Step 5

The solution of 125e (850 mg, 1.51 mmol) in dry toluene (20 mL) was added DDQ (514 mg, 2.26 mmol). After refluxed for 2 hours, the solvent was removed in vacuo and diluted with EtOAc. The organic layer was washed with saturated Na₂SO₃ aqueous and brine, dried over Na₂SO₄, filtered and concentrated in vacuo. The residue obtained was washed with MeOH (10 mL), filtered and the solid just was the product to provide 125f (0.7 g, 82% yield). LC/MS: Anal. Calcd. For [M+H]⁺ C₂₁H₁₂Br₂F₄NOS; 561.9. found 561.2.

### Step 6

To a solution of 125f (600 mg, 1.07 mmol) in dioxane was added BIS(PINACOLATO)DIBORON (650 mg, 2.56 mmol) and Pd(dppf)Cl₂ (78 mg, 0.11 mmol) and KOAc (419 mg, 4.28 mmol). The reaction mixture was allowed to stir under N₂ and heated to 110° C. for about 15 hours. After that, the solvent was removed in vacuo, and the residue obtained was purified using Flash column chromatography on silica gel (10 g, Petroleum Ether/EtOAc=10/1) to provide 125g (0.6 g, 86%). LC/MS: Anal. Calcd. For [M+H]⁺ C₃₃H₃₆B₂F₄NO₅S; 656.24. found 656.18.

### Step 7

A suspension of 125g (400 mg, 0.61 mmol), cap 31 (455 mg, 1.22 mmol), Pd(dppf)Cl₂ (45 mg, 0.06 mmol), Na₂CO₃ (259 mg, 2.44 mmol) and in THF/H₂O (10:1, 33 mL) was refluxed at 75° C. for about 15 hours under N₂ atmosphere. After that, the mixture was filtered; the filtrate was washed with water (50 mL) and extracted with EtOAc (100 mL), washed with brine, dried over anhydrous sodium sulfate and concentrated in vacuo. The residue obtained was purified using Pre-HPLC to provide 125h (200 mg, 33% yield).

### Step 8

The two diastereo isomers of 125h (200 mg) were separated by SFC by using the following conditions to provide 125.

Column: Chiralpak AS-H 250×4.6 mm I.D., 5 um

Solvent: 40% of iso-propanol (0.05% DEA) in CO₂

Flow rate: 2.5 mL/min

Wavelength: 340 nm

Isomer: 125 (60 mg, 60%). ¹H NMR (MeOD). δ: 8.02 (s, 1H), 7.90-7.90 (m, 2H), 7.76 (m, 1H), 7.53 (m, 1H), 7.43-7.34 (m, 2H), 7.21 (br. s., 1H), 6.82-6.81 (d, 1H), 6.62-6.61 (d, 1H), 5.25-5.17 (m, 2H), 4.23-4.19 (m, 2H), 4.09 (m, 2H), 3.86 (m, 2H), 3.66 (s, 6H), 3.61-3.56 (m, 2H), 2.57-2.51 (m, 2H), 2.27-2.03 (m, 8H), 0.93-0.87 (m, 12H). LC/MS: Anal. Calcd. For [M+H]⁺ C₄₉H₅₃F₄N₉O₇S; 988.4. found 988.3.

### Example 58

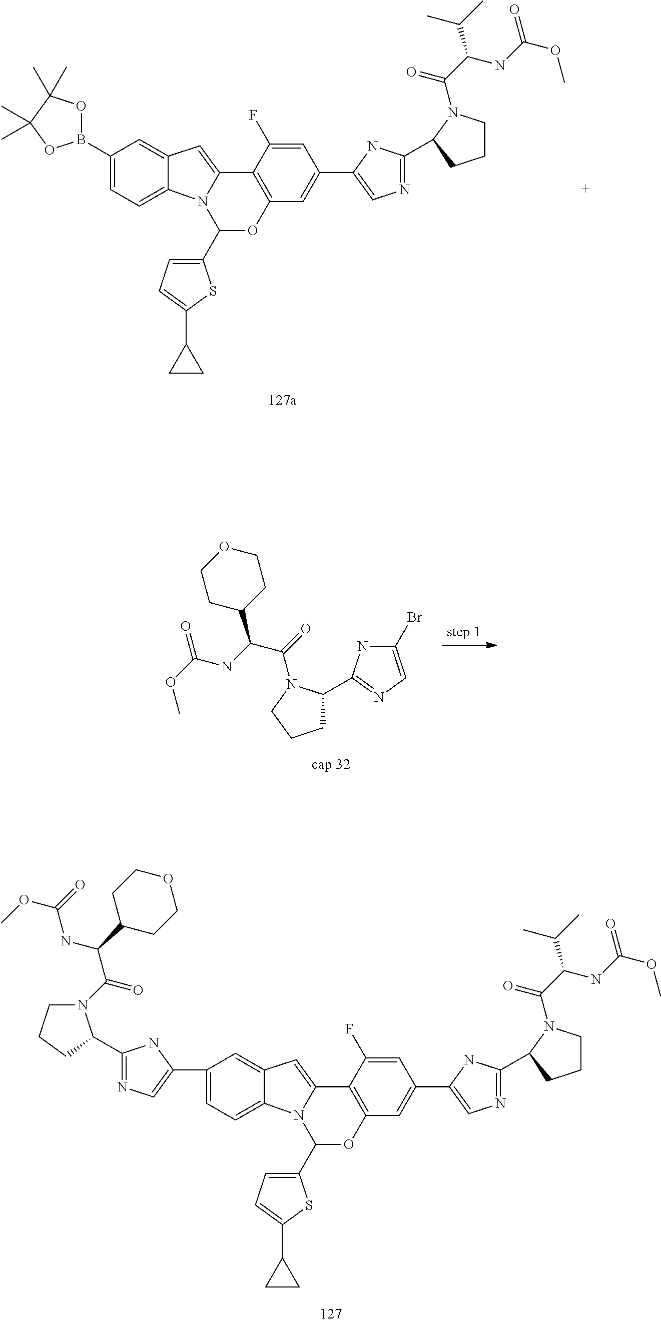

Compound 127a was in a similar method as compound 2h in Example 33. A mixture of 127a (10.5 g, 13.46 mmol), cap 32 (5.6 g, 13.46 mmol), Na₂CO₃ (3.56 g, 33.65 mmol) and Pd(dppf)Cl₂ (450 mg, 0.67 mmol) in THF/H₂O (v/v=10/1, 300 mL) was allowed to stir at 100° C. under N₂ for about 15 hours. The reaction mixture was concentrated in vacuo and purified using chromatography on silica gel (Petroleum Ether/EtOAc 10% to 100%) to provide the 127 (4.9 g, 36.8% yield). ¹H NMR (MeOD) δ: 8.00 (s, 1H), 7.90 (s, 1H), 7.76-7.80 (m, 2H), 7.57-7.44 (m, 2H), 7.38-7.40 (m, 1H), 7.29 (s, 1H), 7.15-7.16 (m, 1H), 6.55-6.42 (m, 2H), 5.29-5.14 (m, 2H), 4.27 (d, J=8.2 Hz, 1H), 4.20 (d, J=7.0 Hz, 1H), 4.09-4.11 (m, 2H), 3.99-3.79 (m, 4H), 3.64 (s, 6H), 3.32-3.41 (m, 4H), 2.61-2.46 (m, 2H), 2.32-2.09 (m, 6H), 2.07-1.88 (m, 3H), 1.67-1.29 (m, 4H), 0.89-0.92 (m, 8H), 0.51-0.53 (m, 2H). LC/MS: Anal. Calcd. For [M+H]⁺ C52H58FN9O8S; 988.14. found 988.5.

### Example 59

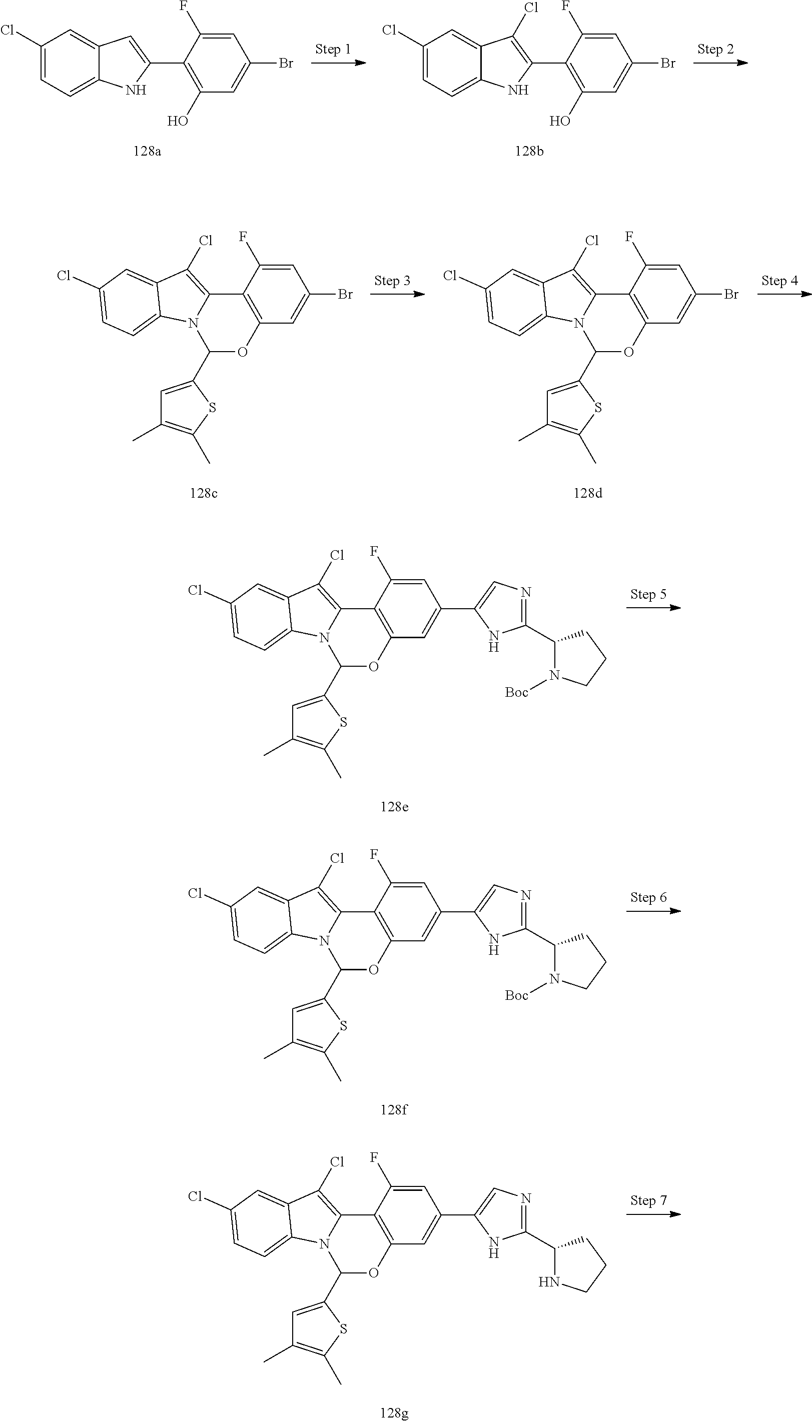

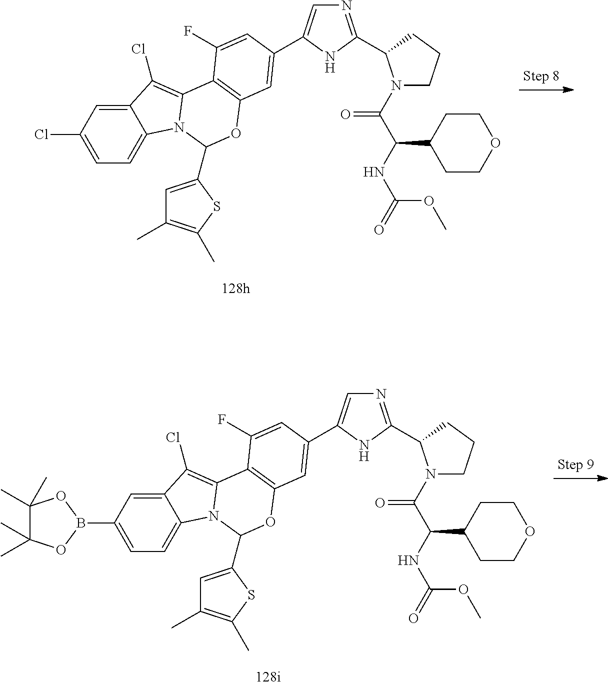 [SMILES: COC(=O)N[C@@H](C(=O)N1CCC[C@H]1c1ncc(-c2cc(F)c3c(c2)OC(c2cc(C)c(C)s2)n2c-3c(Cl)c3cc(Cl)ccc32)[nH]1)C1CCOCC1 ; COC(=O)N[C@@H](C(=O)N1CCC[C@H]1c1ncc(-c2cc(F)c3c(c2)OC(c2cc(C)c(C)s2)n2c-3c(Cl)c3cc(B4OC(C)(C)C(C)(C)O4)ccc32)[nH]1)C1CCOCC1]

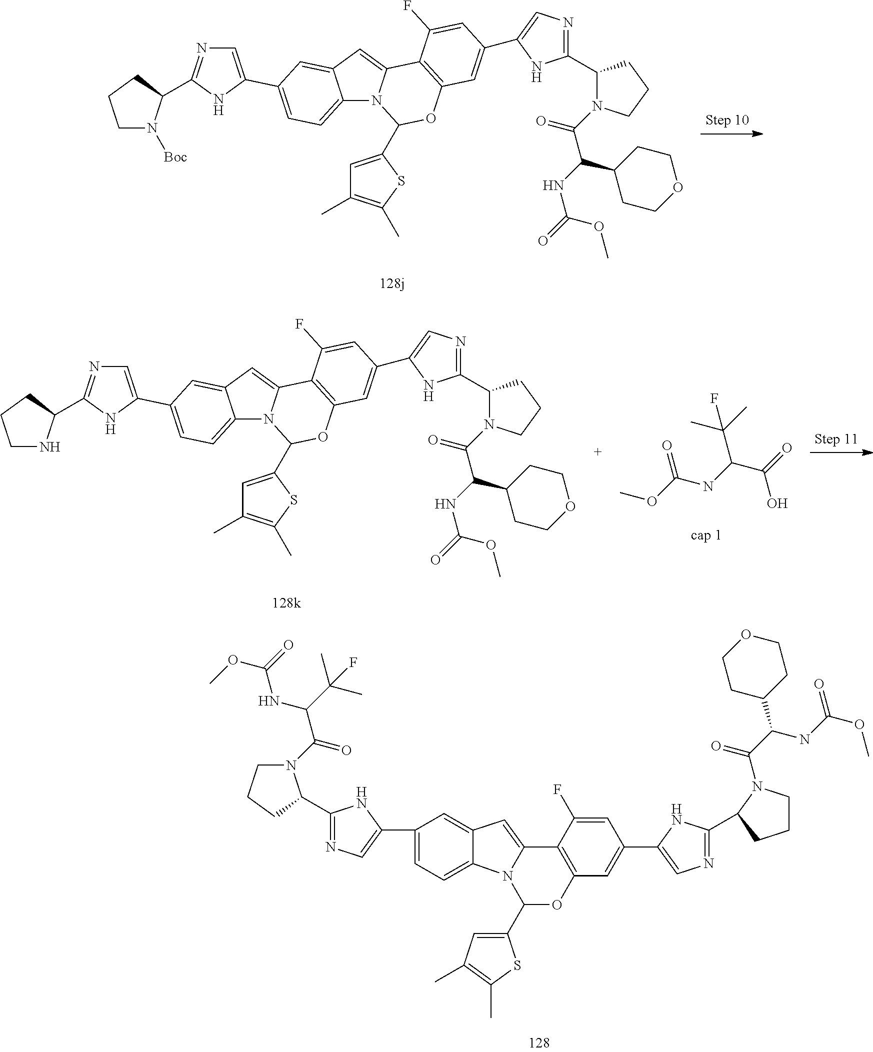

### Step 1

Compound 128a was prepared from Example 19 of International Publication No. 2012/040923 A1. To a solution of 128a (11 g, 0.033 mol) in anhydrous DMF (100 mL) was added NCS (4.5 g, 0.034 mol) at 25° C. The mixture was allowed to stir at 25° C. for about 15 hours, before poured into water and extracted with ethyl acetate. The organic layer was dried over Na₂SO₄. After filtration and concentration, the residue obtained was purified using Flash column chromatography on silica gel (100 g, Hexane/EtOAc 0% to 20%) to provide 128b (11.5 g, 95% yield).

### Step 2

To a mixture of 4,5-dimethylthiophene-2-carbaldehyde (140 mg, 1 mmol) and 128b (0.14 g, 0.38 mmol) in anhydrous toluene (10 mL) was added MSA (0.1 mmol). The mixture was heated at 120° C. for about 15 hours. After cooling to 20° C., the residue obtained was purified using Flash column chromatography on silica gel (5 g, Hexane/EtOAc 0% to 2%) to provide 128c (110 mg, 59.5% yield).

### Step 3

To a solution of 128c (110 mg, 0.223 mmol) in dioxane was added BIS(PINACOLATO) DIBORON (124 mg, 0.49 mmol) and Pd(dppf)Cl₂ (33 mg, 0.045 mmol) and KOAc (110 mg, 1.11 mmol). The reaction mixture was allowed to stir under N₂ and heated to 110° C. for 4 hours. After cooling to room temperature and concentration, the residue obtained was purified using Flash column chromatography on silica gel (5 g, Hexane/EtOAc 0% to 10%) to provide 128d (110 mg, 83% yield).

### Step 4

A suspension of 128d (1.0 g, 1.84 mmol), cap 31a, Pd(dppf)Cl₂ (130 mg, 0.18 mmol), Na₂CO₃ (1 g, 9.2 mmol) in THF/H₂O/DMF (5:2:1, 32 mL) was refluxed for about 15 hours under N₂ atmosphere. Then the reaction mixture was poured into water, and extracted with ethyl acetate. The organic layer was dried over Na₂SO₄. After filtration and concentration, the residue obtained was purified using Flash column chromatography on silica gel (10 g, Hexane/EtOAc 0% to 10%) to provide 128e (1 g, 83.3% yield). LC/MS: Anal. Calcd. For [M+H]⁺ C33H31C12FN4O3S; 653.2. found 653.2.

### Step 5

The compound of 128e (1 g) was separated by SFC by using the following conditions to provide 128f (0.42 g, 84%).

Column: AD 250 mm*50 mm, 10 um

Solvent: Supercritical CO₂, B: EtOH(0.05% NH3H2O), A:B=50:50

Flow rate: 220 mL/min

Wavelength: 220 nm

### Step 6

The compound of 128f (416 mg, 0.638 mmol) in 5 mL of dioxane was added into HCl/dioxane (10 mL). Then the mixture was allowed to stir at 20° C. for 2 hours. When the reaction completed, the mixture was concentrated in vacuo to provide 128g (0.35 g, 98%).

### Step 7

To a mixture of 128g (350 mg, 0.64 mmol), cap 32c (140 mg, 0.64 mmol) and DIPEA (0.6 mL) in DMF (10 mL) was added HATU (266 mg, 0.7 mmol). The resulting mixture was allowed to stir at 20° C. for about 15 hours. The mixture was poured into water and extracted with ethyl acetate. The organic layer was washed with brine and dried over Na₂SO₄. After filtration and concentration, the residue obtained was purified using Flash column chromatography on silica gel (4 g, DCM/MeOH 0% to 10%) to provide 128h (0.4 g, 83%). LC/MS: Anal. Calcd. For [M+H]⁺ C37H36C12FN5O5S; 752.18. found 752.2.

### Step 8

To a mixture of 128h (510 mg, 0.68 mmol), BIS(PINACOLATO)DIBORON (345 mg, 1.36 mmol), KOAc (266 mg, 2.72 mmol), Pd₂(dba)₃ (124 mg, 0.136 mmol), X-Phos (130 mg, 0.272 mmol) degassed and sealed under N₂ was added dry dioxane. The mixture was allowed to stir at 100° C. for about 15 hours. After cooling to 20° C., the residue obtained was purified using Flash column chromatography on silica gel (10 g, DCM/MeOH 0% to 5%) to provide 128i (0.5 g, 90%).

### Step 9

A suspension of 128i (550 mg, 0.68 mmol), cap 31a (237 mg, 0.75 mmol), Pd(dppf)Cl₂ (50 mg, 0.068 mmol), Na₂CO₃ (216 mg, 2.04 mmol) and THF/H₂O/DMF (5:2:1, 32 mL) was refluxed for about 15 hours under N₂ atmosphere. Then the reaction mixture was poured into water, and extracted with ethyl acetate. The organic layer was dried over Na₂SO₄. After filtration and concentration, the residue obtained was purified using Flash column chromatography on silica gel (4 g, DCM/MeOH 0% to 5%) to provide 128j (0.5 g, 80%). LC/MS: Anal. Calcd. For [M+H]⁺ C49H55FN8O7S; 918.4. found 918.6.

### Step 10

The compound of 128j (494 mg, 0.538 mmol) in dioxane (5 mL) was added into HCl/dioxane (5 mL). Then the mixture was allowed to stir at 20° C. for 3 hours. When the reaction completed, the mixture was concentrated in vacuo to provide 128k (0.4 g, 91%).

### Step 11

To a mixture of 128k (150 mg, 0.18 mmol), cap 1 (36 mg, 0.183 mmol) and DIPEA (0.2 mL) in DMF (5 mL) was added HATU (70 mg, 0.183 mmol). The resulting mixture was allowed to stir at 20° C. for about 15 hours, and then was subjected directly to Pre-HPLC to provide 128 (50 mg, 27.5% yield). ¹H NMR (MeOD) δ: 8.01 (s, 1H), 7.94 (s, 1H), 7.79 (s, 1H), 7.74 (s, 1H), 7.54-7.52 (m, 2H), 7.38-7.35 (m, 2H), 7.19 (s, 1H), 6.35 (s, 1H), 5.25-5.15 (m, 2H), 4.72-4.70 (d, J=8.8 Hz, 1H), 4.24-4.22 (d, J=8.4 Hz, 1H), 4.11-4.00 (m, 3H), 3.93-3.84 (m, 4H), 3.65 (s, 6H), 3.36-3.32 (m, 2H), 2.57-2.53 (m, 2H), 2.26-2.15 (m, 10H), 1.19 (s, 3H), 1.46-1.40 (s, 6H), 1.28-1.23 (m, 4H). LC/MS: Anal. Calcd. For [M+H]⁺ C51H57F2N9O8S; 994.4. found 994.6.

### Example 60

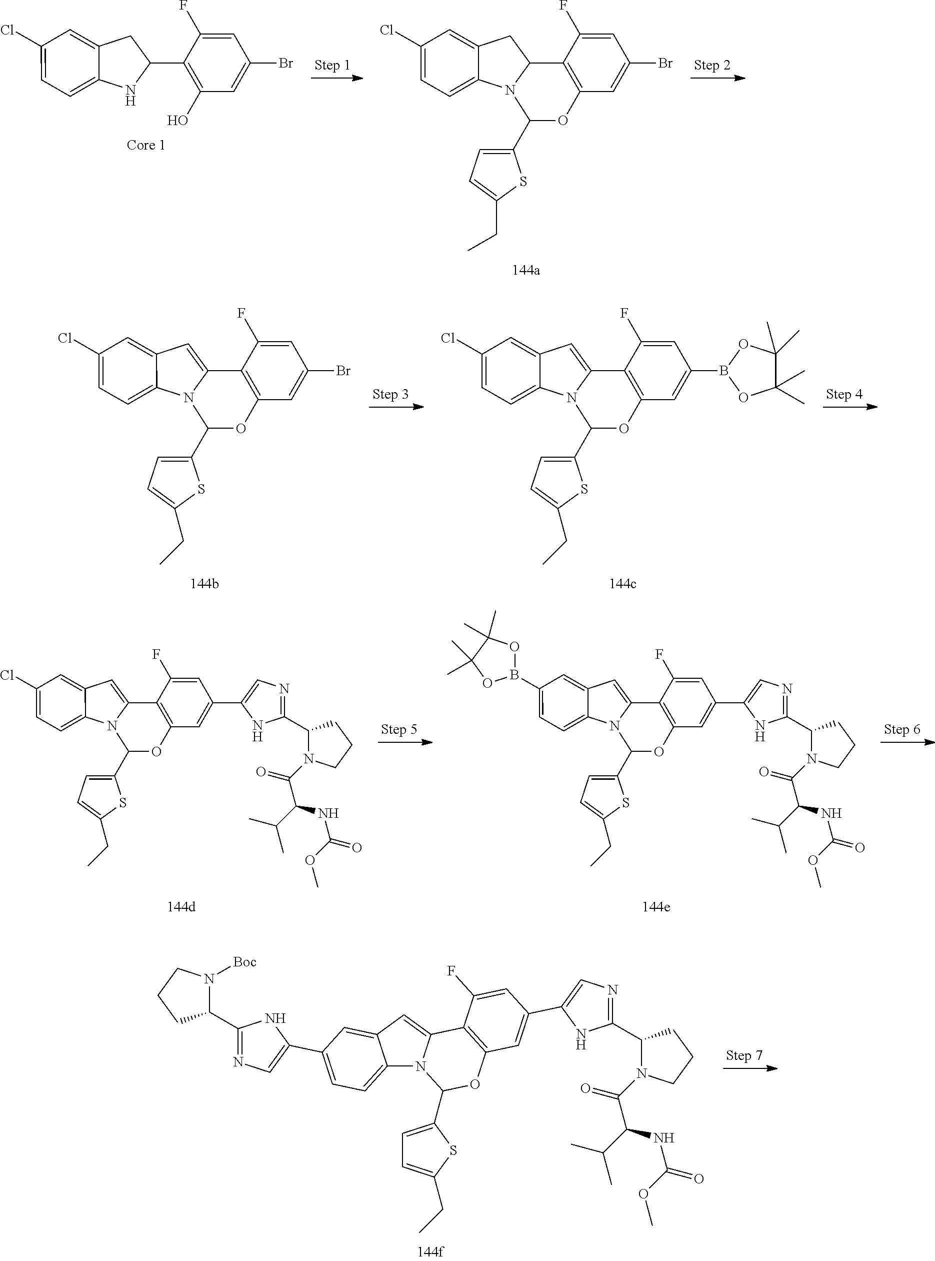

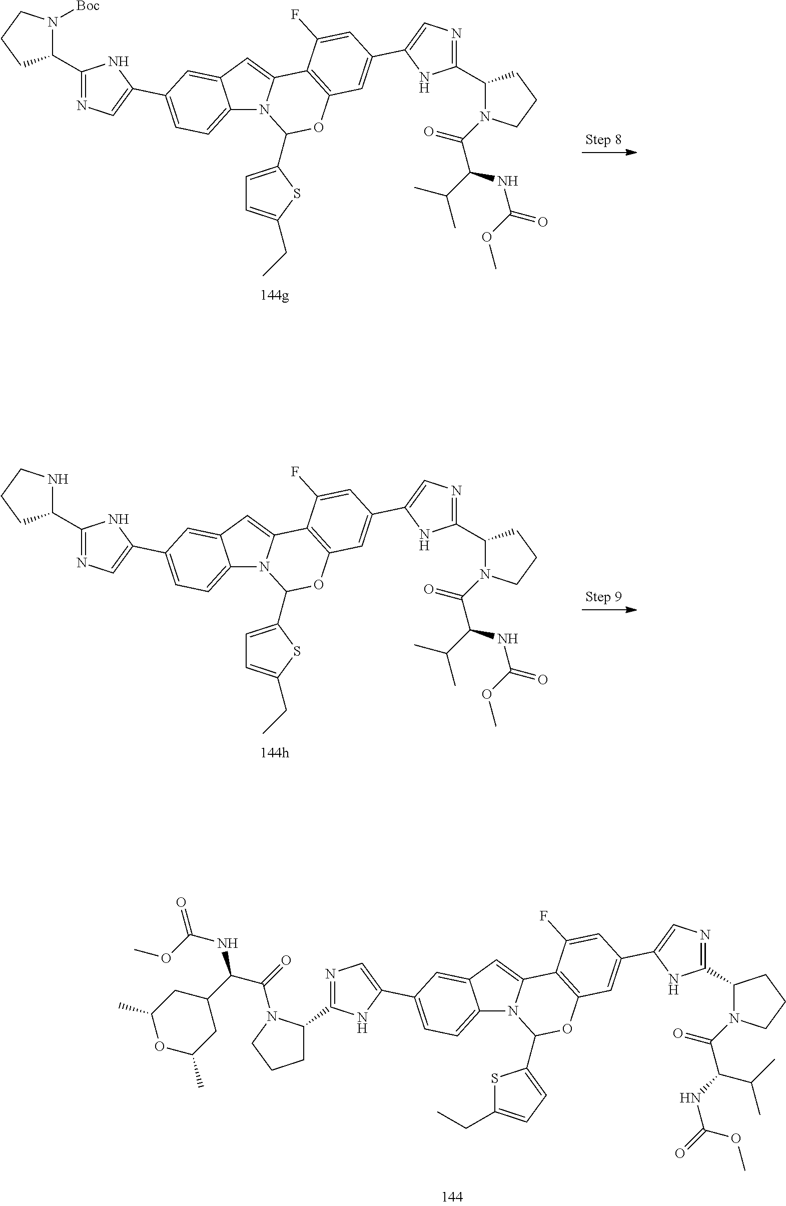 [SMILES: CCc1ccc(C2Oc3cc(-c4cnc([C@@H]5CCCN5C(=O)[C@@H](NC(=O)OC)C(C)C)[nH]4)cc(F)c3-c3cc4cc(-c5cnc([C@@H]6CCCN6C(=O)OC(C)(C)C)[nH]5)ccc4n32)s1 ; CCc1ccc(C2Oc3cc(-c4cnc([C@@H]5CCCN5C(=O)[C@@H](NC(=O)OC)C(C)C)[nH]4)cc(F)c3-c3cc4cc(-c5cnc([C@@H]6CCCN6)[nH]5)ccc4n32)s1 ; CCc1ccc(C2Oc3cc(-c4cnc([C@@H]5CCCN5C(=O)[C@@H](NC(=O)OC)C(C)C)[nH]4)cc(F)c3-c3cc4cc(-c5cnc([C@@H]6CCCN6C(=O)[C@H](NC(=O)OC)C6C[C@@H](C)O[C@@H](C)C6)[nH]5)ccc4n32)s1]

### Step 1

To a solution of core 1 (20 g, 58 mmol) and 5-Ethyl-2-thiophenecarboxaldehyde (12 g, 87 mmol) in MeCN (200 mL) was added TFA (1.98 g, 17 mmol). The mixture was allowed to stir at 20° C. for 1 hour, solid appeared. The solid was collected by filtration, washed with MeCN, dried it to provide 144a (19 g, 70.4% yield).

### Step 2

To the solution of 144a (19 g, 43 mmol) in dry toluene (100 mL) was added DDQ (14.6 g, 65 mmol). After refluxing for 2 hours under N₂, the solvent was removed in vacuo and diluted with EtOAc. The organic layer was washed with saturated Na₂S₂O₃ aqueous and brine, dried over Na₂SO₄, filtered and concentrated in vacuo. The residue obtained was washed with MeOH (20 mL), filtered to provide 144b (17.7 g, 89.3%).

### Step 3

A suspension of 144b (19 g, 41 mmol), BIS(PINACOLATO)DIBORON (11.5 g, 45 mmol), KOAc (12 g, 123 mmol) and Pd(dppf)Cl₂ (3 g, 4.1 mmol) in dioxane (200 mL) was allowed to stir at 90° C. under N₂ atmosphere for 2 hours. The reaction mixture was cooled and concentrated in vacuo, then purified using SiO₂ chromatographed (400 g, Hexane/EtOAc 1% to 20%) to provide 144c (17.7 g, 85.1%).

### Step 4

A suspension of 144c (1 g, 2 mmol), cap 31 (810 mg, 2.2 mmol), Na₂CO₃ (640 mg, 6 mmol) and Pd(dppf)Cl₂ (150 mg, 0.2 mmol) in THF/H₂O (20 mL) was allowed to stir at 90° C. under N₂ atmosphere for about 15 hours. The reaction mixture was concentrated in vacuo and purified using Flash column chromatography on silica gel (20 g, Hexane/EtOAc=20% to DCM:MeOH=2%) to provide 144d (0.9 g, 66.7% yield). LC/MS: Anal. Calcd. For [M+H]⁺ C35H35ClFN5O4S; 676.2. found 676.3.

### Step 5

To a mixture of 144d (0.9 g, 1.3 mmol), BIS(PINACOLATO)DIBORON (400 mg, 1.5 mmol), KOAc (400 mg, 3.9 mmol), Pd₂(dba)₃ (110 mg, 0.13 mmol), X-Phos (62 mg, 0.13 mmol) degassed and sealed under N₂ was added dry dioxane (10 mL), following further N₂ purging. The mixture was allowed to stir at 130° C. for about 15 hours. The mixture was concentrated in vacuo, added ethyl acetate and water. The organic layer was separated, dried over anhydrous sodium sulfonate, concentrated in vacuo. The residue obtained was purified using Flash column chromatography on silica gel (20 g, DCM/MeOH 0% to 2%) to provide 144e (0.9 g, 82.5% yield). LC/MS: Anal. Calcd. For [M+H] C41H47BFN5O6S; 768.3. found 768.4. In case of compound 511 and 533, BIS(TRI-TERT-BUTYLPHOSPHINE)PALLADIUM(0) (0.1 eq) was used instead of Pd₂(dba)₃.

### Step 6

A suspension of 144e (0.9 g, 1.3 mmol), cap 31a (0.5 g, 1.4 mmol), Na₂CO₃ (400 mg, 3.9 mmol) and Pd(dppf)Cl₂ (100 mg, 0.13 mmol) in THF/H₂O (20 mL) was allowed to stir at 90° C. under N₂ for about 15 hours. The reaction mixture was concentrated in vacuo and purified using Flash column chromatography on silica gel (10 g, DCM/MeOH 1% to 3%) to provide 144f (0.8 g, 70.7% yield). LC/MS: Anal. Calcd. For [M+H]⁺ C47H53FN8O6S; 877.4. found 877.5.

### Step 7

The two diastereo isomers of 144f (1 g) were separated by SFC by using the following conditions.

Column: OZ-H

Solvent: ETOH (0.05% DEA)

Flow rate: 2 mL/min

Wavelength: 220 nm

144g (305 mg, 61% yield). LC/MS: Anal. Calcd. For [M+H] C47H53FN8O6S: 877.4. found 877.4.

### Step 8

To a solution of 144g (0.3 g, 0.34 mmol) in dioxane (2 mL) was added HCl/dioxane (2 mL, 4M) and stirred at ambient temperature for 2 hours, then concentrated in vacuo and dried under high vacuum to provide 144h (265 mg, 100% yield). LC/MS: Anal. Calcd. For [M+H]⁺ C42H45FN8O4S; 777.3. found 777.6.

### Step 9

To a mixture of 144h (150 mg, 0.19 mmol), cap 4 (47 mg, 0.19 mmol) and DIPEA (0.5 mL) in DMF (5 mL was added HATU (74 mg, 0.19 mmol). The resulting mixture was allowed to stir at 25° C. for 2 hours. The solution was purified using Pre-HPLC to provide 144 (75 mg, 39.3%). ¹H NMR (MeOD) δ: 8.02 (br. 1H), 7.87 (br., 1H), 7.80-7.83 (m, 1H), 7.75-7.79 (m, 1H), 7.50 (m, 2H), 7.33-7.39 (m, 1H), 7.27-7.32 (m, 1H), 7.14-7.20 (m, 1H), 6.51-6.56 (m, 1H), 6.46-6.51 (m, 1H), 5.14-5.26 (m, 2H), 4.16-4.26 (m, 2H), 4.03-4.14 (m, 2H), 3.78-3.91 (m, 2H), 3.63 (d, J=1.57 Hz, 6H), 3.35-3.48 (m, 3H), 2.67 (d, J=7.43 Hz, 2H), 2.46-2.59 (m, 2H), 1.94-2.30 (m, 8H), 1.51-1.60 (m, 1H), 1.25-1.33 (m, 1H), 1.13 (t, J=7.43 Hz, 4H), 1.03-1.10 (m, 6H), 0.83-0.99 (m, 7H). LC/MS: Anal. Calcd. For [M+H]⁺ C53H62FN9O8S; 1004.4. found 1004.5.

The following compounds of the present invention were made using the methods described in the Example above and substituting the appropriate reactants or reagents.

| | | | Observed |
|---|---|---|---|
| ID | Structures | Isomer | [M + H]+ |
| 39 40 41 42 | 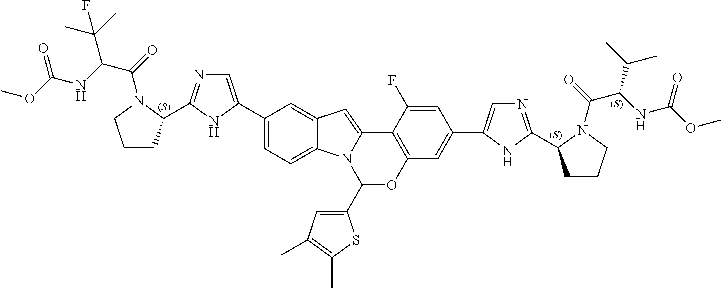 | Isomer 1 Isomer 2 Isomer 3 Isomer 4 | 952.4 952.6 952.6 952.6 |
| 75 76 77 78 | 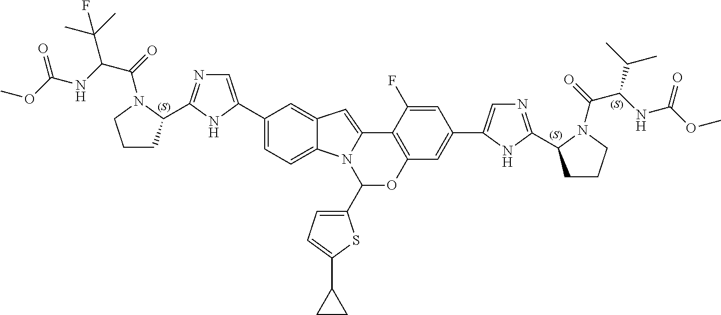 | Isomer 1 Isomer 2 Isomer 3 Isomer 4 | 964.3 964.3 964.6 964.6 |
| 94 95 96 | 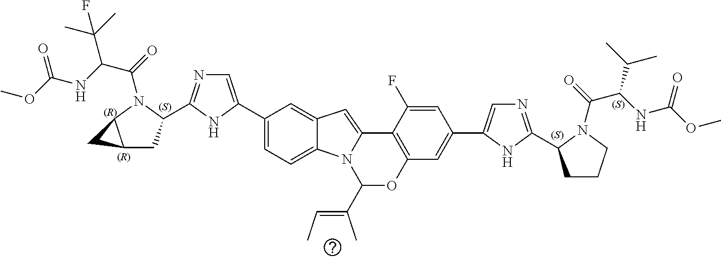 | Isomer 1 Isomer 2 Isomer 3 | 976.6 976.4 976.6 |
| 109 | 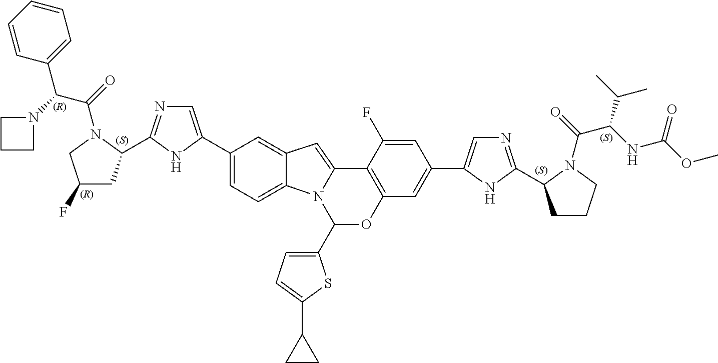 | racemic | 980.6 |
| 112 113 114 115 | 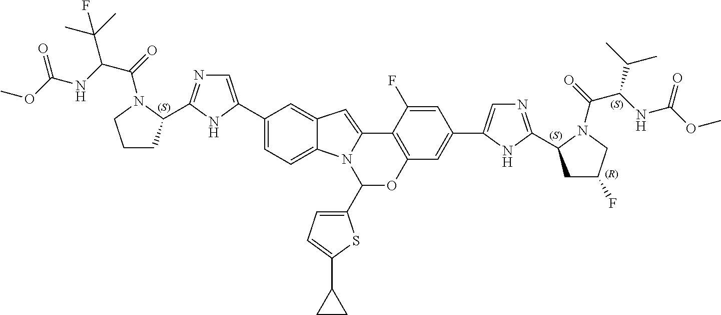 | Isomer 1 Isomer 2 Isomer 3 Isomer 4 | 982.6 982.4 982.4 982.6 |
| 126 | 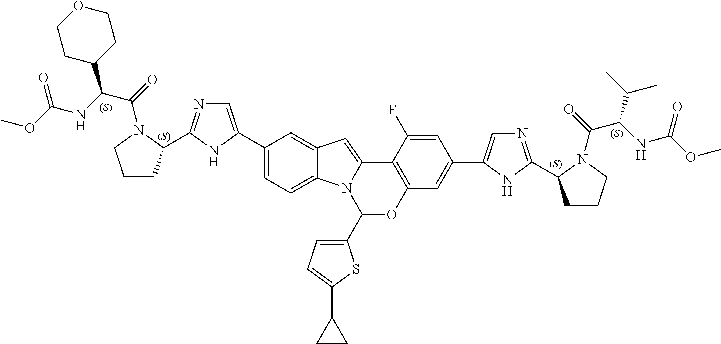 | Isomer 1 | 988.5 |
| 129 130 131 | 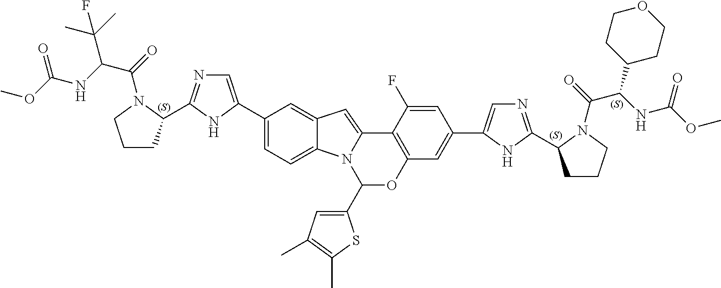 | Isomer 2 Isomer 3 Isomer 4 | 994.3 994.6 994.4 |
| 134 135 136 137 138 139 140 141 | 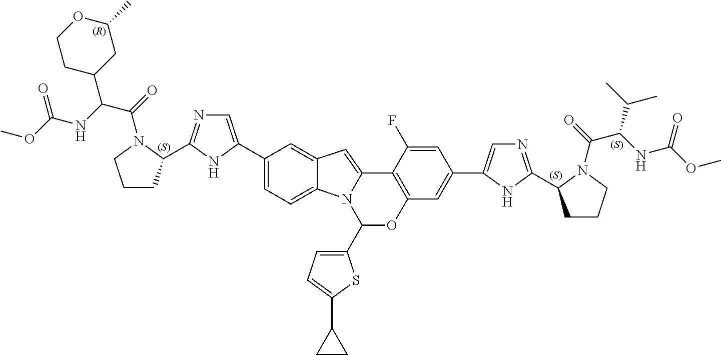 | Isomer 1 Isomer 2 Isomer 3 Isomer 4 Isomer 5 Isomer 6 Isomer 7 Isomer 8 | 1002.6 1002.6 1002.4 1002.6 1002.4 1002.8 1002.6 1002.6 |
| 142 143 145 | 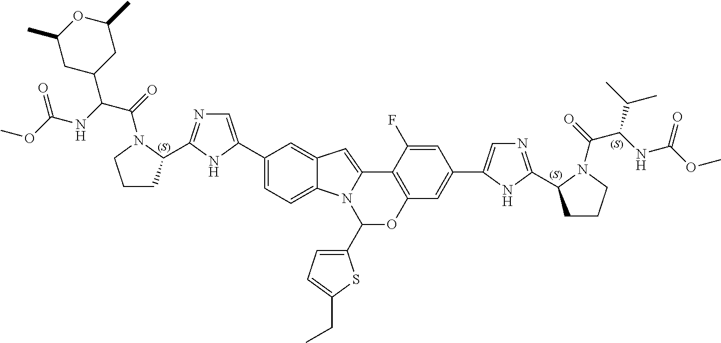 | Isomer 1 Isomer 2 Isomer 4 | 1004.4 1004.6 1004.8 |
| 146 147 148 149 152 153 | 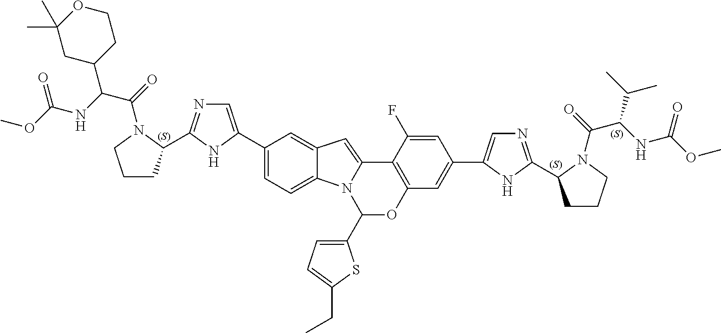 | Isomer 1 Isomer 2 Isomer 3 Isomer 4 Isomer 5 Isomer 6 | 1004.6 1004.8 1004.6 1004.6 1004.4 1004.6 |
| 166 167 | 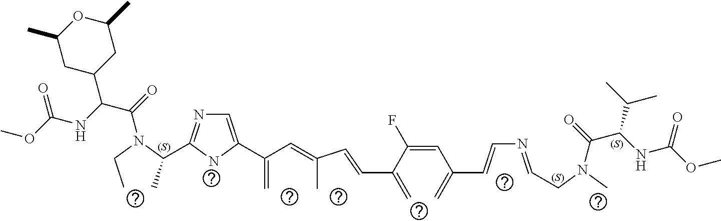 | Isomer 7 Isomer 8 | 1004.6 1004.6 |
| 172 173 174 175 | 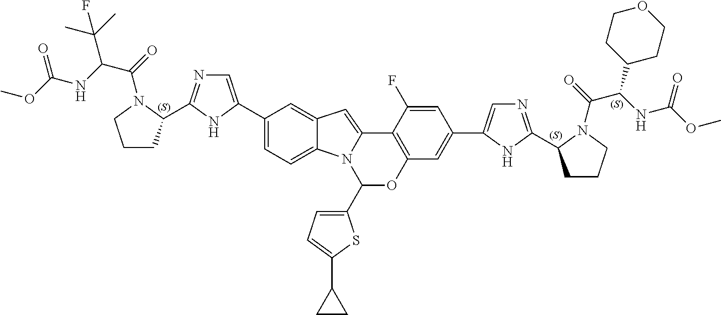 | Isomer 1 Isomer 2 Isomer 3 Isomer 4 | 1006.8 1006.6 1006.6 1006.6 |
| 176 177 178 179 | 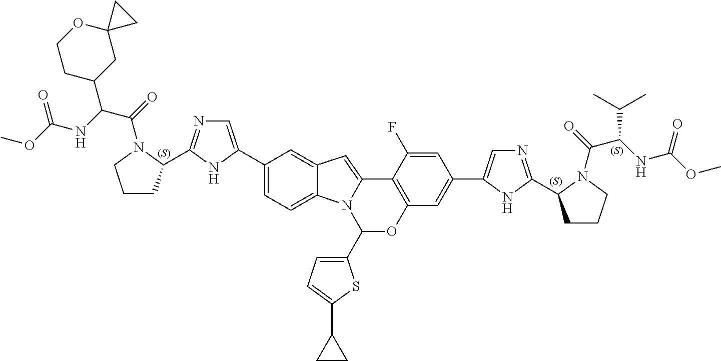 | Isomer 1 Isomer 2 Isomer 3 Isomer 4 | 1014.6 1014.6 1014.6 1014.6 |
| 180 181 | 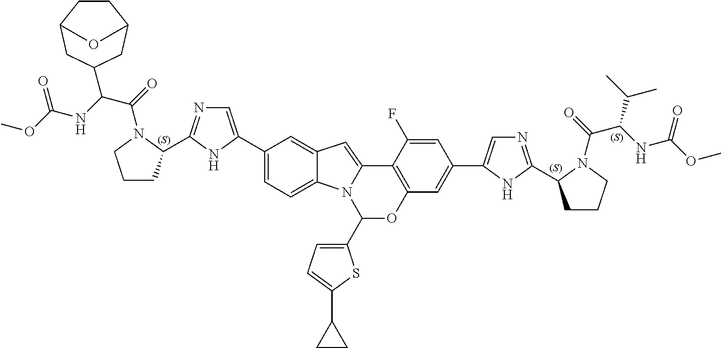 | Isomer 1 Isomer 2 | 1014.6 1014.6 |
| 182 183 184 185 | 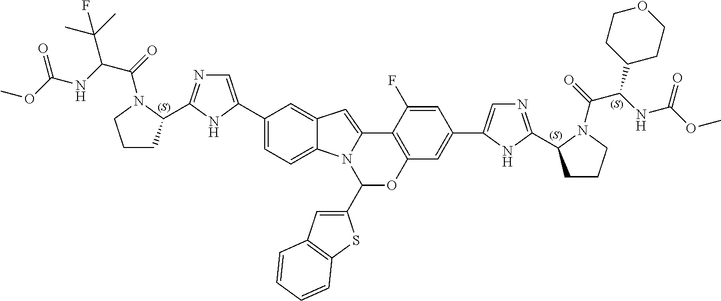 | Isomer 1 Isomer 2 Isomer 3 Isomer 4 | 1016.3 1016.6 1016.6 1016.4 |
| 191 192 193 | 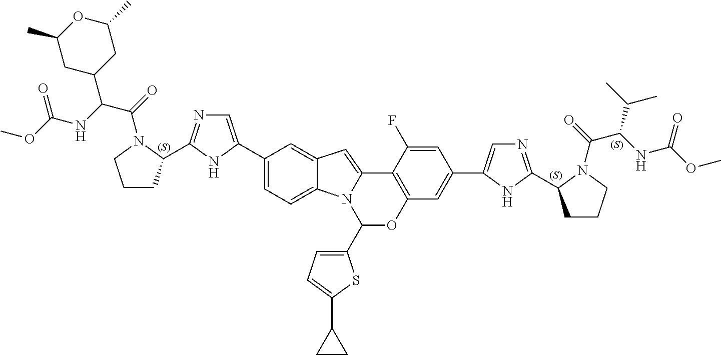 | Isomer 2 Isomer 3 Isomer 4 | 1016.5 1016.4 1016.4 |
| 194 | 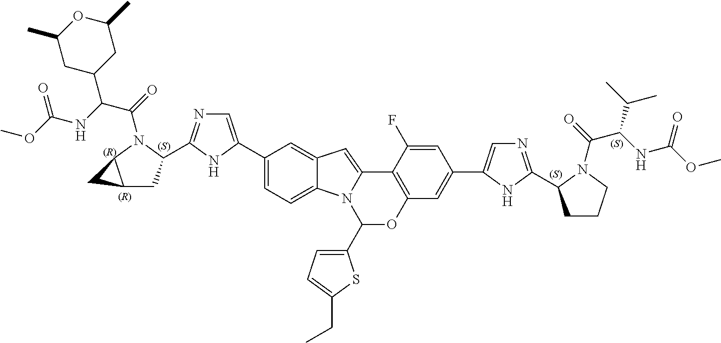 | Isomer 1 | 1016.6 |
| 196 197 198 199 200 201 202 203 | 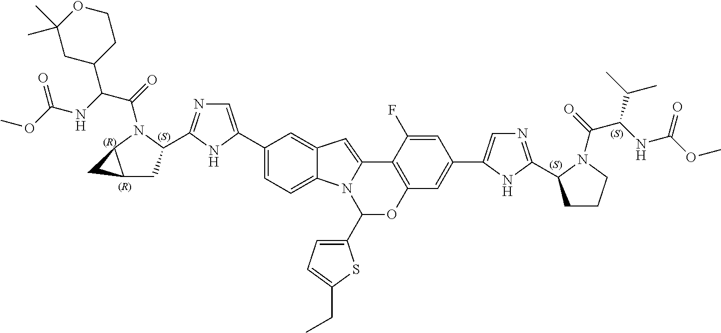 | Isomer 1 Isomer 2 Isomer 3 Isomer 4 Isomer 5 Isomer 6 Isomer 7 Isomer 8 | 1016.6 1016.6 1016.8 1016.6 1016.7 1016.5 1016.6 1016.6 |
| 204 205 | 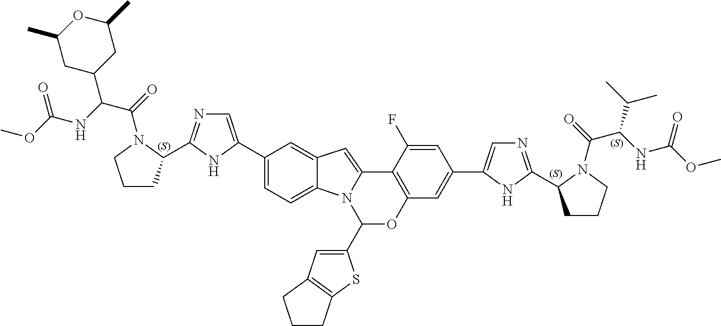 | Isomer 1 Isomer 2 | 1016.7 1016.7 |
| 206 207 209 | 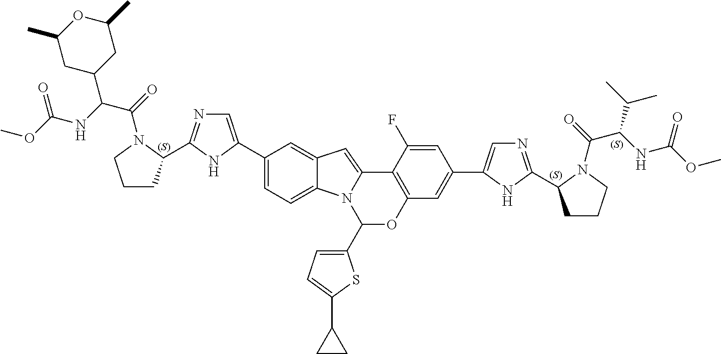 | Isomer 1 Isomer 2 Isomer 4 | /1016.4 1016.7 1016.9 |
| 220 221 222 223 224 225 | 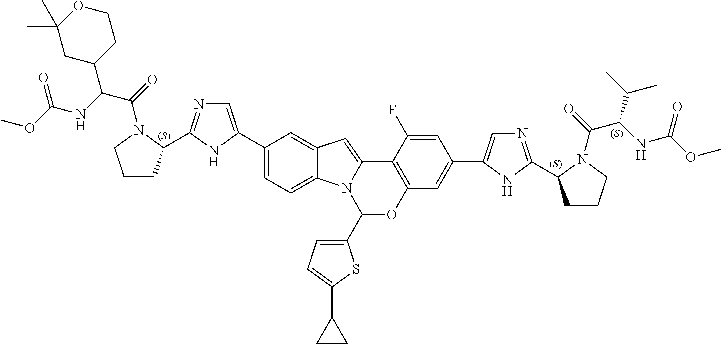 | Isomer 3 Isomer 4 Isomer 5 Isomer 6 Isomer 7 Isomer 8 | 1016.6 1016.6 1016.6 1016.2 1016.6 1016.2 |
| 226 227 228 229 | 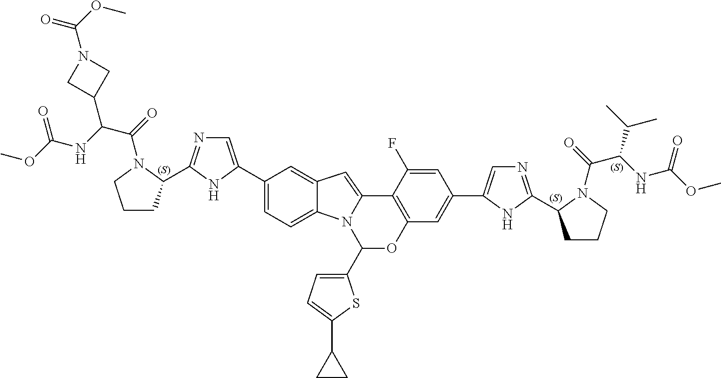 | Isomer 1 Isomer 2 Isomer 3 Isomer 4 | 1017.6 1017.6 1017.2 1017.8 |
| 230 231 | 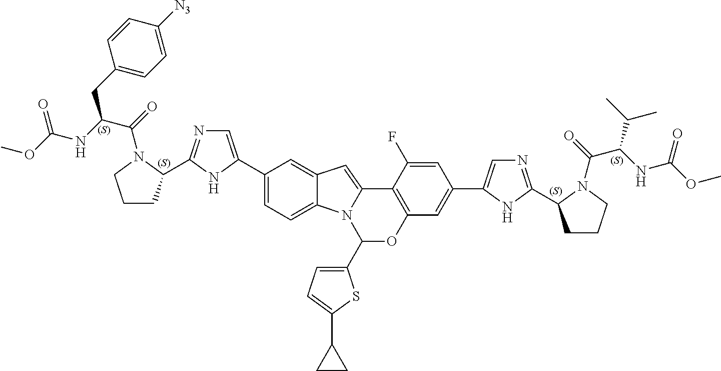 | Isomer 1 Isomer 2 | 1017.6 1017.6 |
| 232 233 235 | 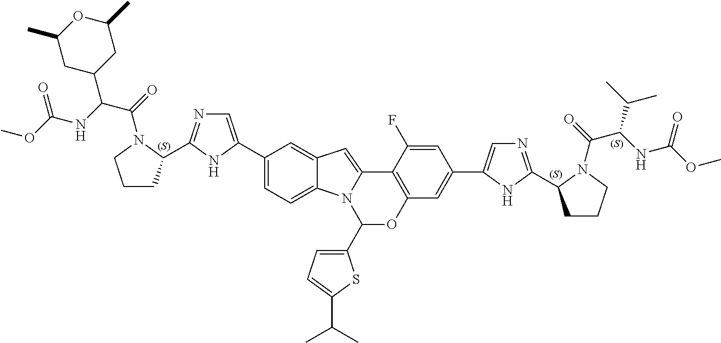 | Isomer 1 Isomer 2 Isomer 4 | 1018.6 1018.6 1018.2 |
| 236 237 238 239 242 243 | 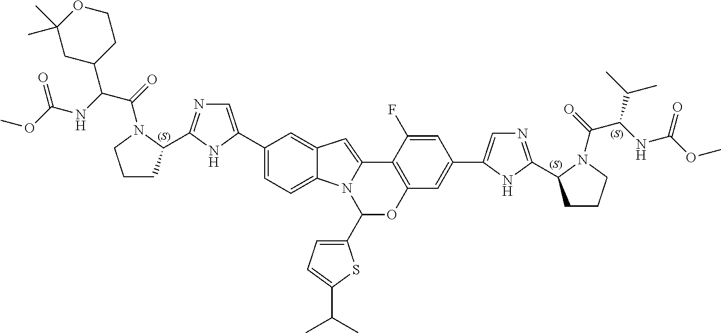 | Isomer 1 Isomer 2 Isomer 3 Isomer 4 Isomer 7 Isomer 8 | 1018.6 1018.8 1018.8 1018.6 1018.4 1018.6 |
| 247 | 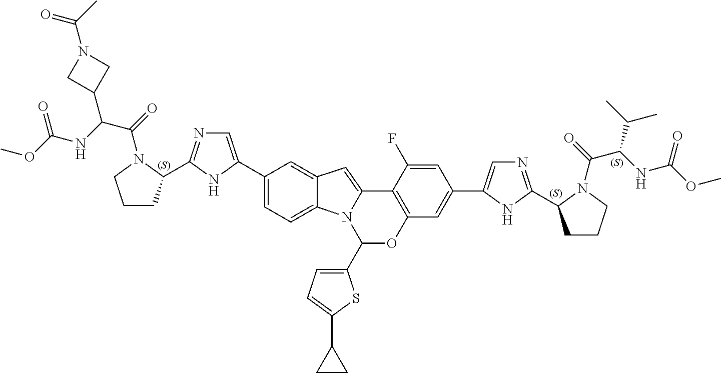 | Isomer 1 | 1019.6 |
| 252 253 | 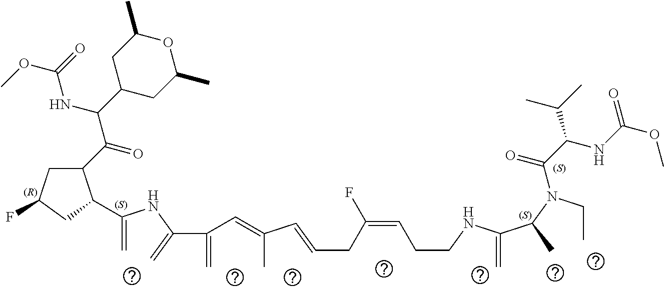 | Isomer 1 Isomer 2 | 1022.6 1022.2 |
| 254 255 256 257 258 259 260 261 | 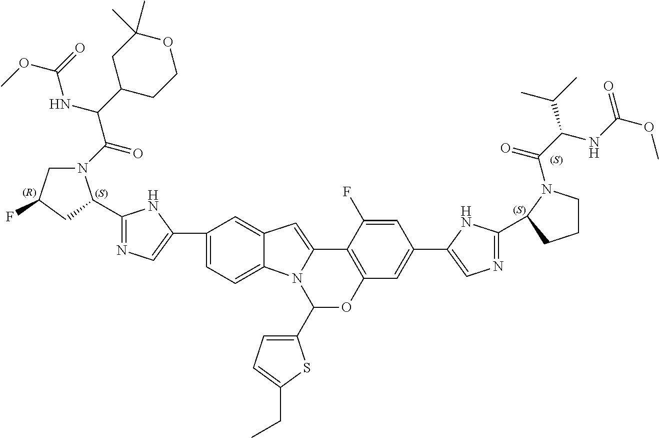 | Isomer 1 Isomer 2 Isomer 3 Isomer 4 Isomer 5 Isomer 6 Isomer 7 Isomer 8 | 1022.6 1022.4 1022.4 1022.6 1022.6 1022.5 1022.6 1022.6 |
| 265 266 | 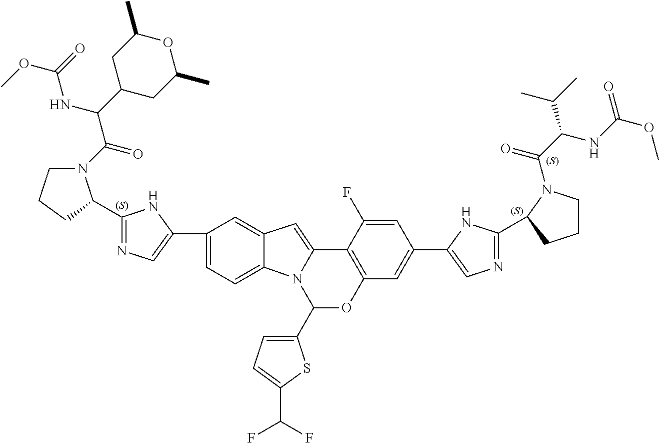 | Isomer 1 Isomer 2 | 1026.4 1026.4 |
| 267 268 269 270 | 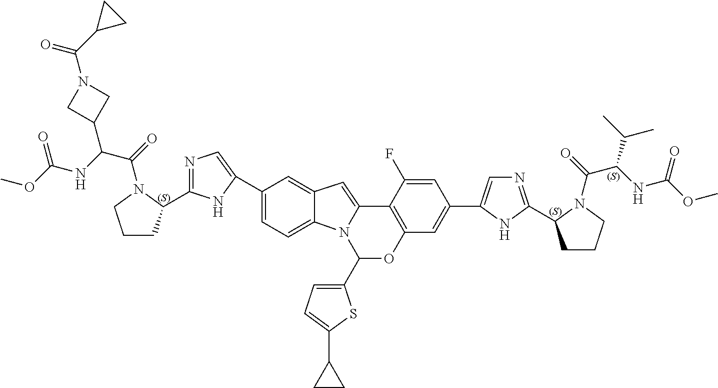 | Isomer 1 Isomer 2 Isomer 3 Isomer 4 | 1027.6 1027.6 1027.6 1027.8 |
| 271 272 273 274 | 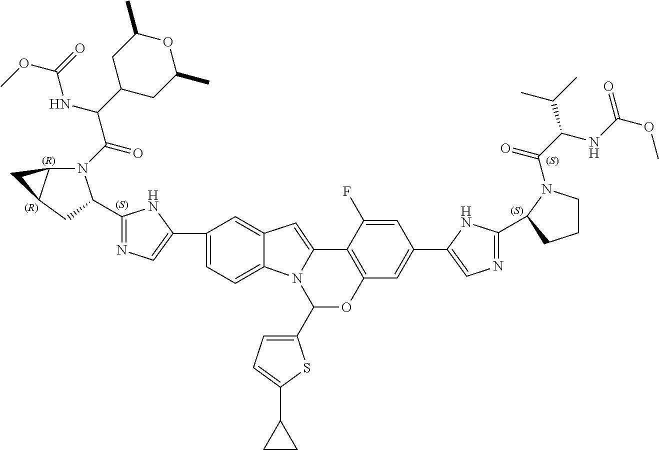 | Isomer 1 Isomer 2 Isomer 3 Isomer 4 | 1028.6 1028.8 1028.6 1028.6 |
| 276 277 278 | 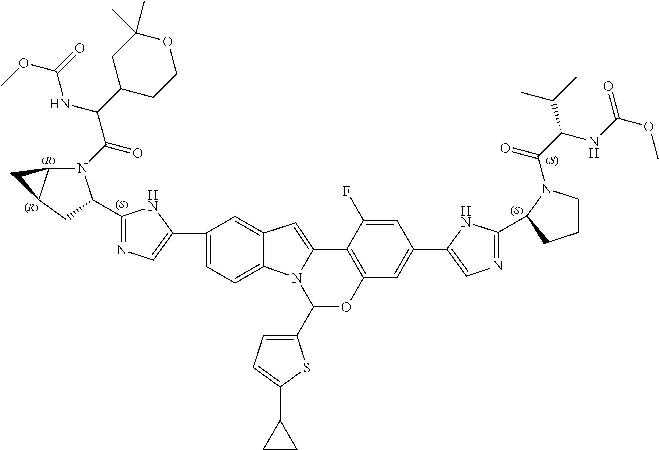 | Isomer 2 Isomer 3 Isomer 4 | 1028.6 1028.6 1028.6 |
| 281 282 | 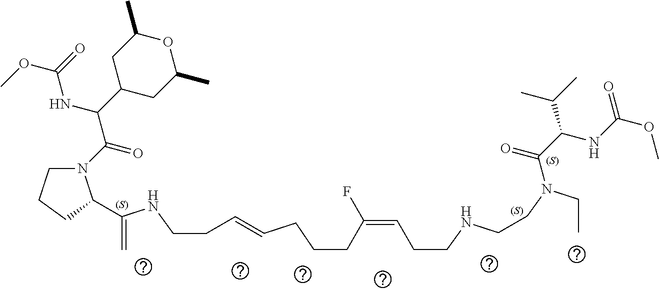 | Isomer 1 Isomer 2 | 1030.6 1030.6 |
| 283 | 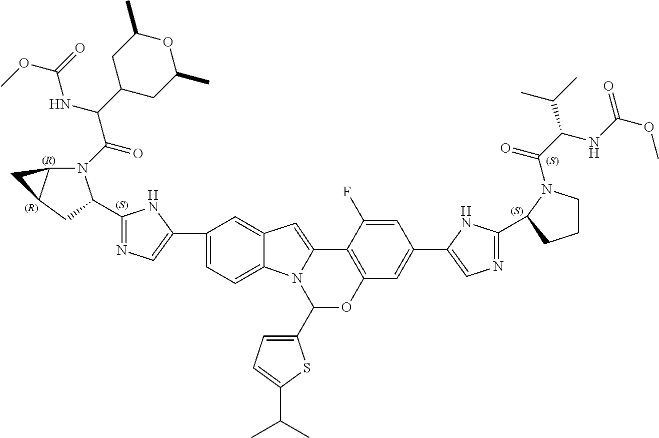 | Isomer 1 | 1030.6 |
| 285 286 | 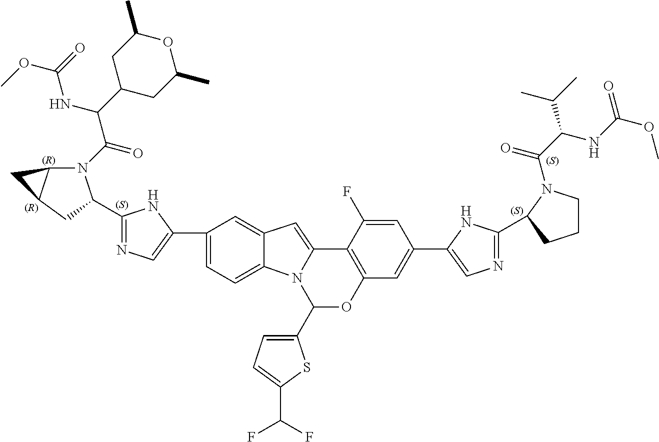 | Isomer 1 Isomer 2 | 1039.0 1038.4 |
| 287 288 289 290 | 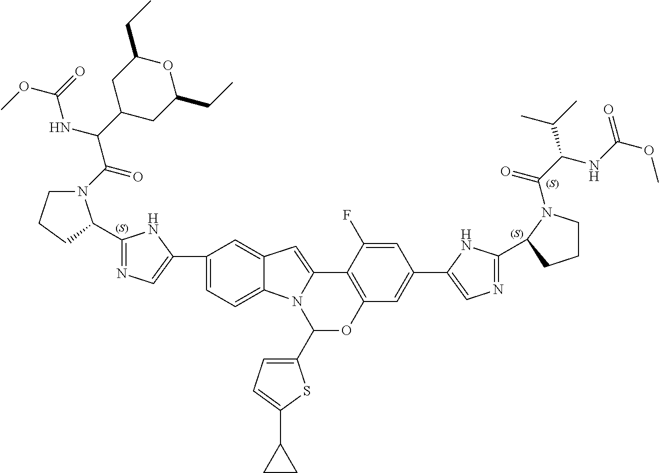 | Isomer 1 Isomer 2 Isomer 3 Isomer 4 | 1044.6 1044.6 1044.6 1044.6 |
| 314 | 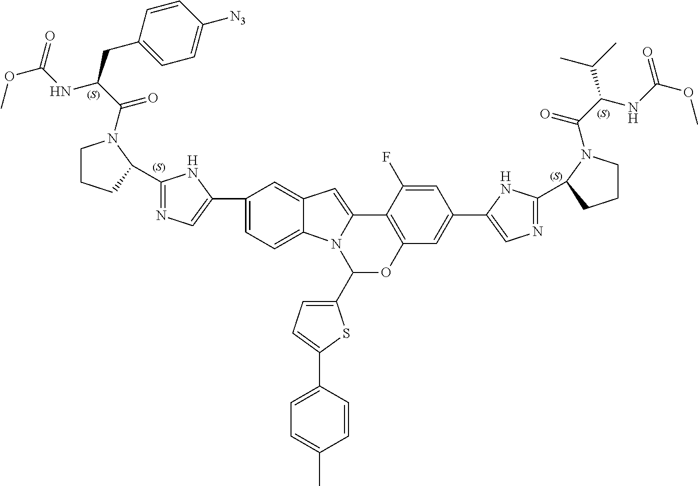 | racemic | 1197.4 |
| 334 335 | 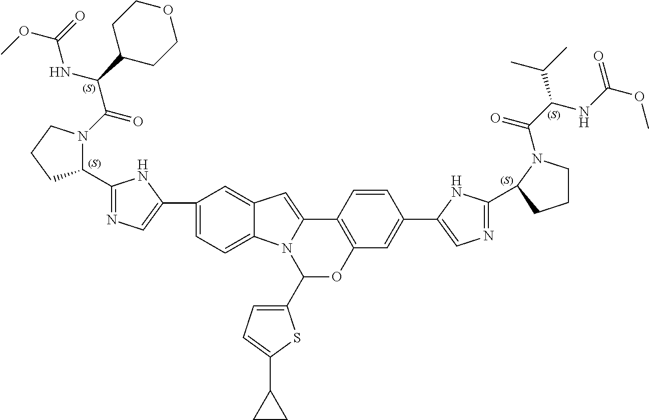 | Isomer 1 Isomer 2 | 970.4 970.4 |
| 336 337 | 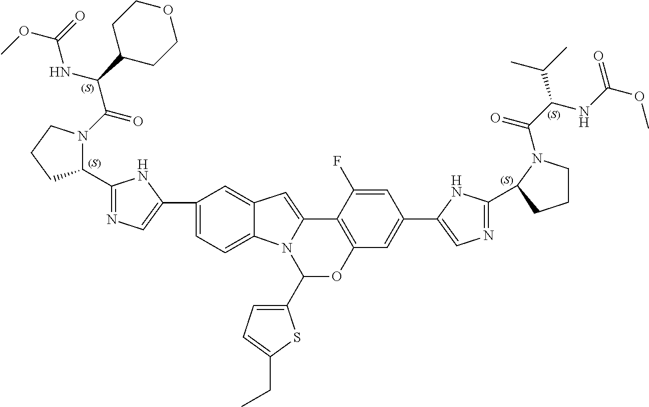 | Isomer 1 Isomer 2 | 976.4 976.4 |
| 338 339 | 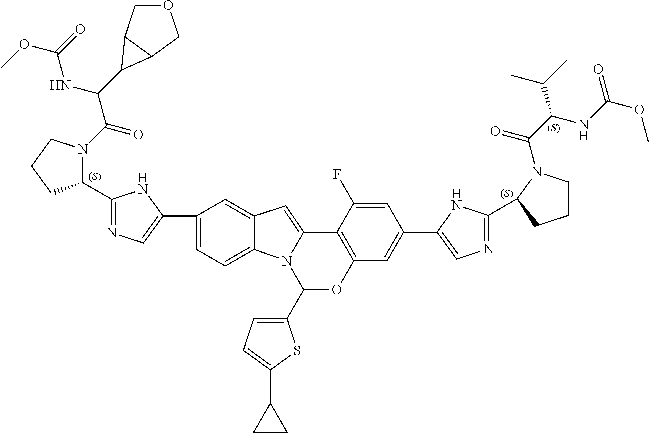 | Isomer 1 Isomer 2 | 986.4 986.4 |
| 340 341 342 343 | 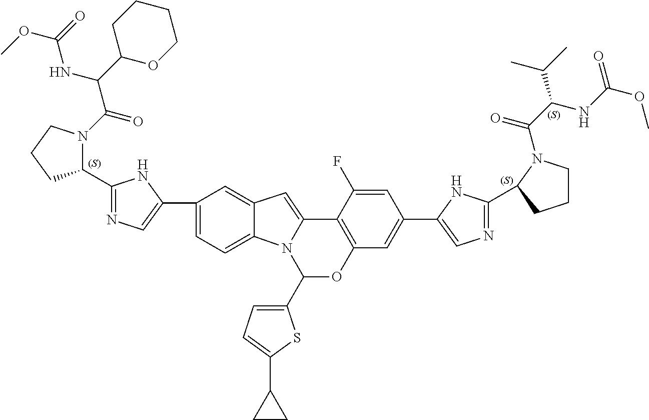 | Isomer 1 Isomer 2 Isomer 3 Isomer 4 | 988.4 988.4 988.4 988.4 |
| 344 345 | 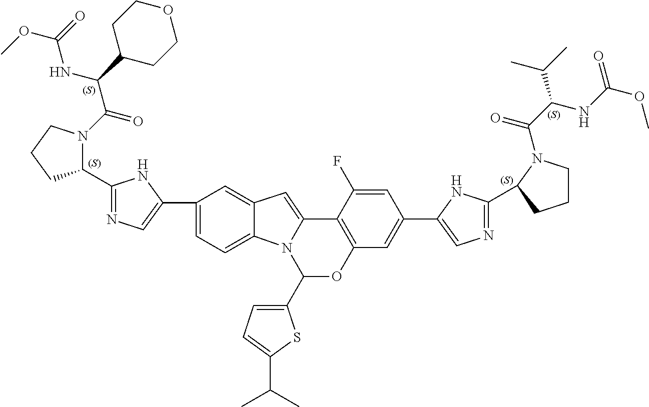 | Isomer 1 Isomer 2 | 990.4 990.4 |
| 346 347 | 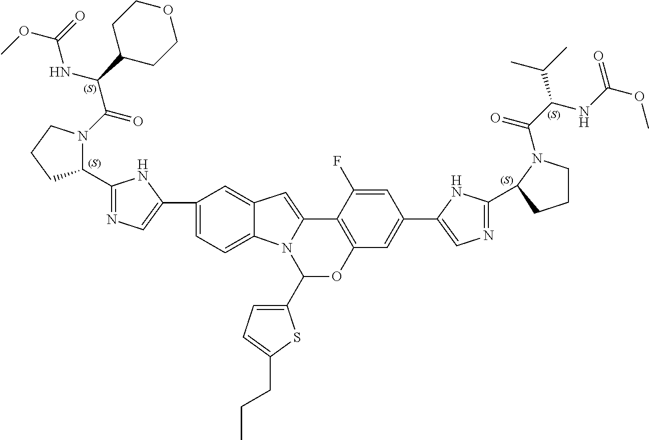 | Isomer 1 Isomer 2 | 990.4 990.4 |
| 348 349 | 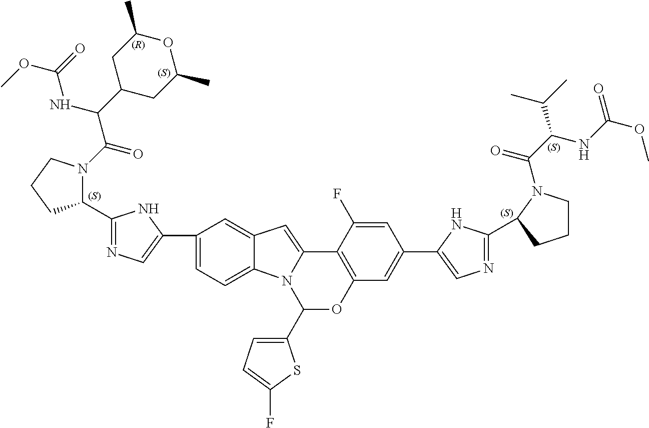 | Isomer 1 Isomer 2 | 994.4 994.4 |
| 350 351 | 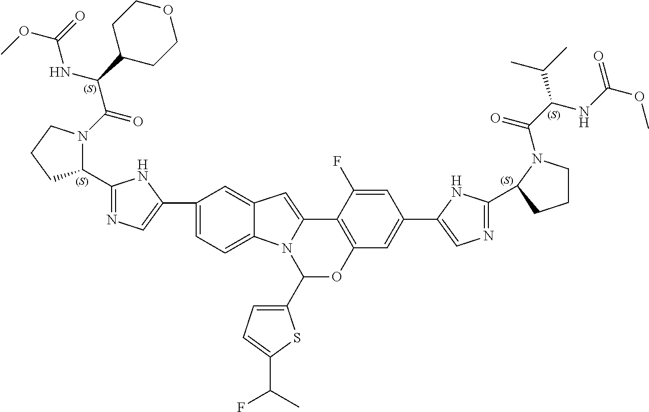 | Isomer 1 Isomer 2 | 998.4 998.4 |
| 352 353 | 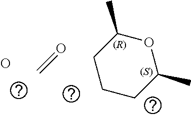 | Isomer 1 Isomer 2 | 998.4 998.4 |
| 354 355 | 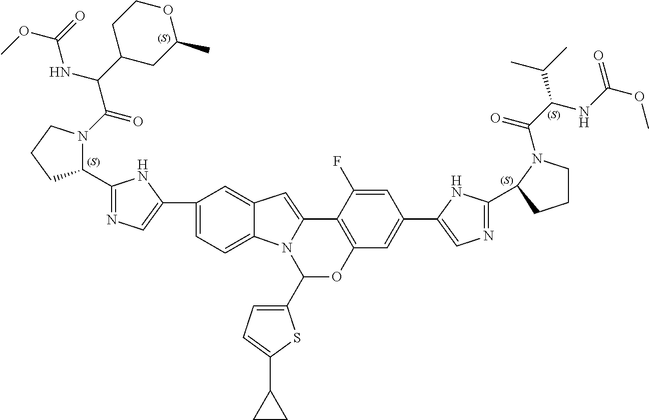 | Isomer 1 Isomer 2 | 1002.4 1002.4 |
| 356 357 | 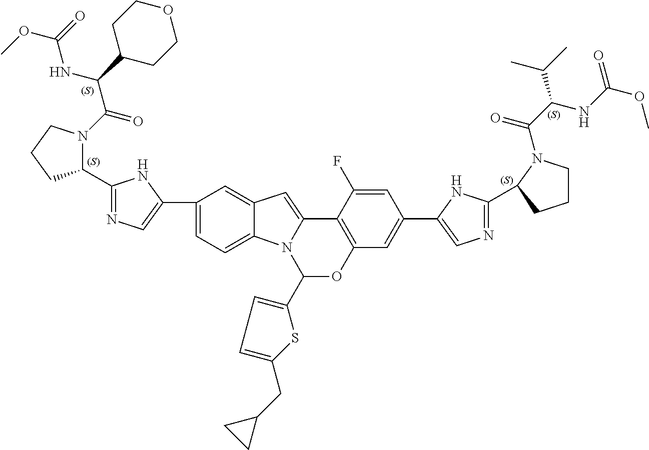 | Isomer 1 Isomer 2 | 1002.4 1002.4 |
| 360 361 | 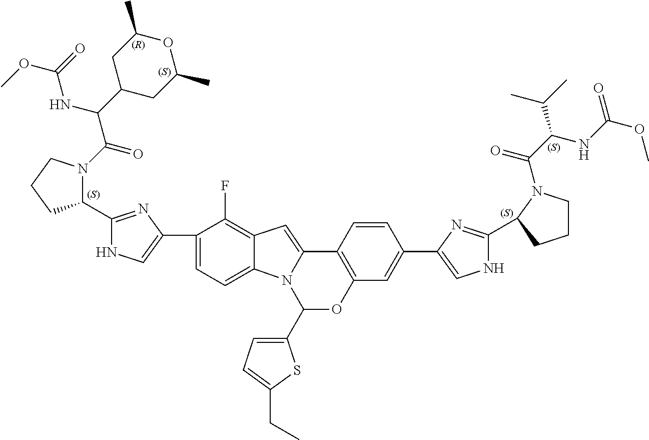 | Isomer 1 Isomer 2 | 1004.4 1004.4 |
| 364 | 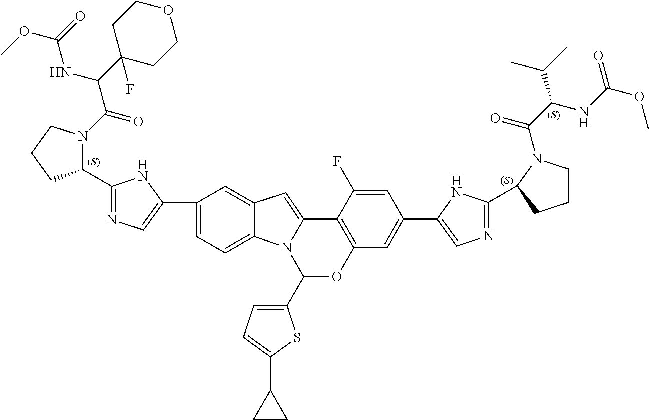 | Isomer 1 | 1006.4 |
| 369 370 | 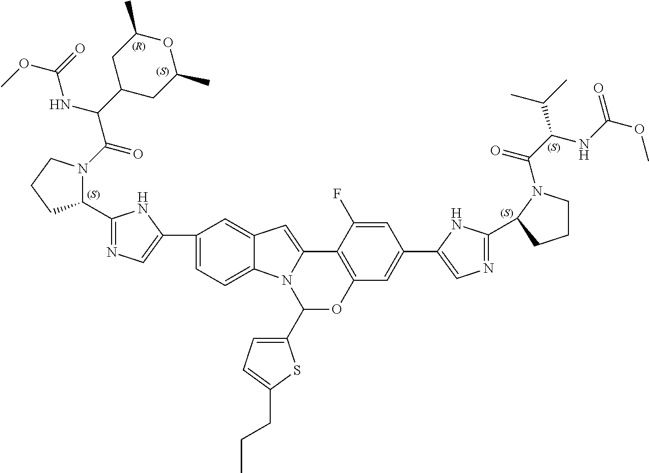 | Isomer 1 Isomer 2 | 1018.5 1018.5 |
| 371 372 | 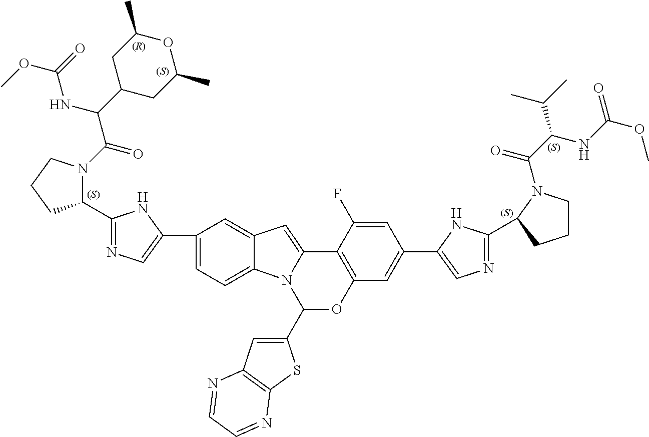 | Isomer 1 Isomer 2 | 1028.4 1028.4 |
| 377 378 | 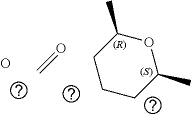 | Isomer 1 Isomer 2 | 1030.5 1030.5 |
| 401 402 | 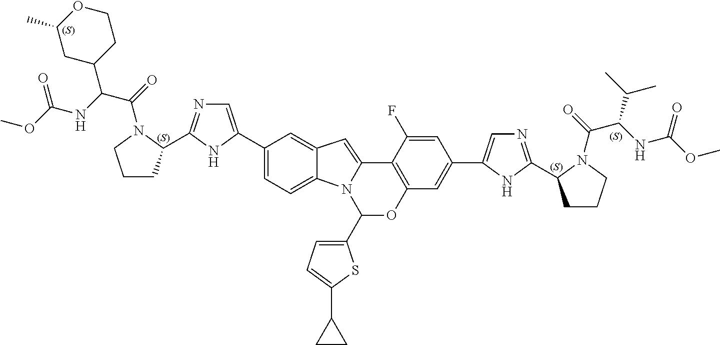 | Isomer 1 Isomer 2 | 1002.1 1002.1 |
| 403 404 | 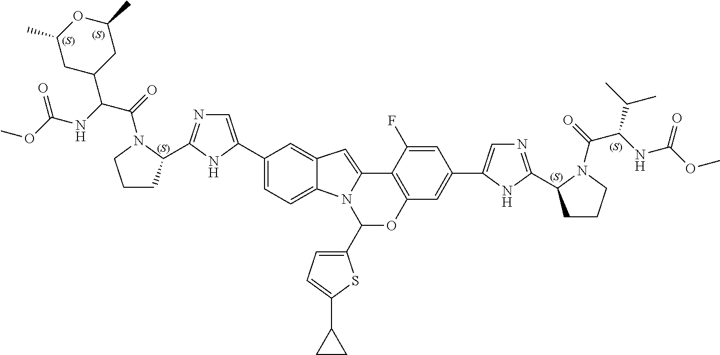 | Isomer 1 Isomer 2 | 1016.2 1016.2 |
| 405 | 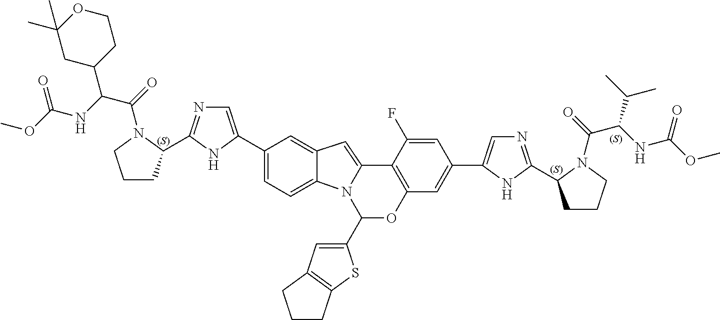 | Isomer 1 | 1016.2 |
| 407 408 409 410 | 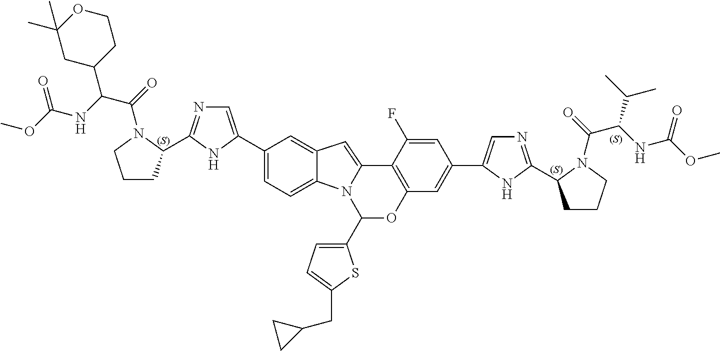 | Isomer 1 Isomer 2 Isomer 3 Isomer 4 | 1030.2 1030.2 1030.2 1030.2 |
| 411 412 | 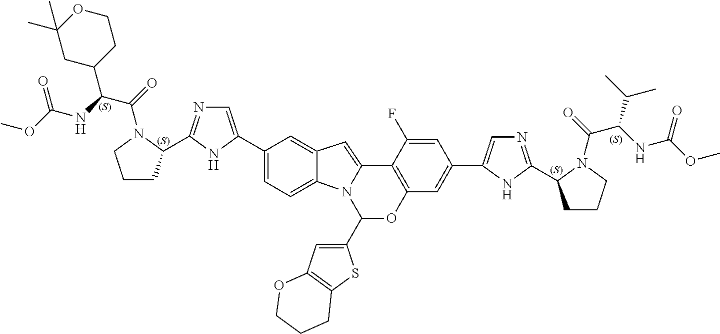 | Isomer 1 Isomer 2 | 1032.2 1032.2 |
| 420 421 | 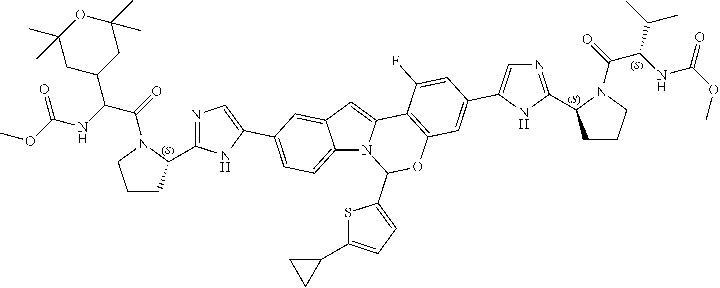 | Isomer 1 Isomer 2 | 1044.2 1044.2 |
| 424 425 | 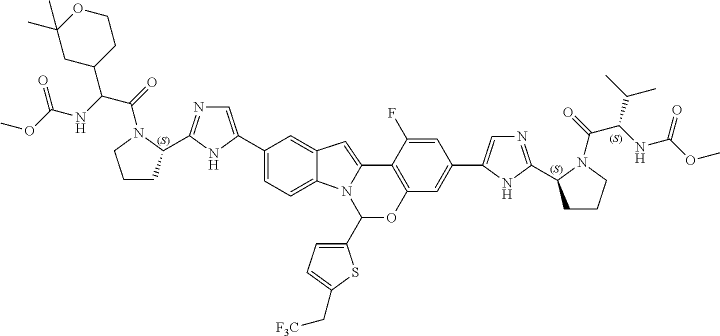 | Isomer 1 Isomer 2 | 1058.1 1058.1 |
| 511 512 513 | 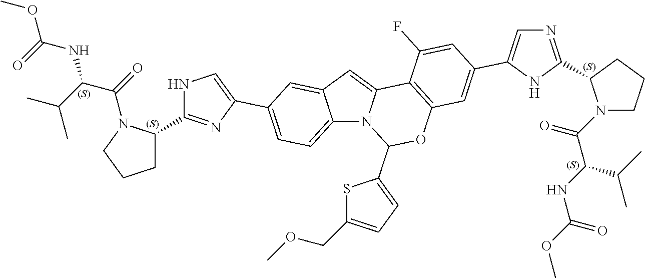 | Isomer 1 Isomer 2 Racemic | 951.17 951.12 951.06 |
| 517 518 519 | 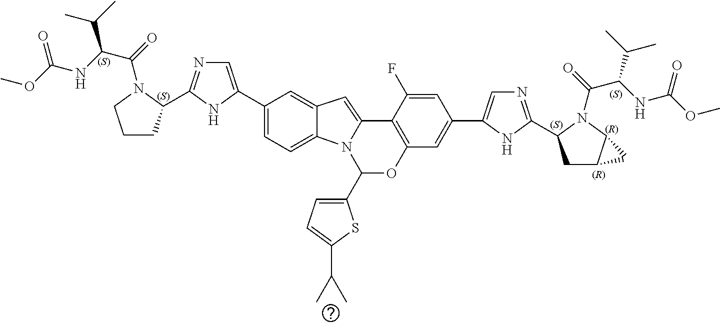 | Racemic Isomer 1 Isomer 2 | 958.84 959.13 959.22 |
| 520 521 522 | 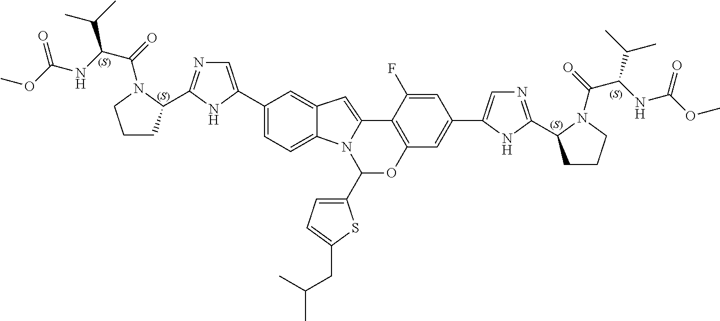 | Racemic Isomer 1 Isomer 2 | 962.97 963.11 963.10 |
| 528 529 530 | 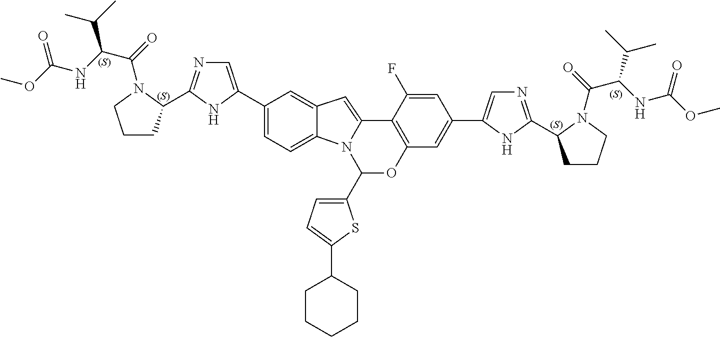 | Isomer 2 Isomer 1 Racemic | 988.93 988.94 989.35 |
| 531 | 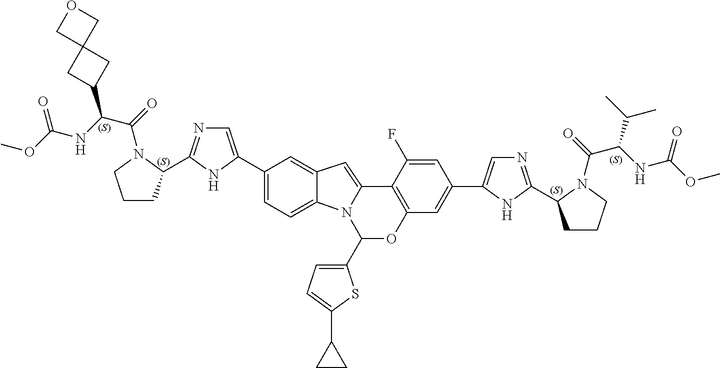 | Racemic | 1000.81 |
| 532 | 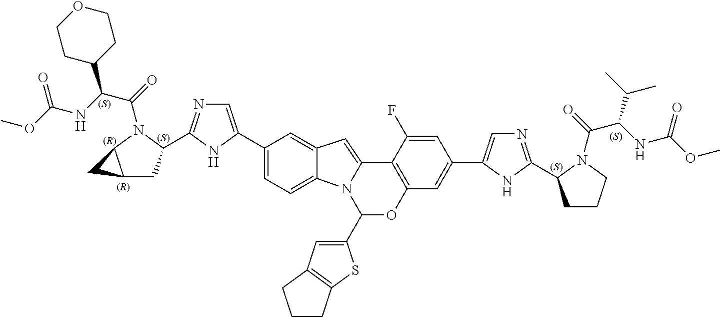 | Racemic | 1001.14 |
| 533 534 535 | 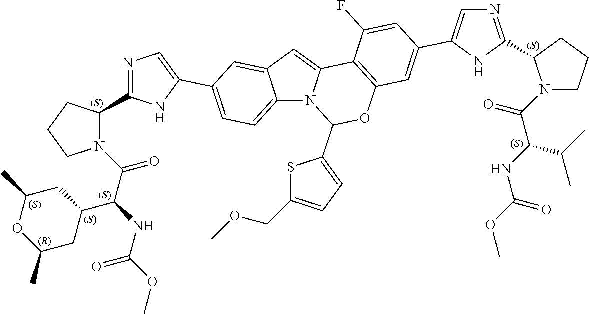 | Isomer 1 Isomer 2 Racemic | 1021.13 1022.32 1021.04 |
| 536 537 538 | 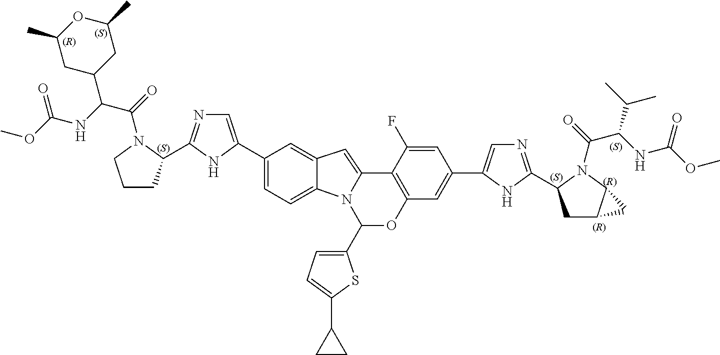 | Isomer 2 Isomer 1 Racemic | 1029.20 1029.82 1029.06 |
| 543 544 | 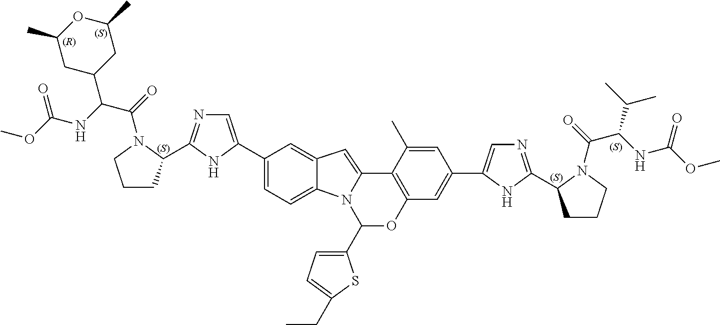 | Isomer 1 Isomer 2 | 1001.33 1001.42 |
| 545 546 | 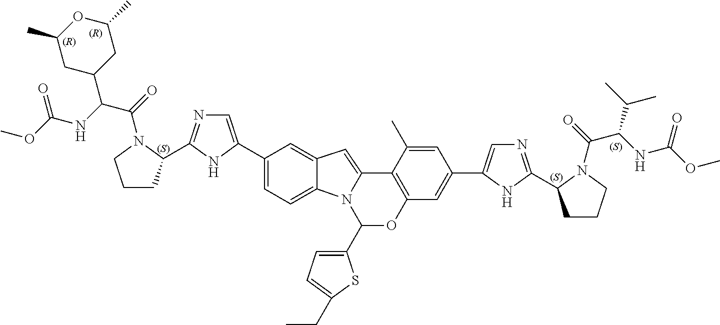 | Isomer 1 Isomer 2 | 1001.41 1001.48 |
| 547 | 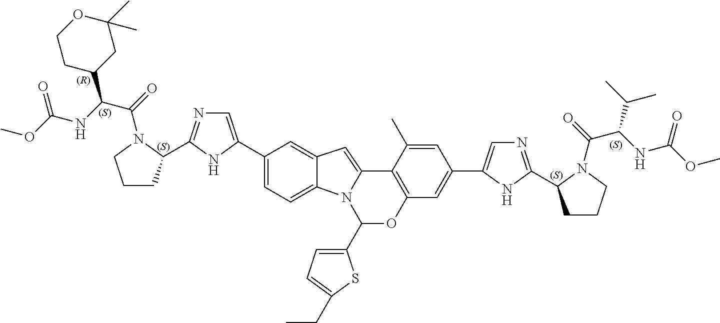 | Isomer 1 | 1001.38 |
| 554 555 556 557 558 | 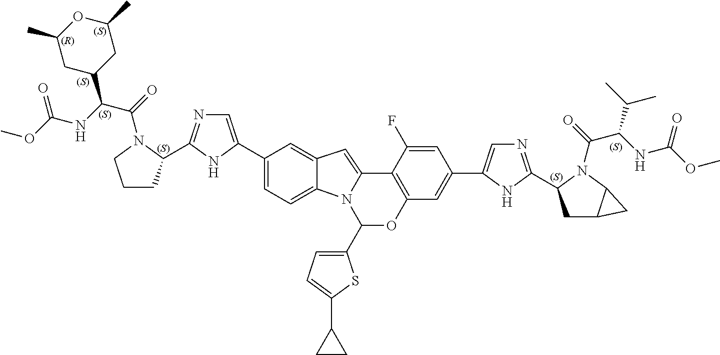 | Isomer 1 Isomer 2 Isomer 3 Isomer 4 Isomer 5 | 1029.48 1029.28 1031.46 1030.39 1029.58 |
| 560 | 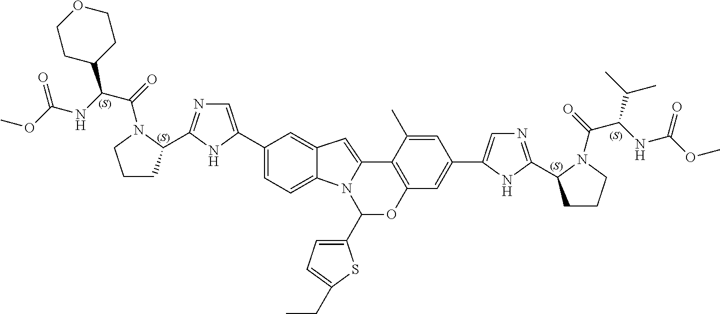 | Isomer 2 | 973.33 |
| 561 562 | 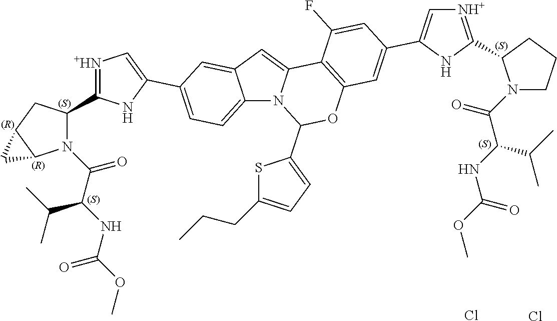 | Isomer 2 Isomer 1 | |
| 563 564 | 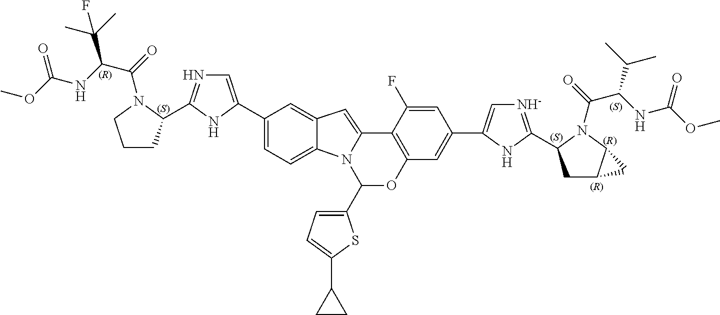 | Isomer 2 Isomer 1 | |
| 565 | 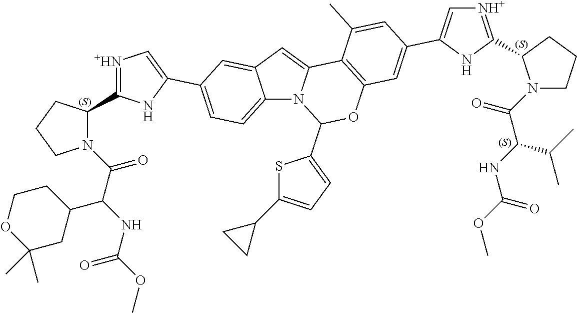 | Isomer 1 | |
| 566 567 | 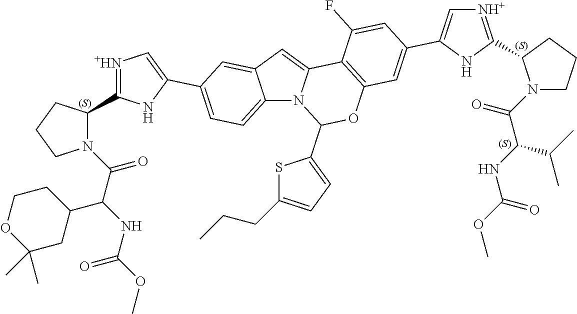 | Isomer 1 Isomer 2 | |
| 568 569 | 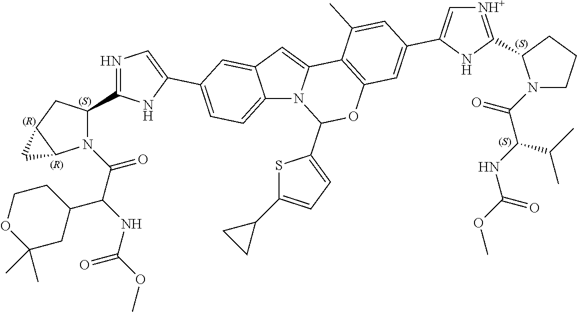 | Isomer 1 Isomer 2 | |
| 570 | 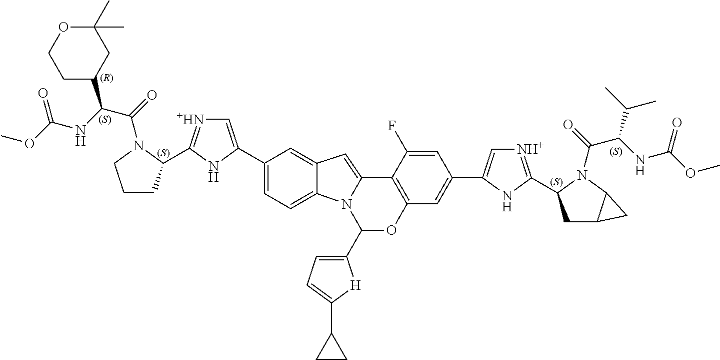 | Isomer 4 | |
| 571 572 | 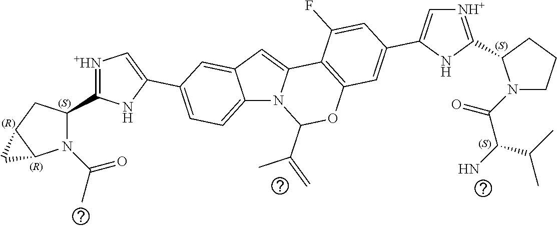 | Isomer 1 Isomer 2 | |
| 614 615 616 | 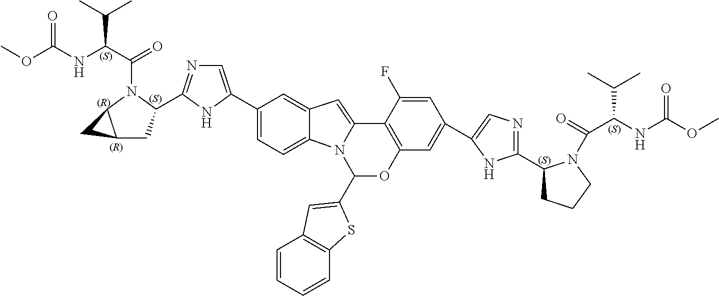 | Isomer 1 racemic Isomer 2 | 969.11 968.77 969.09 |
| 620 621 622 | 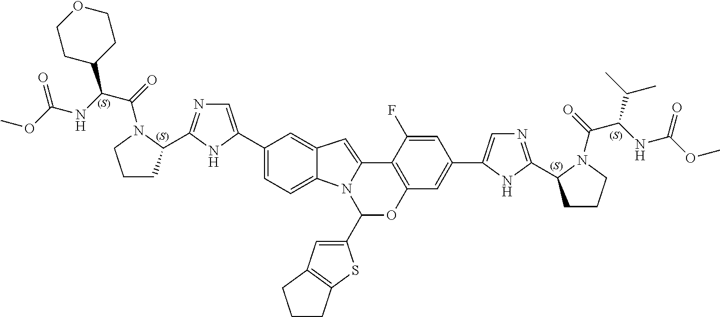 | racemic Isomer 1 Isomer 2 | 989.20 989.16 989.14 |
| 626 627 628 | 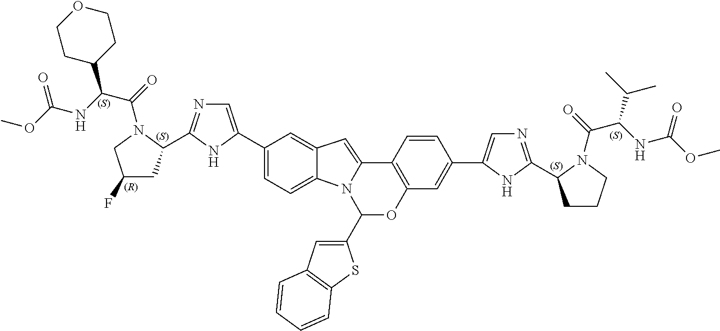 | racemic Isomer 1 Isomer 2 | 998.96 998.95 998.94 |
| 629 630 631 | 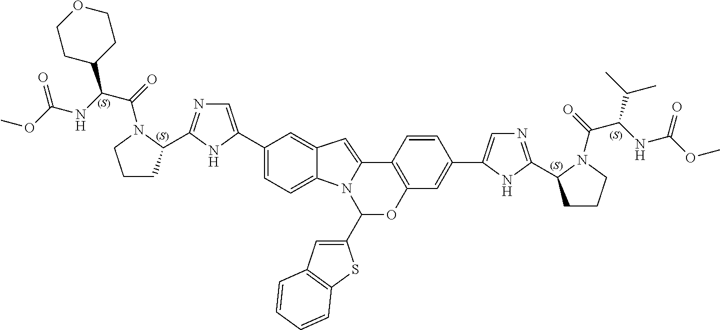 | racemic Isomer 1 Isomer 2 | 998.96 998.95 998.94 |
| 640 641 642 | 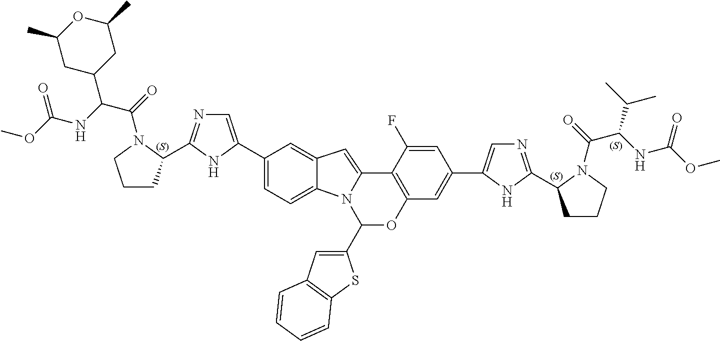 | racemic Isomer 1 Isomer 2 | 1027.20 10.27.38 1027.54 |
| 646 647 648 | 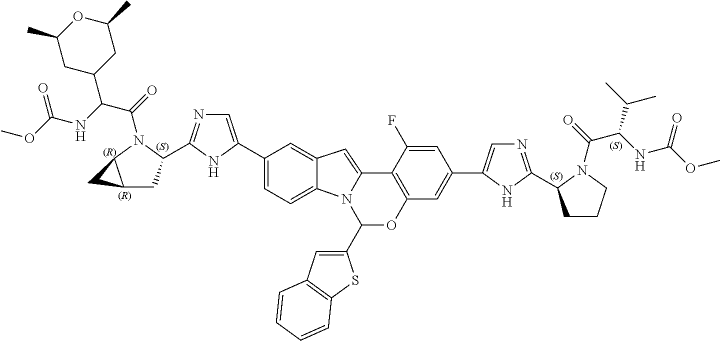 | racemic Isomer 1 Isomer 2 | 1039.21 1040.02 1040.18 |
| 659 660 | 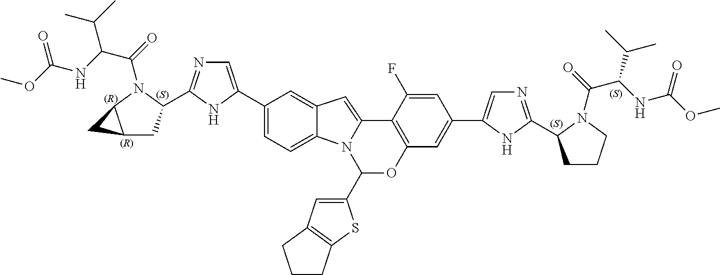 | Isomer 2 Isomer 1 | 959.10 959.10 |
| 706 707 708 | 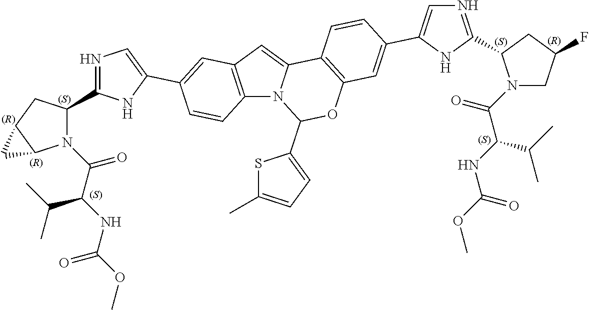 | Isomer 1 Isomer 2 racemic | 932.6 932.4 932.4 |
| 711 | 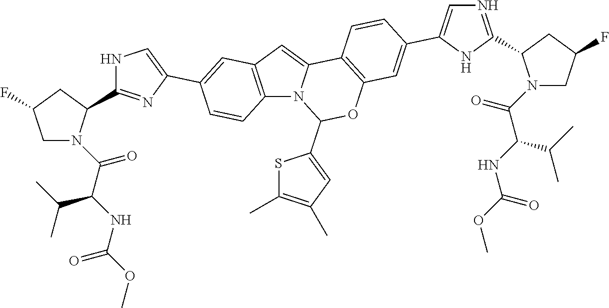 [SMILES: COC(=O)N[C@H](C(=O)N1C[C@H](F)C[C@H]1c1nc(-c2ccc3c(c2)cc2n3C(c3cc(C)c(C)s3)Oc3cc(-c4cnc([C@@H]5C[C@@H](F)CN5C(=O)[C@@H](NC(=O)OC)C(C)C)[nH]4)ccc3-2)c[nH]1)C(C)C] | racemic | 952.7 |
| 716 717 718 | 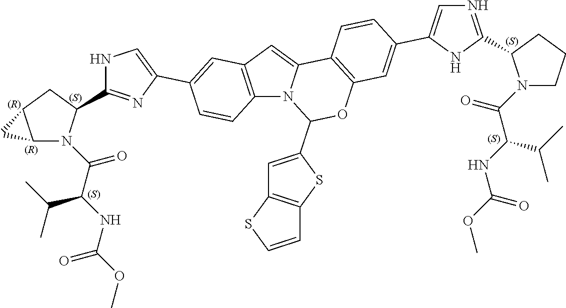 | Isomer 1 racemic Isomer 2 | 956.69 956.69 956.78 |
| 724 | 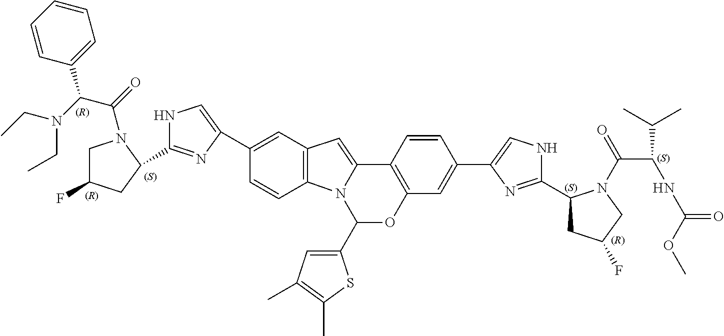 | racemic | 984.4 |
| 725 | 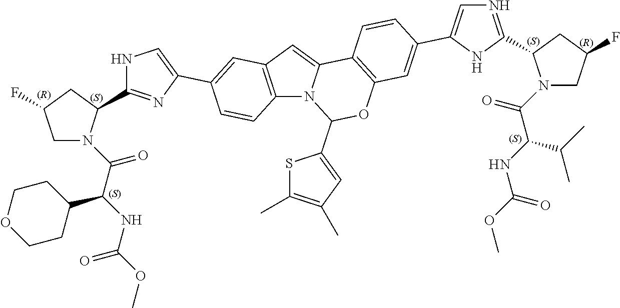 | racemic | 994.0 |
| 726 | 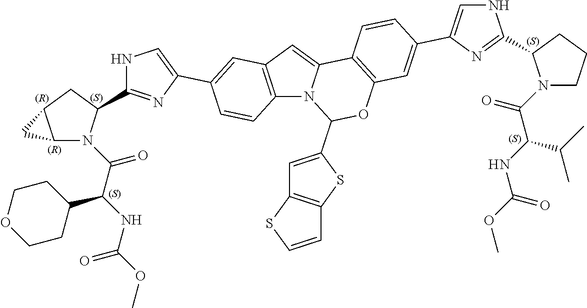 | racemic | 998.92 |
| 729 730 731 | 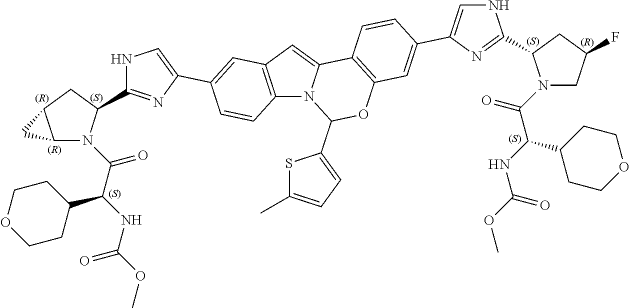 | Isomer 1 racemic Isomer 2 | 1016.0 1016.3 1016.4 |
| 800 801 | 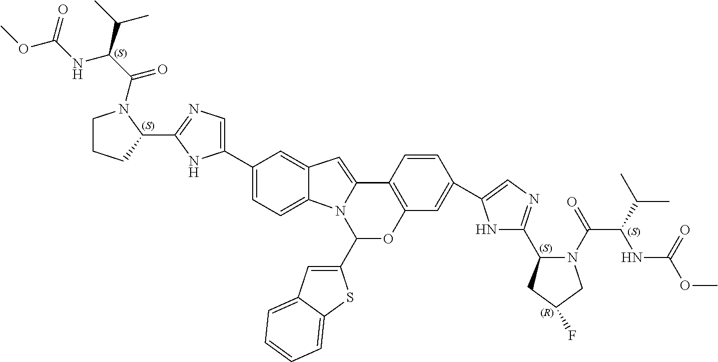 | isomer 1 isomer 2 | 957.80 957.80 |
| 807 | 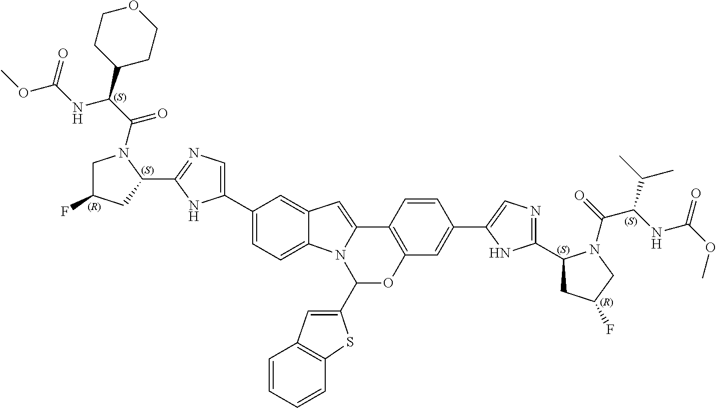 | racemic | 1017.3 |
| 808 | 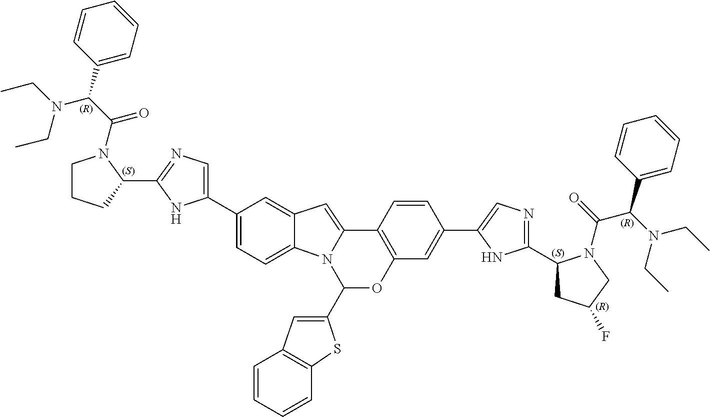 | racemic | 1021.40 |
| 812 813 | 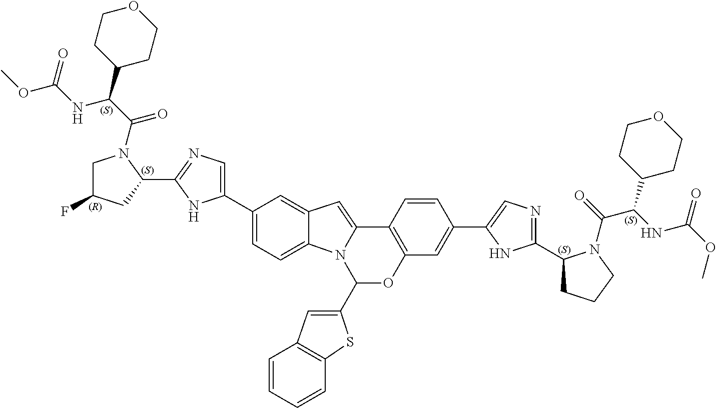 | isomer 1 isomer 2 | 1041.40 1041.40 |

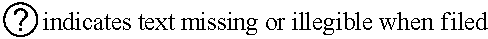

### Example 61

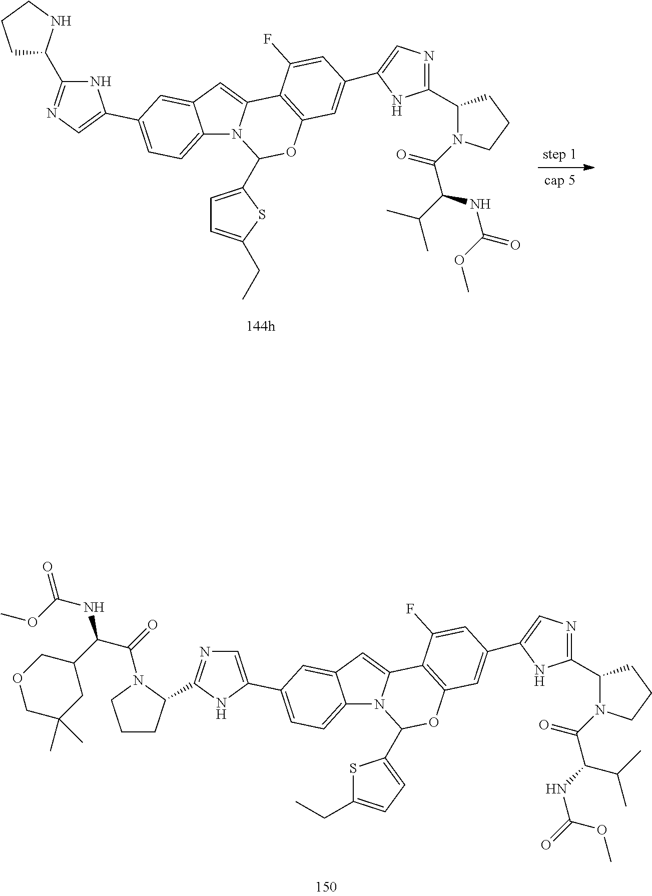 [SMILES: CCc1ccc(C2Oc3cc(-c4cnc([C@@H]5CCCN5C(=O)[C@@H](NC(=O)OC)C(C)C)[nH]4)cc(F)c3-c3cc4cc(-c5cnc([C@@H]6CCCN6)[nH]5)ccc4n32)s1 ; CCc1ccc(C2Oc3cc(-c4cnc([C@@H]5CCCN5C(=O)[C@@H](NC(=O)OC)C(C)C)[nH]4)cc(F)c3-c3cc4cc(-c5cnc([C@@H]6CCCN6C(=O)[C@H](NC(=O)OC)C6COCC(C)(C)C6)[nH]5)ccc4n32)s1]

Compound 144h was prepared in Example 60. To a mixture of 144h (70 mg, 0.09 mmol), cap 5 (22 mg, 0.09 mmol) and DIPEA (0.5 mL) in DMF (5 mL) was added HATU (35 mg, 0.09 mmol). The resulting mixture was allowed to stir at room temperature for 2 hours. The solution was purified by Pre-HPLC to provide 150 (40 mg, 44.4%). ¹H NMR (MeOD) δ: 8.02 (s, 1H), 7.88 (s, 1H), 7.82 (s, 1H), 7.74-7.79 (m, 1H), 7.46-7.58 (m, 2H), 7.33-7.39 (m, 2H), 7.27-7.33 (m, 1H), 7.11-7.23 (m, 1H), 6.51-6.59 (m, 1H), 6.44-6.50 (m, 1H), 5.14-5.27 (m, 2H), 4.15-4.23 (m, 2H), 4.02-4.14 (m, 2H), 3.79-3.90 (m, 2H), 3.64 (s, 8H), 2.62-2.74 (m, 2H), 2.44-2.61 (m, 2H), 1.95-2.31 (m, 8H), 1.50-1.63 (m, 1H), 1.01-1.26 (m, 12H), 0.81-0.98 (m, 6H). LC/MS: Anal. Calcd. For [M+H]⁺ C53H62FN9O8S: 1004.4. found 1004.5.

### Example 62

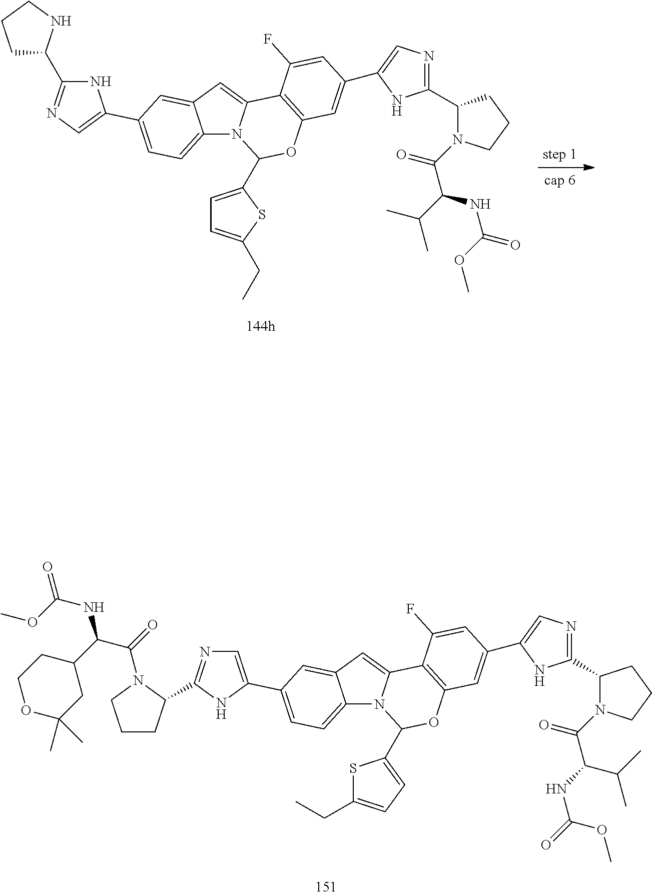 [SMILES: CCc1ccc(C2Oc3cc(-c4cnc([C@@H]5CCCN5C(=O)[C@@H](NC(=O)OC)C(C)C)[nH]4)cc(F)c3-c3cc4cc(-c5cnc([C@@H]6CCCN6)[nH]5)ccc4n32)s1 ; CCc1ccc(C2Oc3cc(-c4cnc([C@@H]5CCCN5C(=O)[C@@H](NC(=O)OC)C(C)C)[nH]4)cc(F)c3-c3cc4cc(-c5cnc([C@@H]6CCCN6C(=O)[C@H](NC(=O)OC)C6CCOC(C)(C)C6)[nH]5)ccc4n32)s1]

Compound 144h was prepared in Example 60. To a mixture of 144h (70 mg, 0.09 mmol), cap 6 (22 mg, 0.09 mmol) and DIPEA (0.5 mL) in DMF (5 mL) was added HATU (35 mg, 0.09 mmol). The resulting mixture was allowed to stir at room temperature for 2 hours. The solution was purified using Pre-HPLC to provide 151 (50 mg, 55.5%). ¹H NMR (MeOD) δ: 8.00 (s, 1H), 7.80-7.89 (m, 2H), 7.72-7.78 (m, 1H), 7.46-7.56 (m, 2H), 7.33-7.41 (m, 1H), 7.25-7.33 (m, 1H), 7.13-7.22 (m, 1H), 6.52-6.59 (m, 1H), 6.46-6.52 (m, 1H), 5.14-5.27 (m, 2H), 4.12-4.23 (m, 3H), 4.02-4.10 (m, 1H), 3.77-3.91 (m, 2H), 3.52-3.72 (m, 8H), 2.62-2.74 (m, 2H), 2.44-2.61 (m, 2H), 1.95-2.32 (m, 8H), 1.56-1.68 (m, 1H), 1.15 (m, 12H), 0.83-0.99 (m, 6H). LC/MS: Anal. Calcd. For [M+H]⁺ C53H62FN9O8S: 100.4. found 1004.5.

### Example 63

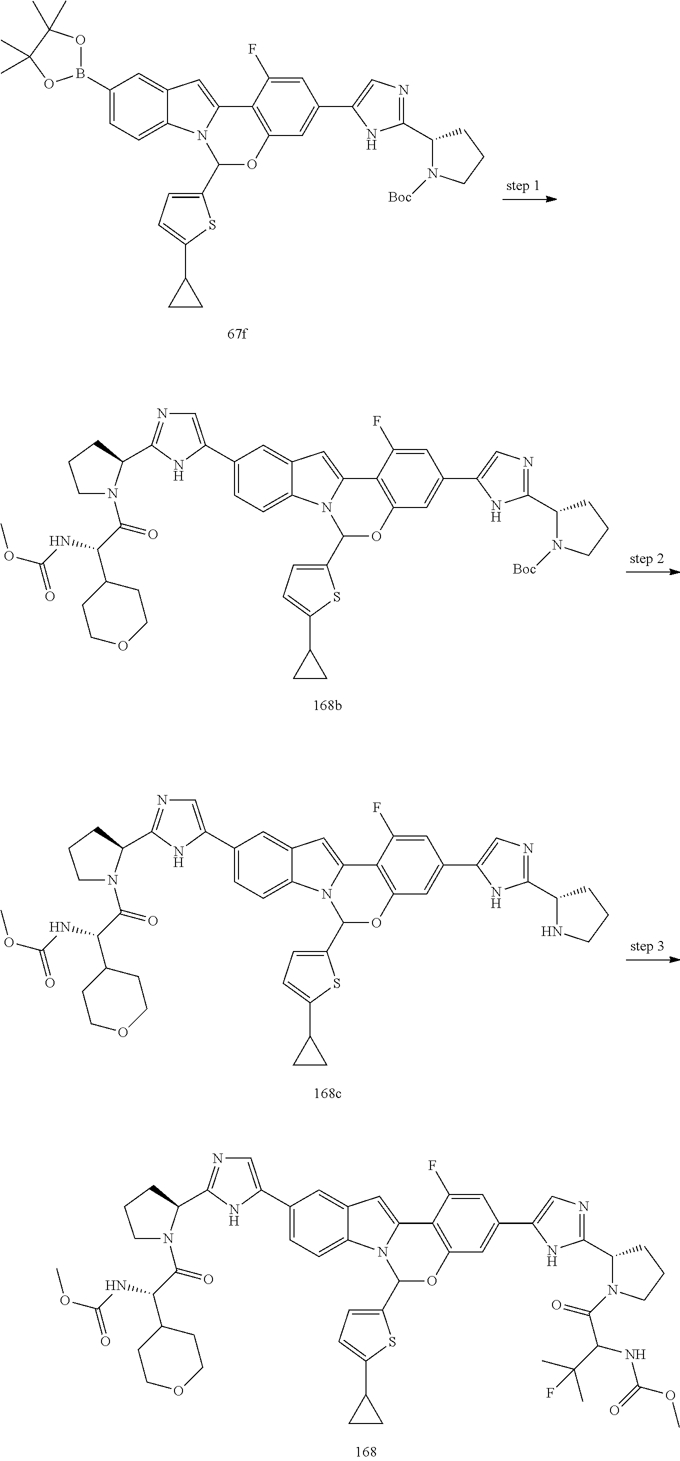

### Step 1

Compound 67f was made in Example 49. A solution of 67f (1.1 g, 1.5 mmol), Pd(dppf)Cl₂ (56 mg, 0.077 mmol), cap 32 (0.76 g, 1.8 mmol), and Na₂CO₃ (484 mg, 4.6 mmol) in (THF/H₂O=5:1, 48 mL) was allowed to stir at 80-100° C. for 16 hours under N₂. The solution was extracted with EtOAc, and the compound organic was dried over Na₂SO₄, concentrated in vacuo, purified using Flash column chromatography on silica gel (20 g, hexane/EtOAc 50%) to provide 168b (0.8 g, 56% yield). LC/MS: Anal. Calcd. For [M+H]⁺ C50H55FN8O7S: 933.0. found 932.9.

### Step 2

To a solution of 168b (800 mg, 0.86 mmol) in dioxane (20 mL) was added HCl/dioxane (15 mL, 4 M) dropwise at 20° C. The solution was allowed to stir at 20° C. for 6 hours and concentrated in vacuo to provide 168c (714 mg, 100%). LC/MS: Anal. Calcd. For [M+H]⁺ C45H47FN8O5S; 831.97. found 832.

### Step 3

To a solution of 168c (714 mg, 0.86 mmol), DIPEA (222 mg, 1.7 mmol), cap 1 (153 mg, 0.86 mmol), in 10 mL DMF was added HATU (327 mg, 0.86 mmol) and stirred at 20° C. for 2 hours. The solution was purified using Pre-HPLC to provide 168 (301 mg, 36%). ¹H NMR (MeOD) δ: 8.00-8.03 (m, 1H), 7.95 (s, 1H), 7.82 (s, 1H), 7.75 (s, 1H), 7.51 (q, J=8.6 Hz, 2H), 7.39 (d, J=11.3 Hz, 1H), 7.32 (s, 1H), 7.20 (br. s., 1H), 6.44-6.51 (m, 2H), 5.22 (q, J=7.3 Hz, 2H), 4.70 (d, J=9.0 Hz, 1H), 4.26 (d, J=8.2 Hz, 1H), 4.11 (br. s., 1H), 3.85-4.03 (m, 5H), 3.65 (d, J=6.7 Hz, 6H), 3.33 (s, 2H), 2.55 (br. s., 2H), 2.13-2.31 (m, 5H), 1.88-2.01 (m, 2H), 1.59 (d, J=12.9 Hz, 1H), 1.21-1.46 (m, 11H), 0.87-0.91 (m, 2H), 0.53 (d, J=3.5 Hz, 2H). LC/MS: Anal. Calcd. For [M+H]⁺ C52H57F2N9O8S; 1008.1. found 1008.4.

### Example 64

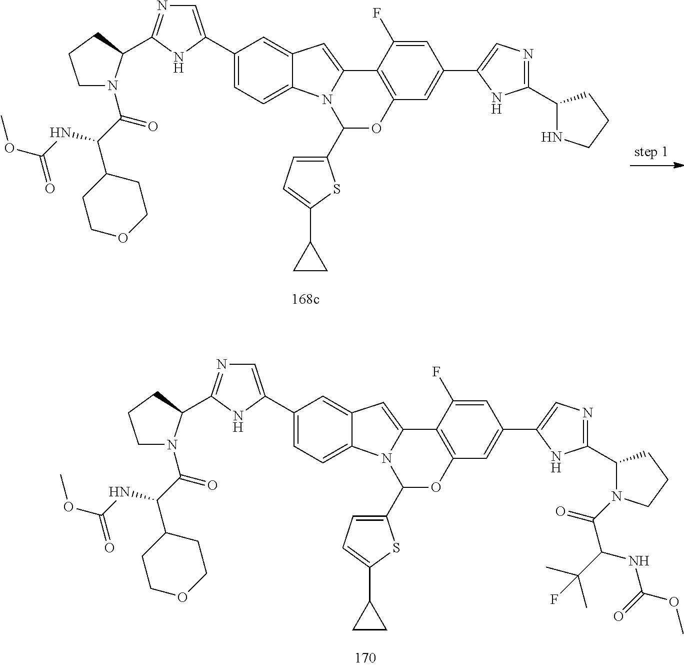

Compound 168c was made in Example 63. To a solution of 168c (446 mg, 0.54 mmol), DIPEA (138 mg, 1.07 mmol) and isomer of cap 1 (95.67 mg, 0.54 mmol) in DMF (8 mL) was added HATU (204 mg, 0.54 mmol). The mixture was allowed to stir at 20° C. for 2 hours. The solution was purified using Pre-HPLC to provide 170 (190 mg, 35%). ¹H NMR (MeOD) δ: 8.01 (s, 1H), 7.89 (s, 1H), 7.80 (s, 1H), 7.75 (s, 1H), 7.46-7.54 (m, 2H), 7.30-7.39 (m, 2H), 7.18 (br. s., 1H), 6.47 (m, J=3.1 Hz, 2H), 5.17-5.25 (m, 2H), 4.70 (d, J=9.4 Hz, 1H), 4.26 (d, J=8.2 Hz, 1H), 4.12 (br. s., 1H), 3.85-4.01 (m, 5H), 3.65 (d, J=6.7 Hz, 6H), 3.36 (d, J=12.5 Hz, 2H), 2.54 (d, J=6.3 Hz, 2H), 2.11-2.28 (m, 5H), 1.93 (d, J=8.2 Hz, 2H), 1.60 (d, J=12.9 Hz, 1H), 1.22-1.45 (m, 11H), 0.89 (d, J=7.8 Hz, 2H), 0.54 (d, J=3.1 Hz, 2H). LC/MS: Anal. Calcd. For [M+H]⁺ C52H57F2N9O8S; 1008.1. found 1008.2.

### Example 65

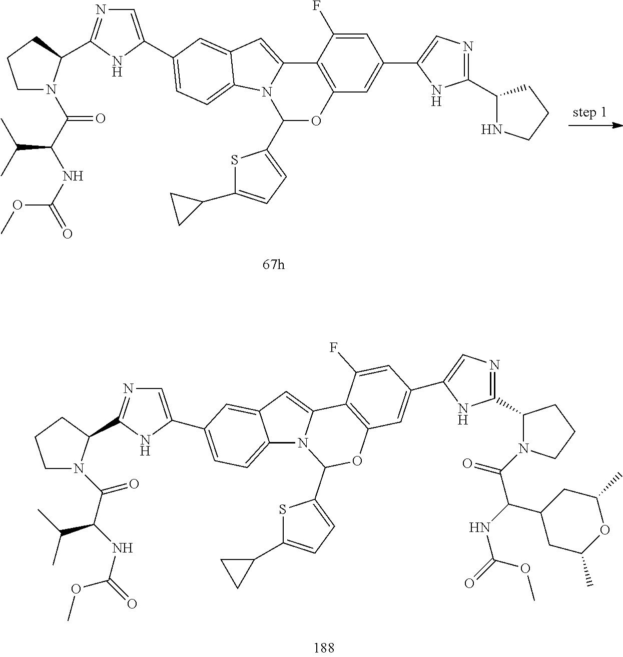 [SMILES: COC(=O)N[C@H](C(=O)N1CCC[C@H]1c1ncc(-c2ccc3c(c2)cc2n3C(c3ccc(C4CC4)s3)Oc3cc(-c4cnc([C@@H]5CCCN5)[nH]4)cc(F)c3-2)[nH]1)C(C)C ; COC(=O)NC(C(=O)N1CCC[C@H]1c1ncc(-c2cc(F)c3c(c2)OC(c2ccc(C4CC4)s2)n2c-3cc3cc(-c4cnc([C@@H]5CCCN5C(=O)[C@@H](NC(=O)OC)C(C)C)[nH]4)ccc32)[nH]1)C1C[C@@H](C)O[C@@H](C)C1]

Compound 67h was made in Example 49. To a mixture of 67h (500 mg, 0.63 mmol), cap 4 (186 mg, 0.76 mmol) and HATU (289 mg, 0.76 mmol) in DMF (5 mL) was added DIEA (164 mg, 1.26 mmol). The resulting mixture was allowed to stir at 20° C. for 30 minutes, and LC-MS judged the material was consumed up. After filtrated, the filtrate was purified using Pre-HPLC to provide 188 (290 mg, 45% yield). ¹H NMR (MeOD) δ: 7.95-8.00 (m, 1H), 7.86 (s, 1H), 7.71-7.78 (m, 2H), 7.43-7.54 (m, 2H), 7.37 (d, J=10.4 Hz, 1H), 7.24-7.30 (m, 1H), 7.15 (s, 1H), 6.49 (s, 2H), 5.14-5.27 (m, 2H), 4.19-4.27 (m, 2H), 4.08 (m, 2H), 3.85 (d, J=6.8 Hz, 2H), 3.64 (s, 6H), 3.34-3.46 (m, 3H), 2.45-2.60 (m, 2H), 1.97-2.30 (m, 8H), 1.89-1.95 (m, 1H), 1.54 (d, J=12.4 Hz, 1H), 1.30 (d, J=12.0 Hz, 1H), 1.11-1.18 (m, 1H), 1.07 (d, J=4.4 Hz, 6H), 0.84-0.99 (m, 9H), 0.53 (d, J=3.6 Hz, 2H). LC/MS: Anal. Calcd. For [M+H]⁺ C54H62FN9O8S; 1017.45. found 1017.3.

### Example 66

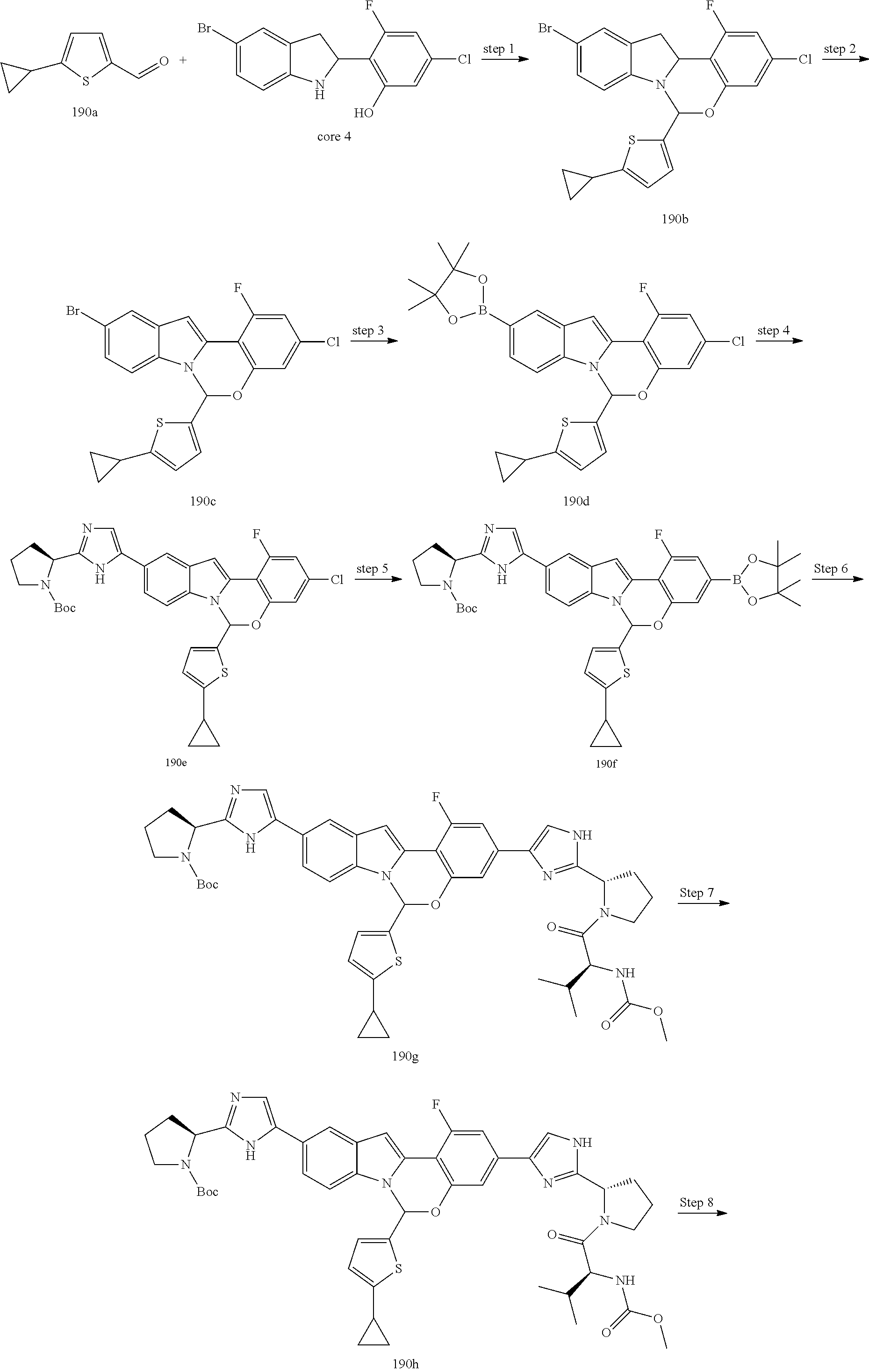

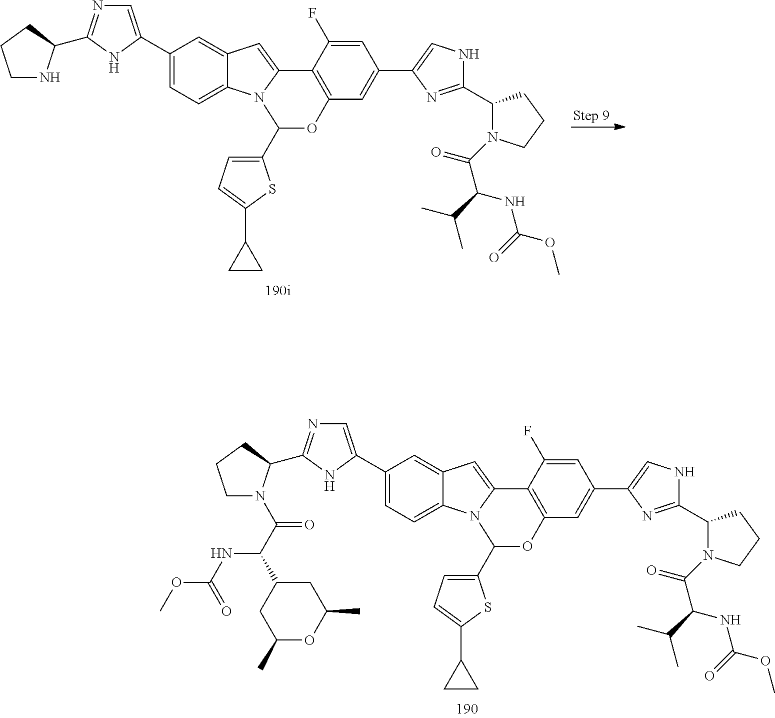

### Step 1

To a mixture of 190a (2.7 g, 17.5 mmol) and core 4 (4 g, 11.7 mmol) in anhydrous CH₃CN (50 mL) was added TFA (1 mL). The mixture was allowed to stir at 20° C. for 6 hours. The reaction mixture became a clear solution and then a solid appeared. The solid was collected by filtration and washed with CH₃CN to provide 190b (4.5 g, 81% yield). ¹H NMR (CDCl₃) δ: 7.21 (s, 2H), 6.52-6.78 (m, 6H), 5.07 (d, J=9.6 Hz, 1H), 3.50 (dd, J=16.4, 9.2 Hz, 1H), 3.19 (d, J=16.4 Hz, 1H), 1.91-1.96 (m, 1H), 0.88-0.93 (m, 2H), 0.63-0.65 (m, 2H).

### Step 2

The solution of 190b (4.5 g, 9.4 mmol) in dry toluene (50 mL) was added DDQ (3.2 g, 14.2 mmol). After refluxing for 2 hours, the solvent was removed in vacuo and diluted with EtOAc. The organic layer was washed with saturated Na₂SO₃ aqueous and brine, dried over Na₂SO₄, filtered and concentrated in vacuo. The residue obtained was washed with MeOH (20 mL). The solid was collected to provide 190c (4 g, 88% yield).

### Step 3

A suspension of 190c (4.0 g, 8.4 mmol), bis(pinacolato)diboron (2.6 g, 10.1 mmol), KOAc (2.1 g, 21.1 mmol) and Pd(dppf)Cl₂ (310 mg, 0.42 mmol) in dioxane (50 mL) was allowed to stir at 100° C. for 2 hours under N₂ atmosphere. The reaction mixture was cooled and concentrated in vacuo, and the residue obtained was purified using Flash column chromatography on silica gel (80 g, EtOAc/Hexane 0% to 5%) to provide 190d (4 g, 88% yield).

### Step 5

A suspension of 190d (4.4 g, 8.4 mmol), cap 31a (3.2 g, 8.4 mmol), Na₂CO₃ (2.2 g, 21.0 mmol) and Pd(dppf)Cl₂ (310 mg, 0.42 mmol) in THF/H₂O (v/v=5/1, 120 mL) was allowed to stir at 80° C. for about 15 hours under N₂ atmosphere. After that, the mixture was washed with water and extracted with ethyl acetate, washed with brine and dried over anhydrous sodium sulfate. After being concentrated in vacuo, the residue obtained was purified using Flash column chromatography on silica gel (80 g, EtOAc/Hexane 10% to 50%) to provide 190e (4.5 g, 77% yield). LC/MS: Anal. Calcd. For [M+H]⁺ C36H35ClFN5O4S; 688.21. found 688.3.

### Step 5

To a mixture of 190e (4.5 g, 6.5 mmol), bis(pinacolato)diboron (2.0 g, 7.8 mmol), KOAc (1.6 g, 16.3 mmol), Pd₂(dba)₃ (338 mg, 0.33 mmol), X-Phos (312 mg 0.65 mmol) degassed and sealed under N₂ was added dry dioxane. Following further N₂ purging. The mixture was allowed to stir at 100° C. for about 15 hours. After cooling to 20° C., the solvent was concentrated in vacuo and the residue obtained was purified using Flash column chromatography on silica gel (3 g, Hexane/EtOAc 20% to 50%) to provide 190f (4.6 g, 89% yield). LC/MS: Anal. Calcd. For [M+H]⁺ C42H47BFN5O6S; 780.33. found 780.4.

### Step 6

A suspension of 190f (4.2 g, 5.8 mmol), cap 31 (3.25 g, 8.7 mmol), Na₂CO₃ (1.5 g, 14.5 mmol) and Pd(dppf)Cl₂ (222 mg, 0.29 mmol) in THF/H₂O (72 mL, 5:1) was allowed to stir at 100° C. under N₂ atmosphere for about 15 hours. After filtration, the filtrate was washed with water (50 mL) and extracted with ethyl acetate (100 mL). The organic layer was washed with brine and dried over Na₂SO₄. After filtration and concentration, the residue obtained was purified using Flash column chromatography on silica gel (2 g, MeOH/DCM 0% to 20%) to provide 190g (3.8 g, 74% yield). LC/MS: Anal. Calcd. For [M+H]⁺ C48H53FN8O6S; 889.38. found 889.4.

### Step 7

The compound 190g (3.8 g) was separated by SFC by using the following conditions to provide 190h (1 g, 52.6% yield).

Column: Chiralpak AS-H 250×4.6 mm I.D., Sum

Mobile phase: 40% of iso-propanol (0.05% DEA) in CO₂

Flow rate: 60 mL/min

Wavelength: 220 nm

### Step 8

To a solution of compound 190h (1 g, 1.1 mmol) in dry dioxane (10 mL) was added HCl-dioxane (20 mL) through syringe and stirred at 20° C. for 2 hours, then concentrated in vacuo and dried under high vacuum to provide HCl salt of 190i (0.9 g, 98% yield). LC/MS: Anal. Calcd. For [M+H]⁺ C43H45FN8O4S; 789.33. found 789.5.

### Step 9

To a mixture of 190i (150 mg, 0.19 mmol), cap 4 (46.6 mg, 0.19 mmol) and DIPEA (73.5 mg, 0.57 mmol) in DMF (5 mL) was added HATU (72.2 mg, 0.19 mmol). The resulting mixture was allowed to stir at 20° C. LCMS judged the material was consumed up. It was filtered, and the filtrate was purified using Pre-HPLC to provide the 190 (50 mg, 29%). ¹H NMR (MeOD) δ: 8.02 (s, 1H), 7.91 (s, 1H), 7.80 (m, 2H), 7.55-7.45 (m, 2H), 7.40-7.35 (m, 1H), 7.30 (s, 1H), 7.20 (s, 1H), 6.61-6.52 (m, 2H), 5.25-5.15 (m, 2H), 4.25-4.10 (m, 4H), 4.10-4.05 (m, 1H), 3.89-3.76 (m, 2H), 3.70 (m, 1H), 3.63 (m, 6H), 2.60-2.49 (m, 2H), 2.31-2.20 (m, 2H), 2.20-2.10 (m, 4H), 2.05-2.00 (m, 1H), 1.95 (m, 1H), 1.60 (m, 1H), 1.55 (m, 1H), 1.25 (m, 1H), 1.20 (m, 3H), 1.10 (m, 1H), 1.02-0.98 (m, 3H), 0.95 (m, 1H), 0.90-0.85 (m, 8H), 0.52 (s, 2H). LC/MS: Anal. Calcd. For [M+H]⁺ C54H62FN9O8S; 1016.44. found 1016.54.

### Example 67

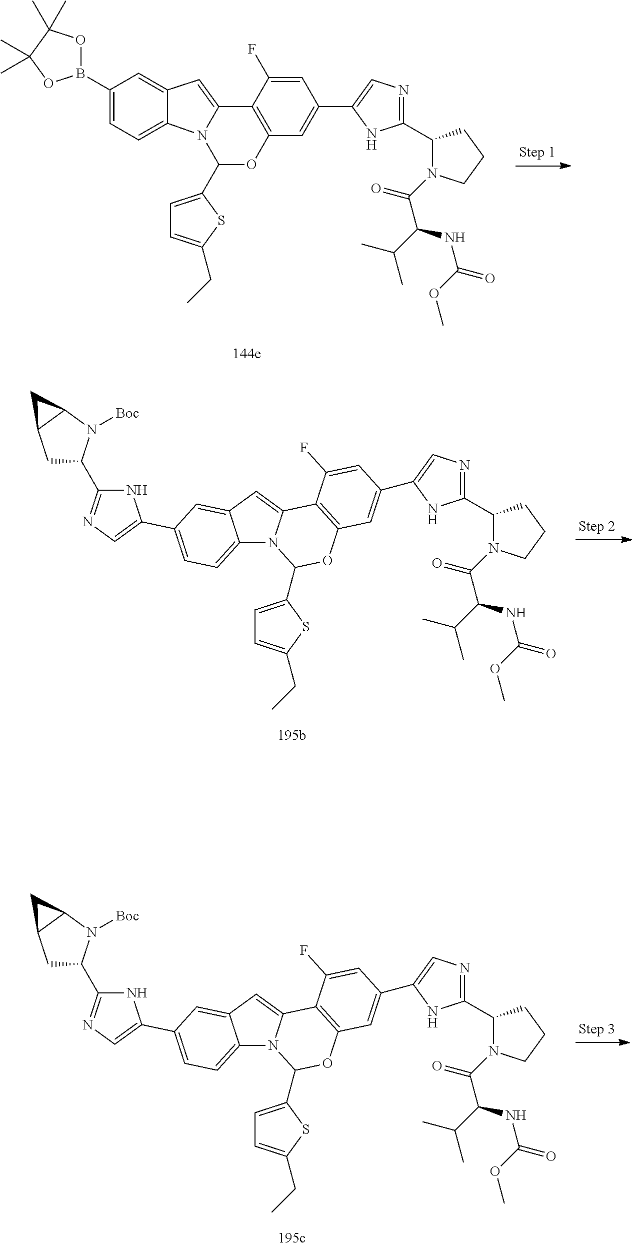 [SMILES: CCc1ccc(C2Oc3cc(-c4cnc([C@@H]5CCCN5C(=O)[C@@H](NC(=O)OC)C(C)C)[nH]4)cc(F)c3-c3cc4cc(B5OC(C)(C)C(C)(C)O5)ccc4n32)s1 ; CCc1ccc(C2Oc3cc(-c4cnc([C@@H]5CCCN5C(=O)[C@@H](NC(=O)OC)C(C)C)[nH]4)cc(F)c3-c3cc4cc(-c5cnc([C@@H]6C[C@H]7C[C@H]7N6C(=O)OC(C)(C)C)[nH]5)ccc4n32)s1 ; CCc1ccc(C2Oc3cc(-c4cnc([C@@H]5CCCN5C(=O)[C@@H](NC(=O)OC)C(C)C)[nH]4)cc(F)c3-c3cc4cc(-c5cnc([C@@H]6C[C@H]7C[C@H]7N6C(=O)OC(C)(C)C)[nH]5)ccc4n32)s1]

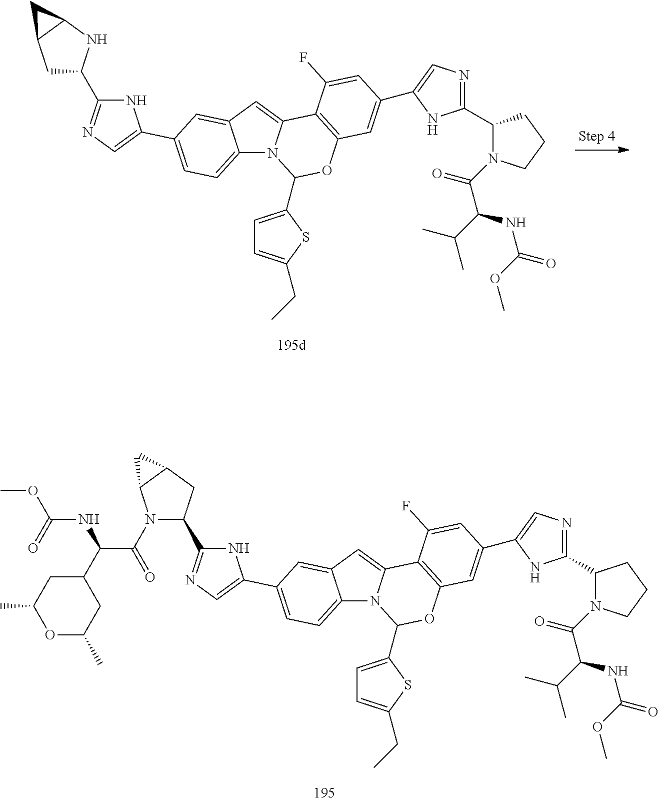

### Step 1

Compound 144e was prepared in Example 60. A suspension of 144e (2 g, 2.6 mmol), cap 31a (0.8 g, 2.6 mmol), Na₂CO₃ (800 mg, 7.8 mmol) and Pd(dppf)Cl₂ (190 mg, 0.26 mmol) in THF/H₂O (50 mL) was allowed to stir at 90° C. under N₂ atmosphere for about 15 hours. The reaction mixture was concentrated in vacuo and purified using Flash column chromatography on silica gel (30 g, MeOH/DCM 0% to 3%) to provide 195b (1.4 g, 61.4% yield). LC/MS: Anal. Calcd. For [M+H]⁺ C48H53FN8O6S; 889.38. found 889.5.

### Step 2

The two diastereo isomers of 195b (1.4 g) were separated by SFC by using the following condition.

Column: OZ-H

Solvent: EtOH(0.05% DEA)

Flow rate: 2 mL/min

Wavelength: 220 nm

195c_1 (500 mg, 71.4%). LC/MS: Anal. Calcd. For [M+H]⁺ C48H53FN8O6S: 889.38. found 889.6.

195c_2 (510 mg, 72.8%). LC/MS: Anal. Calcd. For [M+H]⁺ C48H53FN8O6S: 889.38. found 889.6.

### Step 3

To a solution of 195c (0.5 g, 0.56 mmol) in dioxane (2 mL) was added HCl/dioxane (0.5 mL, 4 M) through syringe and stirred at 20° C. for 2 hours, then concentrated in vacuo and dried under high vacuum to provide 195d (443 mg, 100%). LC/MS: Anal. Calcd. For [M+H]⁺ C43H45FN8O4S; 789.33. found 789.5.

### Step 4

To a mixture of 195d (150 mg, 0.19 mmol), cap 4 (47 mg, 0.19 mmol) and DIPEA (0.5 mL) in DMF (5 mL) was added HATU (74 mg, 0.19 mmol). The resulting mixture was allowed to stir at 20° C. for 2 hours before the solution was subjected directly to Pre-HPLC to provide 195 (50 mg, 39.3%). ¹H NMR (MeOD) δ: 8.01 (s, 1H), 7.84 (d, J=14.1 Hz, 2H), 7.75 (s, 1H), 7.45-7.55 (m, 2H), 7.33-7.40 (m, 1H), 7.30 (s, 1H), 7.17 (d, J=2.7 Hz, 1H), 6.46-6.57 (m, 2H), 5.18 (t, J=7.2 Hz, 1H), 5.09 (t, J=7.8 Hz, 1H), 4.55 (d, J=7.0 Hz, 1H), 4.19 (d, J=7.0 Hz, 1H), 4.06 (br. s., 1H), 3.76-3.87 (m, 2H), 3.64 (d, J=4.7 Hz, 6H), 2.61-2.73 (m, 3H), 2.40-2.54 (m, 2H), 1.95-2.28 (m, 6H), 1.40-1.59 (m, 2H), 1.00-1.18 (m, 11H), 0.80-0.99 (m, 9H). LC/MS: Anal. Calcd. For [M+H]⁺ C54H62FN9O8S; 1016.44. found 1016.5.

The following compounds of the present invention were made using the methods described in the Example above and substituting the appropriate reactants or reagents.

| | | | Observed |
|---|---|---|---|
| ID | Structure | Isomer | [M + H]+ |
| 414 415 | 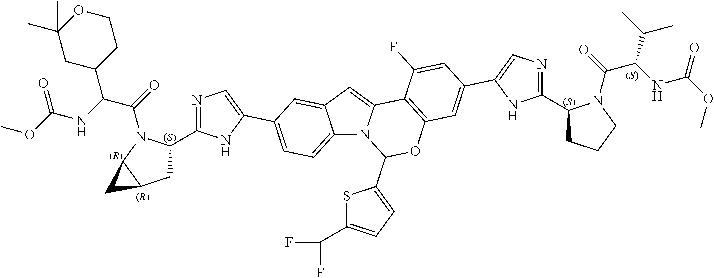 | Isomer 1 Isomer 2 | 1038.1 1038.1 |
| 416 417 418 419 | 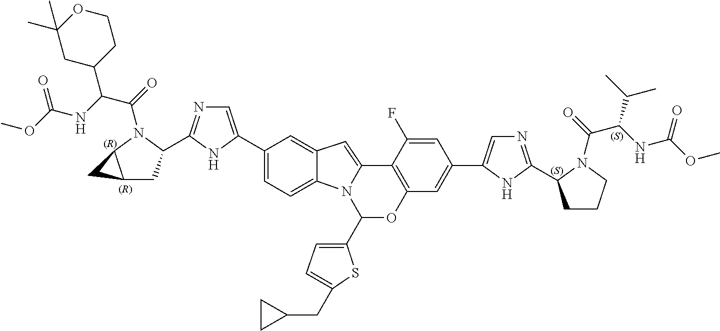 | Isomer 1 Isomer 2 Isomer 3 Isomer 4 | 1042.2 1042.2 1042.2 1042.2 |

### Example 68

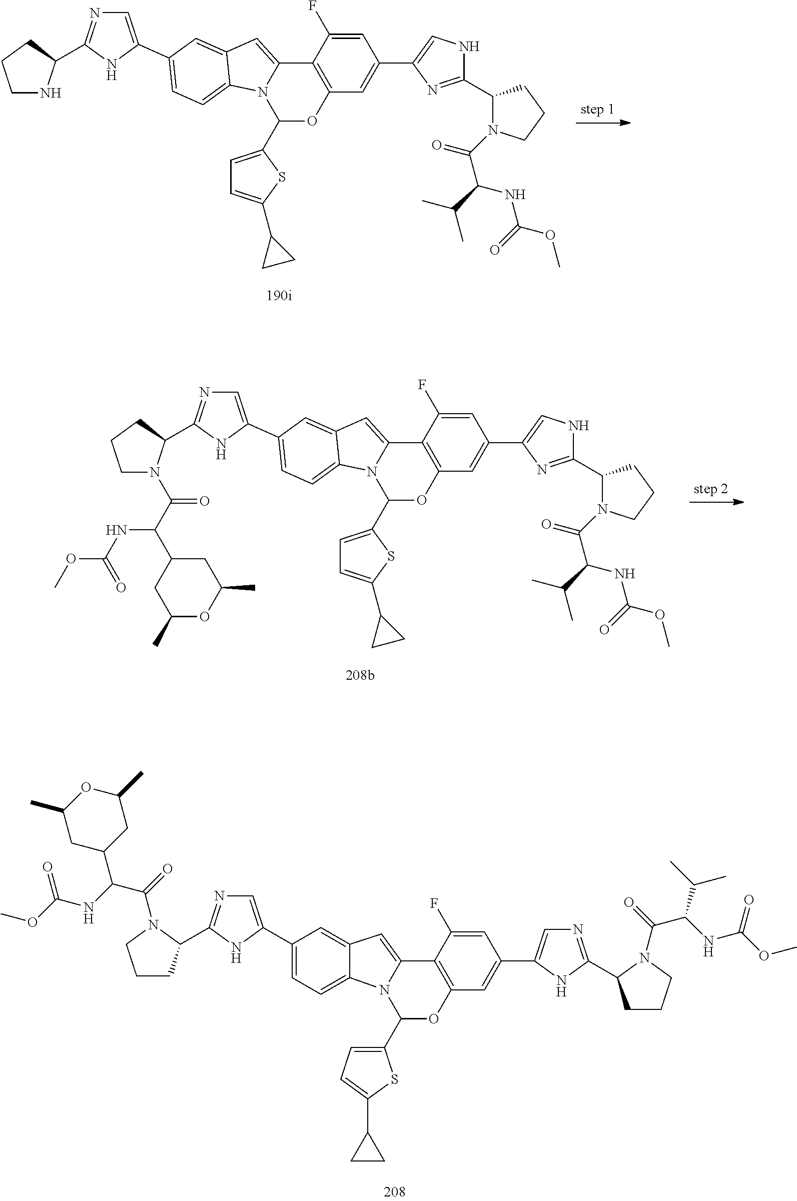

### Step 1

Compound 190i was made in Example 66. To a mixture of 190i (1.6 g, 2.02 mmol), cap 4 (496 mg, 2.02 mmol) and DIPEA (526 mg, 4.04 mmol) in DMF (8 mL) was added HATU (767 mg, 2.02 mmol), the resulting mixture was allowed to stir at 20° C. for 2 hours. The mixture was extracted with EOAc, washed with brine, dried over Na₂SO₄. The organic layer was dried over Na₂SO₄. After filtration and concentration, the residue obtained was purified using Flash column chromatography on silica gel (20 g, DCM/MeOH 0% to 10%) to provide 208b (1.5 g, 73% yield). LC/MS: Anal. Calcd. For [M+H]⁺ C54H62FN9O8S; 1016.44. found 1016.6.

### Step 2

The compound 208b (620 mg, 20% yield) was separated from 208b (1.5 g) by SFC by using the following conditions.

Column: Chiralpak AS-H 250×4.6 mm I.D., Sum

Mobile phase: 40% of iso-propanol (0.05% DEA) in CO₂

Flow rate: 2.5 mL/min

Wavelength: 340 nm

¹H NMR (MeOD) δ: 8.03-8.09 (m, 1H), 7.93 (s, 1H), 7.76 (s, 2H), 7.50-7.60 (m, 1H), 7.35-7.48 (m, 2H), 7.22-7.29 (m, 1H), 7.10-7.17 (m, 1H), 6.39-6.51 (m, 2H), 5.17-5.27 (m, 2H), 4.19-4.31 (m, 2H), 3.87-4.13 (m, 4H), 3.64 (s, 6H), 3.40-3.51 (m, 2H), 2.49-2.61 (m, 2H), 2.01-2.35 (m, 8H), 1.86-1.97 (m, 1H), 1.46-1.57 (m, 1H), 1.29-1.38 (m, 1H), 1.07 (m, 6H), 0.90 (m, 10H), 0.46-0.56 (m, 2H). LC/MS: Anal. Calcd. For [M+H]⁺ C54H62FN9O8S; 1016.44. found 1016.6.

### Example 69

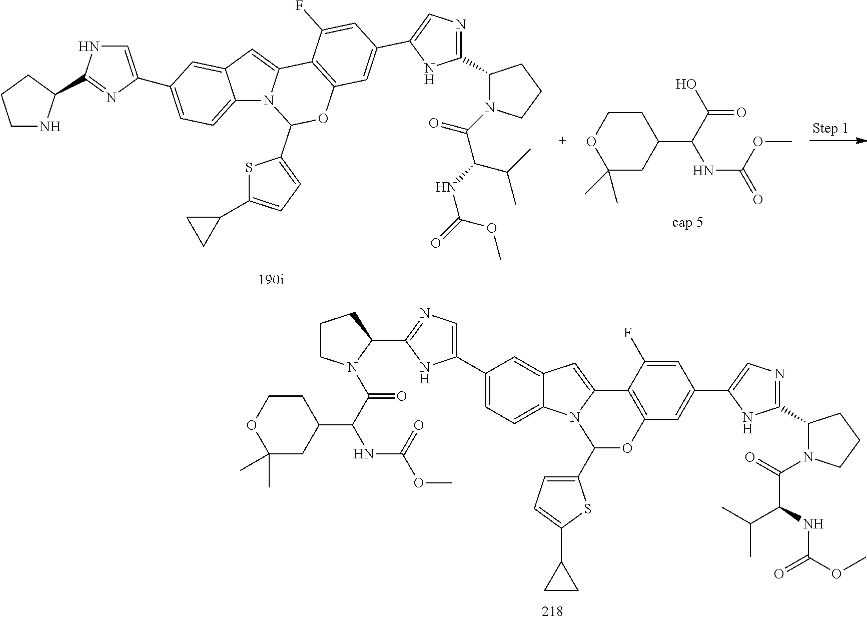

Compound 190i was prepared using the method described in Example 66. To a mixture of compound 190i (120 mg, 0.15 mmol), cap 5 (37 mg, 0.15 mmol) and HATU (58 mg, 0.15 mmol) in DMF (4 mL) was added DIEA (39 mg, 0.30 mmol). The resulting reaction was allowed to stir at 20° C. for about 15 hours, and then the reaction mixture was purified directly using RPLC to provide compound 218 (81 mg, 53% yield). ¹H NMR (MeOD) δ: 8.00-8.07 (m, 1H), 7.90 (s, 1H), 7.73-7.83 (m, 2H), 7.43-7.57 (m, 2H), 7.36 (d, J=10.6 Hz, 1H), 7.29 (s, 1H), 7.17 (m, 1H), 6.41-6.53 (m, 2H), 5.20 (m, 2H), 4.19 (t, J=7.2 Hz, 2H), 4.07 (d, J=8.6 Hz, 2H), 3.78-3.90 (m, 2H), 3.57-3.72 (m, 8H), 2.47-2.60 (m, 2H), 1.97-2.31 (m, 8H), 1.88-1.97 (m, 1H), 1.57 (d, J=12.0 Hz, 1H), 1.19 (d, J=8.2 Hz, 4H), 1.07 (m, 6H), 0.83-0.99 (m, 8H), 0.53 (d, J=3.6 Hz, 2H). LC/MS: Anal. Calcd. For [M+H]⁺ C54H62FN9O8S; 1016.44. found 1016.6.

### Example 70

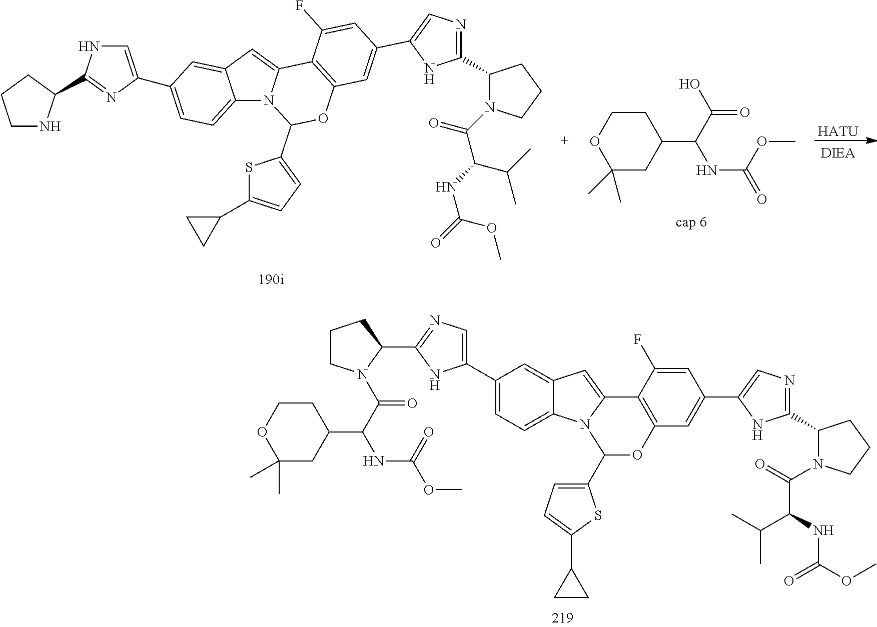

Compound 190i was prepared in Example 66. To a mixture of 190i (120 mg, 0.15 mmol), cap 6 (37 mg, 0.15 mmol) and HATU (58 mg, 0.15 mmol) in DMF (4 mL) was added DIEA (39 mg, 0.30 mmol). The resulting reaction was allowed to stir at 20° C. for about 15 hours, and then the reaction mixture was directly purified using RPLC to provide compound 219 as a mixture of isomers, referred to herein as compounds 220-225 (76 mg, 49% yield). ¹H NMR (MeOD) δ: 8.00 (s, 1H), 7.90 (s, 1H), 7.79 (s, 1H), 7.75 (s, 1H), 7.50 (q, J=8.5 Hz, 2H), 7.36 (d, J=11.0 Hz, 1H), 7.30 (s, 1H), 7.18 (d, J=2.3 Hz, 1H), 6.43-6.52 (m, 2H), 5.15-5.26 (m, 2H), 4.03-4.22 (m, 4H), 3.84 (m, 2H), 3.55-3.70 (m, 8H), 2.54 (d, J=6.7 Hz, 2H), 1.97-2.31 (m, 8H), 1.89-1.96 (m, 1H), 1.62 (m, 1H), 1.08-1.33 (m, 10H), 0.83-0.98 (m, 8H), 0.53 (d, J=4.7 Hz, 2H). LC/MS: Anal. Calcd. For [M+H]⁺ C54H62FN9O8S; 1016.44. found 1016.6.

### Example 71

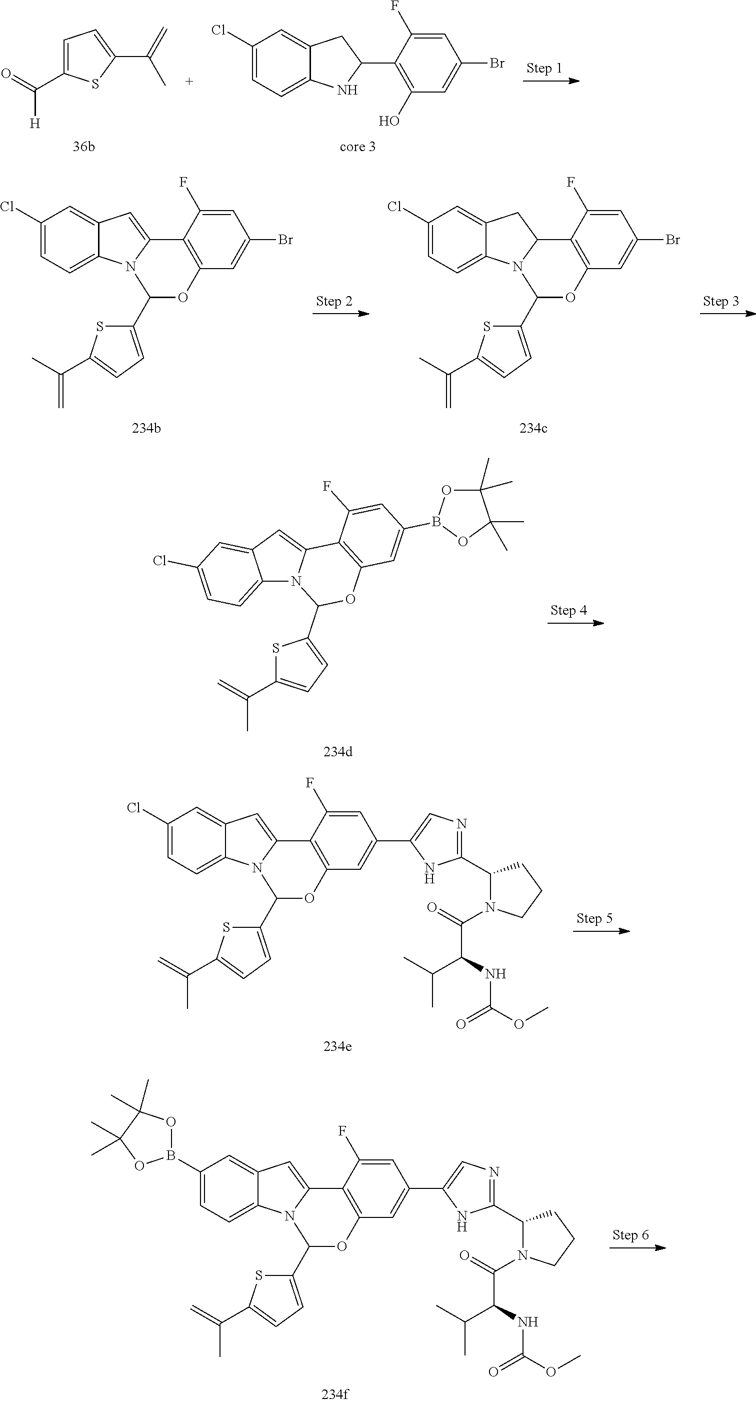

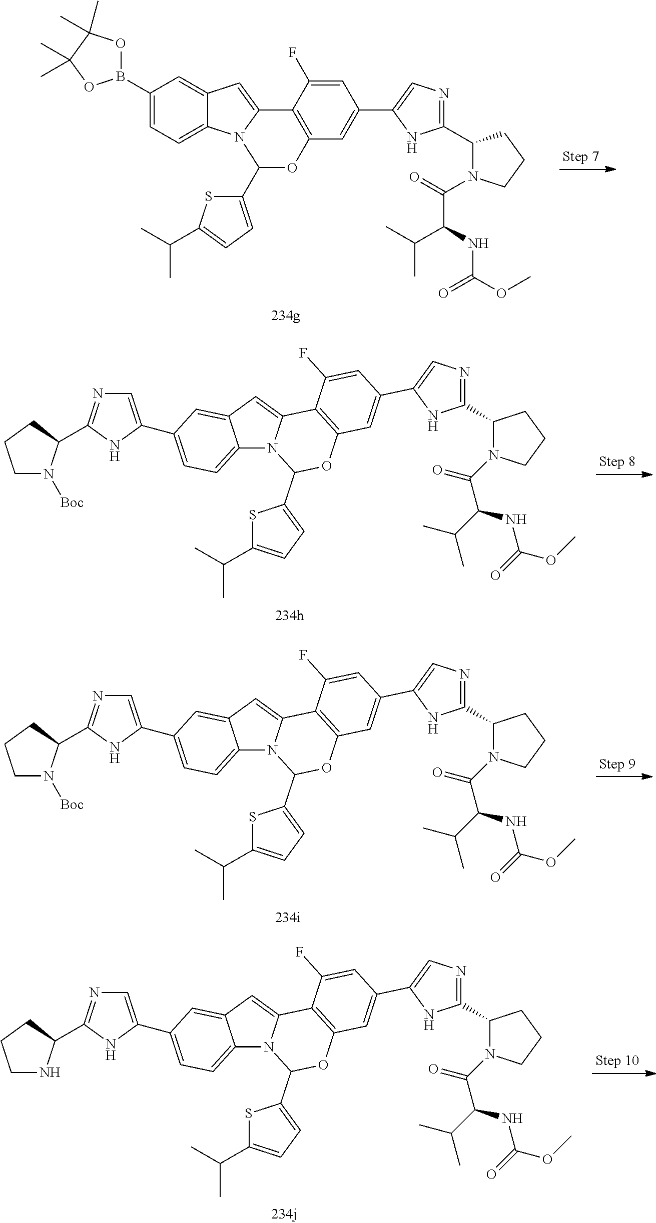

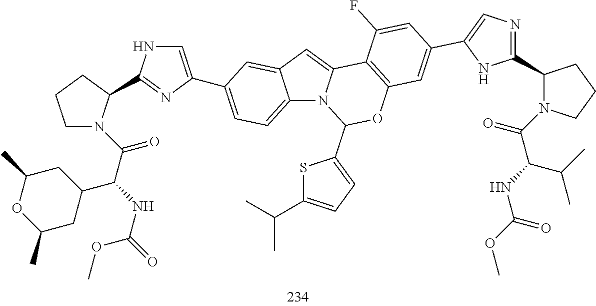 [SMILES: COC(=O)N[C@H](C(=O)N1CCC[C@@H]1c1ncc(-c2cc(F)c3c(c2)OC(c2ccc(C(C)C)s2)n2c-3cc3cc(-c4c[nH]c([C@@H]5CCCN5C(=O)[C@H](NC(=O)OC)C5C[C@@H](C)O[C@@H](C)C5)n4)ccc32)[nH]1)C(C)C]

### Step 1

Compound 36b was made in Example 40. To a mixture of 234a (5 g, 15 mmol) and core 3 (4.56 g, 30 mmol) in anhydrous CH₃CN (100 mL) was added TFA (0.5 mL) at 20° C. The mixture was allowed to stir at 20° C. for 6 hours. After filtration, the collected solid was washed with cold CH₃CN to provide 234b (5 g, 69.4% yield).

### Step 2

The solution of 234b (5 g, 10 mmol) in dry toluene (100 mL) was added DDQ (4.8 g, 20 mmol). After refluxing for 2 hours, the solvent was removed in vacuo and diluted with EtOAc. The organic layer was washed with saturated Na₂SO₃ solution and brine, dried over Na₂SO₄. After filtration and concentration, the residue obtained was washed with MeOH (10 mL) to provide 234c (5 g, 92.6% yield).

### Step 3

A suspension of 234c (5 g, 10 mmol), bis(pinacolato)diboron (3.04 g, 12 mmol), KOAc (2.94 g, 30 mmol) and Pd(dppf)Cl₂ (0.73 g, 1 mmol) in dioxane (100 mL) was allowed to stir at 100° C. under N₂ atmosphere for about 15 hours. After cooling to 20° C. and concentration, the residue obtained was purified using Flash column chromatography on silica gel (100 g, EtOAc/Hexane 0% to 10%) to provide 234d (4 g, 76.5% yield).

### Step 4

A suspension of 234d (4 g, 7.6 mmol), cap 31 (3.14 g, 8.4 mmol), Pd(dppf)Cl₂ (0.55 g, 0.76 mmol), Na₂CO₃ (2.4 g, 22.8 mmol) and in THF/H₂O (5:1, 120 mL) was refluxed at 80° C. for about 15 hours under N₂ atmosphere. After filtration and concentration, the residue obtained was purified using Flash column chromatography on silica gel (100 g, EtOAc/Hexane 10% to 100%) to provide 234e (3.8 g, 71.7% yield). LC/MS: Anal. Calcd. For [M+H]⁺ C36H35ClFN5O4S; 688.21. found 688.

### Step 5

To a mixture of 234e (3.8 g, 5.5 mmol), bis(pinacolato)diboron (1.8 g, 7.1 mmol), KOAc (1.62 g, 16.5 mmol), Pd₂(dba)₃ (0.5 g, 0.55 mmol), X-Phos (0.26 g, 0.55 mmol) degassed and sealed under N₂ was added dry dioxane. The mixture was allowed to stir at 100° C. for about 15 hours. After cooling to room temperature, the residue obtained was purified using Flash column chromatography on silica gel (80 g, DCM/MeOH 0% to 5%) to provide 234f (3.5 g, 81.4% yield).

### Step 6

To a stirred solution of 234f (3.5 g, 4.5 mmol) in methanol (100 mL) was added Pd/C (300 mg). The mixture was allowed to stir under 45 Psi H₂ pressure at 45° C. for about 15 hours. After filtration, the solvent was removed to provide 234g (2.8 g, 74.3% yield).

### Step 7

A suspension of 234g (2.8 g, 3.6 mmol), cap 31a (1.42 g, 4.3 mmol), Pd(dppf)Cl₂ (0.26 g, 0.36 mmol), Na₂CO₃ (1.1 g, 10.8 mmol) and in THF/H₂O (5:1, 60 mL) was refluxed at 80° C. for about 15 hours under N₂ atmosphere. Then the reaction mixture was poured into water, and extracted with ethyl acetate. The organic layer was dried over Na₂SO₄. After filtration and concentration, the residue obtained was purified using Flash column chromatography on silica gel (30 g, DCM/MeOH 0% to 5%) to provide 234h (2 g, 62.5% yield).

### Step 8

The compound of 234h (2 g) was separated by SFC by using the following conditions to provide 234i (0.8 g, 75% yield).

Column: OZ-H 250 mm*50 mm, 10 um

Solvent: Supercritical CO₂, B: MeOH (0.05% DEA), A:B=50:50

Flow rate: 220 mL/min

Wavelength: 220 nm

### Step 9

The compound of 234i (0.8 g, 0.89 mmol) was added into HCl/CH₃OH (20 mL). Then the mixture was allowed to stir at 20° C. for 3 hours. When the reaction completed, the mixture was concentrated in vacuo to provide 234j (0.75 g, 99.7% yield).

### Step 10

To a mixture of 234j (0.13 g, 0.16 mmol), cap 4 (0.04 g, 0.16 mmol) and DIPEA (0.2 mL) in DMF (3 mL) was added HATU (0.063 g, 0.16 mmol). The resulting mixture was allowed to stir at room temperature for about 15 hours, and then was subjected directly to RPLC to provide 234 (40 mg, 25%). ¹H NMR (MeOD) δ: 7.75-8.00 (m, 4H), 7.28-7.53 (m, 4H), 7.15 (m, 1H), 6.48-6.56 (m, 2H), 5.17-5.22 (m, 2H), 4.08-4.24 (m, 4H), 3.81-3.85 (m, 2H), 3.63-3.64 (m, 6H), 3.39 (m, 3H), 2.97-3.01 (s, 1H), 2.47-2.56 (m, 2H), 1.97-2.25 (m, 10H), 1.55-1.58 (m, 1H), 0.91-1.27 (m, 18H). LC/MS: Anal. Calcd. For [M+H]⁺ C54H64FN9O8S; 1018.46. found 1018.8.

### Example 72

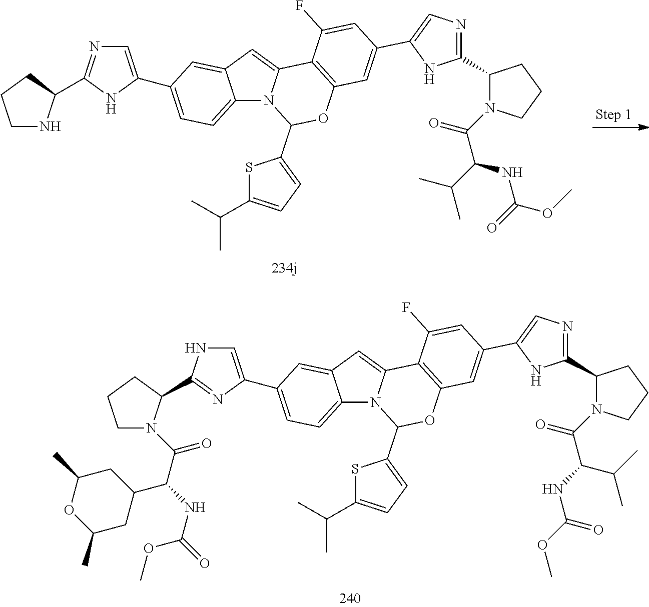

Compound 234j was prepared in Example 71. To a mixture of 234j (0.13 g, 0.16 mmol), cap 4 (0.04 g, 0.16 mmol) and DIPEA (0.2 mL) in DMF (3 mL) was added HATU (63 mg, 0.16 mmol). The resulting mixture was allowed to stir at 20° C. for about 15 hours, and then was subjected directly to RPLC to provide 240 (40 mg, 25% yield). ¹H NMR (MeOD) δ: 8.04 (br, 1H), 7.77-7.92 (m, 3H), 7.47-7.59 (m, 2H), 7.31-7.41 (m, 2H), 7.19 (br, 1H), 6.48-6.60 (m, 2H), 5.17-5.27 (m, 2H), 4.05-4.22 (m, 4H), 3.85 (s, 2H), 3.58-3.73 (m, 6H), 3.39 (d, J=8.6 Hz, 1H), 2.97-3.05 (m, 2H), 2.55 (m, 2H), 1.93-2.32 (m, 10H), 1.60 (m, 2H), 1.16-1.25 (m, 6H), 1.08 (m, 6H), 0.90 (m, 6H). LC/MS: Anal. Calcd. For [M+H]⁺ C54H64FN9O8S; 1018.46. found 1018.6.

### Example 73

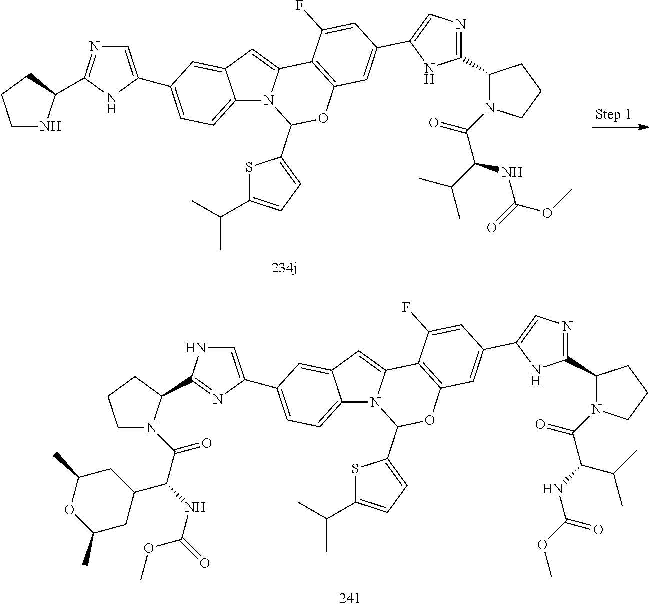

Compound 234j was prepared in Example 71. To a mixture of 234j (0.13 g, 0.16 mmol), isomer of cap 4 (0.04 g, 0.16 mmol) and DIPEA (0.2 mL) in DMF (3 mL) was added HATU (63 mg, 0.16 mmol). The resulting mixture was allowed to stir at 20° C. for about 15 hours, and then was subjected directly to RPLC to provide 241 (40 mg, 25%). ¹H NMR (MeOD) δ: 8.03 (br, 1H), 7.93 (s, 1H), 7.86 (s, 1H), 7.78 (s, 1H), 7.48-7.58 (m, 2H), 7.32-7.42 (m, 2H), 7.19 (br, 1H), 6.50-6.62 (m, 2H), 5.18-5.28 (m, 2H), 4.06-4.25 (m, 4H), 3.82-3.92 (m, 2H), 3.59-3.74 (m, 6H), 3.40 (d, J=8.6 Hz, 1H), 2.97-3.05 (m, 2H), 2.56 (m, 2H), 1.96-2.31 (m, 10H), 1.64 (m, 2H), 1.13-1.23 (m, 6H), 1.08 (m, 6H), 0.90 (m, 6H). LC/MS: Anal. Calcd. For [M+H]⁺ C54H64FN9O8S; 1018.46. found 1018.6.

### Example 74

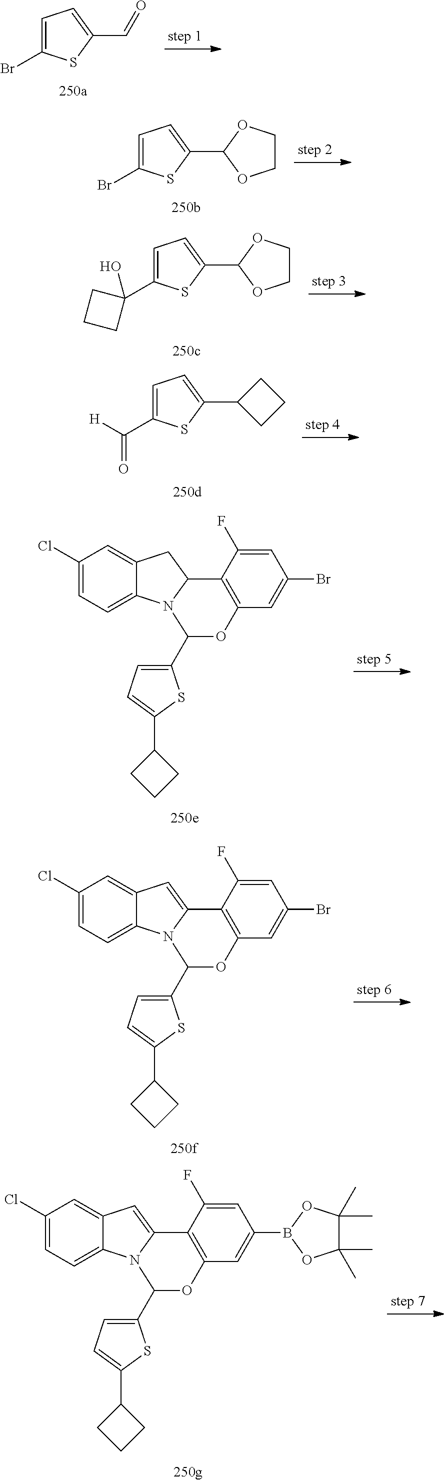

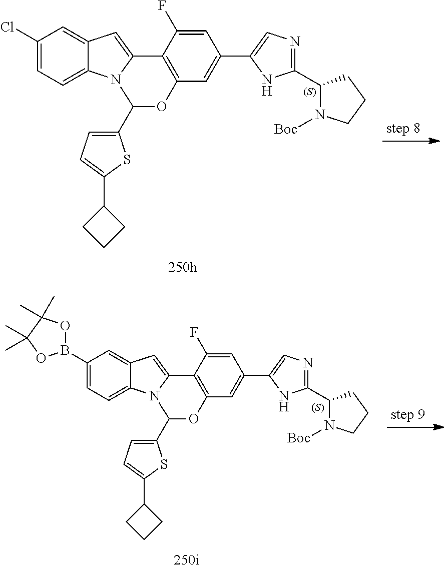

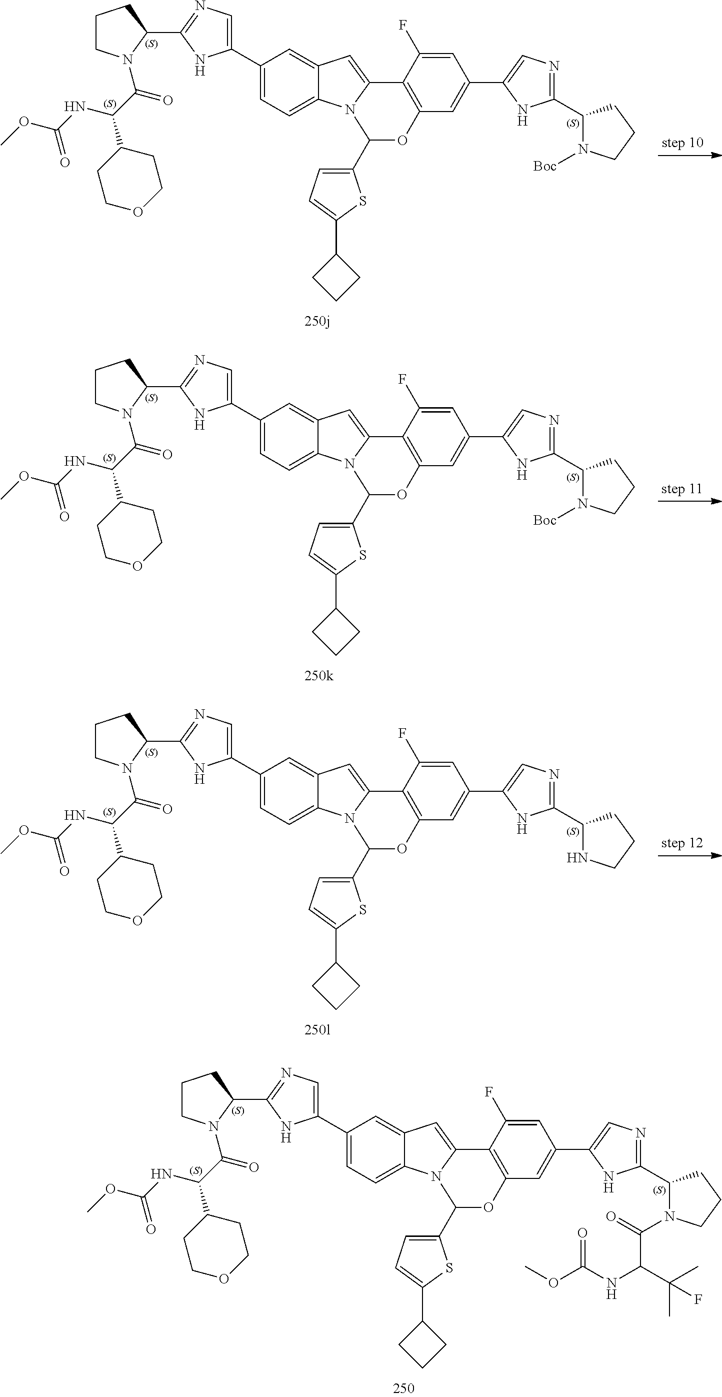

### Step 1

To a solution of 250a (19.6 g, 100 mmol) in toluene (250 mL) was added a glycol (9.3 g, 150 mmol) and PTSA (0.5 g). The reaction mixture was heated to reflux for about 15 hours with a Dean-stark Trap. After the mixture was cooled to 20° C., it was washed with saturated sodium carbonate and brine, dried over Na₂SO₄. The solvent was removed by evaporation to provide 250b (23 g, 98% yield).

### Step 2

To a solution of 250b (2.35 g, 10 mmol) in THF (20 mL) at −78° C. under N₂ atmosphere was added nBuLi (4.4 mL, 10 mmol). The mixture was allowed to stir for 10 minutes and then cyclobutanone (0.7 g, 10 mmol) was added dropwise. The reaction mixture was allowed to stir at the same temperature for half hour and then quenched with saturated ammonium chloride, extracted with ethyl acetate. The organic layer was dried over Na₂SO₄, filtered and concentrated in vacuo to provide 250c (1.95 g, 86% yield).

### Step 3

To the mixture of 250c (1.95 g, 8.6 mmol) and Et₃SiH (1.5 g, 13 mmol) in dichloromethane (40 mL) was added TFA (1.14 g) at 0° C. The mixture was allowed to stir at 45-50° C. for 2 hours before quenched with aqueous NaHCO₃. The organic layer was dried over Na₂SO₄, filtered and concentrated in vacuo. The residue obtained was purified using Flash column chromatography on silica gel (30 g, EtOAc/Hexane 0% to 2%) to provide 250d (0.55 g, 55% yield).

### Step 4

To a mixture of 250d (2 g, 12 mmol) and core 1 (3.44 g, 10 mmol) in anhydrous CH₃CN (15 mL) was added TFA (0.3 mL) at 20° C. The mixture was allowed to stir for 6 hours at 20° C. before filtrated. The solid was washed with CH₃CN to provide 250e (3.3 g, 67% yield).

### Step 5

The solution of 250e (3.3 g, 6.74 mmol) in dry toluene (30 mL) was added DDQ (2.27 g, 10 mmol). After refluxing for 2 hours, the solvent was removed in vacuo and diluted with EtOAc. The organic layer was washed with saturated Na₂SO₃ solution and brine, dried over Na₂SO₄. After filtration and concentration, the solid was washed with cold MeOH (20 mL) to provide 250f (3 g, 90% yield).

### Step 6

To a solution of 250f (3 g, 6.12 mmol) in dioxane (100 mL) was added bispinacol borate (1.86 g, 7.3 mmol) and Pd(dppf)Cl₂ (140 mg, 0.19 mmol) and KOAc (1.5 g, 15.2 mmol). The reaction mixture was allowed to stir under N₂ and heated to 110° C. for 4 hours. After cooling to 20° C. and concentration, the residue obtained was purified using Flash column chromatography on silica gel (50 g, EtOAc/Hexane 0% to 20%) to provide 250g (3.1 g, 94% yield).

### Step 7

A mixture of 250g (3.1 g, 5.8 mmol), cap 31a (2.19 g, 6.19 mmol), Pd(dppf)₂Cl₂ (420 mg, 0.58 mmol) and Na₂CO₃ (1.23 g, 11.6 mmol) in THF/H20 (5:1, 120 mL) was refluxed for about 15 hours under N₂ atmosphere. Then the reaction mixture was poured into water, and extracted with ethyl acetate. The organic layer was dried over Na₂SO₄. After filtration and concentration, the residue obtained was purified using Flash column chromatography on silica gel (10 g, EtOAc/Hexane 20% to 40%) to provide 250h (2.9 g, 77% yield). LC/MS: Anal. Calcd. For [M+H]⁺ C35H34ClFN4O3S; 645.20. found 645.4.

### Step 8

To a mixture of 250h (3 g, 4.65 mmol), bis(pinacolato)diboron (1.3 g, 5.1 mmol), KOAc (0.95 g, 10 mmol), Pd₂(dba)₃ (212 mg, 0.023 mmol), X-Phos (221 mg, 0.046 mmol) degassed and sealed under N₂ was added dry dioxane. The mixture was allowed to stir at 100° C. for about 15 hours. After cooling to 20° C., the residue obtained was purified using Flash column chromatography on silica gel (40 g, DCM/MeOH 0% to 5%) to provide 250i (2.9 g, 85.3% yield).

### Step 9

A suspension of 250i (3 g, 4.0 mmol), cap 32 (1.7 g, 4.0 mmol), Pd(dppf)Cl₂ (292 mg, 0.4 mmol) and Na₂CO₃ (0.86 g, 8 mmol) in THF/H₂O (5:1, 24 mL) was refluxed for about 15 hours under N₂ atmosphere. Then the reaction mixture was poured into water, and extracted with ethyl acetate. The organic layer was dried over Na₂SO₄. After filtration and concentration, the residue obtained was purified using Flash column chromatography on silica gel (4 g, DCM/MeOH 0% to 20%) to provide 250j (2 g, 54% yield). LC/MS: Anal. Calcd. For [M+H]⁺ C51H57FN8O7S: 945.41. found 945.6.

### Step 10

The compound of 250j (3 g, 4.0 mmol) was separated by SFC by using the following conditions to provide 250k (1 g, 67% yield).

Column: OD-3

Solvent: EtOH (0.05% DEA)

Flow rate: 2.5

### Step 11

Compound 250k (700 mg, 0.74 mmol) was added into HCl/dioxane (15 mL). Then the mixture was allowed to stir at 20° C. for 2 hours. When the reaction completed, the mixture was concentrated in vacuo to provide 2501 (600 mg, 91% yield).

### Step 12

To a mixture of 2501 (150 mg, 0.178 mmol), cap 1 and HATU (67.5 mg, 0.178 mmol) in DMF (30 mL) was added DIPEA (100 mg, 0.77 mmol). The resulting mixture was allowed to stir at 20° C. for 16 hours before the solution was subjected directly to RPLC to provide 250 (50 mg, 27.5%). ¹H NMR (MeOD) δ: 7.99 (s, 1H), 7.89 (s, 1H), 7.81 (s, 1H), 7.73 (s, 1H), 7.38-7.17 (m, 5H), 6.56 (d, J=3.6 Hz, 1H), 6.52 (d, J=3.6 Hz, 1H), 5.33 (m, 2H), 5.23 (m, 2H), 4.71 (d, J=7.2 Hz, 1H), 4.28 (d, J=8.0 Hz, 1H), 3.93-3.88 (m, 6H), 3.68 (s, 3H), 3.66 (s, 3H), 3.58-3.36 (m, 3H), 2.57-1.24 (m, 25H). LC/MS: Anal. Calcd. For [M+H]⁺ C53H59F2N9O8S; 1020.42. found 1020.8.

### Example 75

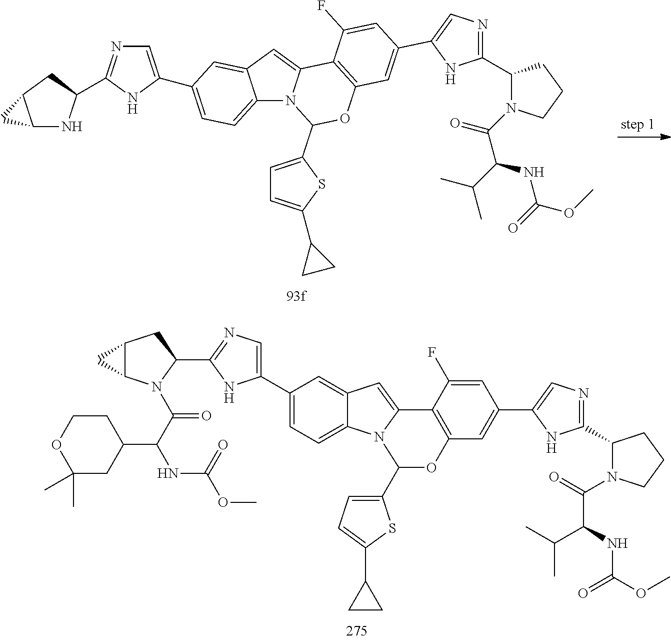

Compound 93f was made in Example 54. To a mixture of 93f (120 mg, 0.15 mmol), cap 5 (37 mg, 0.15 mmol) and HATU (57 mg, 0.15 mmol) in DMF (4 mL) was added DIEA (39 mg, 0.30 mmol). The resulting mixture was allowed to stir at 20° C. for about 15 hours, and then was subjected directly to RPLC to provide 275 (40 mg, 35% yield). ¹H NMR (MeOD) δ: 8.02 (s, 1H), 7.86 (s, 1H), 7.78 (d, J=12.0 Hz, 2H), 7.52-7.57 (m, 1H), 7.46-7.51 (m, 1H), 7.38 (d, J=11.2 Hz, 1H), 7.30 (s, 1H), 7.17 (d, J=2.4 Hz, 1H), 6.46-6.55 (m, 2H), 5.22 (t, J=7.2 Hz, 1H), 5.11-5.18 (m, 1H), 4.52 (d, J=7.6 Hz, 1H), 4.25 (d, J=7.6 Hz, 1H), 4.10 (m, 1H), 3.79-3.93 (m, 2H), 3.65-3.74 (m, 7H), 2.65-2.73 (m, 1H), 2.46-2.59 (m, 2H), 2.03-2.33 (m, 6H), 1.92-2.00 (m, 1H), 1.61 (m, 1H), 1.26-1.45 (m, 3H), 1.17 (m, 7H), 0.83-1.03 (m, 9H), 0.53-0.59 (m, 2H). LC/MS: Anal. Calcd. For [M+H]⁺ C55H62FN9O8S; 1028.44. found 1028.6.

### Example 76

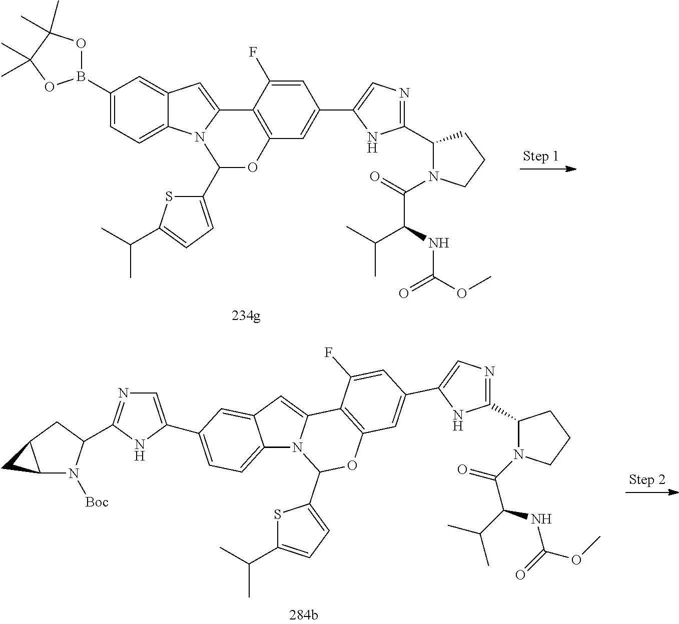

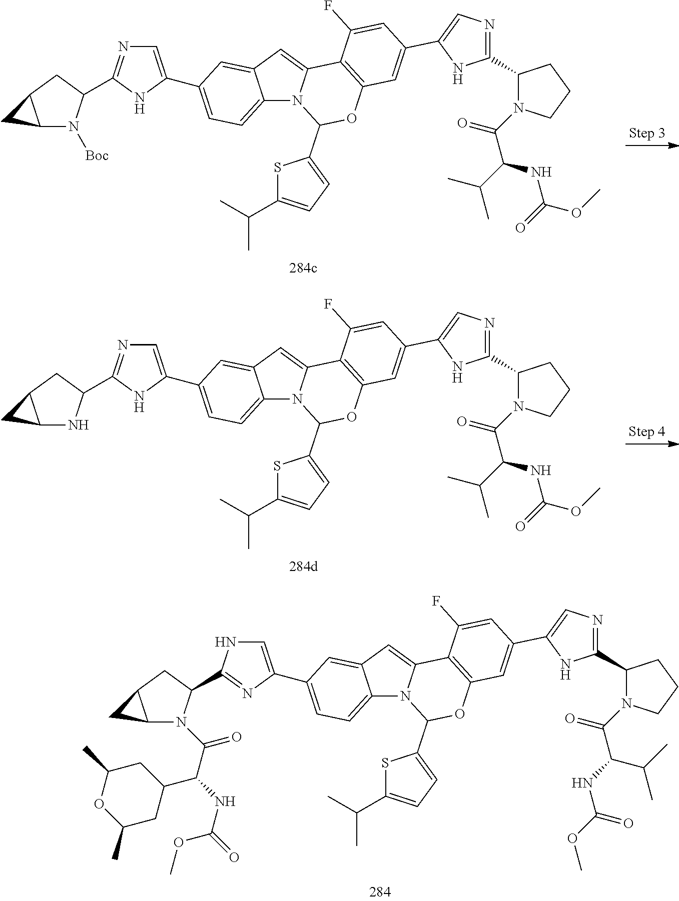

### Step 1

Compound 234g was prepared in Example 71. A suspension of 234g (2.8 g, 3.6 mmol), cap 32a (1.42 g, 4.3 mmol), Pd(dppf)Cl₂ (0.26 g, 0.36 mmol), Na₂CO₃ (1.1 g, 10.8 mmol) and in THF/H₂O (5:1, 60 mL) was refluxed for about 15 hours under N₂ atmosphere. Then the reaction mixture was poured into water, and extracted with ethyl acetate. The organic layer was dried over Na₂SO₄. After filtration and concentration, the residue obtained was purified using Flash column chromatography on silica gel (4 g, DCM/MeOH 0% to 5%) to provide 284b (2.1 g, 62.5% yield). LC/MS: Anal. Calcd. For [M+H]⁺ C49H55 FN8O6S; 903.39. found 903.6.

### Step 2

The compound of 284b (2.1 g) was separated by SFC by using the following conditions to provide 284c (0.8 g, 75% yield).

Column: OD-3

Solvent: Supercritical CO₂, B: MeOH (0.05% DEA)

### Step 3

The solution of 284c (0.8 g, 0.89 mmol) in HCl/CH₃OH (20 mL) was allowed to stir at room temperature for 2 hours. When the reaction completed, the mixture was concentrated in vacuo to provide the crude 284d (0.75 g, 99.7% yield). LC/MS: Anal. Calcd. For [M+H]⁺ C45H51FN8O4S; 803.39. found 803.6.

### Step 4

To a mixture of 284d (0.13 g, 0.16 mmol), cap 2 (0.04 g, 0.16 mmol) and DIPEA (0.2 mL) in DMF (3 mL) was added HATU (0.063 g, 0.16 mmol). The resulting mixture was allowed to stir at room temperature for about 15 hours, and then was subjected directly to RPLC to provide 284 (50 mg, 41.7% yield). ¹H NMR (MeOD) δ: 7.88 (br, 1H), 7.64 (br, 1H), 7.46 (d, J=9.0 Hz, 1H), 7.38 (s, 1H), 7.16-7.30 (m, 4H), 6.97 (m, 1H), 6.53 (d, J=3.1 Hz, 1H), 6.42 (d, J=3.1 Hz, 1H), 5.07-5.17 (m, 2H), 4.60 (d, J=7.4 Hz, 1H), 4.20 (d, J=7.0 Hz, 1H), 3.81-4.02 (m, 3H), 3.60-3.72 (m, 6H), 3.38-3.48 (m, 3H), 2.99 (dd, J=13.3, 6.7 Hz, 2H), 1.92-2.52 (m, 12H), 1.44-1.64 (m, 3H), 1.27 (s, 2H), 1.04-1.19 (m, 10H), 0.87-0.99 (m, 6H), 0.76 (m, 2H). LC/MS: Anal. Calcd. For [M+H]⁺ C55H64FN9O8S; 1030.46. found 1030.8.

### Example 77

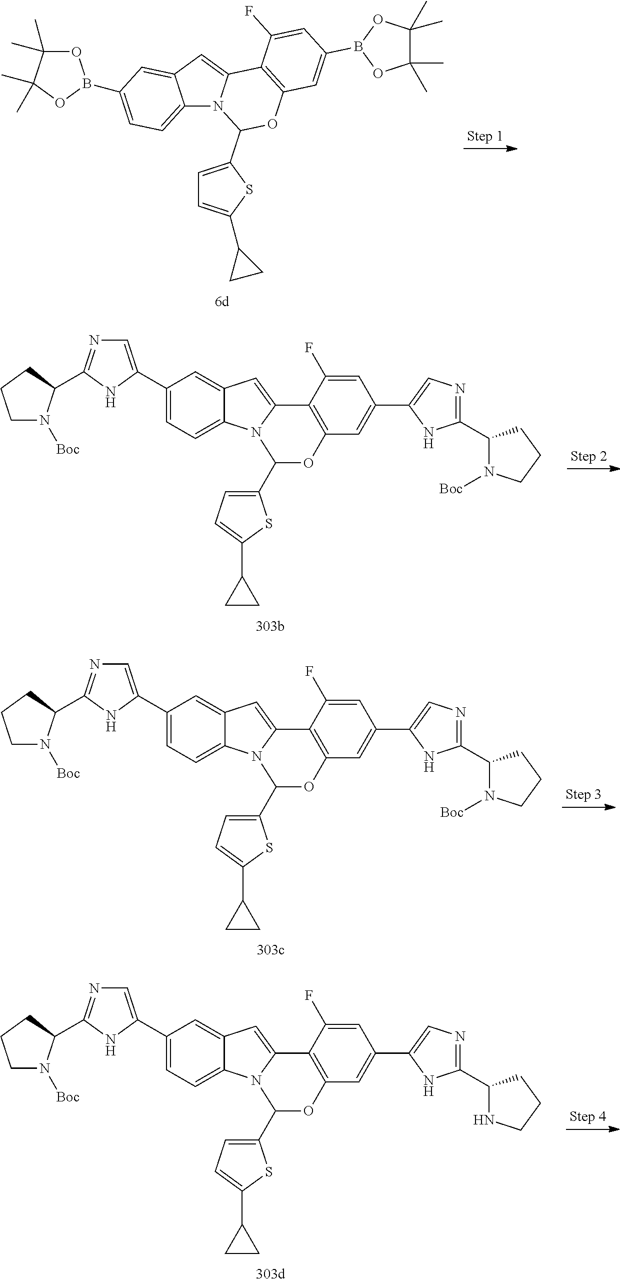

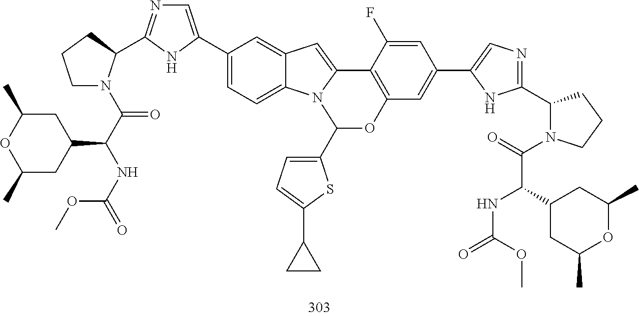 [SMILES: COC(=O)N[C@H](C(=O)N1CCC[C@H]1c1ncc(-c2cc(F)c3c(c2)OC(c2ccc(C4CC4)s2)n2c-3cc3cc(-c4cnc([C@@H]5CCCN5C(=O)[C@@H](NC(=O)OC)C5C[C@@H](C)O[C@@H](C)C5)[nH]4)ccc32)[nH]1)C1C[C@@H](C)O[C@@H](C)C1]

### Step 1

Compound 6d was made in Example 35. The compound 6d (2.5 g, 4.07 mmol), cap 31a (2.58 g, 8.16 mmol), Pd(dppf)Cl₂ (0.26 mg, 0.9 mmol) and Na₂CO₃ (1.73 g, 16.3 mmol) in THF/H₂O (64 mL/8 mL) was refluxed for about 15 hours under N₂ atmosphere. After filtration, the filtrate was washed with water (50 mL) and extracted with ethyl acetate (100 mL). The organic layer was washed with brine and dried over Na₂SO₄. After filtration and concentration, the residue obtained was purified using Flash column chromatography on silica gel (40 g, Hexane/EtOAc 20% to 50%) to provide 303b (2.4 g, 70% yield). LC/MS: Anal. Calcd. For [M+H]⁺ C46H50FN7O5S; 832.36. found 832.6.

### Step 2

The compound of 303b (1.6 g) was separated by SFC by using the following conditions to provide 303c (0.6 g, 75%).

Column: OD-3 250×4.6 mm I.D., Sum

Solvent: 40% of iso-propanol (0.05% DEA) in CO₂

Flow rate: 2.5 mL/min

Wavelength: 220 nm

### Step 3

303c (0.6 g, 0.72 mmol) was added into HCl/dioxane (15 mL, 4M) and the mixture was allowed to stir at room temperature for 2 hours. When the reaction completed, the mixture was concentrated in vacuo to provide 303d (0.52 g, 100% yield). LC/MS: Anal. Calcd. For [M+H]⁺ C₃₆H₃₅FN₇OS; 632.25. found 632.4.

### Step 4

To a mixture of 303d (0.23 g, 0.36 mmol), cap 4 (0.18 g, 0.72 mmol) and DIPEA (0.3 mL) in DMF (7 mL) was added HATU (0.27 g, 0.72 mmol). The resulting mixture was allowed to stir at room temperature for 1 h. The mixture was purified using Pre-HPLC to provide 303 (0.17 g, 46% yield). ¹H NMR (MeOD) δ: 8.02 (s, 1H), 7.88 (s, 1H), 7.77 (s, 2H), 7.53 (d, J=8.0 Hz, 1H), 7.46 (d, J=8.0 Hz, 1H), 7.37 (d, J=8.0 Hz, 1H), 7.31 (s, 1H), 7.16 (s, 1H), 6.50 (d, J=4.0 Hz, 1H), 6.45 (d, J=4.0 Hz, 1H), 5.22-5.15 (m, 2H), 4.24-4.21 (m, 2H), 4.08-4.06 (m, 2H), 3.87-3.84 (m, 2H), 3.64 (s, 6H), 2.44-3.37 (m, 4H), 2.55-2.47 (m, 2H), 2.26-1.93 (m, 9H), 1.57-1.48 (m, 2H), 1.30-1.26 (m, 2H), 1.08-1.00 (m, 12H), 0.97-0.88 (m, 6H), 0.53 (d, J=4.0 Hz, 2H).

### Example 78

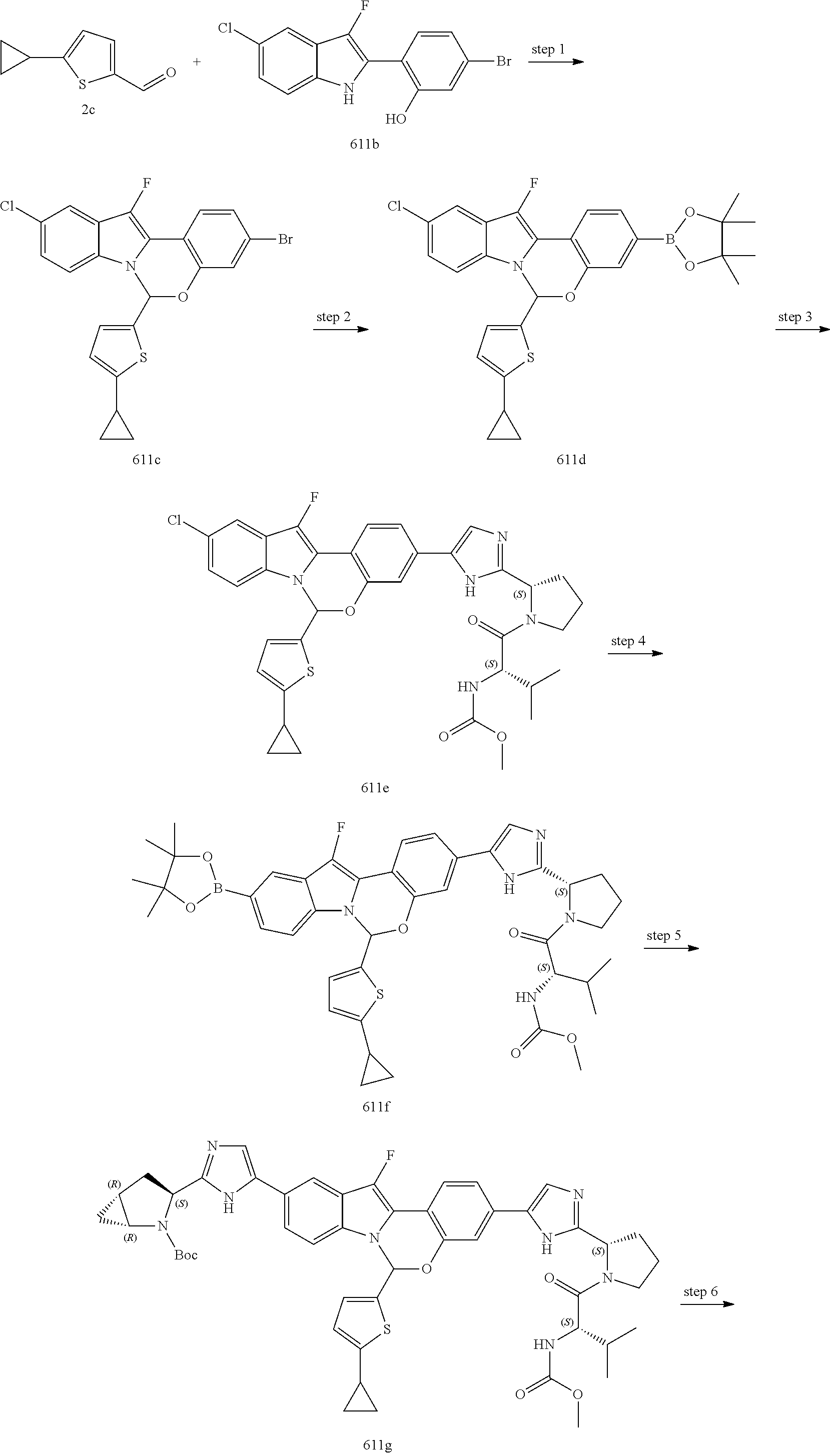

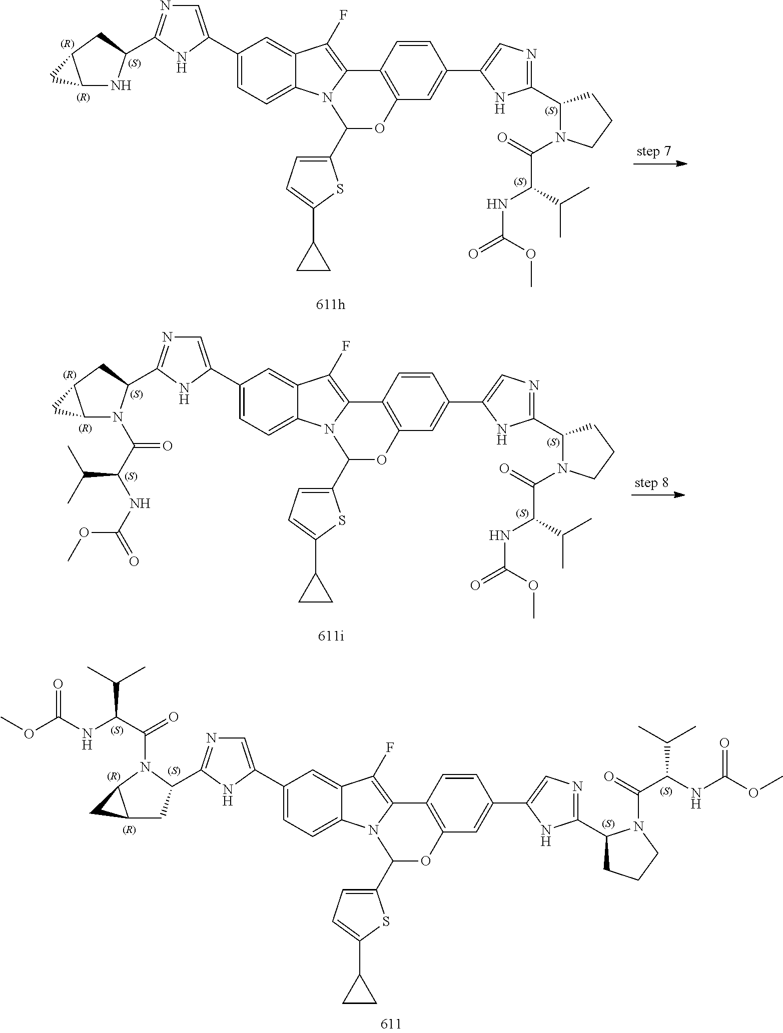

### Step 1

Compound 2c was made in Example 33. Compound 611b was made as described in Example 19 of International Publication No. 2012/040923 A1. To a mixture of 2c (0.516 g, 3.39 mmol) and 611b (1.05 g, 3.08 mmol) in anhydrous Toluene (10 mL) was added 4-methylbenzene-1-sulfonyl chloride (0.176 g, 0.925 mmol). The mixture was allowed to stir at 130° C. for 12 hours. The product 611c was separated by Flash LC in 100% Hexane to provide (0.4 g, 27.3% yield).

### Step 2

A suspension of 611c (0.4 g, 0.843 mmol), bis(pinacolato)diboron (0.214 g, 0.843 mmol), KOAc (0.248 g, 2.53 mmol) and Pd(dppf)Cl₂ (68.8 mg, 0.084 mmol) in dioxane (10 mL) in pressure tube was purged with nitrogen and vacuumed 2 times and stirred at 90° C. for 5 hours. The reaction mixture of the 611d was cooled and used as is in next step. LC/MS: Anal. Calcd. For [M+H]⁺ C28H26BClFNO3S: 521.14. found 522.2.

### Step 3

To the suspension of 611d, cap 31 (315 mg, 0.843 mmol), K₂CO₃ (350 mg, 2.53 mmol) and Pd(dppf)Cl₂ (103 mg, 0.126 mmol) in 10 mL Dioxane/1 mL H₂O in pressure tube was purged with nitrogen and vacuumed 2 times and stirred at 90° C. for 2 hours. After that, the mixture was washed with water and extracted with ethyl acetate, washed with brine and dried over anhydrous sodium sulfate. After being concentrated in vacuo, the residue obtained was purified using Flash column chromatography on silica gel, eluting with Ethyl acetate/Hexane (0% to 100%) to provide 450 mg 611e (450 g, 78% yield). LC/MS: Anal. Calcd. For [M+H]⁺ C36H35ClFN5O4S; 687.21. found 688.4.

### Step 4

A mixture of 611e (400 mg, 0.581 mmol), bis(pinacolato)diboron (148 mg, 0.581 mmol), KOAc (171 mg, 1.744 mmol), Pd₂(dba)₃ (120 mg, 0.116 mmol), X-Phos (83 mg 0.174 mmol) in the dry dioxane 15 ml was degassed and sealed under N₂. The mixture was allowed to stir at 110° C. for 7 hours. The reaction mixture of the 611f was cooled and used as is in next step. LC/MS: Anal. Calcd. For [M+H]⁺ C42H47BFN5O6S; 779.33. found 780.78.

### Step 5

A mixture of 611f from a previous step, cap 33a (191 mg, 0.581 mmol), K₂CO₃ (241 mg, 1.743 mmol) and Pd(dppf)Cl₂ (95 mg, 0.116 mmol) 10 mL Dioxane/1 mL H₂O in pressure tube was purged with nitrogen and vacuumed 2 times and stirred at 90° C. for 5 hours. After that, the mixture was washed with water and extracted with ethyl acetate, washed with brine and dried over anhydrous sodium sulfate. After being concentrated in vacuo, the residue obtained was purified using Flash column chromatography on silica gel, eluting with chromatography Ethyl acetate/Hexane (0% to 100%) to provide 300 mg 611g (300 g, 54% yield). LC/MS: Anal. Calcd. For [M+H]⁺ C49H53FN8O6S; 900.38. found 901.89.

### Step 6

To a solution of compound 611g (300 mg, 0.333 mmol) in dry dioxane (10 mL) was added HCl-dioxane 4M (1.665 mL) through syringe and stirred at 25° C. for 15 hours, then concentrated in vacuo and dried under high vacuum to provide HCl salt of the desired product compound 611h (303 mg, 100%). LC/MS: Anal. Calcd. For [M+H]⁺ C44H45FN8O4S; 800.33. found 801.79.

### Step 7

To a mixture of the compound 611h (151 mg, 0.166 mmol), cap 31c (29.1 mg, 0.166 mmol) and HATU (63.1 mg, 0.166 mmol) in DMF (5 mL), DIPEA (0.145 mL, 0.829 mmol) was added at 0° C. The resulting mixture was allowed to stir at 0° C. for 0.5 hour. After that, the mixture was washed with water and extracted with ethyl acetate, washed with brine and dried over anhydrous sodium sulfate. After being concentrated in vacuo, the residue obtained was purified using Flash column chromatography on silica gel, eluting with chromatography MeOH/CH₂Cl₂ (0% to 10%) to provide 63 mg 611i (63 mg, 40% yield). LC/MS: Anal. Calcd. For [M+H]⁺ C49H53FN8O6S; 957.40. found 958.98.

### Step 8

Compound 611 was obtained from compound 611g (0.125 g) by SFC by using the following conditions:

Column: AS, 21×250 mm,

Mobile phase: 40% IPA+0.2% DEA/CO₂

Flow rate: 70 mL/min

Back pressure: 100 bar

Column temperature: 35° C.

Wavelength: 254 nm

Compound 611 (54.3 mg, 38% yield).

LC/MS: Anal. Calcd. For [M+H]⁺ C49H53FN8O6S; 957.40. found 958.75.

The following compounds of the present invention were made using the methods described in the Example above and substituting the appropriate reactants or reagents.

| | | | Observed |
|---|---|---|---|
| ID | Structure | Isomer | [M + H]+ |
| 362 363 | 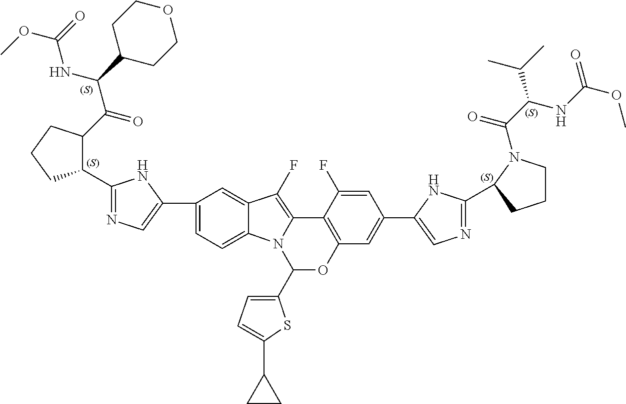 | Isomer 1 Isomer 2 | 1006.4 1006.4 |
| 406 | 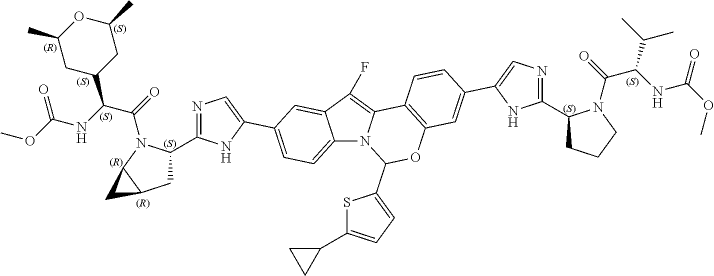 | Isomer 1 | 1028.2 |
| 413 | 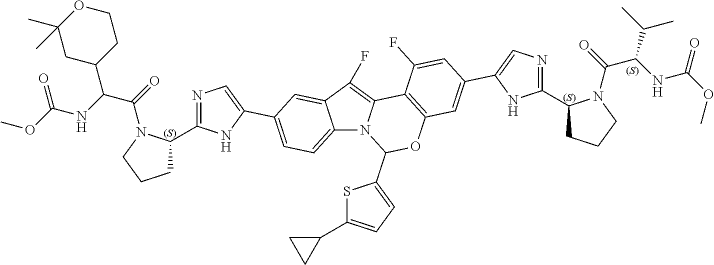 | Isomer 1 | 1034.2 |
| 422 423 | 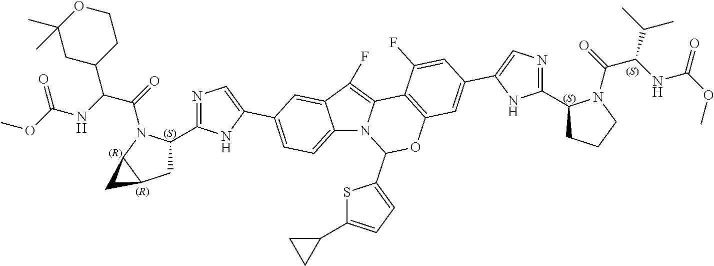 | Isomer 1 Isomer 2 | 1046.2 1046.2 |
| 601 602 603 | 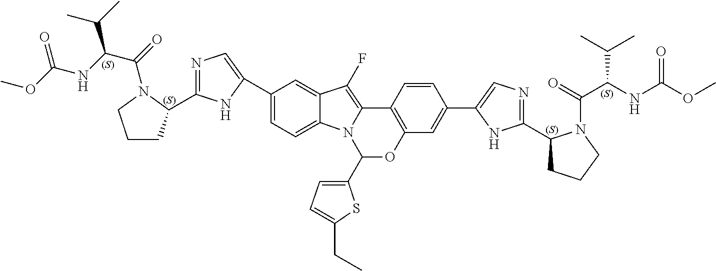 | Isomer 1 Isomer 2 racemic | 935.11 935.14 935.20 |
| 604 605 606 | 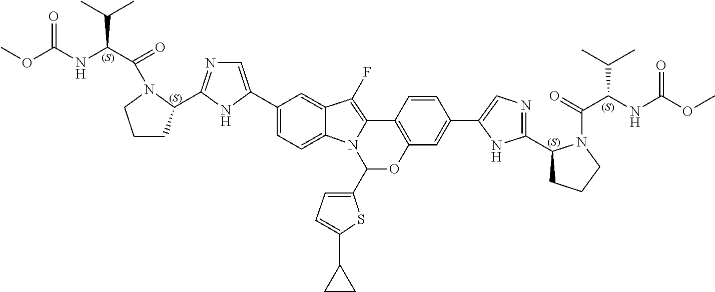 | Isomer 2 Isomer 1 racemic | 946.91 946.89 947.02 |
| 611 612 613 | 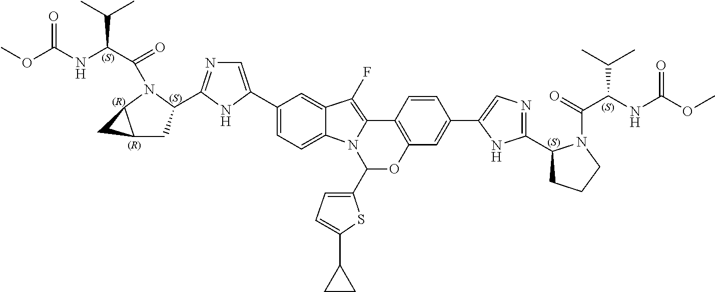 | Isomer 2 Isome 1 racemic | 958.75 958.70 958.95 |
| 633 634 635 | 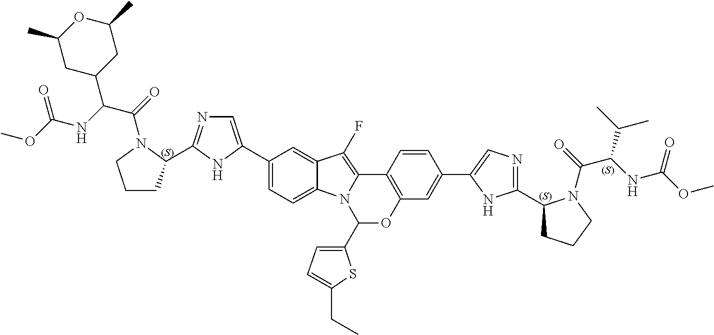 | Isomer 1 Isomer 2 racemic | 1005.18 1005.25 1004.74 |
| 637 638 639 | 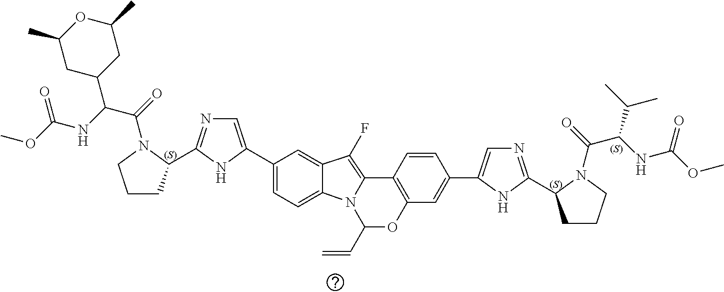 | Isomer 2 Isomer 1 racemic | 1017.31 1017.32 1017.14 |
| 643 644 645 | 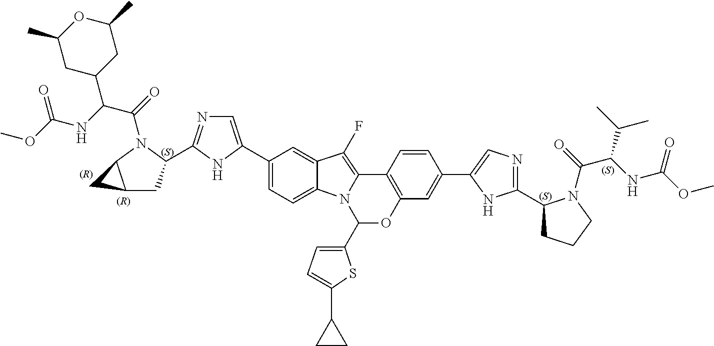 | Isomer 1 Isomer 2 racemic | 1029.15 1029.14 1029.16 |

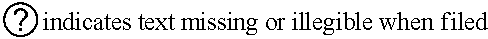

### Example 79

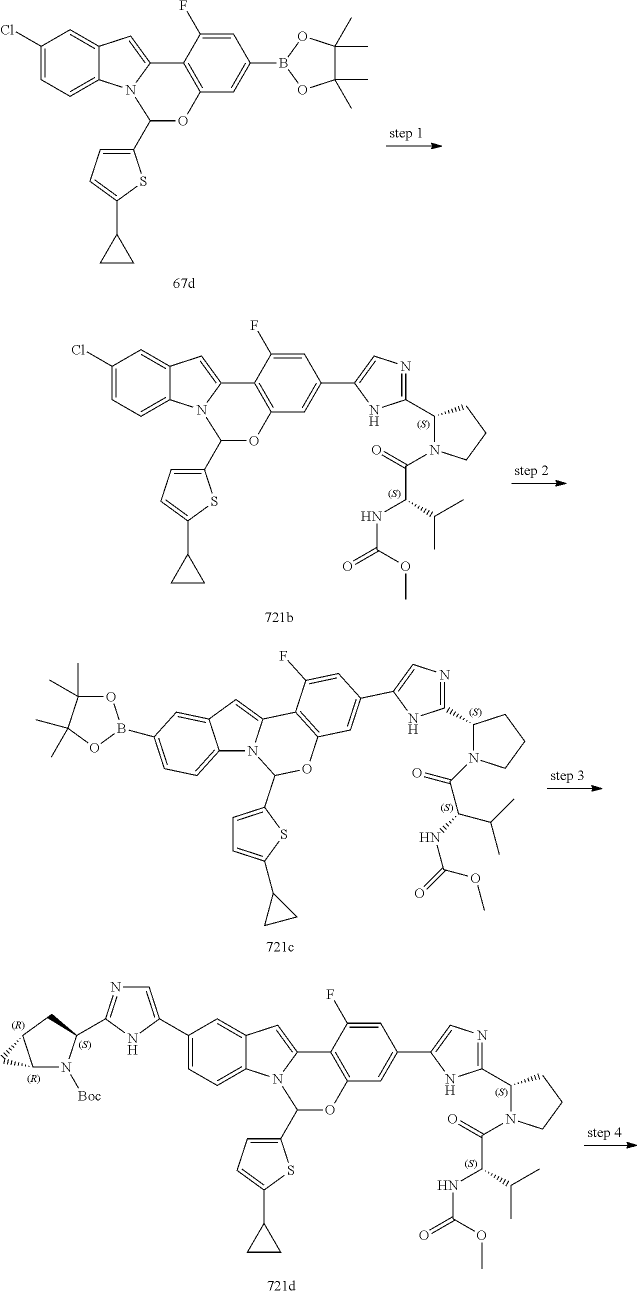

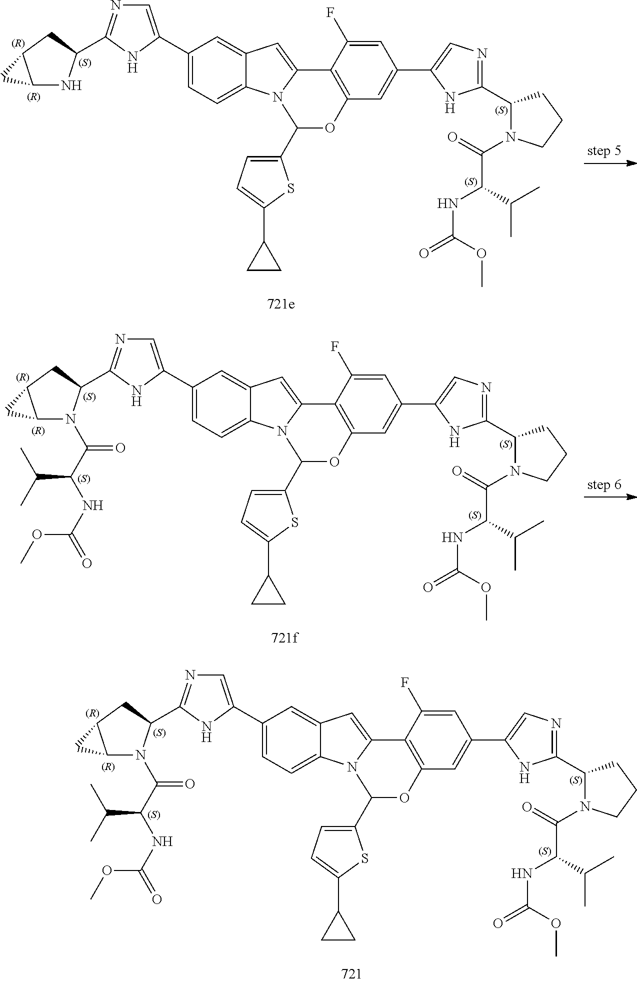

### Step 1

Compound 67d was prepared in Example 49. A suspension of the compound 67d (341 mg, 0.65 mmol), cap 31 (366 mg, 0.98 mmol), Pd(dppf)Cl₂ (107 mg, 0.13 mmol), K₂CO₃ (1.96 mL, 1 M, 1.96 mmol) and in dioxane (7 mL) was added to a sealed tube and degassed for three time before heated at 90° C. for about 15 hours. After cooling, the mixture was filtered; the filtrate was diluted with EtOAc (20 mL) and extracted with brine (30 mL). The organic layer was dried over anhydrous sodium sulfate. After being concentrated in vacuo, the residue obtained was purified on ISCO column (40 g) and eluted with EtOAc to provide compound 721b (315 mg, 0.46 mmol, 70% yield). LC/MS: Anal. Calcd. For [M+H]⁺ C36H35ClFN5O4S: 688.21. found 688.39.

### Step 2

A mixture of compound 721b (315 mg, 0.46 mmol), bis(pinacolato) diboron (174 mg, 0.69 mmol), KOAc (135 mg, 1.37 mmol), Pd₂(dba)₃ (47.4 mg, 0.046 mmol), X-Phos (43.6 mg, 0.092 mmol) and dioxane (3 mL) was added to a reaction tube. It was sealed and degassed. The mixture was allowed to stir at 110° C. for about 15 hours. Under standard work-up to provide the residue which was purified on a ISCO column eluted with EtOAc to provide compound 721c (355 mg, 0.46 mmol, 99% yield). LC/MS: Anal. Calcd. For [M+H]⁺ C42H47BFN5O6S: 780.33. found 780.46.

### Step 3

A suspension of the compound 721c (355 mg, 0.46 mmol), cap 33a (179 mg, 0.55 mmol), Pd(dppf)Cl₂ (74.4 mg, 0.091 mmol), K₂CO₃ (1.37 mL, 1 M, 1.37 mmol) and in dioxane (4 mL) was added to a sealed tube and degassed for three time before heated at 85° C. for about 15 hours. After cooling, the mixture was filtered; the filtrate was diluted with EtOAc (20 mL) and extracted with brine (30 mL). The organic layer was dried over anhydrous sodium sulfate. After being concentrated in vacuo, the residue obtained was purified on a ISCO column (24 g) and eluted with CH₂Cl₂: MeOH in NH₄OH (10:1) in CH₂Cl₂ (0% to 8%) to provide compound 721d (115 mg, 0.13 mmol, 28% yield). LC/MS: Anal. Calcd. For [M+H]⁺ C49H53FN8O6S; 901.38. found 901.54.

### Step 4

The compound 721d (115 mg, 0.13 mmol) was dissolved in dioxane (1 mL). Then HCl (0.32 mL, 4N in dioxane) was added. The mixture was allowed to stir at 25° C. for 2-3 hours. The mixture was concentrated in vacuo to provide the crude 721e (116 mg, 0.13 mmol, 100% yield).

### Step 5

To a mixture of the crude 721e (86 mg, 0.094 mmol), cap 31c (17.38 mg, 0.1 mmol), HATU (39.5 mg, 0.1 mmol) and DMF (1.5 mL) was added and DIPEA (73.3 mg, 0.57 mmol) at 0° C. The resulting mixture was then warmed to 25° C. and stirred for 2 hours. A few drops of water was added to quench the reaction and the crude material was purified on Gilson Reverse Phase HPLC to provide the desired compound 721f (90 mg, 99% yield).

### Step 6

Compound 721 was obtained from compound 721d (90 mg) by SFC separation by using the following conditions.

Column: AS, 21×250 mm,

Mobile phase: 40% MeOH+0.2% DEA,

Flow rate: 50 mL/min

Compound 721 (33 g, 34% yield). LC/MS: Anal. Calcd. For [M+H]⁺ C51H56FN9O7S; 958.4. found 958.66.

The following compounds of the present invention were made using the methods described in the Example above and substituting the appropriate reactants or reagents.

| | | | Observed |
|---|---|---|---|
| ID | Structure | Isomer | [M + H]+ |
| 719 720 721 | 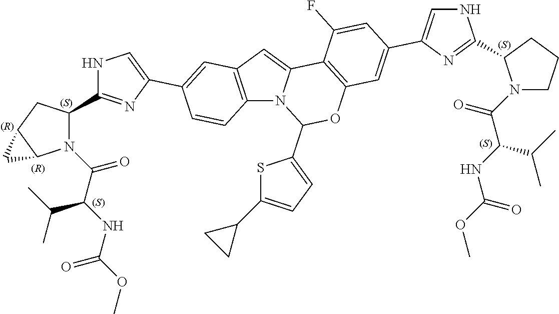 | racemic Isomer 1 Isomer 2 | 958.51 958.80 958.67 |
| 723 | 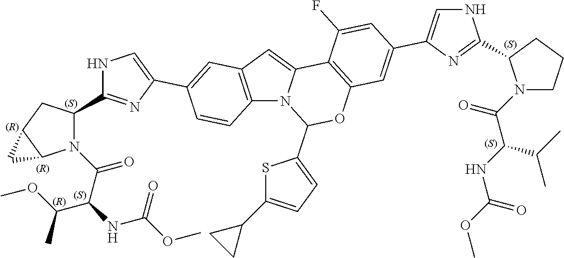 | racemic | NA |

### Example 80

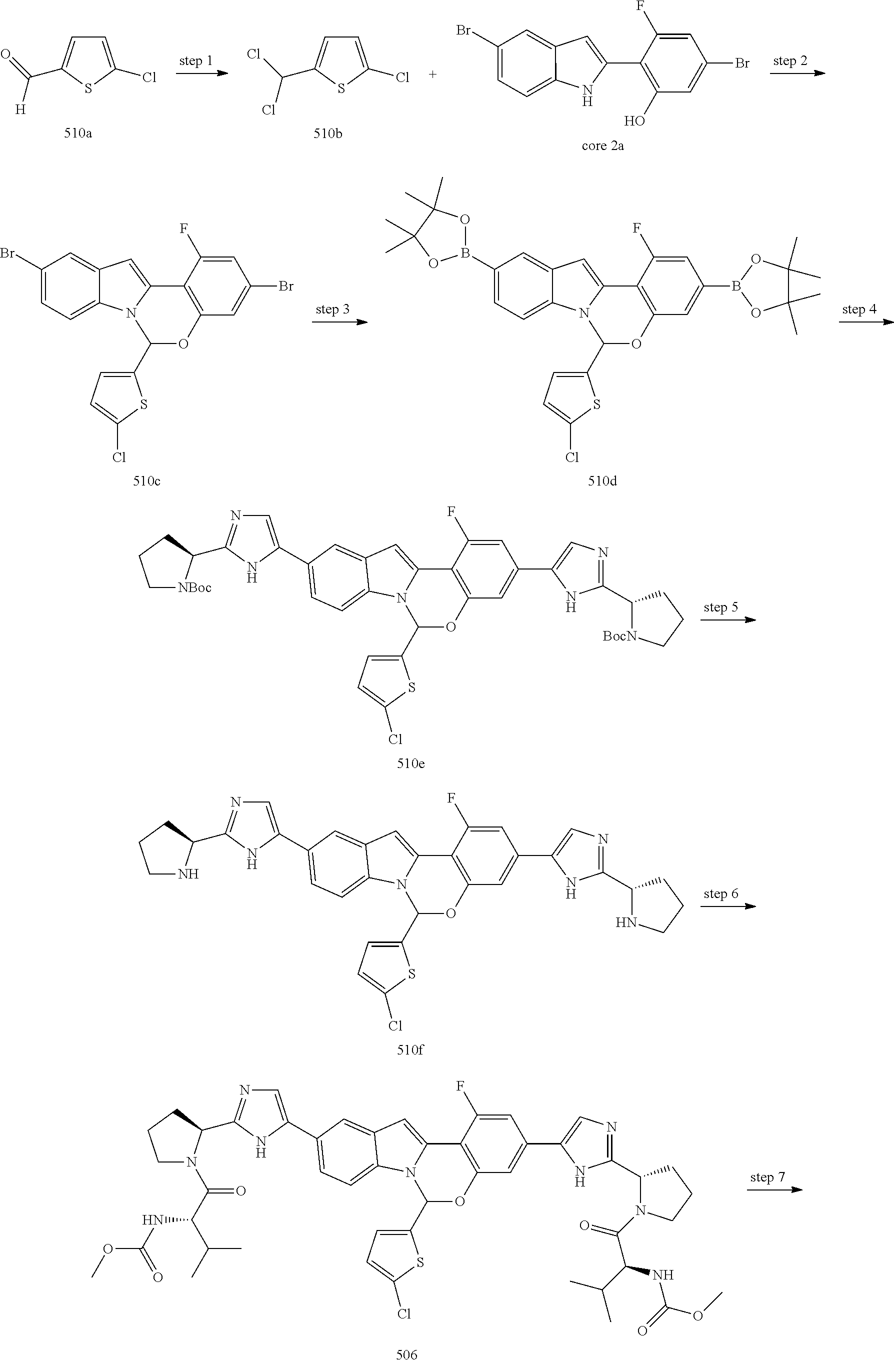

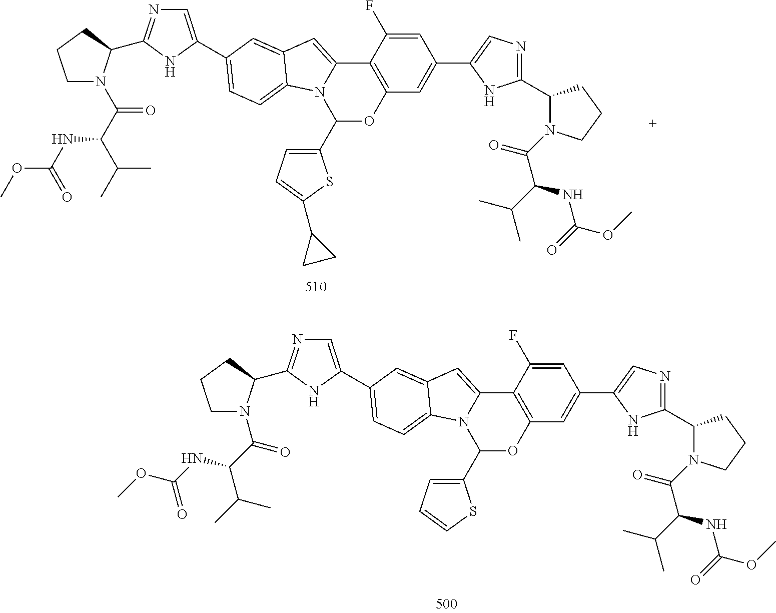

### Step 1

Pyridine (0.552 mL, 6.82 mmol) was added to a solution of 510a (5 g, 34.1 mmol) in CH₂Cl₂ (87 mL) at −10° C. (ice in acetone) followed by addition of PCl₅ (7.1 g, 34.1 mmol) all at once. The reaction was allowed to stir at −10° C. for another half hour. NaHCO₃ (8.6 g, 102 mmol) was added as solid to the reaction mixture. The mixture was allowed to stir another 15 min; then filtered through celite; washed with more CH₂Cl₂. The filtrate was concentrated in vacuo and purified using a short column (50 g) and the product 510b was isolated to provide ˜7.0 g oil 100% yield.

### Step 2

The core 2a (5 g, 13 mmol), 510b (5.23 g, 26 mmol) in 50 mL of DMSO, Cs₂CO₃ (19.04 g, 58.4 mmol) was added. The reaction was allowed to stir at 100° C. for one hon. Solid was filtered off and the filtrated was extracted with brine. The organic layer was dried and concentrated in vacuo. The crude material was purified with Isco 220 g silica gel column eluted with EtOAc in Hex 0% to 5% to provide product, which was repurified using Isco 120g silica gel column under the same condition to provide 510c (0.7 g, 10.5% yield)

### Step 3

510c (715 mg, 1.392 mmol), Pd(dppf)Cl₂ (227 mg, 0.278 mmol), KOAc (683 mg, 6.96 mmol), bis(pinacolato)diboron (707 mg, 0.278 mmol) and dioxane was added into a 20 mL microwavetube. The tube was vacuumed and flashed with N₂ for three times. The mixture was allowed to stir at 90° C. for 5 hours. The mixture was cooled, EtOAc (˜30 mL) was added and the mixture filtered through a silica pad washed with EtOAC (˜30 mL) the filtrate was combined and the solvent was evaporated under reduced pressure. The residue obtained was purified using column chromatography on silica gel (40 g column), eluting with 0-10% EtOAc in Hex to provide 510d as a yellow solid (0.7 g, 83% yield)

### Step 4

To the reaction mixture of 510d (0.68 g, 1.12 mmol) was added cap 31a (0.778 g, 2.462 mmol), Pd(dppf)Cl₂ (137 mg, 0.168 mmol), and 1M K₂CO₃ aq (5.6 mL). The mixture was sealed and degassed and stirred at 80° C. for about 15 hours. EtOAc (˜50 mL) and water was added and the mixture was filtered through a celite pad. The filtrate was separated and the aqueous phase was extracted with EtOAc (20 mL×2). The organic layer was combined and washed with brine, dried over Na₂SO₄, and concentrated in vacuo. The crude material was purified on a ISCO column (40 g) and eluted with EtOAc:Hex 0% to 50% then 80% to provide 510e (0.4 g, 43% yield)

### Step 5

To the DCM solution of 510e (400 mg, 0.474 mmol) was added HCl in dioxane (4M) solution (2 mL) and the reaction was allowed to stir at 20° C. for 0.5 hour. LCMS showed reaction was not done. Another 2.5 mL HCl was added and stirred at 20° C. for another 0.5 hour. LCMS showed reaction was done. The solvent was evaporated to dry to provide 510f, which was used in the next step without purification (0.36 g, 98% yield)

### Step 6

To the DMF solution of 510f (324.5 mg, 0.42 mmol), cap 31c (155 mg, 0.883 mmol), HATU (384 mg, 1.009 mmol) in DMF, was added dropwise TEA (255 mg, 2.52 mmol) at 0° C. (ice-water bath) the reaction mixture was allowed to stir at 0° C. for 40 min. LCMS showed reaction done. Water (˜20 mL) was added and the mixture was extracted with DCM (3×10 mL). The combined organic fractions were washed with brine (10 mL), dried (Na₂SO₄), filtered and the solvent was evaporated under reduced pressure. The residue obtained was purified using column chromatography on silica gel (40 g), eluting with 0-10% NH₃-MeOH in DCM) to provide impure product, the material was purified again on Pre-HPLC (0-80%/20 minutes, AcCN/H₂O) to provide 506 as desired product. (0.02 g, 4.7% yield)

### Step 7

506 (16 mg, 0.014 mmol), cyclopropyl boranic acid (11.76 mg, 0.137 mmol), Pd₂(dba)₃ (2.508 mg, 2.74 μmol), and X-PHOS (2.61 mg, 5.48 μmol) are added into a 10 mL sealed tube. After the flask was flashed with N₂, Dioxane (456 μl) and K₂CO₃ (82 μl, 0.082 mmol) was added. The mixture was allowed to stir at 110° C. for 16 hours. After cooling down, the solution was concentrated in vacuo and purified using Pre-HPLC (0-80% AcCN in H₂O/15 min) to provide 510 (7.4 mg, 46% yield) as desired product and 500 (2.8 mg, 18% yield) as dechlorinated product.

The following compounds of the present invention were made using the methods described in the Example above and substituting the appropriate reactants or reagents.

| | | | Observed |
|---|---|---|---|
| ID | Structure | Isomer | [M + H]+ |
| 500 | 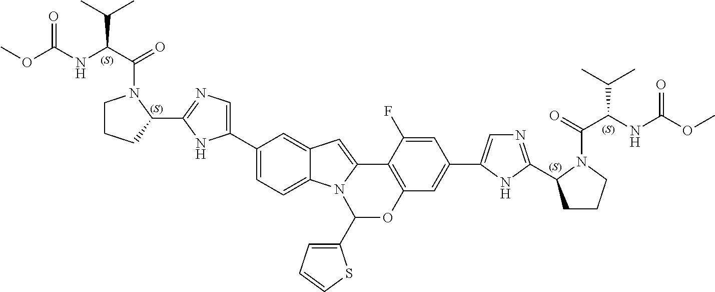 | racemic | 906.4 |
| 506 | 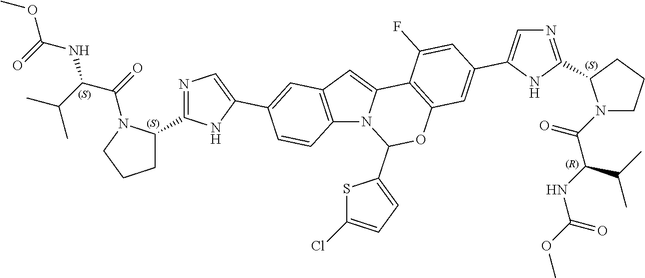 | racemic | 942.0 |
| 510 | 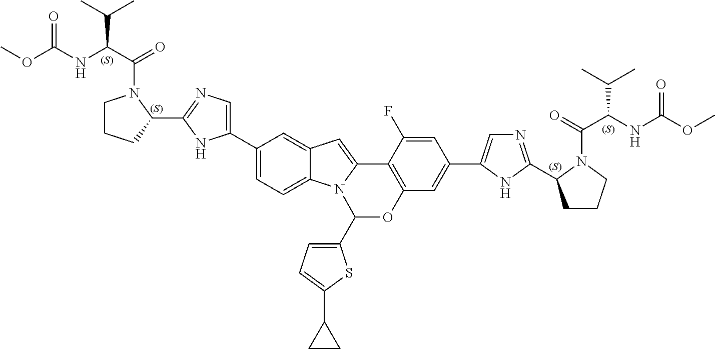 | Racemic | 946.4 |
| 700 701 702 | 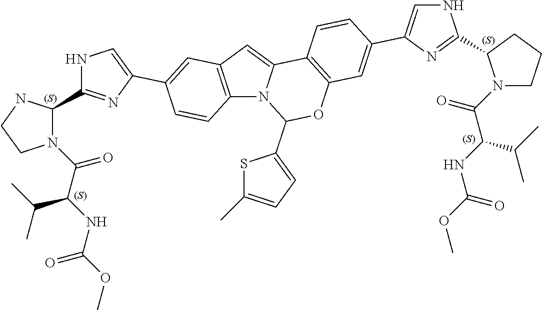 | racemic Isomer 1 Isomer 2 | 902.4 902.99 902.99 |
| 710 | 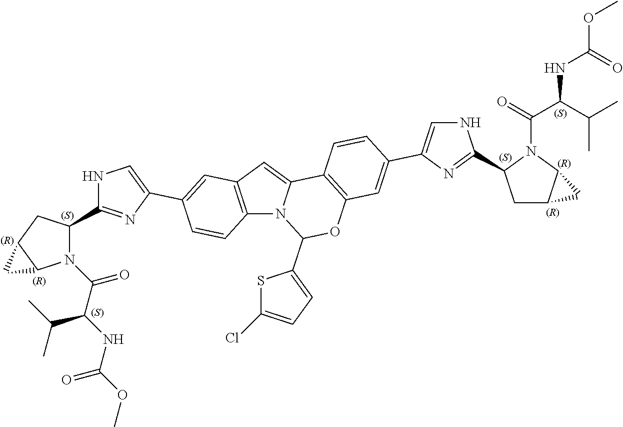 | racemic | 946.0 |
| 727 | 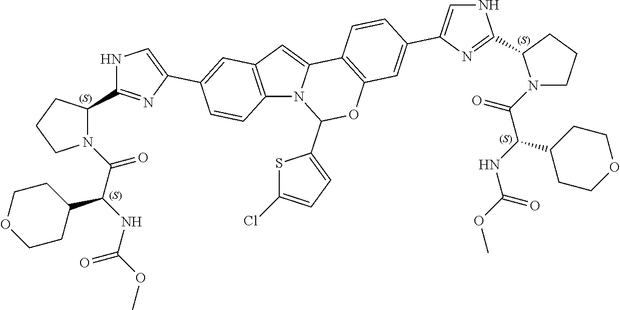 | racemic | 1006.0 |
| 728 | 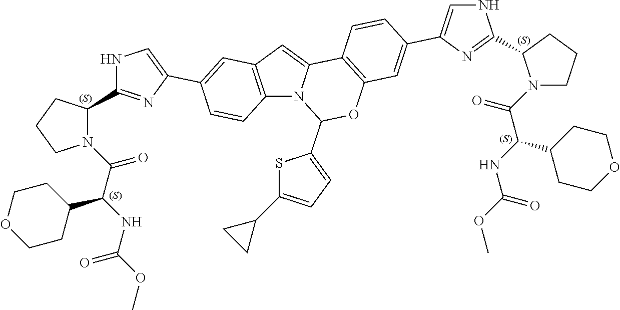 | racemic | 1012.4 |
| 732 | 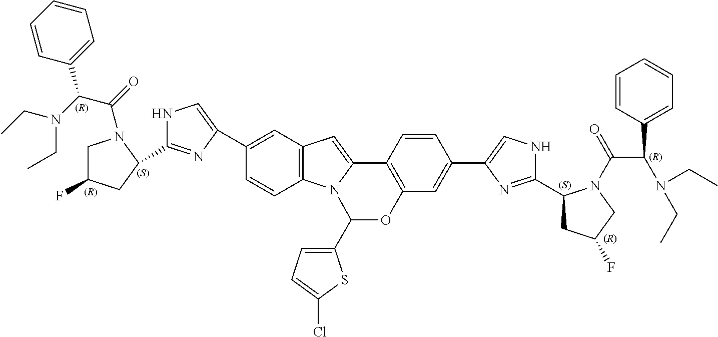 | racemic | 1022.3 |
| 733 | 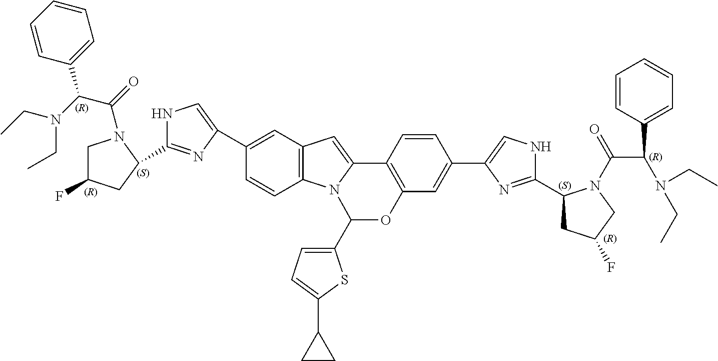 | racemic | 1028.0 |

### Example 81

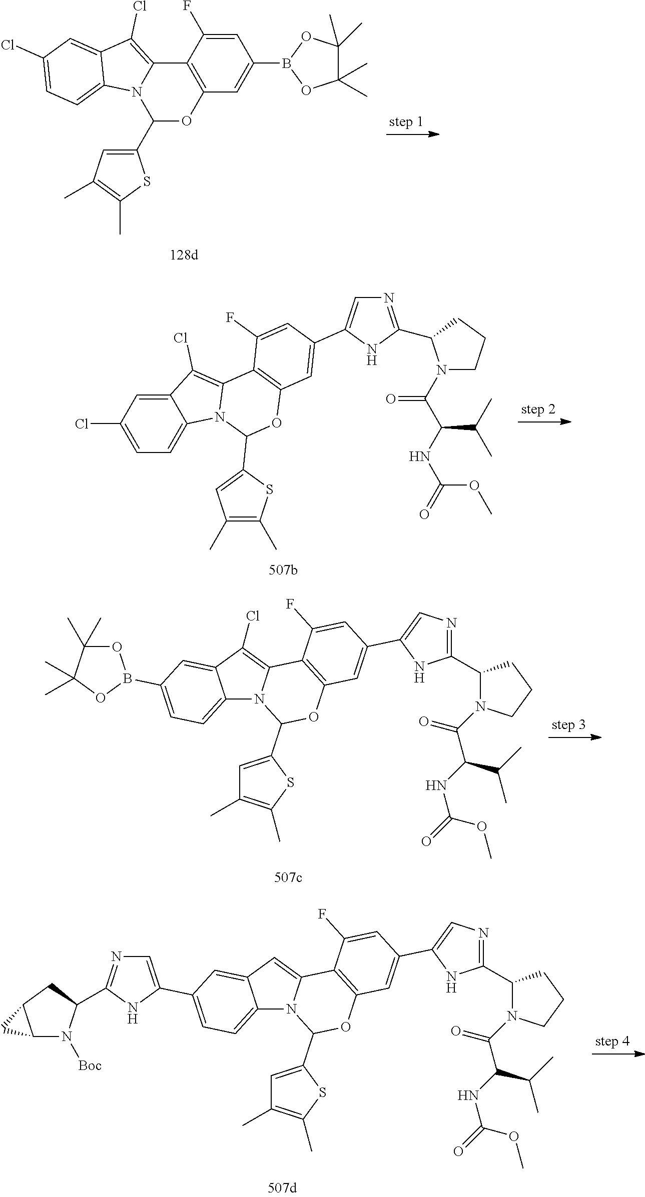

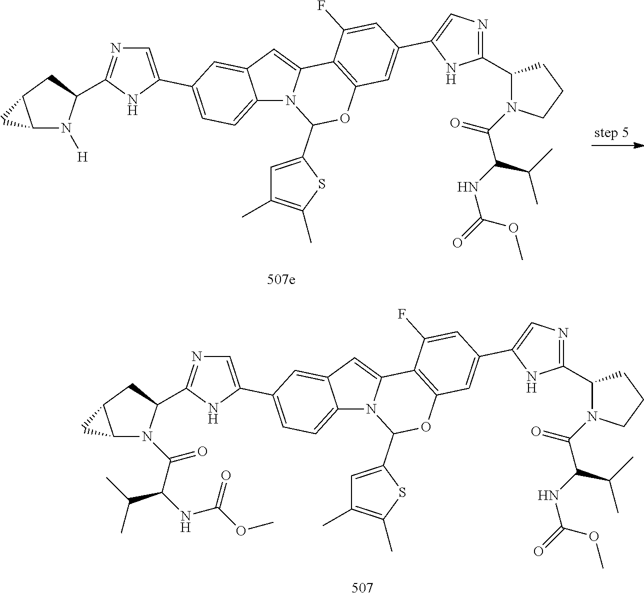

### Step 1

Compound 128d was prepared in Example 59. To the reaction mixture of 128d (0.679 g, 1.247 mmol) was added cap 31 (0.698 g, 1.871 mmol), KOAc (1N solution, 5 mL) and PdCl₂(dppf) (0.153 g, 0.187 mmol) and the tube was sealed and degassed and stirred at 100° C. for 16 hour. EtOAc (˜50 mL) was added and the mixture was washed with water, filtered through a celite pad. The filtrate was separated and the aqueous phase was extracted with EtOAc (2×20 mL). The combined organic fractions were washed with brine (20 mL), dried (Na₂SO₄), filtered and the solvent was evaporated under reduced pressure. The crude material was purified on a ISCO column (40 g) and eluted with Hex:EtOAc 0% to 90%. The product came out around 80% EtOAc to provide 507b (0.86 g, 92% yield).

### Step 2

The 507b (0.86 g, 1.21 mmol), bis(pinacolato)diboron (0.922 g, 3.63 mmol), KOAc (0.831 g, 8.47 mmol), Pd₂(dba)₃ (0.251 g, 0.242 mmol), and X-phos (0.173 g, 0.363 mmol) was added to a microwave tube and degassed three times before heated to 120° C. for 1 hour. LCMS of the crude reaction showed product, monodechloro product and bisdechloro product. The reaction was heated for another 10 hours. LCMS showed reaction done. The material was filtered through a celite pad, washing with EtOAc (˜20 mL). The solvent was evaporated under reduced pressure. The residue obtained was purified using column chromatography on silica gel (40 g column), eluting with 0-50%-80% EtOAc in Hex to provide 507c as a brown sticky gel (1.07 g, 70% yield, and 60% purity).

### Step 3

The cap 31a (0.25 g, 0.716 mmol), PdCl₂(dppf) (0.048 g, 0.065 mmol), and 1M K₂CO₃ (5 mL, 5 mmol) was added to dioxane (5 mL) solution of 507b (0.5 g, 0.651 mmol). The mixture was allowed to stir at 100° C. for 16 hours. LCMS showed reaction done. After cooling down, 30 mL EtOAc was added followed with ˜15 mL water, the aqueous layer was separated and extracted with EtOAc (15 mL×2, if layer separation was not clear, filtered the mixture through a celite pad). The organic layers were combined and dried over anhydrous Na₂SO₄. The solution was filtered and concentrated in vacuo. The residue obtained was purified using Flash column chromatography on silica gel (40 g, solvent A: DCM; solvent B: 10% MeOH to provide 507d as desired product (0.17 g, 30% yield).

### Step 4

To the DCM (˜2.5 mL) solution of 507d (0.17 g, 0.191 mmol) was added 1M HCl in dioxane (˜2.5 mL) and stirred at 20° C. for 1 hour, LCMS showed reaction done. The volatile was evaporated to provide 507e, which was used in the next step without purification. (0.14 g, 82% yield)

### Step 5

507e (0.08 g, 0.089 mmol), cap 31c (16.38 mg, 0.094 mmol), HATU (33.9 mg, 0.089 mmol), and DMF (2 mL) was added into a 40 mL flask. DIEA (47.6 μl, 0.267 mmol) was added. The solution was allowed to stir at 25° C. for 30 minutes. LCMS showed still some starting material, another 0.5 eq cap31c and HATU was added and stirred at 25° C. for another 20 min. Water (˜20 mL) was added. The solution was allowed to stir for 10 minutes, extracted with DCM (3×10 mL). The combined organic fractions were washed with brine (1×10 mL), dried (Na₂SO₄), filtered and the solvent was evaporated. The residue obtained was purified using Prep-TLC on silica gel, eluting with 0-50% then 70% 1% NH₃/10% MeOH in DCM to provide 507 as desired product (67.6 mg, 80% yield)

The following compounds of the present invention were made using the methods described in the Example above and substituting the appropriate reactants or reagents.

| | | | Observed |
|---|---|---|---|
| ID | Structure | Isomer | [M + H]+ |
| 501 502 503 | 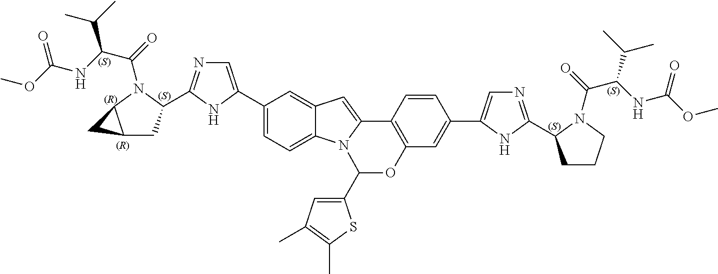 | isomer 1 isomer 2 racemic | 928.87 928.85 928.4 |
| 507 508 509 | 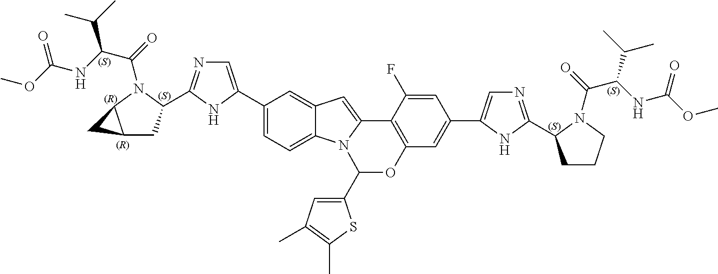 | Isomer 2 Racemic Isomer 1 | 946.96 946.78 946.95 |
| 514 515 516 | 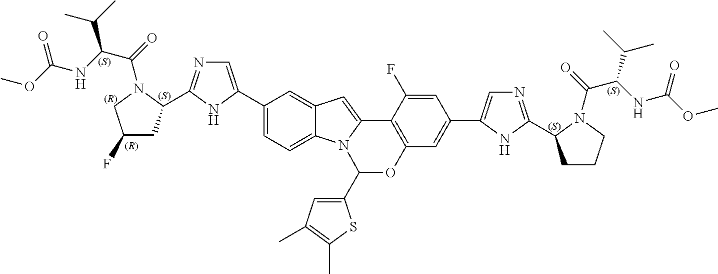 | Racemic Isomer 1 Isomer 2 | 953.15 952.96 952.82 |

### Example 82

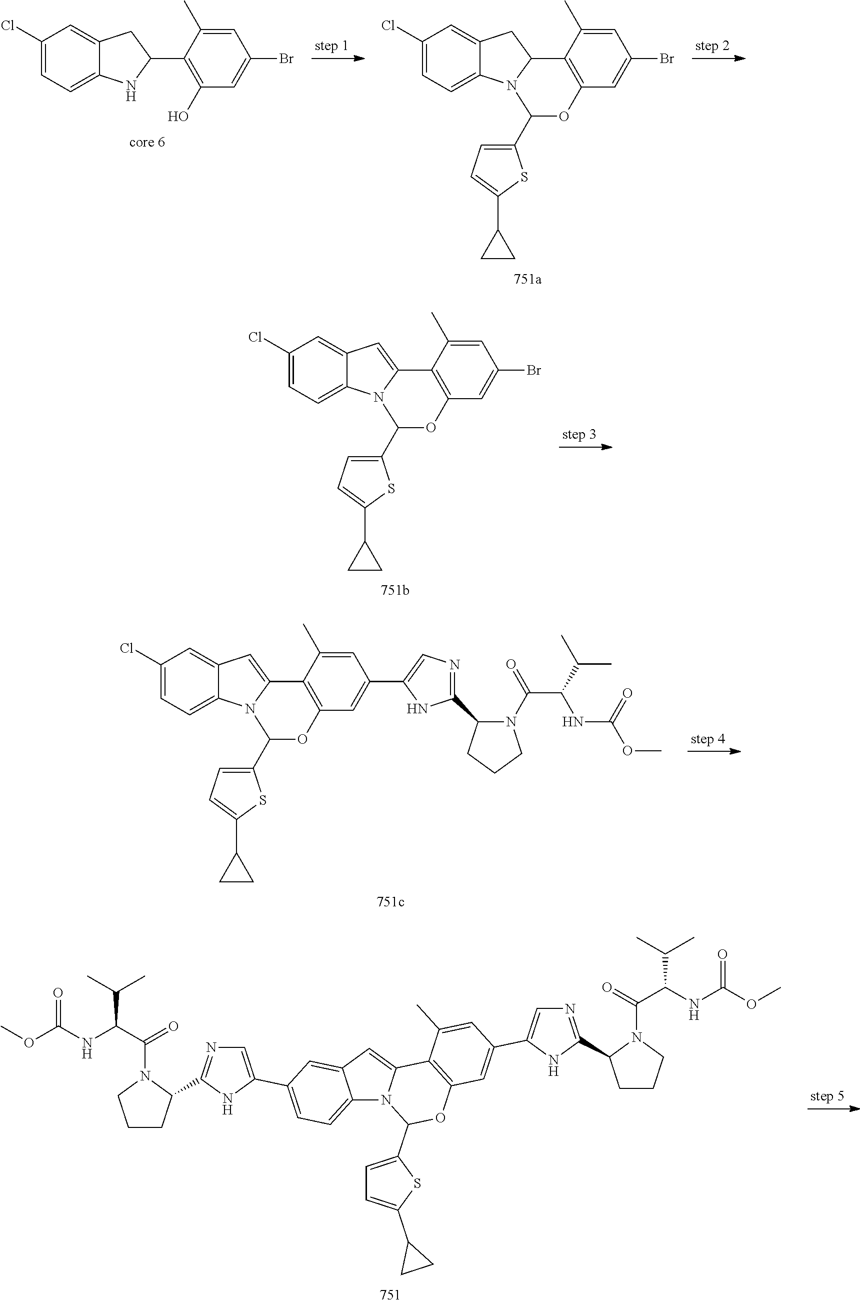

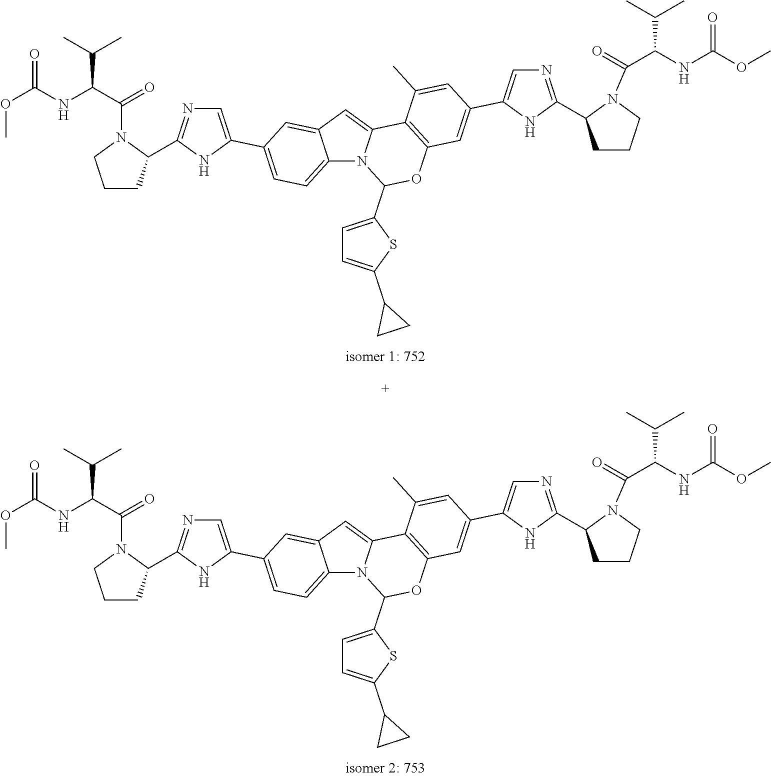 [SMILES: COC(=O)N[C@H](C(=O)N1CCC[C@H]1c1ncc(-c2cc(C)c3c(c2)OC(c2ccc(C4CC4)s2)n2c-3cc3cc(-c4cnc([C@@H]5CCCN5C(=O)[C@@H](NC(=O)OC)C(C)C)[nH]4)ccc32)[nH]1)C(C)C ; COC(=O)N[C@H](C(=O)N1CCC[C@H]1c1ncc(-c2cc(C)c3c(c2)OC(c2ccc(C4CC4)s2)n2c-3cc3cc(-c4cnc([C@@H]5CCCN5C(=O)[C@@H](NC(=O)OC)C(C)C)[nH]4)ccc32)[nH]1)C(C)C]

### Step 1

To a 10 mL flask was added core 6 (1.5 g, 4.43 mmol), 5-cyclopropylthiophene-2-carbaldehyde (0.742 g, 4.87 mmol), anhydrous acetonitrile (29.5 mL), and TFA (0.102 mL, 1.329 mmol). The solution was allowed to stir at 25° C. for 6 hours. The reaction mixture became a clear solution and then a solid appeared. The solid was collected by filtration, washed with acetonitrile, and dried in vacuo for about 15 hours to provide 751a (1.68 g, 80% yield).

### Step 2

To a 40 mL flask was added 751a (1.68 g, 3.55 mmol), toluene (23.7 mL), and DDQ (1.21 g, 5.33 mmol). The reaction was allowed to stir at 115° C. for 2 hours. EtOAc (100 mL) and sat. Na₂SO₃ (50 mL) was added. The organic layer was separated and washed with more Na₂SO₃ (50 mL), water, brine, dried over anhydrous Na₂SO₄, filtered and concentrated in vacuo. The product was purified using Flash column chromatography on silica gel (40 g, Hexane/EtOAc 0% to 30%) to provide 751b (1.51 g, 90% yield).

### Step 3

To a 40 mL flask was added 751b (1.5 g, 3.19 mmol), BIS(PINACOLATO)DIBORON (0.850 g, 3.35 mmol), PdCl2(dppf) (0.466 g, 0.637 mmol), and KOAc (0.938 g, 9.56 mmol). The flask was degassed by repeatedly been put on vacuum and re-filled with N₂. Dioxane (31.9 mL) was added and the reaction was allowed to stir at 90° C. for 2 hours. After cooling down, cap 31 (1.308 g, 3.50 mmol), PdCl₂(dppf) (0.233 g, 0.319 mmol), and K₂CO₃ (15.93 mL, 15.93 mmol) was added. The solution was allowed to stir at 80° C. for 8 hours. After cooling down, the aqueous layer was separated and extracted with EtOAc (20 mL). The organic layers were combined and concentrated in vacuo. The solution was concentrated in vacuo and purified using Flash column chromatography on silica gel (80 g, MeOH/DCM 0% to 10%, with addition of 1% concentrated NH₃.H₂O) to provide 751c (2.08 g, 95% yield). LC/MS: Anal. Calcd. For [M+H]⁺ C37H38ClN5O4S; 684.23. found 684.34.

### Step 4

To a 40 mL flask was added 751c (250 mg, 0.365 mmol), BisPINACOLATO)DIBORON (102 mg, 0.402 mmol), Pd₂(dba)₃ (33.5 mg, 0.037 mmol), X-PHOS (34.8 mg, 0.073 mmol), and KOAc (108 mg, 1.096 mmol). The flask was degassed by repeatedly been put on vacuum and re-filled with N₂. Dixane (3.54 mL) was added and the reaction was allowed to stir at 110° C. for 10 hours. After cooling down, methyl cap 31 (150 mg, 0.401 mmol), PdCl₂(dppf) (26.7 mg, 0.036 mmol), and K₂CO₃ (1M, 1.09 mL, 1.094 mmol) was added. The flask was capped and stirred at 80° C. for 10 hours. After cooling down, the aqueous layer was separated and extracted with EtOAc (10 mL). The organic layers were combined and concentrated in vacuo. The solution was concentrated in vacuo and purified using Flash column chromatography on silica gel (50 g, MeOH/DCM 0% to 10%, with addition of 1% concentrated NH₃—H₂O) to provide 751 (221 mg, 64.3% yield). LC/MS: Anal. Calcd. For [M+H]⁺ C51H59N9O7S; 942.43. found 942.79.

### Step 5

The two diastereo isomers of compound 751 (220 mg) were separated by SFC by using the following conditions.

Column: AS 20×250 mm

Solvent: 35% MeOH(0.2% DEA)/CO₂

Flow rate: 60 mL/min

Wavelength: 254 nm

752 (102 mg, 43% yield). LC/MS: Anal. Calcd. For [M+H]⁺ C51H59N9O7S; 942.43. found 943.15.

753 (98 mg, 41.3% yield). LC/MS: Anal. Calcd. For [M+H]⁺ C51H59N9O7S; 942.43. found 942.82.

The following compounds of the present invention were made using the methods described in the Example above and substituting the appropriate reactants or reagents.

| | | | Observed |
|---|---|---|---|
| ID | Structure | Isomer | [M + H]+ |
| 365 366 | 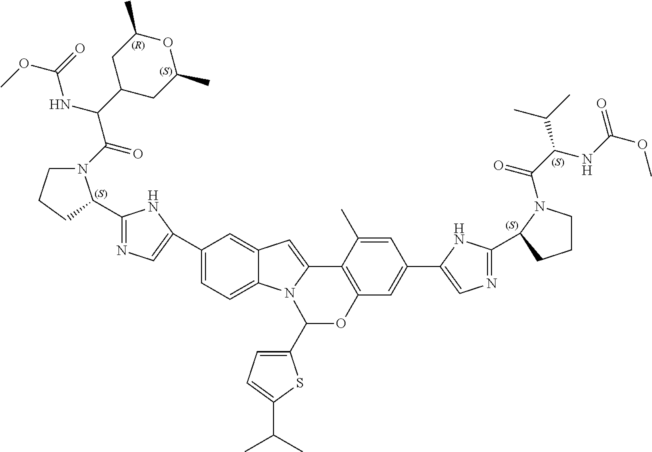 | isomer 1 isomer 2 | 1014.5 1014.5 |
| 751 752 753 | 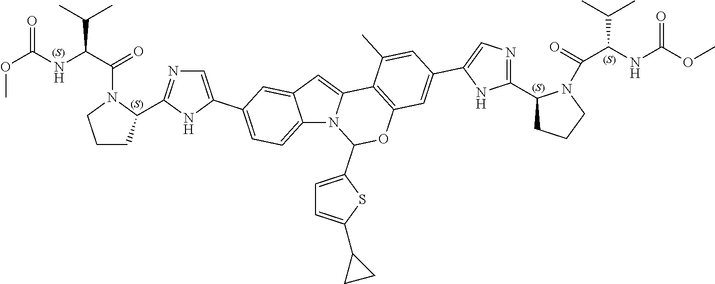 | racemic isomer 1 isomer 2 | 942.82 943.15 |
| 754 755 756 | 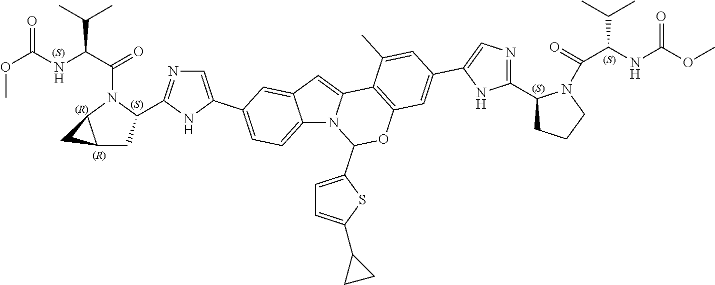 | racemic isomer 1 isomer 2 | 955.18 955.12 955.11 |
| 759 760 761 | 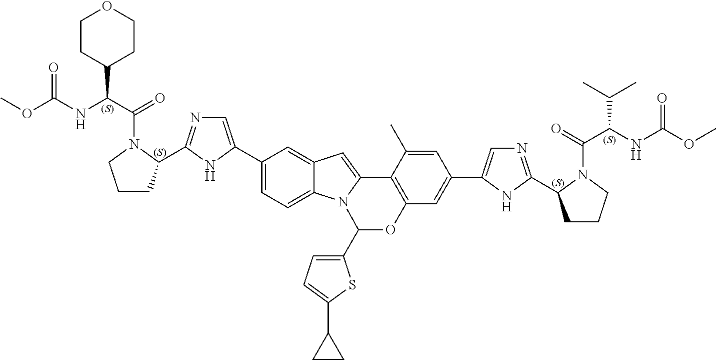 | racemic isomer 1 isomer 2 | 995.17 |
| 762 763 764 | 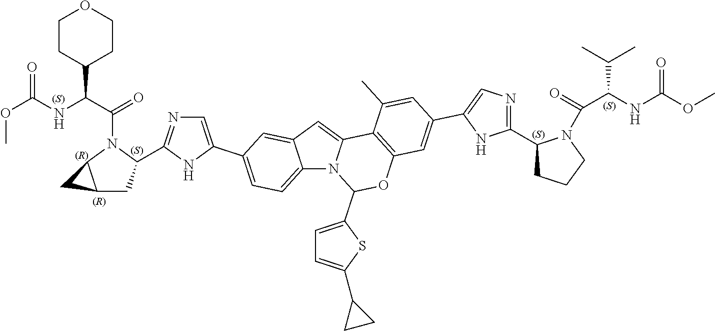 | racemic isomer 1 isomer 2 | 997.25 996.89 996.84 |
| 765 766 767 | 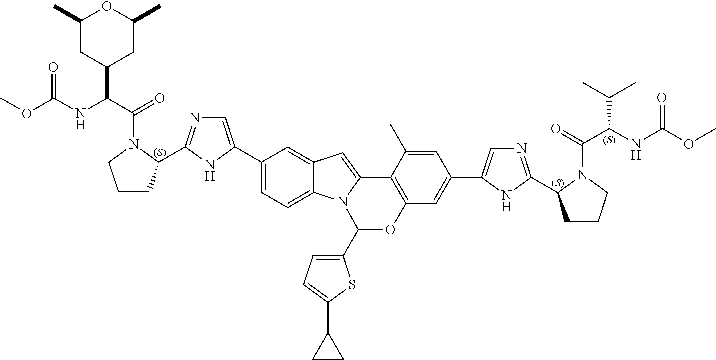 | racemic | 1013.23 |

### Example 83

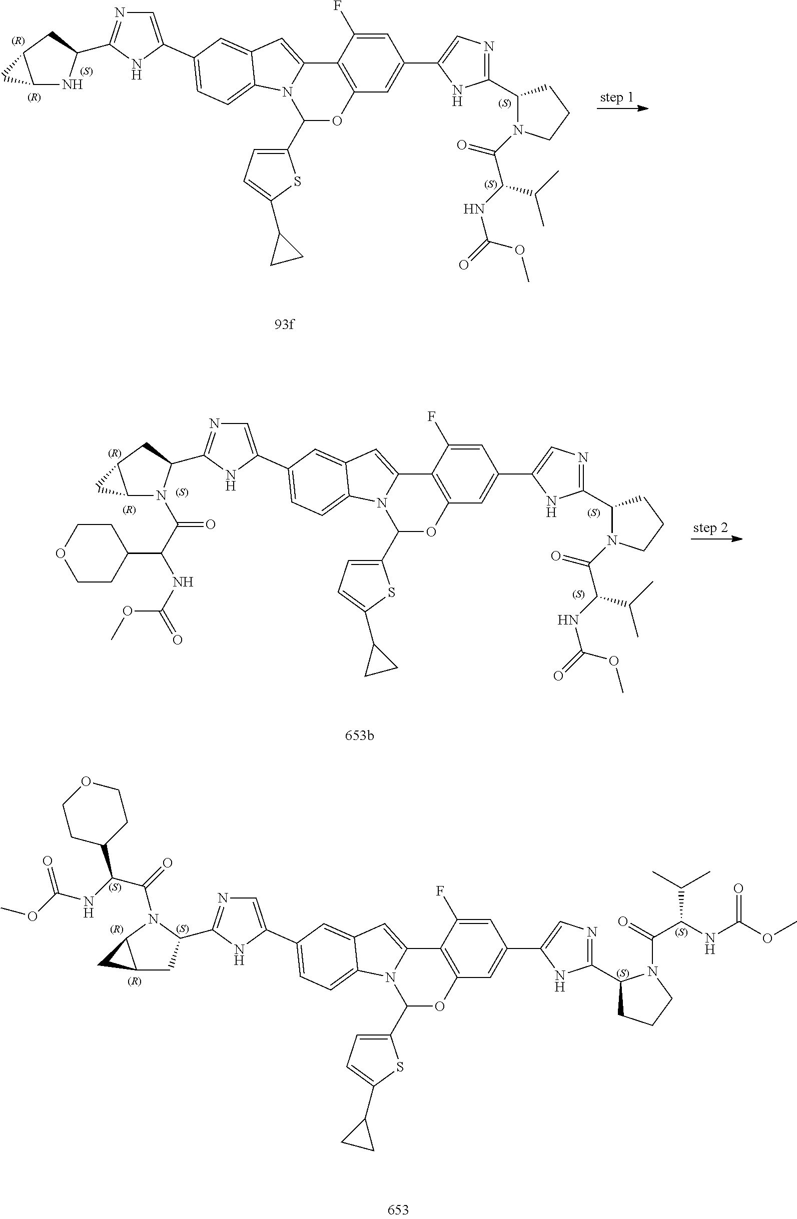

### Step 1

Compound 93f was prepared in Example 54. Compound 93f (200 mg, 0.220 mmol), cap 32c (47.7 mg, 0.220 mmol), HATU (84 mg, 0.220 mmol), and DMF (3 mL) was added into a 20 mL tube, cooled down to 0° C. by ice-water bath, DIPEA (0.196 mL, 1.099 mmol) was added. The solution was allowed to stir at 0° C. for 30 minutes. LCMS showed no starting material, water and EtOAc was added and organic layer was separated and washed with brine, dried under Na₂SO₄ anhydr., solvent was evaporated. Product 653b (90 mg, 0.085 mmol, 38.9% yield) was purified on 24g silica column 0% to 40% MeOH in CH₂Cl₂. LCMS: Anal. Calcd. For [M+H]⁺ C53H58FN9O8S; 1000.15. found 10001.31.

### Step 2

Compound 653 was separated from Compound 653b by the following

SFC conditions:

Column: AS-H,

Mobile phase: 40% EtOH+0.05% DEA

Compound 653 (Isomer B, 21.3 mg). LC/MS: Anal. Calcd. For [M+H]⁺ C53H58FN9O8S; 1000.15. found 10001.30.

The following compounds of the present invention were made using the methods described in the Example above and substituting the appropriate reactants or reagents.

| | | | Observed |
|---|---|---|---|
| ID | Structure | Isomer | [M + H]+ |
| 651 652 653 | 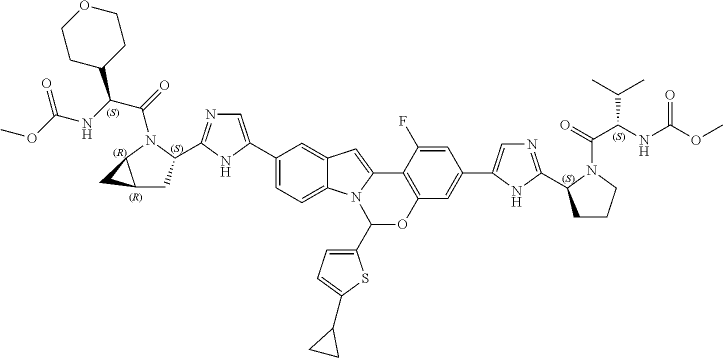 | racemic Isomer 1 Isomer 2 | 1001.31 1001.29 1001.30 |

### Example 84

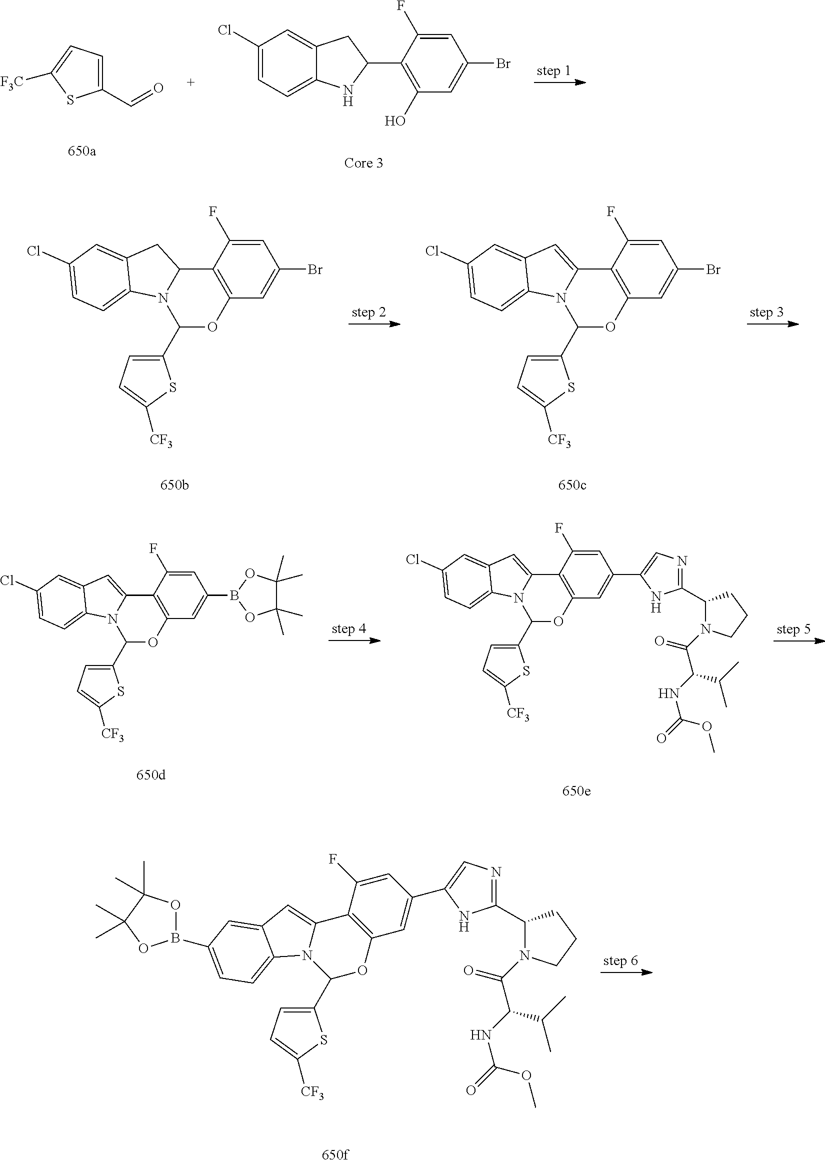

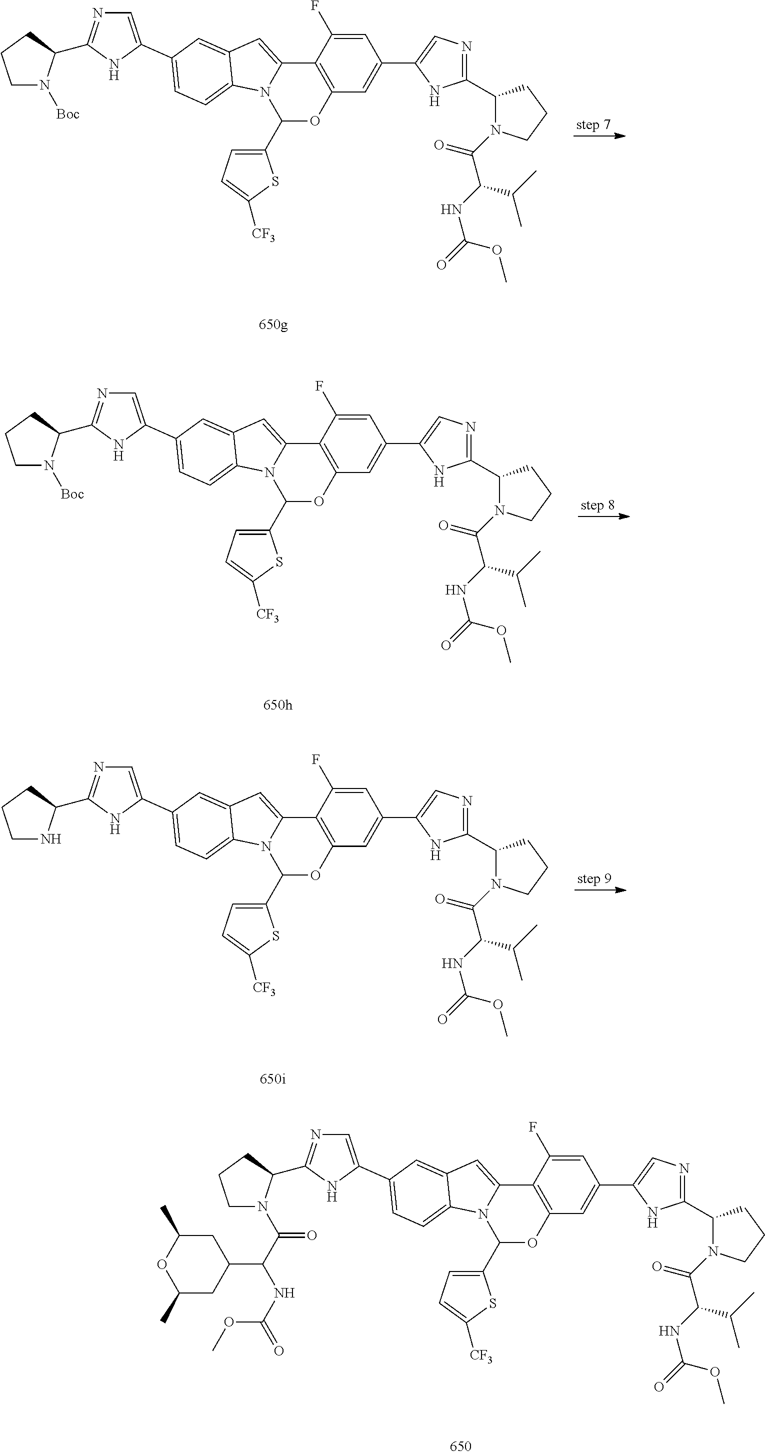

### Step 1

Core 3 (780 mg, 2.277 mmol) and 650a (410 mg, 2.277 mmol) were dissolved in ACN (5 mL) and then TFA (0.053 mL, 0.683 mmol) was added at 25° C. The mixture was allowed to stir for 4 hours. Precipitate was formed; TLC shows completion of the reaction. Precipitate was filtered and washed with MeOH to provide 650b (900 mg, 78% yield).

### Step 2

DDQ (607 mg, 2.67 mmol) was added to a stirred, mixture of 650b (900 mg, 1.783 mmol) in Toluene (20 mL) and the mixture was allowed to stir at 110° C. for 2 hours. The mixture was cooled, the solvent was removed in vacuo and the residue obtained was diluted with EtOAc (˜50 mL). The organic fractions were washed with sat. Na₂S₂O₃ and brine (saturated, 1×10 mL), dried under Na₂SO₄, filtered and the solvent was evaporated under reduced pressure. The residue obtained was washed with MeOH to provide 650c (720 mg, 80% yield). LC/MS: Anal. Calcd. For [M+H]⁺ C20H9BrClF4NOS; 500.92. found 502.95.

### Step 3

PdCl₂(dppf).CH₂Cl₂ (175 mg, 0.215 mmol) was added to the mixture of 650c (720 mg, 1.432 mmol), bis(pinacolato)diboron (436 mg, 1.719 mmol), potassium acetate (422 mg, 4.30 mmol) in Dioxane (10 mL) in pressure tube. The reaction mixture was purged with nitrogen, heated at 90° C. for 5 hours. Cooled to 25° C., and used as is 650d in the next step.

### Step 4

PdCl₂(dppf).CH₂Cl₂ (117 mg, 0.143 mmol) was added to a mixture of crude 650d, potassium carbonate (594 mg, 4.29 mmol) and cap 31 (534 mg, 1.432 mmol) in dioxane (10 mL) and Water (1 mL) in pressure tube. The reaction mixture was purged with nitrogen, heated at 90° C. for 2 hours. Cooled to 25° C., the reaction mixture was filtered, worked-up and solvent was evaporated. Crude was separated on flash LC (0%-100%)EtOAc/Hexane) to provide 650e (680 mg, 66.3% yield). LC/MS: Anal. Calcd. For [M+H]⁺ C34H30ClF4N5O4S; 715.16. found 716.52.

### Step 5

A mixture of 650e (680 mg, 0.950 mmol), bis(pinacolato)diboron (289 mg, 1.139 mmol), KOAc (280 mg, 2.85 mmol), Pd₂(dba)₃ (147 mg, 0.142 mmol), X-Phos (113 mg 0.237 mmol) in the dry dioxane 10 mL was degassed and sealed under N₂. The mixture was allowed to stir at 105° C. for 5 hours. The reaction mixture of the 650f was cooled and used as is in next step. LC/MS: Anal. Calcd. For [M+H]⁺ C40H42BF4N5O6S; 807.66. found 808.69.

### Step 6

A mixture of 650f from a previous step, cap 31a (339 mg, 1.071 mmol), K₂CO₃ (444 mg, 3.21 mmol) and Pd(dppf)Cl₂ (131 mg, 161 mmol) 10 mL Dioxane/1.5 ml H₂O in pressure tube was purged with nitrogen and vacuumed 2 times and stirred at 90° C. for 5 hours. After that, the mixture was washed with water and extracted with ethyl acetate, washed with brine and dried over anhydrous sodium sulfate. After being concentrated in vacuo, the residue obtained was purified using Flash column chromatography on silica gel, eluting with chromatography Ethyl acetate/Hexane (0% to 100%) to provide 650g (640 mg, 65.2% yield). LC/MS: Anal. Calcd. For [M+H]⁺ C46H48F4N806S; 916.68. found 918.05.

### Step 7

Compound 650h was separated from 650g (640 mg) by the following SFC conditions:

Column: OZ-H,

Mobile phase: 50% MeOH+0.05% DEA

Compound 650h (Isomer B, 120 mg). LC/MS: Anal. Calcd. For [M+H]⁺ C46H48F4N806S; 916.68. found 918.16.

### Step 8

To a solution of compound 650h (120 mg, 0.198 mmol) in dry dioxane (10 mL) was added HCl-dioxane 4M (0.327 mL) through syringe and stirred at 25° C. for 6 hours, then concentrated in vacuo and dried under high vacuum to provide the HCl salt of compound 650i (121 mg, 100%). LC/MS: Anal. Calcd. For [M+H]⁺ C41H40F4N804S; 816.28. found 818.03.

### Step 9

650i (60 mg, 0.065 mmol), cap 4 (15.89 mg, 0.065 mmol), HATU (24.63 mg, 0.065 mmol), and DMF (3 mL) was added into a 20 mL tube, cooled down to 0° C. by ice-water bath, DIPEA (0.058 mL, 0.324 mmol) was added. The solution was allowed to stir at 0° C. for 30 minutes. LC-MS showed no SM. The mixture was warmed up to 25° C., water and EtOAc was added and organic layer was separated and washed with brine, dried under Na₂SO₄, solvent was evaporated. Product 650 (27 mg, 39.1% yield) was purified on 24 g silica column 0% to 40% MeOH in CH₂Cl₂. LC/MS: Anal. Calcd. For [M+H]⁺ C52H57F4N9O8S; 1044.12. found 1046.54.

The following compounds of the present invention were made using the methods described in the Example above and substituting the appropriate reactants or reagents.

| | | | Observed |
|---|---|---|---|
| ID | Structure | Isomer | [M + H]+ |
| 649 650 | 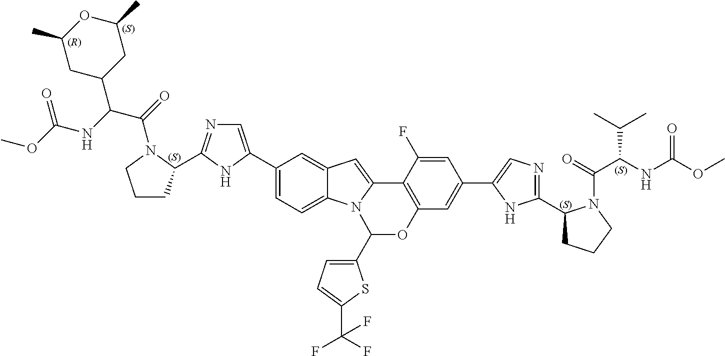 | Isomer 1 Isomer 2 | 1045.41 1046.54 |

### Example 85

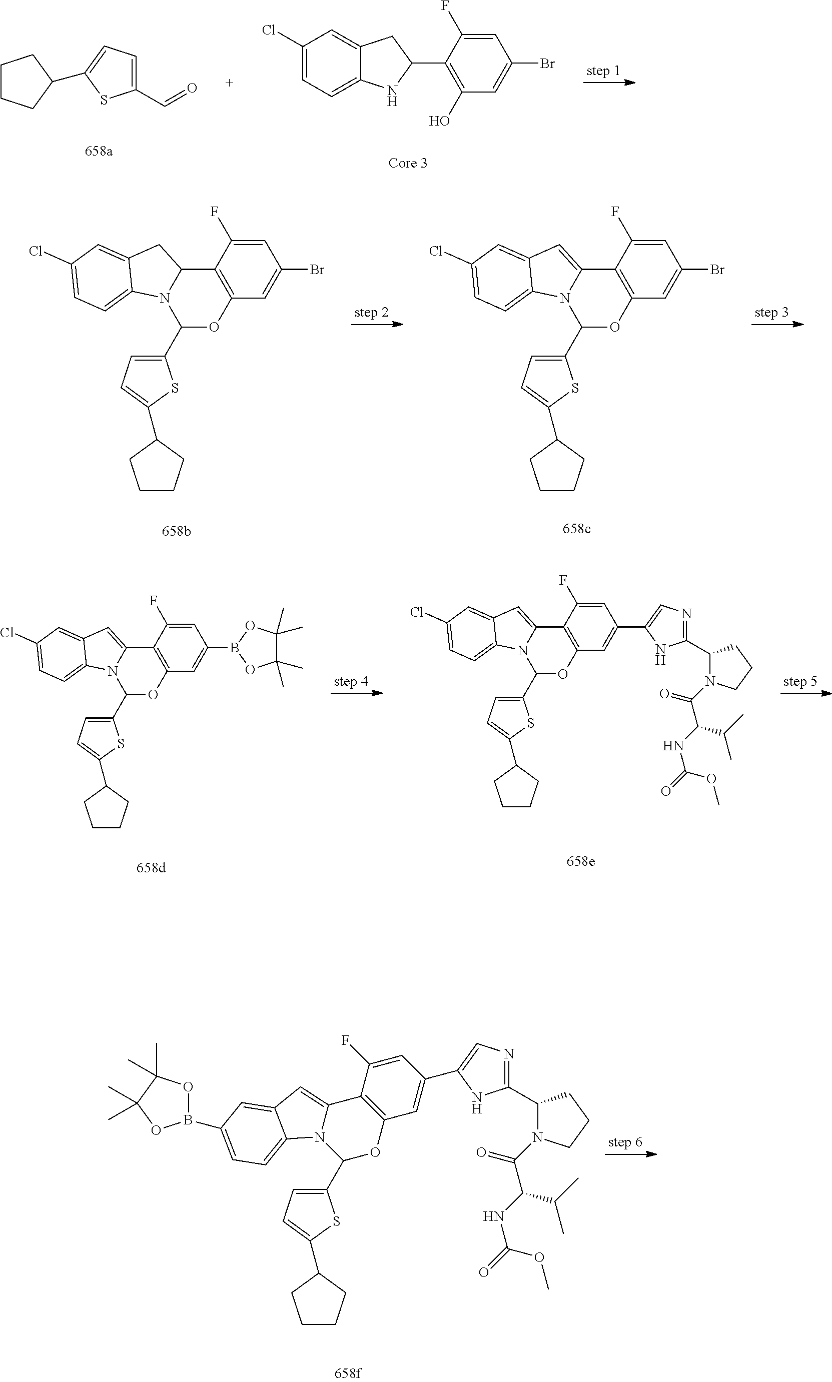

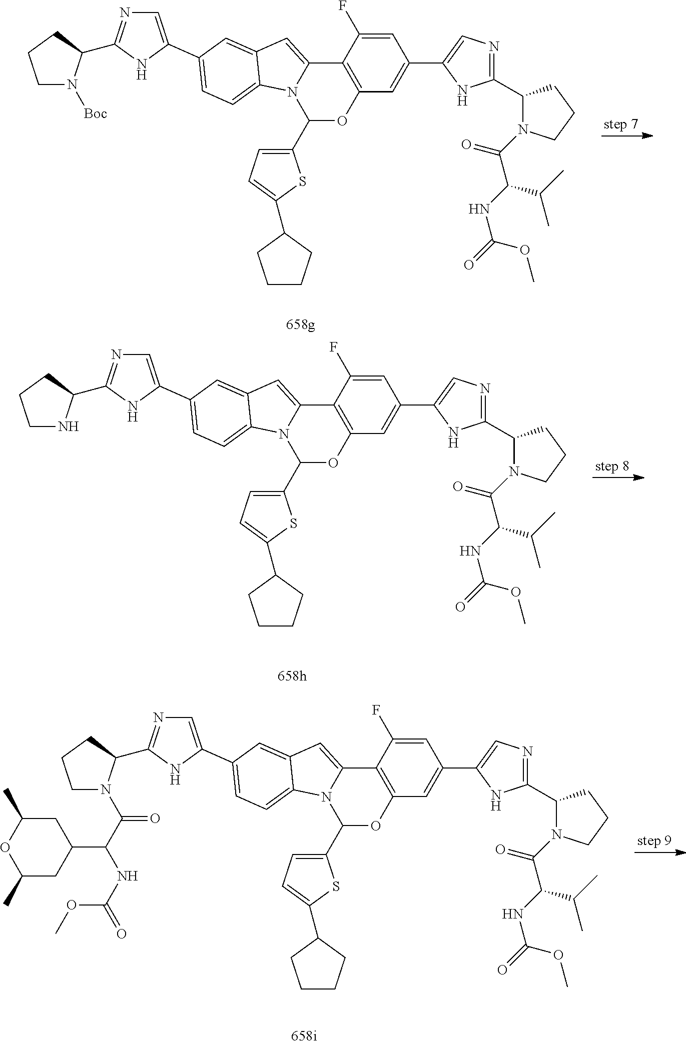 [SMILES: COC(=O)N[C@H](C(=O)N1CCC[C@H]1c1ncc(-c2cc(F)c3c(c2)OC(c2ccc(C4CCCC4)s2)n2c-3cc3cc(-c4cnc([C@@H]5CCCN5C(=O)OC(C)(C)C)[nH]4)ccc32)[nH]1)C(C)C ; COC(=O)N[C@H](C(=O)N1CCC[C@H]1c1ncc(-c2cc(F)c3c(c2)OC(c2ccc(C4CCCC4)s2)n2c-3cc3cc(-c4cnc([C@@H]5CCCN5)[nH]4)ccc32)[nH]1)C(C)C ; COC(=O)NC(C(=O)N1CCC[C@H]1c1ncc(-c2ccc3c(c2)cc2n3C(c3ccc(C4CCCC4)s3)Oc3cc(-c4cnc([C@@H]5CCCN5C(=O)[C@@H](NC(=O)OC)C(C)C)[nH]4)cc(F)c3-2)[nH]1)C1C[C@@H](C)O[C@@H](C)C1]

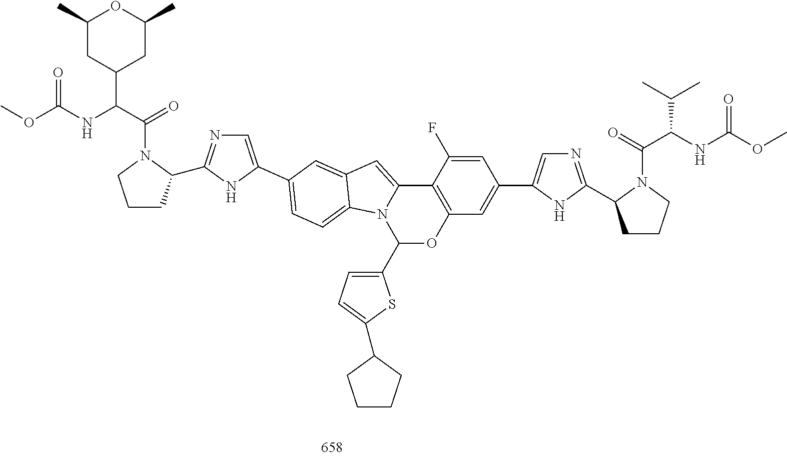

### Step 1

To a 20 mL tube was added core 3 (2.0 g, 5.84 mmol), 658a (1.158 g, 6.42 mmol), ACN (5 mL), and TFA (0.090 mL, 1.168 mmol) and the mixture was allowed to stir at 25° C. for 24 hours. Precipitant was formed. MeOH was added and solid filtered, and washed with MeOH to provide 658b (2.3 g, 78% yield).

### Step 2

DDQ (1.551 g, 6.83 mmol) was added to a stirred mixture of 658b (2.3 g, 4.56 mmol) in toluene (25 mL) and the mixture was allowed to stir at 110° C. for 2 hours. The mixture was cooled, the solvent was removed in vacuo and the residue obtained was diluted with EtOAc (˜50 mL). The organic fractions were washed with sat. Na₂S₂O₃, brine (saturated, 1×10 mL), dried under anh. Na₂SO₄, filtered and the solvent was evaporated under reduced pressure. The residue obtained was purified using column chromatography on silica gel 24 g, eluting with 0-10% EtOAc in Hexane to provide 658c (800 mg, 34.9% yield).

### Step 3

PdCl₂(dppf)-CH₂Cl₂Adduct (156 mg, 0.191 mmol) was added to a mixture of 658c (800 mg, 1.591 mmol), bis(pinacolato)diboron (444 mg, 1.750 mmol), KOAc (468 mg, 4.77 mmol) in dioxane (10 mL) in pressure tube. The reaction mixture was purged with nitrogen, heated at 90° C. for 5 hours. Cooled to 20° C., and used as is 658d in the next step.

### Step 4

PdCl₂(dppf)-CH₂Cl₂Adduct (195 mg, 0.239 mmol) was added to a mixture of the crude 658d, K₂CO₃ (660 mg, 4.77 mmol) and cap 31 (594 mg, 1.591 mmol) in dioxane (15 mL) and water (2 mL) in pressure tube. The reaction mixture was purged with nitrogen, heated at 90° C. for 5 hours. Cooled to 20° C., the reaction mixture was filtered, worked-up and solvent was evaporated. Crude was separated on flash LC (0%-100% EtOAc/Hexane) to provide 658e (450 mg, 39.1% yield). LC/MS: Anal. Calcd. For [M+H]⁺ C38H39ClFN5O4S; 716.26. found 716.87.

### Step 5

A mixture of 658e (450 mg, 0.622 mmol), bis(pinacolato)diboron (191 mg, 0.754 mmol), KOAc (185 mg, 1.885 mmol), Pd₂(dba)₃ (78 mg, 0.075 mmol), X-Phos (74.9 mg 0.157 mmol) in the dry dioxane 5 mL was degassed and sealed under N₂. The mixture was allowed to stir at 105° C. for 5 hours. The reaction mixture of the 658f was cooled and used as is in next step. LC/MS: Anal. Calcd. For [M+H]⁺ C44H51BFN5O6S; 807.78. found 809.09.

### Step 6

A mixture of 658f from a previous step, cap 31a (238 mg, 0.754 mmol), K₂CO₃ (261 mg, 1.885 mmol) and Pd(dppf)Cl₂ (61.6 mg, 0.075 mmol) in 5 mL dioxane/1 mL H₂O at pressure tube was purged with nitrogen and vacuumed 2 times and stirred at 90° C. for 5 hours. After that, the mixture was washed with water and extracted with ethyl acetate, washed with brine and dried over anhydrous sodium sulfate. After being concentrated in vacuo, the residue obtained was purified using Flash column chromatography on silica gel, eluting with chromatography Ethyl acetate/Hexane (0% to 100%) to provide 658g (308.4 mg, 53.5% yield). LC/MS: Anal. Calcd. For [M+H]⁺ C50H57FN8O6S; 917.10. found 918.24.

### Step 7

To a solution of 658g (165 mg, 0.180 mmol) in dry dioxane (10 mL) was added HCl-dioxane 4M (0.450 mL) through syringe and stirred at 25° C. for 6 hours, then concentrated in vacuo and dried under high vacuum to provide HCl salt of the desired product 658h (167 mg, 100% yield). LC/MS: Anal. Calcd. For [M+H]⁺ C45H49FN8O4S; 816.99. found 817.76.

### Step 8

658h (167 mg, 0.180 mmol), cap 4 (53.1 mg, 0.216 mmol), HATU (68.5 mg, 0.180 mmol), and DMF (3 mL) was added into a 20 mL tube, cooled down to 0° C. by ice-water bath, Diisopropylethylamine (0.16 mL, 0.901 mmol) was added. The solution was allowed to stir at 0° C. for 30 minutes. LC-MS showed no starting material, water and EtOAc was added and organic layer was separated and washed with brine, dried under Na₂SO₄, solvent was evaporated. 658i (89 mg, 47.3% yield) was purified on 24 g silica column 0% to 40% MeOH in CH₂Cl₂. LC/MS: Anal. Calcd. For [M+H]⁺ C56H66FN9O8S; 1044.24. found 1046.29.

### Step 9

Compound 658 was separated from 658i (89 mg) by the following SFC conditions:

Column: AS-H,

Mobile phase: 40% IPA+0.05% DEA

Compound 658 (Isomer B, 20 mg). LC/MS: Anal. Calcd. For [M+H]⁺ C56H66FN9O8S; 1044.24. found 1045.34.

The following compounds of the present invention were made using the methods described in the Example above and substituting the appropriate reactants or reagents.

| | | | Observed |
|---|---|---|---|
| ID | Structure | Isomer | [M + H]+ |
| 654 655 | 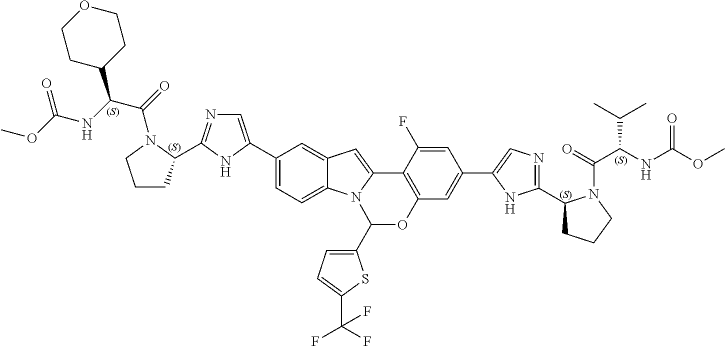 | Isomer 1 Isomer 2 | 1017.30 1017.20 |
| 656 657 | 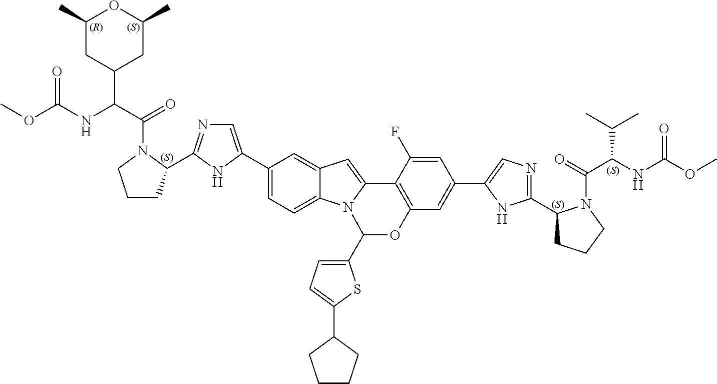 | Isomer 1 Isomer 2 | 1045.34 1045.34 |
| 540 | 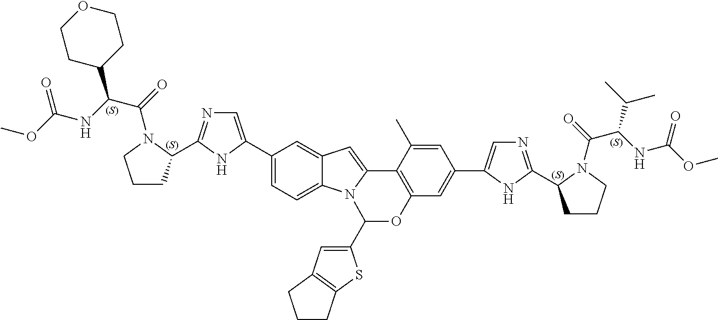 | Isomer 1 | 984.3 |
| 541 542 | 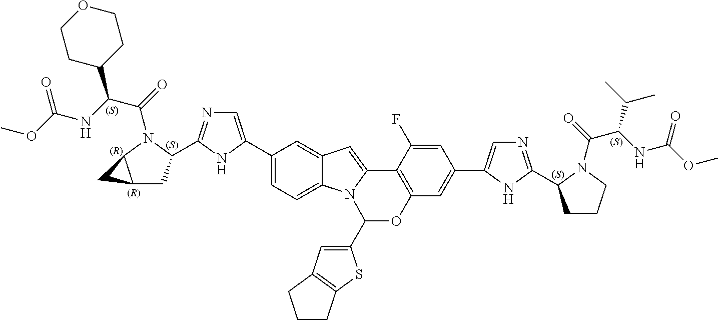 | Isomer 1 Isomer 2 | 1001.36 1001.36 |
| 548 549 550 | 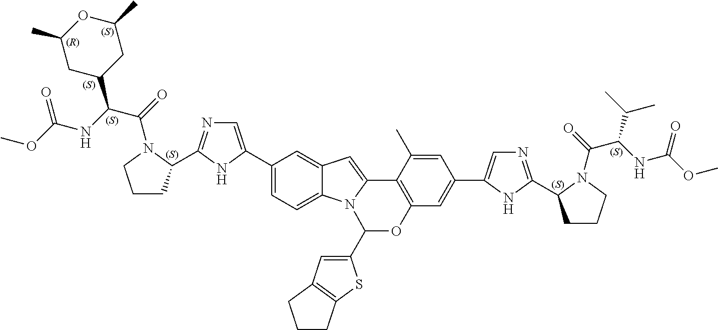 | Racemic Isomer 1 Isomer 2 | 1013.32 1013,3 1013.3 |
| 551 552 553 | 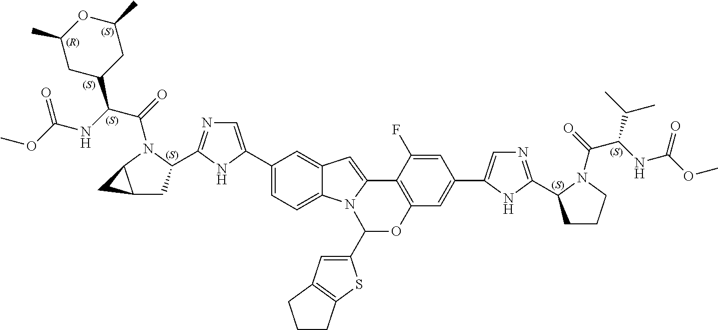 | Racemic Isomer 1 Isomer 2 | 1029.50 1029.50 1029.50 |

### Example 86

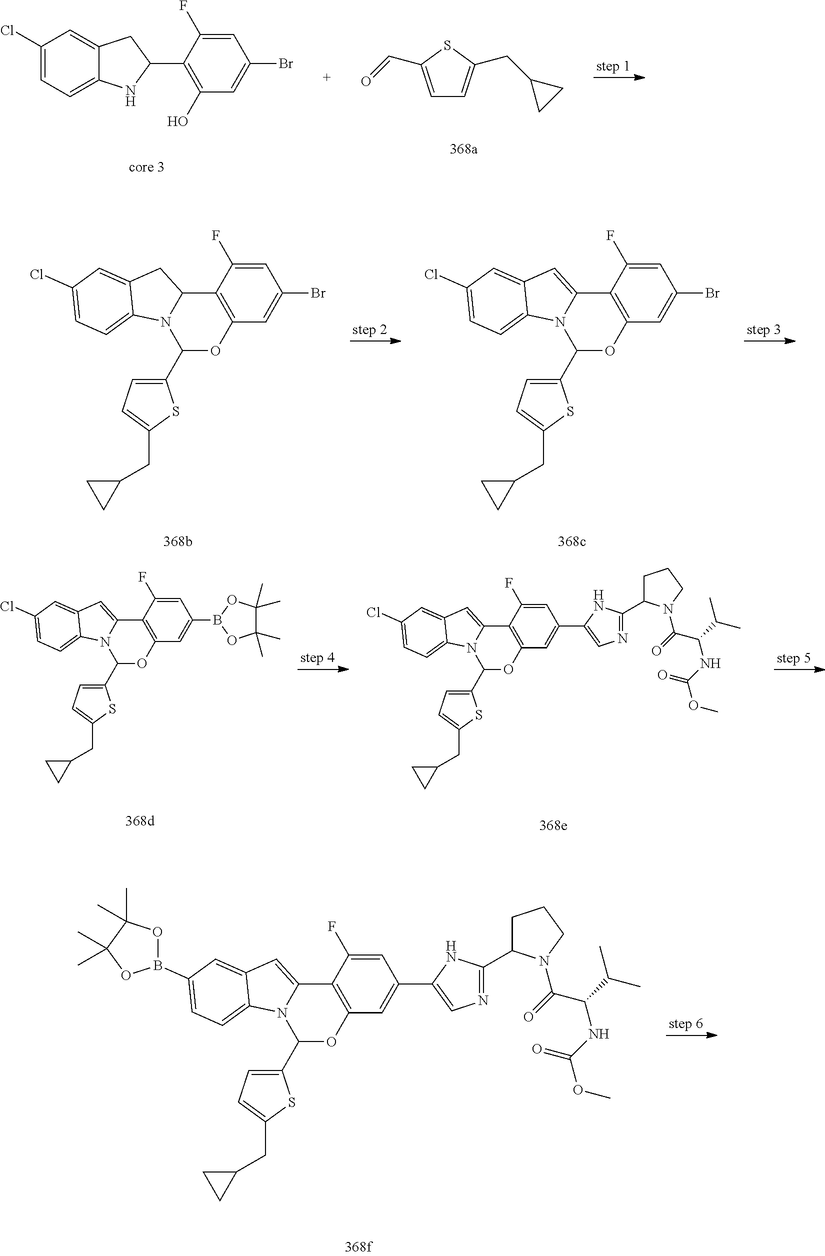

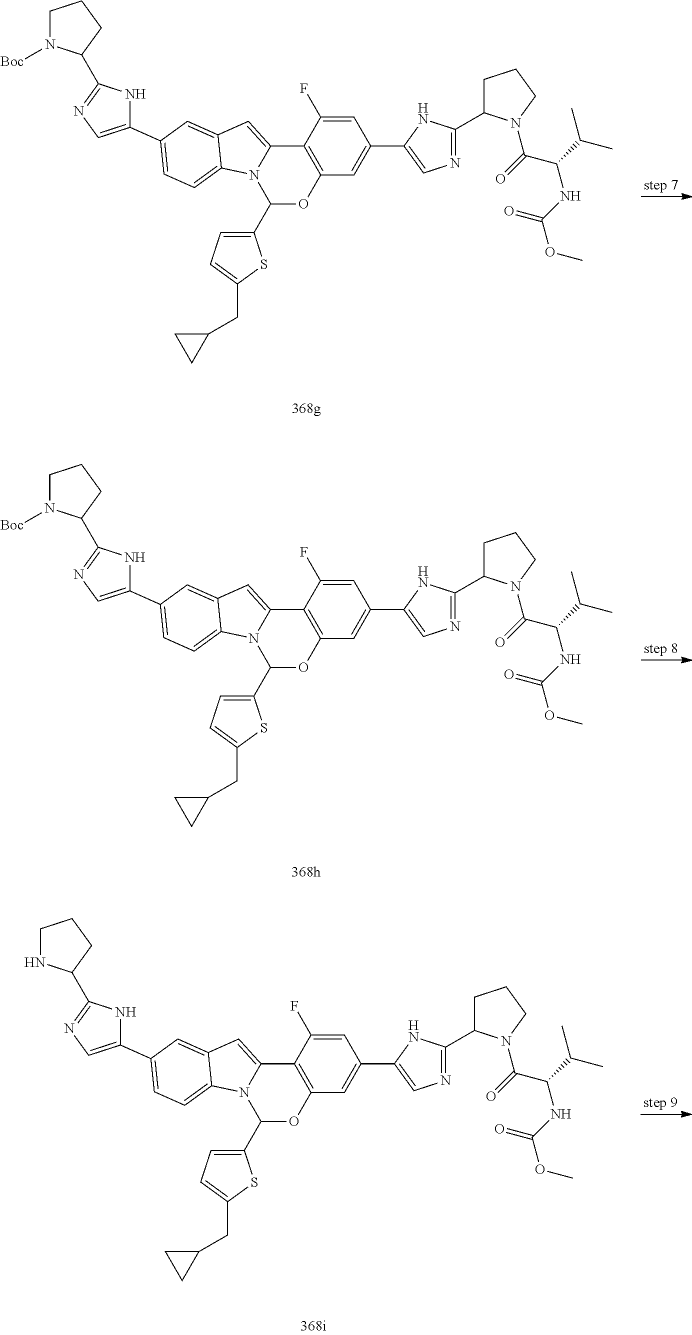 [SMILES: COC(=O)N[C@H](C(=O)N1CCCC1c1ncc(-c2cc(F)c3c(c2)OC(c2ccc(CC4CC4)s2)n2c-3cc3cc(-c4cnc(C5CCCN5C(=O)OC(C)(C)C)[nH]4)ccc32)[nH]1)C(C)C ; COC(=O)N[C@H](C(=O)N1CCCC1c1ncc(-c2cc(F)c3c(c2)OC(c2ccc(CC4CC4)s2)n2c-3cc3cc(-c4cnc(C5CCCN5C(=O)OC(C)(C)C)[nH]4)ccc32)[nH]1)C(C)C ; COC(=O)N[C@H](C(=O)N1CCCC1c1ncc(-c2cc(F)c3c(c2)OC(c2ccc(CC4CC4)s2)n2c-3cc3cc(-c4cnc(C5CCCN5)[nH]4)ccc32)[nH]1)C(C)C]

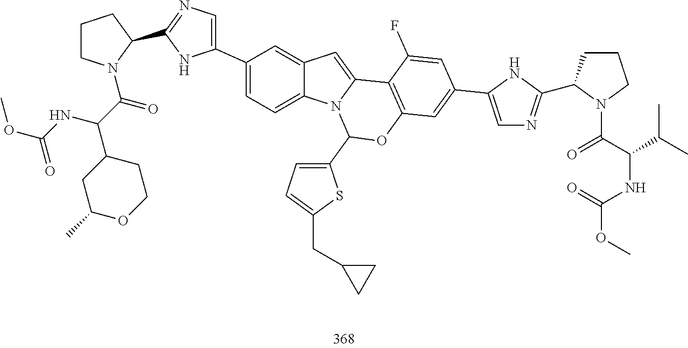 [SMILES: COC(=O)NC(C(=O)N1CCC[C@H]1c1ncc(-c2ccc3c(c2)cc2n3C(c3ccc(CC4CC4)s3)Oc3cc(-c4cnc([C@@H]5CCCN5C(=O)[C@@H](NC(=O)OC)C(C)C)[nH]4)cc(F)c3-2)[nH]1)C1CCO[C@H](C)C1]

### Step 1

Compound 368a was prepared in Example 43. To a mixture of core 3 (8.0 g, 23.1 mmol) and 368a (4 g, 24.0 mmol) in anhydrous CH₃CN (30 mL) was added TFA (1 mL). After the mixture was refluxed for 2 hours, the solvent was removed in vacuo and diluted with EtOAc. The organic layer was washed with saturated Na₂CO₃ aqueous and brine, dried over Na₂SO₄, filtered and concentrated in vacuo to provide 368b (8.3 g, 69% yield).

### Step 2

The solution of 368b (8.3 g, 15.6 mmol) in dry toluene (50 mL) was added DDQ (5.3 g, 23.3 mmol). After refluxing for 2 hours, the solvent was removed in vacuo and diluted with EtOAc. The organic layer was washed with saturated Na₂S₂O₃ aqueous and brine, dried over Na₂SO₄, filtered and concentrated in vacuo. The residue obtained was washed with MeOH (20 mL). The solid was collected to provide 368c (5.3 g, 64% yield).

### Step 3

A suspension of 368c (5.3 g, 11 mmol), bis(pinacolato)diboron (4.1 g, 16 mmol), KOAc (2.2 g, 22 mmol) and Pd(dppf)Cl₂ (800 mg, 1.1 mmol) in dioxane (25 mL) was allowed to stir at 100° C. for 2 hours under N₂ atmosphere. The reaction mixture was cooled and concentrated in vacuo, and the residue obtained was purified using Flash column chromatography on silica gel (80 g, EtOAc/Hexane 0% to 5%) to provide 368d (5.3 g, 90% yield).

### Step 4

A suspension of 368d (5.2 g, 9.7 mmol), cap 31 (5.4 g, 14.5 mmol), Na₂CO₃ (2 g, 19.4 mmol) and Pd(dppf)Cl₂ (732 mg, 1 mmol) in THF/H₂O (v/v=5/1, 120 mL) was allowed to stir at 80° C. for about 15 hours under N₂ atmosphere. After that, the mixture was washed with water and extracted with ethyl acetate, washed with brine and dried over anhydrous sodium sulfate. After being concentrated in vacuo, the residue obtained was purified using Flash column chromatography on silica gel (80 g, EtOAc/Hexane 10% to 50%) to provide 368e (5.3 g, 79% yield).

### Step 5

To a mixture of 368e (4.6 g, 6.6 mmol), bis(pinacolato)diboron (2.5 g, 9.8 mmol), KOAc (1.3 g, 13.2 mmol), Pd₂(dba)₃ (1.2 g, 1.3 mmol), X-Phos (1.2 g, 2.6 mmol) degassed and sealed under N₂ was added dry dioxane. Following further N₂ purging. The mixture was allowed to stir at 100° C. for about 15 hours. After cooling to room temperature, the solvent was concentrated in vacuo and the residue obtained was purified using Flash column chromatography on silica gel (30g, Hexane/EtOAc 20% to 50%) to provide 368f (4 g, 77% yield).

### Step 6

A mixture of 368f (4.6 g, 5.8 mmol), cap 32a (2.2 g, 7 mmol), Na₂CO₃ (1.3 g, 12 mol) and Pd(dppf)Cl₂ (440 mg, 0.6 mmol) in THF/H₂O (v/v=5/1, 50 mL) was allowed to stir at 80° C. under N₂ atmosphere for about 15 hours. After that, the mixture was washed with water and extracted with ethyl acetate, washed with brine and dried over anhydrous sodium sulfate. After being concentrated in vacuo, the residue obtained was purified using Flash column chromatography on silica gel (100 g, Hexane/EtOAc 30% to 200%) to provide 368g (3.6 g, 65% yield).

### Step 7

The compound of 368g (3.6 g) was separated by SFC by using the following conditions to provide 368h (1.3 g, 36%).

Instrument: Thar 80

Column: OZ 250 mm*20 mm, 5 um

Mobile phase: A for CO₂ and B for EtOH (0.05% NH3.H20)

Gradient: B 55% for A

Flow rate: 80 mL/min

Back pressure: 100 bar

Column temperature: 25° C.

Wavelength: 220 nm

### Step 8

To a solution of 368h (130 mg, 0.15 mmol) in 1,4-dioxane (15 mL) was added HCl/1,4-dioxane (15 mL, 3M). Then the mixture was allowed to stir at 20° C. for 2-3 hours. When the reaction completed, the mixture was concentrated in vacuo to provide 368i (120 mg, 100% yield).

### Step 9

To a mixture of 368i (120 mg, 0.15 mmol), cap 10 (37 mg, 0.15 mmol) and HATU (60 mg, 0.15 mmol) in DMF (3 mL) was added DIEA (80 mg, 0.6 mmol). The resulting mixture was allowed to stir at room temperature for 30 minutes, and LC-MS judged the material was consumed up. After filtrated, the filtrate was purified using Pre-HPLC to provide 368 (50 mg, 31% yield). ¹H NMR (MeOD) δ:7.88-7.93 (s, 1H), 7.73-7.78 (s, 1H), 7.68-7.72 (s, 1H), 7.61-7.66 (s, 1H), 7.33-7.45 (m, 2H), 7.21-7.26 (m, 1H), 7.16-7.21 (s, 1H), 7.02-7.08 (s, 1H), 6.45-6.52 (s, 1H), 6.36-6.41 (s, 1H), 4.98-5.15 (m, 2H), 4.02-4.18 (m, 2H), 3.89-4.01 (m, 2H), 3.65-3.82 (m, 2H), 3.52 (s, 6H), 2.33-2.51 (m, 4H), 1.78-2.18 (m, 8H), 1.38-1.49 (m, 2H), 1.11-1.25 (m, 3H), 0.89-1.01 (m, 3H), 0.68-0.87 (m, 6H), 0.26-0.38 (m, 2H), 0.01-0.05 (m, 2H)

The following compounds of the present invention were made using the methods described in the Example above and substituting the appropriate reactants or reagents.

| | | | Observed |
|---|---|---|---|
| ID | Structure | Isomer | [M + H]+ |
| 367 | 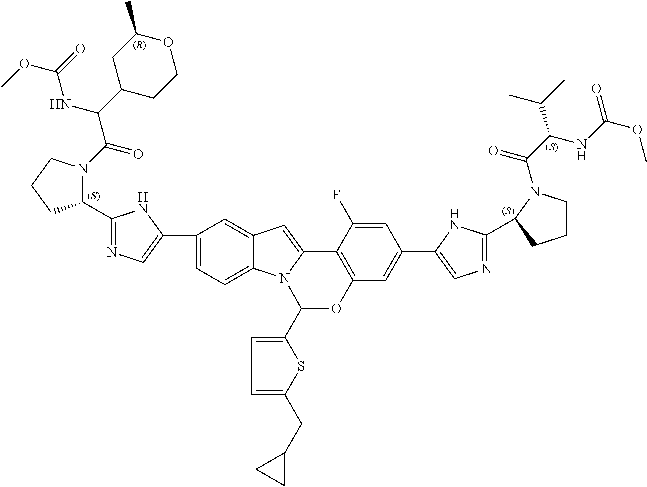 | Isomer 1 | 1016.5 |

### Example 87

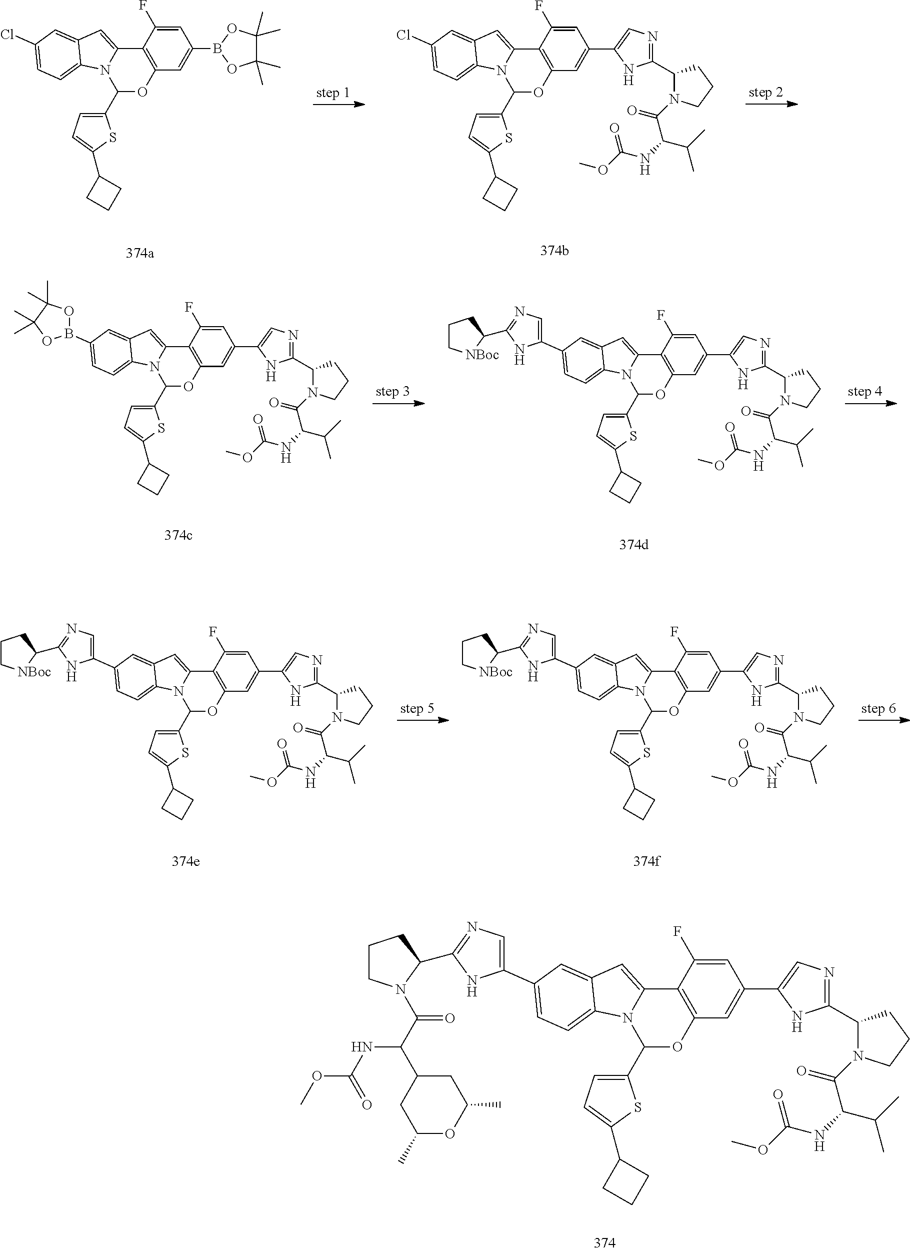

### Step 1

Compound 374a was prepared in Example 74. A suspension of 374a (2.3 g, 4.5 mmol), cap 31 (2.18 g, 5.85 mmol), Pd(dppf)Cl₂ (329 mg, 0.45 mmol) and Na₂CO₃ (1.5 g, 13.9 mmol) in THF/H₂O (5:1, 120 mL) was refluxed at 75° C. for about 15 hours under N₂ atmosphere. After that, the mixture was filtered; the filtrate was washed with water (50 mL) and extracted with EtOAc (100 mL), washed with brine and dried over anhydrous sodium sulfate. After being concentrated in vacuo, the residue obtained was purified using column chromatography (Petroleum Ether/EtOAc=5/1→1/1) to provide 374b (2.0 g, 63% yield).

### Step 2

To a mixture of 374b (2.2 g, 3.18 mmol), bis(pinacolato)diboron (0.95 g, 3.76 mmol), KOAc (0.91 g, 9.4 mmol), Pd₂(dba)₃ (286 mg, 0.21 mmol), X-Phos (147 mg, 0.31 mmol) degassed and sealed under N₂ was added dry dioxane. Following further N₂ purging. The mixture was allowed to stir at 120° C. for about 15 hours. Under standard work-up to provide the residue which was purified using column chromatography on silica to provide 374c (1.72 g, 69.3% yield).

### Step 3

A suspension of 374c (1.72 g, 2.18 mmol), cap 32a (759 g, 2.4 mmol), Pd(dppf)Cl₂ (160 mg, 0.22 mmol) and Na₂CO₃ (0.80 g, 7.4 mmol) in THF/H₂O (5:1, 24 mL) was refluxed at 75° C. for about 15 hours under N₂ atmosphere. After that, the mixture was filtered, the filtrate was washed with water (30 mL) and extracted with EtOAc (50 mL), washed with brine and dried over anhydrous sodium sulfate. After being concentrated in vacuo, the residue obtained was purified using column chromatography (Petroleum Ether/EtOAc=1/1→DCM/MeOH=3/1) to provide 374d (1.5 g, 76.1% yield).

### Step 4

Compound 374d (1.5 g) was separated by S.F.C. by using the following conditions.

Column: Chiral OZ 150×4.6 mm I.D., Sum

Mobile phase: 50% of methanol (0.05% DEA) in CO2

Flow rate: 2.0 mL/min

Wavelength: 340 nm

Compound 374e(650 mg, 43% yield).

### Step 5

Compound 374e (650 mg, 0.72 mmol) was added into HCl/dioxane (15 mL). Then the mixture was allowed to stir at 20° C. for 2-3 hours. When the reaction completed, the mixture was concentrated in vacuo to provide 374f (500 mg, 86.1% yield).

### Step 6

To a mixture of 374f (500 mg, 0.62 mmol), cap 4 (152 mg, 0.62 mmol) and HATU (238 mg, 0.62 mmol) in DMF (30 mL) was added DIPEA (241 mg, 1.867 mmol). The resulting mixture was allowed to stir at room temperature for 16 hours before the solution was subjected directly to HPLC to provide 374 (510 mg, 80% yield). ¹H NMR (MeOD) δ: 8.08-7.99 (m, 1H), 7.89 (s, 1H), 7.82 (s, 1H), 7.76 (s, 1H), 7.59-7.44 (m, 2H), 7.40-7.25 (m, 2H), 7.17 (br. s., 1H), 6.59-6.46 (m, 2H), 5.20 (td, J=7.4, 15.4 Hz, 2H), 4.28-4.02 (m, 4H), 3.85 (br. s., 2H), 3.64 (d, J=1.6 Hz, 5H), 3.57-3.38 (m, 3H), 2.63-2.45 (m, 2H), 2.32-2.08 (m, 8H), 2.06-1.85 (m, 5H), 1.76 (d, J=8.2 Hz, 1H), 1.55 (d, J=12.5 Hz, 1H), 1.34-1.24 (m, 1H), 1.19-1.03 (m, 6H), 1.02-0.78 (m, 9H) LC/MS: Anal. Calcd. For [M+H]⁺ C₅₅H₆₄FN₉O₈S; 1030.2. found 1030.

The following compound of the present invention was made using the methods described in the Example above and substituting the appropriate reactants or reagents.

| | | | Observed |
|---|---|---|---|
| ID | Structure | Isomer | [M + H]+ |
| 373 | 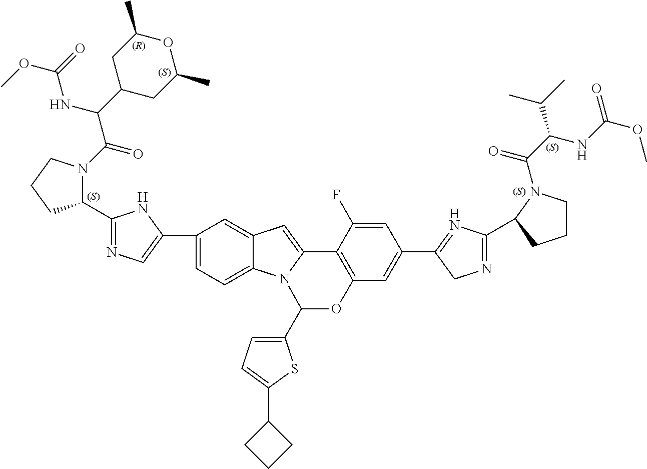 | Isomer 1 | 1030.5 |

### Example 88

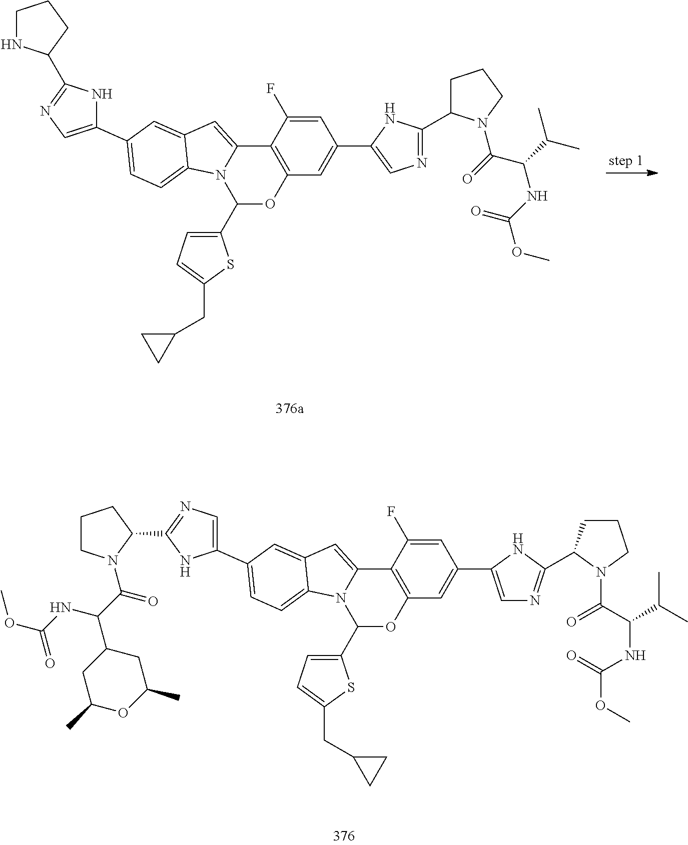

Compound 376a was prepared in Example 368. To a mixture of 376a (120 mg, 0.15 mmol), cap 4 (37 mg, 0.15 mmol) and HATU (60 mg, 0.15 mmol) in DMF (3 mL) was added DIEA (80 mg, 0.6 mmol). The resulting mixture was allowed to stir at 20° C. for 30 minutes, and LC-MS judged the material was consumed up. After filtrated, the filtrate was purified using Pre-HPLC to provide 376 (50 mg, 31% yield). ¹H NMR (MeOD) δ:7.87-7.95 (s, 1H), 7.76-7.83 (s, 1H), 7.69-7.74 (s, 1H), 7.63-7.68 (s, 1H), 7.39-7.45 (m, 1H), 7.33-7.38 (m, 1H), 7.22-7.28 (m, 1H), 7.18-7.22 (s, 1H), 7.03-7.10 (s, 1H), 6.46-6.52 (s, 1H), 6.35-6.43 (s, 1H), 5.02-5.20 (m, 2H), 4.05-4.20 (m, 2H), 3.91-4.03 (m, 2H), 3.67-3.82 (m, 2H), 3.54 (s, 6H), 3.23-3.40 (m, 2H), 2.35-2.56 (m, 4H), 1.82-2.19 (m, 8H), 1.36-1.49 (m, 1H), 1.14-1.24 (m, 1H), 0.92-1.09 (m, 6H), 0.68-0.89 (m, 6H), 0.29-0.39 (m, 2H), 0.06-0.08 (m, 2H).

The following compound of the present invention was made using the methods described in the Example above and substituting the appropriate reactants or reagents.

| | | | Observed |
|---|---|---|---|
| ID | Structure | Isomer | [M + H]+ |
| 375 | 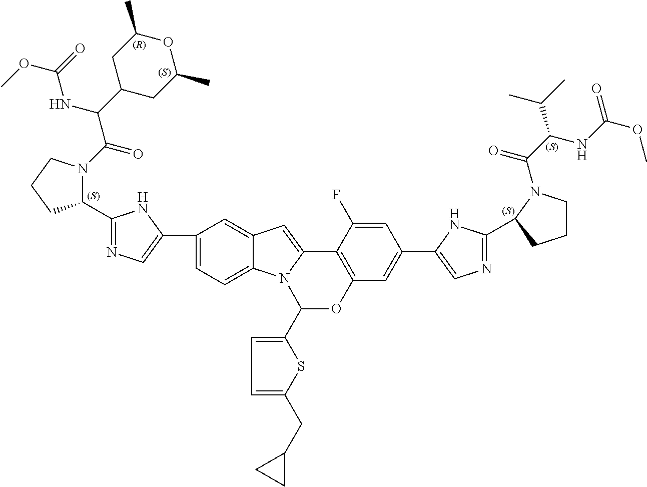 | Isomer 1 | 1030.5 |

### Example 89

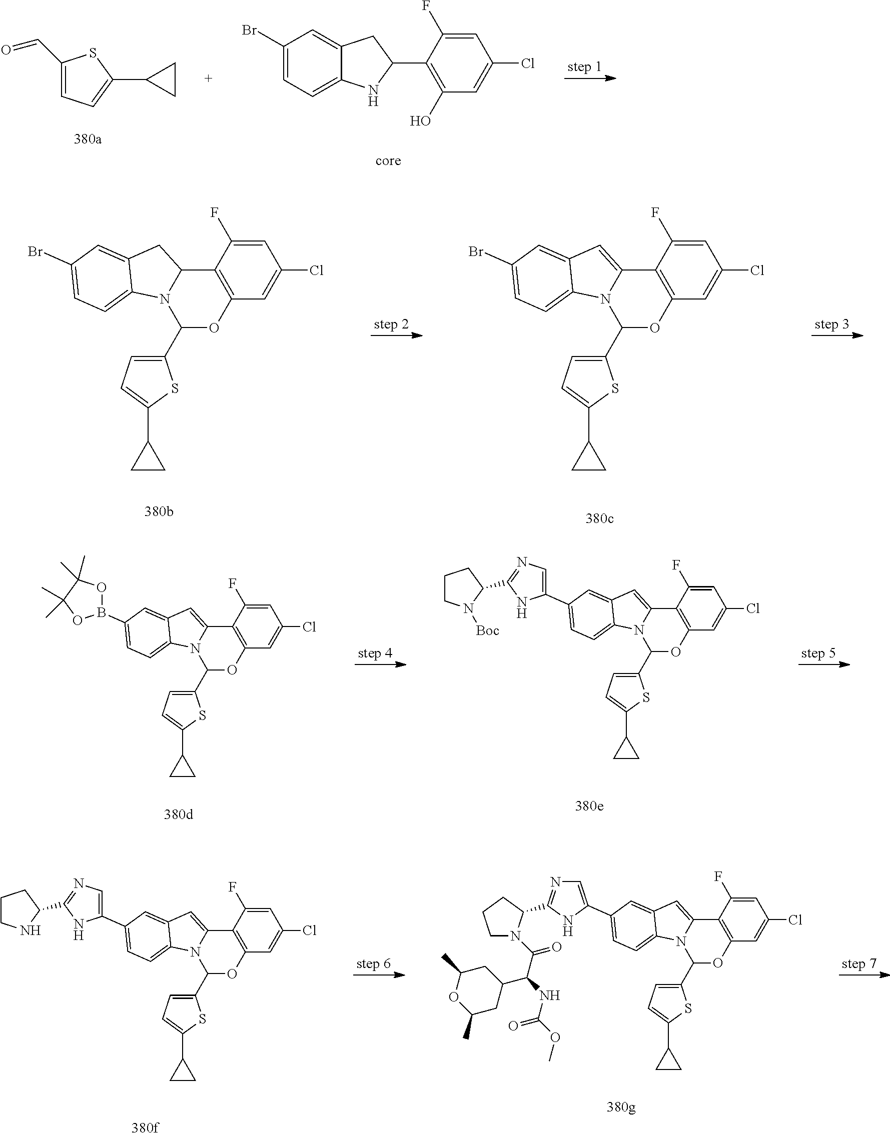

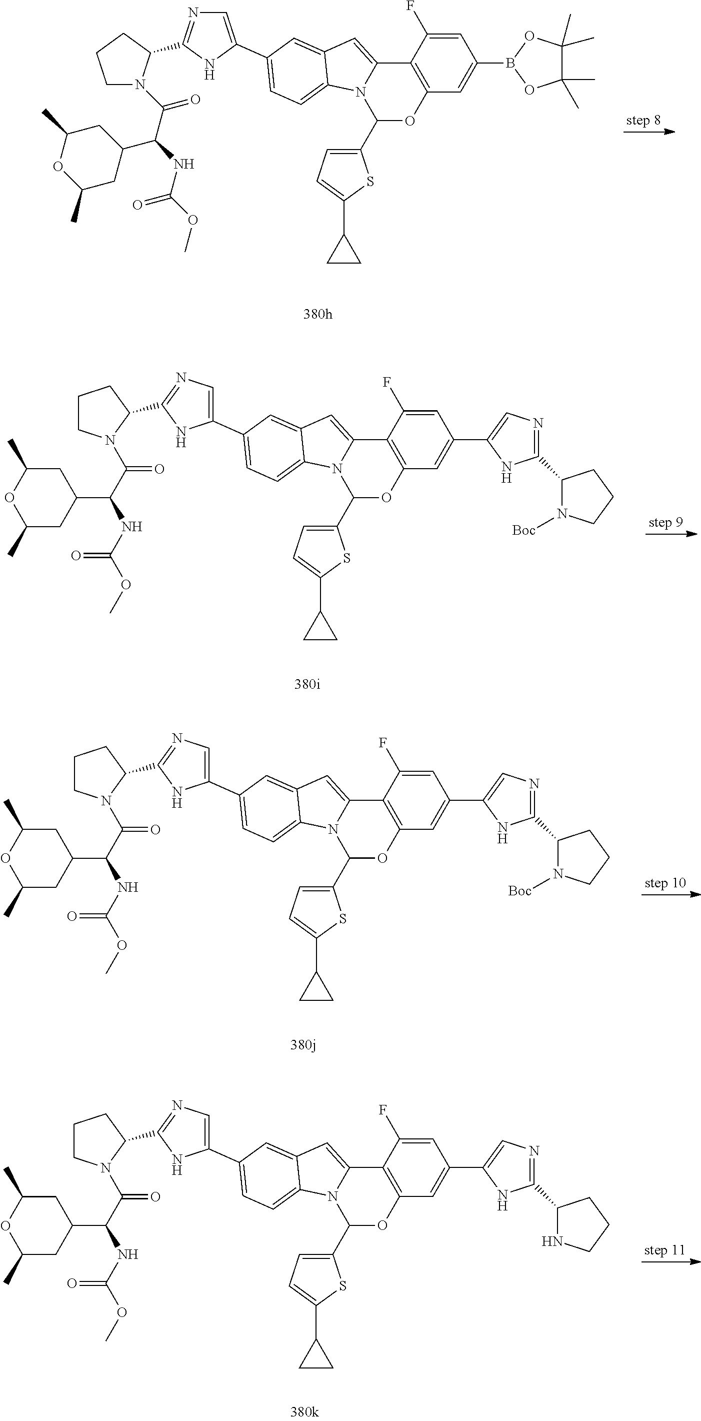

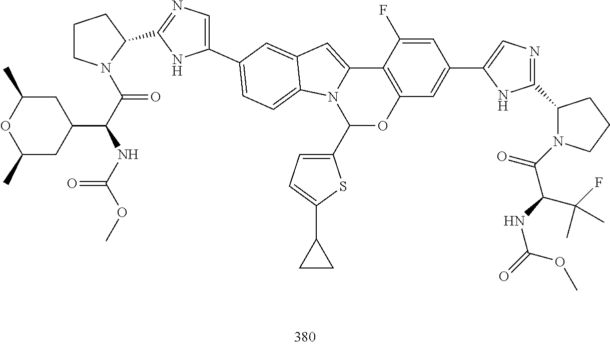 [SMILES: COC(=O)N[C@H](C(=O)N1CCC[C@@H]1c1ncc(-c2ccc3c(c2)cc2n3C(c3ccc(C4CC4)s3)Oc3cc(-c4cnc([C@@H]5CCCN5C(=O)[C@H](NC(=O)OC)C(C)(C)F)[nH]4)cc(F)c3-2)[nH]1)C1C[C@@H](C)O[C@@H](C)C1]

### Step 1

Compound 380a was prepared in Example 33. To a mixture of 380a (8.8 g, 58 mmol) and core 4 (10 g, 29 mmol) in anhydrous CH₃CN (100 mL) was added TFA (0.1 mmol) at 25° C. The mixture was agitated for 6 hours at 25° C. The reaction mixture became a clear solution and then a solid appeared. The solid 380b was collected by filtration and washed with CH₃CN (11 g, 79% yield). LC/MS: Anal. Calcd. For [M+H]⁺ C22H16BrClFNOS; 476.98. found 478.

### Step 2

The solution of 380b (11 g, 23 mmol) in dry toluene (200 mL) was added DDQ (7.8 g, 35 mmol). After refluxing for 2 hours, the solvent was removed in vacuo and diluted with hexyl acetate. The organic layer was washed with saturated Na₂S₂O₃ aqueous and brine, dried over Na₂SO₄, filtered and concentrated in vacuo. The residue obtained was washed with methanol (10 mL), filtered and the solid just was the compound 380c (8.7 g, 79.8% yield). LC/MS: Anal. Calcd. For [M+H]⁺ C22H14BrClFNOS; 474.96. found 477.

### Step 3

To a suspension of 380c (8.7 g, 18.3 mmol), bis(pinacolato)diboron (5.6 g, 22 mmol), KOAc (5.4 g, 54.9 mmol) and Pd(dppf)Cl₂ (1.33 g, 1.8 mmol) in dioxane (200 mL) was allowed to stir at 100° C. under N₂ atmosphere for about 15 hours. The reaction mixture was cooled and concentrated in vacuo, then chromatographed on silica gel (Petroleum Ether:EtOAc=10:1) gave 380d (8.8 g, 91.6% yield). LC/MS: Anal. Calcd. For [M+H]⁺ C28H26BClFNO3S; 521.14. found 522.

### Step 4

A suspension of 380d (8.8 g, 17 mmol), cap 32a (6.3 g, 20 mmol), Pd(dppf) Cl₂ (1.24 g, 1.7 mmol), Na₂CO₃ (5.4 g, 51 mmol) and in THF/H₂O (5:1, 192 mL) was refluxed at 80° C. for about 15 hours under N₂ atmosphere. After that, the mixture was filtered, the filtrate was washed with water (50 mL) and extracted with EtOAc (100 mL), washed with brine and dried over anhydrous sodium sulfate. After being concentrated in vacuo, the residue obtained was purified using column chromatography (Petroleum Ether:EtOAc=1:1) to provide compound 380e (8 g, 75% yield). LC/MS: Anal. Calcd. For [M+H]⁺ C34H32ClFN4O3S; 630.19. found 631.

### Step 5

Compound 380e (8 g, 12.7 mmol) was added into HCl/dioxane (40 mL). Then the mixture was allowed to stir at 20° C. for 2-3 hours. When the reaction completed, the mixture was concentrated in vacuo to provide the crude 380f (6.6 g, 82.7% yield).

### Step 6

To a mixture of 380f (6.6 g, 12.4 mmol), cap 4 (3.1 g, 12.4 mmol) and DIPEA (1 mL) in DMF (100 mL) was added HATU (4.8 g, 12 mmol). The resulting mixture was allowed to stir at 20° C. The mixture was purified using Pre-HPLC to provide compound 380g (6.5 g, 74.4% yield). LC/MS: Anal. Calcd. For [M+H]⁺ C40H41ClFN5O5S; 757.25. found 757.1.

### Step 7

To a mixture of 380g (6.5 g, 8.6 mmol), bis(pinacolato) diboron (0.76 g, 3 mmol), KOAc (2.5 g, 25.8 mmol), Pd₂(dba)₃ (0.8 g, 0.86 mmol), X-Phos (0.4 g, 0.86 mmol) degassed and sealed under N₂ was added dry dioxane. Following further N₂ purging. The mixture was allowed to stir at 100° C. for about 15 hours for about 15 hours. Under standard work-up to provide the residue which was purified using column chromatography on silica (Petroleum Ether:EtOAc=1:2) to provide product 380h (7.5 g, 98.6% yield). LC/MS: Anal. Calcd. For [M+H]⁺ C46H53BFN5O7S; 849.37. found 850.

### Step 8

A suspension of 380h (7.5 g, 8.8 mmol), cap 32a (3.35 g, 11 mmol), Pd(dppf) Cl₂ (0.64 g, 0.88 mmol), Na₂CO₃ (3.37 g, 31.8 mmol) and in THF/H₂O (5:1, 180 mL) was refluxed at 80° C. for about 15 hours under N₂ atmosphere. After that, the mixture was filtered, the filtrate was washed with water (50 mL) and extracted with EtOAc (100 mL), washed with brine and dried over anhydrous sodium sulfate. After being concentrated in vacuo, the residue obtained was purified using column chromatography (Petroleum Ether:EtOAc=1:1) to provide compound 380i (7.07 g, 83.5% yield). LC/MS: Anal. Calcd. For [M+H]⁺ C52H59FN8O7S; 958.42. found 960.

### Step 9

Compound 380j was separated from compound 380i (7.07 g, 7.4 mmol) by the following condition:

Column: Chiral OZ 150×4.6 mm I.D., Sum

Mobile phase: 50% of ethanol (0.05% DEA) in CO₂

Flow rate: 2.0 mL/min

Wavelength: 220 nm

Compound 380j (2.3 g, 75% yield). LC/MS: Anal. Calcd. For [M+H]⁺ C52H59FN8O7S; 958.42. found 960.

### Step 10

Compound 380j (2.3 g, 2 mmol) was added into HCl/dixoane (20 mL). Then the mixture was allowed to stir at 20° C. for 2-3 hours. When the reaction completed, the mixture was concentrated in vacuo to provide the crude product 380k (1.5 g, 83.3% yield). LC/MS: Anal. Calcd. For [M+H]⁺ C47H51FN8O5S; 858.37. found 860.

### Step 11

To a mixture of 380k (1.5 g, 1.75 mmol), cap 1 (0.44 g, 2.3 mmol) and DIPEA (1 mL) in DMF (20 mL) was added HATU (0.67 g, 1.75 mmol). The resulting mixture was allowed to stir at 20° C. The mixture was purified using Pre-HPLC to provide compound 380 (0.9 g, 50% yield). ¹H NMR (MeOD) δ: 8.02 (s, 1H), 7.89-7.94 (m, 1H), 7.78 (d, J=9.8 Hz, 2H), 7.45-7.57 (m, 2H), 7.36 (d, J=11.0 Hz, 1H), 7.29 (br. s., 1H), 7.18 (br. s., 1H), 6.38-6.53 (m, 2H), 5.16-5.27 (m, 2H), 4.70 (d, J=9.4 Hz, 1H), 4.24 (d, J=7.8 Hz, 1H), 3.99-4.16 (m, 2H), 3.88 (dd, J=18.4, 9.8 Hz, 2H), 3.65 (d, J=6.7 Hz, 6H), 3.37-3.48 (m, 2H), 2.55 (d, J=5.5 Hz, 2H), 1.83-2.31 (m, 10H), 1.56 (d, J=12.5 Hz, 1H), 1.37-1.48 (m, 4H), 1.19-1.33 (m, 3H), 1.08 (t, J=5.9 Hz, 5H), 0.83-0.95 (m, 3H), 0.53 (d, J=2.7 Hz, 2H).

The following compound of the present invention was made using the methods described in the Example above and substituting the appropriate reactants or reagents.

| | | | Observed |
|---|---|---|---|
| ID | Structure | Isomer | [M + H]+ |
| 379 | 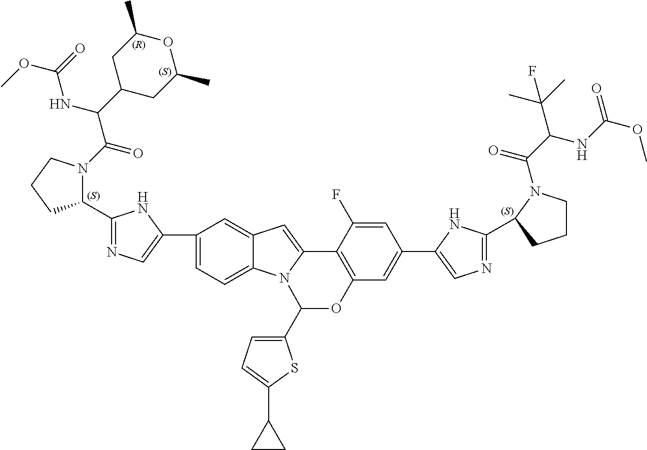 | Isomer 1 | 1034.4 |

### Example 90

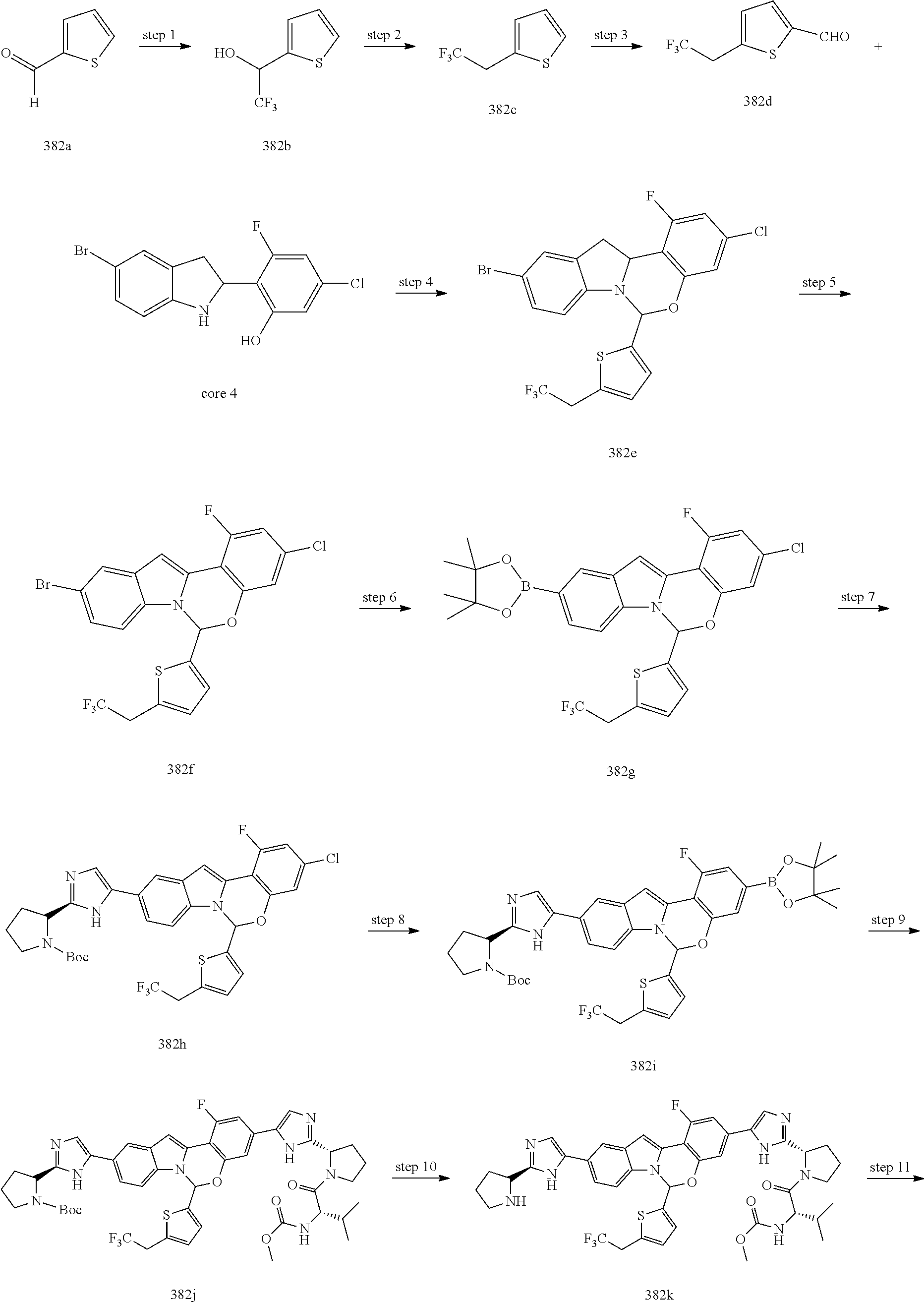

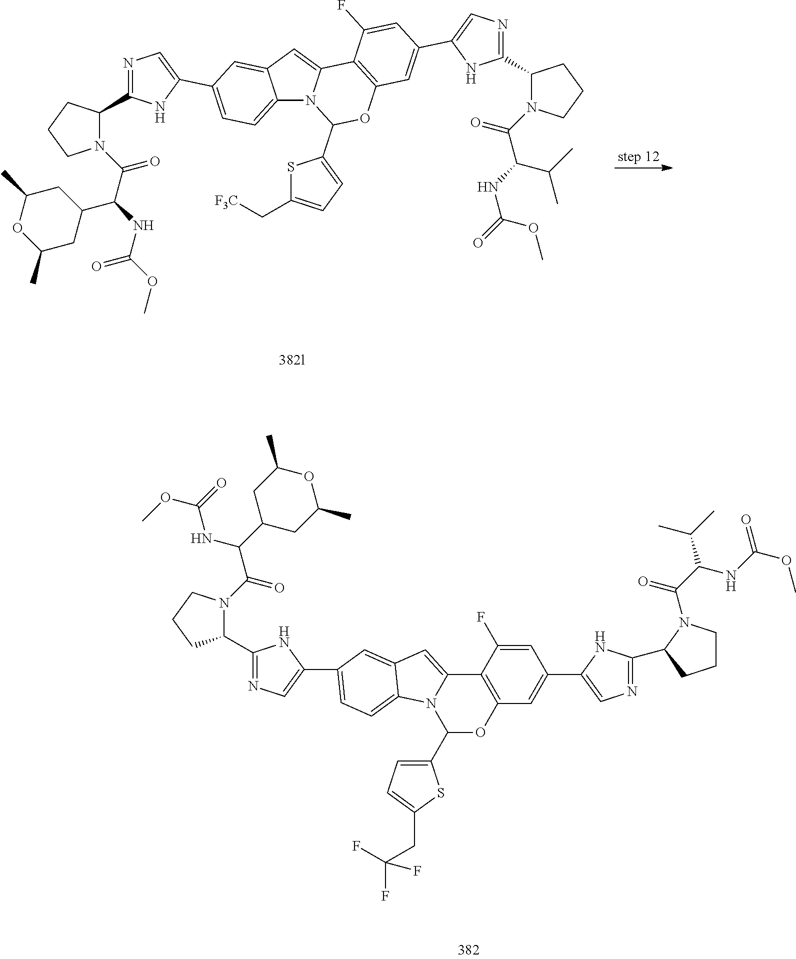

### Step 1

To a suspension of 382a (11.2 g, 0.1 mol) and CsF (1.52 g, 0.01 mol) in DME (70 mL) was added TMSCF₃ (28.4 g, 0.2 mol) dropwise at 0° C. in ice bath. Then the reaction was allowed to stir at 20° C. for 3 hours. The starting material was consumed up. After that, HCl (3N) was added to quench the reaction slowly and it was allowed to stir for another half one hour. The intermediate was used up and the desired product was formed. Extracted with ethyl acetate, dried over anhydrous sodium sulfate, concentrated in vacuo and purified using silica gel chromatography eluting with petroleum/ethyl acetate (100/1-10/1) to provide 382b as a oil (17.2 g, 97% yield). ¹H NMR: 7.39-7.38 (m, 1H), 7.19-7.18 (d, 1H, J=3.6 Hz), 7.05-7.03 (m, 1H), 5.29-5.23 (m, 1H).

### Step 2

To a solution of 382b (6 g, 33.71 mmol) in HOAc (50 mL) and concentrated HCl (25 mL) was added SnCl₂.2H₂O (38 g, 168.5 mmol). Then the mixture was allowed to stir at 80° C. over night. Checked by ¹H NMR and LCMS, the starting material was consumed up. Extracted with CH₂Cl₂, washed with water, saturated sodium hydro carbonate and brine, concentrated in vacuo at ambient temperature to provide the crude product 382c as oil which was used in next step directly (4 g, 69% yield). ¹H NMR: 7.30-7.26 (m, 1H), 7.01-7.00 (m, 2H), 3.68-3.58 (m, 2H).

### Step 3

To a solution of 382c (8 g, 22.89 mmol) in MSA (60 mL) was added urotropine (8.1 g, 57.8 mmol) slowly. After that the reaction was allowed to stir at 75° C. for 3 hours. Detected by TLC, the starting material was consumed up. Extracted with CH₂Cl₂, washed with water, saturated sodium hydrogen carbonate to pH=8 and brine, concentrated in vacuo and purified using silica gel chromatography to provide 382d as oil (450 mg, 5% yield). ¹H NMR: 9.89 (s, 1H), 7.69-7.68 (m, 1H), 7.15-7.14 (m, 1H), 3.69-3.62 (m, 2H).

### Step 4

To a mixture of 382d (1.75 g, 9 mmol) and core 4 (3.2 g, 8.2 mmol) in anhydrous CH₃CN (40 mL) was added TFA (280 mg, 2.45 mmol) at 20° C. The mixture was agitated for 6 hours at 20° C. The reaction mixture became a clear solution and then a solid appeared. The solid was collected by filtration and washed with CH₃CN to provide 382e as a solid (1.8 g, 82% yield). LC/MS: Anal. Calcd. For [M+H]⁺ C₂₁H₁₄BrClF₄NOS; 517.96. found 517.9.

### Step 5

The solution of 382e (1.7 g, 3.0 mmol) in dry toluene (40 mL) was added DDQ (1.1 g, 4.5 mmol). After refluxing for 2 hours, the solvent was removed in vacuo and diluted with EtOAc. The organic layer was washed with saturated Na₂S₂O₃ aqueous and brine, dried over Na₂SO₄, filtered and concentrated in vacuo. The residue obtained was washed with MeOH (20 mL), filtered and the solid just was the product to provide 382f as a solid (1.2 g, 71%). LC/MS: Anal. Calcd. For [M+H]⁺ C₂₁H₁₂BrClF₄NOS; 515.94. found 515.9.

### Step 6

To a solution of 382f (1.2 g, 2.14 mmol) in 1,4-dioxane was added bispinacol borate (650 mg, 2.56 mmol) and Pd(dppf)Cl₂ (78 mg, 0.11 mmol) and KOAc (419 mg, 4.28 mmol). The reaction mixture was allowed to stir under N₂ and heated to 80° C. for 3 hours. After that, the solvent was removed in vacuo, and the residue obtained was purified using column chromatography with silica gel to provide 382g as a solid (1.2 g, 92% yield). LC/MS: Anal. Calcd. For [M+H]⁺ C₂₇H₂₄BClF₄NO₃S; 564.12. found 564.1.

### Step 7

A suspension of 382g (1.2 g, 2.12 mmol), cap 32a (806 mg, 2.55 mmol), Pd(dppf)Cl₂ (78 mg, 0.11 mmol), Na₂CO₃ (449 mg, 4.24 mmol) and in THF/H₂O (10:1, 33 mL) was refluxed at 75° C. for about 15 hours under N₂ atmosphere. After that, the mixture was filtered, the filtrate was washed with water (50 mL) and extracted with EtOAc (100 mL), washed with brine and dried over anhydrous sodium sulfate. After being concentrated in vacuo, the residue obtained was purified using silica gel chromatography to provide 382h as a solid (1.3 g, 91%). LC/MS: Anal. Calcd. For [M+H]⁺ C₃₃H₃₀ClF₄N₄O₃S; 673.17. found 673.2.

### Step 8

To a solution of 382h (1.3 g, 1.93 mmol) in 1,4-dioxane was added bispinacol borate (735 mg, 2.90 mmol) and Pd₂(dba)₃ (174 mg, 0.19 mmol), X-phos (181 mg, 0.38 mmol) and KOAc (378 mg, 3.86 mmol). The reaction mixture was allowed to stir under N₂ and heated to 80° C. for 16 hours. After that, the solvent was removed in vacuo, and the residue obtained was purified using column chromatography with silica gel to provide 382i as a solid (1.4 g, 97% yield). LC/MS: Anal. Calcd. For [M+H]⁺ C₃₉H₄₂BF₄N₄O₅S; 765.29. found 765.2.

### Step 9

A suspension of 382i (1.4 g, 1.83 mmol), cap 31 (819 mg, 2.20 mmol), Pd(dppf)Cl₂ (66 mg, 0.09 mmol), Na₂CO₃ (388 mg, 3.66 mmol) and in THF/H₂O (10:1, 33 mL) was refluxed at 75° C. for about 15 hours under N₂ atmosphere. After that, the mixture was filtered; the filtrate was washed with water (50 mL) and extracted with EtOAc (100 mL), washed with brine and dried over anhydrous sodium sulfate. After being concentrated in vacuo, the residue obtained was purified using silica gel chromatography to provide 382j as a solid (1.2 g, 71% yield). LC/MS: Anal. Calcd. For [M+H]⁺ C₄₇H₅₁F₄N₈O₆S; 931.36. found 931.3.

### Step 10

The compound 382j (1.2 g, 1.29 mmol) was added to a solution of HCl/MeOH (10 mL), then the reaction was allowed to stir at ambient temperature for 4 hours, detected by LC-MS, the reaction was complete. The solvent was removed in vacuo to provide 382k as a solid (1.1 g, 100% yield). LC/MS: Anal. Calcd. For [M+H]⁺ C₄₂H₄₃F₄N₈O₄S; 831.31. found 831.3.

### Step 11

To a solution of 382k (1.1 g, 1.29 mmol), DIEA (666 mg, 5.16 mmol) and cap 4 (316 mg, 1.29 mmol) in DMF (20 mL) was added HATU (490 mg, 1.29 mmol) slowly. Then the mixture was allowed to stir at 25° C. for 3 hours, detected by LCMS, the reaction was complete. It was purified using pre-HPLC to provide 3821 as a solid (800 mg, 70% yield). LC/MS: Anal. Calcd. For [M+H]⁺ C₅₃H₆₀F₄N₉O₈S; 1058.42. found 1058.4.

### Step 12

Compound 382 as a solid was separated from compound 3821 (800 mg) by the following method:

Column: Chiralcel OD-3 150×4.6 mm I.D., 3 μm

Mobile phase: 40% of ethanol(0.05% DEA) in CO₂

Flow rate: 2.4 mL/min

Wavelength: 340 nm

Compound 382 (250 mg, 62.5% yield). ¹H-NMR: δ 8.02 (s, 1H), 7.88 (d, J=7.43 Hz, 2H), 7.72-7.79 (m, 1H), 7.47-7.60 (m, 2H), 7.26-7.41 (m, 2H), 7.16 (d, J=2.35 Hz, 1H), 6.80 (d, J=3.13 Hz, 1H), 6.54-6.66 (m, 1H), 5.20 (dt, J=15.16, 7.48 Hz, 2H), 4.16-4.29 (m, 2H), 4.07 (br. s., 2H), 3.86 (dd, J=15.46, 7.63 Hz, 2H), 3.50-3.71 (m, 8H), 3.37-3.45 (m, 2H), 2.44-2.62 (m, 2H), 1.95-2.31 (m, 8H), 1.54 (d, J=11.74 Hz, 1H), 1.30 (d, J=12.13 Hz, 1H), 1.04-1.17 (m, 6H), 0.84-0.99 (m, 8H). LC/MS: Anal. Calcd. For [M+H]⁺ C₅₃H₆₀F₄N₉O₈S:1058.42. found 1058.4.

The following compounds of the present invention were made using the methods described in the Example above and substituting the appropriate reactants or reagents.

| | | | Observed |
|---|---|---|---|
| ID | Structure | Isomer | [M + H]+ |
| 381 | 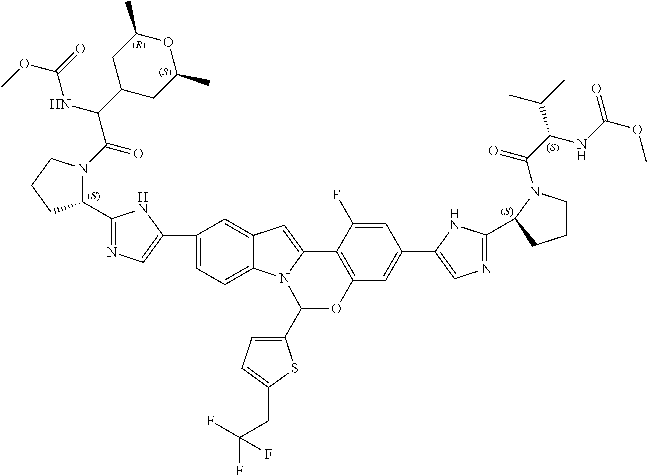 | Isomer 1 | 1058.4 |

### Example 91

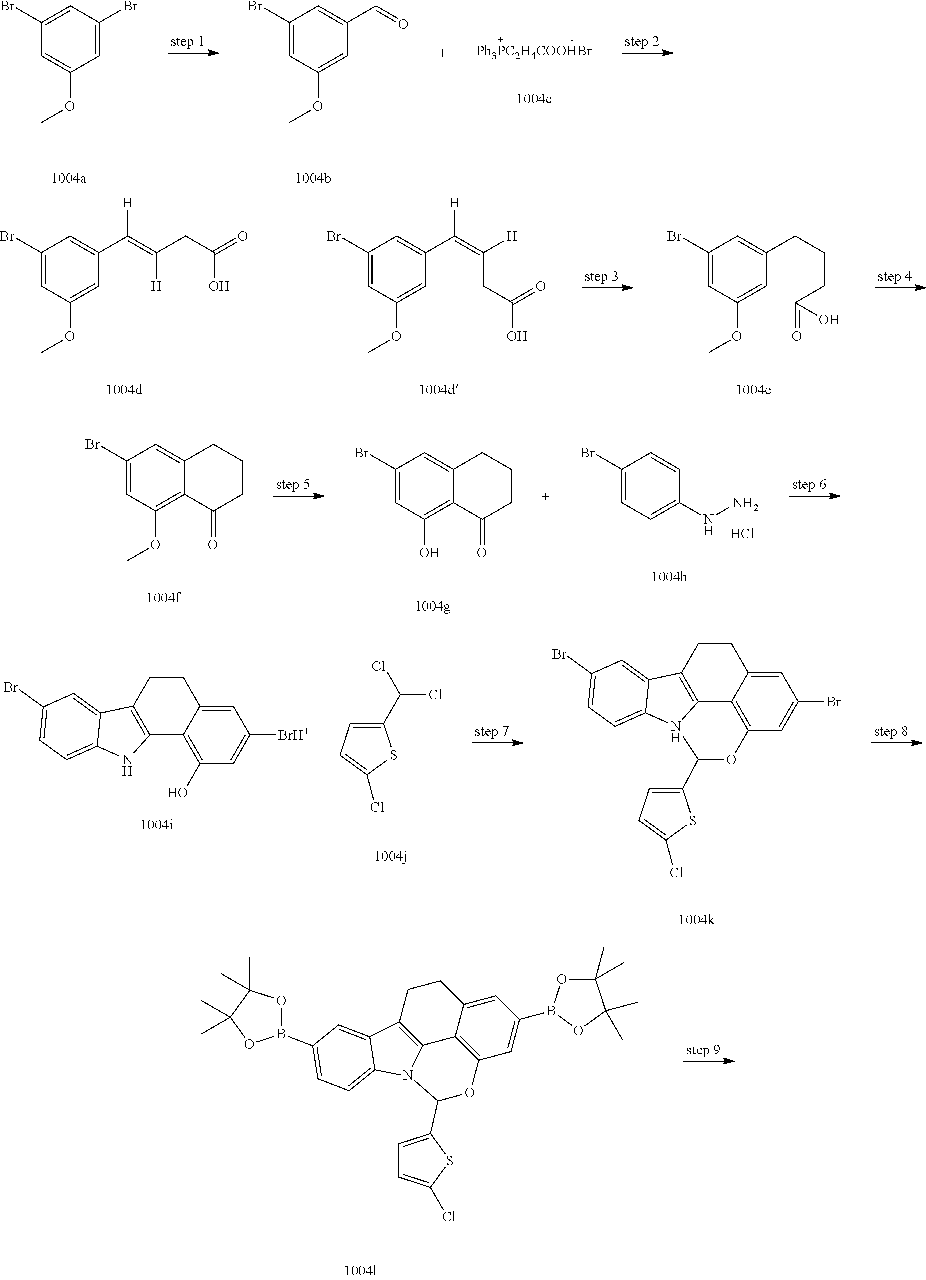

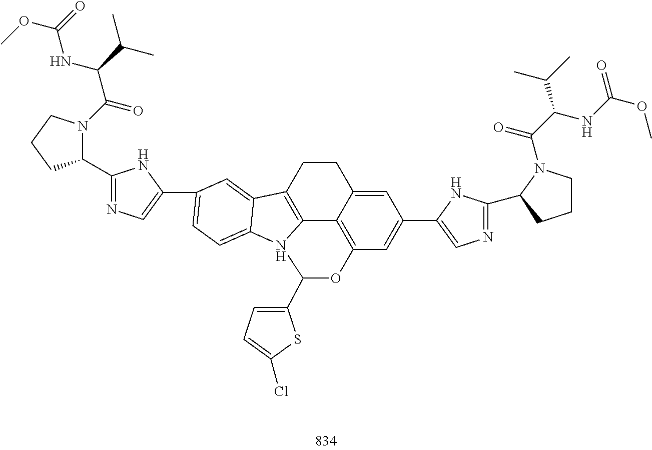

### Step 1

To a solution of compound 1004a (26.6 g, 0.1 mmol) in anhydrous THF (300 mL) was added n-BuLi (44 mL, 0.11 mmol) dropwise under N₂ at −78° C. When the addition was finished, the mixture was allowed to stir for an additional 1 hour at −78° C. DMF (10 g, 0.15 mol) was added below −70° C. The mixture was allowed to stir at −78° C. for 2 hours. Citric acid was added to the reaction mixture. The mixture was poured into water. The aqueous was extracted with ethyl acetate.

The organic layers were dried over anhydrous sodium sulfate and concentrated in vacuum. The reaction mixture was purified using SiO₂ chromatography, eluting with petroleum ether:ethyl acetate (10/15/1) to provide the title compound 1004b (10.0 g, 46.5% yield). ¹H NMR (400 MHz, CDCl₃-d) δ: 9.89 (s, 1H), 7.58-7.55 (m, 1H), 7.32-7.28 (m, 2H), 3.85 (s, 3H).

### Step 2

To a solution of compound 1004b (4.3 g, 20 mmol), compound 1004c (8.7 g, 21 mmol) in anhydrous THF (200 mL) was added t-BuOK (42 mL, 42 mmol, 1.0 M in THF) over a period of 1 h at 0° C. After the addition was finished, the mixture was allowed to stir for an additional 0.5 hour at 0° C. The mixture was allowed to stir at 20° C. for 10 hours. The mixture was poured into ice-water. The aqueous layer was adjusted to pH 2 with conc. HCl and extracted with ethyl acetate. The combined organic layer was dried over anhydrous sodium sulfate and concentrated in vacuum to provide the crude compound 1004d and 1004d′ (5.0 g, 92.6% yield). LC/MS: Anal. Calcd. For [M+H]⁺ C11H11BrO3; 269.99. found 271.11.

### Step 3

To a solution of the mixture of the compound 1004d and 1004d′ (5.0 g, 18 mmol) in methanol (100 mL) was added 10% Rh(PPh₃)₃C1 (0.5 g). The mixture was allowed to stir under H₂ (balloon) at 50° C. for 1 h and filtered through celite. The filtrate was concentrated in vacuum to provide the compound 1004e (4.5 g, 88.2% yield). ¹H NMR (400 MHz, DMSO-d6) δ: 6.93 (s, 2H), 6.79-6.69 (m, 1H), 3.71 (s, 3H), 2.51 (m, 2H), 2.13 (s, 2H), 1.78-1.64 (m, 2H).

### Step 4

A solution of compound 1004e (9.0 g, 33 mmol) in 100 mL PPA was heated at 100° C. for 2 hours. The reaction mixture was poured into ice-water and extracted with ethyl acetate. The combined organic layer was washed with saturated NaHCO₃ solution and brine. The organic solution was dried over Na₂SO₄, concentrated in vacuum to provide compound 1004f (4.8 g, 57.3% yield). ¹H NMR (400 MHz CDCl3) δ: 7.06-6.97 (m, 1H), 6.62 (s, 1H), 3.77 (s, 3H), 2.86 (br, 2H), 2.58 (s, 2H), 1.99 (br, 2H).

### Step 5

A solution of compound 1004f (4.8 g, 18 mmol) in 100 mL of anhydrous CH₂Cl₂ was treated with BBr₃ (22.5 g, 90 mmol) at −15° C. to −5° C. for 4 hours. The mixture was poured into 600 mL of ice-water, and the organic layer was washed with brine and dried over anhydrous sodium sulfate. After concentrated in vacuum, the residue was purified using SiO₂ chromatography, eluting with petroleum ether:ethyl acetate (20/1˜5/1) to provide the product 1004g (1.7 g, 39.5% yield). 1H NMR (400 MHz CDCl3) δ: 12.43 (s, 1H), 6.93 (s, 1H), 6.83-6.79 (m, 1H), 2.86-2.76 (m, 2H), 2.61 (s, 2H), 2.07-1.97 (m, 2H).

### Step 6

A suspension of compound 1004g (720 mg, 30 mmol), compound 1004h (1.0 g, 4.5 mmol), in EtOH-H₂O (40 mL/4 mL) was refluxed for one day. After cooled to room temperature, the mixture was washed with saturated NaHCO₃, brine and dried over anhydrous sodium sulfate. After concentrated in vacuum, the residue was purified using Pre-HPLC to provide the product 1004i (400 mg, 33.3% yield). ¹H NMR (400 MHz CDCl3) δ: 9.30 (br. s., 1H), 7.68-7.60 (m, 1H), 7.26-7.19 (m, 2H), 7.02 (s, 1H), 6.84 (s, 1H), 5.94-5.69 (br. s., 1H), 3.00 (t, J=8.0 Hz, 2H), 2.90 (t, J=8.0 Hz, 2H).

### Step 7

A mixture of compound 1004i (117 mg, 0.3 mmol), compound 1004j (121 mg, 0.6 mmol), and Cs₂CO₃ (324 mg, 0.9 mmol) in 15 mL DMF was heated to 100° C. overnight. The mixture was concentrated in vacuum and dissolved with DCM and water. The aqueous phase was extracted with DCM. The combined organic layers were washed with brine, dried over Na₂SO₄ and concentrated in vacuum. The resulting residue was purified using Prep-HPLC to provide compound 1004k (20 mg, 12.8% yield). LC/MS: Anal. Calcd. For [M+H]⁺ C21H12Br2ClNOS; 518.87. found 522.65.

### Step 8

To a solution of compound 1004k (20 mg, 0.04 mmol) in 1,4-dioxane (10 mL) was added bis pinacol borate (24 mg, 0.09 mmol) and Pd(dppf)Cl₂ (3 mg, 0.004 mmol) and KOAc (19 mg, 0.2 mmol). The reaction mixture was allowed to stir under N₂ and heated to 110° C. for 2 hours. After that, the solvent was removed under vacuum, and the residue was purified using SiO₂ chromatography, eluting with petroleum ether:ethyl acetate (20/1˜10/1) to provide the compound 10041 (18 mg, 75% yield). LC/MS: Anal. Calcd. For [M+H]⁺ C33H36B2ClNO5S; 615.22 found 616.2.

### Step 9

A suspension of compound 10041 (18 mg, 0.03 mmol), methyl ((S)-1-((S)-2-(5-bromo-1H-imidazol-2-yl)pyrrolidin-1-yl)-3-methyl-1-oxobutan-2-yl)carbamate (26 mg, 0.107 mmol), Pd(dppf)Cl₂ (2 mg, 0.003 mmol), Na₂CO₃ (16 mg, 0.15 mmol) and in THF-H₂O (10-2 mL) was refluxed at 90° C. overnight under N₂ protection. After that, the mixture was filtered through elite, and the filtrate was diluted with EtOAc (50 mL), washed with brine and dried over anhydrous sodium sulfate. After concentrated in vacuum, the residue was purified using Pre-HPLC to provide compound 834 (1 mg, 4% yield). ¹H NMR (MeOD 400 MHz): δ: 7.93-7.84 (m, 1H), 7.71 (s, 1H), 7.64-7.57 (m, 1H), 7.45 (s, 2H), 7.34 (s, 1H), 7.29-7.21 (m, 1H), 7.07 (s, 2H), 6.85-6.77 (m, 1H), 5.26-5.14 (m, 2H), 4.50-4.41 (m, 2H), 4.26-4.13 (m, 2H), 4.12-4.00 (m, 2H), 3.88-3.56 (m, 6H), 2.88-2.75 (m, 2H), 2.56-2.43 (m, 2H), 2.30-1.89 (m, 8H), 1.31-1.26 (m, 2H), 1.20 (m, 8H), 0.87 (m, 4H). LC/MS: Anal. Calcd. For [M+H]⁺ C49H54ClN9O7S:948.55. found (M/2+H): 948.6.

### Example 92

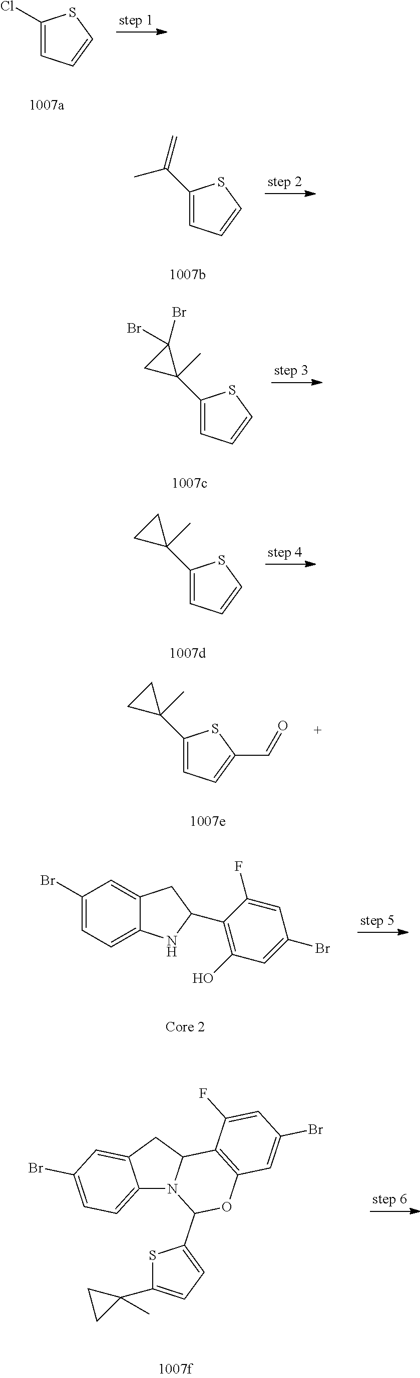

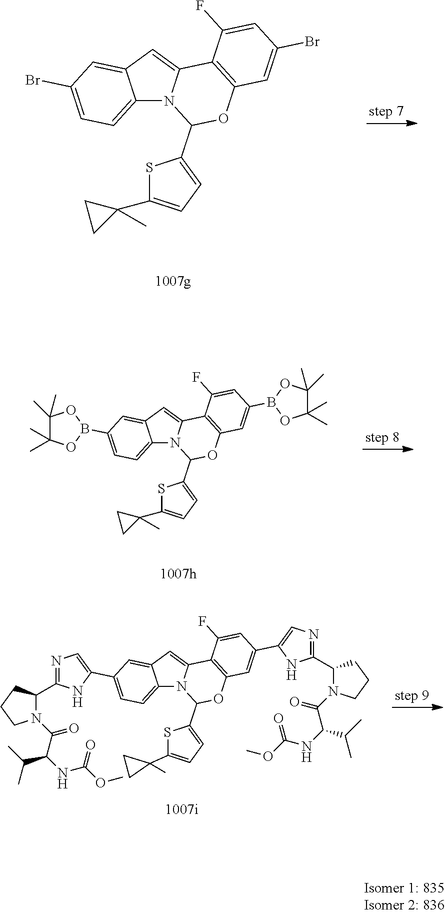

### Step 1

The mixture of 1007a (3.86 g, 32.5 mmol), potassium trifluoro(prop-1-en-2-yl)borate (14.4 g, 97.6 mmol), Pd(OAc)₂ (438 mg, 1.95 mmol), n-BuPACl₂ (1.4 g, 3.9 mmol) and Cs₂CO₃ (31.8 g, 97.6 mmol) in PhMe/H₂O (10:1, 110 mL) was allowed to stir at 100° C. under N₂ overnight. After filtration, the mixture was extracted with EtOAc. The organic layer was washed with brine, dried over Na₂SO₄, concentrated to provide 1007b (4 g, 99.3%).

### Step 2

To a solution of 1007b (4 g, 32.3 mmol) in DCM (100 mL) was added NaOH (6.46 g, 80.75 mmol), N,N,N-trimethylhexadecan-1-aminium chloride (1.04 g, 3.23 mmol) and Bromoform (16.32 g, 64.52 mmol). The mixture was allowed to stir at 70° C. overnight. After cooling to room temperature, the reaction mixture was adjusted to pH=7 by the addition of HCl (1 M), extracted with DCM. The organic layer washed with brine, dried over Na₂SO₄. After filtration and concentration, the residue was purified using SiO₂ chromatography, eluting with petroleum ether:ethyl acetate (100/1˜10/1) to provide 1007c (4.17 g, 43.6%).

### Step 3

LiBH₄ (1.23 g, 45.1 mmol) was added in portions to a stirred solution of Cp₂TiCl₂ (1.12 g, 4.51 mmol) and 1007c (2.67 g, 9.02 mmol) in THF (100 mL) at room temperature under N₂. The mixture was allowed to stir at 70° C. overnight. After cooling to room temperature, the reaction mixture was added slowly to 1M HCl, extracted with EtOAc, dried over Na₂SO₄, concentrated to provide crude 1007d.

### Step 4

To a solution of crude 1007d (1.5 g, 10.87 mmol) in THF (30 mL) was added a 2.5 M solution of t-BuLi (5.2 mL, 13.04 mmol) at −60° C. The mixture was agitated for 1 h at this temperature then DMF (1.59 g, 21.74 mmol) was added. The mixture was allowed to stir at this temperature for 1 h, before quenched with NH₄Cl solution and extracted with EtOAc. The organic layer was dried over Na₂SO₄, concentrated in vacuum and purified using SiO₂ chromatography, eluting with petroleum ether:ethyl acetate (100/1˜10/1) to provide 1007e (340 mg, 15.7%).

### Step 5

To a mixture of 1007e (440 mg, 2.63 mmol) and Core2 (784 mg, 2.03 mmol) in anhydrous CH₃CN (10 mL) was added TFA (69.4 mg, 0.61 mmol) at room temperature. The mixture was agitated for 3h at room temperature. The reaction mixture became a clear solution and then solid appeared. The solid was collected by filtration and washed with CH₃CN to provide compound 1007f (485 mg, 44.9%).

### Step 6

To a solution of 1007f (485 mg, 0.91 mmol) in dry toluene (20 mL) was added DDQ (309 mg, 1.36 mmol). After refluxing for 2 hours, the solvent was removed and diluted with EtOAc. The organic layer was washed with saturated Na₂S₂O₃ aqueous and brine, dried over Na₂SO₄, filtered and concentrated. The residue was washed with MeOH (50 mL), filtered to provide compound 1007g (483 mg, 93%).

### Step 7

A suspension of 1007g (450 mg, 0.844 mmol), bis(pinacolato)diboron (536 mg, 2.11 mmol), KOAc (331 mg, 3.38 mmol) and Pd(dppf)Cl₂ (62 mg, 0.08 mmol) in dioxane (10 mL) was allowed to stir at 100° C. for 2 hours under N₂ protection. The reaction mixture was cooled and concentrated in vacuum, the residue was purification by SiO₂ chromatography, eluting with petroleum ether:ethyl acetate (30:1) to provide 1007h (480 mg, 90.7%).

### Step 8

A suspension of 1007h (480 mg, 0.766 mmol), methyl ((S)-1-((S)-2-(5-bromo-1H-imidazol-2-yl)pyrrolidin-1-yl)-3-methyl-1-oxobutan-2-yl)carbamate (628 mg, 1.68 mmol), Pd(dppf)₂Cl₂ (59 mg, 0.08 mmol) and Na₂CO₃ (325 mg, 3.1 mmol) in THF/H₂O (8:1, 30 mL) was allowed to stir at 80° C. for overnight under N₂ protection. After that, the mixture was washed with water and extracted with ethyl acetate, washed with brine and dried over anhydrous sodium sulfate. After concentrated in vacuum, the residue was purified with pre-HPLC to provide 1007i (200 mg, 27.2%).

### Step 9

The two diastereoisomers of 1007i (200 mg) were separated by SFC by using the following conditions to provide 835 (70 mg, 35%) and 836 (60 mg, 30%).

Column: Chiralcel OZ-3 150×4.6 mm I.D.

Mobile phase: 50% of ethanol (0.05% DEA) in CO₂

Flow rate: 2.0 mL/min

Wavelength: 220 nm

835: ¹H NMR (400 MHz, METHANOL-d4) δ: 8.07 (s, 1H), 8.02-7.97 (m, 1H), 7.85 (s, 1H), 7.79 (s, 1H), 7.60-7.50 (m, 2H), 7.43 (d, J=11.0 Hz, 1H), 7.36 (s, 1H), 7.25 (br. s., 1H), 6.53 (dd, J=3.5, 18.4 Hz, 2H), 5.26 (td, J=7.2, 14.2 Hz, 2H), 4.25 (t, J=8.0 Hz, 2H), 4.13 (br. s., 2H), 3.94-3.85 (m, 2H), 3.75-3.57 (m, 6H), 2.59 (br. s., 2H), 2.30 (br. s., 2H), 2.20 (br. s., 4H), 2.12-2.03 (m, 2H), 1.35 (s, 3H), 1.02-0.89 (m, 12H), 0.79 (s, 4H). LC/MS: Anal. Calcd. For [M+H]⁺ C51H58FN9O7S; 960.13. found 960.4.

836: ¹H NMR (400 MHz, METHANOL-d4) δ: 8.00 (s, 1H), 7.91 (s, 1H), 7.80 (s, 1H), 7.75 (s, 1H), 7.50 (q, J=8.7 Hz, 2H), 7.36 (d, J=10.6 Hz, 1H), 7.30 (s, 1H), 7.18 (br. s., 1H), 6.50 (d, J=3.5 Hz, 1H), 6.43 (d, J=3.5 Hz, 1H), 5.25-5.15 (m, 2H), 4.20 (t, J=7.2 Hz, 2H), 4.08 (br. s., 2H), 3.87-3.80 (m, 2H), 3.63 (s, 6H), 2.53 (d, J=8.2 Hz, 2H), 2.25 (d, J=5.1 Hz, 2H), 2.18-2.09 (m, 4H), 2.05-1.99 (m, 2H), 1.30 (s, 3H), 0.98-0.84 (m, 12H), 0.75 (s, 4H). LC/MS: Anal. Calcd. For [M+H]⁺ C51H58FN9O7S; 960.13. found 960.8.

### Example 93

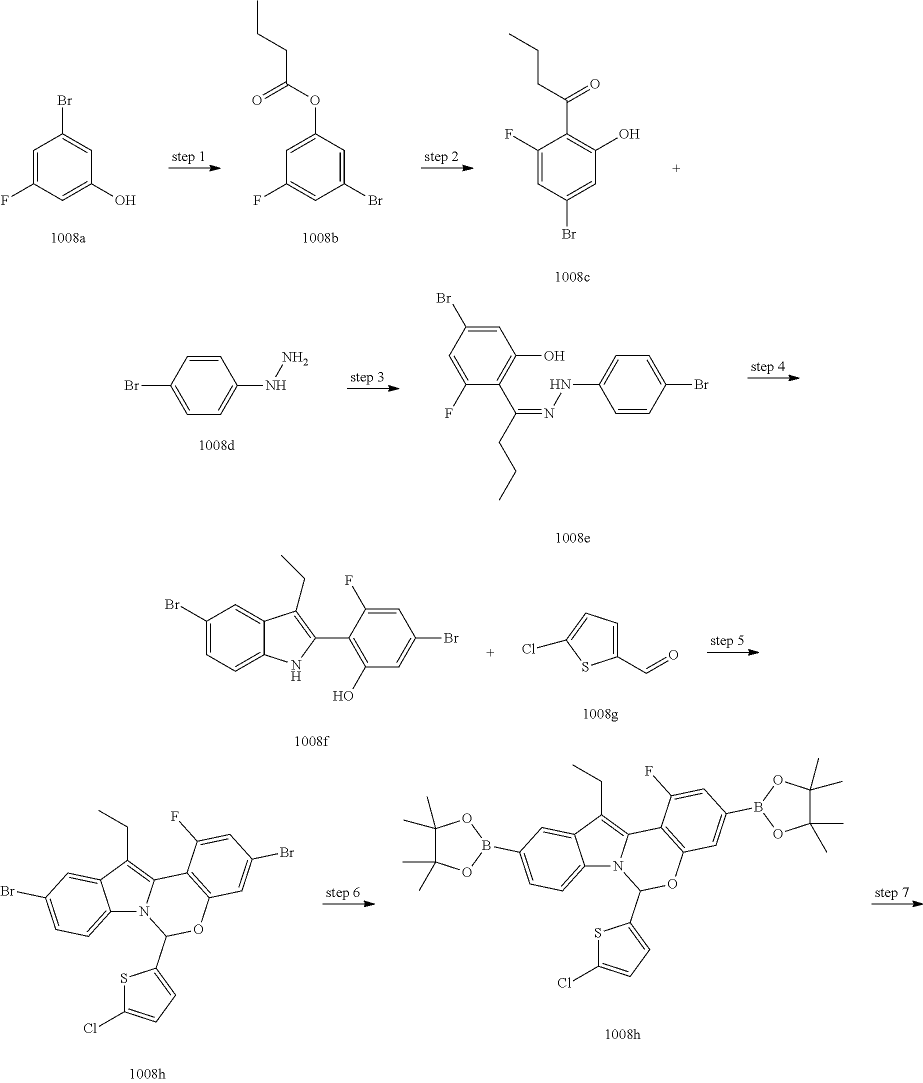

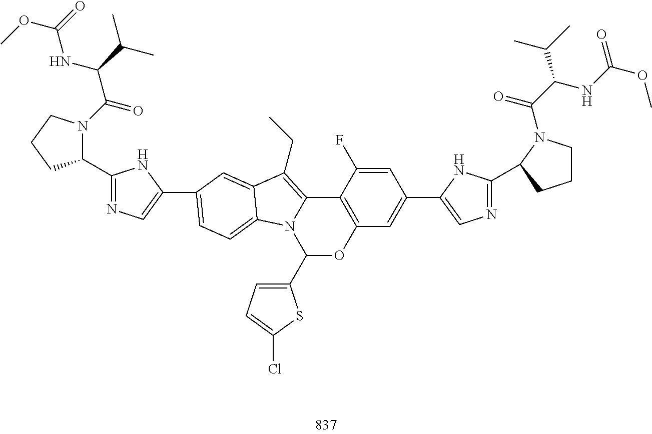

### Step 1

To a solution of compound 1008a (15.8 g, 0.083 mol), Et₃N (12.1 g, 0.12 mol) in DCM (125 mL) was added butyryl chloride (10.6 g, 0.1 mol) at 0° C. The mixture was allowed to stir at the same temperature for 1 h. The crude product was washed with 1N HCl, NaHCO₃, and brine. The organics were dried over Na₂SO₄, filtered, and the filtrate was concentrated, evaporated in vacuum to provide compound 1008b (18.5 g, 85.6% yield).

### Step 2

The compound 1008b was heated at 100° C., then AlCl₃ (28 g, 0.21 mol) was added, the temperature was heated to 140° C. for 1 h. The mixture was poured into ice water and extracted with DCM. The organic layer was washed with NaHCO₃ and NaCl, dried over Na₂SO₄, evaporated in vacuum to provide compound 1008c (8.56 g, 46.3% yield).

### Step 3

To a solution of compound 1008c (8.56 g, 0.033 mol), 1008d (8.85 g, 0.04 mol) in MeOH (90 mL) was added AcOH (9 mL), the mixture was allowed to stir at 60-64° C. for 15 hours. The solvent was removed to provide crude compound 1008e (14 g, 100% yield).

LC/MS: Anal. Calcd. For [M+H]⁺ C16H15Br2FN2O; 427.95. found 431.1.

### Step 4

Compound 1008e (16 g, 0.037 mol) in CH₃SO₃H (80 mL) was stirring at 85° C. for 2 hours. The mixture was poured into ice water, extracted with MTBE. The organic layer was separated and washed with NaHCO₃ and NaCl solution, dried over anhydrous Na₂SO₄, and concentrated under reduced pressure. The residue was purified using SiO₂ chromatography, eluting with petroleum ether:ethyl acetate (3:1) to provide 1008f (9.5 g, 59.7% yield). LC/MS: Anal. Calcd. For [M+H]⁺ C16H12Br2FNO 412.93. found 413.6.

### Step 5

Compound 1008f (0.5 g, 1.21 mmol), compound 1008g (0.194 g, 1.33 mmol) and TosCl (69 mg, 0.363 mmol) in toluene (10 mL) was allowed to stir at 130° C. for 10 hours. After that, the solvent was removed under vacuum, and the residue was purified using SiO₂ chromatography, eluting with petroleum ether:ethyl acetate (3:1) to provide the product 1008h (180 mg, 27.5% yield). LC/MS: Anal. Calcd. For [M+H]⁺ C21H13Br2ClFNOS; 542.87. found 543.6.

### Step 6

To a solution of compound 1008h (230 mg, 0.43 mmol) in 1,4-dioxane was added bis pinacol borate (229 mg, 0.90 mmol) and Pd(dppf)Cl₂ (31 mg, 0.043 mmol) and KOAc (253 mg, 2.58 mmol). The reaction mixture was allowed to stir under N₂ and heated to 110° C. for overnight. After that, the solvent was removed under vacuum, and the residue was purified using SiO₂ chromatography, eluting with petroleum ether:ethyl acetate (2:1) to provide the product 1008i (180 mg, 65.9% yield). LC/MS: Anal. Calcd. For [M+H]⁺ C33H37B2ClFNO5S; 635.23. found 635.6.

### Step 7

A suspension of compound 1008i (180 mg, 0.28 mmol), methyl ((S)-1-((S)-2-(5-bromo-1H-imidazol-2-yl)pyrrolidin-1-yl)-3-methyl-1-oxobutan-2-yl)carbamate (220 mg, 0.59 mmol), Pd(dppf)Cl₂ (20 mg, 0.028 mmol), Na₂CO₃ (178 mg, 1.68 mmol) and in THF/H₂O (5:1, 20 mL) was refluxed at 90° C. overnight under N₂ protection. After that, the mixture was filtered, and the filtrate was washed with water and extracted with EtOAc. The organic layer was washed with brine and dried over anhydrous sodium sulfate. After concentrated in vacuum, the residue was purification by SiO₂ chromatography, eluting with ethyl acetate to provide compound 837 (240 mg, 88.9% yield). ¹H NMR (400 MHz, METHANOL-d4) δ: 8.04-8.11 (m, 1H), 7.79 (m, 3H), 7.52-7.62 (m, 2H), 7.31-7.43 (m, 2H), 6.74 (m, 1H), 6.50-6.58 (m, 1H), 5.13-5.28 (m, 2H), 4.21 (m, 2H), 4.00-4.14 (m, 2H), 3.78-3.90 (m, 2H), 3.64 (s, 6H), 3.07-3.17 (m, 2H), 2.42-2.67 (m, 3H), 2.15 (m, 7H), 1.35 (m, 3H), 0.91 (m, 12H). LC/MS: Anal. Calcd. For [M+H]⁺ C49H55ClFN9O7S; 968.55. found: 968.8.

### Example 94

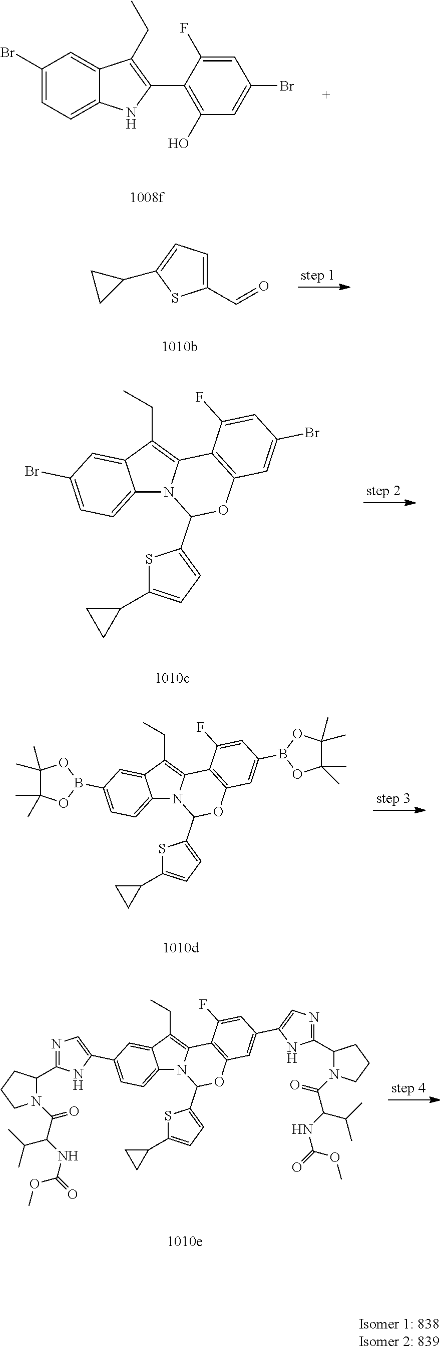

### Step 1

Compound 1008f was made in Example 93. Compound 1008f (1 g, 2.42 mmol), 1010b (0.402 g, 2.66 mmol) and TosCl (139 mg, 0.73 mmol) in xylene (20 mL) was allowed to stir at 170° C. for 16 hours. After that, the solvent was removed under vacuum, and the residue was purified using SiO₂ chromatography, eluting with petroleum ether:ethyl acetate (3:1) to provide compound 1010c (680 mg, 51.5% yield). LC/MS: Anal. Calcd. For [M+H]⁺ C24H18Br2FNOS:546.94. found 546.6.

### Step 2

To a solution of compound 1010c (680 mg, 1.24 mmol) in 1,4-dioxane was added bis pinacol borate (661 mg, 2.60 mmol) and Pd(dppf)Cl₂ (88 mg, 0.12 mmol) and KOAc (729 mg, 7.44 mmol). The reaction mixture was allowed to stir under N₂ and heated to 110° C. for overnight. After that, the solvent was removed under vacuum, and the residue was purified using SiO₂ chromatography, eluting with petroleum ether:ethyl acetate (3:1) to provide compound 1010d (770 mg, 96.8% yield). LC/MS: Anal. Calcd. For [M+H]⁺ C36H42B2FNO5S:641.30. found 642.1.

### Step 3

A suspension of compound 1010d (770 mg, 1.20 mmol), methyl ((S)-1-((S)-2-(5-bromo-1H-imidazol-2-yl)pyrrolidin-1-yl)-3-methyl-1-oxobutan-2-20 yl)carbamate (940 mg, 2.52 mmol), Pd(dppf)Cl₂ (88 mg, 0.12 mmol), Na₂CO₃ (763 mg, 7.20 mmol) and in THF/H₂O (5:1, 30 mL) was refluxed at 90° C. overnight under N₂ protection. After that, the mixture was filtered, and the filtrate was washed with water and extracted with EtOAc. The organic layer was washed with brine and dried over anhydrous sodium sulfate. After concentrated in vacuum, the residue was purification by SiO₂ chromatography, eluting with ethyl acetate to provide compound 1010e (920 mg, 78.6% yield). LC/MS: Anal. Calcd. For [M+H]⁺ C52H60FN9O7S: 974.15. found 974.6.

### Step 4

Compound 838 (the fast peak) and compound 839 (the second peak) were got from compound 1010e (430 mg) by SFC by using the following conditions:

Instrument: Thar SFC

Column: OD-3 150×4.6 mm I.D.

Mobile phase: 40% of methanol (0.05% DEA) in CO₂

Flow rate: 2.5 mL/min

Back pressure: 100 bar

Column temperature: 35° C.

Wavelength: 340 nm

Compound 838 (160 mg, 37.2% yield). ¹H NMR (400 MHz, METHANOL-d4) δ: 8.06-8.12 (m, 1H), 7.91-7.97 (m, 1H), 7.78-7.83 (m, 1H), 7.71-7.77 (m, 1H), 7.54-7.60 (m, 1H), 7.46-7.52 (m, 1H), 7.36-7.42 (m, 1H), 7.32-7.36 (m, 1H), 6.47-6.56 (m, 2H), 5.17-5.30 (m, 2H), 4.18-4.27 (m, 2H), 4.05-4.16 (m, 2H), 3.81-3.91 (m, 2H), 3.66 (s, 6H), 3.07-3.19 (m, 2H), 2.50-2.62 (m, 2H), 2.23-2.32 (m, 2H), 2.11-2.22 (m, 4H), 2.01-2.10 (m, 2H), 1.90-1.99 (m, 1H), 1.36 (m, 3H), 0.74-1.11 (m, 14H), 0.52-0.61 (m, 2H). LC/MS: Anal. Calcd. For [M+H]⁺ C52H60FN9O7S; 974.15. found 974.6.

Compound 839 (210 mg, 48.8% yield). ¹H NMR (400 MHz, METHANOL-d4) δ: 8.07-8.11 (m, 1H), 7.92-7.98 (m, 1H), 7.78-7.81 (m, 1H), 7.72-7.76 (m, 1H), 7.55-7.60 (m, 1H), 7.47-7.52 (m, 1H), 7.36-7.42 (m, 1H), 7.31-7.35 (m, 1H), 6.51 (m, 2H), 5.18-5.29 (m, 2H), 4.19-4.28 (m, 2H), 4.05-4.16 (m, 2H), 3.83-3.91 (m, 2H), 3.65 (s, 6H), 3.08-3.18 (m, 2H), 2.51-2.61 (m, 2H), 2.23-2.31 (m, 2H), 2.13-2.22 (m, 3H), 2.02-2.10 (m, 2H), 1.90-1.98 (m, 1H), 1.31-1.39 (m, 3H), 0.85-1.01 (m, 14H), 0.51-0.59 (m, 2H). LC/MS: Anal. Calcd. For [M+H]⁺ C52H60FN9O7S; 974.15. found 974.6.

The following compounds were prepared according to the procedure described above.

| | | | Observed |
|---|---|---|---|
| ID | Structures | Isomer | [M + H]+ |
| 840 841 842 | 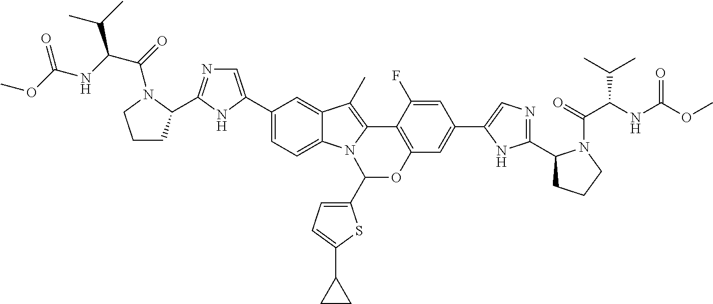 | Racemic Isomer 1 Isomer 2 | 961.7 961.2 961.3 |

### Example 95

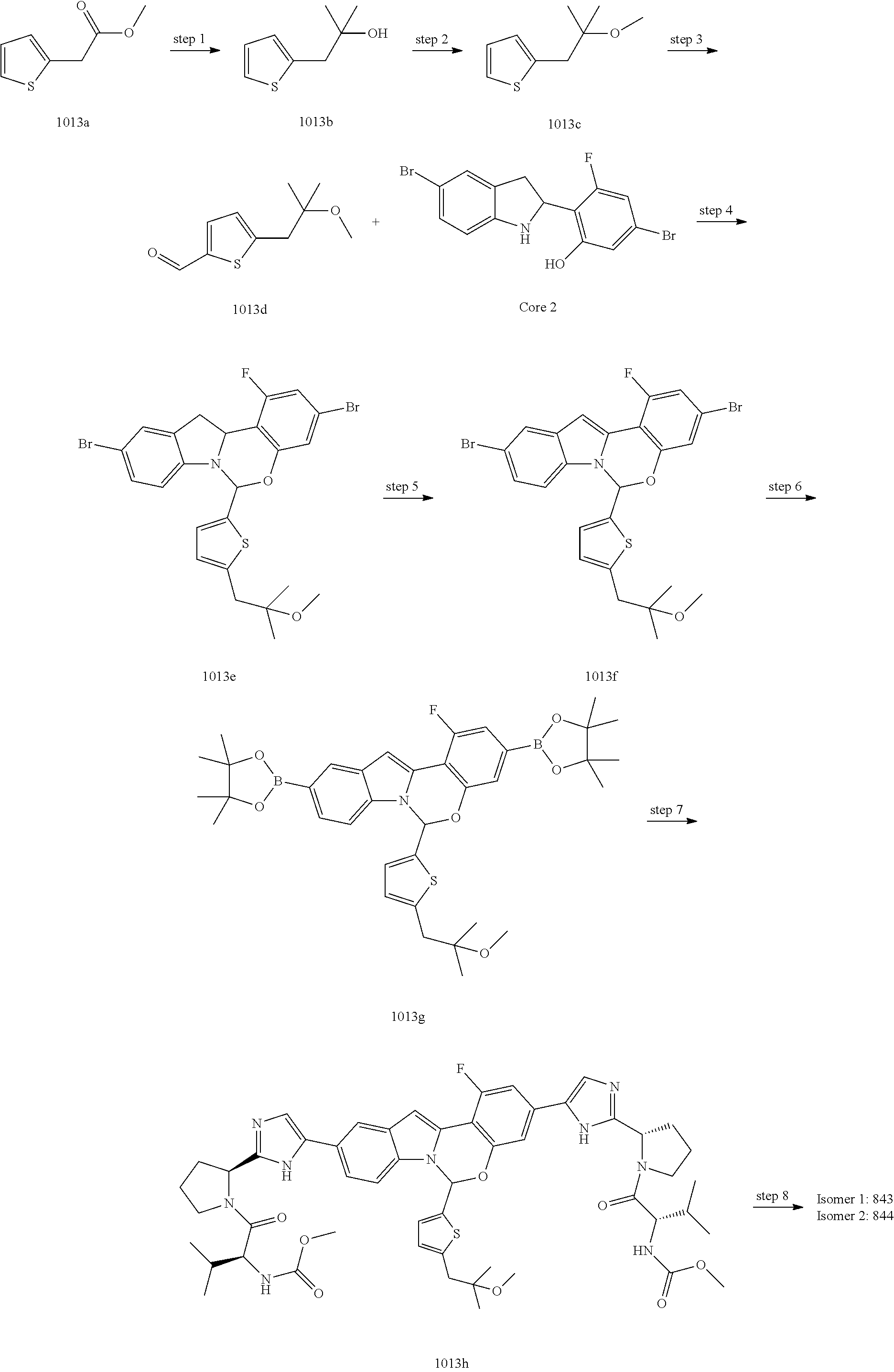

### Step 1

MeMgBr (51.5 mL, 154.6 mmol) was added dropwise to a stirred solution of 1013a (8.04 g, 51.54 mmol) in THF (100 mL) at 0° C. under N₂ protection. The mixture was allowed to warm to room temperature overnight. After that, the reaction mixture was added to a solution of HCl (1 M, 50 mL), extracted with EtOAc, dried over Na₂SO₄, purified using SiO₂ chromatography, eluting with petroleum ether: ethyl acetate (100/1˜30/1) to provide 1013b (3.85 g, 47.9%).

### Step 2

To a solution of 1013b (13.9 g, 89.4 mmol) in THF (200 mL) was added NaH (7.16 g, 178.9 mmol) in portions at 0° C. The mixture was allowed to stir at 0° C. for 30 min, and then to the mixture was added MeI (25.41 g, 178.9 mmol). The mixture was allowed to stir at room temperature overnight before poured into water (200 mL), extracted with EA, dried over Na₂SO₄, evaporated to provide 1013c (14.9 g, 98%).

### Step 3

To a solution of crude 1013c (4.8 g, 28.2 mmol) in THF (100 mL) was added a 2.5 M solution of t-BuLi (13.6 ml, 33.9 mmol) at −60° C. The mixture was agitated for 1 h at this temperature then DMF (4.17 g, 56.4 mmol) was added. The mixture was allowed to stir at this temperature for 1 h, before quenched with NH₄Cl saturate solution and extracted with EtOAc. The organic layer was dried over Na₂SO₄, and concentrated in vacuum and purified using SiO₂ chromatography, eluting with petroleum ether:ethyl acetate (100/1-10/1) to provide 1013d (5.3 g, 95%).

### Step 4

To a mixture of 1013d (1 g, 5.05 mmol) and Core2 (1.5 g, 3.88 mmol) in anhydrous CH₃CN (15 mL) was added TFA (133 mg, 1.16 mmol) at room temperature. The mixture was agitated for 3h at room temperature. The reaction mixture became a clear solution and then solid appeared. The solid was collected by filtration and washed with CH₃CN to provide compound 1013e (1.78 g, 80.9%).

### Step 5

To a solution of 1013e (1.78 g, 3.13 mmol) in dry toluene (25 mL) was added DDQ (1.07 g, 4.71 mmol). After refluxing for 2 hours, the solvent was removed and diluted with EtOAc. The organic layer was washed with saturated Na₂S₂O₃ solution and brine, dried over Na₂SO₄, filtered and concentrated. The residue was washed with MeOH (50 mL), filtered to provide 1013f (1.5 g, 84.7%).

### Step 6

A suspension of 1013f (1.5 g, 2.65 mmol), bis(pinacolato)diboron (1.68 g, 6.6 mmol), KOAc (1.04 g, 10.6 mmol) and Pd(dppf)Cl₂ (0.19 g, 0.27 mmol) in dioxane (10 mL) was allowed to stir at 100° C. for 2 hours under N₂ protection. The reaction mixture was cooled and concentrated in vacuum, the residue was purification by SiO₂ chromatography, eluting with petroleum ether:ethyl acetate (30:1) to provide 1013g (1.74 g, 99.7%).

### Step 7

A suspension of 1013g (1.74 g, 2.64 mmol), methyl ((S)-1-((S)-2-(5-bromo-1H-imidazol-2-yl)pyrrolidin-1-yl)-3-methyl-1-oxobutan-2-yl)carbamate (2.17 g, 5.81 mmol), Pd(dppf)₂Cl₂ (0.19 g, 0.26 mmol) and Na₂CO₃ (1.12 g, 10.56 mmol) in THF/H₂O (8:1, 45 mL) was allowed to stir at 80° C. for overnight under N₂ protection. After that, the mixture was washed with water and extracted with ethyl acetate, washed with brine and dried over anhydrous sodium sulfate. After concentrated in vacuum, the residue was purified using SiO₂ chromatography, eluting with petroleum ether:ethyl acetate (8/1˜2/1) to provide 1013h (970 mg, 37.3%).

### Step 8

The two diastereoisomers of 1013h (970 mg) were separated by SFC by using the following conditions to provide 843 (220 mg, 22.7%) and 844 (120 mg, 12.4%).

Column: Chiralcel OD-3 150×4.6 mm I.D.

Mobile phase: 40% methanol (0.05% DEA) in CO2 from 5% to 40%

Flow rate: 2.5 mL/min

Wavelength: 270 nm

1012: ¹H NMR (400 MHz, METHANOL-d4) δ: 8.03 (s, 1H), 7.97 (s, 1H), 7.82 (s, 1H), 7.76 (s, 1H), 7.51 (s, 1H), 7.46 (br. s., 1H), 7.41-7.36 (m, 1H), 7.34 (s, 1H), 7.20 (d, J=2.3 Hz, 1H), 6.58 (d, J=3.1 Hz, 1H), 6.51 (d, J=3.1 Hz, 1H), 5.21 (td, J=7.2, 14.2 Hz, 2H), 4.20 (t, J=8.0 Hz, 2H), 4.08 (br. s., 2H), 3.89-3.82 (m, 2H), 3.64 (s, 6H), 3.14 (s, 3H), 2.84 (s, 2H), 2.55 (br. s., 2H), 2.26 (d, J=4.7 Hz, 2H), 2.16 (d, J=3.9 Hz, 4H), 2.03 (td, J=6.6, 13.5 Hz, 2H), 1.05 (s, 6H), 1.00-0.84 (m, 12H). LC/MS: Anal. Calcd. For [M+H]⁺ C52H62FN9O8S; 992.19. found 992.8.

1013: ¹H NMR (400 MHz, METHANOL-d4) δ: 8.03 (s, 1H), 7.98 (s, 1H), 7.83 (s, 1H), 7.77 (s, 1H), 7.55-7.51 (m, 1H), 7.47 (d, J=8.6 Hz, 1H), 7.39 (d, J=10.6 Hz, 1H), 7.34 (s, 1H), 7.22 (d, J=2.7 Hz, 1H), 6.58 (d, J=3.1 Hz, 1H), 6.50 (d, J=3.5 Hz, 1H), 5.22 (m, 2H), 4.21 (t, J=7.8 Hz, 2H), 4.09 (br. s., 2H), 3.90-3.82 (m, 2H), 3.64 (s, 6H), 3.14 (s, 3H), 2.84 (s, 2H), 2.56 (br. s., 2H), 2.26 (d, J=5.1 Hz, 2H), 2.17 (d, J=5.1 Hz, 4H), 2.04 (m, 2H), 1.05 (s, 6H), 1.00-0.84 (m, 12H). LC/MS: Anal. Calcd. For [M+H]⁺ C52H62FN9O8S; 992.19. found 992.6.

The following compounds were prepared according to the procedure described above.

| | | | Observed |
|---|---|---|---|
| ID | Structures | Isomer | [M + H]+ |
| 845 | 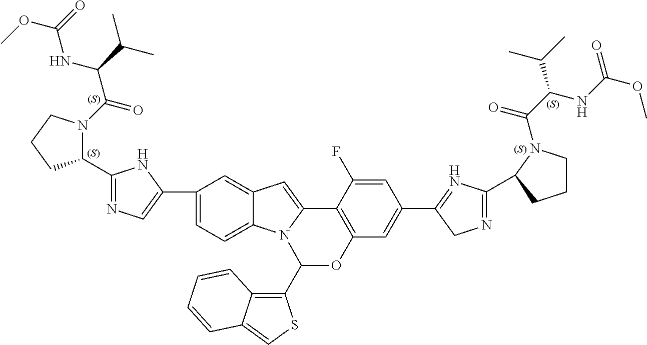 | racemic | 956.8 |
| 846 | 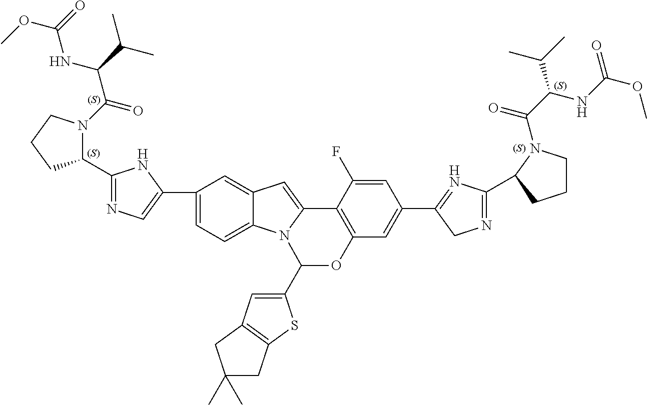 | racemic | 974.6 |
| 847 | 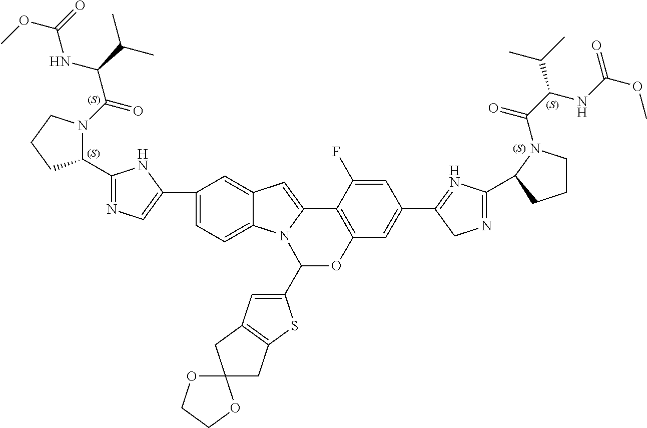 | racemic | 1004.6 |

### Example 96

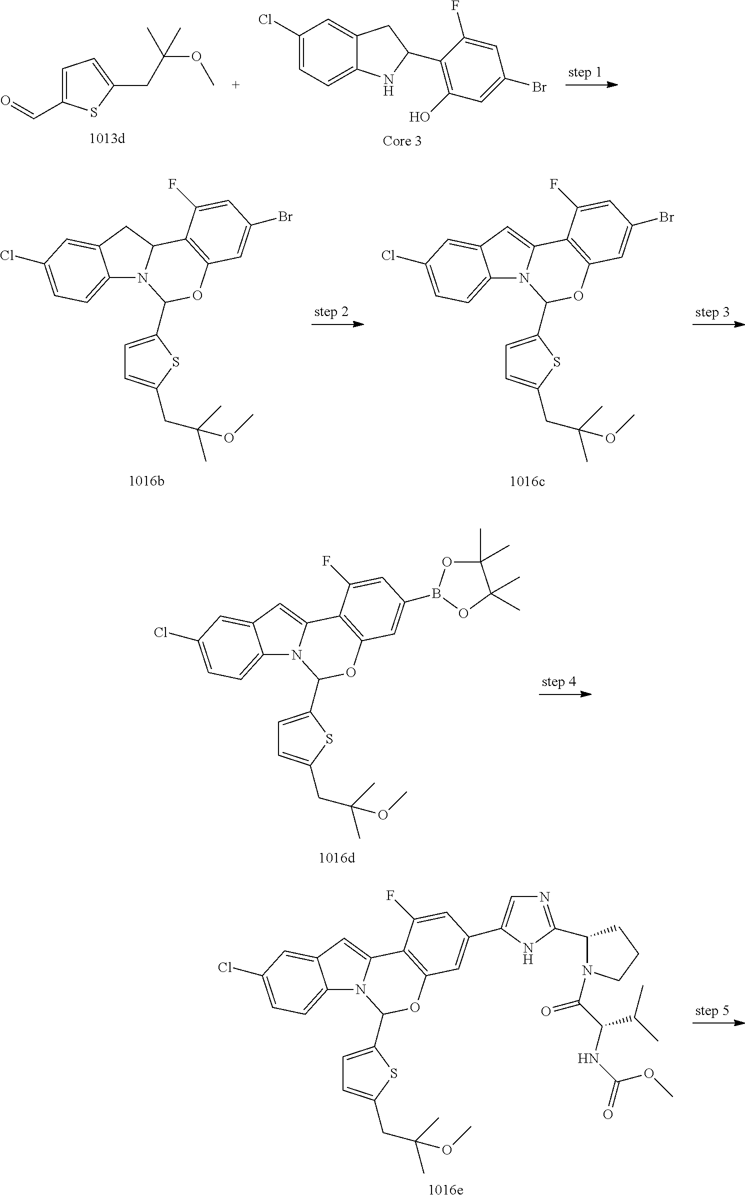 [SMILES: COC(C)(C)Cc1ccc(C=O)s1 ; Oc1cc(Br)cc(F)c1C1Cc2cc(Cl)ccc2N1 ; COC(C)(C)Cc1ccc(C2Oc3cc(Br)cc(F)c3C3Cc4cc(Cl)ccc4N32)s1 ; COC(C)(C)Cc1ccc(C2Oc3cc(Br)cc(F)c3-c3cc4cc(Cl)ccc4n32)s1 ; COC(C)(C)Cc1ccc(C2Oc3cc(B4OC(C)(C)C(C)(C)O4)cc(F)c3-c3cc4cc(Cl)ccc4n32)s1 ; COC(=O)N[C@H](C(=O)N1CCC[C@H]1c1ncc(-c2cc(F)c3c(c2)OC(c2ccc(CC(C)(C)OC)s2)n2c-3cc3cc(Cl)ccc32)[nH]1)C(C)C]

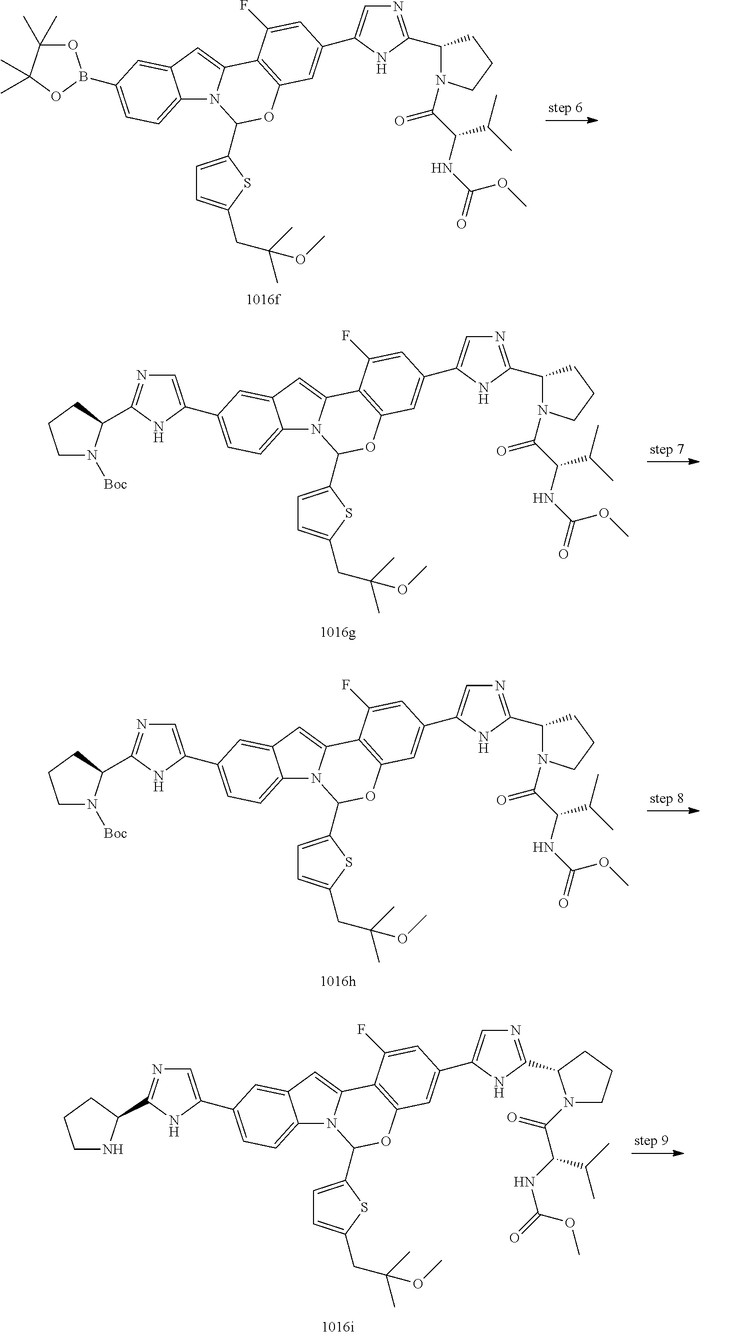

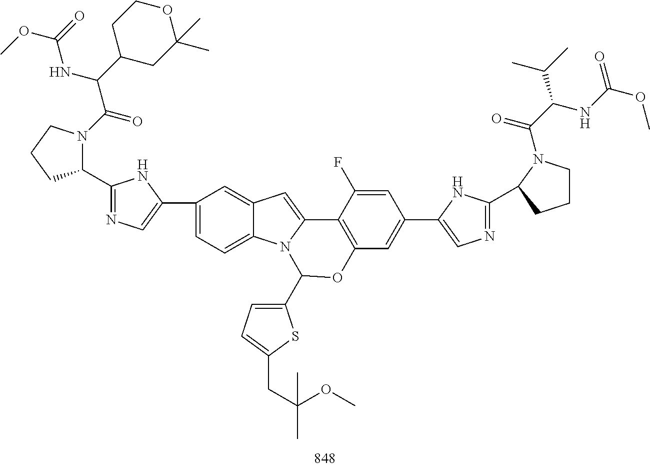

### Step 1

Compound 1013d was prepared in Example 95.

To a mixture of 1013d (2.5 g, 12.63 mmol) and Core3 (3.3 g, 9.71 mmol) in anhydrous CH₃CN (30 mL) was added TFA (330 mg, 2.91 mmol) at room temperature. The mixture was agitated overnight at room temperature. The reaction mixture became a clear solution and then solid appeared. The solid was collected by filtration and washed with CH₃CN to provide 1016b (4.54 g, 89.5%).

### Step 2

The solution of 1016b (4.54 g, 8.68 mmol) in dry toluene (60 mL) was added DDQ (2.96 g, 13.03 mmol). After refluxing for 2h, the solvent was removed and diluted with EtOAc. The organic layer was washed with saturated Na₂S₂O₃ aqueous and brine, dried over Na₂SO₄, filtered and concentrated. The residue was washed with MeOH (50 mL), filtered to provide 1016c (3.3 g, 73%).

### Step 3

To a solution of 1016c (3.3 g, 6.34 mmol) in 1,4-dioxane (40 mL) was added bis(pinacolato)diboron (1.93 g, 7.6 mmol) and Pd(dppf)Cl₂ (0.46 g, 0.63 mmol) and KOAc (1.24 g, 12.68 mmol). The reaction mixture was allowed to stir under N₂ and heated to 100° C. for overnight. After that, the solvent was removed under vacuum, and the residue was purified using SiO₂ chromatography, eluting with petroleum ether: ethyl acetate (5/1˜1/1) to provide 1016d (3.3 g, 91.7%).

### Step 4

A suspension of 1016d (3.3 g, 5.81 mmol), methyl ((S)-1-((S)-2-(5-bromo-1H-Imidazol-2-yl)pyrrolidin-1-yl)-3-methyl-1-oxobutan-2-yl)carbamate (2.6 g, 6.97 mmol), Pd(dppf)₂Cl₂ (0.43 g, 0.58 mmol), Na₂CO₃ (1.23 g, 11.62 mmol) and in THF/H₂O (8:1, 90 mL) was refluxed at 75° C. overnight under N₂ protection. After that, the mixture was filtered, and the filtrate was washed with water (50 mL) and extracted with EA (150 mL), washed with brine and dried over anhydrous sodium sulfate. After concentrated in vacuum, the residue was purified using SiO₂ chromatography, eluting with petroleum ether:ethyl acetate (5/1˜1/1) to provide 1016e (3.25 g, 76.3%).

### Step 5

To a mixture of 1016e (2.5 g, 3.41 mmol), bis(pinacolato)diboron (1.93 g, 7.6 mmol), (0.67 g, 6.82 mmol), Pd₂(dba)₃ (0.31 g, 0.34 mmol), X-phos (0.324 g, 0.68 mmol) degassed and sealed under N₂ was added dry dioxane. Following further N₂ purging. The mixture was allowed to stir at 120° C. overnight. Under standard work-up to provide the residue which was purified using SiO₂ chromatography, eluting with petroleum ether:ethyl acetate (5/1˜1/1) to provide 1016f (2.3 g, 82.1% yield).

### Step 6

A suspension of 1016f (1 g, 1.21 mmol), Cap7a (0.46 g, 1.45 mmol), Pd(dppf)₂Cl₂ (89 mg, 0.12 mmol) and Na₂CO₃ (0.26 g, 2.42 mmol) and in THF/H₂O (8:1, 45 mL) was refluxed at 75° C. overnight under N₂ protection. After that, the mixture was filtered, and the filtrate was washed with water (50 mL) and extracted with EA (100 mL), washed with brine and dried over anhydrous sodium sulfate. After concentrated in vacuum, the residue was purified using SiO₂ chromatography, eluting with DCM:MeOH (5/1˜3/1) to provide 1016g (470 mg, 41.6% yield).

### Step 7

1016g (470 mg) was separated by SFC by using the following conditions to provide 1016h (230 mg, 48.9%).

Column: Chiralcel OZ-3 150×4.6 mm I.D.

Mobile phase: 50% of ethanol (0.05% DEA) in CO2

Flow rate: 2.0 mL/min

Wavelength: 220 nm

### Step 8

1016h (100 mg, 0.107 mmol) was added into HCl/dioxane (10 mL).

Then the mixture was allowed to stir at room temperature for 2-3 hr. When the reaction completed, the mixture was concentrated in vacuum to provide 1016i (89 mg, 100%).

### Step 9

To a mixture of 1016i (89 mg, 0.107 mmol), Cap3 (32 mg, 0.128 mmol) and HATU (50 mg, 0.128 mmol) in DMF (10 mL) was added DIPEA (0.5 mL). The resulting mixture was allowed to stir at room temperature for 16 hours before the solution was subjected directly to HPLC to provide 848. ¹H NMR (400 MHz, METHANOL-d4) δ: 8.05 (s, 1H), 7.98 (s, 1H), 7.83 (s, 1H), 7.80 (s, 1H), 7.53 (s, 1H), 7.48 (s, 1H), 7.40 (s, 1H), 7.34 (s, 1H), 7.21 (d, J=2.8 Hz, 1H), 6.58 (d, J=3.0 Hz, 1H), 6.48 (d, J=3.8 Hz, 1H), 5.25-5.17 (m, 2H), 4.21-4.16 (m, 2H), 4.14-4.05 (m, 1H), 3.88-3.80 (m, 2H), 3.64 (s, 6H), 3.14 (s, 3H), 2.83 (s, 2H), 2.59-2.52 (m, 2H), 2.24 (m, 2H), 2.19-2.10 (m, 4H), 2.07-1.97 (m, 2H), 1.60-1.54 (m, 2H), 1.22-1.19 (m, 2H), 1.05 (m, 12H), 0.88 (dd, J=6.8, 10.5 Hz, 8H). LC/MS: Anal. Calcd. For [M+H]⁺ C56H68FN9O9S; 1062.28. found 1062.6.

The following compounds were prepared according to the procedure described above.

| | | | Observed |
|---|---|---|---|
| ID | Structures | Isomer | [M + H]+ |
| 1849 | 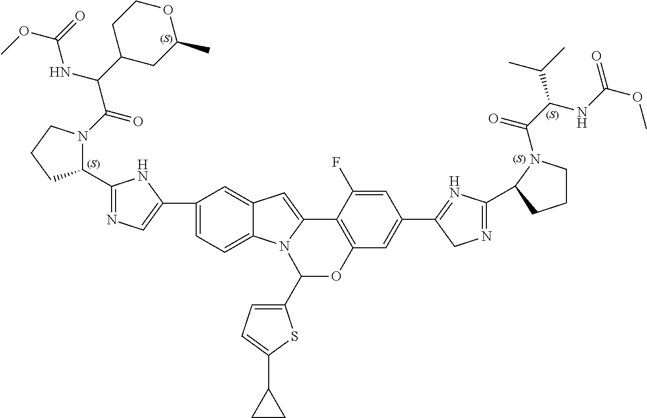 | racemic | 1002.8 |
| 850 | 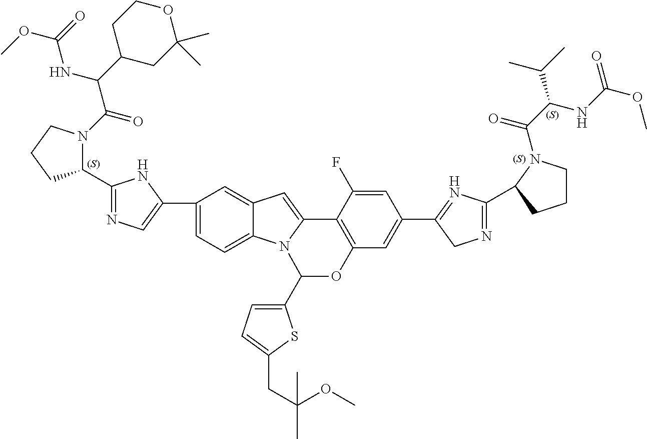 | Isomer 2 | 1062.6 |
| 851 852 853 | 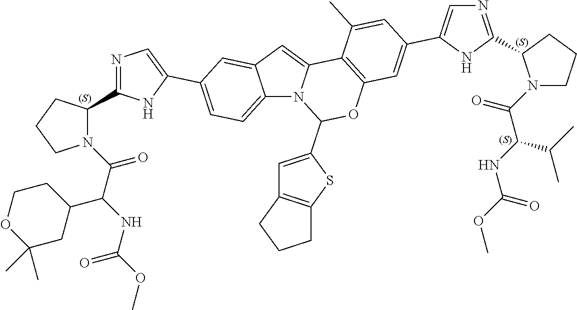 | Racemic Isomer 1 Isomer 2 | 1013.64 1013.3 1013.3 |

### Example 97

### Cell-Based HCV Replicon Assays

To measure cell-based anti-HCV activity of compounds of the present invention, two complimentary assays were employed using various replicons. In the first assay (“Replicon Assay A”), replicon cells were seeded at 2000 cells/well in 384-well 384-well flat bottom tissue culture treated clear bottom plate (Corning 3707) in the presence of the test compound. Various concentrations of test compound, typically in 10 serial dilutions, were added to the assay mixture, with the starting concentration ranging from 333.3 nM to 1.667 nM. The final concentration of DMSO was 0.5%. Fetal bovine serum was 5%, in the assay media. Cells were harvested on day 3 by removing media and washing the cells with a suitable wash buffer. The cells are lysed with the addition of 1× Qiagen lysis buffer (Cat #1062731). The replicon RNA level was measured using real time PCR (TaqMan® EZ RT-PCR, Applied Biosystems 403028) with the following primers and probes:

Neo Forward:
CCG GCT ACC TGC CCA TTC
Neo Reverse:
CCA GAT CAT CCT GAT CGA CAA G
Neo Probe:
FAM-ACA TCG CAT CGA GCG AGC ACG TAC-Tamra
Cyc probe:
5′-JOE-CGCGTCTCCTTTGAGCTGTTTGCA-Tamra-3′
Cyc Forward Primer:
ACGGCGAGCCCTTGG
Cyc Reverse Primer:
TTTCTGCTGTCTTTGGGACCT

Cyclophilin RNA was used as endogenous control and was amplified in the same reaction as NS5B (multiplex PCR). The real-time RT-PCR reactions were run on ABI PRISM 7900HT Sequence Detection System using the following program: 50° C. for 2 minutes, 60° C. for 30 minutes, 95° C. for 5 minutes, 40 cycles of 94° C. for 20 sec, 55° C. for 1 minutes.

The amount of HCV replicon RNA per cell is quantified using a linear regression curve for a known nanogram (ng) amount of HCV replicon total RNA. This is established by plotting the Cycle Threshold values (Ct) from the Neo probe and primer set versus the log (ng) for each HCV replicon total RNA standard. The amount of HCV RNA for each replicon sample is calculated by taking the sample's Ct value, minus the line intercept, divided by the slope of the line. Similarly, the amount of Cyclophilin mRNA per cell is also quantified using a linear regression curve for a known nanogram (ng) amount of HCV replicon total RNA. Again, this is established by plotting the Cycle Threshold values (Ct) from the Cyclophilin probe and primer set versus the log (ng) for each HCV replicon total RNA standard.

In an alternate assay (“Replicon Assay B”), 1000 cells were seeded per well in a 384-well collagen coated black plate from Greiner bio-one (Cat #781946) in 5% FBS. Inhibitors of this invention were added at 24 h post-seeding, and the plates were incubated for 3 days. Cells were subsequently lysed with Qiagen lysis buffer (Cat #1062731) to extract the RNA. HCV replicon RNA level was measured by real-time PCR using the RNA-to-CT kit from Applied Biosystem (Cat #4392656) and genotype-specific primers and probes. The amplicon was located within NS5B. The sequence of the PCR primers are as follows: 5B.2F, ATGGACAGGCGCCCTGA (SEQ. ID NO. 1); 5B.2R, TTGATGGGCAGCTTGGTTTC (SEQ. ID NO. 2); the probe sequence was FAM-labeled CACGCCATGCGCTGCGG (SEQ. ID NO. 3). To detect genotype 1A the primer 1A F, TGCGGAACCGGTGAGTACA and 1A R, GCGGGTTTATCCAAGAAAGGA were used; the probe sequence was FAM-CGGAATTGCCAGGACGACCGG.

The real-time RT-PCR reactions were run on ABI PRISM 7900HT or Viia7 Sequence Detection System using the following program: 48° C. for 30 minutes, 95° C. for 10 minutes, 40 cycles of 95° C. for 15 sec, 60° C. for 1 minutes. The 50% effective concentration (EC₅₀) was the drug concentration necessary to achieve an increase in the cycle threshold (C_{T}) of 1 over the projected baseline C_{T}. The EC₉₀ was the drug concentration necessary to achieve an increase in C_{T} of 3.2 over the projected baseline C_{T}.

Data was obtained for various compounds of the present invention using the methods described in the Example above, and is presented in the table immediately below. Data for replicons 1A, 1AY93H and 2B were obtained using Replicon Assay A and data for replicons 1AQ30D and 1B were obtained using Replicon Assay B.

| | | 1A | 1A | | |
|---|---|---|---|---|---|
| | 1A | Y93H | Q30D | 1B | 2B |
| | IC50 | IC50 | IC50 | IC50 | IC50 |
| ID | (nM) | (nM) | (nM) | (nM) | (nM) |
| 2 | 0.0029 | 0.032 | 0.10 | 0.0024 | 0.033 |
| 3 | 0.0036 | 4.70 | 50.59 | 0.0012 | 4.45 |
| 4 | 0.0022 | 0.010 | 0.347 | 0.0020 | 0.055 |
| 5 | 0.0046 | 0.95 | 28.28 | | 2.081 |
| 6 | 0.0019 | 0.011 | 0.32 | 0.0015 | 0.028 |
| 7 | 0.0045 | 1.78 | 70.74 | 0.0031 | 3.20 |
| 8 | 0.0174 | 0.33 | 0.47 | 0.0027 | 0.14 |
| 9 | 0.0028 | 0.039 | 1.42 | 0.0016 | 0.10 |
| 10 | 0.0017 | 0.004 | 0.058 | 0.0024 | 0.028 |
| 11 | 0.0017 | 7.87 | | | 2.02 |
| 12 | 0.0020 | 0.049 | 0.69 | 0.0027 | 0.057 |
| 13 | 0.0019 | 2.10 | | 0.0023 | 3.79 |
| 14 | 0.0020 | 0.059 | | 0.0024 | 0.23 |
| 15 | 0.0029 | 18.12 | | | 2.28 |
| 16 | 0.0024 | 0.49 | | | 0.171 |
| 17 | 0.0026 | 0.19 | 2.91 | 0.0021 | 0.54 |
| 18 | 0.0022 | 3.13 | 66.73 | 0.0030 | 3.46 |
| 19 | 0.0016 | 0.50 | | | 1.88 |
| 20 | 0.0009 | 0.014 | 0.38 | 0.0011 | 0.051 |
| 21 | 0.0019 | 0.22 | | | 0.26 |
| 22 | 0.0020 | 1.38 | | | 1.84 |
| 23 | 0.0032 | 0.24 | 1.75 | 0.0039 | 0.78 |
| 24 | 0.0027 | 1.35 | 14.91 | 0.0031 | 2.75 |
| 25 | 0.0043 | 14.36 | | | 15.05 |
| 27 | 0.0016 | 0.65 | | 0.0014 | 1.49 |
| 28 | 0.0011 | 0.008 | 0.69 | 0.0019 | 0.086 |
| 29 | 0.0024 | 1.32 | | | 1.98 |
| 30 | 0.0513 | | | | 33.33 |
| 31 | 0.0020 | 0.020 | | | 0.083 |
| 32 | 0.0013 | 1.42 | | | 6.02 |
| 33 | 0.0018 | 0.28 | | | 1.89 |
| 34 | 0.0028 | 0.005 | 0.16 | 0.0009 | 0.10 |
| 35 | 0.0017 | 0.62 | 11.54 | 0.0014 | 1.43 |
| 36 | 0.0017 | 0.006 | 0.19 | 0.0011 | 0.053 |
| 39 | 0.0020 | 0.12 | 0.61 | 0.0010 | 0.45 |
| 40 | 0.0020 | 1.12 | | | |
| 41 | 0.0020 | 2.45 | | | |
| 42 | 0.0287 | 42.47 | | | |
| 43 | 0.0019 | 0.12 | | | 0.54 |
| 44 | 0.0015 | 0.012 | 0.54 | 0.0012 | 0.10 |
| 45 | 0.0011 | 0.010 | 0.37 | 0.0011 | 0.044 |
| 46 | 0.0029 | 2.60 | 17.08 | 0.0010 | 1.79 |
| 47 | 0.0016 | 1.54 | | | 0.77 |
| 48 | 0.0019 | 0.027 | 0.54 | 0.0017 | 0.016 |
| 49 | 0.0013 | 0.002 | 0.16 | 0.0012 | 0.19 |
| 50 | 0.0014 | 0.0284 | | | 1.385 |
| 51 | 0.0047 | 5.28 | 33.33 | 0.0023 | 4.51 |
| 52 | 0.0030 | 0.07 | 0.81 | 0.0023 | 0.21 |
| 53 | 0.0009 | 1.87 | | | 1.95 |
| 54 | 0.0020 | 0.005 | 0.20 | 0.0012 | 0.030 |
| 55 | 0.0019 | 3.26 | | | 2.53 |
| 56 | 0.0032 | 0.048 | 0.54 | 0.0015 | 0.12 |
| 57 | 0.0114 | 0.37 | | | 2.79 |
| 58 | 0.0014 | 0.002 | 0.041 | 0.0034 | 0.011 |
| 59 | 0.0013 | 0.004 | 0.018 | 0.0035 | 0.007 |
| 60 | 0.0015 | 0.014 | 0.35 | 0.0013 | 0.023 |
| 61 | 0.0011 | 0.014 | 0.22 | 0.0011 | 0.0120 |
| 62 | 0.0014 | 0.69 | | | 2.88 |
| 63 | 0.0032 | 0.010 | 0.34 | 0.0024 | 0.27 |
| 64 | 0.0040 | 0.15 | | | 4.008 |
| 65 | 0.0016 | 3.00 | | | 3.60 |
| 66 | 0.0009 | 0.13 | | 0.0009 | 0.23 |
| 67 | 0.0009 | 0.015 | | 0.0009 | 0.029 |
| 68 | 0.0008 | 0.12 | | | 1.12 |
| 70 | 0.0188 | 10.51 | | | 24.17 |
| 71 | 0.0013 | 1.087 | | | 3.85 |
| 72 | 0.0026 | 0.010 | 0.78 | 0.0015 | 0.089 |
| 73 | 0.0016 | 2.26 | | | 5.14 |
| 74 | 0.0012 | 0.031 | 0.39 | 0.0015 | 0.056 |
| 75 | 0.0030 | 0.021 | 0.27 | 0.0031 | 0.041 |
| 76 | 0.0159 | 2.55 | | | 13.66 |
| 77 | 0.0018 | 0.24 | | 0.0029 | 0.41 |
| 79 | 0.0021 | 0.14 | | 0.0034 | 0.32 |
| 80 | 0.0027 | 4.87 | | | 3.16 |
| 81 | 0.0014 | 0.41 | | | 1.32 |
| 82 | 0.0011 | 0.014 | 0.26 | 0.0013 | 0.15 |
| 83 | 0.0032 | 0.42 | | 0.0031 | 0.80 |
| 84 | 0.0033 | 0.012 | 0.50 | 0.0025 | 0.012 |
| 85 | 0.0011 | 0.41 | | | 1.00 |
| 86 | 0.0016 | 0.003 | 0.11 | 0.0016 | 0.033 |
| 87 | 0.0012 | 0.002 | 0.15 | 0.0013 | 0.19 |
| 88 | 0.0008 | 0.007 | 0.30 | 0.0014 | 0.59 |
| 89 | 0.0010 | 0.033 | 0.33 | 0.0015 | 0.063 |
| 90 | 0.0007 | 0.74 | | | 1.91 |
| 91 | 0.0008 | 0.28 | | | 3.14 |
| 92 | 0.0014 | 0.008 | 0.11 | 0.0009 | 0.076 |
| 93 | 0.0030 | 0.007 | 0.053 | 0.0025 | 0.054 |
| 94 | 0.0012 | 0.39 | 2.56 | 0.0018 | 1.97 |
| 95 | 0.0010 | 0.71 | | | |
| 96 | 0.0132 | 3.31 | | | |
| 97 | 0.0011 | 0.008 | | 0.0024 | 0.036 |
| 98 | 0.0011 | 0.060 | | | 2.16 |
| 99 | 0.0009 | 0.53 | | | 1.55 |
| 100 | 0.0242 | 8.26 | | | |
| 101 | 0.0021 | 0.020 | 0.54 | 0.0014 | 0.028 |
| 102 | 0.0011 | 4.37 | 45.74 | 0.0017 | 1.42 |
| 103 | 0.0056 | 5.15 | | | 1.71 |
| 104 | 0.0043 | 0.046 | 1.11 | 0.0051 | 0.073 |
| 105 | 0.0015 | 0.010 | 0.030 | 0.0029 | 0.012 |
| 106 | 0.0021 | 0.16 | 0.030 | 0.0015 | 0.35 |
| 107 | 0.0024 | 0.014 | 0.044 | 0.0033 | 0.017 |
| 108 | 0.0026 | 0.25 | 1.15 | 0.0027 | 0.29 |
| 109 | 0.0058 | 4.50 | | | 24.35 |
| 110 | 0.0020 | 1.97 | | | 2.43 |
| 111 | 0.0032 | 0.074 | 0.13 | 0.0017 | 0.038 |
| 112 | 0.0022 | 0.23 | | | 7.07 |
| 113 | 0.0428 | 3.92 | | | |
| 114 | 0.0013 | 0.002 | | 0.0024 | 0.20 |
| 115 | 0.0011 | 0.050 | | | 9.84 |
| 116 | 0.0009 | | | | 1.85 |
| 117 | 0.0009 | | | | 0.028 |
| 118 | 0.0012 | | | | 0.97 |
| 119 | 0.0008 | | | | 0.073 |
| 120 | 0.0015 | 0.1 | | | 1.44 |
| 121 | 0.0012 | | | | 0.17 |
| 122 | 0.0020 | 0.007 | 0.029 | 0.0023 | 0.003 |
| 123 | 0.0012 | 0.59 | 0.38 | 0.0031 | 0.064 |
| 124 | 0.0018 | 1.38 | | | 3.40 |
| 125 | 0.0023 | 0.018 | 0.29 | 0.0013 | 0.089 |
| 126 | 0.0019 | 0.27 | | | 0.21 |
| 127 | 0.0020 | 0.002 | 0.020 | 0.0028 | 0.003 |
| 128 | 0.0030 | 0.030 | 0.044 | 0.0036 | 0.045 |
| 129 | 0.0010 | 0.80 | | | 1.17 |
| 130 | 0.0017 | 0.24 | | 0.0036 | 0.24 |
| 131 | 0.0078 | 3.83 | | | 9.78 |
| 132 | 0.0032 | 4.93 | | | 6.001 |
| 133 | 0.0020 | | | | 0.15 |
| 134 | 0.0025 | 0.002 | 0.015 | 0.0026 | 0.003 |
| 135 | 0.0024 | 0.004 | 0.027 | 0.0030 | 0.006 |
| 136 | 0.0018 | 0.20 | 0.90 | 0.0017 | 0.23 |
| 137 | 0.0018 | 0.28 | | | 0.37 |
| 138 | 0.0013 | 0.019 | 0.67 | 0.0014 | 0.061 |
| 139 | 0.0019 | 0.085 | 1.03 | 0.0014 | 0.11 |
| 140 | 0.0407 | 5.51 | | | 8.69 |
| 141 | 0.0584 | 12.83 | | | 21.37 |
| 142 | 0.0020 | 0.25 | | | 0.48 |
| 143 | 0.1112 | | | 0.0059 | |
| 144 | 0.0030 | 0.003 | 0.057 | 0.0021 | 0.022 |
| 145 | 0.0022 | 5.22 | | | 1.62 |
| 146 | 0.0012 | 0.57 | | | 0.38 |
| 147 | 0.0025 | 0.43 | | | 0.62 |
| 148 | 0.0919 | 10.16 | | | 13.17 |
| 149 | 0.1294 | 25.85 | | | 24.78 |
| 150 | 0.0024 | 0.004 | 0.094 | 0.0019 | 0.007 |
| 151 | 0.0020 | 0.003 | 0.022 | 0.0024 | 0.004 |
| 152 | 0.0016 | 0.48 | | | 0.53 |
| 153 | 0.0021 | 0.422 | | | 0.51 |
| 154 | 0.0017 | 0.36 | | | 0.55 |
| 155 | 0.167 | | | | |
| 156 | 0.0020 | 0.005 | 0.019 | 0.0018 | 0.005 |
| 157 | 0.0021 | 2.32 | | | 2.80 |
| 158 | 0.0033 | 1.07 | | | 0.43 |
| 159 | 0.0025 | 0.77 | | | 0.45 |
| 160 | 0.1142 | 24.95 | | | 19.05 |
| 161 | 0.0586 | | | | |
| 162 | 0.0022 | 0.004 | 0.027 | 0.0019 | 0.006 |
| 163 | 0.0184 | 0.058 | 0.17 | 0.0313 | 0.063 |
| 164 | 0.0015 | 0.13 | | | 0.53 |
| 165 | 0.0020 | 0.27 | | | 0.24 |
| 166 | 0.0019 | | | | 0.072 |
| 167 | 0.0008 | | | | 0.320 |
| 168 | 0.0013 | 0.002 | 0.011 | 0.0015 | 0.002 |
| 169 | 0.0012 | 0.045 | | 0.0034 | 0.075 |
| 170 | 0.0010 | 0.056 | | 0.0033 | 0.050 |
| 171 | 0.0131 | 1.40 | | | 4.056 |
| 172 | 0.0015 | 0.004 | 0.018 | 0.0018 | 0.005 |
| 173 | 0.0017 | 0.098 | 0.28 | 0.0021 | 0.35 |
| 174 | 0.0011 | 0.614 | | | 0.16 |
| 175 | 0.0077 | 9.027 | | | 10.54 |
| 176 | 0.0011 | | | | 0.046 |
| 177 | 0.0014 | | | | 0.016 |
| 178 | 0.0017 | | | | 3.40 |
| 179 | 0.0012 | | | | 4.63 |
| 180 | 0.0025 | | | | |
| 181 | 0.0023 | 0.054 | | | 0.083 |
| 182 | 0.0020 | 0.044 | 0.049 | 0.0020 | 0.005 |
| 183 | 0.0012 | 0.74 | 1.08 | 0.0024 | 0.68 |
| 184 | 0.0020 | 0.16 | 0.21 | 0.0020 | 0.076 |
| 185 | 0.0046 | 2.40 | 2.95 | 0.0026 | 1.35 |
| 186 | 0.0019 | 0.53 | 2.84 | 0.0021 | 0.63 |
| 187 | 0.0664 | 14.80 | | | |
| 188 | 0.0017 | 0.002 | 0.020 | 0.0012 | 0.005 |
| 189 | 0.0013 | 0.26 | 1.42 | 0.0018 | 0.48 |
| 190 | 0.0011 | 0.002 | 0.013 | 0.0014 | 0.003 |
| 191 | 0.0014 | 0.32 | | | 0.89 |
| 192 | 0.0011 | 0.028 | | | 0.047 |
| 193 | 0.0256 | 4.75 | | | 18.55 |
| 194 | 0.0019 | 0.095 | | | 0.63 |
| 195 | 0.0026 | 0.002 | 0.030 | 0.0034 | 0.016 |
| 196 | 0.0018 | 0.062 | | | 0.55 |
| 197 | 0.0021 | 0.10 | | | 0.47 |
| 198 | 0.0770 | 14.33 | | | |
| 199 | 0.1178 | 26.52 | | | 33.33 |
| 200 | 0.0021 | 0.002 | 0.036 | 0.0030 | 0.014 |
| 201 | 0.0015 | 0.002 | 0.015 | 0.0022 | 0.008 |
| 202 | 0.0013 | 0.49 | | | 3.31 |
| 203 | 0.0016 | 0.12 | | | 0.83 |
| 204 | 0.0008 | | | | 0.26 |
| 205 | 0.0009 | | | | |
| 206 | 0.0014 | 0.16 | 2.91 | 0.0026 | 0.16 |
| 208 | 0.0031 | 0.008 | 0.043 | 0.0032 | 0.005 |
| 210 | 0.0018 | 0.52 | | | 0.33 |
| 211 | 0.0026 | 0.22 | | | 0.42 |
| 212 | 0.0681 | 6.07 | | | 8.59 |
| 213 | 0.0653 | 13.57 | | | |
| 214 | 0.0030 | 0.009 | 0.032 | 0.0022 | 0.005 |
| 215 | 0.0031 | 0.005 | 0.017 | 0.0032 | 0.010 |
| 216 | 0.0017 | 0.18 | 1.030 | 0.0033 | 0.31 |
| 217 | 0.0030 | 0.18 | | | 0.39 |
| 218 | 0.0026 | 0.004 | 0.055 | 0.0031 | 0.004 |
| 219 | 0.0022 | 0.003 | 0.034 | 0.0032 | 0.005 |
| 220 | 0.0021 | 0.52 | | | 0.93 |
| 221 | 0.0022 | 0.18 | | | 0.21 |
| 222 | 0.0020 | 0.24 | | | 0.17 |
| 223 | 0.0025 | 0.24 | | | 0.27 |
| 224 | 0.0480 | 8.88 | | | |
| 225 | 0.0477 | | | 0.0102 | 13.71 |
| 226 | 0.0026 | 0.009 | 0.13 | 0.0036 | 0.025 |
| 227 | 0.0019 | 0.15 | | | 0.17 |
| 228 | 0.002 | 0.50 | | 0.0038 | 0.47 |
| 230 | 0.0184 | | | | |
| 231 | 0.0016 | 2.05 | | | |
| 232 | 0.0027 | 0.20 | | | 0.63 |
| 233 | 0.0660 | | | | |
| 234 | 0.0033 | 0.004 | 0.05 | 0.0028 | 0.020 |
| 235 | 0.0013 | 3.87 | | | 10.24 |
| 236 | 0.0017 | 0.18 | | | 0.24 |
| 237 | 0.0012 | 0.21 | | | 0.40 |
| 238 | 0.13 | 3.12 | | | 7.05 |
| 239 | 0.0982 | 6.60 | | | 9.48 |
| 240 | 0.0021 | 0.005 | 0.062 | 0.0027 | 0.011 |
| 241 | 0.0016 | 0.005 | 0.033 | 0.0024 | 0.016 |
| 242 | 0.0011 | 0.75 | | | 4.42 |
| 243 | 0.0015 | 0.87 | | | 6.81 |
| 244 | 0.0013 | 0.30 | | | 0.60 |
| 245 | 0.0022 | 0.003 | 0.021 | 0.0013 | 0.010 |
| 246 | 0.0011 | 0.58 | | | 0.27 |
| 247 | 0.0343 | 0.15 | | | 12.45 |
| 248 | 0.0025 | 0.11 | 0.43 | 0.0032 | 0.076 |
| 249 | 0.0228 | 1.18 | | | 5.04 |
| 250 | 0.0022 | 0.003 | 0.013 | 0.0029 | 0.001 |
| 251 | 0.0012 | 0.060 | 0.40 | 0.0025 | 0.10 |
| 252 | 0.0021 | 0.095 | | | 3.56 |
| 253 | 0.0028 | 0.003 | 0.59 | 0.0034 | 0.32 |
| 254 | 0.0011 | 0.12 | | | 3.49 |
| 255 | 0.0008 | 0.084 | | | 3.63 |
| 256 | 0.117 | 16.85 | | | |
| 257 | 0.0924 | 9.38 | | | |
| 258 | 0.0018 | 0.004 | 1.20 | 0.0027 | 0.43 |
| 259 | 0.0031 | 0.002 | 0.31 | 0.0033 | 0.32 |
| 260 | 0.0010 | 0.047 | | | 11.71 |
| 261 | 0.0015 | 0.043 | | | 9.84 |
| 262 | 0.0042 | 0.052 | 0.12 | 0.004 | 0.019 |
| 263 | 0.0036 | 0.003 | 0.012 | 0.0034 | 0.005 |
| 264 | 0.0037 | 0.029 | 0.70 | 0.0043 | 0.19 |
| 265 | 0.0009 | 0.16 | | | 0.25 |
| 266 | 0.0011 | 0.003 | 0.051 | 0.0015 | 0.008 |
| 267 | 0.0035 | 0.013 | 0.080 | 0.0039 | 0.012 |
| 268 | 0.045 | | | | |
| 269 | 0.0026 | 0.28 | | | 0.25 |
| 270 | 0.62 | | | | |
| 271 | 0.0034 | 0.004 | 0.036 | 0.0022 | 0.013 |
| 272 | 0.0027 | 1.000 | 2.39 | 0.0033 | 2.75 |
| 273 | 0.0015 | 0.18 | 2.06 | 0.0020 | 0.74 |
| 274 | 0.022 | 7.87 | | | |
| 275 | 0.0019 | 0.003 | 0.023 | 0.0032 | 0.006 |
| 276 | 0.0015 | 0.001 | 0.018 | 0.0024 | 0.005 |
| 277 | 0.0009 | 0.13 | | | 0.58 |
| 278 | 0.0018 | 0.161 | | | 0.30 |
| 279 | 0.0023 | 0.21 | 0.92 | 0.0029 | 0.017 |
| 280 | 0.0021 | 0.003 | 0.005 | 0.0026 | 0.003 |
| 281 | 0.0018 | 0.30 | | | 0.19 |
| 282 | 0.0018 | 0.011 | 0.04 | 0.0028 | 0.008 |
| 283 | 0.0009 | 0.192 | | | 0.68 |
| 284 | 0.0018 | 0.002 | 0.021 | 0.0021 | 0.024 |
| 285 | 0.0009 | | | | 0.79 |
| 286 | 0.0010 | | | | 0.018 |
| 287 | 0.0082 | 0.074 | | | 0.49 |
| 288 | 0.0046 | 15.97 | | | |
| 289 | 0.0031 | 1.24 | | 0.0042 | 5.64 |
| 290 | 0.0011 | 0.19 | | | 0.34 |
| 291 | 0.0024 | 0.28 | | 0.0023 | 0.028 |
| 292 | 0.0027 | 0.044 | | | 0.106 |
| 293 | >1.67 | | | | |
| 294 | >1.67 | | | | |
| 295 | 0.0032 | 0.005 | 0.017 | 0.0028 | 0.004 |
| 296 | 0.0028 | 0.002 | 0.005 | 0.0033 | 0.005 |
| 297 | 0.0889 | 8.37 | | | 6.68 |
| 298 | 0.0784 | 1.22 | | | 2.09 |
| 299 | 0.0017 | 0.27 | | | 0.14 |
| 300 | 0.0024 | 0.002 | 0.006 | 0.0032 | 0.003 |
| 301 | 0.0025 | 0.11 | 0.33 | 0.0020 | 0.037 |
| 302 | >13 | | | | |
| 303 | 0.0026 | 0.002 | 0.008 | 0.0013 | 0.003 |
| 304 | 0.0455 | 16.81 | | | |
| 305 | 0.0019 | 0.28 | | | 0.031 |
| 306 | 0.0020 | 0.087 | 0.27 | 0.0031 | 0.033 |
| 307 | | 31.7 | | | 16.7 |
| 308 | 0.0019 | 0.002 | 0.005 | 0.0026 | 0.001 |
| 309 | 0.0028 | 0.001 | 0.007 | 0.0037 | 0.004 |
| 310 | 0.0595 | 0.95 | | | 2.70 |
| 311 | 0.7 | >333 | | | |
| 312 | 0.0089 | 24.50 | | | |
| 313 | 0.0032 | 0.026 | 0.19 | 0.0029 | 0.10 |
| 314 | 0.0065 | 1.12 | | | 2.47 |
| 315 | 0.0094 | 0.21 | 0.29 | 0.0106 | 0.027 |
| 316 | 0.0094 | 0.022 | 0.25 | 0.0124 | 0.050 |
| 317 | 0.0012 | 0.11 | 2.58 | 0.0016 | 0.94 |
| 318 | 0.0019 | 5.90 | | | 6.87 |
| 319 | 0.0010 | 9.38 | | | 8.0590 |
| 320 | 0.0011 | 5.50 | | | 5.0980 |
| 321 | 0.0013 | 5.79 | | | |
| 322 | 0.0008 | 0.0297 | 0.82 | 0.0008 | 0.29 |
| 323 | 0.0021 | 2.51 | | | 15.77 |
| 324 | 0.0014 | 4.89 | | | 7.99 |
| 326 | 0.0011 | 0.0110 | 0.1014 | 0.0011 | 0.0663 |
| 327 | 0.0030 | 0.7314 | | | 1.391 |
| 328 | 0.0010 | 2.801 | | | 1.729 |
| 329 | 0.0012 | 0.9373 | | | 0.6045 |
| 330 | 0.0008 | 0.6161 | | | 0.3946 |
| 331 | 0.0012 | 0.0031 | 0.0673 | 0.0017 | 0.0067 |
| 332 | 0.0011 | 0.7652 | | | 1.394 |
| 333 | 0.0010 | | | | 0.0314 |
| 334 | 0.0024 | 0.1587 | | | 0.0768 |
| 335 | 0.0025 | 0.0052 | 0.0639 | 0.0012 | 0.0040 |
| 336 | 0.0022 | 0.0620 | 1.505 | 0.0011 | 0.1275 |
| 337 | 0.0016 | 0.0021 | 0.0266 | 0.0016 | 0.0039 |
| 338 | 0.0024 | 0.3567 | | | 1.525 |
| 339 | 0.0020 | 0.0047 | 0.1332 | 0.0022 | 0.0292 |
| 340 | 0.0011 | 0.0207 | 0.2001 | 0.0025 | 0.0212 |
| 341 | 0.0009 | 0.5199 | | | 1.235 |
| 342 | 0.0016 | 0.0690 | 1.073 | 0.0026 | 0.1658 |
| 343 | 0.0010 | 0.3703 | 0.7041 | 0.0023 | 0.8293 |
| 344 | 0.0015 | 0.1276 | | 0.0009 | 0.2424 |
| 345 | 0.0009 | 0.0012 | 0.0235 | 0.0009 | 0.0072 |
| 346 | 0.0010 | 0.0414 | 0.223 | 0.0013 | 0.0374 |
| 347 | 0.0019 | 0.0023 | 0.0129 | 0.0026 | 0.0028 |
| 348 | 0.0019 | 0.0149 | 0.3873 | 0.0027 | 0.0326 |
| 349 | 0.0014 | 0.2663 | | | 0.3898 |
| 350 | 0.0020 | 0.0023 | 0.0263 | 0.0036 | 0.0027 |
| 351 | 0.0012 | 0.1603 | | | 0.0705 |
| 352 | 0.0064 | 0.0126 | 0.1289 | 0.0085 | 0.0080 |
| 353 | 0.0025 | 0.4185 | | | 0.1463 |
| 354 | 0.0020 | 0.3799 | | | 0.6019 |
| 355 | 0.0019 | 1.749 | | | 2.797 |
| 356 | 0.0025 | 0.0214 | 0.1519 | 0.0025 | 0.0175 |
| 357 | 0.0021 | 0.0041 | 0.0165 | 0.0030 | 0.0026 |
| 358 | 0.0047 | 2.616 | | | 2.493 |
| 359 | 0.0036 | 0.0826 | 0.8481 | 0.0019 | 0.0519 |
| 360 | 0.0025 | 3.564 | | | 2.521 |
| 361 | 0.0036 | 0.1377 | | 0.0035 | 0.0389 |
| 362 | 0.0011 | 0.0775 | 0.9808 | 0.0018 | 0.0486 |
| 363 | 0.0015 | 0.0048 | 0.0439 | 0.0021 | 0.0037 |
| 364 | 0.0020 | 0.0869 | 0.4977 | 0.0023 | 0.0872 |
| 365 | 0.0027 | 0.3803 | | | 0.154 |
| 366 | 0.0019 | 0.0123 | 0.0759 | 0.0015 | 0.0053 |
| 367 | 0.0021 | 0.0343 | 0.361 | 0.0034 | 0.0223 |
| 368 | 0.0026 | 0.0047 | 0.0304 | 0.0031 | 0.0028 |
| 369 | 0.0010 | 0.0739 | 0.912 | 0.0018 | 0.1152 |
| 370 | 0.0014 | 0.0022 | 0.0454 | 0.0020 | 0.0066 |
| 371 | 0.0042 | 0.5527 | | | 0.0627 |
| 372 | 0.0022 | 0.0051 | 0.0964 | 0.0032 | 0.0064 |
| 373 | 0.0010 | 0.2477 | | | 0.1562 |
| 374 | 0.0015 | 0.0035 | 0.0221 | 0.0022 | 0.0041 |
| 375 | 0.0015 | 0.0269 | 0.3148 | 0.0026 | 0.0240 |
| 376 | 0.0026 | 0.0023 | 0.0424 | 0.0029 | 0.0060 |
| 377 | 0.0029 | 0.41 | | | 0.21 |
| 378 | 0.0035 | 0.038 | 0.43 | 0.0025 | 0.097 |
| 379 | 0.0022 | 0.0803 | 0.7984 | 0.0011 | 0.178 |
| 380 | 0.0030 | 0.0052 | 0.0220 | 0.0024 | 0.0056 |
| 381 | 0.0014 | 0.1411 | | | 0.1807 |
| 382 | 0.0017 | 0.0034 | 0.0229 | 0.0025 | 0.0080 |
| 383 | 0.0367 | 6.337 | | | 8.736 |
| 384 | 0.0156 | 0.9232 | 16.67 | 0.0095 | 3.751 |
| 385 | 0.1094 | 3.107 | | | 7.209 |
| 386 | | 5.538 | | | 10.26 |
| 387 | 0.021 | 11.6 | | | 3.7 |
| 388 | | 10.2 | | | 13.6 |
| 389 | 0.0027 | 0.0137 | 0.2296 | 0.0039 | 0.0691 |
| 390 | 0.0030 | 0.3139 | | | 0.7541 |
| 391 | 0.0035 | | | | |
| 392 | 0.0038 | 0.0514 | | | 0.3726 |
| 393 | 0.0019 | 0.2927 | | | 0.2813 |
| 394 | 0.0025 | 0.0053 | 0.1544 | 0.0031 | 0.0106 |
| 395 | 0.0020 | 0.1162 | | | 0.2005 |
| 396 | 0.0025 | 0.0048 | | | 0.0129 |
| 397 | 0.0030 | 0.0040 | 0.0765 | 0.0030 | 0.0164 |
| 398 | 0.0015 | 0.9553 | | | 0.4936 |
| 399 | 0.0027 | 0.0145 | 0.2916 | 0.0008 | 0.2741 |
| 400 | 0.0017 | 0.1513 | | | 1.687 |
| 401 | 0.0018 | | | | 0.0064 |
| 402 | 0.0034 | | | | 0.0054 |
| 403 | 0.0025 | 0.0067 | 0.0703 | 0.0033 | 0.0070 |
| 404 | 0.0022 | 1.247 | | | 1.667 |
| 405 | 0.0023 | 0.0133 | | | 0.0042 |
| 406 | 0.0023 | 0.0041 | 0.0824 | 0.0028 | 0.0161 |
| 407 | 0.0017 | 0.2582 | | 0.0019 | 0.2602 |
| 408 | 0.0035 | 0.0040 | 0.0261 | 0.0019 | 0.0050 |
| 409 | 0.0017 | 0.7456 | | 0.0025 | 0.1808 |
| 410 | 0.0009 | 0.0034 | 0.1058 | 0.0033 | 0.0041 |
| 411 | 0.001 | 0.78 | | | 0.20 |
| 412 | 0.0094 | 0.008 | | | 0.005 |
| 413 | 0.0030 | 0.0141 | 0.2775 | 0.0030 | 0.0071 |
| 414 | 0.0022 | 0.1341 | | | 0.4567 |
| 415 | 0.0023 | 0.0042 | 0.0424 | 0.0024 | 0.0126 |
| 416 | 0.0023 | 0.0862 | 1.238 | 0.0031 | 0.1043 |
| 417 | 0.0034 | 0.1187 | 0.0738 | 0.0060 | 0.0098 |
| 418 | 0.0027 | 0.0930 | 0.8615 | 0.0027 | 0.1596 |
| 419 | 0.0072 | 0.0072 | 0.0916 | 0.0083 | 0.0132 |
| 420 | 0.0029 | 3.126 | | | 6.028 |
| 421 | 0.0023 | 0.0127 | 0.1259 | 0.0009 | 0.0184 |
| 422 | 0.0023 | 0.1075 | | | 0.1384 |
| 423 | 0.0021 | 0.0042 | | | 0.0096 |
| 425 | | | | | 0.011 |
| 500 | 0.02194 | 0.367 | | | 2.497 |
| 501 | 0.02403 | 4.347 | | | 8.37 |
| 502 | 0.01213 | 0.575 | | | 0.0575 |
| 503 | 0.06757 | 1.763 | | | 7.515 |
| 504 | 0.01671 | 0.245 | | | 1.524 |
| 506 | 0.01708 | 0.397 | | | 0.471 |
| 507 | | 0.0896 | | | 1.883 |
| 508 | 0.04094 | 0.608 | | | 7.09 |
| 509 | | 6.125 | | | 6.821 |
| 510 | 0.007929 | 0.0979 | | | 0.0488 |
| 511 | 0.00238 | 0.401 | | | 1.831 |
| 512 | 0.002823 | | | | 0.150 |
| 513 | 0.003926 | 0.0385 | 0.986 | 0.004253 | 0.409 |
| 514 | 0.04745 | 0.272 | | | 48.48 |
| 515 | 0.01033 | 6.538 | | | 18.67 |
| 516 | 0.001907 | 0.0114 | | | 5.202 |
| 517 | 0.00265 | 0.0102 | 0.978 | 0.003379 | 0.175 |
| 518 | 0.00215 | 0.515 | | | 4.557 |
| 519 | 0.003719 | | | | 0.114 |
| 520 | 0.009473 | 0.0828 | 1.906 | 0.009237 | 0.593 |
| 521 | 0.006522 | 0.695 | | | 3.488 |
| 522 | 0.0239 | 0.0776 | | | 0.417 |
| 523 | 0.003199 | 11.7 | | | 12.42 |
| 524 | 0.007659 | 14.51 | | | 4.639 |
| 525 | | 15.11 | | | 3.136 |
| 526 | 0.007762 | 4.788 | | | 33.33 |
| 527 | 0.01546 | 3.061 | | | 33.33 |
| 528 | 0.008832 | | | | 8.806 |
| 530 | 0.01312 | 0.0499 | | | 0.488 |
| 531 | 0.009089 | 0.912 | 3.984 | 1.304 | 4.318 |
| 533 | 0.003732 | 0.0913 | | | 0.429 |
| 534 | 0.00692 | | | | 0.0200 |
| 535 | 0.008946 | 0.0295 | | | 0.0736 |
| 536 | 0.00272 | | | | 0.0219 |
| 537 | 0.003981 | 0.114 | | | 1.105 |
| 538 | 0.004704 | 0.00703 | 0.136 | 0.00326 | 0.0513 |
| 540 | 0.008588 | 2.138 | | | 0.147 |
| 541 | 0.01477 | 2.189 | 0.3026 | 0.02366 | 0.09599 |
| 542 | 0.003422 | 0.008551 | 0.1622 | 0.003713 | 0.03352 |
| 543 | 0.005435 | 0.0394 | 0.3108 | 0.008457 | 0.01109 |
| 544 | 0.008332 | 0.2905 | | | 0.03194 |
| 545 | 0.003552 | 0.02403 | 0.1099 | 0.005976 | 0.006445 |
| 546 | 0.01379 | 0.2684 | | | 0.05422 |
| 547 | 0.007 | 0.13 | 0.96 | 0.007 | 0.012 |
| 548 | 0.00758 | 0.06581 | 1.396 | 0.009684 | 0.02311 |
| 549 | 0.007154 | 0.5632 | | | 0.1691 |
| 550 | 0.03004 | 0.1538 | | | 0.04348 |
| 551 | 0.006293 | 0.01669 | 0.1717 | 0.007909 | 0.08474 |
| 552 | 0.009777 | 0.8388 | | | 0.4089 |
| 553 | 0.04414 | 0.0396 | 0.7083 | 0.04817 | 0.2183 |
| 554 | 0.0268 | 0.237 | 1.667 | 0.02263 | 0.2028 |
| 555 | 0.002391 | 0.1048 | | | 0.1966 |
| 556 | 0.05118 | 0.2506 | | | 0.9757 |
| 557 | 0.003105 | 0.004635 | 0.041 | 0.002491 | 0.01508 |
| 558 | 0.05348 | 0.3621 | | 0.03576 | 1.224 |
| 560 | 0.007101 | 0.1303 | 1.565 | 0.00993 | 0.05164 |
| 561 | | | | | 0.23 |
| 562 | | | | | 0.754 |
| 563 | 0.008354 | 0.03679 | 0.8452 | 0.006952 | 0.2559 |
| 564 | 0.003494 | 1.279 | | | |
| 565 | | | | | 0.17 |
| 566 | | | | | 0.31 |
| 567 | | | | | 0.011 |
| 570 | 0.008907 | 0.007186 | 0.4132 | 0.01023 | 0.02514 |
| 571 | | | | | 0.087 |
| 572 | | | | | 0.031 |
| 600 | 0.0256 | 0.5701 | | 0.1863 | 1.089 |
| 601 | 0.0008 | 3.999 | | | 1.242 |
| 602 | 0.0010 | 0.066 | 0.5970 | 0.0017 | 0.1429 |
| 603 | 0.0032 | 0.1833 | | | 0.4963 |
| 604 | 0.0013 | 0.0318 | 0.7042 | 0.0011 | 0.0624 |
| 605 | 0.0017 | 1.2590 | | | |
| 606 | 0.0026 | 0.1606 | | 0.0032 | 0.2861 |
| 607 | 0.0023 | 0.0096 | | 0.0031 | 0.8859 |
| 608 | 0.0035 | 0.0529 | | | 0.0310 |
| 609 | 0.0074 | 18.0400 | | | 0.9953 |
| 610 | 0.0124 | 0.4528 | | | 0.2722 |
| 611 | 0.0019 | 0.0115 | 0.4434 | 0.0014 | 0.1826 |
| 612 | 0.0016 | 0.4689 | | | 2.2110 |
| 613 | 0.0025 | 0.0157 | 0.9918 | 0.0023 | 0.3419 |
| 614 | 0.0015 | 2.2110 | | | |
| 615 | 0.0023 | 0.0450 | 0.8435 | 0.0019 | 1.0840 |
| 616 | 0.0029 | 0.0205 | 0.5378 | 0.002 | 0.2658 |
| 617 | 0.0011 | 1.3103 | | | 2.0410 |
| 618 | 0.0012 | 0.0140 | 0.1564 | 0.0014 | 0.0451 |
| 619 | 0.0028 | 0.0025 | 0.4830 | 0.0025 | 0.1020 |
| 620 | 0.0023 | 0.0059 | 0.1140 | 0.0019 | 0.0102 |
| 621 | 0.0031 | 0.1076 | | | 0.8345 |
| 622 | 0.0024 | 0.0044 | 0.0594 | 0.0022 | 0.0068 |
| 623 | 0.0024 | 0.0213 | | | 0.3447 |
| 624 | 0.0021 | 0.4937 | | | 0.4183 |
| 625 | 0.0019 | 0.0049 | 0.3589 | 0.0031 | 0.1455 |
| 626 | 0.00209 | 0.01299 | | | 0.1794 |
| 627 | 0.00302 | 0.0157 | | | 0.6309 |
| 628 | 0.003141 | 0.3571 | | | 0.9225 |
| 629 | 0.0011 | 0.0260 | 0.1043 | 0.0013 | 0.0157 |
| 630 | 0.0020 | 0.1751 | | | 0.1844 |
| 631 | 0.0022 | 0.0067 | 0.09082 | 0.0011 | 0.0081 |
| 633 | 0.0013 | 0.3064 | | | 0.3106 |
| 634 | 0.0023 | 0.0149 | 0.2375 | 0.0015 | 0.02698 |
| 635 | 0.0024 | 0.0272 | 0.7442 | 0.0024 | 0.0225 |
| 637 | 0.001526 | | | | 0.01762 |
| 638 | 0.001675 | | | | 0.1793 |
| 639 | 0.0051 | 0.0444 | 0.0444 | 0.0028 | 0.0478 |
| 640 | 0.0019 | 0.0106 | 0.2708 | 0.0011 | 0.0253 |
| 641 | 0.0033 | 0.0079 | 0.3060 | 0.0011 | 0.0244 |
| 642 | 0.0019 | 0.066 | | | 0.1986 |
| 643 | 0.0012 | 0.1793 | | | 0.3233 |
| 644 | 0.002572 | 0.0050 | 0.0889 | 0.0013 | 0.02718 |
| 645 | 0.0026 | 0.0222 | 0.2499 | 0.0024 | 0.0686 |
| 646 | 0.0002 | | | | 0.173 |
| 647 | 0.0026 | | | | 0.153 |
| 648 | 0.0036 | 2.0390 | | | 1.699 |
| 649 | 0.002 | 0.2398 | 0.4105 | 0.0012 | 0.2529 |
| 650 | 0.002 | 0.0057 | 0.0458 | 0.0014 | 0.0050 |
| 651 | 0.0013 | 0.0023 | 0.0178 | 0.0018 | 0.0081 |
| 652 | 0.0012 | 0.0331 | 0.3270 | 0.0017 | 0.0532 |
| 653 | 0.0009 | 0.001348 | 0.009106 | 0.001553 | 0.001437 |
| 654 | 0.0011 | 0.0095 | 0.0839 | 0.0020 | 0.0061 |
| 655 | 0.001595 | 0.2775 | | | 0.2402 |
| 656 | 0.0015 | 0.0025 | 0.0481 | 0.0028 | 0.0026 |
| 657 | 0.0025 | 0.1050 | 1.1440 | 0.0037 | 0.0941 |
| 658 | 0.0032 | 0.0089 | 0.0643 | 0.0084 | 0.0098 |
| 659 | 0.0035 | 0.01461 | 0.5347 | 0.001911 | 0.1222 |
| 660 | 0.00338 | 1.04 | | | |
| 700 | 0.015 | 1.17 | | | 0.39 |
| 701 | 0.003 | 0.085 | | | 0.15 |
| 702 | 0.0074 | 3.85 | | | 3.98 |
| 703 | 0.03 | 0.26 | | | 0.12 |
| 704 | 0.0029 | 0.45 | | 0.0029 | 1.07 |
| 705 | 0.0029 | 0.032 | 1.00 | 0.0024 | 0.033 |
| 706 | 0.023 | 2.45 | | | 4.61 |
| 707 | 0.0064 | 0.034 | | | 0.84 |
| 708 | 0.018 | 0.042 | | | 3.05 |
| 709 | 0.026 | 0.30 | | | 0.16 |
| 710 | 0.05 | 0.20 | | | 1.08 |
| 711 | 0.027 | 0.12 | | | 0.029 |
| 716 | 0.0038 | 1.28 | 28 | 0.004 | 2.325 |
| 717 | 0.024 | 0.2 | | | 1.16 |
| 718 | 0.0024 | 0.11 | | | 0.82 |
| 719 | 0.010 | 0.032 | | | 0.036 |
| 720 | 0.0019 | 0.52 | | | 3.99 |
| 721 | 0.0015 | 0.0045 | 0.17 | 0.0015 | 0.05 |
| 722 | 0.0064 | 0.009 | | 0.009 | 3.35 |
| 723 | 0.0027 | 0.046 | | | 0.080 |
| 724 | 0.063 | 0.37 | | | 15.3 |
| 725 | 0.035 | 0.058 | | | 0.76 |
| 726 | 0.0063 | 0.047 | | | 0.16 |
| 727 | 0.024 | 0.056 | | | 0.027 |
| 728 | 0.022 | 0.02 | | | 0.022 |
| 729 | 0.028 | 0.22 | | | 0.034 |
| 730 | 0.067 | 0.060 | | | 0.13 |
| 731 | 0.024 | 0.026 | | | 0.021 |
| 732 | 0.16 | 0.40 | | | 12.53 |
| 733 | 0.045 | 0.35 | | | 0.063 |
| 734 | 0.42 | 0.24 | | | 116 |
| 751 | 0.0033 | 0.51 | 0.996 | 0.029 | 0.027 |
| 752 | 0.0028 | 2.7 | | | 0.60 |
| 753 | 0.0057 | 0.06 | 0.639 | 0.002 | 0.023 |
| 754 | 0.0024 | 0.042 | 0.433 | 0.0026 | 0.039 |
| 755 | 0.0028 | 0.022 | 0.341 | 0.0019 | 0.023 |
| 756 | 0.0036 | 0.64 | 10.160 | 0.0022 | 0.60 |
| 757 | 0.0027 | 0.013 | 2.851 | 0.0030 | 2.4 |
| 758 | 0.005 | 0.17 | | | 0.43 |
| 762 | 0.0020 | 0.005 | 0.025 | 0.003 | 0.0048 |
| 763 | 0.0027 | 0.12 | 2.148 | 0.0025 | 0.062 |
| 764 | 0.0036 | 0.0044 | 0.024 | 0.0022 | 0.0052 |
| 800 | 0.018 | 3.0 | | | 2.3 |
| 801 | 0.007 | 0.099 | | | 1.0 |
| 805 | 0.010 | 0.010 | | 0.005 | 21.5 |
| 806 | 0.041 | 0.023 | | 0.009 | 42.9 |
| 807 | 0.003 | 0.025 | | | 2.0 |
| 808 | 0.026 | 1.2 | | | 0.32 |
| 809 | 0.016 | 0.82 | | | 4.2 |
| 810 | 0.004 | 0.018 | | | 0.005 |
| 811 | 0.042 | 0.058 | | | 0.664 |
| 812 | 0.034 | 0.44 | | | 0.15 |
| 813 | 0.043 | 0.038 | 1.4 | 0.087 | 0.018 |
| 814 | 0.10 | 0.055 | | | |
| 834 | 0.0842 | | | 0.0488 | 16.670 |
| 835 | 0.0035 | 0.943 | | 0.0014 | 1.313 |
| 836 | 0.0021 | 0.016 | 0.297 | 0.0018 | 0.060 |
| 837 | 0.0025 | 1.516 | | 0.0019 | |
| 838 | 0.0055 | | | 0.0013 | |
| 839 | 0.0009 | 0.865 | | 0.0023 | 0.971 |
| 841 | 0.0026 | 0.849 | | 0.0017 | 0.523 |
| 842 | 0.0066 | | | 0.0014 | |
| 843 | 0.0022 | 0.329 | | | |
| 844 | 0.0022 | 0.023 | 0.239 | 0.0026 | 0.213 |
| 846 | 0.0029 | 0.034 | 0.302 | 0.0028 | 0.039 |
| 847 | 0.0038 | 0.214 | 1.758 | 0.0050 | 0.312 |
| 848 | 0.0029 | 0.010 | 0.085 | 0.0045 | 0.026 |
| 849 | 0.0023 | 0.005 | 0.029 | 0.0029 | 0.005 |
| 850 | 0.0028 | 0.855 | | | 0.375 |
| 852 | >0.0030 | 1.319 | | 0.0025 | 0.058 |
| 853 | >0.0036 | 0.085 | 0.705 | 0.0042 | 0.011 |

Blank spaces indicate that no data was available.

1. A compound having the formula (I): 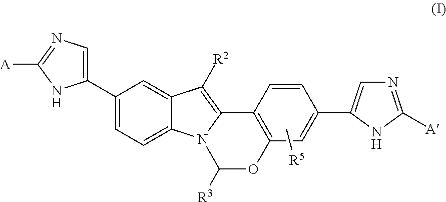
or a pharmaceutically acceptable salt thereof, wherein:
A is: 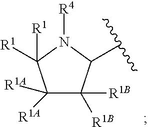
A′ is: 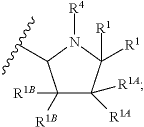
each occurrence of R¹ is independently selected from H, C₁-C₆ alkyl, C₃-C₇ cycloalkyl, C₁-C₆ haloalkyl and halo, or two R¹ groups that are attached to the same carbon atom, and the common carbon atom to which they are attached, can combine to form a spirocyclic C₃-C₇ cycloalkyl group;
each occurrence of R^{1A} is independently selected from H, C₁-C₆ alkyl, C₃-C₇ cycloalkyl, C₁-C₆ haloalkyl and halo, or one R^{1A} group and an R¹ group that are attached to same ring, together with the ring carbon atoms to which they are attached, can combine to form a fused C₃-C₇ cycloalkyl group, or two R^{1A} groups that are attached to the same carbon atom, and the common carbon atom to which they are attached, can combine to form a spirocyclic C₃-C₇ cycloalkyl group;
each occurrence of R^{1B} is independently H, C₁-C₆ alkyl, C₃-C₇ cycloalkyl, C₁-C₆ haloalkyl or halo, or an R^{1B} group and an R^{1A} group that are attached to the same ring, together with the carbon atoms to which they are attached, can combine to form a fused C₃-C₇ cycloalkyl group, or an R^{1B} group and an R¹ group that are attached to the same ring, can combine to form a bridging group having the formula —CH₂— or —CH₂CH₂—, or or two R^{1B} groups that are attached to the same carbon atom, and the common carbon atom to which they are attached, can combine to form a spirocyclic C₃-C₇ cycloalkyl group
R² is H, C₁-C₆ alkyl, C₃-C₇ cycloalkyl, phenyl or halo;
R³ is thiophenyl, wherein said thiophenyl group can be optionally substituted on one or more ring carbon atoms with R⁶;
each occurrence of R⁴ is independently selected from —C(O)O—(C₁-C₆ alkyl), —C(O)—C(R⁷)₂NHC(O)O—R⁸, —C(O)—CH(R⁷)(R⁸) and —C(O)—CH(R⁷)N(R⁹)₂;
R⁵ represents up to 2 substituents, each independently selected from H, halo, —CN, C₁-C₆ alkyl, C₁-C₆ haloalkyl, —(C₁-C₆ alkylene)ₘ-C₃-C₇ cycloalkyl, 4 to 6-membered monocyclic heterocycloalkyl, 5 or 6-membered monocyclic heteroaryl, C₆-C₁₀ aryl, benzyl and —O—(C₁-C₆ alkyl), wherein said C₃-C₇ cycloalkyl group, said 4 to 6-membered monocyclic heterocycloalkyl group, said 5 or 6-membered monocyclic heteroaryl group, said C₆-C₁₀ aryl group, or the phenyl moiety of said benzyl group can be optionally substituted with up to 3 groups, which can be the same or different, and are selected from halo, —CN, C₁-C₆ alkyl, C₁-C₆ haloalkyl, —O—C₁-C₆ alkyl, —(C₁-C₆ alkylene)-O—C₁-C₆ alkyl and —O—(C₁-C₆ haloalkyl);
R⁶ represents up to 2 substituents, each independently selected from halo, —CN, C₁-C₆ alkyl, —C(O)OH, C₁-C₆ haloalkyl, —O—(C₁-C₆ haloalkyl), C₂-C₆ alkynyl, C₁-C₆ hydroxyalkyl, —O—C₁-C₆ alkyl, —(C₁-C₆ alkylene)-O—(C₁-C₆ alkyl), —N(R⁶)₂, —C(O)N(R⁶)₂, optionally substituted C₆-C₁₀ aryl, —(C₁-C₆ alkylene), —(C₃-C₇ cycloalkyl), —O—(C₆-C₁₀ aryl), —(C₂-C₆ alkynyl)-(C₃-C₇ cycloalkyl), 4 to 7-membered monocyclic heterocycloalkyl, 5 or 6-membered monocyclic heteroaryl, —O-(5 or 6-membered monocyclic heteroaryl), 8 to 10-membered bicyclic heteroaryl and —O-(8 to 10-membered bicyclic heteroaryl), wherein said C₆-C₁₀ aryl group, said C₃-C₇ cycloalkyl group, said 4 to 7-membered monocyclic heterocycloalkyl group, said 5 or 6-membered monocyclic heteroaryl group and said 8 to 10-membered bicyclic heteroaryl group, can be optionally substituted with up to 3 groups, each independently selected from halo, hydroxy, C₁-C₆ alkyl, C₁-C₆ haloalkyl and —O—C₁-C₆ alkyl, and wherein said C₆-C₁₀ aryl group, said 5 or 6-membered monocyclic heteroaryl group and said 9- or 10-membered bicyclic heteroaryl group, can be optionally fused with a 3 to 6 membered cycloalkyl group; and wherein said thiophenyl group can be optionally fused to a benzene ring, a 5 or 6-membered monocyclic heterocycloalkyl group, a 5 or 6-membered monocyclic heteroaryl group or a C₅-C₆ cycloalkyl group, wherein said 5 or 6-membered monocyclic heterocycloalkyl group, said 5 or 6-membered monocyclic heteroaryl group and said C₅-C₆ cycloalkyl group can form a spirocycle with a C₃-C₇ cycloalkyl group or a 4 to 7-membered monocyclic heterocycloalkyl group each occurrence of R⁷ is independently selected from H, C₁-C₆ alkyl, C₁-C₆haloalkyl, —(C₁-C₆ alkylene)-O—C₁-C₆ alkyl, phenyl, 4 to 8-membered monocyclic heterocycloalkyl, 6 to 10-membered bicyclic heterocycloalkyl and —(C₁-C₆ alkylene)ₘ-C₃-C₇ cycloalkyl, wherein said 4 to 8-membered monocyclic heterocycloalkyl group, said 6 to 10-membered bicyclic heterocycloalkyl group and said C₃-C₇ cycloalkyl group can be optionally substituted with up to 5 groups, each independently selected from halo, —CN, C₁-C₆ alkyl, C₃-C₇ cycloalkyl, C₁-C₆ haloalkyl, —O—C₁-C₆ alkyl, —N(R⁶)₂ and —O—(C₁-C₆ haloalkyl), and wherein said C₃-C₇ cycloalkyl group can be optionally fused to a 4 to 6-membered monocyclic heterocycloalkyl group, and wherein said 4 to 8-membered monocyclic heterocycloalkyl group and said C₃-C₇ cycloalkyl group can be substituted on a ring carbon atom with a spirocyclic C₃-C₆ cycloalkyl group; and wherein said C₃-C₇ cycloalkyl group can be substituted on a ring carbon atom with a spirocyclic 3 to 6-membered monocyclic heterocycloalkyl group, and wherein two R⁷ groups, that are attached to a common carbon atom, together with the common carbon atom to which they are attached, join to form a C₃-C₇ cycloalkyl group;
each occurrence of R⁸ is independently selected from C₁-C₆ alkyl, C₃-C₇ cycloalkyl and C₆-C₁₀ aryl;
each occurrence of R⁹ is independently selected from H, C₁-C₆ alkyl, C₃-C₇ cycloalkyl and C₆-C₁₀ aryl; and
each occurrence of m is independently 0 or 1.

2. The compound of claim 1, wherein R³ is: 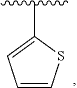 [SMILES: *c1cccs1]
which can be optionally substituted as described in claim 1.

3. The compound of claim 1, wherein each occurrence of R⁴ is independently —C(O)—C(R⁷)₂NHC(O)O—R⁸.

4. The compound of claim 1, wherein A and A′ are each independently selected from:

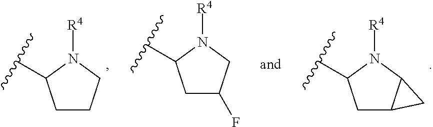

5. The compound of claim 4, wherein each occurrence of R⁴ is independently —C(O)CH(R⁷)—NHC(O)O—(C₁-C₆ alkyl) and R⁷ is C₁-C₆ alkyl, C₁-C₆ haloalkyl or 4 to 6-membered monocyclic heterocycloalkyl, wherein said 4 to 6-membered monocyclic heterocycloalkyl group can be optionally substituted with up to five C₁-C₆ alkyl groups or said 4 to 6-membered monocyclic heterocycloalkyl group can be optionally substituted on a ring carbon atom with a spirocyclic C₃-C₆ cycloalkyl group.

6. The compound of claim 1 having the formula: 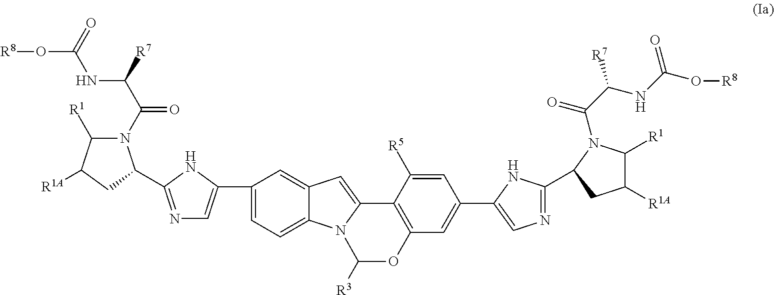
or a pharmaceutically acceptable salt thereof,
wherein:
each R¹ is H;
each R^{1A} is H, or an R^{1A} groups and an R¹ group that are attached to same ring, together with the ring carbon atoms to which they are attached, can combine to form a fused cyclopropyl group;
R³ is: 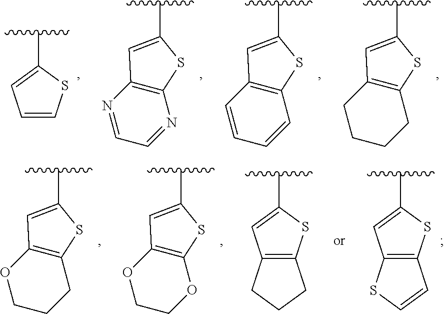
wherein R³ can be optionally substituted on one or more ring carbon atoms with a group selected from methyl, ethyl, n-propyl, isopropyl, t-butyl, F, —CHF₂, —CH₂CF₃, —CH₂F, —CF₃, cyclopropyl, cyclobutyl, cyclopentyl, —CH₂-cyclopropyl, methoxy, —O-(halo-substituted phenyl), —OCF₃, —C(CH₃)₂OH, —CH₂CH₂OCH₃, halo-substituted phenyl and —CN;
each occurrence of R⁵ is independently selected from H, methyl and F;
each occurrence of R⁷ is independently selected from C₁-C₆ alkyl, C₁-C₆ haloalkyl and 4 to 6-membered monocyclic heterocycloalkyl, wherein said 4 to 6-membered monocyclic heterocycloalkyl group can be optionally substituted with up to 5 groups, each independently selected from halo, C₁-C₆ alkyl and C₃-C₂ cycloalkyl, and wherein said 4 to 6-membered monocyclic heterocycloalkyl group can be optionally substituted on a ring carbon atom with a spirocyclic C₃-C₆ cycloalkyl group;
each occurrence of R⁸ is independently C₁-C₆ alkyl.

7. The compound of claim 6, having the formula: 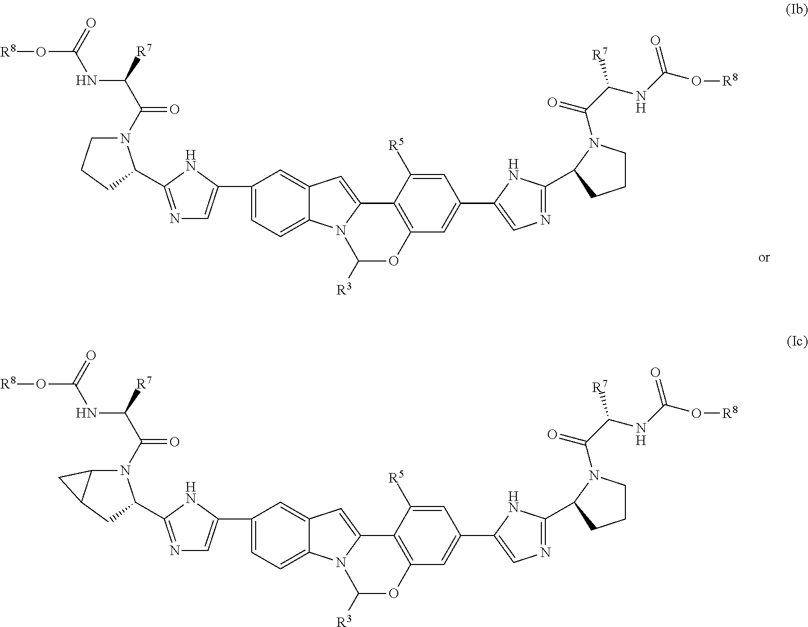
or a pharmaceutically acceptable salt thereof,
wherein:
R³ is: 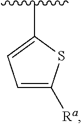
R^{a} is C₁-C₆ alkyl or C₃-C₇ cycloalkyl;
R⁵ is H or F;
each occurrence of R⁷ is independently selected from C₁-C₆ alkyl, C₁-C₆ haloalkyl and tetrahydropyranyl, wherein said tetrahydropyranyl group can be optionally substituted with up to 5 groups, each independently selected from halo, C₃-C₇ cycloalkyl or C₁-C₆ alkyl, and wherein said tetrahydropyranyl group can be optionally substituted on a ring carbon atom with a spirocyclic cyclopropyl group.
each occurrence of R⁸ is methyl.

8. The compound of claim 1, wherein each occurrence of R⁷ is independently selected from isopropyl, —CF(CH₃)₂, 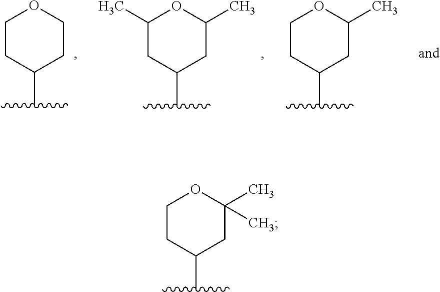
and each occurrence of R⁸ is methyl.

9. The compound of claim 7, wherein R^{a} is cyclopropyl, ethyl, cyclopentyl, n-propyl, isopropyl, t-butyl or isobutyl; and R⁵ is F.

10. Any compound numbered 1-853 as described in the above specification or stereoisomer thereof, or a pharmaceutically acceptable salt thereof.

11. A pharmaceutical composition comprising an effective amount of the compound of claim 1 and a pharmaceutically acceptable carrier.

12. The pharmaceutical composition according to claim 11 further comprising a second therapeutic agent selected from the group consisting of HCV antiviral agents, immunomodulators, and anti-infective agents.

13. The pharmaceutical composition according to claim 12, further comprising a third therapeutic agent selected from the group consisting of HCV protease inhibitors, HCV NS5A inhibitors and HCV NS5B polymerase inhibitors.

14. (canceled)

15. A method of treating a patient infected with HCV comprising the step of administering an amount of (i) the compound according to claim 1, effective to treat infection by HCV in said patient.

16. The method according to claim 15, further comprising the step of administering pegylated-interferon alpha and an HCV protease to said patient.

17. The method according to claim 15, further comprising the step of administering ribavirin to said patient.

18. The method according to claim 15, further comprising the step of administering from one to three additional therapeutic agents to said patient, wherein the additional therapeutic agents are each independently selected from HCV protease inhibitors, HCV NS5A inhibitors and HCV NS5B polymerase inhibitors.

19. The method according to claim 18, wherein the one to three additional therapeutic agents comprises MK-5172.

20. The method according to claim 18, wherein the one to three additional therapeutic agents comprises sofosbuvir.
